(19) United States
(12) Patent Application Publication
(10) Pub. No.: US 2025/0339409 A1
(43) Pub. Date: Nov. 6, 2025

(54) USES OF PAN BET INHIBITORS

(71) Applicant: VYNE THERAPEUTICS INC., Bridgewater, NJ (US)

(72) Inventors: Iain STUART, Flanders, NJ (US); Russell ELLIOTT, Basking Ridge, NJ (US); Yohan HAZOT, Kiryat Ekron (IL); Ariel MARGULIS, Jerusalem (IL); Christopher Andrew WOODLAND, Dundee (GB); Mark BELL, Dundee (GB)

(21) Appl. No.: 18/705,240

(22) PCT Filed: Nov. 2, 2022
(86) PCT No.: PCT/US2022/079171

§ 371 Date: Apr. 26, 2024

Related U.S. Application Data
(60) Provisional application No. 63/263,511, filed on Nov. 3, 2021.
(60) Provisional application No. 63/268,839, filed on Mar. 3, 2022.
(60) Provisional application No. 63/362,122, filed on Mar. 29, 2022.
(60) Provisional application No. 63/362,780, filed on Apr. 11, 2022.
(60) Provisional application No. 63/364,770, filed on May 16, 2022.
(60) Provisional application No. 63/369,455, filed on Jul. 26, 2022.

(51) Int. Cl. A61K 31/437; A61K 47/34; A61K 47/36; A61K 47/38; A61P 17/02; A61P 19/02; A61P 29/00; C07D 471/04

(52) U.S. Cl. CPC A61K 31/437; A61K 47/34; A61K 47/36; A61K 47/38; A61P 17/02; A61P 19/02; A61P 29/00; C07D 471/04

(57) ABSTRACT

The present disclosure relates to methods for the treatment, amelioration, or prophylaxis of an inflammatory and/or an autoimmune disease or disorder or a disease or disorder related thereto (e.g., wounds, pigmentation or pigmentation related diseases and disorders, joint or joint related diseases and disorders, respiratory or respiratory related diseases or disorders; and fibrosis or fibrosis associated diseases and disorders) using soft BET inhibitors.

### CROSS-REFERENCE TO RELATED APPLICATIONS

This application is a national stage entry of PCT/US2022/079171, which claims priority to U.S. Provisional Application No. 63/263,511, filed on Nov. 3, 2021, now expired; U.S. Provisional Application No. 63/268,839, filed on Mar. 3, 2022, now expired; U.S. Provisional Application No. 63/362,122, filed on Mar. 29, 2022, now expired; U.S. Provisional Application No. 63/362,780, filed on Apr. 11, 2022, now expired; U.S. Provisional Application No. 63/364,770, filed on May 16, 2022, now expired; and U.S. Provisional Application No. 63/369,455, filed on Jul. 26, 2022, now expired; the contents of each of which are hereby incorporated by reference in their entirety.

### FIELD OF THE DISCLOSURE

The present disclosure is directed to methods for the treatment or prevention of inflammation and inflammation related diseases or disorders, autoimmune and autoimmune related diseases or disorders, wounds and wound related diseases or disorders, pigmentation or pigmentation related diseases or disorders, joint related diseases and disorders, respiratory or respiratory related diseases or disorders, fibrosis or fibrosis-associated diseases or disorders using BET inhibitors and formulations, such as topical and intra-articular formulations, of diseases and disorders. The BET inhibitors are PAN BET inhibitors that can bind to BDI and BDII. The BET inhibitors are soft BET inhibitors.

### BACKGROUND

Diseases and disorders may be multifactorial. They can involve inflammation or can result in inflammation related disorders. Autoimmune diseases and disorders may result in inflammation or may result in inflammation related disorders. An inflammatory or autoimmune disease or disorder may cause or result in changes, damage and/or wounds. A significant aspect of treatment of many diseases or disorders is to facilitate correct healing. A failed or failing healing process may, for example, leave lesions, wounds and/or fibrosis, Pyoderma gangrenosum (PG) is a life-threatening, severe autoimmune neutrophilic dermatosis causing significant dermal ulceration with a prevalence in the U.S. of 5.8 per 100,000². The condition predominantly affects adults, but childhood cases are rarely reported. The sex incidence ranges from being equal, to females being predominantly affected in up to 76% of cases. Classical PG presents most commonly as an extremely painful erythematous lesion which rapidly progresses to a blistered or necrotic ulcer. There is often a ragged undermined edge with a violaceous/erythematous border. The lower legs are most frequently affected although PG can present at any site. The lesion may be precipitated by minor trauma. Most cases of PG are of the classic ulcerative type (approximately 85%), but other subtypes include bullous, vegetative, pustular, peristomal and superficial granulomatous variants, with subtypes of PG sometimes transitioning from one form to another. The differential diagnosis includes all other causes of cutaneous ulceration as there are no definitive laboratory or histopathological criteria for PG. The pathogenesis of PG remains unclear however it is recognized that neutrophils play a key role in the disease process. Upregulation of several key proinflammatory and neutrophil chemotactic factors within lesions have been identified and these include IL-1β, IL-17, TNFα, IL-8, IL-6, IL-18, INFγ, IL-36γ, and IL-23. IL-8 has been demonstrated to produce PG in animal models. IL-8 is also induced in fibroblasts of PG ulcers and its associated ligands are over-expressed in PG.

An important aspect of treatment of PG is wound healing. In many cases, the healing of a wound is imperfect, resulting in the formation of a scar. Attempts to accelerate the healing process may result in elevating the incidence of scar formation. When the wound is bacterially infected, the healing process becomes more challenging and may take longer. Scars are more often caused following improper treatment.

Despite being a well-recognized condition there is often a failure to make an early diagnosis of PG. Potent topical corticosteroids and tacrolimus ointment applied to the ulcer surface are useful and intralesional injections of corticosteroid into the erythematous active border may be considered. In more severe disease, systemic therapy is required. Oral corticosteroids are the mainstay of treatment and are used to gain rapid control. Ciclosporin is used either alone or in combination with corticosteroids as a steroid-sparing agent, in cases where prolonged treatment is required. Other systemic treatments utilized with varying success include colchicine, sulphasalazine, dapsone, minocycline, apremilast and thalidomide.

Some limitations of current therapies include inadequate efficacy of nonsteroidal topical treatments, restrictions on application to particular body regions, “steroid and CNI phobia,” and application site reactions. Potential long-term safety concerns include systemic side-effects and skin atrophy (for striae and other atrophic changes) with topical corticosteroids and increased risk of infections with CNIs.

Generalized Pustular Psoriasis (GPP) is a rare, debilitating, and often life-threatening inflammatory disease characterized by episodic infiltration of neutrophils into the skin, pustule development, and systemic inflammation, which can manifest in the presence or absence of chronic plaque psoriasis. IL-1β, IL-17, IL-8 and IL-36γ are the dominant cytokines increased in GPP. Current treatments are unsatisfactory and warrant a better understanding of GPP pathogenesis.

Palmar Plantar Pustulosis (PPP) is is a chronic dermatitis characterized by intra-epidermal vesicles pustules containing neutrophils (PMN), located on the palms and soles. Although several pathogeneses of PPP such as concomitant tonsillitis, periodontitis or metal allergy have been proposed, the aetiology of PPP remains unknown. IL-1, IL-8, IL-17 and IL-36γ are the dominant cytokines increased in PPP. Current treatments are unsatisfactory and warrant a better understanding of PPP pathogenesis.

Many other topical disorders involve inflammation and share similar biomarker patterns and a product which is capable of reducing inflammatory cytokines involved in inflammation and treats or ameliorates the disorder while avoiding or minimizing systemic and skin-related side effects would be advantageous and could improve patient compliance with treatment.

Autoimmune skin diseases that are characterized by patches of varying pigmentation like vitiligo are challenging to treat. Vitiligo, for example, is a disease where the immune system attacks pigment cells in the body. It can be found anywhere in the body and can range from mild to severe. Vitiligo is an acquired, chronic depigmenting disorder of the skin characterized by patchy loss of skin color e.g., with the skin becoming white usually with sharp margins because of pigment loss. Sometimes the skin and the inside of the mouth and nose are involved. Typically, both sides of the body are affected. Often the patches begin on areas of skin that are exposed to the sun. It is more noticeable in people of color. Although not life-threatening, vitiligo may result in psychological stress and those affected are sometimes stigmatized.

The exact cause of vitiligo is unknown. Without being bound by theory, Vitiligo is typically considered a multifactorial disorder, ultimately leading to the loss of functional melanocytes via autoimmunity (see, e.g., Boniface K, et al. Clin Rev Allergy Immunol. 2018; 54:52-67; Seneschal J, et al. Pigment Cell Melanoma Res. 2021; 34:236-243; Chen J, et al. Med Res Rev. 2021; 41:1138-1166; Frisoli M, et al. Annu Rev Immunol. 2020; 38:621-648). Loss of melanocytes is the pathological hallmark of vitiligo, which may be induced by exaggerated immune response, including autoreactive CD8 T cells producing high levels of type 1 cytokines. However, the interplay between this inflammatory response and melanocyte disappearance remains to be fully characterized. It is hypothesized that melanocytes disappear in vitiligo because of melanocytorhagy, defective melanocyte adhesion to basal membrane. It is also postulated that genetic susceptibility and environmental factors both may play a role. Studies suggest that changes in the immune system are responsible for the condition. Variations in genes that are part of the immune system or part of melanocytes have both been associated with vitiligo. Inflammatory interleukin-1β and interleukin-18 are expressed at high levels in people with vitiligo. Vitiligo is also thought to be caused by the immune system attacking and destroying the melanocytes of the skin. Vitiligo is sometimes associated with autoimmune and inflammatory diseases such as Hashimoto's thyroiditis, scleroderma, rheumatoid arthritis, type 1 diabetes mellitus, psoriasis, Addison's disease, pernicious anemia, alopecia areata, systemic lupus erythematosus, and celiac disease.

Most cases are non-segmental, meaning they affect both sides; and in these cases, the affected area of the skin typically expands with time. About 10% of cases are segmental, meaning they mostly involve one side of the body; and in these cases, the affected area of the skin typically does not expand with time.

Vitiligo is the most common depigmenting skin condition, with a prevalence estimated at 1% of the world population and, in some populations, it affects as many as 2-3%. Males and females are equally affected. About half show the disorder before age 20 and most develop it before age 40.

There is no known cure for vitiligo. Current treatments of vitiligo are unsatisfactory. First-line vitiligo treatment includes topical preparations of immune suppressing medications such glucocorticoids (such as 0.05% clobetasol or 0.10% betamethasone) and calcineurin inhibitors (such as tacrolimus or pimecrolimus). For those with lighter skin, sunscreen and makeup are typically recommended. For those with darker skin, treatment options may include phototherapy to darken the light patches or hydroquinone to lighten the unaffected skin, however the latest is less recommended due to increased risk of skin cancer. If light therapy and medications have not worked, some people with stable disease may be candidates for surgery (skin grafting, blister grafting and cellular suspension transplant). Currently, there are no FDA-approved re-pigmenting treatments for vitiligo.

Joint or joint related disorders or diseases are diseases that affect human joints. Arthritis is one example of a well-known joint disease. Osteoarthritis is the most common form of arthritis and involves the wearing away of the cartilage that caps the bones in a person's joints. Rheumatoid arthritis (RA) is a disease in which the immune system attacks the joints, beginning with the lining of joints. RA is the most frequent autoimmune chronic inflammatory rheumatism, primarily affecting the synovial membrane of multiple joints. Although its etiology is still unknown, it is now acknowledged that during the inflammatory process of arthritis there are three key mediators, the proinflammatory cytokines TNF-α, IL-1β and IL-6 (see Mori T. et al., IL-1β and TNFα-initiated IL-6-STAT3 pathway is critical in mediating inflammatory cytokines and RANKL expression in inflammatory arthritis, International Immunology, Volume 23, Issue 11, 2011, pp. 701-712).

Regardless of the cause, inflammation of the joints may cause pain, stiffness, swelling, and some redness of the skin about the joint. Steroids (i.e., corticosteroids) are synthetic drugs that are used to treat a variety of inflammatory diseases and conditions. But the administration of corticosteroids, particularly for extended periods of time, can have a number of unwanted side effects or adverse reactions. The effectiveness of corticosteroids generally diminishes with time and there are disadvantages in their use, including a greater susceptibility to infection and peptic ulcers and corticosteroid injection directly into joint tissues may in some subjects worsen joint damage. For example, the unwanted adverse reactions of triamcinolone (which is a corticosteroid) injections include, hypersensitivity reactions, such as anaphylaxis, joint infection and damage, increased risk of infections, alterations in endocrine function, cardiovascular and renal effects, increased intraocular pressure, gastrointestinal perforation, alternations in bone density and behavioral and mood disturbances. As yet another example, the unwanted adverse reactions of dexamethasone (another corticosteroid) include, fluid and electrolyte disturbances, musculoskeletal, gastrointestinal, neurologic, dermatologic, endocrine, ophthalmic, metabolic cardiovascular, anaphylactoid or hypersensitivity reactions, thromboembolism, weight gain, increased appetite, and nausea (see, e.g., Brinks, A. et al. “Adverse effects of extra-articular corticosteroid injections: a systematic review.” BMC musculoskeletal disorders, 11:206, 2010).

Moreover, other autoimmune diseases and other diseases that are characterized by fibrosis or scarring, such as Pulmonary Fibrosis (PF), are challenging to treat. As an example, PF is a chronic disease which affects at least 5 million people globally showing aberrant remodeling of lung tissue. PF is part of a larger group of more than 200 interstitial lung diseases (also known as ILDs or diffuse parenchymal lung disease (DPLD)) that are characterized by inflammation and/or scarring in the lung. In ILDs, the injury/damage occurs in the walls of the air sacs (alveoli) of the lung, as well as in the tissue and space around these air sacs (interstitium). When an ILD includes scar tissue in the lung, it is known as PF.

Defined by the pathological accumulation of extracellular matrix (ECM) proteins, fibrosis results in scarring which may be coupled with thickening of the affected tissue it is in essence an exaggerated wound healing response which interferes with normal organ function. Fibrosis of the lung is generally characterized by alveolar epithelial cell injury, areas of type II cell hyperplasia, accumulation of fibroblasts and myofibroblasts, and the deposition of extracellular matrix proteins. The result is a progressive loss of normal lung architecture and impairment in gas exchange. Accordingly, symptoms can include shortness of breath, a dry cough, feeling tired, weight loss, and nail clubbing (e.g., due to low oxygen in the blood).

There are many different types of PF that fall into six primary categories. Five are based on the type of induction or exposure, viz: environmental, occupational, drug-induced, radiation-induced, and autoimmune lung disease. The other main category idiopathic PF (IPF) is where no cause can be identified.

Lung damage (scar tissue) caused by PF cannot be repaired and currently approved medications have limited efficacy and suffer from multiple side effects. Medications and therapies can sometimes improve quality of life, help ease symptoms, or slow down the worsening of scarring. Lung transplantation is the only therapeutic option available in severe cases. Supplemental oxygen, pulmonary rehabilitation, and management of symptoms are important treatment options for many types of pulmonary fibrosis, depending on severity.

In addition to fibrosis in lung diseases and disorders, fibrosis can be a major problem throughout the body including in organs, such as the kidney, liver, heart, lymph nodes (e.g., mediastinal fibrosis), bone marrow, skin, tendons, joints, connective tissue, soft tissues, and cavities e.g., retroperitoneal. Fibrosis may be local, or it can be systemic.

Many disorders, such as topical disorders, involve inflammation and share similar biomarker patterns and a product which is capable of reducing inflammatory cytokines involved in inflammation and treats or ameliorates the disorder while avoiding or minimizing side effects or adverse reactions, such as systemic or skin-related side effects, would be advantageous and could improve patient compliance with treatment.

Bromodomain and Extra-Terminal (BET) proteins are a family of four bromodomain-containing (BRD) proteins (BRD2, BRD3, BRD4 and BRDT), each family member containing two BRDs (located next to each other toward the N-terminal of the proteins) and an extra-terminal domain. The two BRDs are designated binding domain I (BDI) and binding domain II (BDII). The BET proteins are transcriptional regulators that bind via their hydrophobic pocket to acetylated lysines on the tails of histones H3 and H4 and regulate chromatin structure and function. BET proteins ‘read’ aceylated lysines, enable chromatin remodelling and recruit transcription factors. In other words, BRDs are responsible for transducing the signals carried by acetylated lysine residues into various phenotypes and play a key role in regulating gene transcription via epigenetic interactions (“reading”) between the bromodomains and acetylated histones during cell proliferation and differentiation. For example, BRD4 recruits the transcription factor P-TEFb to promoters leading to altered expression of genes involved in the cell cycle.

BET proteins are ubiquitously expressed in humans except for BRDT, which is normally expressed in the testes but is also expressed by some cancers. BET proteins are known to have roles in the regulation of biochemical pathways such as MYC, BCL2, FOSL1, P-TEFb, NFkB, Glucocorticoid signaling and others. As such, there is growing interest in BET inhibitors as therapeutic targets for a wide range of diseases such as inflammatory diseases, cancers, infections, metabolic diseases, CNS disorders, fibrotic diseases and cardiac diseases. By modulating gene expression, they can potentially treat diseases that are at least in part caused by abnormal regulation of BET protein activity.

Several small molecules have been reported to be effective in BET inhibition, including diazepine-, 3,5-dimethylisoxazole-, thiazol-2-one-, diazobenzene-, and 4-acylpyrrole-based compounds. Compounds comprising 6-methyl-1,6-dihydro-7H-pyrrolo[2,3-c]pyridin-7-one moieties substituted at the 4- and/or 2-positions have been described as useful for the inhibition of BET proteins as well. To date, primary research related to BET inhibitors has focused on oncology and cardiovascular disease.

There are autoimmune skin diseases that are characterized by chronic ulcers and blisters which are challenging to treat and can potentially lead to life-threatening infections and life-changing surgical intervention. In addition to effectively reducing intra-lesional inflammatory burden, it is important that a treatment does not negatively impact innate skin repair mechanisms which could delay lesion closure. Although glucocorticosteroids are frequently used to treat neutrophilic dermatoses, prolonged use with topical products is associated with tachyphyaxis, while systemic use is known to significantly delay the time to ulcer closure with the potential for significant hormone-related systemic adverse events (Guo S and Dipietro L A, J Dent Res 2010 89(3):219-229; Tan S Y, Chandran N S and Ci-En Choi E, Clin Drug Invest. 2021 41:835-842; Wang A S, Armstrong E J and Armstrong A W, Amer J Surg. 2013 203(3):410-417; Paragliola R M, Papi G, Pontrcorvi A and Corsello S M, Int. J Mol Sci 2017 18(10):2201). With these limitations, the long-term use of glucocorticosteroids could extend patients exposure to the risks and complications associated with these serious, chronic conditions.

There remains an unmet need for topically applied BET inhibitors which positively impact diseases involving e.g., multiple, diverse inflammatory cell signaling pathways, including inflammation and in rare and other diseases, where many patients suffer with no adequate treatment options.

For example, IPF is a progressive inflammatory disorder driven by a fibrotic cascade of events including epithelial to mesenchymal transition, extracellular matrix production and collagen formation in the lungs. First-line treatment drugs are FDA approved pirfenidone and nintedanib (tyrosine kinase inhibitors) but neither the quality of life nor survival rates have been improved because of patient noncompliance due to multiple side effects (see, e.g., Andugulapati S B et al. Biochanin-A ameliorates pulmonary fibrosis by suppressing the TGF-β mediated EMT, myofibroblasts differentiation and collagen deposition in in vitro and in vivo systems. Phytomedicine, 2020).

A product that requires a short treatment period, which is safe, well-tolerated, and prevents occurrence and/or reduces the grade of severity or the incidences, for example, of disorders like PG, PPP, GPP, and scarring, pigmentation disorders like vitiligo, joint or joint related disorders or diseases, or lung diseases and disorders, for example, of fibrosis conditions like PF and other lung diseases and disorders that involve scarring, while avoiding unwanted side effects and adverse reactions would be advantageous and could improve patient compliance with treatment. Accordingly, there is a medical need to replace corticosteroids with safer and better drugs in order to reduce the systemic side effects associated with the administration of corticosteroids. In addition, there is a medical need to slow, arrest, reverse, or otherwise inhibit structural damage to tissues caused by inflammatory diseases, such as damage to articular tissues resulting from, for example, osteoarthritis or rheumatoid arthritis. Involvement of bursas, tendons, and tendon sheaths can be part of arthritic disease.

The present disclosure provides a specific genus of BET inhibitors (hereinafter BETi) that have been found to be surprisingly effective against diseases involving multiple, diverse inflammatory cell signaling pathways, in inflammation and in rare and other diseases, as well as joint or joint related disorders or diseases. More particularly, the present disclosure provides exemplary BETi that can provide a new and effective treatment and relief for pigmentation (related) diseases or disorders (such a vitiligo), or, alternatively, joint disorders or diseases, such as arthritis, bursitis, Ehlers-Danlos syndrome, epicondylitis, Felty syndrome, gouty arthritis, psoriatic arthritis, osteoarthritis, rheumatoid arthritis, Still's disease, tenosynovitis, synovitis, Sjögren's Syndrome, lyme disease, Whipple disease, bone cancer, lupus or other autoimmune joint disorders.

The present disclosure provides BETi that can provide new and effective treatment and relief for joint related diseases and disorders. Joints may be infected by many types of microorganisms (bacteria, fungi, viruses) and occasionally by animal parasites. Infection related joint diseases and disorders include infection by direct contamination, by way of the bloodstream e.g., through the synovial blood vessels, and by extension from adjacent bony infections (osteomyelitis). Infectious arthritis may affect one joint (monarthritis) or a few joints (oligoarthritis) rather than many (polyarthritis). Joints or parts thereof can be damaged e.g., cartilage by for example through staphylococci, hemolytic streptococci, and pneumococci infections, e.g., bone through tuberculosis such as tuberculous spondylitis (Pott disease), or through coccidioides immitis, brucellosis, such as brucella suis, leprosy (Hansen disease), rubella (German measles) and serum hepatitis, viral synovitis, dranunculiasis (Guinea worm disease), sexually transmitted diseases, including gonorrhea, reactive arthritis (Reiter disease), congenital syphilis such as Clutton joint lesion, and Yaws, which leads to skeletal lesions. Inflammation may destroy the joint cartilage and underlying bone and cause irreparable deformities. Adhesions between the articulating members are frequent in such cases, and the resulting fusion with loss of mobility is called ankylosis such as ankylosing spondylitis, (Marie-Strumpell disease or Bechterew disease). Another type of arthritis is associated with chronic intestinal diseases—ulcerative colitis, regional enteritis, inflammatory bowel disease, cirrhosis, and Whipple disease.

In addition to joint disorders and diseases resulting from any of the above, the present disclosure provides BETi that may also provide new and effective treatment or relief for noninflammatory joint diseases, injury and degenerative disorders. Trauma to joints includes blunt injuries, mild sprains, fractures and dislocations, ligamentous, tendinous, and capsular tears, tears in the semilunar cartilages (menisci), and hemarthrosis. Degenerative joint disease includes osteoarthritis, arthrosis deformans, precocious osteoarthritis congenital dysplasia malum coxae senilis, spondylosis, chrondromalacia patellae, metabolic diseases such gouty arthritis, podagra, ochronotic arthropathy, chondrocalcinosis, or pseudogout, mucopolysaccharidoses, Hurler syndrome, Morquio disease, and polyepiphyseal dysplasias.

The present disclosure provides BETi that may also provide new and effective treatment or relief for secondary joint diseases and disorders, including hemorrhagic joints, hemarthrosis, villonodular synovitis, joint diseases that arise in association with aseptic necrosis e.g., can occur with fractures, osteochondritis dissecans, slipped epiphysis, Osgood-Schlatter, Legg-Calvé-Perthes, endocrine-malfunctioning resultant joint disorders, acromegaly, neurogenic arthropathy, Charcot joint, hypertrophic osteoarthropathy, reflex sympathetic dystrophy, joint tumors, synovial chondromatosis, cartilaginous nodules, synovial osteochondromatosism, synoviomas, synovial sarcomas, and polymyalgia rheumatica.

The present disclosure additionally provides exemplary BET inhibitors that can provide a new and effective treatment for a fibrotic or scarring (related) disease or disorder such as a lung or pulmonary fibrosis by targeting TGF-0 mediated cascade of fibrotic events and thereby improving the survival of PF patients.

### BRIEF SUMMARY

The present disclosure relates to the treatment or prevention of diseases and disorders that can be effectively treated by PAN BET inhibitors. In one or more embodiments, the PAN BET inhibitors are soft PAN BET inhibitors. In some embodiments, the compounds disclosed herein are effective drugs in the local treatment of diseases and disorders when the compounds or a pharmaceutical composition comprising the compound (or compounds) is administered locally or topically.

In some embodiments, the derivatives disclosed are based on a pyrrolo pyridine core. Such derivatives may have certain groups at the 4-position of the pyridone ring, as defined herein. Advantageously, the use of the compounds disclosed herein have the potential for avoiding e.g., significant unwanted side effects, significant hormone-related systemic adverse events, and adverse events that may accompany other treatments such as corticosteroids.

In some embodiments, the present disclosure provides surprisingly active compounds in inhibiting all four BET BRDs, with effective potency at e.g., nanomolar concentrations. In some embodiments, the active compounds whilst binding to BET BDI and BET BDII, do not exhibit a high selectivity to BDII. It has further been surprisingly observed that whilst some such compounds may show some selectivity or slight bias for BETH they can function effectively as PAN BET inhibitors binding to and inhibiting both BDI and BDII domains.

In one or more embodiments, the active compounds are administered in a composition or excipient. In some embodiments, they are dissolved; in some embodiments, they are suspended; and, in some embodiments, part is dissolved and part suspended in the composition or excipient. In some embodiments, the majority is suspended and can provide a local reservoir of active compound which dissolves over time. In one or more embodiments, the compounds or compositions containing them are administered topically or locally. In some embodiments, they are applied topically to the skin or mucosa on and/or about an area where there is a disease or disorder. In some embodiments, they are delivered intradermally, e.g., into the epidermis and dermis. In some embodiments, they are delivered transdermally. In some embodiments, they are applied within a body cavity. In some embodiments, they may be applied locally, e.g., into a joint. In some embodiments, they may be applied into and within a larger cavity like the lungs. In some embodiments, they may be applied onto or within a lesion or wound.

In some embodiments, for example, the compounds disclosed herein may hold a key advantage as a potential therapy for neutrophilic dermatoses by not showing a delay in skin healing, which is a critical component for treating diseases, such as autoimmune skin diseases that are characterized by chronic ulcers and blisters which are challenging to treat and can potentially lead to life-threatening infections and life-changing surgical intervention.

In some embodiments for example, the compounds disclosed herein may hold a key advantage as a potential therapy for pigmentation diseases or disorders such as autoimmune skin diseases that are characterized by patches of varying pigmentation like vitiligo and which are challenging to treat. Vitiligo and other pigmentation disorders can cause psychological distress and has the ability to affect a person's outlook and social interactions. Common treatments include camouflage therapy, repigmentation therapy, light therapy and surgery.

For internal diseases and disorders, topical application may include topical application to a body cavity surface, for example within the lung (e.g., by inhalation). In some embodiments, inhalation can be with a nebulizer to deliver the drug in a mist. In some embodiments, inhalation is by an inhaler, which can be dry powder inhaler or a metered dose inhaler. In some embodiments, delivery may be by a puff. In some embodiments, delivery to the lungs involves aerosols. In some embodiments, delivery may be by a liquid bolus. In some embodiments, delivery may be by a spray. In some embodiments, delivery may be a mist.

In addition, the compounds disclosed herein may hold a key advantage as a potential therapy for joint or joint related disorders or diseases, including those which are challenging to treat. Additionally, the compounds disclosed herein may also hold a key advantage as a potential therapy for secondary joint diseases or disorders. And, additionally, the compounds disclosed herein may hold a key advantage as a potential therapy for autoimmune diseases and other diseases that are characterized by fibrosis or scarring, like PF and which are challenging to treat.

In some embodiments, an immediate release form of a composition comprising a BETi (e.g., BETi1) is administered to treat a joint or joint related disorder or disease e.g., by local application, or, alternatively, to treat fibrosis or respiratory related disorder or disease. In one or more embodiments, the immediate release form, if comprising suspended particles, will provide for a release over time as the suspended drug particles dissolve. In some embodiments, the immediate release form comprises a solution of the BETi. In some embodiments, the immediate release form comprises a suspension of the BETi or as a micronized or nanoparticle suspension of the BETi. In one or more embodiments, where a formulation with suspended drug particles is provided for immediate release, any solubilized drug should be immediately available and the suspended particles dissolve, e.g., within the joint synovial fluid. In one or more embodiments, the smaller particles will dissolve more quickly than the larger particles, which may provide a reservoir and prolong the period that active drug is available in the joint. In some embodiments, the immediate release form comprises the BETi partly in solution and partly suspended. In some embodiments, a suspended or partly suspended form administered locally into a confined area (e.g., into a joint) may form a reservoir from which, over time, active compound goes into solution providing a therapeutic effect for a prolonged period of time when compared to a solution. In some embodiments, an extended or sustained release form of a composition comprising a BETi (e.g., BETi1) is administered, e.g., to treat fibrosis or respiratory related disorder or disease. In some embodiments, a combination of immediate and extended or sustained release forms is provided. In one or more embodiments, the release form is delivered using an inhalation device, such as a nebulizer, a metered dose inhaler (MDI) and a dry powder inhaler (DPI) or an Aerolizer™ inhaler where the BETi, (e.g., BETi1) may be in a form of capsule. In some embodiments, the release form comprises a suspension of BETi. In some embodiments, the release form comprises a solution of the BETi or as a micronized or nanoparticle suspension of the BETi. In some embodiments, the release form comprises the BETi partly in solution and partly suspended. In some embodiments, the majority of the BETi is in solution (e.g., about 55%, about 60%, about 65%, about 70%, about 75% about 80%, about 85%, about 90%, about 95% or more). In some embodiments, the majority of the BETi is suspended (e.g., about 55%, about 60%, about 65%, about 70%, about 75% about 80%, about 85%, about 90%, about 95% or more). In some embodiments, a sustained release form of a composition comprising a BETi (e.g., BETi1) is administered to treat a joint or joint related disorder or disease or a secondary joint disease or disorder e.g., by local application, or, alternatively, is administered to treat a respiratory or respiratory related disorder e.g., by local application such as inhalation. In some embodiments, the sustained release form comprises BETi loaded in inhalable liposomes, endogenous lipids-based formulations or drug-lipid conjugates. In some embodiments, the suspended particles provide for a sustained release following an initial release. In some embodiments, the sustained release form comprises a particle or encapsulated suspension of the BETi. In some embodiments, the sustained release form comprises the BETi partly suspended to provide for delayed release and partly in solution to provide for an initial release. As will be appreciated by one skilled in the art, the proportion of suspended to dissolved will depend at least in part on the vehicle or carrier and the amount of the BETi.

In some embodiments, a sustained release form of a composition comprising BETi (e.g., BETi1) is administered to treat a joint or joint related disorder or disease e.g., by local application or administration. In some embodiments, the local administration of a composition comprising BETi (e.g., BETi1) is via, for example, injection into the intra-articular space, peri-articular space, soft tissues, lesions, epidural space, perineural space, or the foramenal space at or near the site of a subject's pain. In some embodiments, the formulation (e.g., composition) additionally contains an immediate release component. In some embodiments, a sustained release form of a composition comprising a BETi, e.g., BETi1 is administered (e.g., by single injection or as sequential injections) into an intra-articular space for the treatment of a joint or joint related disorder or disease, such as arthritis, bursitis, Ehlers-Danlos syndrome, epicondylitis, Felty Syndrome, gouty arthritis, psoriatic arthritis, osteoarthritis, rheumatoid arthritis, Still's disease, tenosynovitis, synovitis, Sjögren's Syndrome, lyme disease, Whipple disease, bone cancer, lupus or other autoimmune joint disorders. In some embodiments, a sustained release form of a composition comprising a BETi, e.g., BETi1 is administered (e.g., by single injection or as sequential injections) into soft tissues or lesions for the treatment of inflammatory disorders, such as rheumatoid arthritis. In some embodiments, a sustained (or extended) release form of a composition comprising a BETi, e.g., BETi1 is administered (e.g., by single inhalation or as sequential inhalations) into the respiratory system for the treatment of pulmonary fibrosis or respiratory system related disorder or disease.

In some embodiments there is provided a composition, e.g., an intra-articular composition, comprising a BETi (e.g., BETi1). In some embodiments, the intra-articular composition is encapsulated in a poly(lactic-co-glycolic acid) (PLGA) microsphere. In some embodiments, the PLGA microsphere entraps BETi (e.g., BETi1), and provides a slow-release thereof upon degradation of the PLGA within the joint. In some embodiments, the ratio of lactic acid to glycolic acid is about 80:20 to about 60:40 or is about 75:25 of lactic acid to glycolic acid for the PLGA microsphere. In some embodiments, the drug load (i.e., BETi load) in the microspheres is between the range of about 10% to about 80%. In some embodiments, the drug load in the microspheres is about 25% w/w. In some embodiments, the composition comprises a hyaluronic acid. In some embodiments, the hyaluronic acid is cross-linked to a polymer in order to increase viscosity and slow down degradation within the joint. In some embodiments, there is provided a method of restoring visco-elasticity to the synovial fluid. In other embodiments, BETi (e.g., BETi1) is entrapped or suspended within the cross-linked hyaluronic acid polymer in order to provide a sustained release. In some embodiments, BETi formulations for immediate and/or sustained (extended) release are provided in different delivery forms. In some embodiments, BETi formulations for extended release are deposited without immediately penetrating allowing delivery over the course of one, two, three, four, five, six days or over the course of one, two, three or four weeks or over the course of one, two, three or four months. In some embodiments, BETi formulations are provided formulated in part for immediate release and in part for sustained (extended) release so that the formulation can provide the dual benefits of an initial effect followed by extended release for a continued and prolonged effect, thereby reducing the dosing frequency whilst potentially increasing patient compliance. There may be additionally a benefit of reducing or ameliorating peaks and troughs that can be associated with immediate release formulations and thereby any possible related side effects.

In some embodiments, BETi formulations may be provided in one or more of pMDI (puffer inhalers), Dry-Powder Inhalers (DPI), SMI (mist inhalers) or nebulizers. Such devices are designed to be administered daily or at such other intervals as are prescribed. In some embodiments, BETi formulations are designed for extended-release, and are capable of depositing BETi into the lung (lumen) and slowly releasing therein. In some embodiments, the Particle Size Distribution (PSD) of BETi in formulations for extended-release, impact the penetration of BETi deposited in the lung (lumen). In some embodiments, BETi formulations are provided in liposomes, which are suitable for extended-release. In some embodiments, BETi formulations provided in DPIs are suitable for extended-release, for example where the BETi is coated with one or more layers or is provided incorporated in small particles (e.g., microspheres or nanospheres) from which BETi is slowly released. Such coatings or particles will comprise materials that can dissolve in the lung and are generally non-irritant.

In some embodiments the BETi is provided in a lyophilized formulation. In some embodiments, a lyophilized formulation comprises or consists of one or more of a sugar (e.g., dextran, sucrose or trehalose) in water and optionally a surfactant (e.g., polysorbate 80 or polysorbate 20) or a polar solvent (e.g., propylene glycol or glycerin) or cyclodextrin or a derivative thereof (e.g., 2HP-βCD or betadex sulfobutyl ethyl sodium).

In some embodiments, formulations suitable for DPIs comprise or consist of spray-dried lactose or lactose monohydrate about 50-200 μm with up to about 5-10% loading of the BETi e.g., BETi1. In some embodiments, superfine grades are used of about d50<20 μm. In some embodiments, DPI formulations comprise or consist of lactose—mannitol mixtures of about 10-90-90-10% w/w with up to about 5-10% loading of the BETi e.g., BETi1. In some embodiments, DPI formulations comprise or consist of lactose—sucrose mixtures of about 10-90-90-10% w/w with up to about 5-10% loading of the BETi e.g., BETi1. In some embodiments, Dry-Powder Inhalers formulations comprise or consist of silicone dioxide (e.g., about 0.5%), magnesium stearate (e.g., about 5%), lactose monohydrate (e.g., about >90%) with up to about 5% BETi, e.g., BETi1 loading.

In some embodiments, formulations suitable for capsules for DPIs comprise or consist of crystalline lactose of e.g., about 50-200 μm, or about 50-20 μm, or about <20 μm, HPMC (hydroxy propyl methyl cellulose) and optionally magnesium stearate about <20 μm with up to about 5-10% loading of the BETi e.g., BETi1. In some embodiments, formulations suitable for capsules for DPIs comprise or consist of crystalline lactose e.g., as aforesaid, HPMC and optionally carrageenan about <20-200 μm with up to about 5-10% loading of the BETi e.g., BETi1. In some embodiments, formulations suitable for capsules for DPIs comprise or consist of crystalline lactose e.g., as aforesaid, HPMC and optionally silicon dioxide about <20-200 μm with up to about 5-10% loading of the BETi e.g., BETi1. In some embodiments, formulations suitable for capsules for DPIs comprise or consist of crystalline lactose e.g., as aforesaid, mannitol about <20 μm and HPMC, with up to about 5-10% loading of the BETi e.g., BETi1. In some embodiments, formulations suitable for capsules for DPIs comprise or consist of crystalline lactose e.g., as aforesaid, sucrose, trehalose, or another sugar of about <20 μmol and about <20 μm and HPMC, with up to about 5-10% loading of the BETi e.g., BETi1. In some embodiments, formulations suitable for capsules for DPIs comprise or consist of crystalline lactose of e.g., about 50-200 μm, mannitol of about <20 μm and HPMC, with up to about 5-10% loading of the BETi e.g., BETi1.

In some embodiments, a liposome formulation comprises or consists of phospholipids (e.g., about 5%) in water. In some embodiments, a liposome formulation comprises or consists of phospholipids (e.g., about 2%) and cholesterol (e.g., about 2%) in water. In some embodiments, a liposome formulation comprises or consists of phosphatidylcholine (e.g., about 0.5%) in water.

In some embodiments, a liposome formulation comprises or consists of phospholipids or derivatives thereof, water or saline, soybean oil and optionally and a polar solvent (e.g., propylene glycol or glycerin). In some embodiments, a liposome formulation comprises or consists of phospholipids or derivatives thereof, water or saline, soybean oil and cholesterol. In some embodiments, a liposome formulation comprises or consists of phospholipids or derivatives thereof, water or saline, soybean oil, cholesterol and a polar solvent (e.g., propylene glycol or glycerin). In some embodiments, a liposome formulation comprises or consists of phosphatidylcholine, soybean oil, water or saline and optionally a polar solvent (e.g., propylene glycol or glycerin).

In one or more embodiments the ingredients by way of illustration may be provided in the following amounts or ranges, dextran (about 0.1% to about 10% or about 0.1% to about 1%), sugars (about 0.1% to about 5%) short chain alcohols e.g., ethanol (about 0.1% to about 40% or about 0.1% to about 5%), glycerin or glycols (about 0.1% to about 5% or about 0.1% to about 2.5%), polymeric agents such as carbomers or carrageenan (about 0.05% to about 30% or about 0.10% to about 0.5%), surfactants (about 0.10% to about 5% or about 0.10% to about 2%), and poloxamers (about 0.1% to about 2% or about 0.2% to about 1%). When oils or triglycerides are present, they may be about 0.1% to about 10%. For liposomes the composition comprises phospholipids and optionally cholesterol which may each be about 0.3% to about 8% or about 0.5% to about 5%.

In some embodiments, the formulation is provided as micellar solutions or suspensions. In some embodiments, micellar solutions comprise or consist of water (e.g., about 90%), natural oils (e.g., canola, sesame, soybean, grape seed) (e.g., about 3%), hexelene glycol (e.g., about 2%), propylene glycol (e.g., about 5%) and a poloxamer e.g., poloxamer 182/184 (e.g., about 0.5%).

In some embodiments, micellar solutions or suspensions comprise or consist of water, natural oils (e.g., canola, sesame, soybean, grape seed), at least one polar solvent (e.g., hexelene glycol and/or propylene glycol) and optionally a poloxamer (e.g., poloxamer 182/184). In some embodiments, micellar solutions or suspensions comprise or consist of water, natural oils (e.g., canola, sesame, soybean, grape seed), a poloxamer (e.g., poloxamer 182/184) and optionally a surfactant (e.g., polysorbate 80 or polysorbate 20). In some embodiments, micellar solutions or suspensions comprise or consist of water, natural oils (e.g., canola, sesame, soybean, grape seed), and a surfactant (e.g., polysorbate 80 or polysorbate 20). In some embodiments, micellar solutions or suspensions comprise or consist of water, at least one polar solvent (e.g., hexelene glycol and/or propylene glycol), a poloxamer (e.g., poloxamer 182/184), medium chain triglycerides (MCT), MCT derivatives or coconut oil. In some embodiments, micellar solutions or suspensions comprise or consist of water, a poloxamer (e.g., poloxamer 182/184), medium chain triglycerides (MCT), MCT derivatives or coconut oil and optionally at least one surfactant (e.g., polysorbate 80 or polysorbate 20). In some embodiments, micellar solutions or suspensions comprise or consist of water, at least one surfactant (e.g., polysorbate 80 or polysorbate 20), medium chain triglycerides (MCT), MCT derivatives or coconut oil and optionally a poloxamer (e.g., poloxamer 182/184). In some embodiments, micellar solutions or suspensions comprise or consist of water, a poloxamer (e.g., poloxamer 182/184), medium chain triglycerides (MCT), MCT derivatives or coconut oil and optionally at least one polar solvent (e.g., hexelene glycol and/or propylene glycol).

In some embodiments, the active compounds whilst binding to BET BDI and BET BDII, do not exhibit a high selectivity to BDII. It has further been surprisingly observed that whilst some such compounds may show some selectivity or slight bias for BETII they can function effectively as PAN BET inhibitors binding to and inhibiting both BDI and BDII domains.

In some embodiments, it has been found that the compounds and compositions of this disclosure are surprisingly active in inhibiting BET BRD I and II. In some embodiments, the compounds disclosed herein show an effective potency at low concentrations, including at nanomolar concentrations. It has further been found that surprisingly such compounds with higher liver clearance rates can be effective when applied topically, such as to a body cavity surface (e.g., within the lung, for example, by inhalation or, alternatively, locally, e.g., into a joint. Higher clearance rates help maintain a low plasma concentration of active compound as it passes into the blood, e.g., through the lung surface or out of the joint or transdermally. It has additionally been found that surprisingly such compounds are stable under topical or local application. It has additionally been found that surprisingly such compounds with lower plasma or systemic stability can be effective when administered or applied topically (e.g., by inhalation) or as an intra-articular composition. Moreover, it has further been found that surprisingly such compounds with higher liver clearance rates and lower plasma or systemic stability can be effective when administered or applied, for example, topically, such as to a body cavity surface, or, alternatively, as an intra-articular injection. In one or more embodiments, the compounds when applied topically to the skin or a body cavity or to a mucosal surface (e.g., within the lung) penetrate intradermally, within the cavity surface, or intramucosally, respectively, but have no or minimal transdermal penetration. In which case, the compounds will be below the therapeutic window and may have no significant systemic effects or unwanted systemic side effects. In one or more embodiments, the compounds when applied topically to the skin, to a body cavity, or to a mucosal surface (e.g., within the lung) penetrate transdermally, transmucosally, or through the tissue, so in addition to any intradermal, tissue, or mucosal effect they can have significant systemic effects or unwanted systemic side effects. In some embodiments, transdermal or tissue penetration can be within the therapeutic window and/or at a level that they can induce unwanted side effects. In some embodiments, the composition when administered, for example, as an intra-articular injection has no significant systemic effects or unwanted systemic side effects. By selecting, in some embodiments, compounds that have a higher liver clearance rate and/or a lower plasma stability level, any systemic presence may be eliminated quickly, and any systemic effects are minimized or avoided. By selecting, in one or more embodiments, compounds that can penetrate through the skin, a body cavity surface (e.g., within the lung), or mucosa and which additionally have a lower liver clearance rate and/or a higher plasma stability level, any systemic presence may be eliminated more slowly and, in some embodiments, such compounds can have a systemic therapeutic effect to compliment any dermal, tissue, or mucosal therapeutic effect.

In some embodiments, one or more inactive ingredients are added to the above formulations, for example at the following concentrations or levels, antioxidants (about € 2% w/w), pH buffers (about pH 6-8), preservatives (e.g., if multidose) e.g., benzyl alcohol (about <1% w/w), chelating agents (about <2% w/w). In some embodiments, one or more cosolvents are provided.

In some embodiments, the particle-size distribution for the BETi e.g., BETi1 in suspension or in dry powder inhalers (“DPIs”) is, e.g., about 2-3 μm. In some embodiments, the concentration of the BETi e.g., BETi1 in DPI formulations is up to about 10% w/w of dry weight. In some embodiments, coarse and fine lactose are combined to modify the flow of the BETi e.g., BETi1 in DPIs.

In some embodiments, the concentration of the BETi e.g., BETi1 in formulations can be between about 0.001% to about 1% w/v. In some embodiments, the concentration of the BETi e.g., BETi1 in formulations can be between about 0.001% to about 0.1% w/v.

In some embodiments, the concentration of the BETi e.g., BETi1 in formulations other than DPIs can be up to about 1 mg/mL or about 1% w/v. In some embodiments, depending on the route, formulation, and type of application, it can be higher, say up to about 1.5%, or up to about 2%, or up to about 3%, or up to about 6%. In some embodiments, excipients for inhaled use have an established history in respiratory use.

In some embodiments, extended-release formulations comprise or consist of one or more of mucoadhesives, SiO₂, celluloses, sugars, diatomaceous earths (e.g., bentonite), or calcium carbonate. In some embodiments, sugars (depending on the length of the carbon chain) are used to generate suspension. In some embodiments, sugars (e.g., lactose monohydrate) are used in dry powder inhalers. In some embodiments, tablet coating agents (e.g., povidone e.g., about 0.5%) are used to slow down the release of the BETi e.g., BETi1.

It is reported that aerosol particles can respond to changes in the surrounding environmental conditions on timescales comparable to inhalation/exhalation times. Aerosols formed from hygroscopic components (e.g., saline) can increase significantly in size when inhaled whereas, insoluble drug particles may only absorb molecular layers of water. It is suggested that inhaled particles smaller than 1 m in diameter can respond quickly to the surrounding moisture content, except when they contain surfactants or are crystalline or amorphous particles, which can delay absorption of moisture. Inhaled particles larger than 1 m in diameter are said to absorb water much more slowly, nevertheless, the moisture content acquired can influence the disposition of the particle when deposited. Thus, the microphysical processes occurring in the aerosol phase can be in one or more embodiments be regulated or controlled in part, by selecting the composition and size of the aerosol particles, e.g., to delay absorption or to enhance dissolution.

In some embodiments, the compounds disclosed herein are effective therapeutically by administration topically, such as to a body cavity surface (e.g., by inhalation), or, alternatively, by administration using an inhaler. In some embodiments, the body cavity surface can be mucosal. In some embodiments, the body cavity surface can be epithelial. In some embodiments, the body cavity surface can be the surface of the air sacs in the lungs. In some embodiments, the body cavity surface can be the surface of the interstitium. In some embodiments, the body cavity surface can be the surface of respiratory epithelium or parenchyma. In some embodiments, e.g., when applied topically, systemic penetration is low and/or below a therapeutic window. In other embodiments, e.g., when applied topically, systemic penetration may be sufficient for the compounds to have a systemic therapeutic effect in addition to any topical effect. In some embodiments, the administration is designed to be intradermal. In some embodiments, the administration is designed to be transdermal. In some embodiments, the administration is designed to be intramucosal or intratissue within the epithelial layer. In some embodiments, the administration is designed to be transmucosal or transepithelial.

Topical delivery can be by application of the compound or a pharmaceutical composition comprising the compound to the skin, epithelial, or mucosal surface of a subject, including internal surfaces of body cavities, such as the rectum, gastrointestinal tract, vagina, air passageways, and lungs. Potential half-life may be indicated by factors like the liver clearance rate and/or plasma stability. Compounds which have both the properties of a higher liver clearance rate, and a lower plasma stability may, in one or more embodiments, be more effective topically or locally. Compounds which have either of the properties of a higher liver clearance rate, or a lower plasma stability may, in one or more embodiments, be more effective topically.

The potential to be effective against one or more disorders or diseases described herein may, in one or more embodiments, be illustrated by the ability of the compounds to modulate or reduce biomarkers known or implicated, for example, in an inflammatory or immune response. Non limiting examples, include the reduction in the levels of one or both of IL-17 and IL-22; and/or reduction in the levels of one or more of TH17 cytokine biomarkers including IL-1β, IL-17, IL-6, IL-36, TNFα and CXC motif chemokine ligand 10 (LP-10); and/or reduction in the levels of one or more of TH2 cytokine biomarkers IL-4, 1L-13, and IL-31.

In some embodiments, the compounds disclosed herein are effective therapeutically by administration as an intra-articular injection, or, alternatively, using an inhaler. In some embodiments, an immediate, a controlled, or a sustained release preparation or formulation comprising a BETi is administered as one or more intra-articular injections, or, alternatively, as a dry powder. In some embodiments, a combination of an immediate release form and a sustained release form of a BETi (e.g., BETi1) is administered (e.g., by single injection or as sequential injections) into an intra-articular space or into soft tissues to slow, arrest, reverse or otherwise inhibit structural damage to tissues associated with progressive disease such as, for example, the damage to cartilage such as associated with progression of osteoarthritis. In some embodiments, the combination form of BETi can be administered (e.g., by single inhalation or as sequential inhalations) into the respiratory system or into the lung to slow, arrest, reverse or otherwise inhibit structural damage to tissues associated with progressive disease such as, for example, the damage to the lung associated with, for example, progression of PF. In some embodiments, the formulations disclosed herein can achieve initial relief of the acute symptoms (e.g., inflammation) of the joint or joint related diseases or conditions and additionally provide a sustained or long term therapy (e.g., slowing, arresting, reversing, or otherwise inhibiting structural damage to tissues associated with progressive disease), while avoiding long term systemic side effects associated with corticosteroids (e.g., triamcinolone, dexamethasone) administration. In some embodiments, the formulations disclosed herein can achieve initial relief of the acute symptoms (e.g., inflammation) of the lung or respiratory system related diseases or conditions and additionally provide a sustained or long term therapy (e.g., slowing, arresting, reversing or otherwise inhibiting structural damage to tissues associated with progressive disease), while avoiding long term systemic side effects associated with corticosteroids (e.g., prednisone) administration.

In some embodiments, initial relief may be immediate. In some embodiments, initial relief may commence or be experienced soon after treatment has commenced, for example, after about half a day, about a day, about 2 days, about 3 days, about 4 days or about 5 days. In some embodiments, once commenced, the relief experienced can increase or improve, and, in some embodiments, can after a period of time reach a steady state. Multiple factors can affect the presence and therapeutic effectiveness of the active compound, including, if suspended, its rate of dissolution locally (e.g., in a joint into the synovial fluid), its rate of passing into the blood, its clearance, e.g., by the liver and/or kidneys and its stability/breakdown in plasma.

In one or more embodiments, the compound is more soluble in body fluids (e.g., the synovial fluid) than in the formulation. In one or more embodiments, increasing particle size may provide a depot effect providing for a longer residence of drug locally e.g., in a joint as the smaller particles may dissolve and clear faster.

In some embodiments, the sustained release formulation is a suspension of a BETi compound (or two or more BETis) in a vehicle or carrier. In some embodiments, the suspension comprises two or more average particle sizes of the BETi. In some embodiments, the smaller particles may release at a higher rate than the larger particles. In some embodiments, at least a part of the particles are coated to facilitate a slower release rate. In some embodiments the BETi is/are encapsulated, e.g., in microparticles or microspheres.

In some embodiments, there is an improvement in at least one symptom of a joint disorder or disease upon intra-arterial or intra-venous application of about 0.05% to about 15%, such as about 0.01% to about 10%, about 0.1% to about 10%, about 0.1% to about 5%, about 1% to about 10%, or about 1% to about 5% of the BETi by weight of the composition or by weight to volume of the composition. In some embodiments, there is an improvement in at least one symptom of a respiratory system disorder or disease upon nasal or intra-pulmonary application of about 0.001% to about 10%, such as about 0.001% to about 1%, about 0.01% to about 1%, or about 0.1% to about 1% of the BETi by weight of the composition or by weight to volume of the composition. Unless specified otherwise, an amount expressed in terms of weight (whether absolute weight or relative to another weight or a volume or as a percentage) of a BETi or of “at least one compound, tautomer, stereoisomer, pharmaceutically acceptable salt, hydrate, and/or deuterated derivative” is based on the weight of the free base of the BETi or compound, respectively.

In some embodiments, there is an improvement in at least one symptom of the joint disorder or disease upon local application within the joint of 0.1% to about 15% of the BETi or about 0.2% to about 10% by weight of the composition or by weight to volume of the composition. In some embodiments the BETi is about 0.1%, or about 0.25%, or about 0.5%, or about 0.75%, or about 1%, or about 1.25%, or about 1.5% or about 1.75%, or about 2%, or about 2.25%, or about 2.5%, or about 2.75%, or about 3%, or about 3.25%, or about 3.5%, or about 3.75%, or about 4%, or about 4.25%, or about 4.5% or about 4.75%, or about 5%, or about 5.5%, or about 6%, or about 6.5%, or about 7%, or about 7.5%, or about 8%, or about 8.5%, or about 9%, or about 9.5%, or about 10%, or about 11%, or about 12%, or about 13%, or about 15%, or about 15% by weight of the composition or by weight to volume of the composition. In some embodiments application of the BETi is e.g., inter-articular and for immediate release and may be administered in a single dose of about 1 mg/vial to about 750 mg/vial, or about 2 mg/vial to about 500 mg/vial, wherein a vial is about 5 ml or about 2.5 ml or about 1 ml or about 0.5 ml depending on the size of the joint and the age and size of the subject. In some embodiments the BETi is inter-articular and for immediate release and may be administered in a single dose of about 1 mg/vial, or about 2 mg/vial, or about 3 mg/vial, or about 4 mg/vial, or about 5 mg/vial, or about 6 mg/vial, or about 7 mg/vial, or about 8 mg/vial, or about 9 mg/vial, or about 10 mg/vial, or about 25 mg/vial, or about 50 mg/vial, or about 75 mg/vial, or about 100 mg/vial, or about 150 mg/vial, or about 200 mg/vial, or about 250 mg/vial, or about 300 mg/vial, or about 350 mg/vial, or about 400 mg/vial, or about 450 mg/vial, or about 500 mg/vial, or about 550 mg/vial, or about 600 mg/vial, or about 650 mg/vial, or about 700 mg/vial, or about 750 mg/vial. In some embodiments application of the BETi is inter-articular and for sustained release and may be administered in a single dose of about 1 mg/vial to about 350 mg/vial, or about 2 mg/vial to about 250 mg/vial, or about 10 mg/vial to about 150 mg/vial, or about 10 mg/vial to about 100 mg/vial, wherein a vial is about 5 ml, or about 2.5 ml or about 1 ml, or about 0.5 ml depending on the size of the joint and the age and size of the subject. In some embodiments the BETi is for sustained release and may be administered in a single dose of about 1 mg/vial, or about 2 mg/vial, or about 3 mg/vial, or about 4 mg/vial, or about 5 mg/vial, or about 6 mg/vial, or about 7 mg/vial, or about 8 mg/vial, or about 9 mg/vial, or about 10 mg/vial, or about 25 mg/vial, or about 50 mg/vial, or about 75 mg/vial, or about 100 mg/vial, or about 150 mg/vial, or about 200 mg/vial, or about 250 mg/vial. In some embodiments, where the composition provides an immediate or initial dose and a sustained release dose, the composition may be administered in a single dose of about 1 mg/vial to about 750 mg/vial, or about 2 mg/vial to about 500 mg/vial, or about 2 mg/vial to about 250 mg/vial, or about 10 mg/vial to about 150 mg/vial, or about 10 mg/vial to about 125 mg/vial. wherein a vial is about 5 ml, or about 2.5 ml or about 1 ml, or about 0.5 ml depending on the size of the joint and the age and size of the subject.

In some embodiments, there is an improvement in at least one symptom of the respiratory system disorder or disease upon topical application of 0.001% to about 1% or about 0.01% to about 1% or of about 0.01% to about 1% or of about 0.01% to about 10% of the BETi by weight of the composition or by weight to volume of the composition. In some embodiments, the BETi is about 0.001%, or about or about 0.002%, or about 0.003%, or about 0.004%, or about 0.005%, or about 0.006% or about 0.007%, or about 0.008%, or about 0.009%, or about 0.01%, or about 0.02%, or about 0.03%, or about 0.04%, or about 0.05% or about 0.06% or about 0.07%, or about 0.08%, or about 0.09% or about 0.1% about 0.2%, or about 0.25%, or about 0.3%, or about 0.4%, or about 0.5%, or about 0.6% or about 0.7%, or about 0.8%, or about 0.9%, or about 1% or about 1.5% about 2%, or about 2.5%, or about 3%, or about 4%, or about 5%, or about 6% or about 7%, or about 8%, or about 9%, or about 10% by weight of the composition or by weight to volume of the composition. In some embodiments, application of the BETi is e.g., inhaled and for immediate release and may be administered in a single dose of about 0.001 mg/vial to about 750 mg/vial, or about 0.02 mg/vial to about 50 mg/vial, or about 0.06 mg/vial to about 3 mg/vial, about 0.001 mg/vial to about 100 mg/vial, or about 0.2 mg/vial to about 50 mg/vial, or about 0.6 mg/vial to about 3 mg/vial wherein a vial is about 100 ml, or about 50 ml, or is about 10 ml or about 5 ml or about 2.5 ml or about 1 ml depending on the size of the lung and the age and size of the subject. In some embodiments, the BETi is inhaled and for immediate release and may be administered in a single dose of about 0.005 mg/vial, or about 0.006 mg/vial or about 0.007 mg/vial, or about 0.008 mg/vial, or about 0.009 mg/vial, or about 0.01 mg/vial, or about 0.02 mg/vial, or about 0.03 mg/vial, or about 0.04 mg/vial, or about 0.05 mg/vial or about 0.06 mg/vial, or about 0.07 mg/vial, or about 0.08 mg/vial, or about 0.09 mg/vial, or about 0.1 mg/vial, or about 0.2 mg/vial, or about 0.25 mg/vial, or about 0.3 mg/vial, or about 0.4 mg/vial, or about 0.5 mg/vial, or about 0.6 mg/vial or about 0.7 mg/vial, or about 0.8 mg/vial, or about 0.9 mg/vial, or about 1 mg/vial, or about 2 mg/vial, or about 3 mg/vial, or about 4 mg/vial, or about 5 mg/vial, or about 6 mg/vial, or about 7 mg/vial, or about 8 mg/vial, or about 9 mg/vial, or about 10 mg/vial, or about 25 mg/vial, or about 50 mg/vial, or about 75 mg/vial, or about 100 mg/vial, or about 150 mg/vial, or about 200 mg/vial, or about 250 mg/vial, or about 300 mg/vial, or about 350 mg/vial, or about 400 mg/vial, or about 450 mg/vial, or about 500 mg/vial, or about 550 mg/vial, or about 600 mg/vial, or about 650 mg/vial, or about 700 mg/vial, or about 750 mg/vial. In some embodiments, application of the BETi is for sustained release and may be administered in a single dose of about 0.001 mg/vial to about 350 mg/vial, or about 0.002 mg/vial to about 250 mg/vial, wherein a vial is about 100 ml, about 50 ml, or about 10 ml, or about 5 ml or about 2.5 ml or about 1 ml depending on the size of the lung and the age and size of the subject. In some embodiments, the BETi is for sustained release and may be administered in a single dose of about 0.01 mg/vial, or about 0.02 mg/vial, or about 0.03 mg/vial, or about 0.04 mg/vial, or about 0.05 mg/vial, or about 0.06 mg/vial, or about 0.07 mg/vial, or about 0.08 mg/vial, or about 0.09 mg/vial, about 0.1 mg/vial, or about 0.2 mg/vial, or about 0.3 mg/vial, or about 0.4 mg/vial, or about 0.5 mg/vial, or about 0.6 mg/vial, or about 0.7 mg/vial, or about 0.8 mg/vial, or about 0.9 mg/vial, or about 1 mg/vial, or about 2 mg/vial, or about 3 mg/vial, or about 4 mg/vial, or about 5 mg/vial, or about 6 mg/vial, or about 7 mg/vial, or about 8 mg/vial, or about 9 mg/vial, or about 10 mg/vial, or about 25 mg/vial, or about 50 mg/vial, or about 75 mg/vial, or about 100 mg/vial, or about 150 mg/vial, or about 200 mg/vial, or about 250 mg/vial. In some embodiments, where the composition provides an immediate or initial dose and a sustained release dose, the composition may be administered in a single dose of about 0.01 mg/vial to about 750 mg/vial, or about 0.02 mg/vial to about 500 mg/vial, or about 0.02 mg/vial to about 250 mg/vial, wherein a vial is about 50 ml, or about 25 ml, or about 10 ml, or about 5 ml or about 2.5 ml or about 1 ml, depending on the size of the lung and the age and size of the subject.

In some embodiments, the derivatives of pyrrolo N-methyl-2-pyridone compounds described herein can be surprisingly effective as PAN BET inhibitors, as illustrated by the Examples. In some embodiments, the compounds of the present disclosure effectively act as PAN BET inhibitors and have a high liver clearance rate and/or a low plasma stability. In some embodiments, the compounds of the present disclosure provide PAN BET inhibition and have a lower liver clearance rate and/or some plasma stability.

In some embodiments, the compounds disclosed herein are effective drugs in the local treatment of diseases or disorders, such as joint and joint related diseases and disorders, when the compound or a pharmaceutical composition is administered or applied topically, e.g., to the skin or within a body cavity, e.g., through inhalation to a cavity surface such as the lung aveoli or locally as an intra-articular injection.

In one or more embodiments, the compounds and compositions lack solubility in a range of solvents, carriers, and formulations suitable for topical application and can be suspended and dispersed homogeneously therein. Homogenous suspensions thereof are exemplified herein. In one or more other embodiments, the compounds and compositions are also soluble in a range of other solvents carriers and formulations suitable for topical application. In some embodiments, a protic solvent acts to improve solubility of an active agent. In some embodiments, the delivery of the active agent is improved by the aprotic solvent and/or protic solvent. In some embodiments, there is provided a composition comprising an aprotic polar solvent and/or a protic polar solvent, in which the protic polar solvent can be a short chain alcohol. Advantageously, many of the compounds and compositions described herein are stable, for example in human and animal respiratory systems, in mucosal membranes, in joints and/or in skin. Also, under hydrolytic conditions at a range of pH values various compounds can exhibit stability. Furthermore, formulations of the compounds and compositions may deliver practicable concentrations of the compound into the epidermis and/or dermis of the skin or within a mucosal membrane in therapeutically effective amounts and the compounds are not toxic to skin cells or to mucosal cells. By respiratory system is meant the parts of the body involved in breathing when oxygen and carbon dioxide are exchanged. It includes the nose, mouth, throat (pharynx), voice box (larynx), windpipe (trachea), large airways (bronchi), small airways (bronchioles), and lungs. The lungs are air filled organs comprising airways, and respiratory epithelium or parenchyma that comprises a large number of thin-walled alveoli. Also, under hydrolytic conditions at a range of pH values various compounds can exhibit stability. Furthermore, formulations of the compounds and compositions may deliver practicable concentrations of the compound within a mucosal membrane or tissues of the respiratory tract including the lower respiratory tract, and respiratory epithelium or parenchyma of the lung and/or into the epidermis and/or dermis of the skin in therapeutically effective amounts and the compounds are not toxic to mucosal cells or tissues of the respiratory tract including the lower respiratory tract, and respiratory epithelium or parenchyma of the lung or to skin cells. Compounds and compositions of this disclosure in some embodiments exhibit surprisingly effective clearance by the liver, offering potential use as medicaments with a lower risk of side-effects. In some embodiments, once entering the blood system, they break down more rapidly but are sufficiently stable in the respiratory system, including the lower respiratory tract, and respiratory epithelium or parenchyma of the lung, the mucosa and/or in skin to achieve a therapeutic effect, offering potential use as medicaments for topical administration. Similarly, in some embodiments once entering the blood system, they break down more rapidly but are sufficiently stable in the skin, mucosa, or joints, to achieve a therapeutic effect. Some of the compounds, whilst having some selectivity for BDII over BDI, offer the potential of an improved therapeutic effect locally or topically and a lower risk of side-effects. In one or more embodiments, the PAN BET inhibitor has low, very low, or no significant selectivity for BDII over BD1. In some embodiments, the selectivity for BDII over BDI is, e.g., between about 15 fold to about 1 fold, about 15 fold to about 2 fold, or about 10 fold to about 1 fold, or about 10 fold to about 2 fold, or <50 fold; about <15 fold; about <10 fold; about <8 fold; about <6 fold; about <4 fold; about <2 fold; about <1 fold. In some embodiments, the selectivity for BDII over BDI is less than two-fold. In some embodiments, binding to BD1 and to BDII is similar. In some embodiments, the selectivity for BD1 and for BDII is similar. In some embodiments, the selectivity of for BDII over BDI is less than four-fold. In some embodiments, the selectivity of for BDII over BDI is less than six-fold. In some embodiments, the selectivity of for BDII over BDI is less than eight-fold. In some embodiments, the selectivity of for BDII over BDI is less than ten-fold. In some embodiments, the selectivity for BDII over BDI is between about one-fold and about two-fold. In some embodiments, the selectivity of for BDII over BDI is between about two-fold and about four-fold. In some embodiments, the selectivity of for BDII over BDI is between about four-fold and about six-fold. In some embodiments, the selectivity of for BDII over BDI is between about six-fold and about eight-fold. In some embodiments, the selectivity of for BDII over BDI is between than about eight-fold and about ten-fold. In some embodiments, the selectivity of for BDII over BDI is greater than ten-fold.

BET inhibitors have been suggested as a treatment for very long lists of a multitude of disorders and diseases. Almost all BET inhibitors in development are for oral delivery e.g., for the systemic treatment of cancers. In some embodiments, there is provided compounds that can function as a “soft” PAN BD BET inhibitor. In one or more embodiments a BETi is a “soft drug”. In one or more embodiments a BETi is metabolised on reaching the blood or liver. In one or more embodiments clearance of a BETi within a joint is unpredictable. In one or more embodiments, the clearance and pK of a BETi within a joint is multifactorial, and may be impacted by factors including solubility, dose, particle size, formulation of the active compound, the size of joint, solubility and stability in the joint, and lea\k\age therefrom.

In one or more embodiments, the compounds and compositions disclosed herein can impact positively on diseases and disorders involving multiple, diverse inflammatory cell signaling pathways. In some embodiments, the compounds and compositions disclosed herein can impact positively on autoimmune or autoimmune related diseases and disorders. In some embodiments, the compounds disclosed herein can impact positively on skin diseases and disorders, including diseases or disorders involving lesions wounds, e.g., where the healing process is failing or has failed. In some embodiments the compounds disclosed herein can impact positively on joint or joint related diseases and disorders. In some embodiments, the compounds disclosed herein can impact positively on fibrosis or fibrosis-related diseases and disorders. In some embodiments, the compounds disclosed herein can impact positively on pulmonary or pulmonary related diseases and disorders.

As used herein, “BETi” to be used in the present disclosure include compounds of Formula (I), their tautomers, stereoisomers, pharmaceutically acceptable salts, hydrates, and deuterated derivatives thereof:

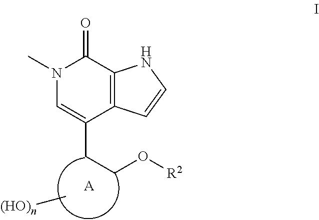

wherein:
ring A is selected from phenyl, N-methyl-2-pyridone, and thiazole;
n is 0 or 1, wherein when A is phenyl, n is 1, when A is N-methyl-2-pyridone,
n is 0, and when A is thiazole, n is 0;
R² is phenyl optionally substituted with 1 to 3 substituents independently selected from hydroxy, halo, C₁-C₆ alkyl, C₃-C₆ cycloalkyl, C₁-C₅ alkyloxy, C₁-C₅ alkylamino, C₁-C₆ fluoroalkyl, C₁-C₅ fluoroalkyloxy, and C₁-C₅ fluoroalkylamino; C₂-C₆ alkyl; and C₃-C₆ cycloalkyl optionally substituted with a substituent selected from C₁-C₆ alkoxy.

In some embodiments, ring A is selected from

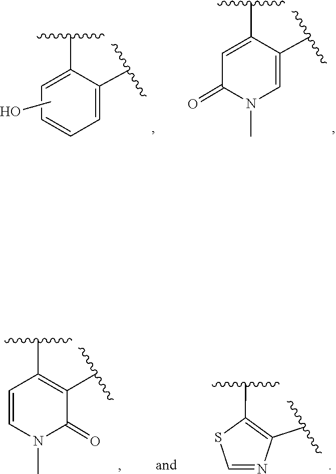

In some embodiments, ring A is selected from

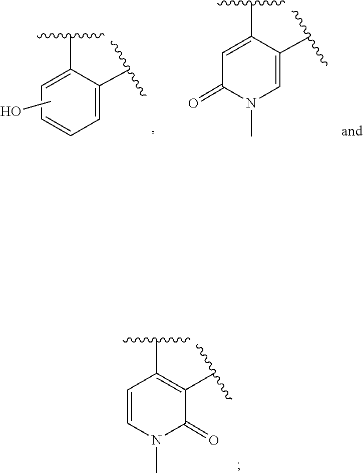

and
R² is phenyl optionally substituted with 1 to 3 substituents independently selected from hydroxy, halo, C₁-C₆ alkyl, C₃-C₆ cycloalkyl, C₁-C₅ alkyloxy, C₁-C₅ alkylamino, C₁-C₆ fluoroalkyl, C₁-C₅ fluoroalkyloxy, and C₁-C₅ fluoroalkylamino.

In some embodiments, the compound of Formula I has the following structure:

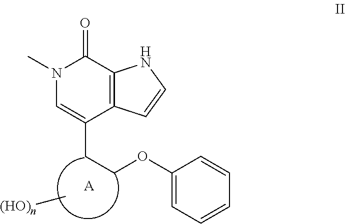

wherein:
ring A is selected from phenyl and N-methyl-2-pyridone; and
n is 0 or 1, wherein when A is phenyl, n is 1, and when A is N-methyl-2-pyridone, n is 0.

In some embodiments, in Formula II, ring A is selected from

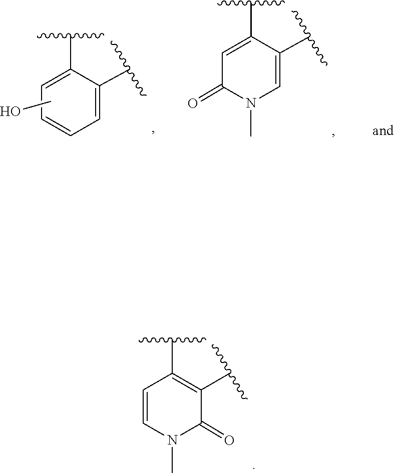

In some embodiments, the compound of Formula I has the following structure:

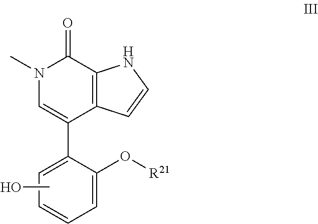

wherein R²¹ is selected from (i) phenyl optionally substituted with 1 to 3 substituents independently selected from hydroxy, halo, C₁-C₆ alkyl, C₃-C₆ cycloalkyl, C₁-C₅ alkyloxy, C₁-C₅ alkylamino, C₁-C₆ fluoroalkyl, C₁-C₅ fluoroalkyloxy, and C₁-C₅ fluoroalkylamino; and (ii) C₃-C₆ cycloalkyl optionally substituted with a substituent selected from C₁-C₆ alkoxy.

In some embodiments, in Formula III, R²¹ is phenyl, cyclobutyl or cyclohexyl optionally substituted with a substituent selected from C₁-C₆ alkoxy.

In some embodiments, in Formula III, R²¹ is phenyl.

In some embodiments, the compound of Formula I is

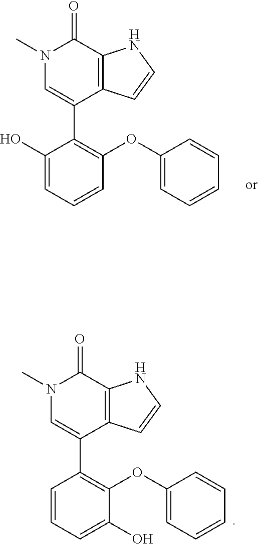 [SMILES: Cn1cc(-c2c(O)cccc2Oc2ccccc2)c2cc[nH]c2c1=O ; Cn1cc(-c2cccc(O)c2Oc2ccccc2)c2cc[nH]c2c1=O]

In some embodiments, the compound of Formula I is

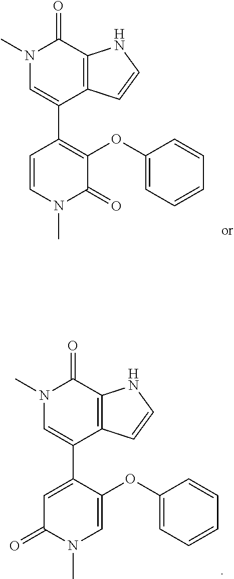 [SMILES: Cn1ccc(-c2cn(C)c(=O)c3[nH]ccc23)c(Oc2ccccc2)c1=O ; Cn1cc(Oc2ccccc2)c(-c2cn(C)c(=O)c3[nH]ccc23)cc1=O]

In some embodiments, the compound of Formula I is

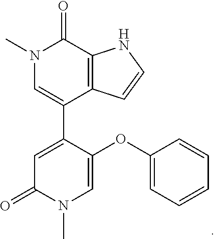 [SMILES: Cn1cc(Oc2ccccc2)c(-c2cn(C)c(=O)c3[nH]ccc23)cc1=O]

In some embodiments, the compound of Formula I is

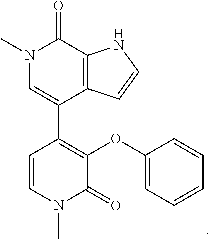 [SMILES: Cn1ccc(-c2cn(C)c(=O)c3[nH]ccc23)c(Oc2ccccc2)c1=O]

In some embodiments, the compound of Formula I is

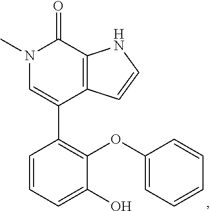 [SMILES: Cn1cc(-c2cccc(O)c2Oc2ccccc2)c2cc[nH]c2c1=O]

which compound is hereinafter referred to as BETi1.

In some embodiments, the compound of Formula I is

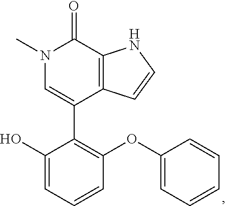 [SMILES: Cn1cc(-c2c(O)cccc2Oc2ccccc2)c2cc[nH]c2c1=O]

In some embodiments, the compound of Formula I is

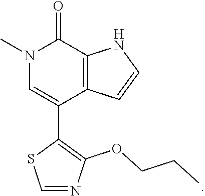 [SMILES: CCCOc1ncsc1-c1cn(C)c(=O)c2[nH]ccc12]

In some embodiments, the compound of Formula I is

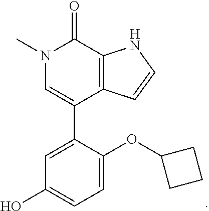 [SMILES: Cn1cc(-c2cc(O)ccc2OC2CCC2)c2cc[nH]c2c1=O]

Tautomers and stereoisomers of compounds of Formula (I)-(III) as well pharmaceutically acceptable salts, hydrates, and deuterated derivatives of any of the foregoing are included herein.

In one or more embodiments, there is provided compounds that are applicable to and can have therapeutic activity to a specific set of diseases and disorders involving cytokine pathways as part of the etiological factors implicated in the manifestation of the symptoms thereof, for example, to a number of neutrophilic dermatoses such as pyoderma gangrenosum (PG), palmoplantar pustulosis (PPP), and generalized pustular psoriasis (GPP). Alternatively, there is provided compounds that are applicable to and can have therapeutic activity, to pigmentation disorders, e.g., vitiligo, and/or pigmentation related disorders. Pigmentation related disorders (e.g. skin disorders) that are similar to vitiligo and may be treated by compounds of the present invention include chemical leukoderma resulting in linear or splotchy white areas of skin, tinea versicolor spots, albinism where there are lower levels of melanin in the skin, and pityriasis alba which results in lighter patches of skin.

In some embodiments, pigmentation disorders may be separated into two primary groups. The first group concerns the appearance of pale or white skin patches, for example, where there is a reduction in melanin, which results in pale patches (hypopigmentation, hypomelanosis) or white skin or white patches (depigmentation, leukoderma). These are typically due to an absence of melanocytes or loss of melanin in the epidermis. In one or more embodiments the usefulness of the BET inhibitors provided herein for the first group is illustrated by the various Examples herein in respect of skin morphology, melanocytes, MMP-9, E-cdherin, TRP-1 expression, melanin content, and gene expression. In some embodiments, this first group may benefit from the BET inhibitors provided herein, without being bound by any theory, by helping to retain, maintain, or preserve the localization the melanocytes at the basal layer and may over time help restore skin color or slow and/or avoid further deterioration of skin color. In one or more embodiments, a pigmentation disorder includes, vitiligo, chemical leukoderma, tinea versicolor spots, albinism, and pityriasis alba. Also, atrophie blanche, Griscelli syndrome, Halo moles, Hermansky-Pudlak syndrome, Hypomelanosis of Ito, Idiopathic guttate hypomelanosis, Leprosy, Leukoderma, Lichen sclerosus, Lupus erythematosus, Morphoea, Mycosis fungoides, Naevus anaemicus, Naevus depigmentosus, Piebaldism, Pityriasis versicolor, Poliosis, Postinflammatory hypopigmentation, Progressive macular hypopigmentation, Tuberous sclerosis (ashleaf spots), and Waardenburg syndrome.

In some embodiments, the second group concerns the appearance of darker skin patches, for example where there is an increase in melanin (hyperpigmentation, hypermelanosis) which can be due to an increased number of pigment cells (melanocytes) or from increased production of melanin. The Examples herein are concerned with preserving melanocytes and pigmentation and reducing loss of pigmentation and it is currently not known how BET inhibitors may impact on this second group where the position is the reverse and there is darkening from increased or concentrated pigmentation. In one or more embodiments the disorder is vitiligo.

In some embodiments, there is provided compounds that are applicable to and can have therapeutic activity for a joint or joint related disorder or disease.

In some embodiments, the joint or joint related disorder or disease is chosen from arthritis, bursitis, Ehlers-Danlos syndrome, Felty Syndrome, epicondylitis, gouty arthritis, psoriatic arthritis, osteoarthritis, rheumatoid arthritis, Still's disease, tenosynovitis, synovitis, Sjögren's Syndrome, lyme disease, Whipple disease, bone cancer, lupus and other autoimmune joint disorders. In some embodiments, the joint or joint related disorder or disease is chosen from arthritis, bursitis, epicondylitis, gouty arthritis, psoriatic arthritis, osteoarthritis, rheumatoid arthritis, tenosynovitis, synovitis, and autoimmune joint disorders. In some embodiments, the joint or joint related disorder or disease is rheumatoid arthritis. Other diseases or disorders treatable topically by the compounds disclosed herein are listed in the detailed description.

The present disclosure provides exemplary BETi that may also provide a new and effective treatment or relief for secondary joint diseases and disorders.

In some embodiments there are provided compounds that are applicable to and can have therapeutic activity to lung diseases and disorders. By therapeutic activity can include preventing, treating, ameliorating, or delaying the progression of the disease or disorder. In some embodiments, there are provided compounds that are applicable to and can have therapeutic activity to fibrotic or scarring diseases or disorders, e.g., PF. In some embodiments, there are provided compounds that are applicable to and can have therapeutic activity to fibrosis related disorders. Fibrosis related disorders (e.g., various ILDs) that are similar to PF and may be characterized by inflammation and/or scarring in the lung tissue, walls of the air sacs or interstitium.

In some embodiments a patient may be diagnosed with “unclassifiable interstitial lung disease” if they do not meet all the criteria to be confidently diagnosed with a specific type of interstitial lung disease or pulmonary fibrosis. This occurs when there are nonspecific or conflicting findings on their CT scans, lung biopsies, or in the clinical course of the disease. Another case when a diagnosis of unclassifiable ILD may occur is when the patient is unable to go through standard diagnostic tests.

In some embodiments there are provided compounds that are applicable to lung diseases or ILDs which may be classified into two groups idiopathic or known (secondary). Idiopathic interstitial pneumonia is the term given to ILDs with an unknown cause. They represent the majority of cases of interstitial lung diseases (up to two-thirds of cases). In some embodiments, the fibrosis or respiratory disorder or disease is pulmonary fibrosis (PF). In some embodiments idiopathic ILDs may include idiopathic pulmonary fibrosis (IPF), desquamative interstitial pneumonia (DIP), acute interstitial pneumonia (AIP): also known as Hamman-Rich syndrome, nonspecific interstitial pneumonia (NSIP), respiratory bronchiolitis-associated interstitial lung disease (RB-ILD), Cryptogenic organizing pneumonia (COP): also known by the older name bronchiolitis obliterans organizing pneumonia (BOOP) or lymphoid interstitial pneumonia (LIP).

In some embodiments secondary ILDs are caused by another condition, like sarcoidosis or lymphangioleiomyomatosis, or ILDs which may be the after-effect of acute respiratory distress syndrome, which occurs in critical illness. In some embodiments secondary ILDs are diseases with a known etiology. In some embodiments, a secondary lung disorder includes connective tissue and autoimmune diseases such as sarcoidosis, rheumatoid arthritis, systemic lupus erythematosus, systemic sclerosis, polymyositis, dermatomyositis or antisynthetase syndrome. In some embodiments secondary ILDs include ILDs induced by inorganic inhaled substances (pneumoconiosis) which may include silicosis, asbestosis, berylliosis, industrial printing chemicals (e.g., carbon black, ink mist). In some embodiments secondary ILDs include ILDs induced by inhaled organic substances (pneumoconiosis) which may include hypersensitivity pneumonitis (extrinsic allergic alveolitis), drug-induced such as antibiotics, chemotherapeutic drugs, antiarrhythmic agents, infection (coronavirus disease), atypical pneumonia, pneumocystis pneumonia (PCP), tuberculosis, Chlamydia trachomatis, respiratory syncytial virus), malignancy (lymphangitic carcinomatosis). ILD's can occur predominately in children due to e.g., diffuse developmental disorders, growth abnormalities deficient alveolarisation, infant conditions of undefined cause, and ILD related to alveolar surfactant region.

In some embodiments, the fibrosis or fibrosis-associated disorder is interstitial lung fibrosis. In some embodiments, there is provided a method for treating a fibrosis or fibrosis-associated disorder that affects other organs or tissues such as the liver, epidermis, endodermis, muscle, tendon, cartilage, heart, pancreas, lung, uterus, nervous system, testis, ovary, adrenal gland, artery, vein, colon, small intestine, biliary tract, or stomach. In some embodiments, the fibrosis or fibrosis-associated disorder is the result of an infection with schistosoma. In some embodiments, the fibrosis or fibrosis-associated disorder is the result of wound healing. In some embodiments, the wound healing results from a surgical incision.

In some embodiments, PF may be separated into six primary groups. The first group is environmental induced (also known as (“-a\k\a”) hypersensitivity pneumonitis) which includes exposure to pollution, mold, animals. The second group is occupational induced (a\k\a pneumoconiosis), which includes exposure to dusts, fibers, fumes or vapors, such as, asbestos, coals, and silica. The third group is drug-induced such as amiodarone, nitrofurantoin, methotrexate and chemotherapy. The fourth group is radiation-induced. The fifth group is autoimmune induced (a\k\a connective tissue disease-related) which is characterized by joint inflammation, skin changes, (particularly fingers and face), dry mouth or eyes, or abnormal blood tests. The last group is idiopathic, which is the most common form of PF and has a high mortality rate within three to five years. In some embodiments, a fibrosis disorder includes PF which is induced by causes such as long-term exposure to certain toxins (e.g. tobacco smoking), secondary effect of other diseases and disorders such as, ILD, viral infections (e.g. COVID-19 and related SARS viruses), and bacterial infections.

In some embodiments, other forms of PF may have a genetic component, like familial pulmonary fibrosis and pulmonary fibrosis associated with Hermansky Pudlak syndrome or dyskeratosis congenita. For example, a mutation in surfactant protein C(SP-C) has been found to exist in some families with a history of pulmonary fibrosis. Autosomal dominant mutations in the TERC or TERT genes, which encode telomerase, have been identified in about 15 percent of PF patients.

Some patients have forms of pulmonary fibrosis that do not fit into any of the known categories of interstitial lung disease or have overlapping features of multiple types of ILD, which is termed “unclassifiable ILD”.

In addition to the potential treatment benefits of the compounds provided herein for pigmentation diseases and disorders such as vitiligo and related pigmentation diseases and disorders, for lesion and wound diseases, e.g., with compromised healing and other similar skin disorders, for joint or joint related diseases or disorders or, for lung diseases and disorders, particularly those involving fibrosis or scarring, e.g., PF and other similar disorders involving fibrosis or scarring, by administering them topically or locally after treatment or prior to onset or relapse of a disease or disorder in a subject with a propensity to develop such a disease or disorder, they can, in some embodiments, have a prophylactic or preventative effect such that the disease or disorder is slower, or it does not develop further, or the disease or disorder does not develop or appears in milder, or less severe forms, or does not appear.

### BRIEF DESCRIPTION OF THE DRAWINGS

FIGS. 1A-H show results for efficacy and tolerance from a murine imiquimod-induced inflammatory model. FIG. 1A shows Psoriasis Index (PASI) for animals treated with different petrolatum-based formulations for 7 consecutive days, at different BETi1 strengths compared to three control arms and two dose tolerance arms and FIG. 2C is a pictorial representation of visual parameters at day 14. FIG. 1B shows the mean body weight of animals treated formulations at different BETi1 strengths and controls. Other parameters tested and shown are behavioral parameters i.e., use of enrichment (FIG. 1D), inflammatory biomarkers (FIG. 1E). FIG. 1F shows modified PASI for animals treated with the different formulations, FIG. 1G shows the mean percent change in modified PASI severity score prior to induction for three of the doses compared to steroid and vehicle and FIG. 1H shows the change in mean percent change in body weight thereof.

FIGS. 2A-I show results for a wound healing animal model study.

FIGS. 3A-K show results for qPCR analysis performed on key pro-inflammatory proteins in ex-vivo human model using Th2 and Th7 stimulation to evaluate two BET inhibitors. FIGS. 3A-C show expression of cytokines relevant to Th17-mediated autoimmune diseases in Th17 induced phenotype in human skin and FIGS. 3D-F show expression of cytokines relevant to Th2-mediated autoimmune diseases. FIG. 3G-3K show a pharmacodynamic comparison between BETi1 and BETia. FIG. 3G shows percent activity of IL4 and IL13 calculated to Th2 stimulated control. FIG. 3H shows percent activity of IL31 and CCL17 calculated to Th2 stimulated control. FIG. 3I shows percent activity of CCL26 and CCL2 calculated to Th2 stimulated control. FIG. 3J shows percent activity of CXCL10, IL17a, and IL22 calculated to Th17 simulated control. FIG. 3K shows percent activity of IL367, CCL20, and serpinB4 calculated to Th17 simulated control.

FIGS. 4A-H show H/E staining of histologic sections of paraffin embedded epidermis in a Reconstituted Human Epidermis (RHE) model mimicking vitiligo, comparing untreated to treated with BETi1 formulations at four doses, BETi vehicle (placebo) and a JAK inhibitor.

FIGS. 5A-B show quantification of MMP-9 and E-cadherin secretion by ELISA from collected culture medium.

FIGS. 6A-G show representative images of Fontana Masson staining of reconstituted human epidermis treated with the test formulations (e.g., BETi1 1%, BETi1 0.1%, BETi1 0.01%, BETi1 0.001%, ruxolotinib cream 1.5%; see also Experimental Method K for the test formulations) and stimulated with a cytokine cocktail to exhibit vitiligo characteristics. It is noted that the percentages, e.g., BETi1 1%, BETi1 0.1%, are percent by weight of the pharmaceutical composition/formulation. FIG. 6A shows untreated and unstimulated control.

FIG. 6B shows stimulated and untreated control. FIG. 6C shows stimulated plus vehicle.

FIG. 6D shows stimulated plus BETi1 1%. FIG. 6E shows stimulated plus BETi1 0.1%.

FIG. 6F shows stimulated plus BETi1 0.01%. FIG. 6G shows stimulated plus BETi1 0.001%.

FIG. 6H shows the melanin content in basal layers of RHE-quantification from FM stained tissue sections.

FIGS. 7A-C show TRP-1 expression in the basal layers of epidermis. FIG. 7A shows representative images of immunostaining of RHE after TRP-1 staining for all test formulations and controls (top to bottom untreated and unstimulated control, stimulated and untreated control, stimulated plus vehicle, 1% BETi1, 0.1% BETi1, BETi1 0.01%, 0.001% BETi1). Three representative images per epidermis are shown. TRP-1 proteins appear in green and nuclei in blue. FIG. 7B and FIG. 7C show in a quantification form the image analysis shown in FIG. 7A of the abundance of TRP-1 in the basal layers of the RHE with or without normalization to the surface of the Region of Interest; ROI respectively.

FIGS. 8A-F show volcano plots which show the significant variations of gene expression in RHE for all test formulations and controls. Datapoints with significant p-values appear at the top of the plot, above the horizontal threshold line fixed at a p-value of 0.05. Genes with fold increase (red dots) or decrease (green dots) lies on both sides of the vertical threshold line. Gene symbols are given.

FIG. 8A shows a volcano plot for untreated and stimulated control vs untreated and unstimulated control.

FIG. 8B shows a volcano plot for stimulated BETi1 vehicle vs untreated and stimulated control.

FIG. 8C shows a volcano plot for stimulated BETi1 1% vs stimulated plus BETi1 vehicle.

FIG. 8D shows a volcano plot for stimulated BETi1 0.1% vs stimulated plus BETi1 vehicle.

FIG. 8E shows a volcano plot for stimulated BETi1 0.01% vs stimulated plus BETi1 vehicle.

FIG. 8F shows a volcano plot for stimulated BETi1 0.001% vs stimulated plus BETi1 vehicle.

FIG. 8G shows a volcano plot for Ruxolitinib 1.5% vs untreated and stimulated control.

FIG. 9 shows the percent change in body weight of mice compared to baseline on days 3-8, 10 and 12 of four doses of BETi1, vehicle (positive control for API) and steroid for treatment intra-articular and IP (systemic).

FIG. 10A and FIG. 10B show paw thickness of treated leg and untreated leg respectively. FIG. 10C shows the delta between paw thickness of untreated versus treated leg where a higher delta indicates a better local treatment.

FIGS. 11A and 11B show the arthritis scoring of treated leg and untreated leg respectively. FIG. 11C shows the arthritis scoring Δ untreated—treated leg where a higher Δ means better local treatment.

FIG. 11D shows a histogram of the mean histopathological score (a.u.) of untreated paw (right paw) versus treated paw for vehicle steroid and two highest doses where a higher difference between the paws means better local treatment.

FIGS. 11E-11L show histology results for control and treatment group animals as further described in Example 2.

FIG. 12 shows hot plate change in withdrawal latency (Sec).

FIG. 13 shows homogeneity of BETi1 suspensions using light microscopy.

FIG. 14A, FIG. 14B, and FIG. 14C show photomicrographs of BETi suspensions at 4 mg/g (0.4%), at 0.4 mg/g (0.04%) and at 0.04 mg/g (0.004%) prepared in NaCMC solutions.

FIG. 15A-D show the mean body weight of animals treated with formulations at different BETi1 strengths (0.06 mg/mL, 0.6 mg/mL, 3 mg/mL) and two controls at baseline, week 1, week 2 and week 3 and percent change in body weight for each week from baseline.

FIG. 16 shows weight of whole lung of animals treated with formulations at the different BETi1 strengths and two controls at week 3.

FIG. 17 shows survival probability of animals treated with formulations at the different BETi1 strengths and two controls at week 3.

FIG. 18 shows hydroxyproline content of lung tissue as measured by colorimetric assay of animals treated with formulations at the different BETi1 strengths and two controls at week 3.

FIG. 19A-J show representative micrographs (three representations for each group at 100× and one representation of each group at 40×) of fixed lung tissue stained with H & E and Masson's Trichrome staining of uninduced (sham) and induced treated with vehicle or BETi1 at three strengths and two controls at week 3 and FIG. 19K shows a histogram of the histopathology alterations scored by Ashcroft scale (modified by Hubner) in lung thereof.

FIGS. 20A-B show synovial concentration (20A) and plasma concentration (20B) of BETi1 in dogs treated with BETi1 formulations comprising 7.5 mg (diamond), 75 mg (square), and 150 mg (triangle) doses of BETi1.

FIGS. 21A-D show representative histology sections of Beagle dog treated with vehicle (21A), 7.5 mg BETi1 (21B), 75 mg BETi1 (21C), and 150 mg BETi1 (21D).

### DETAILED DESCRIPTION OF THE DISCLOSURE

The present disclosure relates to the treatment and/or prophylaxis of a set of diseases and disorders that can be treated using a specific set of inhibitors based on an N-methyl-2-pyridone structures/scaffold (i.e., genus). These derivatives have specific groups attached at position 4 of the pyridone ring. The genus discloses herein and the compounds falling within this genus (i.e., the compounds of the present disclosure) were found to be surprisingly effective when comprised in a composition and administered applied topically or locally. The experimental data—demonstrates that they can be effective in relation to lesions and wounds, inflamatory diseases and disorders, autoimmune diseases and disorders, pigmentation diseases and disorders, joint diseases and disorders, fibrosis diseases and disorders and other diseases and disorders—as provided by various models and studies described herein including in relation to wounds, inflammation, cytokines, biomarkers, genetic pathways, vitiligo, arthritis, fibrosis and PF.

The potential of the compounds to treat, reduce or alleviate scarring and reduce swelling is demonstrated herein. In addition, the potential of the compounds to treat, reduce or alleviate vitiligo and similar disorders (e.g., pigmentation related disorders) is demonstrated herein. In addition, the potential of the compounds disclosed herein to treat a joint or joint related disorders and diseases is demonstrated herein. The potential of the compounds to treat, reduce or alleviate PF and similar disorders (e.g., fibrosis and scarring related disorders) is demonstrated herein.

The pathology of vitiligo results from a selective disappearance of melanocytes from the basal layer of the epidermis, a phenomenon called melanocytorhagy. This loss of functional melanocytes may originate from multiple factors, including metabolic abnormalities, oxidative stress, generation of inflammatory mediators, autoimmune responses and cell detachment.

This basal detachment of melanocytes is associated with a disrupted surface distribution of adhesion molecule E-cadherin and the release of soluble E-cadherin. E-cadherin is an important adhesion glycoprotein responsible for maintaining the location of melanocytes within the basal layer of the skin epidermis. Disruption of E-cadherin adhesion leads to increases in both its soluble form and in suprabasal melanocytes, leading to dermal depigmentation.

E-cadherin cleavage may be induced by several proteases including MMP-9. MMP-9 is secreted by the keratinocytes in response to various proinflammatory cytokines such as IFN-7 and TNF-α. These type 1 cytokines are known to induce melanocyte detachment in both in vitro and in vivo models, and this effect is dependent on the inhibition of E-cadherin gene expression, internalization of E-cadherin, and its cleavage through the release of MMP-9 by keratinocytes.

In healthy skin, the stimulation of the WNT pathway by keratinocytes and melanocytes induces the differentiation and proliferation of melanocyte stem cells, allowing the constant turnover of the pools of epidermal melanocytes (see Regazzetti et al. Transcriptional Analysis of Vitiligo Skin Reveals the Alteration of WNT Pathway: A Promising Target for Repigmenting Vitiligo Patients, Journal of Investigative Dermatology, 135, 3105-3114 (2015)).

In vitiligo skin, oxidative stress can trigger the immune reaction in a genetically predisposed individual. The destruction of melanocytes by the immune system releases melanocyte antigens that stimulate an autoimmune response and ultimately lead to the complete disappearance of melanocyte from the epidermis (and sometimes the hair follicles). Concomitantly, oxidative stress decreases WNT pathway activity in melanocytes and in keratinocytes. This effectively induces decreased cell adhesion with detachment of melanocytes and an impaired differentiation of melanocyte stem cells, altering the capacity of melanocyte turnover. One hypothesis is depending on the patient and the course of the disease, these two mechanisms may be differentially implicated, leading to active depigmentation of the skin and resistance to repigmenting approaches (see e.g. Regazzetti et al. Journal of Investigative Dermatology 2015, 135, 3105-3114).

Moreover, compared with normal skin, vitiligo lesional (depigmented) skin typically contains genes (mostly melanocyte-specific genes) whose expression is decreased or absent. Unlike vitiligo lesional skin, most of the genes that are down-regulated in vitiligo lesional skin (including the melanocyte markers) show no such change in non-lesional (pigmented) skin (see Yu R, et al. Transcriptome analysis reveals markers of aberrantly activated innate immunity in vitiligo lesional and non-lesional skin. PLoS One. 2012; 7(12):e51040; see also Moretti et al., Focus on vitiligo: a generalized skin disorder, Eur J Inflamm., vol. 4, no. 1, 21-30, 2006).

The present disclosure includes the effects of BETi1 compositions at different concentrations (e.g., doses) and their vehicle on vitiligo, e.g., using a 3D skin model consisting of reconstructed human epidermis stimulated with a cocktail of cytokines (IFN-γ and TNF-α) to induce melanocytorhagy (RHE-VIT). The BETi1 compositions may be applied topically on the stratum corneum of the RHE-VIT, and compared to the vehicle composition and Ruxolitinib cream 1.5% (positive control) uninduced untreated (healthy skin) and induced untreated skin models.

The influence of these compositions on vitiligo model may be assessed, e.g., using one or more of the following methods: (i) Measurement of melanin content based on Fontana-Masson images of paraffin-embedded epidermis sections; (ii) Paraffin-embedded sections of epidermis to analyze the localization of the melanocytes throughout the epidermis by TRP-1 (Tyrosinase Related Protein-1) immunostaining; (iii) Cell culture media to quantify the release of the 2 biomarkers MMP-9 and E-Cadherin by specific ELISA assays.

The influence of the composition on the expression of 92 genes playing key roles in melanogenesis (melanin synthesis, melanosome maturation and transport) may be assessed by RT-qPCR (Reverse Transcription—quantitative Polymerase Chain Reaction) using TaqMan Low Density Array (TLDA) technology.

In some embodiments, to confirm an absence of cytotoxic effect of the compositions and vehicle on the 3D model, a preliminary morphological analysis may be conducted after staining, e.g., after hemalum/eosin staining, of sections of paraffin-embedded tissues.

In some embodiments, an immediate release composition, including larger suspended particles for a prolonged residency and release is provided, comprising at least one BETi disclosed herein (e.g., BETi1) used to treat or prevent a joint or joint related disorder or disease by local administration of the composition. In some embodiments, a sustained release composition is provided.

In some embodiments, the local administration of a composition comprising at least one BETi (e.g., BETi1) is via, for example, injection into at least one space chosen from intra-articular space, peri-articular space, soft tissues, lesions, epidural space, perineural space, or foramenal space at or near the site of a subject's joint pain or discomfort. In some embodiments, the composition further comprises at least one immediate release component. In some embodiments, a composition comprising at least one BETi (e.g., BETi1) is administered (e.g., by single injection or as sequential injections) into an intra-articular space for treatment of a joint or joint related disorder or disease, such as arthritis, bursitis, Ehlers-Danlos syndrome, epicondylitis, Felty Syndrome, gouty arthritis, psoriatic arthritis, osteoarthritis, rheumatoid arthritis, Still's disease, tenosynovitis, synovitis, Sjögren's Syndrome, lyme disease, Whipple disease, bone cancer, lupus, or other autoimmune joint disorder. In some embodiments the composition is formulated for immediate release. In some embodiments, the composition is formulated for immediate and prolonged release. The term “immediate release” is in one or more embodiments intended to include formulations providing immediate and prolonged release, achieved through appropriate particle size distribution. In some embodiments, the formulation disclosed herein in Method S may be suitable for monthly injections. In some embodiments, the presence/time in the joint may be improved by altering the particle size and distribution of the suspended API. It may also be further increased by preparing an extended-release formulation.

In some embodiments, the formulation disclosed herein is formulated for sustained release. In some embodiments it is formulated for both immediate and sustained release. In some embodiments, an immediate release composition comprising at least one BETi (e.g., BETi1) is administered. In some embodiments, a sustained release composition comprising at least one BETi (e.g., BETi1) is administered. In some embodiments, a combined initial and sustained release composition comprising at least one BETi (e.g., BETi1) is administered. In some embodiments, the sustained release formulation is a suspension of at least one BETi, i.e., one or two or more BETi, in a vehicle or carrier. In some embodiments, the suspension comprises two or more average particle sizes of the BETi compound or compounds (at least one larger and one smaller). In some embodiments, the smaller particles release at a higher rate than the larger particles. In some embodiments, at least a part of the particles are coated to facilitate a slower release rate. In some embodiments, the compound or compounds are encapsulated, e.g., in microparticles or microspheres.

In some embodiments, a sustained release composition comprising BETi1 is administered (e.g., by single injection or as sequential injections) into soft tissues or lesions for treatment of inflammatory disorders, such as rheumatoid arthritis.

In some embodiments, a composition for controlled or sustained release is provided and comprises a microparticle matrix (such as PLGA, gels, hydrogels, hyaluronic acid, etc.) and at least one BETi. The composition may or may not rapidly release the BETi for a first length of time of between 0 and 10 days, for example, between the beginning of day 1 through the end of day 10, in addition to the sustained, steady state release of the BETi for a second length of time of at least a week, at least two weeks, or at least three weeks, including up to and beyond one month, or two month, or three months or more.

In some embodiments, for immediate release and/or controlled release, the dose of BEti for intra-articularly administration in humans is about 1 mg to about 700 mg. In some other embodiments, the dose of BETi for intra-articularly administration in humans is about 1 mg to about 10 mg. For immediate release and/or controlled release, the volume of BETi1 dispersion injected into the joint is about 5 mL. Therefore, a dose of 500 mg BETi corresponds to 5 mL of a product having a concentration of 10%/w. The homogeneity (including absence of aggregates of solid particles) of the dispersion is analyzed and confirmed by light microscopy.

In some embodiments, the dose for an immediate release and/or immediate and prolonged release is about 10 mg to about 500 mg BETi, or about 10 mg to about 150 mg BETi, or about 10 mg to about 80 mg BETi. In some embodiments, the dose for controlled release is about 10 mg to about 250 mg BETi, or about 10 mg to about 120 mg BETi, or about 10 mg to about 70 mg BETi. In some embodiments, the dose for sustained or extended release is about 30%, about 35%, about 40%, or about 50% lower than that for controlled release. In some embodiments, the dose for immediate release is about twice the dose for sustained release.

In some embodiments, a composition for controlled or sustained release is provided comprising a microparticle matrix (such as PLGA, gels, hydrogels, a hyaluronic acid, etc.) and at least one BETi.

In some embodiments, the disclosed formulations comprising the BETi may provide an initial release of the BETi at the site of administration, for example, in the intra-articular space and/or peri-articular space. In some embodiments, after the initial release of BETi has subsided, the controlled or sustained release of the BETi microparticle formulations continues to provide therapeutic (e.g., intra-articular and/or peri-articular) concentrations of BETi to suppress inflammation, maintain analgesia, and/or slow, arrest or reverse structural damage to tissues for an additional period of therapy following administration.

In some embodiments there is provided an intra-articular composition comprising a BETi (e.g., BETi1). In some embodiments, the intra-articular composition is encapsulated in a PLGA microsphere. In some embodiments, the PLGA microsphere entraps the BETi (e.g., BETi1), and provides a slow-release thereof upon degradation of the PLGA within the joint. In some embodiments, the ratio of lactic acid to glycolic acid is about 50:50, about 65:35, about 70:30, about 75:25, or about 80:20 for the PLGA microsphere. In some embodiments, the ratio of lactic acid to glycolic acid is about 80:20 to about 60:40 or is about 80:20 to about 70:30. In some embodiments, the drug (i.e., BETi) load in the microspheres is between the range of about 10% to about 80%. In some embodiments, the drug load in the microspheres is about 25% w/w. In some embodiments, the composition comprises a hyaluronic acid. In some embodiments, the hyaluronic acid has high molecular weight, which can provide higher viscosity. In some embodiments, the hyaluronic acid has a medium level molecular weight. In some embodiments, the hyaluronic acid has a low molecular weight. The molecular weight of hyaluronic acid is variable and spans a wide range from a few thousand to several million kDa. In human normal synovial fluid, it is equal to about 6000-7000 kDa, while in rheumatoid fluid, the molecular weight is less, and is equal to about 3000-5000 kDa. In some embodiments, the hyaluronic acid is cross-linked to a polymer in order to increase viscosity and slow down degradation within the joint. In some embodiments, there is provided a method of restoring visco-elasticity to the synovial fluid. In some embodiments, the PLGA microsphere composition comprises a hyaluronic acid. In some embodiments, the PLGA microsphere composition comprising a hyaluronic acid provides or helps restore visco-elasticity to a joint e.g., to the synovial fluid. In some embodiments, the BETi (e.g., BETi1) is entrapped within hyaluronic acid. In other embodiments, the BETi (e.g., BETi1) is entrapped within the cross-linked hyaluronic acid polymer in order to provide a sustained release. In some embodiments, the PLGA microsphere composition comprises a lubricin. In some embodiments, the composition comprises a lubricin to provide or help restore lubrication to a joint. In some embodiments, the PLGA microsphere composition comprises a hyaluronic acid and a lubricin. In some embodiments, the composition comprises a hyaluronic acid and a lubricin to provide or help restore visco-elasticity and/or lubrication to a joint e.g., to the synovial fluid.

In some embodiments, there is an improvement in at least one symptom of a joint disorder or disease upon intra-arterial or intra-venous application of about 0.1% to about 10% of the BETi. In some embodiments, there is an improvement in at least one symptom of the joint disorder or disease upon local application within the joint of e.g., about 0.1% to about 15% of the BETi or about 0.2% to about 10% by weight of the composition or by weight to volume of the composition. In some embodiments the BETi is about 0.1%, or about 0.25%, or about 0.5%, or about 0.75%, or about 1%, or about 1.25%, or about 1.5% or about 1.75%, or about 2%, or about 2.25%, or about 2.5%, or about 2.75%, or about 3%, or about 3.25%, or about 3.5%, or about 3.75%, or about 4%, or about 4.25%, or about 4.5% or about 4.75%, or about 5%, or about 5.5%, or about 6%, or about 6.5%, or about 7%, or about 7.5%, or about 8%, or about 8.5%, or about 9%, or about 9.5%, or about 10%, or about 11%, or about 12%, or about 13%, or about 14%, or about 15% by weight of the composition or by weight to volume of the composition. In some embodiments application of the BETi is e.g., inter-articular and for immediate release and may be administered in a single dose of about 1 mg/vial to about 750 mg/vial, or about 2 mg/vial to about 500 mg/vial, wherein a vial is about 5 ml or about 2.5 ml or about 1 ml depending on the size of the joint and the age and size of the subject. In some embodiments the BETi for immediate release and may be administered in a single dose of about 1 mg/vial, or about 2 mg/vial, or about 3 mg/vial, or about 4 mg/vial, or about 5 mg/vial, or about 6 mg/vial, or about 7 mg/vial, or about 8 mg/vial, or about 9 mg/vial, or about 10 mg/vial, or about 25 mg/vial, or about 50 mg/vial, or about 75 mg/vial, or about 100 mg/vial, or about 150 mg/vial, or about 200 mg/vial, or about 250 mg/vial, or about 300 mg/vial, or about 350 mg/vial, or about 400 mg/vial, or about 450 mg/vial, or about 500 mg/vial, or about 550 mg/vial, or about 600 mg/vial, or about 650 mg/vial, or about 700 mg/vial, or about 750 mg/vial. In some embodiments application of the BETi is inter-articular and for sustained release and may be administered in a single dose of about 1 mg/vial to about 350 mg/vial, or about 2 mg/vial to about 250 mg/vial, wherein a vial is about 5 ml or about 2.5 ml or about 1 ml depending on the size of the joint and the age and size of the subject. In some embodiments the BETi is for sustained release and may be administered in a single dose of about 1 mg/vial, or about 2 mg/vial, or about 3 mg/vial, or about 4 mg/vial, or about 5 mg/vial, or about 6 mg/vial, or about 7 mg/vial, or about 8 mg/vial, or about 9 mg/vial, or about 10 mg/vial, or about 25 mg/vial, or about 50 mg/vial, or about 75 mg/vial, or about 100 mg/vial, or about 150 mg/vial, or about 200 mg/vial, or about 250 mg/vial. In some embodiments where the composition provides an immediate or initial dose and a sustained release dose it may be administered in a single dose of about 1 mg/vial to about 750 mg/vial, or about 2 mg/vial to about 500 mg/vial, or about 2 mg/vial to about 250 mg/vial, wherein a vial is about 5 ml or about 2.5 ml or about 1 ml depending on the size of the joint and the age and size of the subject.

A previously noted, unless specified otherwise, an amount expressed in terms of weight (whether absolute weight or relative to another weight or a volume or as a percentage) of a BETi or of “at least one compound, tautomer, stereoisomer, pharmaceutically acceptable salt, hydrate, and/or deuterated derivative” is based on the weight of the free base of the BETi or compound, respectively.

The present disclosure provides exemplary BETi that additionally can provide a new and effective treatment and relief for joint related diseases and disorders. Joints may be infected by many types of microorganisms (bacteria, fungi, viruses) and occasionally by animal parasites. Infection related joint diseases and disorders include infection by direct contamination, by way of the bloodstream e.g., through the synovial blood vessels, and by extension from adjacent bony infections (osteomyelitis). Infectious arthritis may affect one joint (monarthritis) or a few joints (oligoarthritis) rather than many (polyarthritis). Joints or parts thereof can be damaged e.g., cartilage by for example through staphylococci, hemolytic streptococci, and pneumococci infections, e.g., bone through tuberculosis such as tuberculous spondylitis (Pott disease), or through coccidioides immitis, brucellosis, such as brucella suis, leprosy (Hansen disease), rubella (German measles) and serum hepatitis, viral synovitis, dranunculiasis (Guinea worm disease), sexually transmitted diseases, including gonorrhea, reactive arthritis (Reiter disease), congenital syphilis such as Clutton joint lesion, and Yaws, which leads to skeletal lesions. Inflammation may destroy the joint cartilage and underlying bone and cause irreparable deformities. Adhesions between the articulating members are frequent in such cases, and the resulting fusion with loss of mobility is called ankylosis such as ankylosing spondylitis, (Marie-Strumpell disease or Bechterew disease). Another type of arthritis is associated with chronic intestinal diseases—ulcerative colitis, regional enteritis, inflammatory bowel disease, cirrhosis, and Whipple disease. In addition to joint disorders and diseases resulting from any of the above the present disclosure provides exemplary BETi that may also provide a new and effective treatment or relief for noninflammatory joint diseases, injury and degenerative disorders. Trauma to joints includes blunt injuries, mild sprains, fractures and dislocations, ligamentous, tendinous, and capsular tears, tears in the semilunar cartilages (menisci), and hemarthrosis. Degenerative joint disease includes osteoarthritis, arthrosis deformans, precocious osteoarthritis congenital dysplasia malum coxae senilis, spondylosis, chrondromalacia patellae, metabolic diseases such gouty arthritis, podagra, ochronotic arthropathy, chondrocalcinosis, or pseudogout, mucopolysaccharidoses, Hurler syndrome, Morquio disease, and polyepiphyseal dysplasias.

The present disclosure provides exemplary BETi that may also provide a new and effective treatment or relief for secondary joint diseases and disorders, including hemorrhagic joints, hemarthrosis, villonodular synovitis, joint diseases that arise in association with aseptic necrosis e.g., can occur with fractures, osteochondritis dissecans, slipped epiphysis, Osgood-Schlatter, Legg-Calvé-Perthes, endocrine-malfunctioning resultant joint disorders, acromegaly, neurogenic arthropathy, Charcot joint, hypertrophic osteoarthropathy, reflex sympathetic dystrophy, joint tumors, synovial chondromatosis, cartilaginous nodules, synovial osteochondromatosism, synoviomas, synovial sarcomas, and polymyalgia rheumatica.

In some embodiments, an immediate release form of a composition comprising a BETi (e.g., BETi1) is administered to treat a joint or joint related disorder or disease or a secondary joint disease or disorder e.g., by local application. In some embodiments the immediate release form comprises a solution of the BETi. In some embodiments the immediate release form comprises a suspension of the BETi as a micronized or nanoparticle suspension of the BETi. In some embodiments the immediate release form comprises the BETi partly in solution and partly suspended. In some embodiments the majority of the BETi is in solution (e.g., about 55%, about 60%, about 65%, about 70%, about 75% about 80%, about 85%, about 90%, about 95% or more). In some embodiments the majority of the BETi is suspended (e.g., about 55%, about 60%, about 65%, about 70%, about 75% about 80%, about 85%, about 90%, about 95% or more). In some embodiments, a sustained release form of a composition comprising a BETi (e.g., BETi1) is administered to treat a joint or joint related disorder or disease or a secondary joint disease or disorder e.g., by local application. In some embodiments the suspended particles provide for a sustained release following an initial release. In some embodiments the sustained release form comprises a particle or encapsulated suspension of the BETi. In some embodiments the sustained release form comprises the BETi partly suspended to provide for delayed release and partly in solution to provide for an initial release. As will be appreciated by one skilled in the art the proportion of suspended to dissolved will depend at least in part on the vehicle or carrier and the amount of the BETi.

In one or more embodiments, there is provided a BETi compound or composition comprising a BETi suitable for treating a fibrosis or fibrosis-related condition. The development of fibrotic conditions, whether induced or spontaneous, is caused at least in part by the stimulation of fibroblast activity. The influx of inflammatory cells and activated fibroblasts into the injured organ depends on the ability of these cell types to interact with the interstitial matrix, which contains primarily collagen. Exemplary tissues that may be affected by fibrosis include the lung, kidney, liver, skin, central nervous system, bone, bone marrow, tissues of the cardiovascular system, endocrine organs, and tissues of the gastrointestinal system.

In one or more embodiments, the fibrosis is in the lung. PF may result from a pathological wound healing process in which connective tissue replaces normal parenchymal tissue, leading to tissue remodelling, and the formation of permanent scar tissue. Fibrosis is similar to the process of scarring, in that both involve stimulated fibroblasts laying down connective tissue, including collagen and glycosaminoglycans.

The generation of granulation tissue is a carefully orchestrated process in which the expression of protease inhibitors and extracellular matrix proteins is upregulated, and the expression of proteases is reduced, leading to the accumulation of extracellular matrix. Abnormal accumulation of fibrous materials, however, may ultimately lead to organ failure.

The fibrotic process is initiated when immune cells such as macrophages release soluble factors that stimulate fibroblasts. The most well characterized pro-fibrotic mediator is Transforming Growth Factor-β(TGF-β), which is released by macrophages as well as any damaged tissue between surfaces called interstitium. Other soluble mediators of fibrosis include connective tissue growth factor (CTGF), platelet-derived growth factor (PDGF), and interleukin 10 (IL-10).

These initiate signal transduction pathways such as the AKT/mTOR and SMAD pathways that ultimately lead to the proliferation and activation of fibroblasts, which deposit extracellular matrix into the surrounding connective tissue. This process of tissue repair is a complex one, with tight regulation of extracellular matrix (ECM) synthesis and degradation ensuring maintenance of normal tissue architecture. However, the entire process can sometimes lead to a progressive irreversible fibrotic response if tissue injury is severe or repetitive, or if the wound healing response itself becomes deregulated.

In the lung myofibroblasts (a state between a fibroblast and a smooth muscle cell) are primarily involved in fibrosis. They are implicated in wound strengthening by extracellular collagen fiber deposition and then wound contraction by intracellular contraction and concomitant alignment of the collagen fibers by integrin-mediated pulling on to the collagen bundle. In wounds that fail to resolve and become keloids or hypertrophic scars, myofibroblasts may persist, rather than disappear by apoptosis. Myofibroblast formation is a TGF-β 1 dependent differentiation from fibroblast cells. Activation of the TGF-β receptor 1 and TGF-β receptor 2 leads to induction of the canonical SMAD2/SMAD3 pathway. Together with the co-activation of the non-canonical EGFR pathway, these events lead to upregulation of the ACTA2 gene and subsequent alpha smooth muscle actin protein production.

The methods and compositions of the present disclosure can in some embodiments be useful therapeutically for a fibrosis or fibrosis-associated conditions affecting any tissue including, for example, fibrosis of an internal organ, a cutaneous or dermal fibrosing disorder, and fibrotic conditions of the eye. In some embodiments, the fibrosis or fibrosis-associated conditions include fibrosis of internal organs (e.g., liver, lung, kidney, heart blood vessels, gastrointestinal tract). In some embodiments, the fibrosis or fibrosis-associated conditions include pulmonary fibrosis, idiopathic fibrosis, autoimmune fibrosis, myelofibrosis, liver cirrhosis, veno-occlusive disease, mesangial proliferative glomerulonephritis, crescentic glomerulonephritis, diabetic nephropathy, renal interstitial fibrosis, renal fibrosis in subjects receiving cyclosporin, allograft rejection, HIV associated nephropathy. In some embodiments, the fibrosis-associated disorders include systemic sclerosis, eosinophilia-myalgia syndrome, and fibrosis-associated CNS disorders such as intraocular fibrosis. In some embodiments, dermal fibrosis disorders include, for example, scleroderma, morphea, keloids, hypertrophic scars, familial cutaneous collagenoma, and connective tissue nevi of the collagen type. In some embodiments, fibrotic conditions of the eye include conditions such as diabetic retinopathy, post-surgical scarring (for example, after glaucoma filtering surgery and after crossed-eyes (strabismus) surgery), and proliferative vitreoretinopathy. In some embodiments, fibrotic conditions that may be treated by the methods of the present invention may result, for example, from rheumatoid arthritis, diseases associated with prolonged joint pain and deteriorated joints, progressive systemic sclerosis, polymyositis, dermatomyositis, eosinophilic fascitis, morphea, Raynaud's syndrome, and nasal polyposis.

The present disclosure includes the effects of BETi1 compositions at different concentrations (e.g., doses) and their vehicle on PF, e.g., using a bleomycin-induced mouse model of Idiopathic P F. The BETi1 compositions may be applied topically (e.g., by inhalation) in bleomycin-induced mice, and may be compared to bleomycin-induced mice treated with the vehicle composition or uninduced mice (healthy mice) treated with vehicle.

The influence of these compositions on an IPF mouse model may be assessed, e.g., using one or more of the following methods: (i) hydroxyproline content of lung tissue measured by colorimetric assay; (ii) fixed lung tissue stained with H & E, MT and/or PSR staining; (iii) clinical observation (body weight, weight of whole lung and probability of survival); and (iv) histogram of the oxygen saturation as measured by Pulse oximetry.

In some embodiments, to confirm an absence of cytotoxic effect of the compositions and vehicle in mice, a preliminary morphological analysis may be conducted after staining, e.g., after hemalum/eosin staining, of sections of lung—embedded tissues.

A non-exhaustive list of methods of administration of the compounds includes, topically on a body surface, such as skin, an epithelial surface, or a mucosa. The body surface can be external, such as the skin or eye, or it can be an internal body cavity surface, such as the mucosa within the respiratory tract, respiratory system, lung, intestinal tract, or vagina. Application can be through the airway passages, (e.g., by inhalation spray, by inhalation nebulizer, by inhaler (dry powder or metered), and by insufflation). Application may be, in some embodiments intramucosally, transmucosally, intrapulmonary, intradermal, nasally, buccally, rectally, transdermally, sublingually, and vaginally.

Unless defined otherwise, all technical and scientific terms used herein have the same meaning as is commonly understood by one of ordinary skill in the art to which this disclosure belongs. All ranges disclosed herein include the endpoints. The use of the term “or” shall be construed to mean “and/or” unless the specific context indicates otherwise. All patents, applications, published applications, and other publications are incorporated by reference in their entirety. In the event that there is a plurality of definitions for a term herein, those in this section prevail unless stated otherwise.

All % values are provided on a weight (w/w) basis unless otherwise indicated.

Various carriers and compositions or formulations are described herein. They are often described for use in a method. A reference to or example of a carrier, composition or formulation for use in one method does not in any way limit the carrier, composition or formulation for use just in that method, but it can be for use in any other method or embodiment described herein. The carriers, compositions or formulations described herein are in some embodiments provided as carriers, compositions or formulations and are in some embodiments provided as a product even where they are described only in relation to their use in a method.

As used herein, the term “about” has its usual meaning in the context of pharmaceutical and cosmetic formulations and methods of using the same to allow for reasonable variations in amounts that can achieve the same effect, typically plus or minus up to 30%. For example, if an amount of “about 1” is provided, then the amount can be up to 1.3 or from 0.70. In cases where “about X” will lead to a figure of above 100%, the term in one or more embodiments can be read as reflecting up to 100% by weight less the total of the minimum amount of the other ingredients. Likewise, it will be appreciated by one skilled in the art, to the extent X is reduced from that upper level, the amounts of the other ingredients are increased appropriately. As will be appreciated by one of skill in the art, there is some reasonable flexibility in formulating compositions such that where one or more ingredients are varied, successful formulations can still be made even if an amount falls slightly outside the range. Therefore, to allow for this possibility, amounts are qualified by about. In one or more embodiments, the examples e.g., amounts of formulation ingredients can be read as if prefixed with the term “about.” In one or more embodiments, the examples can be read without the term “about.” In one or more embodiments, the figures can be read with the term “about.” In one or more other embodiments, the figures can be read without the term “about.” In some narrower embodiments, “about” can be plus or minus up to 15% unless the context indicates otherwise. Where “about” is used in connection with “>X” or “<X”, or a series of such alternatives, it can, in one or more embodiments, include about X. Where “about” is used just at the beginning of a series of alternative amounts of “>about X” or “<about X” or “about >X” or “about <X”, it can, in one or more embodiments, be understood to include “about” before all the other alternatives of the series.

As used herein, the terms “composition(s)” and “formulation(s)” can be used interchangeably depending on the context in which they are used as would be appreciated by a person skilled in the art.

As used herein, the terms “mean” and “average” can also be used interchangeably depending on the context in which they are used as would be appreciated by a person skilled in the art.

The term “comprising” or variants thereof will be understood to imply the inclusion of a stated element, integer or step, or group of elements, integers or steps, but not the exclusion of any other element, integer or step, or group of elements, integers or steps.

The term “consisting” or variants thereof will be understood to imply the inclusion of a stated element, integer or step, or group of elements, integers or steps, and the exclusion of any other element, integer or step or group of elements, integers or steps.

As used herein, the term “selected from the group consisting of” can be understood to refer to a closed group. If the terms “chosen from” or “selected from” or “includes” or “comprises” are used, they encompass an open group.

As used herein, the terms “disorder(s)” and “disease(s)” can be used interchangeably depending on the context in which they are used as would be appreciated by a person skilled in the art.

The term “room temperature” or “RT” as used herein, means 20° C. to 25° C. In an embodiment it is 20° C. In an embodiment it is 21° C. In an embodiment it is 22° C. In an embodiment it is 23° C. In an embodiment it is 24° C. In an embodiment it is 25° C. The term “ambient conditions” as used herein means room temperature, pressure and humidity. Ambient temperature and room temperature are used interchangeably herein.

The term “thixotropic,” as used herein, means that the formulation shows a decrease in viscosity upon application of a shear force. The structure of the formulation breaks down, leading to a reduction in viscosity. When the formulation is left standing without shear force, the viscosity is recovered.

As used herein, the term “gel”, refers, inter alia, to a carrier or formulation or composition that is not flowable at room temperature, such that, when subjected to normal gravity at room temperature, it will retain its form. The term “flowable semi-solid”, as used herein refers, inter alia, to a base carrier or formulation that is slowly flowable when subjected to normal gravity at room temperature, and over time can adapt to and adopt the shape of a container. The term “liquid”, refers, inter alia, to a base carrier or formulation at room temperature, which is easily or readily flowable and can be poured into a container and can adapt to and adopt the shape of a container practically immediately.

As used herein, “foam” has its ordinary meaning to one of skill in the art, e.g., it may refer to an object or substance formed by trapping gas pockets within a solid or liquid. The gas pockets may comprise a gas, e.g., oxygen, nitrogen, or a mixture of gases, e.g., helium and xenon, or atmospheric air. The gas pockets within the foam may be connected to each other, e.g., closed-cell foams or discrete, e.g., open-cell foams. As used herein, “foamable compositions” refers to any composition that can form a foam. In some embodiments, foamable compositions comprise a carrier with or without a liquefied or compressed gas propellant, that forms a foam when the carrier is brought in contact with the propellant or by mechanical means, such as an air pump. In some embodiments, a foamable composition is packaged in an aerosol container together with a pressurized propellant. In some embodiments, the foamable composition is separate from the propellant such as in a bag in can system. In some embodiments, a valve on the aerosol container is actuated to release the foamable composition to form a foam. In some embodiments the composition is not a foam or does not form a foam on release or in use.

As used herein, the terms “water based” or “aqueous” or “hydrous” formulations refer to compositions that contain water as the majority component. In some embodiments, a formulation disclosed herein comprises water. In some embodiments, a formulation disclosed herein is based on water as the majority component. In some embodiments, the formulation comprises about >50%, about >55%, about >60%, about >65%, about >70%, or about >75% about >80%, about >85%, about >90%, or about >95% water by weight. In some embodiments, a formulation disclosed herein is water-free. As used herein, the terms “waterless” or “water-free,” or “anhydrous” or “nonaqueous” refer to compositions that contain no free or unassociated or absorbed water. In some embodiments, a waterless or water-free or anhydrous or nonaqueous composition comprises 0.0% added water by weight. Such a composition may contain trapped, bound, associated or otherwise unfree water, e.g., within its higher order crystal structure and, in some embodiments, such water may be about 1% or less. In some embodiments, the formulation is hydrophilic. In some embodiments, a formulation disclosed herein is water-free. The terms “essentially waterless” or “essentially water-free” or “essentially anhydrous” or “essentially nonaqueous” refer to compositions that comprise less than 0.05% of water by weight. In some embodiments, an essentially water-free or anhydrous or nonaqueous composition comprises 0.04%, 0.03%, 0.02%, or 0.01% water by weight. The terms “substantially water-free” or “substantially waterless” or “substantially anhydrous” or “substantially nonaqueous” refer to compositions that comprise less than 0.5% of water by weight. In some embodiments, a substantially water-free or anhydrous or nonaqueous composition comprises 0.4%, 0.3%, 0.2%, or 0.1% water by weight. As used herein, “low water” refers to a composition that contains about or less than 1% of water by weight. In some embodiments, a composition with low water comprises 0.9%, 0.8%, 0.7%, 0.6% or 0.5% of water by weight. In some embodiments, the composition is a substantially low water composition comprising about 5%, about 4%, about 3%, about 2%, or about 1% water by weight.

The term “single phase” as used herein means that, after preparation, the liquid components of the composition or carrier are fully miscible, and the solid components, if any, are either dissolved or homogeneously suspended in the composition so that only one phase is visible. In the context of a foamable composition “single phase” means that, after addition of propellant to the composition or carrier, the liquid components of the foamable composition or carrier are fully miscible, and the solid components, if any, are either dissolved or homogeneously suspended in the composition so that only one phase is visible. In some embodiments, a composition has a single phase before the addition of propellant. In some embodiments, a composition has a single phase after the addition of propellant.

By the term “substantially a single phase” it is meant that the composition or carrier, after preparation, is primarily or essentially a single phase, as explained above, but can also have present a small amount of material which is capable of forming a separate phase amounting to less than about 5% by weight of the composition or carrier, e.g., less than about 3% by weight, or less than about 1% by weight of the composition. In the context of a foamable composition by the term “substantially a single phase” it is meant that the composition or carrier, after addition of propellant, is primarily or essentially a single phase as explained above, but can also have present a small amount of material which is capable of forming a separate phase amounting to less than about 5% by weight of the composition or carrier after the addition of propellant, e.g., less than about 3% by weight, or less than about 1% by weight of the composition. In some embodiments a composition may be a single phase before addition of propellant and a single phase after addition of propellant. In some embodiments a composition may be substantially a single phase before addition of propellant and a substantially single phase after addition of propellant. In some embodiments a composition may be substantially a single phase before addition of propellant and a single phase after addition of propellant. In some embodiments a composition may be a single phase before addition of propellant and substantially a single phase after addition propellant. In some embodiments a composition may be a single phase before addition of propellant and substantially a single phase after addition of propellant.

Where embodiments of the present invention are discussed herein in terms of a method of treatment involving the administration of a formulation or composition, it will be understood that the invention also provides that formulation, composition or active ingredient(s) thereof for use in that method, as well as the use of the formulation, composition or active ingredient(s) thereof in the manufacture of a medicament for use in that method.

As used herein, the term “emollient” refers to a material or agent that, when placed in contact with the human skin, is able to soften, smoothen, reduce scaling and itching, reduce inflammation, improve skin barrier function, and/or act as a carrier for active agents. Examples of emollients include but are not limited to avocado oil, isopropyl myristate, mineral oil, capric triglycerides, caprylic triglyceride, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohols, cetyl acetate, phenyl trimethicone, glyceryl oleate, tocopheryl linoleate, wheat germ glycerides, arachidyl propionate, myristyl lactate, decyl oleate, ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, unsaturated or polyunsaturated oils, olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oil, an essential oil, a silicone oil, dimethicone, cyclomethicone, polyalkyl siloxane, polyaryl siloxane, polyalkylaryl siloxane, a polyether siloxane copolymer, and poly(dimethylsiloxane)-(diphenyl-siloxane). In some embodiments, the formulation comprises one or more emollients. In some embodiments, the emollient is hydrophilic or non-hydrophobic. In some embodiments, emollients are chosen that are compatible with use in joints including being able to be degraded so accumulation in the joint does not become an issue. In some embodiments, the formulation is free of oily or hydrophobic emollients. In some embodiments, a formulation disclosed herein is essentially free of oily or hydrophobic emollients. In some embodiments a formulation disclosed herein is substantially free of oily or hydrophobic emollients. The term “essentially free” or refer to compositions that comprise less than 0.05% (e.g., about <0.04%, about <0.03%, about <0.02%, or about <0.01%) of oily or hydrophobic emollients by weight. The term “substantially free” or refers to compositions that comprise less than 0.5% (e.g., about <0.4%, about <0.3%, about <0.2%, or about <0.1%) of oily or hydrophobic emollients by weight.

“Standard surfactant,” “customary surfactant” or “stand-alone surfactant” refer to customary non-ionic, anionic, cationic, zwitterionic, amphoteric and amphiphilic surfactants. Many standard surfactants are derivatives of fatty alcohols or fatty acids, such as ethers or esters formed from such fatty alcohols or fatty acids with hydrophilic moieties, such as polyethylene glycol (PEG). However, a native (non-derivatized) fatty alcohol or fatty acid, as well as waxes are not regarded as a standard surfactant.

The term “co-surfactant” as used herein means a molecule which, on its own, is not able to form and stabilize satisfactorily an oil-in-water emulsion, but, when used in combination with a surfactant as defined herein, the co-surfactant has properties which can allow it to help a surfactant create an emulsion and can boost the stabilizing power or effect of the surfactant. Examples of co-surfactants include fatty alcohols, such as cetyl alcohol, or fatty acids, such as stearic acid. Cetyl alcohol is a waxy hydrophobic substance that can be emulsified with water using a surfactant. Some substances can have more than one function and, for example, fatty alcohols can, in some formulations, act as a co-solvent. In certain circumstances, a co-surfactant can itself be converted into a surfactant or soap by, for example, adding a base, such as triethanolamine to a fatty acid like stearic acid. The term “modifying agent” as used herein is an agent which, when added to a hydrophobic oil, facilitates the creation of a hydrophobic breakable vehicle in the form of a gel. In some embodiments, it can facilitate the formation of a breakable gel or breakable foam, a thixotropic gel, or an elastic gel.

As used herein, a “foamer complex,” a “foam stabilizer” or a “foam adjuvant”, in relation to a foamable composition can comprise, e.g., a fatty alcohol, a fatty acid and/or a wax. In some embodiments, the foam adjuvant is a fatty alcohol and a wax or a fatty acid and a wax. In some embodiments, it is a wax. In some embodiments, the foam adjuvant or modifying agent comprises at least one of a fatty alcohol, a wax, or a fatty acid. In some embodiments, the foam adjuvant or the modifying agent is selected from a group consisting of a fatty alcohol, a wax, and a fatty acid. In some embodiments, the foam adjuvant is a fatty alcohol. In some embodiments, the foam adjuvant is a fatty acid. In some embodiments, the foam adjuvant is a wax. In some embodiments, a wax has the properties of a foam adjuvant. In some embodiments, a fatty alcohol, and/or a fatty acid, and/or a wax is an adjuvant. In some embodiments, the composition is free, or is essentially free or is substantially free of a foam adjuvant.

As used herein, a formulation disclosed herein may additionally include one or a combination of waxes.

The term “breakable” refers to a property of a gel wherein the gel is stable upon dispensing from a container yet breaks and spreads easily upon application of shear or mechanical force, which can be mild, such as a simple mechanical rub.

The term “water activity”, as used herein, represents the hygroscopic nature of a substance, or the tendency of a substance to absorb water from its surroundings. Microorganisms require water to grow and reproduce, and such water requirements are best defined in terms of water activity of the substrate. The water activity of a solution is expressed as Aw=P/Po, where P is the water vapor pressure of the solution and Po is the vapor pressure of pure water at the same temperature. Every microorganism has a limiting Aw, below which it will not grow, e.g., for Streptococci, Klebsiella spp, Escherichia coli, Clostridium perfringens, and Pseudomonas spp, the Aw value is 0.95. Staphylococcus aureus is most resistant and can proliferate with an Aw as low as 0.86, and fungi can survive at an Aw of at least 0.7.

The identification of a “solvent”, as used herein, is not intended to characterize the solubilization capabilities of the solvent for any specific active agent or any other component of the composition or foamable composition. Rather, such information is provided to aid in the identification of materials suitable for use as a component of the composition or foamable composition described herein.

As used herein, the term a “hydrophilic solvent” refers to a solvent that has a solubility in distilled water at ambient temperature of more than about 1 gm per 100 mL, or more than about 0.5 gm per 100 mL, or even more than about 0.1 gm per 100 mL. The hydrophilic solvent, in one or more embodiments, remains a liquid at ambient temperature.

The terms “hydrophilic gel composition” or “hydrophilic flowable semi-solid composition” or “hydrophilic liquid composition” or “hydrophilic composition” or “hydrogel” or “hydrogel composition”, as used herein, refer to or include compositions that are comprised mainly of ingredients, which are soluble in water or miscible/mixable with water and may be aqueous or non-aqueous or, in either case, essentially or substantially so. The ingredients can include inorganic or organic excipients, of various molecular weights, and may include polymers. As used herein, the term “gel”, refers, inter alia, to a carrier or formulation or composition that is not flowable at room temperature, such that when subjected to normal gravity at room temperature, it will retain its form. The term “flowable semi-solid”, as used herein refers, inter alia, to a base carrier or formulation that is slowly flowable when subjected to normal gravity at room temperature, and, over time, can adapt to and adopt the shape of a container. The term “liquid”, refers, inter alia, to a base carrier or formulation at room temperature, which is easily or readily flowable and can be poured into a container and can adapt to and adopt the shape of a container practically or almost immediately.

The terms “hydrophobic gel composition” or “hydrophobic flowable semi-solid composition” or “hydrophobic liquid composition” or “hydrophobic foamable composition” or “hydrophobic foam composition” or “hydrophobic aerosol composition” or “hydrophobic composition”, as used herein, refer to compositions that have a low solubility in water. In some embodiments, 100 to 1000 parts of water are needed to dissolve or render miscible 1 part of the composition. In some embodiments, 1000 to 10,000 parts of water are needed to dissolve or render miscible 1 part of the composition. In some embodiments, more than 10,000 parts of water are needed to dissolve or render miscible 1 part of the composition. In some embodiments, the formulation is not a hydrophobic composition, or hydrophobic liquid composition, or hydrophobic flowable semi-solid composition, or hydrophobic gel composition.

It should be noted that the term “substantially free of” an ingredient, as used herein, is intended to mean that the composition comprises less than about 0.5% by weight of the ingredient, unless specifically indicated otherwise.

As used herein, the term “essentially free of” an ingredient, as used herein, is intended to mean that the composition comprises less than about 0.05% by weight of the ingredient, unless specifically indicated otherwise.

By “essentially free of a steroid” means an amount of steroid that is not a therapeutically effective amount or an amount of less than about 0.05% by weight, less than about 0.04% by weight, than about 0.03% by weight, less than about 0.02% by weight, less than about 0.01% by weight, less than about 0.008% by weight, less than about 0.006% by weight, than about 0.004% by weight, less than about 0.002% by weight, or less than about 0.001% by weight.

In some embodiments, the composition is essentially free of or free of a steroid. In some embodiments, the composition is essentially free of or free of a betamethasone, e.g., betamethasone valerate. In some embodiments, the composition is essentially free of or free of a triamcinolone, e.g., triamcinolone acetonide. In some embodiments, the composition is essentially free of or free of a dexamethasone, e.g., dexamethasone sodium phosphate. In some embodiments, the composition is essentially free of or free of a clobetasol, e.g., clobetasol propionate.

As used herein, the term “free of” an ingredient used herein, is intended to mean that the composition does not comprise any amount of the ingredient, unless specifically indicated otherwise, e.g., where the ingredient is present in a trapped, bound, associated or otherwise unfree state. In some embodiments, an ingredient will be considered as containing constituents normally found present in a trapped, bound, associated or otherwise unfree state, all in accordance with the grade of purity of the ingredient.

The terms “surfactant-free” or “emulsifier-free” or “non-surfactant” refer to compositions that comprise no or negligible levels of surfactants, emulsifiers, or surface-active agents. Where a formulation includes insignificant or de minimis amounts of surfactants, emulsifiers, or surface-active agents, it is considered to be essentially surfactant-free. As used herein, “essentially free of surfactant” indicates less than about 0.05% by weight of a surfactant, e.g., a surfactant chosen from or is selected from the group consisting of non-ionic, ionic, anionic, cationic, zwitterionic, amphoteric and ampholytic surfactants. The term “substantially surfactant-free” relates to a composition that contains a total of about or less than 0.5% by weight of surfactant, e.g., a surfactant chosen from or is selected from the group consisting of non-ionic, ionic, anionic, cationic, zwitterionic, amphoteric, and ampholytic surfactants. In some embodiments, the composition comprises about or less than 0.2% by weight of a surfactant; about or less than 0.15% by weight; about or less than 0.1% by weight; about or less than 0.05% by weight; or about or less than 0.01% by weight.

As used herein, the term “preventing” refers to avoiding the onset of a disorder or condition from occurring in a subject that has not yet been diagnosed as having the disorder or condition, but who may be susceptible to it.

The term “polyol” as used herein is an organic substance that contains at least two hydroxy groups in its molecular structure.

As used herein, the term “treatment” or “treating” refers to inhibiting, ameliorating, or reducing the disorder or condition, e.g., arresting its development; reversing or relieving the disorder or condition, e.g., causing regression of the disorder or condition or reversing the progression of the disorder or condition; slowing progression of or relieving or reducing, arresting, or reversing one or more symptoms of the disorder or condition (e.g., damage associated with a joint disorder or disease). In some embodiments, “treatment” or “treating” means preventing or helping to prevent the disorder or condition or one or more symptoms thereof. In other words, the term “treatment” defines the therapeutic treatment of a human or non-human animal, to impede or reduce or halt the rate of the progress of the condition, or to ameliorate or cure the condition. In some embodiments, “treatment” or “treating” includes slowing, arresting, or reversing damage associated with a disorder or disease. Prophylaxis of the condition as a result of treatment is also included. References to prophylaxis are intended herein not to require complete prevention of a condition: its development may instead be hindered through treatment in accordance with the invention. Typically, treatment is not prophylactic, and the compound or composition is administered to a patient having a diagnosed or suspected condition. By an “effective amount” herein defines an amount of the compound or composition that is sufficient to impede the noted diseases and thus to produce the desired therapeutic or inhibitory effect.

The phrase “a method of treating or preventing a disease or a disorder,” such as a joint or joint related disorder or disease, or a fibrosis or respiratory related disorder or disease, as provided throughout the specification is interchangeable with “use of a composition as a medicament for treatment or prevention (or prophylaxis) of a disease or a disorder”. The terms “method(s) of” e.g., “preventing”, “prophylaxis”, or “treating” a disease or a disorder provided throughout the specification is interchangeable with the terms “use of” or “for use. It should be noted that the term “disease” is used interchangeably with the term “disorder.”

The term, “Psorasis Area and Severity Index Score”, also known as “PASI,” can be used to monitor the improvements in the skin component of psoriasis in a subject. The method for determining the PASI has been described in Fredriksson and Pettersson (1978) Dermatologica 157:238 and Marks et al. (1989) Arch Dermatol 125:235. Briefly, the index is based on evaluation of four anatomic sites, including the head, upper extremities, trunk, and lower extremities, for erythema, induration, and desquamation using a 5-point scale (0=no symptoms; 1=slight; 2=moderate; 3=marked; 4=very marked). Based on the extent of lesions in a given anatomic site, the area affected is assigned a numerical value (0=0; 1=<10%; 2=10-29%; 3=30-49%; 4=50-69%; 5=70=89%; 6=90-100%). The PASI score is then calculated, wherein the possible range of PASI score is 0.0 to 72.0 with the highest score representing complete erythroderma of the severest degree.

The term, “modified PASI,” also known as “MPASI,” ranges from 0 to 60 on a scale representing the proportion of area involved and the severity of erythema and desquamation, ranking from 0 (normal) to 4 (severe). The final sum gave the total score for erythema, induration and peeling. The modified PASI (mPASI) also referred herein as Composite Inflammation Severity Score is calculated in the same the manner as mean PASI score but excludes the scoring for induration (thickening of the skin). It is a composite mean score of erythema and peeling severity scored on a 4-point ordinal scale per domain (0=none, 1=mild, 2=moderate and 3=severe) for a maximum score of 6.

As used herein, the expression “pigmentation disorder and/or disease” or “pigmentation related disorder and/or disease” refers to a health condition that affects the color of the skin (e.g., either loss or reduction in color, e.g., typically due to loss or destabilization of melanocytes, or an increase in color, typically characteristic of over-pigmentation disorders, such as melasma and age spots. In one or more embodiments, the BET inhibitors are used on disorders where there is a loss or reduction in color. In some embodiments it is the loss or reduction of skin color. In some embodiments it is the loss or reduction of hair color. In some embodiments it is the loss or reduction of eye color. For example, vitiligo is due to the loss or destruction of melanocytes, which are the cells that produce melanin. Melanin determines the color of skin, hair, and eyes. If melanocytes cannot form melanin or if their number decreases, skin color becomes progressively lighter.

The term “cytokine” is a type of small protein (typically ˜5 to 20 kDa) involved in cell signaling, such as autocrine, paracrine and endocrine signaling, and/or as immunomodulating agents. Cytokines are produced by certain immune and non-immune cells and have an effect on the immune system. For example, some cytokines stimulate the immune system and others slow it down. Exemplary cytokines include interleukins and the like.

As used herein, “biocompatible biodegradable material” refers to materials that are biocompatible and biodegradable. The term “biocompatible” material refers to a material that is not toxic to the human body, is not carcinogenic, and it should induce limited or no inflammation in body tissues. A “biodegradable” material refers to a material that is degraded by bodily processes (e.g., enzymatic) to products readily disposable by the body or absorbed into body tissue. The biodegraded products should also be biocompatible with the body. In the context of intra-articular or respiratory drug delivery systems for the compounds disclosed herein, biocompatible biodegradable materials may be used to fabricate, for example, microparticles, micro-spheres, matrices, microparticle matrices, micro-sphere matrices, gels, hydrogels, rods, wafers, liposomes, fibers, pellets, or other appropriate pharmaceutical delivery compositions that a health care professional can administer into the joint or alternatively can administer systemically to reach the joint (e.g., capsules, and pills). The biocompatible biodegradable material may degrade into non-toxic residues that may be easily removed, broken down or dissolved by the body, and/or cleared from the body intact.

“Fibrosis” is defined as any pathological condition resulting from an overproduction or aberrant production of fibrous tissue. Fibrosis may occur in any organ including, for example, lung, kidney, liver, skin, central nervous system, bone, bone marrow, cardiovascular system, an endocrine organ or the gastrointestinal system. By “fibrosis-associated condition” is meant any condition that is related to fibrosis. Thus, fibrosis-associated conditions may be caused by, be concomitant with, or cause fibrosis.

By “treating or ameliorating fibrosis” is meant decreasing the level of fibrosis relative to an untreated control, as measured by any standard method. A reduction in fibrosis may also be measured by a reduction in any symptom associated with fibrosis or a fibrosis-associated condition. The examples disclosed herein provide exemplary methods of determining whether the level of fibrosis is decreased relative to a control.

By “treating or ameliorating a fibrosis-associated condition (or disorder)” is meant decreasing such condition before or after it has occurred. As compared with an equivalent untreated control or vehicle, such reduction or degree of prevention is at least about 5%, about 10%, about 20%, about 40%, about 50%, about 60%, about 80%, about 90%, about 95%, or about 100%. A subject who is being treated for a fibrosis-associated condition is one who a medical practitioner has diagnosed as having such a condition. A subject in whom the development of a fibrosis-associated condition is being prevented may or may not have received such a diagnosis. One in the art will understand that these subjects (e.g., patients) may have been subjected to standard tests for diagnosing fibrosis-associated conditions or may have been identified, without examination, as one at high risk due to the presence of one or more risk factors. Disclosed herein are exemplary methods of determining whether the level of a fibrosis-associated disorder is decreased relative to a control.

By “de minimis” it is meant to be so minor that its effect is to be disregarded, e.g., having no functional impact on a formulation or method.

As used herein, “chemically stable” refers to a compound (active agent or excipient) or a composition where no significant decrease in assay and no significant increase in impurities and no significant appearance of breakdown products may be observed at the conditions and during the time period tested. A decrease in assay or increase in impurities may occur for example, when a compound or a composition is oxidized, degraded, and/or reacts upon exposure to air, light, skin, mucosa, tissue, water, any pharmaceutical excipient, or any active agent under ambient conditions. By a significant decrease in assay is intended about or more than about 2% decrease of initial assay value. By a substantially significant decrease in assay is intended about or more than about 5% decrease of initial assay. By a significant increase in impurities or in breakdown products is intended about or more than about 2% decrease of the initial assay value. By a substantially significant increase in impurities or breakdown products is intended about or more than about 5% of the initial assay value. By no significant decrease in assay, it is intended less than 1% decrease of initial assay value. By no significant increase in related compounds or breakdown impurities, it is intended less than 1% increase of initial assay value. For clarity, if the initial assay value was 100% and the new assay value is 96% the decrease is 4%. Similarly, if the initial assay value for impurities or breakdown products is 0.1% and the new assay value is 1.1% the increase is 1%. In some embodiments, a significant or substantially significant decrease in assay and/or a significant or substantially significant increase in breakdown products/impurities, as described above, may occur in less than 24 hours, which could be e.g., less than 16 hours, less than 12 hours, less than 6 hours, less than 5 hours, less than 4 hours, less than 3 hours, less than 2 hours, or less than 1 hour, upon exposure under room temperature ambient conditions to, for example, air, light, respiratory system, skin, water, or pharmaceutical excipients or any active agent. The term “chemically unstable” refers to a compound or a composition that falls outside the above definition of chemically stable.

As used herein, the term “physically stable” refers to a compound or a composition where no significant change in its state is observed during the time period and conditions under which it is tested. For example, a physically stable formulation will allow for 5% or fewer agglomerates.

As used herein, the term “physically unstable” refers to a compound or a composition where a significant change in its state is observed during the time period and conditions under which it is tested. For example, a physically unstable formulation is one that results in apparent phase separation over time or conditions suitable for its use.

As used herein “homogenous” or “homogenous distribution” refers to the property of a composition, in which the particles and/or active agent and/or excipients are proportionally distributed throughout. In some embodiments, the compositions comprising a BETi compound are homogenous.

As used herein “micronized” refers to a substance reduced in size to a fine powder, the particles or crystals of which are measured in micrometers in diameter. A measurement of the particle or crystal size in suspension in a composition can be expressed as D90. If, for example, 90% of the particles or crystals in the suspension are less than 15 microns, then the D90 is 15 microns. In one or more embodiments, the D90 of the particles is less than about 50 μm. In some embodiments, it is less than 45 μm or less than 40 μm, or less than 35 μm, or less than 30 μm, or less than 25 μm, or less than 20 μm or less than 15 μm, or less than 10 μm. In some embodiments, the D90 is between 50 μm and 15 μm, 30 μm and 20 μm, or is between 25 μm and 15 μm, or is between 20 μm and 10 μm, or is between 15 μm and 5 μm, or is between 10 μm and 3 μm. A measurement of the particle or crystal size in suspension in a composition can alternatively be expressed as D50. If, for example, 50% of the particles or crystals in the suspension are less than 25 microns, then the D50 is 25 microns. In one or more embodiments, it is less than 25 μm or less than 20 μm, or less than 15 μm, or less than 10 μm, or less than 5 μm, or less than 2 μm or less than 1.5 μm, or less than 1 μm. In one or more embodiments, the D50 is between 25 μm and 5 μm, 20 μm and 10 μm, or is between 15 μm and 10 μm, or is between 10 μm and 3 μm, or is between 15 μm and 5 μm, is between 5 μm and 1 μm is between 3 μm and 0.1p m.

As used herein “non occlusive” refers to topical formulation or substance, which allows for significant trans-epidermal water loss (TEWL) initially when applied topically or locally as an unbroken layer on healthy skin. By significant is intended in some embodiments of more than 30% water loss after 20 minutes following application.

As used herein “TEWL” refers to the amount of water that passively evaporates through skin to the external environment due to water vapor pressure gradient on both sides of the skin barrier and is used to characterize skin barrier function. The average TEWL in human is about 300-400 mL/day; however, it can be affected by environmental and intrinsic factors. In high humidity, the amount of water loss will decrease due to the drop in the water vapor pressure gradient. TEWL varies in different anatomic sites and is inversely related to the corneocyte size. Skin sites with smaller corneocytes have higher TEWL values. Multiple instruments are commercially available to measure TEWL, providing valuable data with applications in clinical settings, toxicology, and product development. TEWL is a sensitive indicator of skin irritation and is widely used in objective analysis of irritancy potential or protective properties of topical products. To the extent TEWL is impacted by joint inflammation in the region of the inflammation, in one or more embodiments, application of a BETi into the joint or into the respiratory system or related tissue or lesion is able restore or help restore a normal TEWL value.

As used herein “occlusive” refers to topical formulation or substance, which substantially retards or allows for no or negligible trans-epidermal water loss initially when applied topically as an unbroken layer on healthy skin.

As used herein “partially occlusive” refers to topical formulation or substance, which allows for moderate trans-epidermal water loss initially when applied topically as an unbroken layer on healthy skin.

As used herein a “penetration enhancer” refers to a compound or component of a topical formulation, which increases penetration of active ingredient through the skin barrier or tissue. In some embodiments, a penetration enhancer dissolves a significant proportion of active agent. In some embodiments, a penetration enhancer does not dissolve a significant proportion of active agent. In some embodiments a significant proportion is more than 0.1% by weight of composition.

As used herein, “hydrophilic” refer to a compound or a composition that is miscible with water. In one or more embodiments, a composition may be “hydrophilic” in character even though it may comprise a compound that has some hydrophobic properties.

As used herein “not hydrophilic” refers to a compound or a composition that is not miscible with and/or repels water. In one or more embodiments, a composition may be “not hydrophilic” in character even though it may comprise a compound that has some hydrophilic property.

As used herein “not hydrophobic” refers to a compound or a composition that is not miscible with and/or repels oil. In some embodiments, a composition may be “not hydrophobic” in character even though it may comprise a compound that has some hydrophobic property.

As used herein “free of preservatives” refers to compositions that comprise no or a negligible amount of preservatives. As used herein, “essentially free of preservatives” refers to compositions that comprise less than 0.05% of preservatives by weight. In some embodiments, an essentially preservative-free composition comprises 0.04%, 0.03%, 0.02%, or 0.01% preservatives by weight. The terms “substantially preservative-free” refer to compositions that comprise 3% or less than 3% of preservatives by weight. In some embodiments, the composition comprises less than 2% preservative by weight, or less than 1%, or less than 0.5%, or less than 0.4%, or less than 0.3%, or less than 0.2%, or less than 0.1%, or less than 0.09%, or less than 0.08%, or less than 0.07% or less than 0.06% preservative by weight.

As used herein, free of “anti-oxidants” refers to compositions that comprise no or negligible amount of anti-oxidants. As used herein, essentially free of “anti-oxidants” refers to compositions that comprise less than 0.05% of anti-oxidants. In some embodiments, an essentially anti-oxidant free composition comprises 0.04%, 0.03%, 0.02%, or 0.01% anti-oxidant by weight. In some embodiments, the composition comprises less than 0.04%, or less than 0.03%, or less than 0.02%, or less than 0.01%, or less than 0.005%, or less than 0.001% by weight of anti-oxidant. As used herein, substantially free of “anti-oxidants” refers to compositions that comprises 2% or less than 2% by weight of anti-oxidant. In some embodiments, the composition comprises less than 1.5%, or less than 1%, or less than 0.5%, or less than 0.4%, or less than 0.3%, or less than 0.2%, or less than 0.1%, by weight of anti-oxidant.

As used herein, free of “additional stabilizers” refers to composition that comprises no or a negligible amount of additional stabilizers. As used herein, essentially free of “additional stabilizers” refers to compositions that comprise less than 0.05% of additional stabilizers. In some embodiments, an essentially additional stabilizer-free composition comprises 0.04%, 0.03%, 0.02%, or 0.01% additional stabilizer by weight. In some embodiments, the composition comprises less than 0.04%, or less than 0.03%, or less than 0.02%, or less than 0.01%, or less than 0.005%, or less than 0.001% by weight of additional stabilizer. As used herein, substantially free of “additional stabilizers” refers to compositions that comprises 2% or less than 2% by weight of additional stabilizers. In some embodiments, the composition comprises less than 1.5%, or less than 1%, or less than 0.5%, or less than 0.4%, or less than 0.3%, or less than 0.2%, or less than 0.1%, by weight of additional stabilizers.

As used herein the term “suspended” refers to active agent particles being dispersed in a composition such that less than 0.1% by weight is dissolved within the composition. As used herein, “substantially suspended” refers to active agent particles being dispersed in a composition such that less than 5% by weight is dissolved within the composition. As used herein, “partly suspended” refers to a composition in which a proportion of the active ingredient is dissolved. In some embodiments, the proportion dissolved is at least about 0.1% by weight. In some embodiments, the proportion dissolved is at least about 0.2% by weight. In some embodiments, the proportion dissolved is at least about 0.3% by weight. In some embodiments, the proportion dissolved is at least about 0.4% by weight. In some embodiments, the proportion dissolved is at least about 0.5% by weight. In some embodiments, the proportion dissolved is at least about 0.6% by weight. In some embodiments, the proportion dissolved is at least about 0.7% by weight. In some embodiments, the proportion dissolved is at least about 1% by weight. In some embodiments, the proportion dissolved is at least about 5% by weight. In some embodiments, the proportion dissolved is at least about 10% by weight. In some embodiments, the proportion dissolved is at least about 15% by weight. For clarity, by way of example, the corollary of at least 0.6% dissolved is less than about 99.4% suspended. In some embodiments, having a part dissolved may impact the rate and/or the amount and/or the depth/area of penetration.

As used herein, “a compound that dissolves the active agent” or “a compound that dissolves a proportion of the active agent” refers to a compound that facilitates active agent solubility of more than about 1 mg/g, i.e., more than about 0.1% by weight.

As used herein, “a compound that substantially dissolves the active agent” or “a compound that substantially dissolves a proportion of the active agent” refers to a compound that facilitates active agent solubility of between about 0.1 mg/g to about 1 mg/g i.e., about 0.01% to about 0.1% by weight.

As used herein, “a compound that essentially dissolves the active agent” or “a compound that essentially dissolves a proportion of the active agent” refers to a compound that facilitates active agent solubility of between about 0.01 mg/g to about 0.1 mg/g i.e., about 0.001% to about 0.01% by weight.

As used herein, “a compound that does not dissolve the active agent” or “a compound that does not dissolve a proportion of the active agent” refers to a compound that allows for active agent solubility of less than about 0.01 mg/g, i.e., less than about 0.001% by weight.

As used herein with respect to particle or crystal size, an “average uniform size” refers to average active agent size. The average can be expressed as a proportion of all the particles. Where 90% of the particles or crystals in the suspension are less than Y microns, the D90 is Y microns. In other words, the great majority of particles are smaller than Y microns.

As used herein, “free of agglomerates” refers to a composition in which at least about 95% of the active agent is not present as agglomerates and/or does not form clusters, whilst “substantially free of agglomerates” refers to a composition in which at least about 90% of the active agent is not present as agglomerates and/or does not form clusters.

As used herein, “adhesiveness” refers to the property of a physical attraction and interaction between different surfaces. It can refer to the attraction and interaction of a composition and a surface of an object e.g., body cavity or joint, or it can refer to the attraction and interaction of a compound and a surface of an object e.g., body cavity or joint and may compare it with the competing attraction and interaction of that compound and a composition in which it is suspended.

As used herein, “scavenger” refers to a compound that can capture molecules that promote product degradation. Scavengers can be, for example, but are not limited to free radical scavengers, or aldehyde scavengers.

As used herein a “maintenance application” refers to a topical application of a composition in an amount that can help to sustain a steady-state level of a condition or disorder, or to reduce the possibility of a deterioration of a condition or a disorder, or to prevent a relapse, or return of a condition or a disorder.

As used herein, “injectors” or “injection devices” includes needles, such as 14-28 gauge, other conventional methods, catheters, infusion pumps, pumps, pens, and guns.

As used herein, a joint is a point where two bones can make contact or interrelate.

As used herein, nomenclature for compounds including organic compounds, can be given using common names, IUPAC, IUBMB, or CAS recommendations for nomenclature. One of skill in the art can readily ascertain the structure of a compound if given a name, either by systemic reduction of compound structure using naming conventions, or by commercially available software, such as CHEMDRAW™ (Cambridgesoft Corporation, U.S.A.).

The term “therapeutic index”, also known as the “therapeutic window” or “safety window” defines the relative safety of a drug. The therapeutic index may be calculated as the ratio of the area under the curve (AUC) in blood, at a concentration of drug that results in no toxicity (No Observed Adverse Effect Level—NOAEL), to the concentration of drug that produces the desired efficacy, typically the dose that has a 50% effect—the Effective dose 50 or ED50. TI=AUC(NOAEL)/AUC(ED50).

The term “cyclic” or variants thereof defines a compound, or a substituent group on the compound, in which one or more series of atoms in the compound or substituent is connected to form a ring. The term “acyclic” defines a compound or substituent containing no rings of atoms.

The term “alkyl” is well known in the art and defines univalent groups derived from alkanes by removal of a hydrogen atom from any carbon atom, wherein the term “alkane” is intended to define cyclic or acyclic branched or unbranched hydrocarbons having the general formula CnH2n+2, wherein n is an integer ≥1.

The term “cycloalkyl” defines all univalent groups derived from cycloalkanes by removal of a hydrogen atom from a ring carbon atom. The term “cycloalkane” defines saturated monocyclic and polycyclic hydrocarbons.

The term “alkylol” defines a hydroxy derivative of an alkyl radical, i.e., a hydroxy-alkyl.

The term “halo” is well known in the art and defines a halogen radical that, when bonded to a carbon radical makes a fluoride, chloride, bromide, or iodide compound.

The term “alkyloxy” is synonymous with “alkoxy” and when used herein defines a univalent group comprising an alkyl singly bonded to an oxygen atom, derived from the corresponding alcohol by removal of the hydrogen atom bonded to the oxygen atom.

The term “alkylamino” is synonymous with “alkamino” and, when used herein, defines a univalent group comprising an alkyl singly bonded to an amino group, derived from the corresponding amine by removal of a hydrogen atom bonded to the nitrogen atom.

The term “oxacycloalkyl” defines a univalent group comprising a cycloalkyl, in which one of the CH2 moieties is replaced with an oxide. Similarly, the term “azacycloalkyl” defines a univalent group comprising a cycloalkyl, in which one of the CH2 moieties is replaced with an NH moiety.

The term “stereoisomer” is used herein to refer to isomers that possess identical molecular formulae and sequence of bonded atoms, but which differ in the arrangement of their atoms in space.

The term “enantiomer” defines one of a pair of molecular entities that are mirror images of each other and non-superimposable, i.e., cannot be brought into coincidence by translation and rigid rotation transformations. Enantiomers are chiral molecules, i.e., are distinguishable from their mirror image.

The term “racemic” is used herein to pertain to a racemate. A racemate defines a substantially equimolar mixture of a pair of enantiomers.

The term “diastereoisomers” (also known as diastereomers) defines stereoisomers that are not related as mirror images.

The term “solvate” is used herein to refer to a complex comprising a solute, such as a compound or salt of the compound, and a solvent. If the solvent is water, the solvate may be termed a hydrate, for example a mono-hydrate, di-hydrate, tri-hydrate etc., depending on the number of water molecules present per molecule of substrate.

The term “isotope” is used herein to define a variant of a particular chemical element, in which the nucleus necessarily has the same atomic number but has a different mass number owing to it possessing a different number of neutrons.

The term “prodrug” is used herein to refer to a compound which acts as a drug precursor and which, upon administration to a subject, undergoes conversion by metabolic or other chemical processes to yield a compound of formula (I).

The term “pharmaceutically acceptable excipient” defines substances other than a pharmacologically active drug or prodrug, which are included in a pharmaceutical product.

The term “topical”, when used with respect to compounds or compositions, is used to refer to the ability to apply the compound or composition to body surfaces, for example skin, tissue, a body cavity, mucous membranes, parenchyma, interstitium, alveoli, mucosa, and respiratory systems (through an inhaler or nebulizer). Topical compounds or compositions, in some embodiments, e.g. where administration can be internal and topical, such as by inhalation, may be applied in the form of mist, spray, aerosol, liquid bolus or powder. In some embodiments, e.g., where application is external and topical, they may be applied as foams, creams, gels, lotions, or ointments.

The term “oral”, when used with respect to compounds or compositions, is used to refer to the ability to administer the compound or composition through the mouth. Typically, oral compounds exhibit a systemic effect rather than a topical effect, i.e., they affect multiple organ systems, rather than a local area.

The term “parenteral”, as used herein, includes subcutaneous, intravenous, intramuscular, intra-articular (IA), intra-synovial, intrasternal, intrathecal, intrahepatic, intralesional, intraperitoneal (IP), and intracranial injection or infusion techniques.

As used herein, the term “preventing” or “prophylaxis” refers to avoiding the onset of a disorder or condition from occurring in a subject that has not yet been diagnosed as having the disorder or condition, but who may be susceptible to it.

The terms “transduce” or “transducing”, when used with respect to a signal, are synonymous with “transfer” or “transferring”, i.e., “signal transduction” is the process of transferring a signal throughout an organism, for example, through a cell.

The term “pan” is used herein to refer to “all”. For example, pan inhibition of the BET family means that all of the members of the BET family (BRD2, BRD3, BRD4 and BRDT) are inhibited. For example, pan-BD inhibitor or PAN BET inhibitor means binding to both BDI and BDII.

The term “T-cell” (also known as a T lymphocyte) is known in the art to refer to a lymphocyte with a T-cell receptor on the cell surface (a molecule that is responsible for recognizing fragments of antigen peptides).

The term “cytokine” is used herein to refer to a small protein (˜5 to 20 kDa) that is important in cell signaling, such as autocrine, paracrine and endocrine signaling, as immunomodulating agents.

The term “chemokine” is used herein to refer to a family of cytokines that are able to induce directed chemotaxis in responsive cells, i.e., they act as a chemoattractant to guide the migration of cells.

The term “intrinsic clearance” is well known in the art and refers to the ability of the liver to remove a drug in the absence of flow limitations and binding to cells or proteins in the blood. Intrinsic clearance is herein expressed as a percentage of liver blood flow, i.e.:

$$Intrinsic clearance ( \% ) = \frac{rate of drug clearance}{rate of liver blood flow} \times 100$$

The term “pharmaceutically acceptable salt” is intended to define organic and/or inorganic salts that are pharmaceutically useful. All the compounds described herein above and herein may be in the form of a pharmaceutically acceptable salt. The compounds may be isolated from reaction mixtures as pharmaceutically acceptable salts. Alternatively, the pharmaceutically acceptable salt may be prepared in situ during the final isolation and purification of compounds by reacting a carboxylic acid-containing moiety with a suitable base such as a hydroxide, carbonate or bicarbonate of a pharmaceutically acceptable metal cation, or with ammonia or a primary, secondary or tertiary amine. Pharmaceutically acceptable salts include cations based on alkali metals or alkaline earth metals such as lithium, sodium, potassium, calcium, magnesium and aluminum salts and nontoxic quaternary ammonia and amine cations including ammonium, tetramethylammonium, tetraethylammonium, methylamine, dimethylamine, trimethylamine, triethylamine, diethylamine, and ethylamine. Other examples of organic amines useful for the formation of base addition salts include ethylenediamine, ethanolamine, diethanolamine, piperidine, and piperazine.

The pharmaceutically acceptable salt may also be prepared by treatment of the compound with a suitable acid, for example, hydrogen chloride, hydrogen bromide, hydrogen iodide, sulfuric acid, phosphoric acid, acetic acid, trifluoroacetic acid, propionic acid, glycolic acid, maleic acid, malonic acid, methanesulfonic acid, fumaric acid, succinic acid, tartaric acid, citric acid, benzoic acid and ascorbic acid.

All the compounds may exist in different stereoisomeric and atropisomeric forms. All stereoisomeric forms and mixtures thereof, including enantiomers and racemic mixtures, are included within the scope of the invention. Such stereoisomeric forms include enantiomers and diastereoisomers. Individual stereoisomers of compounds, i.e., associated with less than 5%, preferably less than 2%, and, in particular, less than 1% of the other stereoisomer, are included. Mixtures of stereoisomers in any proportion, for example, a racemic mixture comprising substantially equal amounts of two enantiomers are also included.

Also included are solvates and isotopically-labelled compounds of the invention. Isotopically-labelled compounds are identical to those recited herein, but for the fact that one or more atoms are replaced by an atom having an atomic mass or mass number different from the atomic mass or mass number predominantly found in nature. Examples of isotopes that can be incorporated into compounds of the disclosure include isotopes of hydrogen, carbon, nitrogen, oxygen, sulfur, fluorine, and chlorine, such as 2H, 3H, 13C, 14C, 15N, 180, 170, 35S, 18F, and 36Cl, respectively.

In a further aspect, intermediates suitable for production of compounds for use in the methods of treatment or prevention described herein are included.

Prodrugs of the compounds and compositions for use in the methods of treatment or prevention described herein are also within the scope of the invention. Upon administration to a subject, a prodrug undergoes conversion by metabolic or other chemical processes to yield a compound of the invention.

All amorphous and crystalline forms of the compounds are included for use in the methods of treatment or prevention described herein.

The term “Dry powder inhalers (DPIs)” is intended to define either passive or active devices depending on the source of airflow for powder aerosolization. These devices are further subdivided as single-dose reusable, multidose, and single-use devices.

The term “passive or active device” is intended to define a DPI wherein a patient's inspiration provides the main source of energy to aerosolize the drug powder. Most passive inhalers consist of an air inlet, dispersion chamber, and mouthpiece, but the dispersion mechanisms vary to a large extent. For the traditional passive inhalers, powders are entrained into the dispersion chamber and airflow disperses the powder bed into inhalable aerosols, which can be inhaled by the patient. This process can depend on the balance between interparticulate cohesive/adhesive forces and the de-agglomeration forces generated by the airflow. The fundamental mechanisms of airflow-induced dispersion are generally believed to be the combined effects of air turbulence and powder impaction. Therefore, de-agglomeration enhancers (such as a 3D array of rods, oscillating bead, or impaction grid) are added. Other inhalers may not rely solely on the inspiration airflow to disperse the powder and a mesh-sieving mechanism is applied. During aerosolization, a mesh strip with the powders packed inside the mesh oscillates and beats to push the powder through the small mesh holes, thereby de-agglomerating the powder. Similarly, a fluttering motion driven by airflow provides the major dispersion forces for other devices. The major issue with passive devices is flow-dependent de-agglomeration driven by inspiration. More recent designs have minimized flow dependency, but it remains a potential issue at low flow rates. For patients with compromised lung functions, active devices are more suitable to deliver pharmaceutical aerosols to the lungs.

The term “active device” is intended to define a DPI wherein external energy sources disperse the drug powder; hence, aerosolization efficiency is independent of inspiration. Dispersion can be achieved by compressed air, electrical vibration, or mechanical impeller. This DPI can be particularly critical to the inhalation treatment for patients with limited lung function or much reduced lung volume, such as PF patients.

The term “multidose inhalers” is intended to define multi-unit and reservoir types. In multi-unit DPIs, powders are packed in the individual blister or foil, whereas the dose of the reservoir type is dispensed by a metering valve. The multi-unit devices may offer better protection against moisture and light with an additional separate package for each dose unit. A mechanical or electrical dose counter is essential to indicate the remaining dose.

The term “single-dose reusable devices” is intended to define devices wherein each dose is prepared in a separate single unit such as a capsule or blister. During administration, a single-dose unit is loaded into the inhaler, aerosolized for patient inhalation, and then discarded. Designing the device with the formulation as a separate entity minimizes its dimensions. Additionally, the powder mass in each unit is flexible, permitting use of the same device for both low- and high-dose therapies.

The term “Nebulizers” is intended to define devices that turn liquid medicine into a mist, which is delivered into the lungs. Nebulizers are particularly useful for diseases that require higher pulmonary doses (e.g., PF) and patients who are unable to coordinate or achieve flow rates necessary for use of other inhalation devices (e.g., children). Three main types of nebulizers are available, which are categorized according to the mechanism used to convert the drug solution or suspension into an inhalable aerosol. Jet nebulizers remain the most common (as they are the cheapest and may be provided by healthcare) and use compressed gas to disperse the liquid medication into aerosol droplets. However, treatment times are long, the air compressors are heavy and noisy, and mechanical shear forces can affect certain medications. Alternatively, ultrasonic nebulizers operate silently and are much more portable. The major drawback is a tendency to heat liquid in the reservoir making it inappropriate for thermolabile medications such as proteins. Vibrating mesh nebulizers are the newest technology, which overcome the disadvantages of both jet and ultrasonic nebulizers, with rapid treatment times, minimal residual volume, and greater aerosol delivery. Nonetheless, their high cost remains a barrier to greater patient use. Also, the formulation and particle size distribution should be designed to avoid potential retention on/within the mesh. A principle flaw or drawback of these devices is that aerosol is generated continuously throughout the patient's entire respiratory cycle. Thus, a large proportion of medication is lost during exhalation, resulting in inefficient aerosol drug delivery and variable dosing. This may be in part controlled by mechanical means as seen with breath-enhanced or breath-actuated jet nebulizers that limit the majority of aerosolization to the patient's inspiratory phase or by the coupling of software control with nebulizers. In some embodiments, a jet nebulizer is used. In some embodiments, a mesh nebulizer is used.

It will be appreciated by those skilled in the art that numerous variations and/or modifications may be made to the invention as described herein without departing from the scope of the invention as described. The present embodiments are therefore to be considered for descriptive purposes and are not restrictive and are not limited to the extent of that described in the embodiment. The person skilled in the art is to understand that the present embodiments may be read alone, or in combination, and may be combined with any one or a combination of the features described herein. It should be noted that the abbreviations “ml” and “mL” are used interchangeably herein and both stand for milliliter.

The subject-matter of each patent and non-patent literature reference cited herein is hereby incorporated by reference in its entirety including in respect of its compositions, excipients, methods, and uses.

The compounds and their uses and surprising results are now described in detail. Exemplary compounds useful according to certain embodiments, include, e.g., those described herein and further include, e.g., those as disclosed in patent application PCT/EP2020/061173 filed on 22 Apr. 2020; the disclosure of which is herein expressly incorporated by reference in its entirety and also particularly in respect of the disclosed compounds, their methods of manufacture, and compositions comprising them, including excipients.

As described in PCT/EP2020/061173, many of the compounds and compositions were stable in human skin and under hydrolytic conditions at a range of pH values. Furthermore, formulations of the compounds assumed to be capable of delivering practicable concentrations of the compound into the epidermis of the skin and the compounds were not toxic to skin cells. Some of the compounds and compositions, were ‘soft drugs’, meaning they exhibited surprisingly effective clearance by the liver, offering potential use as medicaments with a lower risk of side-effects. Some of the compounds were selective for BDII over BDI offering the potential of an improved therapeutic index and a lower risk of side-effects while others were pan BET inhibitors which were able to bind to both BDI and BDII.

The present disclosure is directed to pan BET inhibitors, including topical pan BET inhibitors, described above and more specifically to a specific set of compounds as well as their uses in the treatment and/or prophylaxis of selected diseases and disorders that can be treated topically described herein, or, alternatively, of a joint or joint related diseases and disorders that can be treated as described herein.

In one or more embodiments, there is provided one or more compounds and compounds that can function as a “soft” PAN BD BET inhibitor. In one or more embodiments, the one or more compounds that can impact positively on diseases and disorders involving multiple, diverse inflammatory cell signaling pathways. In one or more embodiments, there is provided one or more compounds that are applicable to and can have therapeutic activity, to specific diseases and disorders involving cytokine pathways as part of the etiological factors implicated in the manifestation of the symptoms thereof, for example, to a number of neutrophilic dermatoses such as pyoderma gangrenosum (PG), palmoplantar pustulosis (PPP), and generalized pustular psoriasis (GPP).

In one or more embodiments, there is provided one or more compounds that are applicable to and can have therapeutic activity, to specific pigmentation diseases and disorders where there is pigmentation lightening (e.g., appearance of pale or white patches), for example, vitiligo, chemical leukoderma resulting in linear or splotchy white areas of skin, tinea versicolor spots, albinism where there are lower levels of melanin in the skin, or pityriasis alba which results in lighter patches of skin. Also, for example where there are appearances of pale or white patches, are atrophie blanche, Griscelli syndrome, Halo moles, Hermansky-Pudlak syndrome, Hypomelanosis of Ito, Idiopathic guttate hypomelanosis, Leprosy, Leukoderma, Lichen sclerosus, Lupus erythematosus, Morphoea, Mycosis fungoides, Naevus anaemicus, Naevus depigmentosus, Piebaldism, Pityriasis versicolor, Poliosis, Postinflammatory hypopigmentation, Progressive macular hypopigmentation, Tuberous sclerosis (ashleaf spots), and Waardenburg syndrome. People with vitiligo may be more likely to develop other autoimmune diseases (in which the body's immune system attacks itself), such as hypothyroidism, diabetes, pernicious anemia, Addison's disease, and alopecia areata. Also, people with autoimmune diseases are more at risk for developing vitiligo.

A joint is a point where two bones may make contact or interrelate. Joints can be classified either histologically on the dominant type of connective tissue or functionally based on the amount of movement permitted. Histologically the three types of joints in the body are fibrous, cartilaginous, and synovial. Functionally the three types of joints are synarthrosis (immovable), amphiarthrosis (slightly moveable), and diarthrosis (freely moveable). The two classification schemes correlate: synarthroses are fibrous, amphiarthroses are cartilaginous, and diarthroses are synovial (see Cope P J et al. Models of osteoarthritis: the good, the bad and the promising. Osteoarthritis Cartilage. 2019 February; 27(2):230-239). A fibrous joint is a fixed joint where fibrous tissue comprised primarily of collagen connects bones. Fibrous joints are usually immoveable (synarthroses) and have no joint cavity. They are subdivided further into sutures, gomphoses, and syndesmoses. In cartilaginous joints, the bones attach by hyaline cartilage or fibrocartilage. Depending on the type of cartilage involved, the joints are further classified as primary and secondary cartilaginous joints. Synovial joints are freely mobile (diarthroses) and are considered the main functional joints of the body. Synovial joints are often further classified by the type of movements they permit. There are six such classifications: hinge (elbow), saddle (carpometacarpal joint), planar (acromioclavicular joint), pivot (atlantoaxial joint), condyloid (metacarpophalangeal joint), and ball and socket (hip joint) (see Juneja P. et al., Anatomy, Joints, StatPearls Publishing; 2022, available from: https://www.ncbi.nlm.nih.gov/books/NBK507893/). In some embodiments, the joint is a finger joint, a wrist joint, an elbow joint, an axillary joint, a hip joint, a knee joint, a toe joint, an ankle joint, a shoulder joint, a spinal joint, and/or a jaw joint.

In some embodiments, the one or more compounds can impact positively a joint or joint related disease and disorder involving multiple, diverse inflammatory cell signaling pathways. In some embodiments, there is provided one or more compounds that are applicable to and can have therapeutic activity, to specific joint or joint related diseases and disorders. In some embodiments, there is provided at least one compound applicable to and having therapeutic activity to specific joint or joint related secondary diseases and disorders. In some embodiments, the disclosed compounds and compositions reduce inflammation in the joint and/or in the surrounding tissues. In some embodiments, the joint or joint related diseases and disorders are chosen from arthritis, bursitis, Ehlers-Danlos syndrome, epicondylitis, Felty Syndrome, Sgouty arthritis, psoriatic arthritis, osteoarthritis, rheumatoid arthritis, Sill's disease, tenosynovitis, synovitis, Sjögren's Syndrome, lyme disease, Whipple disease, bone cancer, lupus, and other autoimmune joint disorders. In some embodiments, the disease is rheumatoid arthritis.

In some embodiments, there is provided one or more compounds that are applicable to and can have therapeutic activity, to diseases where there is formation of or increase in scar tissue or fibrosis. In some embodiments, the compounds are applicable to and can have therapeutic activity where the appearance of or increase of scar tissue or fibrosis appears in the lung. In some embodiments, the compounds are applicable to and can have therapeutic activity where the scar tissue or fibrosis is between lung alveoli. In some embodiments, the compounds are applicable to and can have therapeutic activity where the scar tissue or fibrosis damages the alveoli. In some embodiments, the compounds are applicable to and can have therapeutic activity where the scar tissue or fibrosis results in a reduced lung surface area. In some embodiments, the compounds are applicable to and can have therapeutic activity where the scar tissue or fibrosis results in the lung tissue being less elastic, or results in a stiffening of the alveoli. In some embodiments, the scar tissue or fibrosis occurs in PF. In some embodiments, the PF is idiopathic PF or IPF.

In some embodiments, there is provided one or more compounds that are applicable to and can have therapeutic activity, to fibrotic diseases and disorders where there is, for example, aberrant or deregulated wound healing leading to considerable tissue remodeling and the formation of permanent scar tissue, for example, PF.

In one or more embodiments, the present application provides specific soft pan-BD BET inhibitors (hereinafter BETi) which were found to be useful in the treatment or prophylaxis of the conditions described herein.

In some embodiments, the soft pan-BD BET inhibitors are chosen from

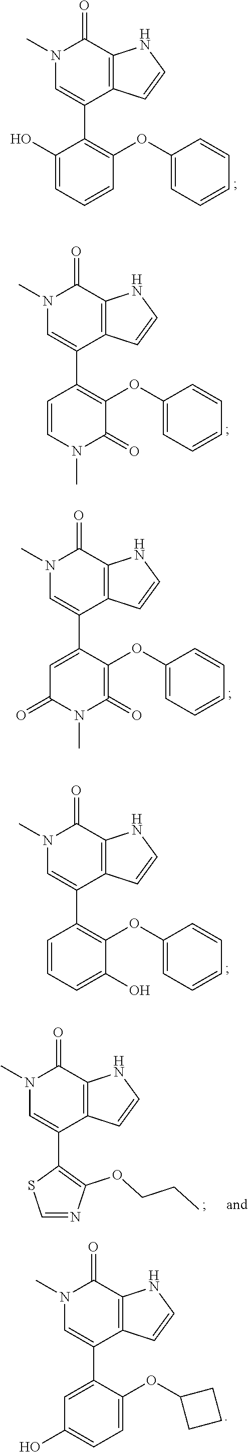

In some embodiments, the present application provides compositions comprising a BETi and a carrier in which the BETi is delivered as a particulate or powder form, which can include crystalline forms. In some embodiments, the present application provides compositions comprising a BETi and a carrier in which the BETi is delivered as a liquid or mist form. In one or more embodiments, the present application provides compositions, such as topical compositions, comprising a BETi and a carrier in which the BETi is suspended or substantially suspended. In some embodiments, the present application provides compositions comprising a BETi and a carrier in which the BETi is dissolved or substantially dissolved. In some embodiments, the present application provides compositions comprising a BETi and a carrier in which the BETi is provided in the form of micelles. In some embodiments, the present application provides compositions comprising a BETi and a carrier in which the BETi is provided in the form of liposome compositions. In some embodiments, the present application provides compositions comprising a BETi and a carrier in which the BETi and carrier are lyophilized. In one or more embodiments, the family of BETi disclosed herein has been found to be surprisingly effective in inhibiting all four BET BRDs at binding domain II (BDII). In one or more embodiments, the BETi described herein may also have an effect in inhibiting BET BRDs at binding domain I (BDI). In some embodiments, the BETi can bind effectively to both BDII and BDI. In one or more embodiments, the BETi show a binding affinity for BDI and BDII and slightly greater selectivity for BDII over BDI. In some embodiments, the BETi compound has a similar binding affinity for BDI and BDII.

In one or more embodiments, the BETi may have a therapeutic effect systemically. In some embodiments, the BETi may be effective therapeutically by topical administration. In some embodiments, e.g., when applied topically, systemic penetration is low and below a therapeutic window. In other embodiments e.g., when applied topically, systemic penetration may be sufficient for the BETi to have a systemic therapeutic effect in addition to any topical effect. In some embodiments, the administration is designed to be intradermal. In some embodiments, the administration is designed to be transdermal. Topical delivery can be by application of the compound or a pharmaceutical composition comprising the BETi to the skin or mucosal surface of a subject, including internal surfaces of body cavities, such as the gastrointestinal tract, vagina, air passageways and lungs. Topical delivery to the air passageways and to the lungs can be by various inhalation methods, including inhalation of powders, solutions, and suspensions. The solutions and suspensions (of the active ingredient in a liquid carrier) may in some embodiments be delivered as a mist inhaled into the lungs. In some embodiments, the administration is designed to be intra-parenchymal or intra-alveoli. In some embodiments, the administration is designed to be trans-parenchymal or trans-alveoli. Types of inhalers include metered-dose inhalers, dry powder inhalers, soft mist inhalers, and nebulizers. Inhalation therapy may be provided. In some embodiments, it may be provided as a short burst of aerosolized compound or composition usually as a metered dose. In some embodiments, it can be a delivered as a spray. In some embodiments, it may be provided over a longer period e.g., 10-20 mins as a mist through a nebulizer.

A key challenge in formulating a nebulizer suitable formulation is in understanding the effect of key formulation parameters such as viscosity, surface tension on droplet aerosol properties and for non-dissolved formulations their interaction with drug particle sizes. For example, lung deposition, particularly in the peripheral airways, is influenced by hygroscopic properties of nebulized aerosols. The droplet size of nebulized aerosols can be greatly reduced with micromolar concentrations of larger pluronic polymers added to the formulations. For newer vibrating mesh nebulizers, viscosity and conductivity are particularly important. Specifically, an increase in both these parameters may reduce aerodynamic size, while increased electrolyte concentrations may enhanced aerosol output. Although some parameters are well-understood, greater understanding is still needed regarding other factors such as how the behavior of non-Newtonian fluids influences nebulizer droplet formation.

In some embodiments, the compound or pharmaceutical composition comprising the compound is administered by injection or infusion, and the injection may be administered as a single injection or as sequential injections.

Compounds which have both the properties of a lower liver clearance rate, and a higher plasma stability may, in one or more embodiments, be more effective systemically. Compounds which have the reverse properties of a higher liver clearance rate, and a lower plasma stability may, in one or more embodiments, be more effective topically or locally. Compounds which fall between the aforesaid properties may, in one or more embodiments, be effective when applied locally, topically, and/or systemically. Also compounds that have one of the properties of a lower liver clearance rate or a higher plasma stability, or, alternatively, of a higher liver clearance rate or a lower plasma stability may, in one or more embodiments, be effective when applied locally, topically, and/or systemically. Compounds which have low selectivity for BDII can be potentially more toxic than compounds with a high selectivity for BDII and visa versa. This can translate, in one or more embodiments, where the potential toxicity is ameliorated or avoided by a high liver clearance and/or lower plasma or systemic stability. In other words, choosing compounds with a higher metabolic liability can surprisingly translate into a therapeutically effective drug when applied topically or locally without corresponding unwanted systemic effects and/or adverse reactions, such as toxicity.

A BET BDI and BDII inhibitor compound disclosed herein, or a pharmaceutical composition comprising the compound, may in one or more embodiments, be chosen for use in the treatment, amelioration, or prophylaxis of any of the non-limiting examples of disorders and diseases described herein.

Treatment or amelioration with the selected BET BDI and BDII inhibitors, and compositions comprising the compounds disclosed herein or salts or stereoisomers thereof (or combinations thereof), in some embodiments, may be effective if applied topically, in some other embodiments, may be effective if applied orally, and, in some further embodiments, may be effective if applied topically and orally. Alternatively, treatment or amelioration with the selected BET BDI and BDII inhibitors, and compositions comprising the compounds disclosed herein or salts or stereoisomers thereof (or combinations thereof), in some embodiments may be effective if applied locally, in some other embodiments may be effective if applied systematically, and in some further embodiments may be effective if applied locally and/or systematically. For respiratory disorders and diseases, delivery topically may be through the air passageways and to the lungs, e.g., through inhalation.

In some embodiments, when applied topically the compounds disclosed herein may be effective where the compound is delivered primarily or substantially into the skin with low levels of transdermal penetration. In some embodiments when applied topically the compounds disclosed herein may be effective where the compound is delivered primarily or substantially transdermally. In some embodiments when applied topically the compounds disclosed herein may be effective where the compound is delivered intradermally and transdermally. In some embodiments the penetration of the compound in the epidermis can be higher than that in the dermis. In some embodiments the penetration of the compound in the dermis can be higher than in the epidermis. In some embodiments the penetration of the compound in the dermis is similar to that in the epidermis. In some embodiments the concentration of the compound per unit volume in the epidermis can be higher than that in the dermis. In some embodiments the concentration of the compound per unit volume in the dermis can be higher than in the epidermis. In some embodiments the concentration of the compound per unit volume in the dermis is similar to that in the epidermis.

In some embodiments, when applied topically, the compounds disclosed herein may be effective where the compound is delivered primarily or substantially into the target cells (e.g., alveoli) with low levels of transcellular penetration into the blood system avoiding or reducing potential systemic effects. In some embodiments, when applied topically to the airways and lungs, the compounds disclosed herein may be effective where the compound delivery is primarily or substantially trans-alveoli or trans mucosal. In some embodiments, when applied topically, the compounds disclosed herein may be effective where the compound is delivered intra-alveoli and trans-alveoli.

In some embodiments, when applied topically, the compounds disclosed herein may be effective where the compound is delivered primarily or substantially into the lung parenchyma (the lung parenchyma comprises a large number of thin-walled alveoli, forming an enormous surface area, which serves to maintain proper gas exchange) with low levels of penetration into the blood system avoiding or reducing potential systemic effects. In some embodiments, when applied topically to the airways and lungs, the compounds disclosed herein may be effective where the compound delivery is primarily or substantially trans-parenchymal. In some embodiments, when applied topically, the compounds disclosed herein may be effective where the compound delivery is intra-parenchymal and trans-parenchymal.

In some embodiments, when applied topically to the skin, the compounds disclosed herein may be effective where the compound is delivered primarily or substantially intradermally. In some embodiments, when applied topically to the skin, the compounds disclosed herein may be effective where the compound is delivered primarily or substantially transdermally. In some embodiments, when applied topically, the compounds disclosed herein may be effective where the compound is delivered intradermally and transdermally. In some embodiments, the penetration of the compound in the epidermis can be higher than that in the dermis. In some embodiments, the penetration of the compound in the dermis can be higher than in the epidermis. In some embodiments, the penetration of the compound in the dermis is similar to that in the epidermis. In some embodiments, the concentration of the compound per unit volume in the epidermis can be higher than that in the dermis. In some embodiments, the concentration of the compound per unit volume in the dermis can be higher than in the epidermis. In some embodiments, the concentration of the compound per unit volume in the dermis is similar to that in the epidermis.

Compositions comprising a compound disclosed herein or salt thereof (or combinations thereof) may in some embodiments be provided via intra-articular administration.

In some embodiments they may be provided by intrasynovial administration. In some embodiments they may be provided by intrabursal administration. In some embodiments they may be provided by intraspinal administration. In some embodiments they may be provided by intra tissue or lesional administration. Compositions comprising a compound disclosed herein or salt thereof (or combinations thereof) may, in one or more embodiments, be administered buccally, by inhalation (e.g., spray), epidural, intradermal, intraperitoneal, intrapulmonary, nasally, orally, parenterally, rectally, sublingually, topically, transdermally, vaginally, or via an implanted reservoir. Compositions comprising a compound disclosed herein or salt thereof (or combinations thereof) may in some embodiments be administered into a vein or artery. Examples of dosage forms for airway or lung parenchymal administration of a compound disclosed herein or salt thereof include solid particles, powders, liquids, suspensions, solutions, mists, aerosols or sprays. Examples of dosage forms for topical or transdermal administration of a compound disclosed herein or salt thereof include creams, drops, lotions, foams, gels, inhalants, mousses, ointments, pastes, patches, powders, solutions, or sprays.

In some embodiments, compositions comprising a compound disclosed herein or salt thereof (or combinations thereof) may be administered to young children. In some embodiments, compositions comprising a compound of the invention or salt thereof (or combinations thereof) may be administered to adolescents or teenagers. In some embodiments, compositions comprising a compound of the invention or salt thereof (or combinations thereof) may be administered to adults.

The potential to be effective against one or more disorders or diseases described herein may, in one or more embodiments, be illustrated by the ability of the BETi to modulate or reduce biomarkers known or implicated, for example, in an inflammatory or immune response, such as cytokines. A non-limiting example where locally acting/administered topical BETi could be applicable in treatment of rare, neutrophilic dermatological indications by inhibition of key immune targets such as IL-1, IL-1β, IL-17, IL-8, IFNγ, IL-36γ, and TNFα. Another non limiting example where a locally acting/administered BETi could be applicable for treatment of migratory action of pro-inflammatory cells, inflammatory cell activation, fibroblast or myofibroblast activation and proliferation, regulation of extracellular matrix (ECM) synthesis and degradation, fibroblast or myofibroblast apoptosis, regulation of AKT/mTOR and SMAD pathways, regulation of pro-fibrotic mediators (e.g. TGF-β), keratinocyte inflammatory responses, differentiation of naïve Th0 cells to Th2 cells in adaptive immunity, allergenic response in diseases or disorders, allergenic diseases or disorders, Th2-mediated inflammatory diseases, pruritus signaling diseases or disorders, scarring disorders and diseases involving formation of or increase in scar tissue, fibrosis diseases and disorders, macrophagic, neutrophilic or dermatological indications by inhibition of key immune targets such as IL-17, IL-17A/F, IL-23, TNFα, IL-1β, IL-6, IL-18, TGFβ, IL-4, IL-5, IL-13, IL-15, IL-22, IL-36γ, IFNγ, or IL 31 or CCL 17, CCL26, CCL2, CCL20, CCL22 or serpin b4 or the reduction in the levels thereof. Alternatively, the potential to be effective against one or more disorders or diseases described herein may in some embodiments be illustrated by the ability of the BETi to modulate or reduce one or more matrix matalloproteinases (e.g., MMP3, and MMP-9). For example, MMP-3 can degrade collagen and can facilitate the recruitment and accumulation of inflammatory cells, and MMP-9 is upregulated in patients with diabetic osteoarthritis.

In some embodiments, the BET inhibitor to be used in the present disclosure is a compound of Formula (I) or a tautomer, stereoisomer or a mixture of stereoisomers, or a pharmaceutically acceptable salt, or hydrate, or deuterated derivative thereof:

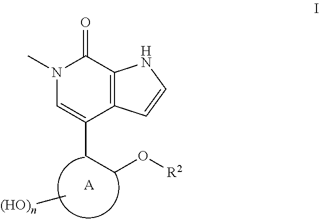

wherein:
ring A is selected from phenyl, N-methyl-2-pyridone, and thiazole;
n is 0 or 1, wherein when A is phenyl, n is 1; when A is N-methyl-2-pyridone, n is 0; and when
A is thiazole, n is 0;
R² is phenyl optionally substituted with 1 to 3 substituents independently selected from hydroxy, halo, C₁-C₆ alkyl, C₃-C₆ cycloalkyl, C₁-C₅ alkyloxy, C₁-C₅ alkylamino, C₁-C₆ fluoroalkyl, C₁-C₅ fluoroalkyloxy, and C₁-C₅ fluoroalkylamino; C₂-C₆ alkyl; and C₃-C₆ cycloalkyl optionally substituted with C₁-C₆ alkoxy.

The compound of clause 1 having the formula I, wherein ring A is selected from

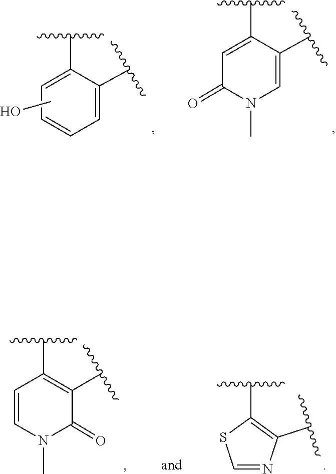

The compound of clause 1 or 2 having the formula I, wherein ring A is selected from

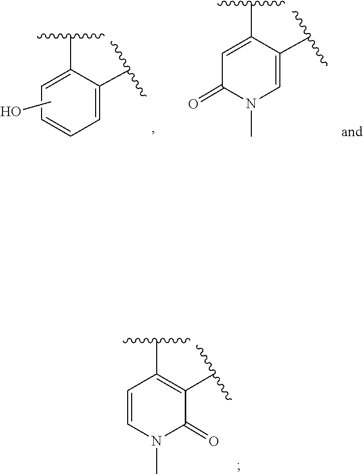

and
R² is phenyl optionally substituted with 1 to 3 substituents independently selected from hydroxy, halo, C₁-C₆ alkyl, C₃-C₆ cycloalkyl, C₁-C₅ alkyloxy, C₁-C₅ alkylamino, C₁-C₆ fluoroalkyl, C₁-C₅ fluoroalkyloxy, and C₁-C₅ fluoroalkylamino.

The compound of any preceding clause having the Formula II, having the structure:

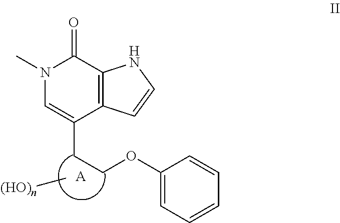

wherein:
ring A is selected from phenyl and N-methyl-2-pyridone; and
n is 0 or 1, wherein when A is phenyl, n is 1; and when A is N-methyl-2-pyridone, n is 0.

The compound of any preceding clause having formula II, wherein ring A is selected from

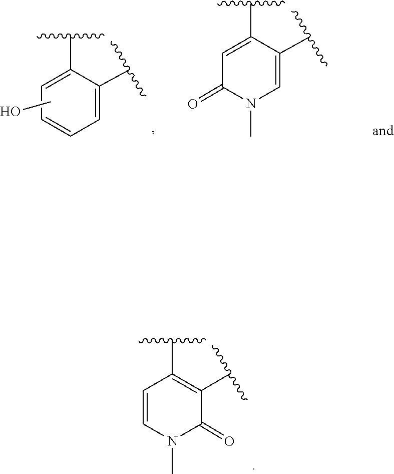

The compound of any preceding clause having the Formula III, having the structure:

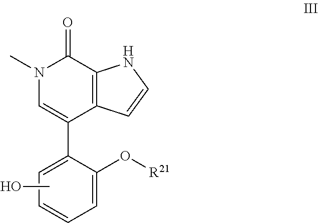

R²¹ is selected from (i) phenyl optionally substituted with 1 to 3 substituents independently selected from hydroxy, halo, C₁-C₆ alkyl, C₃-C₆ cycloalkyl, C₁-C₅ alkyloxy, C₁-C₅ alkylamino, C₁-C₆ fluoroalkyl, C₁-C₅ fluoroalkyloxy, and C₁-C₅ fluoroalkylamino; and (ii) C₃-C₆ cycloalkyl optionally substituted with C₁-C₆ alkoxy.

The compound of any preceding clause having the Formula III, wherein R²¹ is phenyl, cyclobutyl, or cyclohexyl optionally substituted with C₁-C₆ alkoxy. In some embodiments, in Formula III, R²¹ is phenyl.

The compound of any preceding clause having the formula I, wherein the compound has the structure:

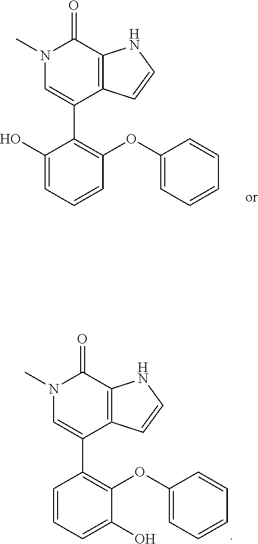 [SMILES: Cn1cc(-c2c(O)cccc2Oc2ccccc2)c2cc[nH]c2c1=O ; Cn1cc(-c2cccc(O)c2Oc2ccccc2)c2cc[nH]c2c1=O]

The compound of any preceding clause having the formula I, wherein the compound has the structure:

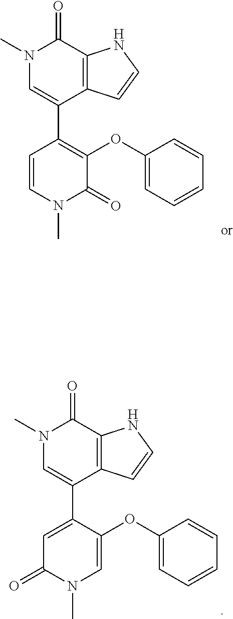 [SMILES: Cn1ccc(-c2cn(C)c(=O)c3[nH]ccc23)c(Oc2ccccc2)c1=O ; Cn1cc(Oc2ccccc2)c(-c2cn(C)c(=O)c3[nH]ccc23)cc1=O]

The compound of any preceding clause having the formula I, wherein the compound has the structure:

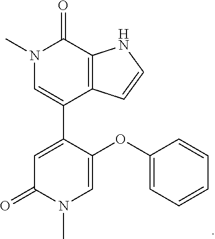 [SMILES: Cn1cc(Oc2ccccc2)c(-c2cn(C)c(=O)c3[nH]ccc23)cc1=O]

In some embodiments, the compound of Formula I is

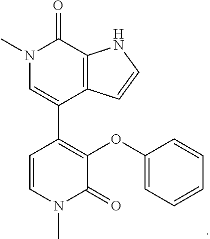 [SMILES: Cn1ccc(-c2cn(C)c(=O)c3[nH]ccc23)c(Oc2ccccc2)c1=O]

The compound of any preceding clause having the formula I, wherein the compound is hereinafter referred to as BETi1 has the structure:

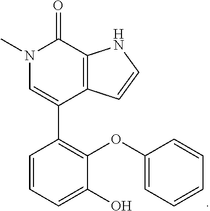 [SMILES: Cn1cc(-c2cccc(O)c2Oc2ccccc2)c2cc[nH]c2c1=O]

In some embodiments, the compound of Formula I is

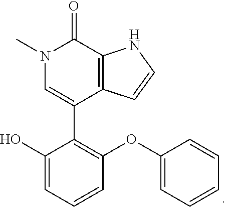 [SMILES: Cn1cc(-c2c(O)cccc2Oc2ccccc2)c2cc[nH]c2c1=O]

The compound of any preceding clause having the formula I, wherein the compound has the structure:

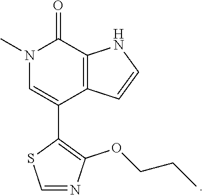 [SMILES: CCCOc1ncsc1-c1cn(C)c(=O)c2[nH]ccc12]

The compound of any preceding clause having the formula I, wherein the compound has the structure:

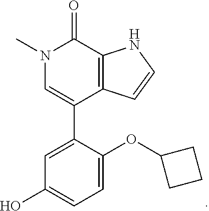 [SMILES: Cn1cc(-c2cc(O)ccc2OC2CCC2)c2cc[nH]c2c1=O]

A pharmaceutical composition comprising any one or a combination of two or more of the compounds in any preceding clause in combination with one or more pharmaceutical excipients for use in the treatment of a disease or disorder disclosed herein.

The pharmaceutical composition of clause 14 having activity against one or more BET domains.

The pharmaceutical composition of clause 15, wherein the BET domains are BDII and BDI.

The pharmaceutical composition of clause 15 or 16, wherein a compound has binding affinity for BDI and BDII and a greater selectivity for BDII. or wherein a compound has a similar binding affinity for BDI and BDII.

The pharmaceutical composition of any of clauses 14 to 17, wherein a compound or two or more compounds have a liver clearance rate of less than 1 hour or less than 45 mins or less than 30 mins or less than 15 mins to be cleared.

The pharmaceutical composition of any of clauses 14 to 17, wherein a compound or two or more compounds have a liver clearance rate of >50% or is >60% or is >70% or is >80% (as % of blood liver flow)

The pharmaceutical composition of any of clauses 14 to 19, wherein a compound or two or more compounds have a plasma stability within 120 mins of less than 50%, or less than 40%, or less than 30% or less than 20%, or less than 10% in 120 mins.

The pharmaceutical composition of any of clauses 14 to 20, wherein a compound or two or more compounds have a plasma stability of <50%, or <45%, <40%, or <35%, or <30% remaining after 120 minutes.

The pharmaceutical composition of any of clauses 14 to 21, wherein BDI and BDII are inhibited and the selectivity of the compound for BDII over BDI is less than threefold.

A method of treatment or prophylaxis of a disorder or disease in a patient in need thereof, wherein the method comprises administering a therapeutically effective amount of a compound according to any of clauses 1 to 13 to the patient, and wherein the disorder or disease is chosen from:
a. skin disorders such as acne, inflammatory acne, acne fulminans, angiofibroma, nodular papulopustular acne, non-inflammatory acne, acne conglobata, acute erysipelas, autoimmune bullous skin disorder such as pemphigus vulgaris (PV) or bullous pemphigoid (BP), bacterial skin infections, viral skin infections, cellulitis, cutaneous abscesses, carbuncles, chronic hand eczema, cutaneous mastocytosis, Dercum disease, dermatological pain, chronic atypical neutrophilic dermatosis with lipodystrophy and elevated temperature (CANDLE), neutrophilic dermatoses, such as pyoderma gangrenosum and Sweets syndrome, paronychial infections, pustulosis palmoplantaris edematous, granuloma annulare, pemphigus, epidermal necrolysis pemphigus, paraneoplastic pemphigus, erythrasma, ecthyma, eczema, folliculitis, furuncles, gustatory sweating, hyperhidrosis, Hailey-Hailey disease, hives, hidradenitis suppurativa, hypertrophic scars, impetigo, ichthyosis, ischemic necrosis, keloids, necrotizing subcutaneous infections, actinic keratosis, keratosis pilaris, miliaria, molluscum contagiosum, lichen planus, netherton syndrome, pityriasis rubra pilaris, prurigo nodularis, pediculosis, pityriasis rosea, scalded skin syndrome, skin rash, trauma or injury to the skin, post-operative or post-surgical skin conditions, wounds, burns (including chemical, electrical fire, friction, radiation, temperature related, thermal and cold), sunburn, scarring, scabies, skin ulcers, urticaria pigmentosa, and warts;
b. a viral disease, a bacterial disease, a yeast disease, for example, if the disease manifests itself in a skin or mucosal disorder;
c. disorders caused by a virus, such as a coronavirus, Epstein-Barr virus (EBV), HIV, HTLV 1, chickenpox, herpes simplex virus infections, herpes zoster virus (VZV), and human papillomavirus (HPV) disease, for example, if the disease manifests itself in a skin or mucosal disorder;
d. an allergic disease, and a food allergy, for example, if the disease or allergy manifests itself in a skin or mucosal disorder;
e. an allograft rejection, a graft-versus-host disease, an allograft rejection reaction, and a graft-versus-host reaction;
f. chilblain lupus, and stimulator of interferon genes-Associated Vasculopathy with onset in Infancy (SAVI);
g. bullous pemphigoid, burns, lichen planus, hidradenitis suppurativa, PG. GPP, PPP, scarring, scar formation, ulcers, skin ulcers, mucosal ulcers, diabetic foot ulcers, wounds, wounds resistant to healing, and wound swelling;
h. a disease or disorder in which a therapeutic effect is achieved by the reduction of TH17 cytokine biomarkers including IL-22, IL-1β, IL17, IL6, IL36 and TNFα or CXCL10 and CCL20 or serpinB4;
i. a disease or disorder in which a therapeutic effect is achieved by the reduction of TH2 cytokine biomarkers including IL-4, IL13, and IL-31 or CCL2, CCL26 and CCL17;
j. restoration of integrity or acceleration of the restoration of the integrity of an area of broken or damaged tissue, skin, or mucosa and in the reduction and amelioration of scar formation or scars;
k. pyoderma gangrenosum (PG), palmar plantar pustulosis (PPP) and generalized pustular psoriasis (GPP);
l. angiofibroma, chronic hand eczema, cutaneous mastocytosis, urticaria pigmentosa, neutrophilic dermatoses such as pyoderma gangrenosum and Sweets syndrome, chronic atypical neutrophilic dermatosis with lipodystrophy and elevated temperature (CANDLE), ichthyosis, keloids, scars, hypertrophic scars, netherton syndrome, prurigo nodularis, and urticaria pigmentosa.
m. Pigmentation diseases and disorders described herein, including vitiligo, chemical leukoderma, tinea versicolor spots, albinism, pityriasis alba, atrophie blanche, Griscelli syndrome, Halo moles, Hermansky-Pudlak syndrome, Hypomelanosis of Ito, Idiopathic guttate hypomelanosis, Leprosy, Leukoderma, Lichen sclerosus, Lupus erythematosus, Morphoea, Mycosis fungoides, Naevus anaemicus, Naevus depigmentosus, Piebaldism, Pityriasis versicolor, Poliosis, Postinflammatory hypopigmentation, Progressive macular hypopigmentation, Tuberous sclerosis (ashleaf spots), and Waardenburg syndrome; and
n. Scar, fibrosis and fibrosis-associated conditions described herein, including PF, said use, in treating one or more of the diseases or conditions listed above, comprising administering to a subject, an effective amount of a compound of the first aspect or specified herein, or a pharmaceutical composition of the second aspect.

The above diseases and disorders may, in one or more embodiments, be associated with the activity of Bromodomain and Extra-Terminal proteins.

The selected BET BDI and BDII inhibitors and pharmaceutical compositions comprising them disclosed herein, may, in one or more embodiments, also be of value and used in the palliation, diagnosis or prevention of any disease, disorder, or condition in humans of one or more of the aforesaid non-limiting examples of disorders and diseases.

A method of treatment or prophylaxis of a disorder or disease in a patient in need thereof, wherein the method comprises administering a therapeutically effective amount of a compound according to any of clauses 1 to 13 to the patient, and wherein the disorder or disease is chosen from bullous pemphigoid, lichen planus, hidradenitis suppurativa, PG, GPP, PPP, scarring, scar formation, ulcers, skin ulcers, mucosal ulcers, diabetic foot ulcers, wounds, wounds resistant to healing, wounds swelling,
or
a disease or disorder in which a therapeutic effect is achieved by the reduction of TH17 cytokine biomarkers including IL-22, IL-1β, IL-17, TL-6, TL-36 and TNFα or CXCL10 and CCL20 or serpinB4
or
a disease or disorder in which a therapeutic effect is achieved by the reduction of TH2 cytokine biomarkers including TL-4, IL-13, IL-17 α, IL22, IL-31, IL-36γ.
or
a disease or disorder in which a therapeutic effect is achieved by the reduction of neutrophil biomarkers including IL-1β, IL-17, TNF-α, IL-8, IL-6 and IL-23,
or
PG, GPP, PPP and wounds.

The method of clause 23 or 24, wherein the disorder or disease is chosen from PG, GPP or PPP.

A method of inhibiting Bromodomain and Extra-Terminal protein activity in a subject comprising administering to a subject an effective amount of any one or a combination of two or more of the compounds defined in any preceding clause or a pharmaceutical composition comprising the compound or compounds in combination with one or more pharmaceutical acceptable excipients.

The method of any of clauses 23 to 26, wherein the subject has a disease or condition associated with the activity of the Bromodomain and Extra-Terminal protein.

The method of clause 27, wherein the compound or pharmaceutical composition comprising the compound is administered topically, locally, or systemically.

The method of clause 28, the method comprises administering a pharmaceutical composition comprising any one or a combination of two or more of the compounds defined in clause 1 in a therapeutically effective amount in combination with one or more pharmaceutical acceptable excipients, and wherein the disorder or disease is chosen from burns, lichen planus, hidradenitis suppurativa, PG, GPP, PPP, scarring, scar formation, ulcers, skin ulcers, mucosal ulcers, diabetic foot ulcers, wounds, wounds resistant to healing, wounds swelling,
or
a disease or disorder in which a therapeutic effect is achieved by the reduction of TH17 cytokine biomarkers including IL-1β, IL17, IL6, 1L36 and TNFα.
or
a disease or disorder in which a therapeutic effect is achieved by the reduction of TH2 cytokine biomarkers including IL-4, IL13, and IL-31.
or
a disease or disorder in which a therapeutic effect is achieved by the reduction of neutrophil biomarkers including IL-1β, IL-17, TNF-α, IL-8, IL-6 and IL-23;
or
PG, GPP, PPP and wounds.

The method of any of the preceding embodiments, wherein the anti-inflammatory effect in preclinical data in mice is similar to a super-potent glucocorticosteroid.

The method of any of the preceding embodiments, wherein the compound appeared well-tolerated in mice, as seen through animal body weight and skin condition.

The method of any of the preceding embodiments, wherein the inhibition of key Th17 or Th2 cytokines in ex vivo data with human skin tissue was stronger when directly compared to JAK1/2 inhibitor, ruxolitinib.

The method of any of the preceding embodiments, wherein the compound significantly reduced expression of several key pro-inflammatory cytokines relevant to Th17 or Th2-mediated immune or autoimmune diseases in an animal model and an ex vivo human tissue study.

The method of any of the preceding embodiments, wherein there is a dose-dependent improvement in the signs and symptoms of inflammation upon administration of the compound or pharmaceutical composition comprising the compound, such as via topical application.

The method of the previous clause, wherein there is a surprising reduction in the dose-dependent improvement in the signs and symptoms of inflammation upon topical application of the compound improvement at high dosage.

The method of any of the preceding embodiments, wherein there is about a 94% reduction, in a mouse study, in the composite inflammation severity score of erythema and scaling, relative to the vehicle control group at treatment day 7.

The method of any of the preceding embodiments, wherein dose-dependent reductions were observed across all treatment groups in Th17-relevant cytokine biomarkers of inflammation, including IL-1β, IL-17, TL-6, TL-36, TNFα and CXC motif chemokine ligand 10 (LP-10).

The method of any of the preceding embodiments, wherein animals (mice) treated topically at all concentrations continued to gain body weight in a similar manner to the healthy control group, whereas animals treated with clobetasol propionate cream 0.05% had a mean body weight loss of approximately 17% compared to the animals treated with BETi1 at 0.1%.

The method of any of the preceding embodiments, wherein no evidence of dermal tolerance issues was noted in animals (mice) to which the compound was applied topically, consistent with the healthy control group, whereas animals treated with clobetasol propionate 0.05% cream had significant skin atrophy as evidenced by deep wrinkling and rhytids (fine wrinkles), marked dermal translucency and loss of elasticity.

The method of any of the preceding embodiments, wherein in an ex-vivo human skin tissue study there is a greater than about 95% inhibition of IL-17, IL-36 and CXCL10 release relative to vehicle control.

The method of any of the preceding embodiments, wherein there is a strong correlation between improvement in clinical severity scores and reduction in many pro-inflammatory biomarkers relevant to Th17-mediated autoimmune diseases.

The method of any of the preceding embodiments, wherein in an ex-vivo human skin tissue study there is a greater than about 95% inhibition of IL-4, IL-13 and IL31 release relative to vehicle control.

The method of any of the preceding embodiments, wherein there is a strong correlation between improvement in clinical severity scores and reduction in many pro-inflammatory biomarkers relevant to Th2-mediated autoimmune diseases.

The method of any of the preceding embodiments, wherein there is a dose-dependent reduction in biomarker expression and wherein BETi1 0.1% demonstrated as having the greatest effect.

The method of any of the preceding embodiments, wherein there is a dose-dependent improvement in the signs and symptoms of pigmentation upon topical application of the compound.

The method of any of the preceding embodiments, wherein there is an increase or a dose-dependent increase in melanin content.

The method of any of the preceding embodiments, wherein there is a decrease or a dose-dependent decrease of MMP-9 secretion.

The method of any of the preceding embodiments, wherein there is a decrease or a dose-dependent decrease in the secretion of E-cadherin.

The method of any of the preceding embodiments, wherein there is an increase or a dose-dependent increase of TRP-1.

The method of any of the preceding embodiments, wherein there is a reduction or a dose-dependent reduction in melanocytorhagy, or wherein upon topical application of the compound the melanocytorhagy is reversed or prevented at least in part, which can be dose-dependent.

The method of any of the preceding embodiments, wherein there is a reversal or a dose dependent reversal of certain gene regulations, wherein one or more of IL1A, IL1B, SOX9, POMC and EDN1 are decreased and/or one or more of WNT16 and RAB3A are increased.

The method of any of the preceding embodiments, wherein there is an improvement, or a dose-dependent improvement in any two or more or all of the following signs and symptoms of pigmentation improvement upon topical application of the compound,
a. wherein there is an increase or a dose-dependent increase in melanin content,
b. wherein there is there is a decrease or a dose-dependent decrease of MMP-9 secretion,
c. wherein there is a decrease or a dose-dependent decrease in the secretion of E-cadherin,
d. wherein there is an increase or a dose-dependent increase of TRP-1.
e. wherein there is a reduction decrease or a dose-dependent reduction in melanocytorhagy, or wherein upon topical application of the compound the melanocytorhagy is reversed or prevented at least in part, which can be dose-dependent, and/or
f. wherein there is a reversal or a dose dependent reversal of certain gene regulations, wherein one or more of IL1A, IL1B, SOX9, POMC and EDN1 are decreased and/or one or more of WNT16 and RAB3A are increased.

The method of any of the preceding embodiments, wherein the disorder or disease includes or is chosen from vitiligo, chemical leukoderma, tinea versicolor spots, albinism, pityriasis alba, atrophie blanche, Griscelli syndrome, Halo moles, Hermansky-Pudlak syndrome, Hypomelanosis of Ito, Idiopathic guttate hypomelanosis, Leprosy, Leukoderma, Lichen sclerosus, Lupus erythematosus, Morphoea, Mycosis fungoides, Naevus anaemicus, Naevus depigmentosus, Piebaldism, Pityriasis versicolor, Poliosis, Postinflammatory hypopigmentation, Progressive macular hypopigmentation, Tuberous sclerosis (ashleaf spots), Waardenburg syndrome. The method of any of the preceding embodiments, wherein the disorder or disease is chosen from a disease or disorder in which a therapeutic effect is achieved by the reduction of cytokine biomarkers including downregulation of one or more of cytokines SOX9, POMC, IL6, IL1a and b, TNF and EDN1 (vasoconstriction) and upregulation of genes related to the WNT/β-catenin pathway and/or also RAB3A. WNT/β-catenin pathway is one of the main pathways known to be altered in vitiligo and leading to the activation of MITF (a key transcription factor involved in the melanocytes differentiation, growth and survival). The method of any of the preceding embodiments, wherein the upregulation of genes related to the WNT/β-catenin pathway includes WNT16. The method of any of the preceding embodiments, wherein other genes of the melanocyte biology are also impacted with some regulations in favor and others in disfavor of the melanogenesis process. The method of any of the preceding embodiments, wherein the vehicle does not have a major effect on gene expression. The method of any of the preceding embodiments, wherein treatment with the vehicle results in induction of GADD45A gene, a marker of DNA damage and the downregulation of WNT16.

In some embodiments, there is an improvement in at least one symptom of the fibrosis or respiratory disorder or disease upon topical application of a BETi solution (e.g. BETi1). In some embodiments, there is an improvement in at least one symptom of the fibrosis or respiratory disorder or disease upon topical application of a BETi suspension or part suspension (e.g., BETi1). In some embodiments, the symptom of fibrosis or respiratory disorder or disease is accumulation of collagen in the lung which is expressed as high levels of hydroxyproline. In some embodiments, an improvement in symptoms is expressed as a reduction in hydroxyproline levels. In some embodiments, a reduction in hydroxyproline levels is observed in subjects (e.g., mice) treated with a BETi, e.g., a BETi1 solution (e.g., at 0.06 mg/mL, 0.6 mg/mL and 3 mg/mL) in comparison to animals treated with vehicle. In some embodiments, reduction in hydroxyproline levels in subjects (e.g., mice) treated with a solution comprising BETi1 (e.g., at 3 mg/mL) is statistically significant compared to subjects treated with vehicle.

The method of any of the preceding embodiments, wherein there is an improvement in one or more of the signs and symptoms of fibrosis upon topical application of a composition comprising the BETi compound, e.g., BETi1. The method of any of the preceding embodiments, wherein there is a retardation or slowing in the development of signs and symptoms of fibrosis upon topical application of the compound. The method of any of the preceding embodiments, wherein there is a reduction in the appearance of scar tissue upon topical application of the compound. The method of any of the preceding embodiments, wherein there is a reduction in the appearance of one or more of the following: alveolar epithelial cell injury, areas of type II cell hyperplasia, accumulation of fibroblasts and myofibroblasts, or the deposition of extracellular matrix proteins upon topical application of the compound. The method of any of the preceding embodiments, wherein there is a reduction in the appearance of scar tissue between lung alveoli upon topical application of the compound. The method of any of the preceding embodiments, wherein there is an increase in the appearance of lung alveoli surface area upon topical application of the compound. In one or more embodiments the improvement or retardation is multifactorial and in part dependent on the amount of the dose, in part on the delivery vehicle composition and form, in part on the method of delivery, and in part on the length of time the airways and lungs are exposed to the dose. In some embodiments, the effect of the dose may be more beneficial where it is given in lower amounts and/or for shorter periods. In some embodiments, the effect of the dose may be less beneficial where it is given in high amounts and/or for too long a period.

In one or more embodiments, histology is the primary data indicating a reduction or amelioration of fibrosis followed by or coupled with the hydroxyproline data. Other data like body weight is informational. Survival endpoint is affected by significant variability and bias has an inherent limitation that even if treatment is being effective the bleomycin induced damage may have already been fatal.

Histology is a picture or snapshot of the absence or presence of fibrosis. The histology slides for example of bleomycin induced lung treated with BETi e.g., BETi1 administered at 0.06 mg/mL compared to bleomycin induced treated with vehicle control showed significant areas of normal lung parenchyma with open airways and open spaces, normal conducting airways as well as other areas with less severe localized fibrosis both in the regions of parenchyma and in the region of the airway. Other groups with higher concentrations of BETi also appeared to lessen Bleo induced fibrosis but to a lesser degree. Such observations are very encouraging and indicate that the BETi compounds can offer a useful method of treating, retarding or ameliorating PF in humans. The histology was scored according to the Ashcroft scale. The best two groups (0.06 mg/mL and 0.6 mg/mL) showed a reduction in fibrosis in the subjects of over about 50% and nearly about 30% within the 20 day study. This may potentially translate over time into a significant delaying or retardation of the disease and a significantly increased life span for affected subjects. In one or more embodiments, the subject's life span may be increased by about 3 months, or 6 months or 9 months or a year or 18 months or two years or longer.

Hydroxyproline is a significant diagnostic biomarker of fibrosis. The method of any of the preceding embodiments, wherein there is a decrease in lung hydroxyproline content levels in bleomycin induced animals treated with a BETi e.g., BETi1 compared to bleo/vehicle. The method of any of the preceding embodiments, wherein there is a statistically significant decrease in lung hydroxyproline content levels in bleomycin induced animals treated with a BETi e.g., BETi1 compared to bleo/vehicle. The method of any of the preceding embodiments, wherein there is a decrease or a dose-dependent decrease in lung hydroxyproline content levels in bleomycin induced animals treated with a BETi e.g., BETi1, wherein lower levels of hydroxyproline are observed with all the doses of the BETi e.g., BETi1 (0.006, 0.6 and 3 mg/mL). The method of any of the preceding embodiments, wherein a higher reduction is observed with the higher dose of the BETi, e.g., BETi1 (e.g., 3 mg/mL). The method of any of the preceding embodiments, wherein the decrease in lung hydroxyproline content levels in bleomycin induced animals treated with the BETi, e.g., BETi1, the observed with 0.06 mg/mL are nearly as low as those observed with the higher dose of 3 mg/mL. The method of any of the preceding embodiments, wherein there is a statistically significant decrease in lung hydroxyproline content levels in bleomycin induced animals treated with BETi1 at 3 mg/mL compared to bleo/vehicle. The method of any of the preceding embodiments, wherein BETi1 at 3 mg/mL is highly statistically different to bleomycin induced animals treated with vehicle control in preventing the production of lung hydroxyproline. The method of any of the preceding embodiments, wherein lung hydroxyproline content levels in bleomycin induced vehicle treated animals are significantly higher than in sham/vehicle administered animals indicating that pulmonary fibrosis has occurred. The method of any of the preceding embodiments, wherein there is an increase or a dose-dependent increase in survival rate in bleomycin induced animals treated with BETi1, wherein higher survival probability occurs with higher doses (e.g., 0.6 and 3 mg/mL). The method of any of the preceding embodiments, wherein hydroxyproline content is reduced by at least about 10%, at least about 20%, at least about 30%, at least about 40%, at least about 50%, or at least about 60%. The method of any of the preceding embodiments, wherein hydroxyproline content is reduced by about 10% to about 50% or by about 15% to about 45%.

The method of any of the preceding embodiments, wherein the bleomycin induction can be fatal to a substantial proportion of the animals as the study progresses. For example, in the bleomycin induced untreated control animals a fatality occurred after a day. After about 10 days and thereafter the two highest BETi groups showed better survival rates whilst the lower concentration had a survival probability not dissimilar to the bleomycin induced vehicle treated control. The method of any of the preceding embodiments, wherein animals receiving higher BETi (e.g., 0.6 and 3 mg/mL doses of BETi) have a higher probability of survival in comparison with the bleomycin vehicle treated control group. The method of any of the preceding embodiments, wherein animals receiving higher doses of BETi (e.g., 0.6 and 3 mg/mL of BETi) have a higher probability of survival from about day 10 or from about day 11 or from about day 12 to the end of the study in comparison with the bleomycin vehicle treated control group. On the other hand, subjects with PF would not have to contend with the effects of bleomycin and may therefore cope better with the disorder in the absence of bleomycin.

The method of any of the preceding embodiments, wherein bleo/vehicle show a greater pathology severity for interstitial fibrosis and subacute interstitial inflammation than sham/vehicle treated group. The method of any of the preceding embodiments, wherein compared to the lung samples from sham/vehicle group, significant increases in both interstitial fibrosis and inflammation are observed in the lung samples of bleo/vehicle treated group. The method of any of the preceding embodiments, wherein fibrosis is characterized by increased numbers of fibroblasts and collagen fibers in the lung interstitium. The method of any of the preceding embodiments, wherein PSR staining severity generally correlates with the mean severity of interstitial fibrosis. The method of any of the preceding embodiments, wherein inflammation is characterized by infiltrates of lymphocytes and macrophages with occasional neutrophils in the lung interstitium. The method of any of the preceding embodiments, wherein bleomycin induced animals treated with a BETi e.g., BETi1 have a lower pathology severity for interstitial fibrosis and subacute interstitial inflammation than observed in animals treated with the bleomycin induced vehicle treated control. The method of any of the preceding embodiments, wherein bleomycin induced animals treated with a BETi e.g., BETi1 with the lowest pathology severity for interstitial fibrosis and subacute interstitial inflammation is observed in animals treated with BETi1 0.06 mg/mL. The method of any of the preceding embodiments, wherein there is a decrease in pathology severity for interstitial fibrosis and subacute interstitial inflammation in animals treated with the BETi compounds described herein, e.g., BETi1. The method of any of the preceding embodiments, wherein there is a decrease in animals treated with the compounds described herein, e.g., BETi1 in the numbers of fibroblasts and collagen fibers in the lung interstitium. The method of any of the preceding embodiments, wherein upon topical application of the BETi compounds described herein, e.g., BETi1 there is a decrease in infiltrates of lymphocytes, macrophages and neutrophils in the lung interstitium, or wherein upon topical application of the BETi compounds described herein, e.g., BETi1 the infiltrates of lymphocytes, macrophages and neutrophils in the lung interstitium is reversed, retarded, or prevented at least in part, and which can be dose-dependent.

The method of any of the preceding embodiments, wherein treatment with BETi1 at 0.06 mg/mL demonstrates normal lung parenchyma with open airways and airspaces and small conducting airways. The method of any of the preceding embodiments, wherein treatment with BETi1 at 0.06 mg/mL results in less severe localized fibrosis in parenchyma and less severe localized fibrosis in airway.

The method of any of the preceding embodiments, wherein mean score measured according to Hübner-modified Ashcroft scale of 0-8 per group for uninduced vehicle is 1.52, for bleomycine-induced vehicle is 6.36, for bleomycine-induced\0.06 mg/mL is 3.00, for bleomycine-induced\0.6 mg/mL is 4.64, and for bleomycine-induced\0.3 mg/mL is 5.52. The method of any of the preceding embodiments, wherein the Ashcroft scoring translates into reductions of about 53% and of about 27% for the 0.06 mg/mL and the 0.6 mg/mL groups respectively when compared to the bleomycin induced vehicle control. The method of any of the preceding embodiments, wherein significant differences are observed when the following groups were compared: uninduced\vehicle vs. bleomycine-induced\vehicle, bleomycine-induced vehicle vs. bleomycine-induced\0.06 mg/mL, and bleomycine-induced\vehicle vs. bleomycine-induced\0.6 mg/mL (*P≤0.05) and bleomycine-induced\vehicle vs. bleomycine-induced\0.3 mg/mL trended toward significance but was not significantly different at P<0.05). The method of any of the preceding embodiments, wherein bleomycine-induced\0.06 mg/mL group has the greatest inhibitory effect on fibrotic tissue formation in the lung.

The method of any of the preceding embodiments, wherein treatment with BETi1 reverses pulmonary fibrosis in a bleomycin-induced animal treated with the BETi compounds described herein, e.g., BETi1. The method of any of the preceding embodiments, wherein mice are given bleomycin for one day to establish pulmonary fibrosis, and then treated daily with the BETi compounds described herein, (e.g., BETi1) and bleomycin concomitantly e.g., until day 21. The method of any of the preceding embodiments, wherein vehicle treated bleomycin induced mice have pulmonary fibrosis with widespread collagen deposition.

The method of any of the preceding embodiments, wherein the BETi compounds described herein (e.g., BETi1) are capable of reversing, retarding or preventing in part fibrotic remodeling in the lungs and/or reversing, retarding or preventing in part deterioration of or damage to normal lung architecture and/or unwanted collagen deposition. The method of any of the preceding embodiments, wherein bleomycin induced mice that receive BETi1 have closer to normal lung architecture at 21 days and/or less new collagen deposition. The method of any of the preceding embodiments, wherein the BETi compounds described herein (e.g., BETi1) are capable of reversing, retarding or preventing in part bleomycin induced fibrotic remodeling in the lungs. The method of any of the preceding embodiments, wherein BETi1 is capable of reversing, retarding or preventing in partbleomycin induced fibrotic remodeling in the lungs. The method of any of the preceding embodiments, wherein the BETi compounds described herein (e.g., BETi1) have a role in the regulation of redox balance in activated myofibroblasts. The method of any of the preceding embodiments, wherein the BETi compounds described herein (e.g., BETi1) have a role in downregulating the secretion of TGFβ. The method of any of the preceding embodiments, wherein the BETi compounds described herein (e.g., BETi1) have a role in downregulating and suppressing transcription of collagen and/or fibronectin genes. The method of any of the preceding embodiments, wherein the BETi compounds described herein (e.g., BETi1) are able to suppress the migratory action of pro-inflammatory cells and inflammatory cells e.g., by targeting CD4+Th17 which secrete IL17A/F. The method of any of the preceding embodiments, wherein the BETi compounds described herein (e.g., BETi11) reduce inflammation. The method of any of the preceding embodiments, wherein the BETi compounds described herein (e.g., BETi1) reduces, prevent or reverse TGF-β-mediated myofibroblast differentiation.

The method of any of the preceding embodiments, wherein the BETi compounds described herein (e.g., BETi1) reduce or reduce in a dose-dependent way TGF-β-mediated myofibroblast differentiation, or wherein the BETi compounds described herein (e.g., BETi1) reduce, reverse, retard, or prevent at least in part fibrosis, which reduction can be dose-related.

The method of any of the preceding embodiments, wherein there is a reversal or a dose related reversal of certain gene regulations, wherein one or more of TL17A/F, TGFβ, collagen and fibronectin genes or other genes involved in TGF-β-mediated myofibroblast differentiation are reversed.

The method of any of the preceding embodiments, wherein there is an improvement in any two or more or all of the following signs and symptoms of fibrosis upon topical application of a BETi compound described herein e.g., BETi1, via inhalation wherein there is an increase oxygen saturation,
wherein there is there is a decrease lung hydroxyproline content levels,
   wherein there is a decrease in pathology severity for interstitial fibrosis and subacute interstitial inflammation,
   wherein there is a decrease in the numbers of fibroblasts and collagen fibers in the lung interstitium,
   wherein there is a reduction in infiltrates of lymphocytes, macrophages and neutrophils in the lung interstitium, or wherein upon topical application of BETi1 the infiltrates of lymphocytes, macrophages and neutrophils in the lung interstitium is reversed or prevented at least in part, and/or
   wherein there is a down regulation of expression of certain gene, chosen from one or more of IL17A/F, TGFβ, collagen and fibronectin genes or other genes involved in TGF-β-mediated myofibroblast differentiation.

The method of any of the preceding embodiments, wherein the compound is able to bind and inhibit BDI and BDII in the lung tissue and upper mucosa, with some selectivity (e.g., <about 10) for BDII, also having a high liver clearance rate and low or no tolerance issues.

The method of any of the preceding embodiments, wherein the compound or pharmaceutical composition comprising the compound is administered by injection or infusion, wherein the injection may be administered as a single injection or as sequential injections.

The method of any of the preceding embodiments, wherein the injection or infusion is into the epidural space or other spinal space, foramenal space, intraarticular space, lesions, periarticular space, perineum space, soft tissues, or the at or near the location of pain and/or damage.

The method of any of the preceding embodiments, wherein the joint or joint related disorder or disease is chosen from arthritis, bursitis, Ehlers-Danlos syndrome, epicondylitis, Felty Syndrome, gouty arthritis, psoriatic arthritis, osteoarthritis, rheumatoid arthritis, Still's disease, tenosynovitis, synovitis, Sjögren's Syndrome, lyme disease, Whipple disease, bone cancer, lupus or other autoimmune joint disorders.

The method of any of the preceding embodiments, wherein the disorder or disease is chosen from one in which a therapeutic effect is achieved by the modulation and/or reduction of one or more cytokines.

The method or use of any of the preceding embodiments, wherein the compound and/or the pharmaceutical composition is well-tolerated.

The method of any of the preceding embodiments, wherein the compound is able to bind and inhibit BDI and BDII in the skin, with some selectivity for BDII, having a high liver clearance rate and low or no dermal tolerance issues.

The method of any of the preceding embodiments, wherein the compound is able to reduce swelling effectively compared to vehicle.

The use of any one of the preceding embodiments, wherein the regulation of key cytokines in ex vivo data with reconstituted skin tissue is stronger when directly compared to JAK1/2 inhibitor, ruxolitinib, which—in the probe of gene expression in vitro—downregulates some inflammatory cytokines but does not have a material effect on WNT/β-catenin pathway or EDN1.

The use of any one of the preceding embodiments, wherein the compound is capable of significant downregulation of one or more (e.g., all) of SOX9, POMC, IL-6, IL1a and b, TNF and EDN1 (vasoconstriction) and upregulation of the WNT pathway (e.g., WNT 16) and/or also RAB3A in an ex vivo reconstituted tissue.

The use of any one of the preceding embodiments, wherein there is a dose-dependent improvement in the signs and symptoms of the pigmentation disorder or disease upon topical application of the compound.

The use of any one of the preceding embodiments, wherein the pigmentation disease or disorder is vitiligo.

The use of any one of the preceding embodiments, wherein there is a dose-dependent reduction in one or more cytokine biomarkers. In some embodiments, the one or more cytokine biomarkers are chosen from IL6, IL1a and b, SOX9, POMC TNF, and/or EDN1 (vasoconstriction). The use of any one of the preceding embodiments, wherein there is a dose-dependent increase in one or more cytokine biomarkers. In some embodiments, the one or more cytokine biomarkers are chosen from WNT16 and RAB3A.

The use of any one of the preceding embodiments, wherein the administration of

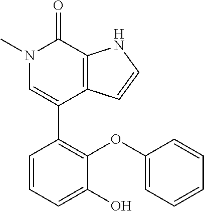 [SMILES: Cn1cc(-c2cccc(O)c2Oc2ccccc2)c2cc[nH]c2c1=O]

reduces melanocytorhagy. In some embodiments,

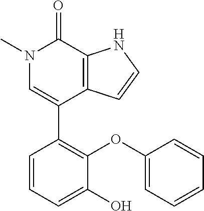 [SMILES: Cn1cc(-c2cccc(O)c2Oc2ccccc2)c2cc[nH]c2c1=O]

has a concentration of about 0.001% to about 1% by weight, or about 0.01% to about 1% by weight of the pharmaceutical composition. In some embodiments, the concentration of

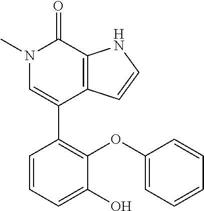 [SMILES: Cn1cc(-c2cccc(O)c2Oc2ccccc2)c2cc[nH]c2c1=O]

is about 0.1% by weight of the composition. In some embodiments, the concentration of

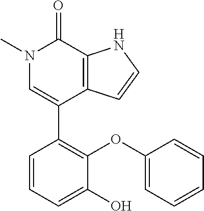 [SMILES: Cn1cc(-c2cccc(O)c2Oc2ccccc2)c2cc[nH]c2c1=O]

is about 1% by weight of the composition.

The use of any one of the preceding embodiments, wherein the BET inhibitor is administered in an amount sufficient to reduce secretion of MMP9 by keratinocytes relative to stimulated vehicle-treated reconstituted human epithelium (RHE).

The use of any one of the preceding embodiments, wherein MMP9 secretion is reduced by at least about 50% relative to stimulated vehicle control and/or numerically superior to Ruxolitinib 1.5%.

The use of the preceding embodiment, wherein MMP9 secretion is reduced by at least about 60%, or at least about 70%, or at least about 80%, or at least about 90%.

The use of the preceding embodiment, wherein MMP9 secretion is reduced by at least about 95%.

The use of any one of the preceding embodiments, wherein the BET inhibitor is administered in an amount sufficient to reduce soluble E-cadherin, relative to a stimulated and vehicle-treated RHE. The use of the preceding embodiment, wherein the BET inhibitor result in a reduction of E-cadherin secretion of at least about 25%, or at least about 30%, at least about 35%, or at least about 40%, or at least about 45%, or at least about 50% compared to stimulated control The use of the preceding embodiment, wherein the BET inhibitor result in a reduction of E-cadherin secretion of 80% or less compared to stimulated control.

The use of the preceding embodiment, wherein the amount of the BET inhibitor ranges from about 0.001% to about 1% by weight of a pharmaceutical composition. In some embodiments, the amount of the BET inhibitor ranges from about 0.1% to about 1% by weight of a pharmaceutical composition. In some embodiments, the amount of the BET inhibitor ranges from about 0.05% to about 5% or from about 0.075% to about 4%, or from about 0.1% to about 3%, or from about 0.125% to about 2.5%, or from about 0.15% to about 2% by weight of a pharmaceutical composition.

The use of any one of the preceding embodiments, wherein the administration of the BET inhibitor results in upregulation of WNT pathway. In some embodiments, the WNT pathway is upregulated by about >50%, >60%, >70%, >75%, >80%, >85%, >90%, >95%, >96%, >97%, >98%, or >99% after administration of the BET inhibitor. In some embodiments WNT16 and/or RAB3A is upregulated by about >50%, >60%, >70%, >75%, >80%, >85%, >90%, >95%, >96%, >97%, >98%, or >99% after administration of the BET inhibitor.

The use of any one of the preceding embodiments, wherein the administration of the BET inhibitor results in downregulation of EDN1.

The use of any one of the preceding embodiments, wherein the administration of the BET inhibitor results in downregulation of one or more or all of 1L6, IL1a, IL1b, and/or TNF. In some embodiments, the levels of one or more or all of SOX9, POMC TL6, IL1a, IL1b, TNF, and/or EDN1 are reduced by about >60%, >70%, >75%, >80%, >85%, >90%, >95%, >96%, >97%, >98%, or >99% after administration of the BET inhibitor.

The method of any of the preceding embodiments, wherein the disorder or disease is chosen from a disease or disorder in which a therapeutic effect is achieved by the reduction of cytokine biomarkers including downregulation of one or more of cytokines of IL17A/F, TGFβ, collagen and fibronectin genes and upregulation or downregulation of other genes related to the fibrosis and inflammation pathway. The fibrosis TGFβ regulated pathway is one of the main pathways known to be altered in PF and leading to TGF-β-mediated myofibroblast differentiation. The method of any of the preceding embodiments, wherein the downregulation of genes related to the fibrosis TGFβ regulated pathway includes collagen and fibronectin. The method of any of the preceding embodiments, wherein other genes of the myofibroblast differentiation pathway are also impacted with some regulations in favor and others in disfavor of the myofibroblast differentiation process. The method of any of the preceding embodiments, wherein the vehicle does not have a major effect on gene expression.

The use of any of the preceding embodiments, wherein the fibrosis or respiratory disorder or disease comprises a lung disorder chosen from a pulmonary fibrosis (PF), idiopathic pulmonary fibrosis (IPF), desquamative interstitial pneumonia (DIP), acute interstitial pneumonia (AIP), nonspecific interstitial pneumonia (NSIP), respiratory bronchiolitis-associated interstitial lung disease (RB-ILD), cryptogenic organizing pneumonia (COP), and lymphoid interstitial pneumonia (LIP).

The use of any of the preceding embodiments, wherein the fibrosis or respiratory disorder or disease is a PF and wherein the PF is chosen from one or more of environmental PF, occupational PF, drug-induced PF, radiation-induced PF, autoimmune lung disease, and idiopathic PF.

The use of any of the preceding embodiments, wherein there is a reduction in at least one symptom of the fibrosis or respiratory disorder or disease upon topical application of about 0.001% to about 0.1% of the BET inhibitor.

The use of any one of the preceding embodiments, wherein at a therapeutically effective amount and dosing period the compound and/or the pharmaceutical composition is well-tolerated.

The pharmaceutical composition of any of the preceding embodiments, wherein there is a reduction in at least one symptom of the fibrosis or respiratory disorder or disease.

The use of any of the preceding embodiments, wherein there is a reduction in at least one symptom of the fibrosis or respiratory disorder or disease upon topical application of about 0.001% to about 0.1% of the BET inhibitor. The use of any one of the preceding embodiments, wherein the BET inhibitor is about 0.001% to about 0.06%, about 0.002% to about 0.05%, about 0.0025% to about 0.01%, or about 0.003% to about 0.008% by weight of the pharmaceutical composition.

The use of any one of the preceding embodiments, wherein the fibrosis or respiratory disease or disorder is PF.

The use of any one of the preceding embodiments, wherein the symptom is a reduction of hydroxyproline levels in bleomycin induced subjects (e.g., mice) treated with a BETi1 solution (e.g., at about 0.06 mg/mL, about 0.6 mg/mL and about 3 mg/mL) in comparison to bleomycin induced subjects treated with vehicle.

The use of any one of the preceding embodiments, wherein the symptom is a reduction of hydroxyproline levels and wherein the reduction in bleomycin induced subjects (e.g., mice) treated with a BETi1 solution at 3 mg/mL is statistically significant in comparison to bleomycin induced subjects treated with vehicle.

The use of any one of the preceding embodiments, wherein the solution is inhaled at least once daily.

The use of any one of the preceding embodiments, wherein the administration of

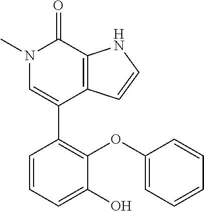 [SMILES: Cn1cc(-c2cccc(O)c2Oc2ccccc2)c2cc[nH]c2c1=O]

reduces fibrosis in the lung.

The use of the preceding embodiment, wherein the fibrosis is less severe in comparison to bleomycin induced subjects treated with vehicle.

The use of any one of the preceding embodiments, wherein the fibrosis is less severe in the parenchyma.

The use of any one of the preceding embodiments, wherein the fibrosis is less severe in the area of the lung airways.

The use of any one of the preceding embodiments, wherein there are areas of normal lung parenchyma with open airways.

The use of any one of the preceding embodiments, wherein the areas of normal lung parenchyma with open airways are substantial.

The use of any one of the preceding embodiments, wherein the areas of normal lung parenchyma with open airways are >about 10%, >about 15%, >about 20%, >about 25%, >about 30%, or >about 35%, >about 40%, >about 45%, >about 50%, or >about 55% of the lung.

The use of any one of the preceding embodiments, wherein the areas of normal lung parenchyma with open airways are about 10% to about 60% of the lung.

The use of any one of the preceding embodiments, wherein there is a reduction in Ashcroft score in comparison to bleomycin induced subjects treated with vehicle.

The use of the preceding embodiment, wherein the reduction is >about 10%.

The use of any one of the preceding embodiments, wherein the reduction is >about 25%.

The use of any one of the preceding embodiments, wherein the reduction is >about 50%.

The use of any one of the preceding embodiments, wherein the reduction is about 10% to about 60% or is about 12% to about 54%.

The use of any one of the preceding embodiments, wherein the reduction is a mean.

The use of any one of the preceding embodiments, wherein

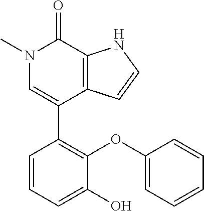 [SMILES: Cn1cc(-c2cccc(O)c2Oc2ccccc2)c2cc[nH]c2c1=O]

has a concentration of about 0.001% to about 0.1% by weight of the pharmaceutical composition.

The use of the preceding embodiment, wherein

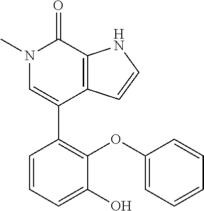 [SMILES: Cn1cc(-c2cccc(O)c2Oc2ccccc2)c2cc[nH]c2c1=O]

is about 0.001% to about 0.06% by weight of the pharmaceutical composition.

The use of any one of the preceding embodiments, wherein the concentration of

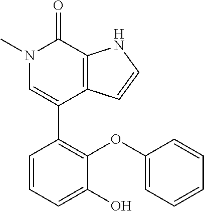 [SMILES: Cn1cc(-c2cccc(O)c2Oc2ccccc2)c2cc[nH]c2c1=O]

is about 0.006% or less than about 0.006% by weight of the pharmaceutical composition.

The use of any one of the preceding embodiments, wherein the concentration of

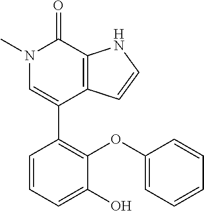 [SMILES: Cn1cc(-c2cccc(O)c2Oc2ccccc2)c2cc[nH]c2c1=O]

is about 0.002% to about 0.05%, about 0.0025% to about 0.01% or about 0.003% to about 0.008% by weight of the pharmaceutical composition.

The use of any one of the preceding embodiments, wherein the BET inhibitor is administered in an amount sufficient to reduce or reverse fibrotic remodeling in the lungs relative to bleomycin stimulated vehicle-treated subject.

The use of the preceding embodiment, wherein a hydroxyproline content is reduced by at least about 15%, at least about 20%, at least about 25%, at least about 30%, at least about 35%, at least about 40%, at least about 45%, at least about 50%, at least about 55%, or at least about 60%.

The use of any one of the preceding embodiments, wherein the hydroxyproline content is reduced by about 15% to about 60%.

The use of any one of the preceding embodiments, wherein the hydroxyproline content is reduced by about 20% to about 40%.

The use of any one of the preceding embodiments, wherein the hydroxyproline content is reduced by about 20%, about 30% or about 40%.

The use of any one of the preceding embodiments, wherein the hydroxyproline content is reduced by about 20% to about 25%.

The use of any one of the preceding embodiments, wherein the BET inhibitor is administered in an amount sufficient to reduce interstitial fibrosis and subacute interstitial inflammation relative to a bleomycin stimulated and vehicle-treated subject.

The use of any one of the preceding embodiments, wherein the BET inhibitor is administered in an amount sufficient to reduce interstitial fibrosis and subacute interstitial inflammation relative to a stimulated and vehicle-treated subject, wherein the interstitial fibrosis and subacute interstitial inflammation are reduced by at least about 10%, at least about 15%, at least about 20%, at least about 25%, or at least about 30%.

The use of any one of the preceding embodiments, wherein the amount of the BET inhibitor ranges from about 0.001% to about 0.1% by weight of a pharmaceutical composition.

The use of any one of the preceding embodiments, wherein the amount of the BET inhibitor is about 0.001% to about 0.06%, about 0.002% to about 0.05%, about 0.0025% to about 0.01%, or about 0.003% to about 0.008% by weight of the pharmaceutical composition.

The use of any one of the preceding embodiments, wherein the lowest pathology severity for interstitial fibrosis and subacute interstitial inflammation is observed in animals treated with BETi1 0.06 mg/mL.

The use of any one of the preceding embodiments, wherein there is a decrease in pathology severity for interstitial fibrosis and subacute interstitial inflammation in animals treated with BETi1.

The use of any one of the preceding embodiments, wherein there is a decrease in animals treated with BETi1 in the numbers of fibroblasts and collagen fibers in the lung interstitium.

The use of any one of the preceding embodiments, wherein upon topical application of BETi1 there is a decrease in infiltrates of lymphocytes, macrophages and neutrophils in the lung interstitium.

The method of any one of the preceding embodiments, wherein upon topical application of BETi1 there is a decrease in infiltrates of lymphocytes, macrophages and neutrophils in the lung interstitium.

The use of any one of the preceding embodiments, wherein the BETi compound is capable of significant downregulation of one or more (e.g., all) of IL17A/F, TGFβ, collagen and fibronectin or other genes involved in TGF-β-mediated myofibroblast differentiation.

The use of any one of the preceding embodiments, wherein there is improvement in the signs and symptoms of the fibrosis disorder or disease upon topical application of the compound.

The use of any one of the preceding embodiments, wherein the fibrosis disease or disorder is PF.

The use of any one of the preceding embodiments, wherein there is a reduction in one or more cytokine biomarkers or genes involved in fibrosis. In some embodiments, the one or more cytokine biomarkers or genes involved in fibrosis comprise or are chosen from IL17A/F, TGFβ, collagen and fibronectin genes or other genes involved in TGF-β-mediated myofibroblast differentiation. The use of any one of the preceding embodiments, wherein there is an increase in one or more cytokine biomarkers. In some embodiments, the one or more cytokine biomarkers comprise or are chosen from IL17A/F, and TGFβ.

The use of any one of the preceding embodiments, wherein the administration of

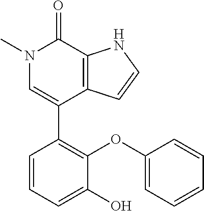 [SMILES: Cn1cc(-c2cccc(O)c2Oc2ccccc2)c2cc[nH]c2c1=O]

reduces fibrosis or scarring. In some embodiments,

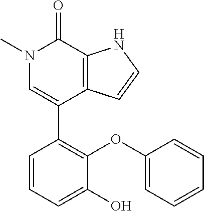 [SMILES: Cn1cc(-c2cccc(O)c2Oc2ccccc2)c2cc[nH]c2c1=O]

has a concentration of about 0.001% to about 1% by weight, or about 0.01% to about 1% by weight of the pharmaceutical composition or has a concentration of about 0.06 mg/mL to about 6 mg/mL, or about 0.06 mg/mL to about 3 mg/mL, or about 0.6 mg/mL to about 6 mg/mL, or about 0.6 mg/mL to about 3 mg/mL. In some embodiments, the concentration of

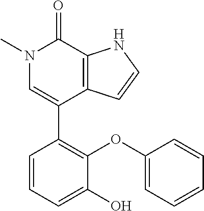 [SMILES: Cn1cc(-c2cccc(O)c2Oc2ccccc2)c2cc[nH]c2c1=O]

is about 0.1% by weight of the composition, or about 0.6 mg/mL. In some embodiments, the concentration of

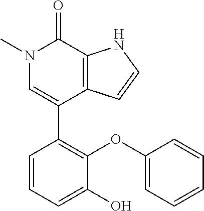 [SMILES: Cn1cc(-c2cccc(O)c2Oc2ccccc2)c2cc[nH]c2c1=O]

is about 1% by weight of the composition or about 3 mg/mL.

The use of any one of the preceding embodiments, wherein the BET inhibitor is administered in an amount sufficient to reduce TGF-β-mediated myofibroblast differentiation relative to bleomycin stimulated vehicle-treated mice.

The use of any one of the preceding embodiments, wherein the lung hydroxyproline content level is reduced by at least about 20% relative to bleomycin stimulated vehicle control after administration of BETi1.

The use of any one of the preceding embodiments, wherein the lung hydroxyproline content level is reduced by at least about 30% or at least by 35% relative to bleomycin stimulated vehicle control after administration of BETi1

The use of any one of the preceding embodiments, wherein the lung hydroxyproline content level is reduced by at least about 40% or at least about 50% relative to bleomycin stimulated vehicle control after administration of BETi1.

The use of the preceding embodiment, wherein the lung hydroxyproline content level is reduced by at least about 60%, or at least about 70%, or at least about 80%, or at least about 90%.

The use of the preceding embodiment, wherein the lung hydroxyproline content level is reduced by at least about 95%.

The use of any one of the preceding embodiments, wherein the BET inhibitor is administered in an amount sufficient to reduce numbers of fibroblasts and collagen fibers, relative to bleomycin stimulated vehicle-treated mice. The use of the preceding embodiment, wherein the BET inhibitor results in a reduction of numbers of fibroblasts and collagen fibers of at least about 25%, or at least about 30%, at least about 35%, or at least about 40%, or at least about 45%, or at least about 50% compared to bleomycin stimulated vehicle treated control. The use of the preceding embodiment, wherein the BET inhibitor result in a reduction of numbers of fibroblasts and collagen fibers secretion of 80% or less or of 70% or less or of 60% or less compared to bleomycin stimulated vehicle treated control.

The use of the preceding embodiment, wherein the amount of the BET inhibitor ranges from about 0.001% to about 1% by weight of a pharmaceutical composition. In some embodiments, the amount of the BET inhibitor ranges from about 0.001% to about 0.1% by weight of a pharmaceutical composition. In some embodiments, the amount of the BET inhibitor ranges from about 0.002% to about 0.08% by weight of a pharmaceutical composition. In some embodiments, the amount of the BET inhibitor ranges from about 0.1% to about 1% by weight of a pharmaceutical composition. In some embodiments, the amount of the BET inhibitor ranges from about 0.05% to about 5% or from about 0.075% to about 4%, or from about 0.1% to about 3%, or from about 0.125% to about 2.5%, or from about 0.15% to about 2% by weight of a pharmaceutical composition.

The use of any one of the preceding embodiments, wherein the administration of the BET inhibitor results in downregulation of fibrosis pathway. In some embodiments, the fibrosis pathway is downregulated by about >10%, >15%, >20%, >25%, >30%, >35%, >40%, or >45%, after administration of the BET inhibitor. In some embodiments, the fibrosis pathway is downregulated by about >50%, >60%, >70%, >75%, >80%, >85%, >90%, >95%, >96%, >97%, >98%, or >99% after administration of the BET inhibitor.

The use of any one of the preceding embodiments, wherein the administration of the BET inhibitor results in downregulation of TGFβ.

The use of any one of the preceding embodiments, wherein the administration of the BET inhibitor results in downregulation of one or more or all of IL17A/F, TGFβ, collagen and fibronectin or other genes involved in TGF-β-mediated myofibroblast differentiation. In some embodiments, the levels of one or more or all of IL17A/F, TGFβ, collagen and fibronectin or other genes involved in TGF-β-mediated myofibroblast differentiation are reduced by about >60%, >70%, >75%, >80%, >85%, >90%, >95%, >96%, >97%, >98%, or >99% after administration of the BET inhibitor. In some embodiments the levels of one or more of IL17A/F, TGFβ, collagen and fibronectin or other genes involved in TGF-β-mediated myofibroblast differentiation are reduced by about by about >10%, >15%, >20%, >25%, >30%, >35%, >40%, or >45%, after administration of the BET inhibitor.

In some embodiments, the pharmaceutical compositions may also be injected and may be prepared in the form of a solution, suspension or emulsion for such an application. Alternatively, the pharmaceutical compositions may be administered as nebulizer or a spray, including a nasal or buccal spray. Otherwise, the pharmaceutical compositions may be processed into a gel, cream, patch, implant or any other preparation for immediate and/or sustained release. Typically, the pharmaceutical compositions are processed into a gel, cream, lotion, foam or ointment for topical administration; or buccal spray for oral administration, or into a powder, solution or suspension (in a liquid vehicle) for inhalation delivery e.g., as a spray, mist, or puff.

In some embodiments, the compounds can be used in the treatment of diseases or conditions associated with the activity of Bromodomain and Extra-Terminal proteins. In some embodiments, there is provided a compound of the first aspect as specified, or a pharmaceutical composition of the second aspect for use, in the inhibition of Bromodomain and Extra-Terminal proteins.

Apart from or in addition to the bio markers discussed herein in relation to pigmentation disorders, particularly vitiligo, or fibrosis disorders, particularly PF, there are bio markers known to be implicated in inflammation. The potential to be effective against one or more disorders or diseases described herein may in one or more embodiments be illustrated by the ability of the BETi to modulate, reduce or, in some cases, upregulate biomarkers known or implicated for example in an inflammatory or immune response. Inflammatory pathways and various inflammatory biomarkers may, in some embodiments, without being bound by any theory be a precursor to or be, to some extent, involved or partially involved in pigmentation or fibrosis disorders. Inflammatory diseases rely on T helper cells Th1, Th2 and Th17 for innate and adaptive immunity responses, which affect either or both of the acute or chronic stages of the disease. Many cytokine and chemokines are upregulated in an inflammatory disease and the ability to reduce the levels of these inflammatory markers is evidence of the ability of a drug to ameliorate a disease. Such cytokine and chemokines include but are not limited to granulocyte-macrophage colony-stimulating factor (GM-CSF); interleukins IL-1, IL-2, IL-4, IL-6, IL-8, IL-13, IL-17, IL-22; chemokine (c-c motif) ligands CCL2, CCL27 and CCL20; tumour necrosis factor alpha (TNF-α); thymic stromal lymphopoietin (TSLP); and chemokine (c-x-c motif) ligand 9 (CXCL9). In some embodiments, members of soft pan BET inhibitor family are capable of inhibiting some or most markers of inflammation listed above.

In some embodiments the members of soft pan BETi family described herein (e.g. BETi1) have an affinity both to BDI and BDII but a higher (e.g., modest) selectivity to BDII. Thus, they may affect fewer markers of inflammation including, but not limited to, interleukins (IL-4, IL-13, IL-17 α, IL22, IL-31, IL-36γ); chemokine (c-c motif) ligand 2 (CCL2); and chemokine (c-x-c motif) ligand 10 (CXCL10), chemokine (c-c motif) ligand (CCL17), chemokine (c-c motif) ligand (CCL20), chemokine (c-c motif) ligand (CCL26), and Serpin B4. These important markers of disease predict efficacy for many inflammatory diseases, while selective BET BDII inhibitors focused profile suggests such compounds have an improved therapeutic index.

In some or more embodiments, there is provided a method of inhibiting Bromodomain and Extra-Terminal protein activity in a subject, said method comprising administering to a subject an effective amount of a BETi, or a pharmaceutical composition of BETi.

For BETi family members that are metabolically less stable in plasma and/or, e.g., have a liver clearance rate of less than 1 hour, in the event of transmembrane (e.g., transdermal, transmucosal or transparenchymal) delivery, following topical administration, systemic exposure is mitigated. This renders these ‘soft’ pan-BD BET suitable for topical application.

It has now been discovered that the BETIi disclosed herein targeting BRD4 have a binding affinity for BDI and BDII and a selectivity for BDII at lower concentrations. It has been found in accordance with the present disclosure that a specific set of BETIi have a broad activity which potentially makes them suitable for treatment of diseases or conditions associated with the activity of the Bromodomain and Extra-Terminal protein involved multiple, diverse inflammatory cell signaling pathways.

As BET proteins are key components of transcriptional processing for a wide range of proinflammatory and immunoregulatory genes, inactivation of BET proteins such as BRD4 may directly perturb the biosynthesis of pro-inflammatory proteins that are implicated in a wide variety of immune-inflammatory/fibrotic and myeloproliferative neoplastic disorders. For example, BET proteins act as epigenetic readers, that regulate the recruitment of transcriptional factors that are key to cytokine biosynthesis. Inhibiting BET proteins blocks transcriptional factor recruitment that prevents certain cytokine transcription.

It has been found in accordance with the present disclosure that imiquimod induced animals dosed topically with a composition comprising different concentrations of BETi1 resulted in reduction of the PASI score compared to placebo treated animals. The vehicle and the composition comprising BETi was well tolerated at two BETi1 concentrations tested (0.01% and 1%) by healthy mice. Animals treated with either vehicle or 0.1% BETi1 did not experience any determinable local skin reaction to treatment and there was no impact on fur re-growth. A dose dependent response was observed over the range of 0.001%-0.1% BETi1. The best PASI score was observed for the 0.1% dose. The PASI for the BETi1 1% dose was slightly lower than that observed for the 0.001% dose.

Animals treated with BETi 10.1% continued to gain body weight in a similar manner to healthy control group treated with vehicle. Clobetasol cream 0.05% group had a 17% reduction in body weight compared to the BETi1 0.1% group at treatment day 7. As mentioned below the steroid also resulted in skin thinning of the skin and this negative effect can be perceived to mask the scoring observed with the other parameters. Omitting it may on one level provide a clearer representation of the treatment potential of BETi since no marked weight loss or thinning is noted other than with the steroid. Also, without being bound by any theory it is suggested that the weight loss may be in part associated with systemic penetration and the concentration of the steroid.

Thus, a modified PASI (mPASI) value was calculated which is the same the PASI score but without including the scoring for induration (thickening of the skin). The modified PASI (mPASI) also referred herein as Composite Inflammation Severity Score is calculated in the same the manner as mean PASI score but excludes the scoring for induration (thickening of the skin). It is a composite mean score of erythema and peeling severity scored on a 4-point ordinal scale per domain (0=none, 1=mild, 2=moderate and 3=severe for a maximum score of 6.

Surprisingly, the BETi1 0.1% had comparable efficacy with respect to reduction in mPASI score to a commercial cream containing 0.05% clobetasol as a reference (Dermoval®) with an earlier onset seen for steroid. Moreover, there was a 94% reduction in mPASI for BETi1 0.1% relative to vehicle control group at Day 7. The mean percentage change is expressed from initiation of treatment phase. No appreciable improvement in clinical signs was seen for animals treated with placebo. In contrast, substantial resolution of clinical signs was observed in animals treated with BETi1 0.1%. Skin presented with normal physiology with no evidence of striae rubiae or atrophy and there was no evidence suggestive of intolerance. Substantial resolution of clinical signs was also observed in animals treated with a steroid. However, significant evidence of dermal atrophy (clear presence of both rhytides and deep wrinkles) and marked dermal translucency and elastolysis was also observed indicating weight loss and skin thinning.

All animals treated for during the inducation phase with 7 days of topically-applied imiquimod experienced a mean reduction in use of cage enrichment compared to pre-dose activity. Vehicle only treatment group had a marginal increase in use of cage enrichment compared to the induction phase. Both BETi1 1% and clobetasol 0.05% cream groups had recovered to pre-dose levels.

The pathogenesis of many autoimmune diseases is linked to the proliferation and activity of Th17 cells, which includes various diseases or disorders described herein. Measurements to evaluate the level of pro-inflammatory biomarkers (IL-1β, TNF-α, IL-18, IL-6, IL-8, IL-17, IL-23, and IL-36) in the animal model showed a strong correlation between improvement in clinical severity scores (mPASI) and reduction in many pro-inflammatory biomarkers relevant to Th17-mediated immune and/or autoimmune diseases. Dose-dependent reduction in biomarker expression was observed with BETi1 0.1% demonstrated as having the greatest effect. IL1(3 and IL-23 precipitate the polarization of naïve Th0 immune cells to Th17 cells. Th17 cells produce a range of cytokines that drive inflammation in autoimmune diseases. These include IL17, TL6, TL36 and TNFα.

There are number elevated inflammatory cytokines implicated in psoriasis which are also common in other inflammatory disorders such as Pyoderma Gangrenosum (PG), Palmar Plantar Pustulosis (PPP) and Generalized Pustular Psoriasis (GPP). Neutrophils play a key role in PG and IL-1, IL17, TNF-α, IL8, IL-6 and IL-23 are known to be upregulated in PG lesions. IL-1, IL-17 and IL-36 are the dominant cytokines increased in PPP. IL-1 and IL-36 are the dominant cytokines increased in GPP. Hence it is postulated that such soft pan-BD BET inhibitors which were shown to effective in reducing proinflammatory cytokine levels in psoriasis can potentially be used for treatment of rare, life-threatening/limiting neutrophilic dermatoses such as PG, PPP and GPP where there is significant unmet need due to a lack of indicated treatment options.

Without being bound by any theory, it was postulated that a similar expression pattern of these biomarkers to that of psoriasis would be consistent with the topical formulation comprising BETi being an effective treatment against PG, PPP and/or GPP. Moreover, a topically applied BETi (e.g. BETi1) may positively impact diseases involving multiple, diverse inflammatory cell signaling pathways that are active in rare neutrophilic dermatoses.

The present disclosure has evaluated this possible impact by using an ex-vivo cytokine release data from Th17 and Th2 stimulated human skin explants which mimic dermal inflammatory diseases. It has been surprisingly discovered that topical administration of BETi composition significantly reduced expression of several key pro-inflammatory proteins relevant to Th2 and Th17-mediated autoimmune diseases.

Human skin biopsy tissue was stimulated to induce a Th17 immuno-phenotype using a method derived from Feghali-Bostwick et al. Induction of a Th17 phenotype in human skin—a mimic of dermal inflammatory diseases, Methods and Protocols, 2, 45 (2019). In the study, the release of several key pro-inflammatory cytokines were substantially reduced when Th17-stimulated human skin tissue was treated with BETi1, resulting in greater than 95% inhibition of IL-17, IL-36 and CXCL10 release relative to vehicle control. BETi1 also demonstrated a superior anti-inflammatory effect on these cytokines when compared to the a janus kinase inhibitor (JAK1 and JAK2 inhibitor), Ruxolitinib, at identical concentrations.

LP-10 is an inflammatory cell chemoattractant secreted in response to interferon gamma. LP-10 is significantly overexpressed in many autoimmune diseases (>25-fold) verses healthy skin. A 95% inhibition of CXC motif chemokine ligand 10 (CXCL 10) expression, also known as LP-10, was demonstrated for skin treated with BETi1 as compared to vehicle skin and said inhibition was significantly better than a steroid (betamethasone) a janus kinase inhibitor (JAK1 and JAK2 inhibitor) (Ruxolitinib).

T-cells are polarized to Th1 and Th17 cells the latter of which drives the production of IL17A which further upregulates the migratory action of pro-inflammatory cells and further inflammatory cell activation. A 99.5% inhibition of interleukin 17-alpha (IL17A) expression was demonstrated for skin treated with BETi1 as compared to untreated skin and said inhibition was significantly better than treatment with a steroid or JAK inhibitor.

IL36γ is implicated in upregulating IL-17A signaling-related genes and so able to amplify keratinocyte inflammatory responses by promoting not only their own expression but also that of other cytokines related to Th17 signaling. A 95% inhibition of interleukin IL36γ expression was demonstrated for skin treated with BETi1 as compared to untreated skin and said inhibition was significantly better than treatment with a steroid or JAK inhibitor.

IL-4 is secreted by several inflammatory cells and drives differentiation of naïve Th0 cells to Th2 cells in adaptive immunity. Along with IL-13 and their respective receptors, IL-4 is a significant contributor to allergenic response in diseases such as atopic dermatitis, rhinitis and allergenic asthma. A 75% inhibition of interleukin IL-4 expression was demonstrated for Th2 stimulated human skin explants treated with BETi1 as compared to untreated skin and said inhibition was significantly better than treatment with a steroid or JAK inhibitor

Similar to IL-4, IL-13 is an important contributor to the pathogenesis of allergenic diseases. IL-13 is significantly over-expressed in atopic dermatitis lesions and in airway epithelial cells in asthma patients. A 93% inhibition of interleukin IL-13 expression was demonstrated for Th2 stimulated human skin explants treated with BETi1 as compared to untreated skin and said inhibition was significantly better than treatment JAK inhibitor and nearly comparable to treatment with a steroid.

IL-31 is overexpressed in several Th2-mediated inflammatory diseases. IL-31 has been implicated as having a direct role on pruritus signaling as the IL-31 receptor has been found in dorsal root ganglia that route transmission of sensory information to the spinal cord. A 95% inhibition of interleukin IL-31 expression was demonstrated for Th2 stimulated human skin explants treated with BETi1 as compared to untreated skin and said inhibition was significantly better than treatment with a JAK inhibitor and nearly comparable to treatment with a steroid.

In one or more embodiments, there is provided a BETi or a composition comprising the BETi (e.g. BETi1) for treatment or lessening the symptoms of diseases and disorders resulting from overexpression or downregulation of proinflammatory and immunoregulatory genes. In one or more embodiments, the BETi or compositions described herein can help reduce inflammation by reduction of certain cytokines involved in an inflammatory response. In one or more embodiments, certain cytokine levels are reduced by >50%, or >60%, or >70%, or >75%, or >80%, or >85%, or >90%, or >95%, or >96%, or >97%, or >98%, or >99% by treatment with BETi or topical BETi compositions. Preferably, treatment with compounds or topical compositions comprising BETi (e.g. BETi1) in one or more embodiments exhibit >50%, or >60%, or >70%, or >75%, or >80%, or >85%, or >90%, or >95%, or >96%, or >97%, or >98%, or >99% reduction in CXCL10 and inflammatory cytokines such as IL-17a, IL-36γ, IL-4, IL-13 or IL-31 levels relative to untreated Th-2 or Th-17 induced skin. Preferably, treatment with BETi (e.g. BETi1) or compositions comprising BETi in one or more embodiments exhibit >50% or >60%, or >70%, or >75% or >80%, or >85%, or >90%, reduction in inflammatory cytokines such as, IL-4, IL-13 and IL-31 levels. In one or more embodiments, treatment with BETi or topical compositions comprising BETi1 (e.g. BETi1) exhibit >90%, or >95%, or >96%, or >97%, or >98%, or >99% reduction in CXCL10 and inflammatory cytokines such as IL-17a, IL-36γ levels. As shown in Example 3, treatment with BETi1 exhibited >75% reduction in human skin induced to exhibit Th2 or Th17 phenotype, which is a mimic of dermal inflammatory diseases, in CXCL10, IL- and inflammatory cytokines such as 17a, IL-36γ, IL-4, IL-13 or IL-31 levels.

BETi or compositions comprising BETi in one or more embodiments exhibit 50%, or >60%, or >70%, or >75%, or >80%, or >85%, or >90%, or >95%, or >96%, or >97%, or >98%, or >99% reduction in CXCL9-11 and inflammatory cytokines such as IL-1β, IL-8, IL-17 α, TNF, IFNγ and IL-36 when compared to healthy skin.

In one or more embodiments, certain other cytokine levels are upregulated by about >50%, or >60%, or >70%, or >75%, or >80%, or >85%, or >90%, or >95%, or >96%, or >97%, or >98%, or >99% by treatment with BETi or topical BETi compositions. In some embodiments, certain cytokine levels are reduced by about >10%, or >15%, or >20%, or >25%, or >30%, or >35%, or >40%, or >45% by treatment with BETi or topical BETi compositions. In some embodiments certain other cytokine levels are upregulated by about >10%, or >15%, or >20%, or >25%, or >30%, or >35%, or >40%, or >45% by treatment with BETi or topical BETi compositions. In one or more embodiments, the WNT/β-catenin pathway is upregulated by about >50%, or >60%, or >70%, or >75%, or >80%, or >85%, or >90%, or >95%, or >96%, or >97%, or >98%, or >99% by treatment with BETi or topical BETi compositions. In some embodiments, WNT/β-catenin pathway include WNT16. In some embodiments, WNT16 and RAB3A are upregulated by about >50%, or >60%, or >70%, or >75%, or >80%, or >85%, or >90%, or >95%, or >96%, or >97%, or >98%, or >99% by treatment with BETi or topical BETi compositions. In some embodiments, SOX9, POMC, TL6, IL1a and b, TNF and EDN1 (vasoconstriction) are upregulated by about >50%, or >60%, or >70%, or >75%, or >80%, or >85%, or >90%, or >95%, or >96%, or >97%, or >98%, or >99% by treatment with BETi or topical BETi compositions.

In one or more embodiments, there is provided a compound composition comprising BETi for treatment or lessening the symptoms of diseases and disorders resulting from overexpression or downregulation of proinflammatory and immunoregulatory genes or for genes responsible for releasing cytokines selected from a group consisting of CD4+, CD8+, IL-17 α/F, IL-23, TNFα, IL-1β, TL-6, IL-18, TGFβ, TL-4, IL-5, IL-13, IL-31 and TL-15. In some embodiments, and without being bound by theory, the treatment BETi or topical BETi compositions 1% prevents detachment and subsequent loss of melanocytes from the basal layer implying that E-cadherin adhesion is still functional. In some embodiments, and without being bound by theory, the treatment may be effective in reducing, in preventing or in arresting to some extent the melanocytorhagy process, or in maintaining the location of melanocytes within the basal layer of the skin epidermis, or in restoring to some extent melanocytes, and on the surprising downregulation of cytokines SOX9, POMC IL6, IL1a and b, TNF and EDN1 (vasoconstriction) and upregulation of the WNT/β-catenin pathway (e.g., WNT16) and RAB3A genes. In one or more embodiments, there is provided a composition to treat or lessen the symptoms of resulting from inactivation of BET proteins such as BRD4. In one or more embodiments, there is provided a composition to treat or lessen the symptoms of directly perturbed biosynthesis of pro-inflammatory proteins that are implicated in a wide variety of immune-inflammatory/fibrotic and myeloproliferative neoplastic disorders. In one or more embodiments, a topically applied BETi positively impacts diseases involving multiple, diverse inflammatory cell signaling pathways that are active in rare neutrophilic dermatoses. In one or more embodiments, a topically applied BETi is used as a convenient, non-biologic treatment option for both acute control and chronic management of immuno-inflammatory indications, wherein the damaging effects of unrestricted inflammatory signaling activity is common. In one or more embodiments, a topically applied BETi is used for a wide variety of diseases. In one or more embodiments, a topically applied BETi is used for effectively treating pigmentation disorders e.g., vitiligo. In some embodiments, a topically applied BETi is used for effectively treating fibrosis disorders e.g., PF. In one or more embodiments, a topically applied BETi is used for effectively treating immuno-inflammatory diseases. In one or more embodiments, a topically applied BETi is used to treat rare diseases and/or inflammation related conditions or disorders. In some embodiments, the composition is applied topically including through inhalation, in some orally and in some both topically and orally. In one or more embodiments, the composition comprises a carrier and one or more active pharmaceutical ingredients (active agents). In some embodiments, the active agent comprises a BET inhibitor. In some embodiments, the active agent comprises a pan BET inhibitor. In some embodiments, the active agent comprises a selective BET inhibitor. In some embodiments the active agent is a combination of a pan BET inhibitor (e.g., a BETi) and a selective BET inhibitor.

In one or more embodiments, the carrier is oil-based. In one or more embodiments, the carrier is petrolatum-based. In some embodiments, the carrier is water-based. In some embodiments, the carrier is hydrophilic-based. In some embodiments, the carrier is liposome-based. In some embodiments, the carrier is particle- or powder-based. In some embodiments, the carrier is micelle-based. In some embodiments, the carrier is emulsion-based. In some embodiments, the composition is a suspension of the BETi. In some embodiments, the composition is a solution of the BETi. In some embodiments, the composition is in part a suspension and is in part a solution of the BETi compound.

The present application provides methods of treating a skin disorder comprising applying a composition comprising a BETi (e.g., BETi1) to the skin of a subject. In one or more embodiments, administering a topical composition comprising BETi (e.g., BETi1) is designed to address the multi-factorial nature PG which includes the inflammatory factor and wound healing factor while avoiding scarring. In one or more embodiments a method of treatment or prophylaxis of a disorder or disease in a patient in need thereof, wherein the method comprises topically administering a therapeutically effective amount of a BETi (e.g., BETi1) and wherein the disorder or disease is chosen from a group consisting of wounds, cuts, PG, GPP, PPP, hidradenitis suppurativa, diabetic foot ulcers, chronic wounds, and pulmonary and fibrosis. The present application provides methods of treating a pigmentation skin disorder, e.g., vitiligo comprising applying a composition comprising a BETi (e.g., BETi1) to the skin of a subject.

In one or more embodiments, administering a topical BETi (e.g., BETi1) composition can be used for treatment of a disease by targeting CD4+ Th17 and wherein the key immune targets implicated in the disease are IL17α/F, IL23, TNFα. In one or more embodiments, administering a topical BETi (e.g., BETi1) composition can be used for treatment of a disease by targeting complex gene expression markers resulting in the downregulation of cytokines SOX9, POMC, IL6, IL1a and b, TNF and EDN1 (vasoconstriction) and upregulation of the WNT β-catenin pathway (e.g., WNT16) and RAB3A.

In one or more embodiments, administering a topical BETi (e.g. BETi1) composition can be used for treatment of disease by targeting CD4+ Th2 and wherein the key immune targets implicated in the disease are IL4, IL 5, 13 and IL 31.

In one or more embodiments, administering a topical BETi (e.g. BETi1) composition can be used for treatment of a disease by targeting CD8+Th17 and wherein the key immune targets implicated in the disease are IL17A/F, IL23, TNFα.

In one or more embodiments, administering a topical BETi (e.g. BETi1) composition can be used for treatment of hidradenitis suppurativa by targeting CD4+ Th17 and wherein the key immune targets are IL13, IL6, IL18, TNFα.

In one or more embodiments, administering a topical BETi (e.g. BETi1) composition can be used for treatment of a disease by targeting CD8+Th17 and wherein the key immune targets implicated in the disease are IL-1β and IL-15.

In one or more embodiments, administering a topical BETi (e.g. BETi1) composition can be used for treatment of diabetic foot ulcers.

In one or more embodiments, administering a topical BETi (e.g. BETi1) composition can be used for treatment of chronic wounds.

In one or more embodiments, administering a topical BETi (e.g. BETi1) composition can be used for treatment of disease by targeting CD4+ Th2 and wherein the key immune targets implicated in the disease are IL-4, IL-5 and IL-3.

In one or more embodiments, administering a topical BETi (e.g. BETi1) composition can be used for treatment of a disease by targeting CD4+ Th17 and wherein the key immune targets implicated in the disease are IL17A/F, TGFβ.

Various therapies are used to try and treat vitiligo. Camouflage therapy, including use to minimize tanning, thereby limiting the contrast between affected and normal skin, makeups, hair dyes if vitiligo affects the hair. Depigmentation therapy e.g., with monobenzone can be used if the disease is extensive and is applied to pigmented patches of skin to turn them white to match the areas of vitiligo. Repigmentation therapy, including corticosteroids can be taken orally (as a pill) or topically (as a cream put on the skin). Side effects can include skin thinning or striae (stretch marks), topical vitamin D analogs and topical immunomodulators such as calcineurin inhibitors. Light therapy, including narrow band ultraviolet B (NB-UVB), excimer laser treatment delivered to small, targeted areas emits a wavelength of ultraviolet light close to that of narrow band UVB, and combining oral psoralen and UVA (PUVA) is used to treat large areas of skin with vitiligo. In some cases surgery may be an option, including grafts and cellular transplants.

The present application provides methods of treating a fibrosis disorder, e.g., PF comprising applying a composition comprising a BETi (e.g., BETi1) to the respiratory system of a subject.

In some embodiments, administering a topical BETi (e.g. BETi1) composition can be used for treatment of a disease by targeting complex gene expression markers resulting in the downregulation of e.g., cytokines IL17A/F, TGFβ, collagen and fibronectin or other genes involved in TGF-β-mediated myofibroblast differentiation.

Treatment options for PF are very limited. Though research trials are ongoing, there is no evidence that any medications can significantly help this condition. Lung transplantation is the only therapeutic option available in severe cases. Since some types of lung fibrosis can respond to corticosteroids (such as prednisone) and/or other medications that suppress the body's immune system, these types of drugs are sometimes prescribed in an attempt to slow the processes that lead to fibrosis and decrease lung inflammation and subsequent scarring, but, only a minority of patients respond to corticosteroids alone, so additional immunosuppressants and alternative treatments are needed. Lung transplantation is the only therapeutic option available in severe cases.

Clobetasol propionate has a different mechanism of action from a BETi. In one or more embodiments, the topical BETi compositions avoid one or more untoward systemic and skin related side effects that can occur with oral administration. In one or more embodiments, treatment with a topical BETi composition can avoid one or more systemic and skin-related side effects associated with steroids and other treatments. For example, common skin related side effects of clobetasol propionate include burning, pruritus, erythema, pain, irritation, rash, peeling, urticaria, and contact dermatitis. Prolonged use of topical corticosteroids is thought to increase the risk of possible side effects, and high-strength steroids may be absorbed into the body. Their immunosuppressive action can also lead to secondary skin infections. For example, clobetasol propionate is a highly potent topical corticosteroid that has been shown to suppress the HPA axis at doses as low as 2 g per day. Systemic absorption of topical corticosteroids has resulted in reversible HPA axis suppression, manifestations of Cushing's syndrome, hyperglycemia, and glucosuria in some patients. Conditions that augment systemic absorption include the application of more potent corticosteroids, use over large surface areas, prolonged use, and the addition of occlusive dressings. Therefore, patients receiving a large dose of a potent topical steroid applied to a large surface area are evaluated periodically for evidence of HPA axis suppression. Use of Dermoval 0.05% in pediatric patients under 12 years of age is not recommended. Pediatric patients may demonstrate greater susceptibility to topical corticosteroid-induced HPA axis suppression and Cushing's syndrome than mature patients because of a larger skin surface area to body weight ratio.

The topical BETi composition described herein can in one or more embodiments avoid undesirable side effects such as skin thinning and change in body weight. In one or more embodiments, the BETi topical compositions herein can provide an effective, safe, well tolerated long term solutions to treat skin disorders unlike steroids which are preferably limited for use for short periods and are not well tolerated and can have undesirable side effects as indicated herein.

In some embodiments, the topical BETi compositions avoid one or more untoward systemic and related side effects that can occur with oral administration. In some embodiments, treatment with a topical BETi composition can avoid one or more systemic and -related side effects associated with steroids and other treatments. For example, common side effects of prednisone include acne, blurred vision, changes in behavior or mood, dizziness, elevated blood pressure levels, elevated blood sugar levels, fluid retention, headache, increased appetite, insomnia or fatigue, nausea, restlessness and inability to stay still (akathisia), sleep problems, swelling, thinning skin, vomiting, weight gain. Prolonged use of topical corticosteroids is thought to increase the risk of possible side effects, and high-strength steroids may be absorbed into the body. Their immunosuppressive action can also lead to secondary skin infections.

The topical BETi compositions described herein can in some embodiments avoid or ameliorate undesirable side effects such as membrane (e.g., skin) thinning and change in body weight. In some embodiments, the BETi topical compositions herein can provide an effective, safe, well tolerated long term solutions to treat fibrosis disorders unlike steroids which are preferably limited for use for short periods and are not well tolerated and can have undesirable side effects as indicated herein.

In one or more embodiments, treatment with commercial steroid product does not show good tolerability, as evidenced herein by loss of body weight and skin thinning in psoriasis animal model treated with Dermoval® cream containing 0.05% clobetasol. The topical BETi composition described herein can in one or more embodiments avoid such undesirable side effects. In one or more embodiments, the BETi topical compositions herein can provide an effective, safe, well tolerated long term solutions to treat skin disorders unlike steroids which are preferably limited for use for short periods and are not well tolerated and can have undesirable side effects as indicated herein.

In one or more embodiments, there is provided a topical composition comprising a BETi1 or a pharmaceutically acceptable salt thereof and a carrier in which BETi is suspended to treat or lessen the symptoms of IL17 related conditions, such as PG, GPP, psoriasis, and PPP. In one or more embodiments, there is provided a topical composition comprising a BETi or a pharmaceutically acceptable salt thereof and a carrier in which BETi is suspended to treat or lessen the symptoms of PG wherein the key immune targets are IL-1β, IL-17, IL-8, TNF, IFNγ and IL-36γ

In one or more embodiments, there is provided a topical composition comprising a BETi1 or a pharmaceutically acceptable salt thereof and a carrier in which BETi1 is suspended to treat or lessen the symptoms of IL17 related conditions, such as PPP and wherein the key immune targets are IL-36γ, IL-17, IL-8 and IL-1.

In one or more embodiments, there is provided a topical composition comprising a BETi1 or a pharmaceutically acceptable salt thereof and a carrier in which BETi1 is suspended to treat or lessen the symptoms of IL17 related conditions, such as GPP and wherein the key immune targets are IL-36γ, IL-17, IL-8 and IL-1β.

In one or more embodiments, there is provided a topical composition comprising a BETi1 or a pharmaceutically acceptable salt thereof and a carrier in which BETi is suspended to treat or lessen the symptoms of pigmentation related conditions, such as vitiligo.

In some embodiments, there is provided a topical composition comprising a BETi1 or a pharmaceutically acceptable salt thereof and a carrier in which BETi is suspended to treat or lessen the symptoms of fibrosis related conditions, such as PF.

In one or more embodiments, there is provided a topical composition comprising a BETi (e.g., BETi1) or a pharmaceutically acceptable salt thereof and a carrier in which BETi is suspended to treat or lessen the symptoms of joint or joint relted related conditions, such as an arthritis.

In some embodiments the introduction of surfactants in formulations comprising BETi may further help the treatment. In some embodiments the introduction of emollients in formulations comprising BETi may further help the treatment.

In one or more embodiments a BETi, may be effective in treating dermatological disorders involving inflammation and/or lymphocyte action. In some embodiments, the dermatological disorder can include one or more of, a dermatomyositis, eczema, and scarring.

Skin wound healing is a process that generally consists of three sequential phases: inflammation, proliferation, and regeneration. During the activation of the inflammatory cascade numerous inflammatory cells infiltrate the damaged area and release cytokines. The cytokines stimulate the migration of keratinocytes and fibroblasts to the wound site and subsequent proliferation of these cells begins (e.g., 4-5 days) later. Fibroblasts secrete extracellular matrix (ECM) proteins such as fibronectin, collagen and hyaluronic acid, resulting in the formation of granulation tissue. During the proliferation phase, abundant vascularization and angiogenesis play a key role in supplying the inflammatory cells and fibroblasts for the formation of an occasional granulation matrix.

Dehydration causes cytokine-mediated signaling from keratinocytes to dermal fibroblasts which increase production of collagen. However, over production of collagen can result in thick raised unsightly scars. Occlusion keeps the scar hydrated, so fewer signals are sent to the wound creating less scar tissue.

It has been surprisingly found that treatment of skin wounds with a formulation comprising 1.0% BETi1, rapidly reduced the swelling at wound edges and said reduction was significantly different on Days 15 16 and 17 compared to placebo and hydroalcoholic gel. In some embodiments, reduction of swelling resulted in reduced presence of tissue mass under the scar. Without being bound by any theory, reduction in swelling may be attributed to the anti-inflammatory effect of BETi1. Surprisingly, treatment with a topical formulation comprising 1.0% BETi1 had a significant effect on the global internal score as assessed by macroscopic internal scoring compared to placebo (P<0.01) and hydroalcoholic gel (P<0.05). The global internal score is the sum of visibility of scars score, presence of tissue mass under the scar and vascularization. Animals treated with a formulation comprising 1.0% BETi1 had improved internal scar visibility when compared to either vehicle or hydroalcoholic gel. Formulation comprising 1.0% BETi1 had significant effect on the visibility of scars score compared to placebo and hydroalcoholic gel (P<0.005). Correspondingly, animals treated with formulation comprising 1.0% BETi1 had less tissue mass/fibrosis under the scar compared to either vehicle or hydroalcoholic gel (P<0.05). Animals treated with a formulation comprising 1.0% BETi1 had numerically higher degree of wound vascularization when compared to either vehicle or hydroalcoholic gel (not statistically significant) compared to placebo and hydroalcoholic gel. Overall, excisions treated formulation comprising 1.0% BETi1 had a lower global internal score compared to either vehicle or hydroalcoholic gel indicative of an improved scar outcome. Generally, scars of mice treated with formulation comprising 1.0% BETi appeared more aesthetic, flatter and less visible which could be due to a faster re-modelling of skin tissue. They exhibited little evidence of residual swelling. Wounds treated with BETi1 formulation appeared more macular in nature compared to vehicle or the hydroalcoholic gel. Incision sites appear less distinct and left a more aesthetic outcome compared to other treatments. In wounds treated with vehicle there was still evidence of minor swelling around incision sites. Wounds treated with hydroalcoholic gel moderate swelling was clearly evident at end of treatment. Although healed, residual scabbing still remained and incision sites were clearly visible. Without being bound by any theory it is postulated that wound healing properties of the BETi1 formulation may improve healing of wounds and particularly challenging wounds for example as seen with PG with little or no scarring.

Despite its occlusive properties, silicone provides optimal permeability to maximize oxygen transfer across the surface of the skin which enhances wound healing. Silicone also helps transfer tension from the edges of the wound to the silicone. These tension forces that normally widen scars are reduced due to absorption of tension by the silicone. Silicone also reduces the redness of the scar by preventing the creation of new blood vessels. Silicone also creates a negative static field which aligns and organizes the collagen fibers in a more uniform pattern. The negative field also tends to pull in or cause the involution of raised scars. In summary occlusion potential resulting in less production of collagen, tension transfer, decreased capillary activity and collagen alignment from silicone treatment may result in enhanced scar healing. Silicone can, in one or more embodiments, be a suitable carrier for the BETi compounds of the present invention.

Silicone-based formulations comprising BETi may provide an alternative lesser occlusive or non-occlusive platform for the treatment and reduction of scars. In one or more embodiments the silicone-based formulations comprising BETi can be applied to improve the appearance of scars and to prevents abnormal or excessive scar formation. In one or more embodiments silicone-based formulations comprising BETi can diminish the appearance of hypertrophic scars and keloids. In some embodiments, silicone-based formulations comprising BETi can diminish scars and keloids with a raised and/or discolored appearance. In some embodiments, silicone-based formulations comprising BETi can soften and flatten raised scars. In some embodiments, silicone-based formulations comprising BETi can reduce the redness associated with scars. In some embodiments, silicone-based formulations comprising BETi can be effective for both old and new scars. Silicone-based formulations comprising BETi may provide an alternative lesser occlusive or non-occlusive platform for the treatment and reduction of pigmentation disorders, such as vitiligo. In one or more embodiments the silicone-based formulations comprising BETi can be applied to improve the appearance of vitiligo, maintain pigmentation (retention of melanocytes and/or melanin at the basal layer) and to prevent or reduce pigmentation (melanocytes and/or melanin) loss. In one or more embodiments silicone-based formulations comprising BETi are suitable for use in adults, teenagers, adolescents, and children. In some embodiments, silicone-based formulations comprising BETi can be suitable for use on people with sensitive skin. In some embodiments silicone-based formulations comprising BETi can be used on scars that result from PG, surgery, injury, burns, acne, rosacea, psoriasis, dermatitis, cuts, insect bites, and others.

In one or more embodiments, the introduction of emollients in formulations comprising BETi may further help the treatment and reduction of scars. In some embodiments, they may also help reduce itch. In some embodiments they may help unclog skin pores. Unblocking the pilosebaceous units may be beneficial and especially in the case of facial scars. In one or more embodiments, beneficial emollients comprise an isopropyl ester, e.g., isopropyl isostearate and/or a saturated or branched hydrocarbon oil e.g., squalane. In one or more embodiments, beneficial emollients comprise one or more of an MCT oil, mineral oil, or IPP. In one or more embodiments, beneficial emollients comprise a plant-based oil such as soybean oil or coconut oil. Such oils may have antibacterial properties. In one or more embodiments, upon application, the gel is to form an adhesive film over the area of the scar.

In one or more embodiments the composition comprises an essential oil. Essential oils are plant oils extracted by distillation. Nonlimiting examples of essential oils include agar oil, ajwain oil, angelica root oil, anise oil, balsam oil, basil oil, bergamot oil, black Pepper essential oil, buchu oil, cannabis flower essential oil, caraway oil, cardamom seed oil, carrot seed oil, cedarwood oil, chamomile oil, cinnamon oil, cistus, citronella oil, clary Sage, clove leaf oil, coriander, costmary oil, cranberry seed oil, cumin oil/Black seed oil, cypress, davana oil, dill oil, eucalyptus oil, fennel seed oil, fenugreek oil, frankincense oil, galbanum, geranium oil, ginger oil, grapefruit oil, henna oil, jasmine oil, juniper berry oil, lavender oil, lemon oil, lemongrass oil, litsea cubeba oil, melissa oil (Lemon balm), mentha arvensis oil/mint oil, mugwort oil, mustard oil, myrrh oil, neroli oil, orange oil, oregano oil, orris oil, parsley oil, patchouli oil, perilla essential oil, pennyroyal oil, peppermint oil, pine oil, rose oil, rosehip oil, rosemary oil, rosewood oil, sassafras oil, savory oil, schisandra oil, spearmint oil, star anise oil, tarragon oil, tea tree oil, thyme oil, vetiver oil, yarrow oil and ylang-ylang oil. Many essential oils are used as medical and aromatherapy applications, and thus, can be considered as therapeutically active hydrophobic solvents.

In one or more embodiments, compositions that provide the scar treatment potential of low-occlusive silicone-based BETi that can modulate the inflammatory response to improve the treatment, reduction and healing of scars may be beneficial.

In one or more embodiments compositions that provide the skin treatment potential of low-occlusive silicone-based BETi that can modulate the inflammatory response to improve the treatment, reduction and healing of vitiligo may be beneficial.

In one or more embodiments, the effect of administering a composition comprising a BETi is achieved by delivering the BETi onto and into the skin or mucosa or follicles or body cavity or mucosal surface or parenchyma (e.g., within the lung, for example, by inhalation) or lung tissue. In one or more embodiments, systemic penetration through the skin, mucosa or follicles or body cavity or mucosal surface or tissue is low. In one or more embodiments, systemic penetration through the skin, mucosa or follicles or body cavity or mucosal surface or parenchyma or lung tissue is less than about 20%, is less than about 15%, is less than about 10%, less than about 9%, less than about 8%, less than about 7%, less than about 6%, less than about 5%, less than about 4%, less than about 3%, less than about 2%, less than about 1.8%, less than about 1.7%, less than about 1.6%, less than about 1.5%, less than about 1.4%, less than about 1.3%, less than about 1.2%, less than about 1.1%, less than about 1%, less than about 0.8%, less than about 0.6%, less than about 0.5%, less than about 0.4%, or less than about 0.1% of the BETi applied to the skin or body cavity or mucosal surface or parenchyma or lung tissue. In one or more embodiments, systemic delivery or systemic penetration through the skin, mucosa or follicles or body cavity or mucosal surface or parenchyma or lung tissue can supplement the effects produced by non-systemic delivery onto and into the skin, mucosa, or follicles or body cavity or mucosal surface or parenchyma or lung tissue.

The use of any one of the preceding embodiments, wherein cartilage is not impacted negatively. The use of any one of the preceding embodiments, wherein application of a BETi is tolerated by cartilage. The use of any one of the preceding embodiments, wherein application of a BETi does not result in cartilage damage or material cartilage damage. The use of any one of the preceding embodiments, wherein application of a BETi can result in some cartilage repair.

The pharmaceutical composition of any one of the preceding embodiments, wherein cartilage is not impacted negatively. The pharmaceutical composition of any one of the preceding embodiments, wherein application of a BETi is tolerated by cartilage. The pharmaceutical composition of any one of the preceding embodiments, wherein application of a BETi does not result in cartilage damage or material cartilage damage. The pharmaceutical composition of any one of the preceding embodiments, wherein application of a BETi can result in some cartilage repair.

The use of any one of the preceding embodiments, wherein unwanted side effects or adverse reactions of corticosteroid injections or infusion are avoided.

The pharmaceutical composition of any one of the preceding embodiments, wherein one or more unwanted side effects or adverse reactions of corticosteroids are avoided.

The use of any one of the preceding embodiments, wherein one or more unwanted side effects or adverse reactions of triamcinolone injections or infusion are avoided.

The pharmaceutical composition of any one of the preceding embodiments, wherein one or more unwanted side effects or adverse reactions of triamcinolone injections or infusion are avoided.

The use of any one of the preceding embodiments, wherein unwanted adverse reactions of triamcinolone injections include, hypersensitivity reactions, such as anaphylaxis, joint infection and damage, increased risk of infections, alterations in endocrine function, cardiovascular and renal effects, increased intraocular pressure, gastrointestinal perforation, alternations in bone density and behavioral and mood disturbances.

The pharmaceutical composition of any one of the preceding embodiments, wherein unwanted adverse reactions of triamcinolone injections include, hypersensitivity reactions, such as anaphylaxis, joint infection and damage, increased risk of infections, alterations in endocrine function, cardiovascular and renal effects, increased intraocular pressure, gastrointestinal perforation, alternations in bone density and behavioral and mood disturbances.

The use of any one of the preceding embodiments, wherein one or more unwanted side effects or adverse reactions of dexamethasone injections or infusion are avoided.

The pharmaceutical composition of any one of the preceding embodiments, wherein one or more unwanted side effects or adverse reactions of dexamethasone are avoided.

The use of any one of the preceding embodiments, wherein unwanted adverse reactions of dexamethasone include, fluid and electrolyte disturbances, musculoskeletal, gastrointestinal, neurologic, dermatologic, endocrine, ophthalmic, metabolic cardiovascular, anaphylactoid or hypersensitivity reactions, thromboembolism, weight gain, increased appetite, and nausea.

The pharmaceutical composition of any one of the preceding embodiments, wherein unwanted adverse reactions of dexamethasone include, fluid and electrolyte disturbances, musculoskeletal, gastrointestinal, neurologic, dermatologic, endocrine, ophthalmic, metabolic cardiovascular, anaphylactoid or hypersensitivity reactions, thromboembolism, weight gain, increased appetite, and nausea.

The use of any one of the preceding embodiments, wherein there is an improvement in at least one symptom of the joint disorder or disease upon intra-arterial or intra-venous application of about 0.05% to about 15%, such as about 0.01% to about 10%, about 0.1% to about 10%, or about 1% to about 10% of the BETi.

The use of any one of the preceding embodiments, wherein there is an improvement in at least one symptom of the joint disorder or disease upon local application within the joint of about 0.1% to about 15% of the BETi or in such other amounts as described herein, such as about 0.05% to about 15%, such as about 0.01% to about 10%, about 0.1% to about 10%, or about 1% to about 10%.

The use of any one of the preceding embodiments, wherein the joint disease or disorder is rheumatoid arthritis.

The pharmaceutical composition of any one of the preceding embodiments, wherein the composition comprises a solution of the compound.

The pharmaceutical composition of any one of the preceding embodiments, wherein the composition comprises a suspension of the compound.

The pharmaceutical composition of any one of the preceding embodiments, wherein the compound is micronized.

The pharmaceutical composition of any one of the preceding embodiments, wherein the compound comprises nano particles.

The pharmaceutical composition of any one of the preceding embodiments, wherein the compound is encapsulated.

The pharmaceutical composition of any one of the preceding embodiments, wherein the compound is a sustained or extended-release dosage form.

The pharmaceutical composition of any one of the preceding embodiments, wherein the sustained or extended-release dosage form also comprises an immediate release component.

The pharmaceutical composition of any one of the preceding embodiments, wherein the immediate release component is in a form different from the sustained or extended-release component.

The pharmaceutical composition of any one of the preceding embodiments, wherein the immediate release component comprises an amount of solubilized compound.

The pharmaceutical composition of any one of the preceding embodiments, wherein the sustained or extended-release component and initial release component provides an initial therapeutic effect following administration.

The pharmaceutical composition of any one of the preceding embodiments, wherein the sustained or extended-release component provides a therapeutic effect following the initial releases subsidence.

The pharmaceutical composition of any one of the preceding embodiments, wherein the sustained or extended-release dosage form provides steady state release for a period of about 2, about 3, about 4, about 5, about 6, about 7, about 8, about 9, about 10, about 11, or about 12 weeks.

The pharmaceutical composition of any one of the preceding embodiments, wherein the sustained or extended-release dosage form provides a sustained efficacy even though the compound is no longer resident at the site of administration.

The pharmaceutical composition of any one of the preceding embodiments, wherein the sustained or extended-release dosage form provides efficacy for a period after the compound is no longer released in a therapeutic effective amount.

The use of any one of the preceding embodiments, wherein there is a dose-dependent reduction in inflammation.

The use of any one of the preceding embodiments, wherein the dose-dependent reduction in inflammation is a reduction in thickness or girth of a joint or limb.

The use of any one of the preceding embodiments, wherein the dose-dependent reduction in inflammation is a reduction in thickness or girth of foot, hand, digit, elbow or knee.

The use of any one of the preceding embodiments, wherein the joint in which thickening is inhibited is a joint in a foot, hand, digit, elbow, or knee. In some embodiments, the joint is a knee or elbow joint.

The use of any one of the preceding embodiments, wherein the higher doses (e.g., 1 mg/kg and 10 mg/kg) treatment groups demonstrated a marked inhibition and the strongest inhibition of joint thickening when compared to untreated joint or limb e.g., foot.

The use of any one of the preceding embodiments, wherein lower doses (0.01 and 0.1 mg/kg) of

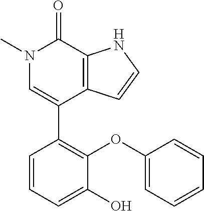 [SMILES: Cn1cc(-c2cccc(O)c2Oc2ccccc2)c2cc[nH]c2c1=O]

or vehicle had no appreciable impact in preventing thickening.

The use of any one of the preceding embodiments, wherein inhibition of thickening is about 10%, about 20%, about 30%, about 40%, about 50% about 60%, about 70%, or about 80%.

The use of any one of the preceding embodiments, wherein the higher doses of

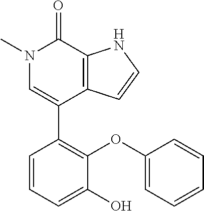 [SMILES: Cn1cc(-c2cccc(O)c2Oc2ccccc2)c2cc[nH]c2c1=O]

(e.g., 1 mg/kg and 10 mg/kg) treatment groups were superior to dexamethasone IA 1 mg/kg and in line with 1 mg/kg dexamethasone IP systemic dose.

The use of any one of the preceding embodiments, wherein the higher doses (e.g., 1 mg/kg and 10 mg/kg) treatment groups were in line with 1 mg/kg dexamethasone IP systemic dose.

The use of any one of the preceding embodiments, wherein there is a dose-dependent reduction in arthritic scoring or severity.

The use of any one of the preceding embodiments, wherein the dose-dependent reduction in arthritic scoring or severity is a reduction in:
(a) definite redness and swelling of the ankle/wrist or apparent redness and swelling limited to individual digits, regardless of the number of affected digits;
(b) severe redness and swelling of the ankle/wrist;
(c) redness and swelling of the entire foot/paw including digits; and/or
(d) maximally inflamed limb with involvement of multiple joints. The use of any one of the preceding embodiments, wherein the higher doses (e.g., 1 mg/kg and 10 mg/kg) treatment groups demonstrated a marked effect and the strongest reduction of arthritic scoring or severity when compared to untreated joint or limb e.g., foot.

The use of any one of the preceding embodiments, wherein lower doses (0.01 & 0.1 mg/kg) of

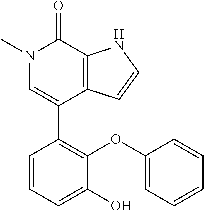 [SMILES: Cn1cc(-c2cccc(O)c2Oc2ccccc2)c2cc[nH]c2c1=O]

or vehicle had no appreciable impact in arthritic scoring or severity.

The use of any one of the preceding embodiments, wherein the reduction in arthritic scoring or severity is about 1, about 2, about 3, and about 4.

The use of any one of the preceding embodiments, wherein, the higher doses of

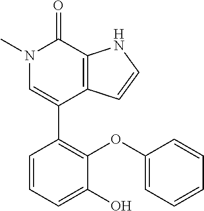 [SMILES: Cn1cc(-c2cccc(O)c2Oc2ccccc2)c2cc[nH]c2c1=O]

(e.g., 1 mg/kg and 10 mg/kg) treatment groups were superior to dexamethasone IA 1 mg/kg.

The use of any one of the preceding embodiments, wherein weight loss plateaus at about study day 5/6 and the reverses for all treated groups (weight gain).

The use of any one of the preceding embodiments, wherein subjects treated with systemic dexamethasone failed to experience meaningful weight gain.

The use of any one of the preceding embodiments, wherein the administration of

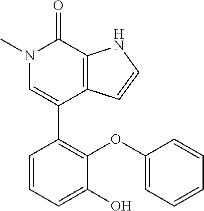 [SMILES: Cn1cc(-c2cccc(O)c2Oc2ccccc2)c2cc[nH]c2c1=O]

reduces rheumatoid arthritis.

The use of any one of the preceding embodiments, wherein

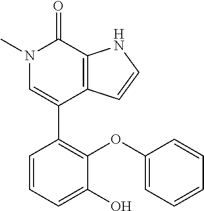 [SMILES: Cn1cc(-c2cccc(O)c2Oc2ccccc2)c2cc[nH]c2c1=O]

has a concentration of about 0.1% to about 15% by weight of the pharmaceutical composition or in such other amounts as described herein.

The use of any one of the preceding embodiments, wherein the concentration of

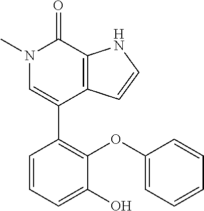 [SMILES: Cn1cc(-c2cccc(O)c2Oc2ccccc2)c2cc[nH]c2c1=O]

is about 1% by weight of the composition.

The use of any one of the preceding embodiments, wherein the concentration of

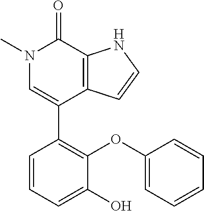 [SMILES: Cn1cc(-c2cccc(O)c2Oc2ccccc2)c2cc[nH]c2c1=O]

is about 5% by weight of the composition.

The use of any one of the preceding embodiments, wherein the amount ranges from about 0.1% to about 15% by weight of a pharmaceutical composition or in such other amounts as described herein, including from about 1% to about 5%. BET inhibitors for use in the methods disclosed herein include at least one entity chosen from

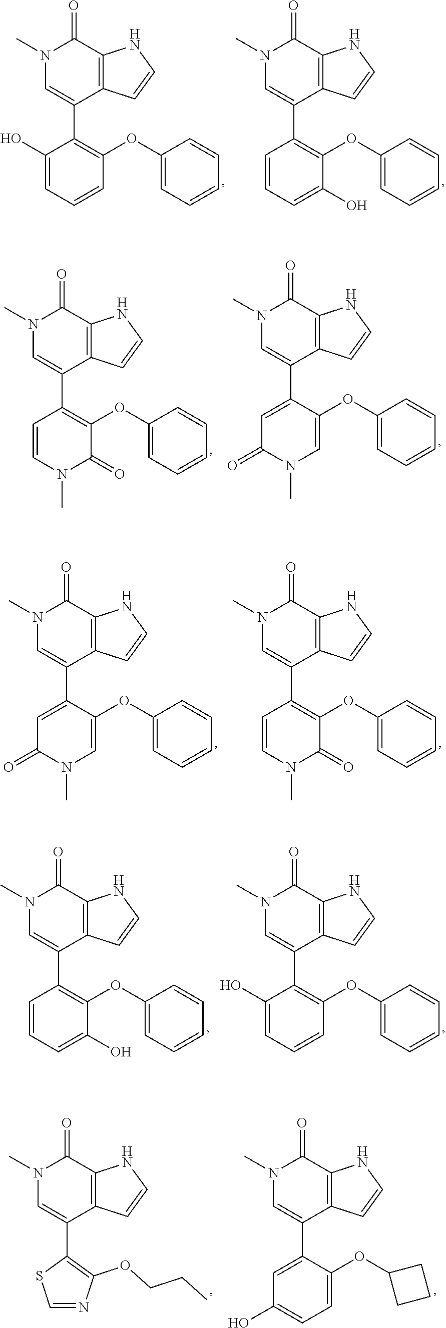

tautomers, stereoisomers, pharmaceutically acceptable salts, hydrates, and deuterated derivatives of any of the foregoing.

Disclosed herein are the use of any one of the preceding embodiments, wherein the composition comprises poly(lactic-co-glycolic acid) (PLGA) microspheres.

Disclosed herein are the use of any one of the preceding embodiments, wherein the PLGA microspheres degrade after administration to the subject.

Disclosed herein are the use of any one of the preceding embodiments, wherein the at least one entity is about 10% to about 80% w/w of the PLGA microspheres.

Disclosed herein are the use of any one of the preceding embodiments, wherein the at least one entity is about 25% w/w of the PLGA microspheres.

Disclosed herein are the use of any one of the preceding embodiments, wherein the composition further comprises at least one acid.

Disclosed herein are the use of any one of the preceding embodiments, wherein the at least one acid is hyaluronic acid.

Disclosed herein are the use of any one of the preceding embodiments, wherein the hyaluronic acid is cross-linked to at least one polymer.

Disclosed herein are the use of any one of the preceding embodiments, wherein the composition further comprises sodium carboxymethyl cellulose (NaCMC), NaCl, and water.

Disclosed herein are BET inhibitors for use in the treatment or prophylaxis of rheumatoid arthritis in a subject in need thereof comprising administering to a joint of a subject a composition comprising a therapeutically effective amount of a compound according to any one of the preceding embodiments.

Disclosed herein are the use of any one of the preceding embodiments, wherein the composition provides an initial release and/or a sustained release of the BET inhibitor at the joint.

Disclosed herein are the use of any one of the preceding embodiments, wherein the initial release of the BET inhibitor subsides, the composition continues to provide the sustained release of the BET inhibitor at the joint.

Disclosed herein are the use of any one of the preceding embodiments, wherein the administration of the composition to the joint is via an intra-articular or intra-venous injection.

Disclosed herein are the use of any one of the preceding embodiments, wherein after administration into the joint the initial and/or the sustained release of the BET inhibitor slows, arrests, reverses or otherwise inhibits structural damage to the joint tissue.

In one or more embodiments, a BETi or a pharmaceutically acceptable salt thereof is micronized. In one or more embodiments, it is encapsulated. In one or more embodiments, a BETi is encapsulated in particles, microparticles, nanoparticles, microcapsules, microspheres, nanocapsules, nanospheres, liposomes, niosomes, polymer matrices, silica-gels, graphite, nanocrystals, or microsponges.

In some embodiments, the total dose of BETi contained in the microparticles/vial is in a range selected from about 0.1-750 mg, 1-700 mg, 5-650 mg, 10-600 mg, 10-500 mg, 10-450 mg, 10-400 mg, 10-350 mg, 10-300 mg, 10-250 mg, 15-550 mg, 20-500 mg, 30-450 mg, 40-400 mg, 45-350 mg, 50-300 mg, 20-250 mg, 30-250 mg, 40-250 mg, 50-250 mg/vial, or from about 0.1-10 mg, 10-95 mg, 10-85 mg, 10-75 mg, 10-65 mg, 10-55 mg, 10-45 mg, 10-35 mg, 10-25 mg, 20-90 mg, 20-80 mg, 20-70 mg, 20-60 mg, 20-50 mg, 20-40 mg, 20-30 mg, 30-90 mg, 30-80 mg, 30-70 mg, 30-60 mg, 30-50 mg, 30-40 mg, 40-90 mg, 40-80 mg, 40-70 mg, 40-60 mg, 40-50 mg, 50-90 mg, 50-80 mg, 50-70 mg, 50-60 mg, 60-90 mg, 60-80 mg, 60-70 mg, 70-90 mg, 70-80 mg, and 80-90 mg, 90-100 mg, 100-120 mg, 120-140 mg, 140-160 mg, 160-180 mg, 180-200 mg, 200-220 mg, 220-240 mg, 240-260 mg, 260-280 mg, 280-300 mg, 300-320 mg, 320-340 mg, 340-360 mg, 360-380 mg, 380-400 mg, 400-420 mg, 420-440 mg, 440-460 mg, 460-480 mg, 480-500 mg, 500-520 mg, 520-540 mg, 540-560 mg, 560-580 mg, 580-600 mg, 600-620 mg, 620-640 mg, 640-660 mg, 660-680 mg, 680-700 mg, 700-720 mg, 720-740 mg, 740-760 mg, 760-780 mg, 780-800 mg/vial.

In some embodiments, the controlled or sustained release preparation or formulation is administered as one or more intra-articular injections. In some embodiments the injection can be intrasynovial, intrabursal or intraspinal. In some embodiments, the subject has arthritis, bursitis, Ehlers-Danlos syndrome, epicondylitis, Felty Syndrome, gouty arthritis, psoriatic arthritis, osteoarthritis, rheumatoid arthritis, Still's disease, tenosynovitis, synovitis, Sjögren's Syndrome, lyme disease, Whipple disease, bone cancer, lupus or other autoimmune joint disorders.

In one or more embodiments, the micronized particle size for a BETi is expressed as D50. By D50 is meant that the portions of particles with a size smaller than the D50 value are 50%. In some embodiments, the D50 for BETi is about 1-50 micrometers, e.g., about 5-25 microns.

In one or more other embodiments, the particle size for BETi is expressed as D90. By D90 is meant that the portion of particles with a size below the D90 value is 90%. In some embodiments, the D90 for micronized BETi is about 1-80 micrometers e.g., about 15-50 micrometers.

In some embodiments, the D90 particle size of micronized BETi is below about 80 microns, about 70 microns, about 60 microns, about 50 microns, about 40 microns, about 30 microns, about 20 microns, about 10 microns. In some embodiments about 50 microns, about 48 microns, about 46 microns, about 44 microns, about 42 microns, about 40 microns, about 38 microns, about 36 microns, about 34 microns, about 32 microns, about 30 microns, about 28 microns, about 30 microns, about 28 microns, about 26 microns, about 24 microns, about 22 microns, about 20 microns, about 18 microns, about 16 microns, about 14, microns, about 12 microns, about 10 microns, about 8 microns, about 7 microns, or about 6 microns. In some embodiments about 50 microns, about 45 microns, about 40 microns, about 35 microns, about 30 microns, about 25 microns, about 20 microns, about 15 microns, about 10 microns, or about 5 microns. In some embodiments, the D90 particle size of BETi is about 10 microns, about 9 microns, about 8 microns, about 7 microns, about 6, microns, about 5 microns, about 4 microns, about 3 microns, or about 2 microns. In some embodiments, the D90 particle size of BETi is about 1.5 microns, about 1 micron, about 0.9 microns, about 0.8 microns, about 0.7 microns, about 0.6, microns, about 0.5 microns, about 0.4 microns, about 0.3 microns, about 0.2 microns about, 0.1 micron. In some embodiments, the D90 particle size of BETi is in the nano particle size range of about less than 1 micron to about 0.03 microns or about 0.9 micron to about 0.05 microns.

In some embodiments, the D50 particle size of micronized BETi is below about 40 microns, about 30 microns, about 20 microns, about 10 microns, about 5 microns or about 1 micron. In some embodiments about 50 microns, about 48 microns, about 46 microns, about 44 microns, about 42 microns, about 40 microns, about 38 microns, about 36 microns, about 34 microns, or about 32 microns, about 30 microns, about 28 microns, about 30 microns, about 28 microns, about 26 microns, about 24 microns, about 22 microns, about 20 microns, about 18 microns, about 16 microns, about 14, microns, about 12 microns, about 10 microns, about 9 microns, about 8 microns, about 7 microns, about 6 microns, about 5 microns, about 4 microns, about 3 microns, about 2 or about 1 microns. In some embodiments about 50 microns, about 45 microns, about 40 microns, about 35 microns, about 30 microns, about 25 microns, about 20 microns, about 15 microns, about 10 microns, or about 5 microns. In some embodiments, the D50 particle size of BETi is about 10 microns, about 9 microns, about 8 microns, about 7 microns, about 6, microns, about 5 microns, about 4 microns, about 3 microns, about 2 microns, about 1 micron, about 0.5 microns, about 0.25 microns, or about 0.1 micron. In some embodiments, the D50 particle size of BETi is in the nano particle size range of about less than 1 micron to about 0.03 microns or about 0.9 micron to about 0.05 microns.

In one or more embodiments, the BETi is suspended in the carrier. In one or more embodiments, BETi or a pharmaceutically acceptable salt thereof is not micronized. In some embodiments, the D90 for the BETi or BETi salt is between about 100 and about 25 microns, e.g., about 100-80 microns, about 80-60 microns, about 60-40 microns, or about 35-25 microns, such as about 100, or about 90, or about 80, or about 70, or about 60, about 50, or about 40, about 30 microns or about 25 microns.

In one or more embodiments, BETi or a pharmaceutically acceptable salt thereof is not micronized. In some embodiments, the D50 for non-micronized BETi or BETi salt is about 8-60 micrometers e.g., about 30-60 micrometers. In some embodiments, the D50 particle size of the BETi or BETi salt is below about 60 microns, e.g., about 60 microns, about 58 microns, about 56 microns, about 54, about 52 microns, about 50 microns, about 48 microns, about 46 microns, about 44, microns, about 42 microns or about 40 microns. In some embodiments, it is below about 30 microns, e.g., about 29 microns, about 28 microns, about 27 microns, about 26 microns, about 25 microns, about 24 microns, about 23 microns, or about 22 microns. In some embodiments, it is below about 20 microns, e.g., about 19 microns, about 18 microns, about 17 microns, about 16 microns, about 15 microns, about 14 microns, about 13 microns, or about 12 microns. In some embodiments, it is below about 10 microns, e.g., about 9 microns, about 8 microns, about 7 microns, about 6 microns, about 5 microns, about 4 microns, about 3 microns, or about 2 microns.

In some embodiments, there is provided a composition comprising at least one compound disclosed herein and a carrier in which the at least one compound is suspended or substantially suspended. In one or more embodiments, the at least one compound is BETi. In some embodiments, there is provided a composition comprising a BETi and a carrier in which the BETi is dissolved or substantially dissolved.

In some embodiments, the at least one compound e.g., BETi, is suspended as nanoparticles. In some embodiments, the carrier comprises nanoparticles of at least one compound e.g., a BETi.

In some embodiments, at least about 95% of the at least one compound, e.g., BETi, is not present as agglomerates. In some embodiments, less than about 5%, or 4%, or 3%, or 2%, or 1% of the composition comprises agglomerates with at least one compound, e.g., a BETi. In some embodiments, the carrier composition is free of, essentially free of, or substantially free of at least one compound, e.g., BETi, agglomerates.

Pharmaceutical compositions comprising suspended BETi1 were found to be physically stable—with no degradation of BETi1 being observed for 3 m at 25° C.—and the compositions exhibited homogeneous distribution of the crystals and no aggregates were observed.

In some embodiments, the at least one compound e.g., BETi, is chemically stable e.g., for at least one month, or at least 2 months, or at least 3 months, or at least 6 months, or at least 9 months, or at least 12 months, or at least 15 months, or at least 18 months, or at least 21 months or at least 24 months. For example, in some embodiments, the at least one compound e.g., BETi, is chemically stable for at least 3 months at 25° C. For example, in some embodiments, the at least one compound e.g., BETi is chemically stable for at least 3 months at 40° C.

In some embodiments, at least 90% by mass of the at least one compound, e.g., BETi or salt thereof, is present in the composition when stored for 3 months at 25° C. In some embodiments, at least about 95% by mass of the at least one compound, e.g., BETi or salt thereof, is present in the composition when stored for 3 months at 25° C. In some embodiments, at least about 98% by mass of the at least one compound e.g., BETi or salt thereof, is present in the composition when stored for 3 months at 25° C. In some embodiments, at least 90% by mass of the at least one compound e.g., BETi or salt thereof, is present in the composition when stored for 3 months at 40° C. In some embodiments, at least about 95% by mass of the at least one compound e.g., BETi or salt thereof, is present in the composition when stored for 3 months at 40° C. In some embodiments, at least about 98% by mass of the at least one compound e.g., BETi or salt thereof, is present in the composition when stored for 3 months at 40° C.

In some embodiments, systemic exposure to at least one compound e.g., a BETi, applied topically or locally is much less than when the same amount is applied orally. In some embodiments, the systemic exposure is at least about 20-fold less. In some embodiments. In some embodiments, the systemic exposure is at least about 70-fold less, at least about 100-fold less, at least about 200-fold less, at least about 400-fold less or is at least about 500-fold less.

In one or more embodiments, the composition is a gel, paste, semi-solid, lotion, cream, soap, spray, mask, patch, liquid, powder, pomade, ointment, oil, foam, aerosol, liquid bolus, or mousse. In some embodiments, the composition is hydrophilic. In one or more embodiments, the composition is hydrophobic. In some embodiments, the composition is an emulsion. In one or more embodiments, the composition comprises hydrophobic oils and waxes. In one or more embodiments, the composition comprises fatty alcohols. In one or more embodiments, the composition comprises hydrophobic oils and waxes. In one or more embodiments, the composition comprises fatty acids. In one or more embodiments, the composition is surfactant-free.

In one or more embodiments, the composition comprising BETi is given prophylactically before the onset of symptoms associated with a BET-related condition or disorder (hereinafter “a BET related condition”). In one or more embodiments, the BETi composition is administered at the beginning of symptoms related to a BET-related condition. In one or more embodiments, the composition is administered during the first week, first two weeks, first three weeks, first month, first five weeks, first six weeks, first seven weeks, first eight weeks, first nine weeks, first ten weeks, first eleven weeks or first twelve weeks of symptoms related to a BET-related condition or some similar period, which could include parts of a week, such as one day, two days, three days, four days, five days, or six days. In one or more embodiments, the composition is administered one, two, three, four, five, six, seven, or eight weeks prior to the beginning of symptoms related to a BET-related condition. In some embodiments, the BETi composition is applied once daily. In some embodiments, a composition disclosed herein is administered once every two days or three days or four days or five days or six days or seven days for an initial period and thereafter at longer intervals say once every two weeks or monthly or quarterly or six monthly. In some embodiments the composition is applied twice daily. In some embodiments, the composition is applied at least once per day for at least 7 days. In some embodiments, a composition disclosed herein is administered at least once, twice or three times in a period of 7 days. In some embodiments, the composition is applied at least once per day for at least 14 days. In some embodiments, the composition is applied at least once per day for at least 21 days. In some embodiments, a composition disclosed herein is administered at least once, twice or three times in a period of 14 days. In some embodiments the composition is applied at least once per day for at least 4 weeks. In some embodiments, a composition disclosed herein is administered at least once, twice or three times in a period of at least 4 weeks. In some embodiments, the composition is applied at least once per day for at least 8 weeks. In some embodiments, a composition disclosed herein is administered at least once, twice or three times in a period of 8 weeks. In some embodiments, the composition is applied at least once per day for at least 12 weeks, at least 16 weeks, at least 20 weeks, at least 6 months, at least 12 months. In some embodiments, a composition disclosed herein is administered at least once, twice, three or four times in a period of 12 weeks, at least 16 weeks, at least 20 weeks, at least 6 months, at least 12 months.

In some embodiments, a composition disclosed herein is administered once a week for a month, or once a fortnight for one to two months, or once a month for a quarter, or once every two months for sixth months, or some other similar interval as is appropriate for the subject. In some embodiments, a composition disclosed herein is administered once daily on non-consecutive days. For example, in some embodiments, a composition disclosed herein is administered once daily on alternating days. In some embodiments, a composition disclosed herein is administered once on day 1, day 3, day 6, and day 9. In some embodiments, a composition disclosed herein is administered once on day 1, day 4, day 7, and day 10. In some embodiments, a composition disclosed herein is administered once daily on day 1, day 5, day 10, and day 15. In some embodiments, a composition disclosed herein is administered once on day 1, day 6, and day 12. In some embodiments the intervals between administration may be increasing sequentially. Examples include in some embodiments, a composition disclosed herein is administered once on day 1, day 3, day 6, day 10 and day 15. In some embodiments, a composition disclosed herein is administered once on day 1, day 4, day 8, day 13 and day 19. In some embodiments, a composition disclosed herein is administered once on day 1, day 8, day 22, day 50 and day 110. In some embodiments, a composition disclosed herein is administered on day 1, day 8, day 28, and day 56.

In some embodiments, the composition is applied as a maintenance dose following an initial treatment period. In some embodiments, for longer term, use a lower dose may be given. In some embodiments, the maintenance dose is applied on non-consecutive days or months. In some embodiments the maintenance dose is applied on alternative days or months. In some embodiments the maintenance dose is applied twice weekly or quarterly.

In one or more embodiments, BET-related conditions may include: an autoimmune disease, an immune system dysfunction, a viral disease, an allergic disease, a skin disease, an inflammatory pathway-related disease, an immune response, a hyperproliferative disorder, or a cancer.

In one or more embodiments, non-limiting examples of BET related conditions are PG, GPP, wounds, chronic wounds, hidradenitis suppurativa, lichen planus, diabetic foot ulcers, and pustulosis palmoplantaris.

In some embodiments, there is provided a method for preventing, treating or ameliorating symptoms related to a BET-related condition in a subject, comprising topically or locally administering, prior to symptoms, a composition disclosed herein e.g., a composition comprising BETi. In some embodiments, the administration of the composition disclosed herein is during the symptoms. In some embodiments, administration is continued for a period after alleviation of the symptoms, such as, one, two, three or four weeks afterwards. In some embodiments, the composition comprises at least one carrier and at least one BET inhibitor e.g., a BETi (free base, pharmaceutically acceptable salt, hydrate, deuterated derivative, etc.).

In some embodiments, there is provided a carrier composition suitable for providing delivery of at least one compound disclosed herein, e.g., BETi, locally or topically to the skin or to a mucosal membrane or to a body cavity surface or to the airways and lungs or to the lung parenchyma or to the alveoli. In some embodiments, the at least one compound disclosed herein is suspended or substantially suspended. In some embodiments, the at least one compound disclosed herein is partly suspended and partly dissolved. In some embodiments, the at least one compound disclosed herein is provided in a pharmaceutically effective amount (“PEA”). A PEA will depend on multiple factors, including the disorder to be treated or prevented, the active agent, the carrier, the delivery device or method and the subject. In some embodiments, a PEA could range from as little as about 0.0001% or about 0.001% to as high as about 5%. In some embodiments, a PEA could range from about 0.01% or about 0.1% to as high as about 15%. In some embodiments, a PEA is from about 0.001% to about 0.1% or from about 0.002% to about 0.08% by weight of the composition.

In some embodiments, there is provided a composition comprising at least one compound, tautomer, stereoisomer, pharmaceutically acceptable salt, hydrate, and/or deuterated derivative disclosed herein, e.g., BETi, and a carrier in which the at least one compound, tautomer, stereoisomer, pharmaceutically acceptable salt, hydrate, and/or deuterated derivative disclosed herein is suspended or substantially suspended. In some embodiments, at least about 99.9% of at least one compound, tautomer, stereoisomer, pharmaceutically acceptable salt, hydrate, and/or deuterated derivative disclosed herein is suspended in the composition. In some embodiments, there is provided a composition comprising a BETi and a carrier in which the BETi is dissolved or substantially dissolved. In some embodiments, at least about 99.9% of BETi is dissolved in the composition. In some embodiments, the BETi is provided as a powder. In some embodiments, the at least one compound, tautomer, stereoisomer, hydrate, and/or deuterated derivative is a pharmaceutically acceptable salt. In some embodiments, the at least one compound, tautomer, stereoisomer, pharmaceutically acceptable salt, hydrate, and/or deuterated derivative is chosen from pharmaceutically acceptable salts of Formula (I), tautomers, stereoisomers, hydrates, and deuterated derivatives thereof. In some embodiments, the pharmaceutically acceptable salt is a citrate salt, hydrochloride salt, hydrobromide salt, oxalate salt, nitrate salt, sulfate salt, phosphate salt, fumarate salt, succinate salt, maleate salt, besylate salt, tosylate salt, palmitate salt, tartrate salt, adipate salt, laurate salt, or myristate salt. In some embodiments, the at least one compound, tautomer, stereoisomer, pharmaceutically acceptable salt, hydrate, and/or deuterated derivative is homogeneously suspended and no agglomeration is detected. In some embodiments, the BETi is at least about 0.006 mg/mL. In some embodiments, the BETi is at least about 0.06 mg/mL. In some embodiments, the BETi is at least about 0.6 mg/mL. In some embodiments, the BETi is at least about 3 mg/mL. In some embodiments, the BETi is at least about 0.001 mg/mL, about 0.002 mg/mL, about 0.003 mg/mL, about 0.004 mg/mL, about 0.005 mg/mL, about 0.006 mg/mL, about 0.007 mg/mL, about 0.008 mg/mL or about 0.009 mg/mL. In some embodiments, the BETi is at least about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL or about 0.09 mg/mL. In some embodiments, the BETi is at least about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL or about 0.9 mg/mL. In some embodiments, the BETi is at least about 1 mg/mL, about 2 mg/mL, about 3 mg/mL, about 4 mg/mL, about 5 mg/mL, about 6 mg/mL, about 7 mg/mL, about 8 mg/mL, about 9 mg/mL or 10 mg/mL or between the range of about 0.06 mg/mL to about 6 mg/mL, or about 0.06 mg/mL to about 3 mg/mL, or about 0.6 mg/mL to about 6 mg/mL, or about 0.6 mg/mL to about 3 mg/mL.

In some embodiments, the at least one compound, tautomer, stereoisomer, pharmaceutically acceptable salt, hydrate, and/or deuterated derivative is present in an amount of at least about 0.001% by weight of the composition. In some embodiments, it is at least about 0.01%. In some embodiments, it is at least about 0.1%. In some embodiments, it is about 0.001% to about 1%. In some embodiments, it is at least about 1%. In some embodiments, it is 0.001% to about 0.9%, it is 0.001% to about 0.8%, it is 0.001% to about 0.7%, it is 0.001% to about 0.6%, it is 0.001% to about 0.5%, it is 0.001% to about 0.4%, it is 0.001% to about 0.3%. it is 0.001% to about 0.2%, it is 0.001% to about 0.1%. In some embodiments, it is about 0.01% to about 0.1%. In some embodiments, it is 0.01% to about 0.09%, it is 0.01% to about 0.08%, it is 0.01% to about 0.07%, it is 0.01% to about 0.06%, it is 0.01% to about 0.05%, it is 0.01% to about 0.04%, it is 0.01% to about 0.03%, it is 0.01% to about 0.02%, or it is 0.001% to about 0.01%. In some embodiments, it is about 0.1% to about 1%. In some embodiments, it is 0.1% to about 0.9%, it is 0.1% to about 0.8%, it is 0.1% to about 0.7%, it is 0.1% to about 0.6%, it is 0.1% to about 0.5%, it is 0.1% to about 0.4%, it is 0.1% to about 0.3%, it is 0.1% to about 0.2%, or it is 0.1% to about 0.015%. In some embodiments, it is about 0.002% to about 0.6%. In some embodiments, it is 0.002% to about 0.3%, or is about 0.002% to about 0.2%, or is about 0.002% to about 1.2%, or is about 0.04% to about 1.0%, or is about 0.45% to about 0.8% or is about 0.5% to about 0.75% by weight of the composition. In some embodiments it is about 0.1%, or about 0.15%, or about 0.2%, or about 0.25%, or about 0.3%, or about 0.35%, or about 0.4%, or about 0.45%, or about 0.5%, or about 0.55%, or about 0.6%, or about 0.65%, or about 0.7%, or about 0.75% or about 0.8%, or about 0.9% or about 1.0%, or about 1.1%, or about 1.2% by weight of the composition. In some embodiments, it is about 1.3%, or about 1.4%, or about 1.5%, or about 1.6%, or about 1.7%, or about 1.8%, or about 1.9%, or about 2.0%, or about 2.25%, or about 2.5%, or about 2.75%, or about 3.0%, or about 3.5%, or about 4.0% or about 4.5%, or about 5.0%, or about 6.0% by weight of the composition. In some embodiments, it is about 0.4% to about 1.8% by weight of the composition. In some embodiments, it is about 0.5% to about 1.75%, or about 0.6% to about 1.7%, or about 0.7% to about 1.7%, or about 0.5% to about 1.6%, or about 0.5% to about 1.5%, or about 0.5% to about 1.4%, or about 0.5% to about 1.3%, about 0.5% to about 1.2%, by weight of the composition. In some embodiments, it is about 0.5% to about 0.7% by weight of the composition. In some embodiments, it is about 0.5%, or about 0.6% or about 0.7% by weight of the composition. In some embodiments, the at least one compound is about 0.1% by weight of the composition. For example, when the at least one compound is a BETi salt, then about 0.1% by weight of the salt would provide a lower dose of BETi. In one or more embodiments, the active agent is present in an amount of any figure within the ranges provided herein. As would be understood by one of ordinary skill in the art in view of the disclosure herein, the amounts of “at least one compound, tautomer, stereoisomer, pharmaceutically acceptable salt, hydrate, and/or deuterated derivative” disclosed as % by weight of the composition are based on the weight of the free base of the compound, respectively. For example, when the at least one compound, tautomer, stereoisomer, pharmaceutically acceptable salt, hydrate, and/or deuterated derivative is a salt then about “0.1% by weight” would be the weight of the salt equivalent to 0.1% by weight of the free base.

In one or more embodiments, the BETi compound is formulated in a carrier suitable for topical or for local application. In one or more embodiments, the carrier is suitable for topical use, such as a gel, or a semi-solid, or a flowable semi-solid, or an ointment, or a liquid, or a foam, or a mousse, or a cream, or a lotion, or a solution. In some embodiments, the carrier and BETi is a provided as a powder. In some embodiments, it is lyophilized. In some embodiments, it is a provided as a suspension. In some embodiments, it is a provided as a solution. In some embodiments, it is a provided as micelles. In some embodiments, it is a provided as an emulsion. In some embodiments, it is delivered as a puff or aerosol. In some embodiments, it is delivered as a mist or a spray. In one or more embodiments, the carrier may be anhydrous. In one or more embodiments, the carrier may comprise water. In one or more embodiments, the carrier may be an emulsion. In some embodiments, the emulsion is with water and in some without. In other embodiments, the carrier is not an emulsion. In one or more embodiments, the carrier is a gel. In some embodiments, the gel comprises a silicone thickening agent. In some embodiments, the silicone thickening agent comprises a cross polymer and a silicone. In some embodiments, a gel comprises an elastomer-based formulation. In some embodiments, the gel comprises at least one polymeric agent, such as at least one gelling agent. In some embodiments, the gel comprises an oil or solvent and a polymeric agent, such as a gelling agent. In some embodiments, the gel is an oleogel formulation without elastomer.

In some embodiments, the gel comprises at least one polymeric thickening agent. In some embodiments, the at least one polymeric thickening agent comprises or is formed from at least one cross polymer. In some embodiments, the gel comprises a cellulose-based polymeric agent. In some embodiments, the gel comprises a PEG-based formulation. In some embodiments, the gel further comprises at least one emollient and/or at least one solvent.

In some embodiments, the BETi compound or salt thereof may be formulated in a carrier or vehicles suitable for application locally, e.g., to a joint, or, alternatively, to a body cavity, e.g., the lung. In some embodiments, the carrier is water based. In some embodiments, the carrier is anhydrous. In some embodiments, the composition is hydrophilic. In some embodiments, the composition may include an emollient. In some embodiments, the composition may include a humectant. In some embodiments, the emollient is hydrophilic. In some embodiments, the humectant is hydrophilic. In some embodiments, the hydrophilic composition comprises a hydrophilic solvent. In some embodiments, the hydrophilic solvent comprises a polar solvent or a polyol. In some embodiments, the hydrophilic composition comprises a diol, or a triol, or a polyol. In some embodiments, the hydrophilic composition comprises a saccharide (e.g., monosaccharide or disaccharide). In some embodiments, the hydrophilic composition comprises a sugar alcohol. In some embodiments, the hydrophilic composition comprises a polymeric agent. In some embodiments, the hydrophilic composition comprises a gelling agent. In some embodiments, the hydrophilic composition comprises a poloxamer. In some embodiments, the hydrophilic composition comprises a surfactant. In some embodiments, the surfactant is a non-ionic surfactant. In some embodiments, the hydrophilic composition comprises a protic solvent. In some embodiments, the hydrophilic composition comprises an aprotic solvent. In some embodiments, the hydrophilic composition comprises a modulating agent. In some embodiments, the hydrophilic composition comprises one or more of a preservative, or an antioxidant, or an ionization agent, or a chelating agent. In some embodiments, the hydrophilic composition comprises a buffering or pH agent. In some embodiments, the hydrophilic composition comprises a hydroxy acid.

In some embodiments, the compositions disclosed herein comprise at least one BETi. In some embodiments, BETi is micronized. In some embodiments, BETi is suspended as nanoparticles. In some embodiments, the carrier comprises nanoparticles of BETi. In some embodiments, the size range is expressed as D90 between about 2 m to about 50 μm. In some embodiments, the D90 is between about 5 m to about 50 μm. In some embodiments, the D90 is less than about 25 m, or is about 24 m, or about 22 m, or about 20 m, or about 18 m, or about 16 m, or about 14 m, or about 12 m or about 11 μm. In some embodiments, the D90 is less than about 10 m, or is about 9 m, or about 8 m, or about 7.5 m, or about 7 m, or about 6 m, or about 5 m or about 4 m, or about 3 μm. In one or more embodiments, the average uniform size range expressed as D90 is less than about 1 m, or less than about 0.75 m, or less than about 0.5 m, or less than about 0.25 m, or less than about 0.2 m, or is about 0.9 m, or about 0.8 m, or about 0.7 m, or about 0.6 m, or about 0.5 m, or about 0.4 m, or about 0.3 m, or about 0.25 m, or about 0.2 m, or about 0.15 m or about 0.1 μm.

In some embodiments, the BETi has some solubility in water. In some embodiments, the solubility of the BETi (e.g., BETi1) is about 0.005 mg/g in water. In other words, solubility is about 0.5 mg/100 ml water (ml=1 g). In some embodiments, the presence of low saline and CMC may not significantly change the solubility. Based on this and applying it to formulations #1-4 in Experimental Method N Section C for formulation #4 about 0.0004 g/100 g formulation (=0.4 mg/100 ml) will be dissolved, for formulation #3 about 12.5% of the 0.004 g/100 g (=4 mg/100 ml) formulation will be dissolved, for formulation #2 about 1% of the 0.04 g/100 g (=40 mg/100 ml) formulation will be dissolved and for formulation #1 about 0.1% of the 0.4 g/100 g (=400 mg/100 ml) formulation will be dissolved. Suspended formulations with a part dissolved may provide a reservoir effect as the solid dissolves over time.

In one or more embodiments, BETi is homogeneously dispersed at 0.001% to 4% strengths. In some embodiments, BETi is homogeneously dissolved at 0.001% to 0.1% strengths. In some embodiments, BETi is homogeneously dispersed at about 0.04%, about 10%, or to about 15% by weight. In one or more embodiments, the carrier or carrier components can reduce the potential for agglomeration of suspended BETi salt or base. In some embodiments, there is a reduction in the number of agglomerates. In some embodiments, there is a reduction in the size of the agglomerates. In some embodiments, there is a reduction in the frequency of agglomerates. In one or more embodiments, there is provided a carrier composition in which the number and size of any agglomerates is considered not significant. For example, in some embodiments, the average number of BETi particles in the size range between about 40 μm to about 100 m is less than about 50 per mg. In some embodiments, the average number of particles in the size range between about 100 μm and 200 m is less than about 10 per mg. In some embodiments, no or almost no particles larger than 200 μm are detected. In some embodiments, the average size of agglomerates is less than about 175 μm, or is less than about 150 μm, or is less than about 125 μm, or is less than about 100 μm, or is less than about 75 μm, or is less than about 50 μm. In some embodiments, at least about 95% of the BETi is not present as agglomerates. In some embodiments, less than about 5% of the composition comprises agglomerates. In some embodiments, less than about 4% of the composition comprises agglomerates. In some embodiments, less than about 3% of the composition comprises agglomerates. In some embodiments, less than about 2% of the composition comprises agglomerates. In some embodiments, less than about 1% of the composition comprises agglomerates. In one or more embodiments, the carrier composition is free of or essentially free of, or substantially free of agglomerates.

In one or more embodiments the carrier is water based. In one or more embodiments the carrier is anhydrous (or substantially so). In one or more embodiments the carrier comprises an emollient. In one or more embodiments the carrier comprises an elastomer. In one or more embodiments the carrier comprises a hydrophobic agent e.g., an oil. In one or more embodiments the carrier comprises one or more other ingredients suitable for topical or local delivery.

### Suitable Ingredients Assuming the Formulation is Water Based and May Comprise a Hydrophilic Solvent

In some embodiments, the water-based formulation comprises a hydrophilic solvent. In some embodiments, the hydrophilic solvent comprises a polar solvent or a polyol. A “polar solvent” is an organic solvent, typically soluble in both water and oil. Polar solvents, such as detailed below, possess high solubilizing capacity. Some polar solvents (e.g., DMSO) can solubilize oil. Examples of polar solvents include dimethyl isosorbide, polyols, such as glycerol (glycerin), propylene glycol, hexylene glycol, diethylene glycol, propylene glycol n-alkanols, terpenes, di-terpenes, tri-terpenes, limonene, terpene-ol, 1-menthol, dioxolane, ethylene glycol, other glycols, oleyl alcohol, alpha-hydroxy acids, such as lactic acid and glycolic acid, sulfoxides, such as dimethylsulfoxide (DMSO), dimethylformanide, methyl dodecyl sulfoxide, dimethylacetamide, azone (1-dodecylazacycloheptan-2-one), 2-(n-nonyl)-1,3-dioxolane, alkanols, such as dialkylamino acetates, and admixtures thereof. In some embodiments, the polar solvent is chosen from or is selected from the group consisting of dimethyl isosorbide, glycerol (glycerin), propylene glycol, hexylene glycol, terpene-ol, oleyl alcohol, lactic acid and glycolic acid.

In some embodiments, the polar solvent is a polyethylene glycol (PEG) or PEG derivative that is liquid at ambient temperature, including PEG 200 (MW (molecular weight) about 190-210 kD), PEG 300 (MW about 285-315 kD), PEG 400 (MW about 380-420 kD), PEG 600 (MW about 570-630 kD) and higher MW PEGs such as PEG 4000, PEG 6000 and PEG 10000 and mixtures thereof. Polyols are examples of polar solvents that can be contained in the carriers provided herein. A polyol is an organic substance that contains at least two hydroxy groups in its molecular structure.

In one or more embodiments, the carrier comprises at least one diol (i.e., a compound that contains two hydroxy groups in its molecular structure). In some embodiments, the carrier comprises at least one monohydric alcohol (i.e., a compound that contains one hydroxy group in its molecular structure). In some embodiments, the diol is a glycol. In some embodiments, the glycol is propylene glycol and/or hexylene glycol. Examples of diols include propylene glycol (e.g., 1,2-propylene glycol and 1,3-propylene glycol), butanediol (e.g., 1,2-butanediol, 1,3-butanediol, 2,3-butanediol and 1,4-butanediol), butanediol (e.g., 1,3-butanediol and 1,4-butenediol), butynediol, pentanediol (e.g., pentane-1,2-diol, pentane-1,3-diol, pentane-1,4-diol, pentane-1,5-diol, pentane-2,3-diol and pentane-2,4-diol), hexanediol (e.g., hexane-1,6-diol hexane-2,3-diol and hexane-2,56-diol), hexylene glycol, octanediol (e.g., 1,8-octanediol), neopentyl glycol, 2-methyl-1,3-propanediol, diethylene glycol, triethylene glycol, tetraethylene glycol, dipropylene glycol, and dibutylene glycol.

In some embodiments, the carrier contains at least one triol (a compound that contains three hydroxy groups in its molecular structure), such as glycerin, butane-1,2,3-triol, butane-1,2,4-triol, and hexane-1,2,6-triol.

In some embodiments, a polyol refers to a mixture of polyols. In some embodiments, the mixture of polyols may contain at least one diol and at least one triol. In some embodiments, the mixture of polyols may contain at least two diols or at least triols. In some embodiments, the polyol is selected from glycerin, hexylene glycol, and propylene glycol. In one or more embodiments, the polyol is glycerin. In some embodiments, the polyol is hexylene glycol. In some embodiments, the polyol is propylene glycol. In some embodiments, the polyol is propylene glycol and glycerin. In some embodiments, the polyol is hexylene glycol and glycerin. In some embodiments, the polyol is a combination of propylene glycol and glycerin.

In some embodiments, the formulation comprises one or more saccharides or sugar alcohols. In some embodiments, part of a mixture of polyols is a saccharide. Exemplary saccharides include, but are not limited to monosaccharide, disaccharides, oligosaccharides and sugar alcohols.

A monosaccharide is a simple sugar that cannot be hydrolyzed to smaller units. Empirical formula is (CH2O)n and range in size from trioses (n=3) to heptoses (n=7). Exemplary monosaccharide compounds are ribose, glucose, fructose and galactose.

Disaccharides are made up of two monosaccharides joined together, such as sucrose, maltose and lactose.

A sugar alcohol (also known as a polyol, polyhydric alcohol, or polyalcohol) is a hydrogenated form of saccharide, whose carbonyl group (aldehyde or ketone, reducing sugar) has been reduced to a primary or secondary hydroxyl group. They are commonly used for replacing sucrose in foodstuffs, often in combination with high intensity artificial sweeteners to counter the low sweetness. Some exemplary sugar alcohols, which are suitable for use according to the present disclosure, are mannitol, sorbitol, xylitol, maltitol, lactitol. (Maltitol and lactitol are not completely hydrogenated compounds—they are a monosaccharide combined with a polyhydric alcohol.) Mixtures of polyols, including (1) at least one polyol selected from a diol and a triol; and (2) a saccharide are contemplated within the scope.

In some embodiments, the composition includes a hydrophilic solvent, and water. In some embodiments, the solvent includes water and a water miscible organic solvent, which. by way of non-limiting examples, is a polyhydroxy compound and/or a poly-ethoxylated compound.

In some embodiments suitable polyhydroxy solvents (polyols) are chosen from small organic molecules having two or more hydroxy groups on their carbon skeleton, such as ethylene glycol, propylene glycol, glycerol, butanediols and isomers thereof, pentaerythritol, sorbitol, mannitol, dimethyl isosorbide, polyethylene glycol, polypropylene glycol, diethylene glycol monoethyl ether (Transcutol®) and mixtures thereof.

In some embodiments poly-ethoxylated compounds may enhance the effectiveness of BETi by dissolving BETi. Examples of suitable poly-ethoxylated compounds include polyethylene glycol (e.g., PEG 400), tetrahydrofurfuryl alcohol polyethyleneglycol ether (glycofurol, tetraglycol (TG)). Among the above-mentioned water miscible solvents, suitable compounds include transcutol, polyethylene glycol and TG and mixtures thereof.

In some embodiments, the solvent is water or a water miscible organic solvent, such as a polyhydroxy compounds and poly-ethoxylated compounds. In one embodiment, the composition has a water-to-water miscible organic solvent ratio of about 1:10 to about 10:1. Due to toxicity of lower alkyl alcohols, the water miscible compound is not a lower alkyl, e.g., C₁-C₅, alcohol. In some embodiments, the composition, e.g., the water miscible composition, may comprise a short chain alcohol. In some embodiments, the short chain alcohol e.g., ethanol is less than about 40% by weight of the composition. In some other embodiments, e.g., due to the potential toxicity or side effects of lower alkyl alcohols when administered to the lungs, the composition e.g., water miscible composition, does not include or is substantially free of a lower alkyl, e.g., C₁-C₅, alcohol.

In some embodiments, the composition is an aqueous hydrophilic composition. In some embodiments, the aqueous hydrophilic composition includes a polymeric agent e.g., a gelling agent. In some embodiments, suitable gelling agents may include, in a non-limiting manner, naturally-occurring polymeric materials such as locust bean gum, guar gum, sodium alginate, sodium caseinate, egg albumin, gelatin agar, carrageenin gum, sodium alginate, xanthan gum, quince seed extract, tragacanth gum, starch, chemically modified starches and the like, semi-synthetic polymeric materials such as cellulose ethers (e.g. hydroxyethyl cellulose, methyl cellulose, carboxymethyl cellulose, hydroxy propylmethyl cellulose), hydroxypropyl guar gum, soluble starch, cationic celluloses, cationic guars and the like and synthetic polymeric materials such as carboxyvinyl polymers, polyvinylpyrrolidone, polyvinyl alcohol, polyacrylic acid polymers, polymethacrylic acid polymers, polyvinyl acetate polymers, polyvinyl chloride polymers, polyvinylidene chloride polymers, and the like.

Also, useful herein are gelling agents such as the acrylic acid/ethyl acrylate copolymers and the carboxyvinyl polymers sold, for example, by the B.F. Goodrich Company under the trademark of Carbopol® resins. These resins include a colloidal water-soluble polyalkenyl polyether cross linked polymer of acrylic acid cross linked with from 0.75% to 2% of a cross linking agent, such as polyallyl sucrose or polyallyl pentaerythritol. Examples include Carbopol 934, Carbopol 940, Carbopol 950, Carbopol 980, Carbopol 951 and Carbopol 981. Carbopol 934 is a water-soluble polymer of acrylic acid cross linked with about 1% of polyallyl ether of sucrose having an average of about 5.8 allyl groups for each sucrose molecule.

In one or more embodiments, the polymeric e.g., gelling agents are present in concentrations that are suitable for application through inhalation. For example, where the composition is an aqueous composition, the amounts will, in one or more embodiments, be below the levels to form a gel or semi solid composition, e.g., so that the composition can be delivered, e.g., as a mist.

In some embodiments, the carrier comprises a Poloxamer. Poloxamer is a synthetic block copolymer of ethylene oxide and propylene, having the general formula of:

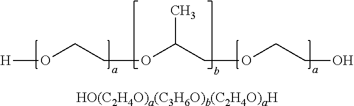

For the generic term “Poloxamer”, these copolymers are commonly named with the letter “P” (for Poloxamer) followed by three digits, the first two digits×100 give the approximate molecular mass of the polyoxypropylene core, and the last digit×10 gives the percentage polyoxyethylene content. For example, P407 is a Poloxamer with a polyoxypropylene molecular mass of 4,000 g/mol and a 70% polyoxyethylene content.

Poloxamers have several useful characteristics depending on the poloxamer(s) selected. They are polymeric. They can be thickeners and, at higher concentrations, gel forming in aqueous solution. They also have surfactant properties and can act as emulsifiers and wetting agents. Some are able to create thermo-sensitive solutions, such that their properties, such as viscosity, change with temperature. They are strongly hydrophilic and can help to improve solubility. Poloxamers are soluble in water and in alcohol. Poloxamers are used as emulsifying agents for intravenous fat emulsions, as solubilizing agents to maintain clarity in elixirs and syrups and as wetting agents for anti bacterials. They have been used in a variety of oral parenteral, and topical pharmaceutical formulations, and are generally regarded as nontoxic and non-irritant. Poloxamers are stable materials and aqueous solutions of Poloxamers are stable in the presence of acids, bases and metal ions.

In some embodiments the aqueous vehicles or carriers comprise a BETi encapsulated, for example, in microspheres, comprising a poloxamer. In some embodiments, there are provided formulations in which the poloxamer improves the solubility of or acts to dissolve an active agent in an aqueous phase, wherein such an active agent is not fully soluble in said aqueous phase.

As used herein, the term “fixing” means a viscosity change of a formulation containing poloxamer upon a temperature change such that the viscosity of a preparation comprising poloxamer can, in some embodiments, substantially increase when the temperature changes from room temperature of about 20° C. to a temperature of about 30° C. or more. In some embodiments, “fixing” may also be induced by a change in the pH or in the ionic strength of the said solution. One consequence of fixing is that a liquid or semi liquid formulation turns viscous enough to remain substantially in the same place when applied to a body surface. The term “fixing poloxamer” relates to a poloxamer, which is capable, subject to exposure of elevated temperature, change in pH or ionic strength, of affording a “fixing” effect.

Poloxamers are available in different grades which vary from liquids to solids. Examples Poloxamers are: 124, 181, 182, 183, 184, 185, 188, 212, 215, 217, 231, 234, 235, 237, 238, 331, 333, 334, 335, 338, 401, 402, 403 and 407.

In some embodiments, no surfactant other than poloxamer is required to make a gel. The gel may be fixing such that it is liquid at room temperature and gel or semi gel like at or approaching body temperature, even at very low poloxamer concentrations. In some embodiments other surfactants may be present. In further embodiments a combination of poloxamer and polymer can be used. In some embodiments one or more surfactants other than poloxamers are used. In some embodiments a combination of two or more surfactants are used, wherein at least one has a HLB above 9 and one has a HLB below 9.

In some embodiments, the composition comprises a poloxamer in combination with a polysaccharide to produce a synergistic gelling effect. This is a unique advantage, which enables much lower amounts of poloxamer and polysaccharide to be used to achieve a gelling effect. Thus, whilst poloxamer alone (e.g., poloxamer 407) or polysaccharide alone (e.g., xanthan gum) can produce aqueous gels, higher amounts are required than the total amount when they are in combination. In some embodiments, in order to attain the fixing property, the Poloxamer is selected such that the formulation may be liquid or semi liquid at room temperature, but, upon warming to body temperature, the viscosity increases. In one or more embodiments, the poloxamer and amounts are selected such that there is no fixing effect at body temperature. In some embodiments, the increase in viscosity is sufficient to have a gelling effect or to increase the retention time at the site of application.

In some embodiments, in addition to poloxamer or as an alternative the composition may contain a single surface active agent having an HLB value between about 9 and about 19, or more than one surface active agent and where the weighted average of their HLB values is between about 9 and about 19.

In some embodiments, the surface-active agent (other than a poloxamer) has a hydrophilic lipophilic balance (HLB) between about 9 and about 14, or the composition contains more than one surface active agent and the weighted average of their HLB values is between about 9 and about 14.

In some embodiments, the surface-active agent e.g., in addition to poloxamer, includes at least one non-ionic surfactant.

Non limiting examples of possible non-ionic surfactants include polysorbates, such as polyoxyethylene (20) sorbitan monostearate (Tween 60) and poly(oxyethylene) (20) sorbitan monooleate (Tween 80); poly(oxyethylene) (POE) fatty acid esters, such as Myrj 45, Myrj 49, Myrj 52 and Myrj 59; poly(oxyethylene)alkyl ethers, such as poly(oxyethylene) cetyl ether, poly(oxyethylene) palmityl ether, polyethylene oxide hexadecyl ether, polyethylene glycol ether, brij 38, brij 52, brij 56, brij W1, ceteareth 20; sucrose esters, partial esters of sorbitol and its anhydrides, such as sorbitan monolaurate and sorbitan monolaurate; mono or diglycerides, isoceteth-20, and mono-, di- and tri-esters of sucrose with fatty acids (sucrose esters including those having high monoester content, which have higher HLB values). Mixtures of non-ionic surfactants are contemplated.

Pulmonary surfactants are essential for life as they line the alveoli to lower surface tension in the liquid/air interface, which prevents the alveoli from filling with fluid or collapsing with the expulsion of air during exhalation. They are also responsible for killing inhaled germs and prevented them from spreading and modulating the immune response.

In some embodiments, the aqueous formulations comprise one or more lung surfactants chosen from one or more of phospholipids such as dipalmitoylphosphatidylcholine, surfactant-associated proteins such as SP-A, SP-B, SP-C, and SP-D. In some embodiments, the aqueous formulations comprise surfactant chosen from one or more of beractant, calfactant and poractant alpha.

In some embodiments, the BETi compounds lack solubility in a range of solvents, carriers, and formulations and can be suspended and dispersed homogeneously therein. Homogenous suspensions thereof are exemplified herein. In some embodiments, the BETi compounds are also soluble in a range of other solvents carriers and formulations.

In some embodiments, a protic solvent acts to improve solubility of a BETi compound. In some embodiments, the delivery of a BETi compound is improved by the aprotic solvent and/or protic solvent. In some embodiments, there is provided a composition comprising an aprotic polar solvent and/or a protic polar solvent, in which the protic polar solvent can be a short chain alcohol.

In some embodiments, the composition comprises a solvent which can help dissolve a BETi compound, wherein said solvent includes one or more of or is selected from the group consisting of water, ethyl acetate, acetonitrile, IPAc, t-Butyl methyl ether, heptane, dichloromethane or anisole.

In some embodiments, the composition is free, essentially free, or substantially free of a solvent which can dissolve BETi, wherein said solvent includes one or more of or is selected from the group consisting of water, ethyl acetate, acetonitrile, IPAc, t-Butyl methyl ether, heptane, dichloromethane or anisole.

### Suitable Ingredients Assuming the Formulation is Anhydrous (or Substantially so) and Hydrophilic.

In some embodiments, the carrier, includes a waterless solvent, and one or more of a stabilizing surfactant, a polymeric agent and a modulating agent. In some embodiments, hydrophilic solvents, such as polyethylene glycol or propylene glycol, are used as waterless solvents in a waterless system. These hydrophilic solvents, while not as polar a water, nonetheless provide a degree of polarity that is useful in solubilizing polar or hydrophilic active agents. In one or more embodiments, the solvent or secondary solvent is a polyol.

In general, for liquid PEG based compositions, in one or more embodiments the composition comprises a liquid PEG e.g., PEG 200, PEG 300, PEG 400, and PEG 600. In some embodiments, the hydrophilic solvent can comprise a higher molecular weight PEG. In some embodiments, the hydrophilic solvent is PEG comprising a high molecular weight PEG chosen from one or more of PEG 1000, PEG 1500, PEG 4000, PEG 6000 and PEG 8000, and the balance of the PEG is chosen from or is selected from the group consisting of PEG 200, PEG 300, PEG 400, and PEG 600. In some embodiments, the PEG is a combination of a high and low molecular weight PEG chosen from or is selected from the group consisting of PEG6000/PEG200; PEG400/PEG1500, PEG4000/PEG200; PEG4000/PEG400, PEG 4000/PEG 600, PEG4000/PEG400/PEG200; and the like. In one or more embodiments, where PEGs are used in liquid formulations, the types and amounts of the PEGs will, in one or more embodiments, be below the levels to form a gel or semi solid composition, e.g., so that the composition can be delivered, e.g., as a mist.

In some embodiments, a secondary solvent may be optionally added to the composition. The secondary solvent is selected from a variety of organic solvents that are typically miscible on both water and oil. Examples of solvent that can be used in the carrier include dimethyl isosorbide, tetrahydrofurfuryl alcohol polyethyleneglycol ether (glycofurol), DMSO, pyrrolidones, (such as N-Methyl-2-pyrrolidone and 1-Methyl-2-pyrrolidinone), ethyl proxitol, dimethylacetamide (DMAc), PEG-type surfactants and alpha hydroxy acids, such as lactic acid and glycolic acid.

In some embodiments, the polyol comprises or is selected from the group consisting of propylene glycol, butylene glycol. hexylene glycol and glycerin (and mixtures thereof); and the secondary polar solvent comprises or is selected from the group consisting of dimethyl isosorbide, diethylene glycol monoethyl ether, a liquid polyethylene glycol, and glycofurol.

In some embodiments, the carrier contains (1) at least one polyol; and (2) dimethyl isosorbide. In some embodiments, when hydrophilic solvent is propylene glycol (PG), the composition includes a surface active agent, e.g. a steareth surface active agent and a polymeric agent or gelling agent, e.g., hydroxypropylcellulose, and is free or substantially free of oil, e.g., silicone oil.

In some embodiments, the second hydrophilic solvent is present in the composition and is a polyol chosen from or is selected from the group consisting of a diol, a triol, and a saccharide, wherein the triol is chosen from or is selected from the group consisting of glycerin, butane-1,2,3-triol, butane-1,2,4-triol, and hexane-1,2,6-triol, and wherein the diol is chosen from or is selected from the group consisting of propylene glycol, butanediol, butenediol, butynediol, pentanediol, hexanediol, octanediol, neopentyl glycol, 2-methyl-1,3-propanediol, diethylene glycol, triethylene glycol, tetraethylene glycol, dipropylene glycol, dibutylene glycol, and mixtures of any two or more thereof.

In some embodiments, the additional component is a humectant. Suitable humectants include, but are not limited to, guanidine, urea, glycolic acid, glycolate salts, ammonium glycolate, quaternary alkyl ammonium glycolate, lactic acid, lactate salts, ammonium lactate, quaternary alkyl ammonium lactate, aloe vera, aloe vera gel, allantoin, urazole, alkoxylated glucose, hyaluronic acid, lactamide monoethanolamine, acetamide monoethanolamine and derivatives, esters, salts, and mixtures of any two or more thereof.

In some embodiments, the composition comprises an aprotic solvent, wherein the aprotic solvent is an organic solvent that does not contain an O—H or N—H bond; or does not exchange protons with a substance dissolved in it. In the context herein, the aprotic polar solvent is a solvent with a comparatively high relative permittivity (or dielectric constant), greater than about 15, and a sizable permanent dipole moment, that cannot donate suitably labile hydrogen atoms to form strong hydrogen bonds; and it is miscible in water. Examples of aprotic polar solvents, suitable according to the present disclosure include, but are not limited to dimethyl sulfoxide (DMSO), dimethylformamide (DMF), acetonitrile, acetone, methyl ethyl ketone, 1,4-Dioxane and tetrahydrofuran (THF). Additional non-limiting examples include N-methylpyrrolidone, pyridine, piperidine, dimethyl ether, hexamethylphosphorotriamide, dimethylformanide, methyl dodecyl sulfoxide, N-methyl-2-pyrrolidone and 1-methyl-2-pyrrolidinone) and azone (1-dodecylazacycloheptan-2-one), and mixtures of any two or more thereof.

The provision and selection of polymeric agent is however not straightforward. In one or more embodiments, the polymers are miscible or swell in the waterless solvent. In some embodiments, the polymeric agent is a modified cellulose. In some embodiments, the modified cellulose is a lower molecular weight cellulose polymer derivative. In some embodiments, the polymeric agent is hydroxypropyl cellulose. In another embodiment, the polymeric agent is a carbomer such as Carbopol 934®.

The polymeric agent may serve to control drug residence in the target organ or tissue (joint or lung). Preferably, the polymeric agent is soluble or readily dispersible in the polyol; or in the mixture of a polyol and an additional polar solvent.

Non-limiting examples of polymeric agents that are soluble or readily dispersible in propylene glycol are hydroxypropylcellulose and carbomer (homopolymer of acrylic acid is crosslinked with an allyl ether pentaerythritol, an allyl ether of sucrose, or an allyl ether of propylene, such as Carbopol® 934, Carbopol® 940, Carbopol® 941, Carbopol® 980 and Carbopol® 981).

Other polymeric agents are suitable for use according to the present disclosure provided that they are soluble or readily dispersible in the polyol; or, in the mixture of a polyol and an additional polar solvent, on a case by case basis.

Exemplary polymeric agents include, in a non-limiting manner, naturally-occurring polymeric materials, such as locust bean gum, sodium alginate, sodium caseinate, egg albumin, gelatin agar, carrageenin gum, sodium alginate, xanthan gum, quince seed extract, tragacanth gum, guar gum, cationic guars, hydroxypropyl guar gum, starch, amine-bearing polymers such as chitosan; acidic polymers obtainable from natural sources, such as alginic acid and hyaluronic acid; chemically modified starches and the like, carboxyvinyl polymers, polyvinylpyrrolidone, polyvinyl alcohol, polyacrylic acid polymers, polymethacrylic acid polymers, polyvinyl acetate polymers, polyvinyl chloride polymers, polyvinylidene chloride polymers and the like.

Additional exemplary polymeric agents include semi-synthetic polymeric materials, such as cellulose ethers, such as methylcellulose, hydroxypropyl cellulose, hydroxypropyl methylcellulose, hydroxyethyl cellulose, hydroxy propylmethyl cellulose, methylhydroxyethylcellulose, methylhydroxypropylcellulose, hydroxyethylcarboxymethyl-cellulose, carboxymethyl cellulose, carboxymethylcellulose carboxymethylhydroxyethyl-cellulose, and cationic celluloses. Polyethylene glycol, having molecular weight of 1000 or more (e.g., PEG 1,000, PEG1500 PEG 4,000, PEG 6,000 and PEG 10,000), also have gelling capacity and, while they are considered herein as “secondary polar solvents”, as detailed herein, they are also considered polymeric agents.

In some embodiments, a surface active agent is present in the composition. In one or more embodiment, the surface active agent is a polysorbate, including polysorbate 80 and Tween 80. In one or more embodiments, a combination of surfactants is present in the composition. Specific non-limiting examples of surfactant systems are combinations of liquid polysorbate surfactants and PEG compounds, such as Tween 80/PEG-40 stearate; methyl glucaso sequistearate; polymeric emulsifiers, such as Pemulen (TRI or TR2); liquid crystal systems, such as Arlatone (2121), Stepan (Mild RM1), Nikomulese (41), Montanov (68), and the like.

The term modulating agent is used to describe an agent which can improve the stability of or stabilize a carrier or composition and/or an active agent by modulating the effect of a substance or residue present in the carrier or composition. The substance or residue may, for example, be acidic or basic and potentially alter an artificial pH in a waterless, or substantially non-aqueous or environment or it may be one or more metal ions which may act as a potential catalyst in a waterless, or substantially non-aqueous environment.

In some embodiments, the modulating agent is used to describe an agent which can affect pH in an aqueous solution. The agent can be any of the known buffering systems used in pharmaceutical or cosmetic formulations as would be appreciated by a man of the art. It can also be an organic acid, a carboxylic acid, a fatty acid an amino acid, an aromatic acid, an alpha or beta hydroxyl acid an organic base or a nitrogen-containing compound.

In some embodiments, the modulating agent is used to describe an agent, which is a chelating or sequestering or complexing agent that is sufficiently soluble or functional in the waterless solvent to enable it to “mop up” or “lock” metal ions.

In some embodiments, the modulating agent is used to describe an agent which can affect pH in an aqueous solution. The term modulating agent more particularly means an acid or base or buffer system or combinations thereof, which is introduced into or is present in and acts to modulate the ionic or polar characteristics and any acidity or basicity balance of a waterless or substantially non-aqueous carrier, or composition.

In some embodiments, the modulating agent may also be a preservative or an antioxidant or an ionization agent. Any preservative, antioxidant, or ionization agent suitable for pharmaceutical or cosmetic application may be used. Non-limiting examples of antioxidants are tocopherol succinate, propyl galate, butylated hydroxy toluene and butyl hydroxy anisol. In one or more embodiments, the modulating agent is a flavonoid. Ionization agents may be positive or may be negative depending on the environment and the active agent or composition that is to be protected. Ionization agents may, for example, act to protect or reduce sensitivity of active agents. Non-limiting examples of positive ionization agents are benzyl conium chloride and cetyl pyridium chloride. Non-limiting examples of negative ionization agents are sodium lauryl sulphate, sodium lauryl lactylate and phospholipids.

In some embodiments, the composition further comprises one or more modulating agents, and are, for example, selected from the group of triethanol amine, sodium citrate, and citric acid.

In some other embodiments the composition may be free, essentially free or substantially free of modulating agents.

### Hydroxy Acids

In one or more embodiments, the composition may comprise a hydroxy acid. Exemplary hydroxy acids include alpha- or beta-hydroxy acids, poly-hydroxy acids, or any combinations of any of the foregoing. The hydroxy acid can be an alpha-hydroxy acid. Non-limiting examples of alpha hydroxy acids include, but are not limited to, glycolic acid, lactic acid, malic acid, citric acid, alpha-hydroxyethanoic acid, alpha-hydroxyoctanoic acid, alpha-hydroxycaprylic acid, hydroxycaprylic acidglycolic acid, tartaric acid, pyuric acid, citric acid, as well as their corresponding salts and pharmaceutically-acceptable derivatives; or any combination of any of the foregoing. The hydroxy acid can be a beta-hydroxy acid. Non-limiting examples of beta-hydroxy acids include, but are not limited to, salicylic acid, beta hydroxybutanoic acid, tropic acid, and trethocanic acid.

### Miscellaneous Examples of Suitable Ingredients/Excipients in One or More Embodiments for the Disclosed Formulations/Compositions

In one or more embodiments, ingredients suitable for respiratory routes of administration are chosen from or are selected from the group comprising 1,2-distearoyl-sn-glycero-3-phosphocholine, alcohol, anhydrous citric acid, anhydrous trisodium citrate, apaflurane, ascorbic acid, benzalkonium chloride, black ink, calcium carbonate, calcium chloride, carrageenan, cetylpyridinium chloride, chlorobutanol, citric acid monohydrate, dichlorodifluoromethane, dichlorotetrafluoroethane, edetate disodium, ferric oxide yellow, fluorochlorohydrocarbons, gelatin, glycerin, glycine, hydrochloric acid, hypromellose 2906 (4 mpa·s), lactose, lactose monohydrate, lecithin (e.g., soybean), magnesium stearate, mannitol, menthol, methylparaben, nitric acid, norflurane, n-phenyl-1-napthylamine, nutmeg oil, oleic acid, petrolatum, phenylethyl alcohol, polysorbate 80, potassium chloride, propylene glycol, propylparaben, saccharin, saccharin sodium, silicon dioxide, sodium bicarbonate, sodium bisulfite, sodium chloride, sodium hydroxide, sodium lauryl sulfate, sodium metabisulfite, sodium sulfate anhydrous, sorbitan trioleate, sulfuric acid, thymol, titanium dioxide, trichloromonofluoromethane, trisodium citrate dihydrate, tromethamine, turpentine oil, zinc oxide and mixtures of any two or more thereof as would be appreciated by one skilled in the art. In addition to being suitable for respiratory routes the aforesaid ingredients may also be used for compositions for other topical applications e.g., to the skin and/or to lesions and/or for areas of fibrosis other than pulmonary and for local applications e.g., intraarticular application.

In some embodiments, ingredients/excipients suitable for use in the composition or carrier to treat or prevent a fibrosis or respiratory disorder or disease and are compatible for use, e.g., by inhalation into the lung, chosen from antioxidants/preservatives/chelating agents/salts comprising or selected from a group consisting of n-phenyl-1-napthylamine, ascorbic acid, benzalkonium chloride, calcium carbonate, calcium chloride, edetate disodium, methylparaben, phenylethyl alcohol, potassium chloride, propylparaben, sodium bisulfite, sodium chloride, sodium metabisulfite and thymol; buffers or pH agents comprising or selected from a group consisting of anhydrous citric acid, anhydrous trisodium citrate, citric acid monohydrate, hydrochloric acid, nitric acid, sodium hydroxide, sulfuric acid, trisodium citrate dihydrate and tromethamine; color agents/pigments comprising or selected from a group consisting of black ink, ferric oxide yellow, titanium dioxide and zinc oxide; dispersant such as silicon dioxide; drying agent such as sodium sulfate anhydrous; fatty acid such as oleic acid; hydrophobic solvent such as nutmeg oil or petrolatum; polar solvents sugar alcohol/polyol comprising or selected from a group consisting of alcohol, glycerin, menthol, propylene glycol, lactose, lactose monohydrate and mannitol; preservative such as chlorobutanol; propellant comprising or selected from a group consisting of a fluorochlorohydrocarbon propellant, apaflurane, dichlorodifluoromethane, dichlorotetrafluoroethane, norflurane and trichloromonofluoromethan; a salt such as sodium bicarbonate; solvent\solubilizing agent such as glycine and/or turpentine oil; surfactant phospholipid comprising or selected from a group consisting of 1,2-distearoyl-sn-glycero-3-phosphocholine, cetylpyridinium chloride, polysorbate 80, sodium lauryl sulfate, sorbitan trioleate, lecithin and soybean; sweetener such as saccharin and saccharin sodium; thickening agent comprising or selected from a group consisting of carrageenan, gelatin, a hypromellose e.g., 2906 (4 mpa·s) and magnesium stearate. In addition to being suitable for respiratory routes the aforesaid ingredients may also be used for compositions for other topical applications e.g., to the skin and/or to lesions and/or for areas of fibrosis other than pulmonary and for local applications e.g., intraarticular application.

Although these ingredients have been grouped into various general categories, some ingredients may also fit into other categories.

In one or more embodiments, ingredients/excipients suitable for use in the composition or carrier to treat or prevent a joint disorder or disease or to treat or prevent fibrosis or respiratory disease or disorder and are compatible for use e.g., by injection into the joint e.g., intra articular, intra synovial, intra spinal and/or intra bursal, or, alternatively, inhalation into the lung, are chosen from acetic acid, anhydrous trisodium citrate, anhydrous dextrose, benzalkonium chloride, benzyl alcohol, carboxymethylcellulose sodium, citric acid monohydrate, creatinine, dibasic potassium phosphate, edetate disodium, hyaluronate sodium, hydrochloric acid, methylcellulose, methylparaben, miripirium chloride, monobasic potassium phosphate, phenol, phenylethyl alcohol, phosphoric acid, polyethylene glycol 3350, polyethylene glycol 4000, polyglactin, polysorbate 80, propylparaben, sodium acetate, sodium bisulfite, sodium chloride, sodium hydroxide, sodium metabisulfite, sodium phosphate, sodium phosphate, dibasic, anhydrous, sodium phosphate, dibasic, heptahydrate, sodium phosphate, monobasic, sodium phosphate, monobasic, monohydrate, sodium sulfite, sorbitol, dextrose, and trisodium citrate dihydrate and mixtures of two or more thereof as would be appreciated by one skilled in the art. In some embodiments they may be chosen to be introduced into a formulation by way of illustration for one or more of the following objectives e.g., as “viscosupplemention agents”, such as hyaluronate sodium, as one or more of “antioxidants”/“preservatives”/“chelating agents”/“salts”, such as, sodium bisulfite, sodium metabisulfite, sodium sulfite, edetate disodium, benzyl alcohol, methylparaben, phenylethyl alcohol, propylparaben, benzalkonium chloride, phenol, and miripirium chloride, and sodium chloride, as “buffers” or “pH agents”, such as anhydrous trisodium citrate, citric acid monohydrate, dibasic potassium phosphate, hydrochloric acid, monobasic potassium phosphate, phosphoric acid, sodium acetate, sodium hydroxide, sodium phosphate monobasic monohydrate, trisodium citrate dihydrate, sodium phosphate dibasic anhydrous, acetic acid, sodium phosphate, sodium phosphate monobasic, and sodium phosphate dibasic heptahydrate, as “surfactants” such as polysorbate 80, as “polymeric agents” e.g., carboxymethylcellulose sodium, methylcellulose, polyglactin, e.g., “polar rehydration agents”, such as sorbitol, and anhydrous dextrose (which can be polar solvents in solution and for example may be used with saline), or as “thickening agents” such as polyethylene glycol 3350, and polyethylene glycol 4000. In some embodiments creatinine as an excipient may be useful for intra-articular compositions. In addition to being suitable for respiratory routes the aforesaid ingredients may also be used for compositions for other topical applications e.g., to the skin and/or to lesions and/or for areas of fibrosis other than pulmonary and for local applications e.g., intraarticular application.

In some embodiments, the composition comprises a carrier and one or more active pharmaceutical ingredients (active agents). In some embodiments the active agent comprises a BET inhibitor. In some embodiments the active agent comprises a pan BET inhibitor. In some embodiments the active agent comprises a selective BET inhibitor. In some embodiments the active agent is a combination of a pan BET inhibitor (e.g., a BETi) and a selective BET inhibitor. In one or more embodiments the pan BET inhibitor is a “soft” pan BET inhibitor.

In some embodiments, the carrier is oil based. In some embodiments, the carrier is petrolatum based. In some embodiments, the carrier is water based. In some embodiments, the carrier comprises an emulsion.

In some embodiments, the BETi compositions avoid one or more unwanted side effects and adverse effects that can occur with administration of steroids and other treatments. For example, adverse reactions of triamcinolone injections include, hypersensitivity reactions, such as anaphylaxis, joint infection and damage, increased risk of infections, alterations in endocrine function, cardiovascular and renal effects, increased intraocular pressure, gastrointestinal perforation, alternations in bone density and behavioral and mood disturbances. As yet another example, adverse reactions of dexamethasone include, fluid and electrolyte disturbances, musculoskeletal, gastrointestinal, neurologic, dermatologic, endocrine, ophthalmic, metabolic cardiovascular, anaphylactoid or hypersensitivity reactions, thromboembolism, weight gain, increased appetite, and nausea.

The BETi composition described herein can in some embodiments avoid undesirable side effects such as the ones described above. In some embodiments, the BETi compositions herein can provide an effective, safe, well tolerated long term solutions to treat joint and joint related diseases and disorders unlike steroids which are preferably limited for use for short periods and are not well tolerated and can have undesirable side effects as indicated herein.

In some embodiments, there is provided a composition comprising a BETi1 or a pharmaceutically acceptable salt thereof and a carrier in which BETi is suspended to treat or lessen the symptoms of a joint or joint related disorder or disease or of a secondary joint disorder or disease.

In some embodiments, the carrier or vehicle is a hydrophilic composition. In some embodiments, it is a water based hydrophilic composition. In some embodiments, the hydrophilic water based composition comprises more than about 50%, or >about 55%, or >about 60%, or >about 65%, or >about 70%, or >about 75%, or >about 80%, or >about 85%, or >about 90%, or >about 95% water by weight or volume of the composition. In some embodiments, it is waterless or anhydrous hydrophilic composition. In some embodiments, the hydrophilic composition is essentially waterless or anhydrous. In some embodiments, the hydrophilic composition is a substantially waterless or anhydrous composition. In some embodiments, the hydrophilic composition is a low composition. In some embodiments, the hydrophilic based composition comprises less than about 50%, or <about 45%, or <about 40%, or <about 35%, or <about 30%, or <about 25%, or <about 20%, or <about 15%, or <about 10%, or about >5%.

Silicone can, in some embodiments, be a suitable carrier for the BETi compounds herein. In some embodiments, the composition is free, essentially free or substantially free of a silicone.

In some embodiments, the introduction of at least one polymeric agent (e.g., gelling agent) in formulations comprising BETi may further help the formulation and/or the treatment. In some embodiments, the polymeric agent is biodegradable. In some embodiments, the polymeric agent comprising agent is formulated to provide a viscosity suitable for injection. In some embodiments, the viscosity is such as to allow the formulation to be flowable through the needle or injector. In some embodiments, the formulation is formulated to provide a viscosity compatible with the joint or joint fluid (e.g., synovial fluid) so as to allow for easy movement of the joint. In some embodiments, the formulation is formulated to provide a viscosity compatible with the joint or joint fluid (e.g., synovial fluid) so as to allow for support and/or lubrication of the joint. In some embodiments, upon application, the gel is to form a protective covering or in some embodiments an adhesive film over the area.

In some embodiments, the polymers may be cured ex-vivo forming a solid matrix that incorporates the drug for controlled release to an inflammatory region. Suitable biodegradable polymers may be natural or synthetic. Natural biocompatible biodegradable polymers include, but are not limited to, proteins, such as albumin, collagen, gelatin, synthetic poly(aminoacids), and prolamines; glycosaminoglycans, such as hyaluronic acid and heparin; and polysaccharides, such as alginates, chitosan, starch, and dextrans. Synthetic biocompatible biodegradable materials include, but are not limited to, poly(lactide-co-glycolide) (PLGA), polylactide (PLA), polyglycolide (PG), polyhydroxybutyric acid, poly(trimethylenecarbonate), polycaprolactone (PCL), polyvalerolactone, poly(alpha-hydroxy acids), poly(lactones), poly(amino-acids), poly(anhydrides), polyketals poly(arylates), poly(orthoesters), polyurethanes, polythioesters, poly(orthocarbonates), poly(phosphoesters), poly(ester-co-amide), poly(lactide-co-urethane, polyethylene glycol (PEG), polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA blends and copolymers thereof and any combinations thereof. The biocompatible biodegradable material can include a combination of biocompatible biodegradable materials. For example, the biocompatible biodegradable material can be a triblock, or other multi-block, formation where a combination of biocompatible biodegradable polymers is joined together.

In one or more embodiments, the carrier comprises at least one elastomer and at least one emollient. A detailed list of emollients is provided below. In some embodiments, emollient includes one or more of a mineral oil, glyceride oil, a branched-chain ester, and a branched hydrocarbon oil. In some embodiments, the emollient includes one or more of a triglyceride oil, an isopropyl ester, and a saturated and branched hydrocarbon oil. In some embodiments, the carrier is free, essentially free or substantially free of at least one elastomer. In some embodiments, the carrier is free, essentially free or substantially free of at least one emollient.

### Hydrophilic Emollients

Certain hydrophilic solvents listed herein, such as glycerin, various glycols (for example propylene glycol), hydroxy acids and PEGs (for example PEG, 200, 400, 600, 4000, 6000, 10,000) attract water and consequently possess emollient properties. In some embodiments, the hydrophilic emollients may comprise a polar humectant or a polar moisturizer such as a polyol, a polyethylene glycol (PEGs), a polypropylene glycol (PG), urea, sorbitol and/or hydroxy acids.

Polyethylene glycol of average molecular weight 1500 (PEG 1500) is a solid and melts at about 45 degrees C. In some embodiments the high molecular weight PEG is 1500. In some embodiments it can be used in combination with one or more of PEG 200, PEG 400 and PEG 600. The carrier according to the present disclosure can contain a single PEG or a mixture of two or more PEGs. PEGs having molecular weight of more than about 1000 possess gelling properties, i.e., they increase the viscosity of a composition. Therefore, by combining PEGs with different molecular weights/melting points, one can attain varying levels of flowability as desirable for the treatment of a given target site. Exemplary PEGs include, without limitation, PEG 200, PEG 300, PEG 400, PEG 600, PEG 1000, PEG 4000, PEG 6000, and PEG 8000. The carrier, according to the present disclosure, can contain a single PEG or a mixture of two or more PEGs. In some embodiments, the polyol comprises a liquid PEG having a molecular weight of about 600 or less. In some embodiments, the polyol comprises a solid PEG having a molecular weight of about 1000 or more. Solid PEGs can contribute to providing a composition with an increased viscosity. In some embodiments, the composition is substantially free, essentially free, or free of a PEG.

Exemplary PGs, without limitation, include 1,2-propylene glycol and 1,3-propylene glycol.

In some embodiments the composition comprises a humectant. In some embodiments, the humectant is hydrophilic. Exemplary humectants are propylene glycol, propylene glycol derivatives, and glycerin. Further humectants include but are not limited to guanidine, urea, glycolic acid, glycolate salts, ammonium glycolate, quaternary alkyl ammonium glycolate, lactic acid, lactate salts, ammonium lactate, quaternary alkyl ammonium lactate, aloe vera, aloe vera gel, allantoin, urazole, alkoxylated glucose, hyaluronic acid, lactamide monoethanolamine, acetamide monoethanolamine and derivatives, esters, salts and mixtures thereof.

In one or more embodiments, the carrier is not hydrophilic. In some embodiments, the carrier is not hydrophobic. In some embodiments, the carrier is free of or substantially free of hydrophilic compounds. In some embodiments, the carrier is free of or substantially free of hydrophobic compounds. In some embodiments, the carrier is free of or substantially free of volatile hydrophobic compounds. In some embodiments, the carrier is free of or substantially free of volatile hydrophilic compounds, which in some embodiments includes a volatile hydrophilic propellant. In some embodiments, the carrier is hydrophilic. In some embodiments, the carrier is free of or substantially free of non-hydrophilic compounds. In some embodiments, the carrier is free of or substantially free of volatile non-hydrophilic compounds. In some embodiments, the carrier comprises a surfactant e.g., a polysorbate, such as polysorbate 80. In some embodiments, the carrier is free of or, essentially free of, or is substantially free of a surfactant. In some embodiments, the carrier comprises water. In some embodiments, the carrier is free of essentially free of, or substantially free of water. In some embodiments, the carrier comprises one or more preservatives, such as benzyl alcohol or a paraben. In some embodiments, the carrier is free of, or essentially free of, or substantially free of preservatives. In some embodiments, the carrier comprises one or more anti-oxidants, such as sodium bisulfite, or sodium metabisulfite. In some embodiments, the carrier is free of, or essentially free of, or substantially free of anti-oxidants. In some embodiments, the carrier comprises one or more scavengers. In some embodiments, the carrier is free of, or essentially free of, or substantially free of scavengers. In some embodiments, the carrier comprises one or more additional stabilizers. In some embodiments, the carrier is free of, essentially free of, or substantially free of additional stabilizers. In some embodiments, the carrier comprises one or more chelating agents. In some embodiments, the carrier is free of, essentially free of, or substantially free of chelating agents. In some embodiments, the carrier comprises one or more biocompatible biodegradable antioxidants, preservatives, chelating agents, or salts such as benzyl alcohol, edetate disodium, methylparaben, phenylethyl alcohol, propylparaben, sodium bisulfite, sodium metabisulfite, sodium sulfite, benzalkonium chloride, sodium chloride, phenol or miripirium chloride. In some embodiments, the carrier comprises one or more biocompatible biodegradable buffers or pH agents such as anhydrous trisodium citrate, citric acid monohydrate, dibasic potassium phosphate, hydrochloric acid, monobasic potassium phosphate, phosphoric acid, sodium acetate, sodium hydroxide, sodium phosphate monobasic monohydrate, trisodium citrate dihydrate, sodium phosphate dibasic anhydrous, acetic acid, sodium phosphate, sodium phosphate monobasic, sodium phosphate dibasic heptahydrate. In some embodiments, the carrier comprises one or more biocompatible biodegradable surfactants such as polysorbate 80. In some embodiments, the carrier comprises one or more biocompatible biodegradable polymers such as carboxymethylcellulose sodium, methylcellulose or polyglactin. In some embodiments, the carrier comprises one or more biocompatible biodegradable polar rehydration agents such as sorbitol or dextrose. In some embodiments, the carrier comprises one or more biocompatible biodegradable thickening agents such as polyethylene glycol 3350, polyethylene glycol 4000. In some embodiments, the carrier comprises one or more biocompatible biodegradable viscosupplemention agents such as a hyaluronate or a hyaluronate derivative or fragment. In some embodiments, the carrier comprises one or more biocompatible biodegradable lubricant agents such as a lubricin or a lubricin derivative or fragment. Lubricin is chondrocyte-secreted glycoprotein that primarily conducts or facilitates boundary lubrication between joint surfaces. In some embodiments, the carrier comprises a lubricin or a lubricin derivative or fragment together with a hyaluronate or hyaluronate derivative or fragment.

In one or more embodiments, the carrier comprises a penetration enhancer that does not dissolve a BETi. In some embodiments, the carrier comprises a penetration enhancer that only essentially dissolves the BETi. In one or more embodiments, the carrier comprises a penetration enhancer that only substantially dissolves the BETi. In some other embodiments, the carrier comprises a penetration enhancer that dissolves part or proportion of the BETi such that part of the dose is suspended and part is in solution. In some embodiments, the carrier comprises a penetration enhancer that essentially dissolves BETi. In some embodiments, the carrier comprises a penetration enhancer that dissolves at least about 90% of the BETi. In some embodiments, the carrier comprises a penetration enhancer that dissolves at least about 95% of the BETi. In some embodiments, the carrier comprises a penetration enhancer that dissolves at least about 99% of the BETi. In some embodiments, the carrier comprises a penetration enhancer that dissolves at least about 100% of the BETi.

In one or more embodiments the carrier is free or substantially free of a penetration enhancer that dissolves a proportion of the BETi. In some embodiments, the proportion of the total BETi that the penetration enhancer or the compound may dissolve is at least about 15%. In some embodiments, it is at least about 10%, or at least about 7.5%, or at least about 5%, or at least about 2.5%, or at least about 1%, or at least about 0.7%, or at least about 0.6%, or at least about 0.5%, or at least about 0.4%, or at least about 0.3%, or at least about 0.2%, or at least about 0.1%, or at least about 0.05%, or at least about 0.01%, or at least about 0.005%, or at least about 0.001%. In some embodiments, it is about 0.1% or more. In some embodiments, it is about 0.01% or more. In some embodiments, it is about 0.001% or more. In some embodiments, the proportion of the total BETi that the penetration enhancer or the compound may dissolve is less than about 25% or, about <20%, or about <15%, or <about 10%, or, about <5%, or about <2%, or about <1%, or about <0.2%, or about <0.02%.

In one or more embodiments, the total amount of BETi that is dissolved in the carrier or composition is less than about 15% by weight of the total composition. In some embodiments, the total amount of BETi that is dissolved in the carrier or composition is less than about 10%, or less than about 7.5%, or less than about 5%, or less than about 2.5%, or less than about 1%, or less than about 0.7%, or less than about 0.6%, or less than about 0.5%, or less than about 0.4%, or less than about 0.3%, or less than about 0.2%, or less than about 0.1%, or less than about 0.05%, or less than about 0.01%, or less than about 0.005%, or less than about 0.001%, or less than about 0.0001%, or less than about 0.00015%, or less than about 0.0002%, or less than about 0.0003%. In some embodiments, it is between about 0.1% and about 0.01%. In some embodiments, it is between about 0.01% and about 0.001%. In some embodiments, it is between about 0.1% and about 0.001%. In some embodiments, it is between about 0.001% and about 0.0002%.

BETi1 was found to be freely soluble in tetrahydrofurane and 1,4-dioxane, sparingly soluble in polyethylene glycol 400, methanol, ethanol, acetone and methyl ethyl ketone; slightly soluble in isopropanol and insoluble in water, ethyl acetate, acetonitrile, IPAc, t-Butyl methyl ether, heptane, dichloromethane or anisole. In some embodiments, the composition includes a short chain alcohol. In some embodiments, the short chain alcohol comprises some of ethanol, methanol or isopropanol. In some embodiments, the composition includes an aprotic polar solvent. In some embodiments, the aprotic polar solvent comprises one or more of methyl ethyl ketone, acetone, tetrahydrofuran or 1,4-Dioxane. In some embodiments, the composition includes a hydrophilic solvent. In some embodiments the hydrophilic solvent includes one or more of dimethyl isosorbide, glycerin, ethanol, propylene glycol, butylene glycol, PEG 200, hexylene glycol, PEG 400, dimethyl sulfoxide or diethylene glycol monoethyl ether.

In one or more embodiments a compound that can dissolve or dissolves a portion of a BETi includes one or more of tetrahydrofuran, 1,4-Dioxane, methanol, polyethylene glycol 400, acetone, methyl ethyl ketone, ethanol or isopropanol. In some embodiments, the composition or carrier comprises a solvent chosen from one or more of tetrahydrofuran, 1,4-Dioxane, methanol, polyethylene glycol 400, acetone, methyl ethyl ketone, ethanol or isopropanol.

In one or more embodiments a compound that does not dissolve or poorly dissolves a BETi includes one or more of water, ethyl acetate, acetonitrile, IPAc, t-Butyl methyl ether, heptane, dichloromethane or anisole. In some embodiments, the composition or carrier is free from some of tetrahydrofuran, 1,4-Dioxane, methanol, polyethylene glycol 400, acetone, methyl ethyl ketone, ethanol or isopropanol.

In one or more embodiments, the composition is occlusive, e.g., is petrolatum based. In one or more embodiments, the composition is petrolatum based. Petrolatum was observed to have an impact on MMP-9 release, increasing it and although petrolatum did not have major effects on gene expression GADD45A was induced and WNT16 was down regulated in the RHE-model so petrolatum may be less beneficial in vitiligo on its own as a carrier. In one or more embodiments petrolatum may nevertheless be a suitable component of a carrier for the BETi compounds described herein, for example, in a carrier formulation comprising a silicone, an oil, or other emollients. In some embodiments the oil is a plant oil, in some embodiments an essential oil, in some embodiments a mineral oil, and in some embodiments an animal or fish oil. In some embodiments petrolatum is less than about 60%, or less than about 50%, or less than about 40%, or less than about 30%, or less than about 20%, or less than about 10% by weight of the composition. In some embodiments the composition is free or substantially free of petrolatum. In one or more embodiments, the composition is partially occlusive. In one or more embodiments, the carrier is free or substantially free of compounds to which BETi is not inert. In one or more embodiments, the carrier is lipophilic. In one or more embodiments, the lipophilic carrier comprises at least one oil that is liquid at room temperature. In one or more embodiments, the lipophilic carrier comprises at least one oil that is solid at room temperature. In one or more embodiments, the lipophilic carrier comprises at least one oil that is liquid at room temperature and at least one oil that is solid at room temperature. In one or more embodiments, the carrier comprises a polymeric agent. In one or more embodiments, the polymeric agent is a gelling agent. In one or more embodiments, the carrier comprises a gelling agent and a hydrophobic agent or oil. In one or more embodiments, the carrier comprises at least one elastomer. In some embodiments, the carrier is lipophobic. In some embodiments, the lipophobic carrier comprises at least one solvent that is liquid at room temperature. In some embodiments, the lipophobic carrier comprises at least one solvent that is solid at room temperature. In some embodiments, the lipophobic carrier comprises at least one solvent that is liquid at room temperature and at least one solvent that is solid at room temperature. In some embodiments, the carrier comprises at least one polymeric agent. In some embodiments, the at least one polymeric agent is chosen from gelling agents. In some embodiments, the carrier comprises at least one gelling agent and at least one hydrophilic agent or solvent. In some embodiments, the carrier comprises at least one PEG.

In one or more embodiments, there is provided a composition comprising a BETi as a salt, wherein the salt is more stable than the base.

In some embodiments, there is provided a composition wherein the composition is physically stable from about 8° C. to about 40° C., or from about 10° C. to about 35° C., or from about 15° C. to about 30° C., or from about 20° C. to about 25° C., e.g., the viscosity of the composition is stable or substantially stable from about 8° C. to about 40° C. In some embodiments, the viscosity of the composition is stable or substantially stable from about 10° C. to about 35° C. In some embodiments, the viscosity of the composition is stable or substantially stable from about 15° C. to about 30° C. In some embodiments, viscosity, of the composition is stable or substantially stable from about 20° C. to about 25° C.

In one or more embodiments, there is provided a composition wherein the viscosity of the composition is stable or substantially stable from about 8° C. to about 40° C. In some embodiments, the viscosity of the composition is stable or substantially stable from about 10° C. to about 35° C. In some embodiments, the viscosity of the composition is stable or substantially stable from about 15° C. to about 30° C. In some embodiments, viscosity, of the composition is stable or substantially stable from about 20° C. to about 25° C.

In some embodiments, the carrier comprises a gelled oil. In some embodiments, the carrier comprises a gelled mineral oil. In some embodiments, the carrier comprises a gelled mineral oil and an elastomer. In some embodiments, the carrier comprises an elastomer and an emollient. In some embodiments, the carrier comprises a gelled oil and an emollient. In some embodiments, the carrier comprises an elastomer, a gelled oil and an emollient. In some embodiments, the gelled oil comprises a mineral oil. In some embodiments, the emollient is one or more of a glyceride oil, a branched alkyl ester, and a branched hydrocarbon oil. In some embodiments, if present, the glyceride oil comprises a triglyceride oil, the branched alky ester comprises an isopropyl ester, and the branched hydrocarbon oil is saturated. In some embodiments, the triglyceride oil comprises an MCT oil.

In some embodiments, at least about 99.9% of BETi is suspended. In some embodiments, at least about 99%, about 98%, about 97%, about 96%, or about 95% of the active agent is suspended.

In some embodiments, the carrier or carrier base is a gel or comprises a gelled oil. In some embodiments, the oil is a silicone oil and the gelling agent is a cross polymer. In some embodiments, the oil is a mineral oil and the gelling agent is a polymer and/or a copolymer, such as ethylene/propylene/styrene copolymer or butylene/ethylene/styrene copolymer. In some embodiments, the gelled mineral oil comprises a Versagel®.

In some embodiments, the carrier comprises an emollient. In some embodiments, it comprises a combination of two or more emollients. In some embodiments, the emollient comprises one or more of a mineral oil, a glyceride, a triglyceride, a diglyceride, a monoglyceride, an MCT oil, a branched hydrocarbon oil, a saturated and branched hydrocarbon oil, squalene, squalane, a branched alkyl ester, isopropyl isostearate, isopropyl palmitate, isopropyl myristate, oleyl alcohol, a mineral oil, a vegetable oil, a liquid fatty acid, a liquid fatty alcohol, a branched liquid fatty acid, a branched liquid fatty alcohol, glyceryl monooleate, glyceryl isostearate, glyceryl dicaprate, a polypropylene glycerol alkyl ether, a polypropylene glycerol stearyl ether, polypropylene glycerol 15 stearyl ether, polypropylene glycerol 11 stearyl ether, glycerol behenate, diisopropyl adipate, cetearyl ethylhexanoate, and cetearyl isononanoate. In some embodiments, the emollient comprises one or more triglyceride oils. In some embodiments, the triglyceride oil comprises MCT oil. In some embodiments, the sole emollient is MCT oil. In other embodiments, it is combined with an alkyl ester. In some embodiments, the emollient comprises a branched alkyl ester. In some embodiments, the branched alkyl ester comprises an isopropyl ester or a glycerol iso-ester. In some embodiments, the isopropyl ester comprises isopropyl isostearate, isopropyl palmitate, isopropyl myristate or mixtures of two or more thereof. In some embodiments, the isopropyl ester comprises isopropyl isostearate. In some embodiments, the triglyceride oil is combined with a hydrocarbon oil. In some embodiments, the emollient comprises a branched hydrocarbon oil. In some embodiments, the branched hydrocarbon oil comprises squalene and/or squalane. In some embodiments, the emollient comprises a branched and saturated hydrocarbon oil, such as squalane. In some embodiments, the emollient comprises at least two of a triglyceride oil, an isopropyl ester and a saturated and branched hydrocarbon oil. In some embodiments, the emollient comprises at least two of isopropyl isostearate, squalane and an MCT oil. In some embodiments, the emollient comprises a triglyceride oil, an isopropyl ester and a saturated and branched hydrocarbon oil. In some embodiments, the emollients comprise MCT oil, an isopropyl ester and squalane. In some embodiments, the isopropyl ester comprises isopropyl isostearate.

In one or more embodiments, a branched alkyl ester such as isospropyl isostearate may be substituted by or complemented with by the addition of one or more of the following: isostearyl isostearate, oleyl oleate, isocetyl stearate, hexyl laurate, isostearyl neopentanoate, ethylhexyl stearate, octyldodecyl neopentanoate, cetearyl octanoate, isodecyl neopentanoate, decyl oleate, isononyl ethylhexanoate, isononyl isononanoate, hexyldecyl ethylhexanoate, isotridecyl isononanoate, cetyl ethylhexanoate, octyldodecyl neodecanoate, octyldodecyl myristate, hexyldecyl isostearate, ethylhexyl hydroxystearate, octyldodecyl stearoyloxystearate, diisopropyl dilinoleate, octyl isopalmitate, isodecyl oleate, and octyl palmitate.

In one or more embodiments a branched hydrocarbon oil, such as squalene, may be substituted by or alternatively complemented with by the addition of one or more of the following: squalene, pristane, a mineral oil, a hydrogenated polyisobutene, isohexadecane, isodecane, or isododecane, and branched alkanes.

In one or more embodiments, triolein or lorenzo's oil may also be used.

In one or more embodiments, the composition comprising an elastomer and at least one emollient can provide two, three, or four of the following characteristics: an improvement in the chemical stability of BETi, when applied topically to skin or mucosa, an increased delivery into the skin or mucosa; when applied topically to skin or mucosa, a reduced delivery through the skin or mucosa; and, when applied topically to skin, an increased delivery into the epidermis and reduced delivery through the skin.

In one or more embodiments, the BETi, e.g., BETi1 is in an effective concentration sufficient to bind to BDI and BDII domains of BET proteins in the dermis or epidermis in the applied area of skin or lesion of a mammal. In one or more embodiments, the BETi, e.g., BETi1 is in an effective concentration sufficient to bind to BDI and BDII domains of BET proteins within a joint or area of the joint of a mammal e.g., when injected into the joint. In one or more embodiments, the BETi, e.g., BETi1 is in an effective concentration sufficient to bind to BDI and BDII domains of BET proteins in the alveoli, lung parenchyma or lung tissue in the applied area of a mammal. In one or more embodiments, the BETi, e.g., BETi1 is in an effective concentration sufficient to bind to BDI and BDII domains of BET proteins in the mucosal tissue in the applied area of a mammal. In one or more embodiments, the mammal is a human subject. In some embodiments, the BETi is in an effective concentration sufficient to reach an apparent maximum inhibition of BDII in the administered area of a mammal, (e.g., lung parenchyma or lung tissue or a mucosa or the skin (dermis/epidermis/follicles), as indicated when a significant additional increase in the BETi concentration by weight % in the composition does not result in a significant increase in efficacy in treating a disorder. In some embodiments, the BETi is in an effective concentration sufficient to reach an apparent maximum inhibition of BDII in the dermis or epidermis in the applied area of a human subject, as indicated when a significant additional increase in BETi concentration by weight % in the composition does not result in a significant increase in efficacy in treating a disorder. In some embodiments, the BETi is in an effective concentration after reaching an apparent maximum binding of BDII, e.g., in the applied area of a human subject (e.g., lung parenchyma or lung tissue or a mucosa or the skin (dermis/epidermis/follicles), can result in a consequential increase in binding to BDI leading in turn to a significant decrease in efficacy in treating a disorder despite the higher dose. In some embodiments, BETi is in an effective concentration sufficient to reach a plateau effect in the applied area of a mammal, such as a human (e.g., lung parenchyma or lung tissue or a mucosa or the skin (dermis/epidermis/follicles). In some embodiments, an effective concentration is about 0.1% to about 15% by weight or in such other amounts as described herein, such as about 0.05% to about 15%, such as about 0.01% to about 10%, about 0.1% to about 10%, or about 1% to about 10%. In some embodiments, an effective concentration is about 0.1% by weight or less. In some embodiments, an effective concentration is about 0.06% by weight or less. In some embodiments, an effective concentration is about 0.006% by weight or less. In one or more embodiments, an effective concentration is about 0.1% by weight. In one or more embodiments, the BETi comprises or is BETi1.

In some embodiments, the carrier is free or substantially free of one, two, or more of water, surfactants, hydrophilic compounds, preservatives, anti-oxidants, scavengers, chelating agents and additional stabilizers. In some embodiments, the composition is anhydrous or substantially anhydrous. Alternatively, in some embodiments, the carrier comprises one or more component chosen from water, surfactants, hydrophilic compounds, preservatives, anti-oxidants, scavengers, chelating agents, and additional stabilizers. In some embodiments, the composition is water based. In one or more embodiments, the composition has an Aw value of less than 0.9. In some embodiments, the composition has an Aw value of less than 0.8. In some embodiments, the composition has an Aw value of less than 0.7. In some embodiments, the composition has an Aw value of less than 0.6. In some embodiments, the composition has an Aw value of less than 0.5. In some embodiments, the composition has an Aw value of less than 0.4. In some embodiments, the composition has an Aw value of less than 0.3.

In some embodiments, at least 90% by mass of BETi or salt thereof is present in the composition when stored for 3 months at 25° C. In some embodiments, at least about 90% by mass of the BETi or salt thereof is present in the composition when stored for 6 months at 25° C. In some embodiments, at least about 95% by mass of the BETi or salt thereof is present in the composition when stored for 3 months at 25° C. In some embodiments, at least about 95% by mass of the BETi or salt thereof is present in the composition when stored for 6 months at 25° C. In some embodiments, at least about 98% by mass of the BETi or salt thereof is present in the composition when stored for 3 months at 25° C. In some embodiments, at least about 98% by mass of the BETi or salt thereof is present in the composition when stored for 6 months at 25° C. In some embodiments, at least about 99% by mass of the BETi or salt thereof is present in the composition when stored for 3 months at 25° C. In some embodiments, at least about 99% by mass of the BETi or salt thereof is present in the composition when stored for 6 months at 25° C. In some embodiments, the composition is stored at 40° C., and BETi is chemically stable during the aforesaid periods.

In some embodiments, the carrier is about 90% to about 99% by weight of the composition. In some embodiments, the carrier is about 96% to about 99% by weight of the composition. In some embodiments, the solvent and/or emollient is about 90% to about 99% by weight of the composition. In some embodiments, the solvent and/or emollient is about 90% to about 50% by weight of the composition. In some embodiments, the solvent and/or emollient is about 50% to about 16% by weight of the composition. In some embodiments, the solvent and/or emollient is about 10% to about 16% by weight of the composition. In some embodiments, the solvent and/or emollient is about 12% by weight of the composition.

In some embodiments, the BETi at about 0.01% to about 0.7%, or about 0.001%, or about 0.01% or about 0.1% by weight of the composition and the carrier comprises petrolatum and is about 83% to about 99% by weight of the composition and the emollient is about 10% to about 16% by weight of the composition. In some embodiments, the carrier comprises a petrolatum and is about 86% to about 88% by weight of the composition, and the emollient is about 11% to about 14% by weight of the composition. In some embodiments, the carrier comprises about 99% petrolatum. In some embodiments, the carrier consists of a petrolatum.

In some embodiments, the emollient comprises a triglyceride oil comprising a mineral oil, an MCT oil, an olive oil, a coconut oil, a palm oil, a sunflower oil, a rapeseed oil, a soybean oil, a groundnut oil, a peanut oil, a corn oil, a walnut oil, a soya oil, a fish oil, a tallow, a fraction of any of the aforesaid, and mixtures of any two or more thereof.

In one or more embodiments, the BETi is the sole active agent in the composition.

In some embodiments, the carrier or composition is, at room temperature, a semi-solid and, in other embodiments, is a liquid. In some embodiments, the carrier or composition is, at room temperature, a powder or particulate solid, in other embodiments, is a liquid and, yet, in other embodiments, it is a mist.

In some embodiments, the carrier or composition is foamable. In some embodiments, the carrier or composition comprises a foam adjuvant. In some embodiments the carrier or composition is not foamable. Oils are defoamers and silicone oils can be good defoamers. In one or more embodiments it is challenging to achieve a foamable carrier or composition based on silicone oils and other oils that can produce a foam. In some embodiments a foamable composition comprises a reduced amount of elastomer and/or silicone oil and an increased amount of foam adjuvants and surfactants and other hydrophobic solvents. In one or more embodiments, the surfactants are a combination of surfactants forming a complex emulgator and/or having a difference in HLB values of at least 2, or at least 3. In one or more embodiments, polymeric agents which have surfactant properties are used such as poloxamers. In one or more embodiments, the surfactants are silicone surfactants. In one or more embodiments, the formulation is filled in an aerosol cannister to which propellant is added. In one or more embodiments, the formulation is adjusted to reduce the amounts of suspended solids that can potentially block the aerosol cannister valve and to improve the shakability of the canister contents including propellant to a level that will allow repeated use of the cannister without resulting in a block.

In some embodiments, the composition comprises a surfactant. In some embodiments, the surfactants are a combination of surfactants forming a complex emulgator and/or having a difference in HLB values of at least 2, or at least 3. In some embodiments, polymeric agents which have surfactant properties are used, such as poloxamers. In some embodiments, the surfactants are silicone surfactants. In some embodiments the surfactants are polysorbates, such as polyoxyethylene (20) sorbitan monolaurate (polysorbate 20), polyoxyethylene (20) sorbitan monopalmitate (polysorbate 40), polyoxyethylene (20) sorbitan monostearate (polysorbate 60) and polyoxyethylene (20) sorbitan monooleate (polysorbate 80).

In some embodiments, the carrier or composition comprises a propellant. In some embodiments, the propellant is a hydrophobic propellant. In some embodiments, the propellant is a hydrophilic propellant. In some embodiments, the propellant is a liquified or pressurized gas hydrophobic propellant. In some embodiments, the propellant includes one or more of propane, butane and isobutane. In some embodiments, the propellant is AP46, and in others, AP70. In some embodiments, e.g., for topical application to the skin or mucosa as a foam, the propellant is about 3% to about 25%, or about 5% to about 18%, or about 6% to about 15% by weight of the composition. In some embodiments, the ratio of propellant to composition is about 3:100 to about 25:100, or about 5:100 to about 18:100, or about 6:100 to about 15:100 by weight of the composition. In some embodiments, the foamable composition, upon release from a pressurized canister, forms a foam, or, alternatively, a spray. In some embodiments, the foam is quick breaking. In some embodiments, the foam is a breakable foam. In some embodiments, the foam is thermolabile. In some embodiments, the foam is not thermolabile at 37° C. In some embodiments, it has a collapse time at 37° C. of at least about 30 secs, or at least about 60 secs, at least about 90 secs, of at least about 120 secs, or at least about 150 secs, at least about 180 secs, or at least about 240 secs, at least about 300 secs.

In one or more embodiments, the composition, when applied to a surface, does not run. In some embodiments, the composition is not a liquid. In some embodiments, the composition is not a runny liquid. In some embodiments, the composition is thixotropic. In some embodiments, it is shear thinning. By shear thinning is meant that, on the application of stress such as extruding or squeezing through a restricted opening, the composition will act as a lower viscosity composition. So, by way of example, upon application of a shear force to a gel composition, the composition may shear thin and become flowable or fluid.

In one or more embodiments, the carrier or composition, when applied to a skin or mucosal surface, has a bioadhesive or mucoadhesive quality. In one or more embodiments, the carrier facilitates the absorption of BETi such as BETi1 into an epidermal and dermal layer of skin or into the parenchyma or alveoli of the lung. In some embodiments, the carrier facilitates the absorption of BETi, such as BETi1, into mucosal or epithelial layer of the lung. In one or more embodiments, the carrier facilitates the absorption of BETi, such as BETi1, into a mucosal membrane. In one or more embodiments, the carrier facilitates the absorption of the BETi, such as BETi1, into the lining of a body cavity.

In one or more embodiments, delivery of BETi salt (e.g., in the skin, in the parenchyma or alveoli of the lung or mucosal and/or body cavity lining) is higher than with a BETi base. In one or more embodiments, delivery of BETi base (e.g., in the skin, in the parenchyma or alveoli of the lung or mucosal and body cavity lining) is higher than with a BETi salt. In one or more embodiments, delivery of a BETi salt (e.g., through the skin, the parenchyma or alveoli of the lung or mucosal or body cavity lining) is comparable with or lower than with a BETi base. In one or more embodiments, the carrier acts synergistically to enhance delivery even though the BETi, such as BETi1, is not soluble or substantially not soluble in the carrier and emollient. In one or more embodiments delivery may be enhanced where the BETi has a higher solubility in the tissue to where it is applied (e.g., synovial fluid) than in the formulation.

In some embodiments, the carrier comprises petrolatum or a hydrophobic solvent, or mixtures of any two or more thereof. In one or more embodiments the carrier composition further comprises a fragrance agent, a masking agent, a buffering agent, a pH agent, a preservative, a chelating agent, an antioxidant, a scavenger agent, a thickener, a diluent, an additional stabilizer and any mixtures of two or more thereof. In some embodiments, the carrier or composition further comprising at least one of a preservative, a chelating agent, an antioxidant, a scavenger agent, and any mixtures of two or more thereof. In other embodiments, the composition is free or substantially free of a preservative, a chelating agent, an antioxidant, a scavenger agent, and any mixtures of two or more thereof.

In one or more embodiments, there is provided a kit comprising a carrier/composition in a container and a disposable applicator connectable to the container. In some embodiments, the container is a tube. In some embodiments, it is a bottle with a pump. In some embodiments, it is an aerosol canister. In some embodiments, the container is a vial or a syringe. In some embodiments, the container is a capsule. In some embodiments, the container comprises a unit dose means suitable for delivery of a measured unit dose. In some embodiments, the unit dose is about 0.01 mg, or about 0.02 mg, or about 0.03 mg, or about 0.04 mg, or about 0.05 mg, or about 0.06 mg, or about 0.07 mg, or about 0.08 mg, or about 0.09 mg, about 0.1 mg, or about 0.2 mg, or about 0.3 mg, or about 0.4 mg, or about 0.5 mg, or about 0.6 mg, or about 0.7 mg, or about 0.8 mg, or about 0.9 mg, or about 1.0 mg, or about 2 mg, or about 3 mg, or about 4 mg, or about 5 mg, or about 6 mg, or about 7 mg, or about 8 mg, or about 9 mg. In some embodiments, the unit dose is about 0.1 g, or about 0.2 g, or about 0.3 g, or about 0.4 g, or about 0.5 g, or about 0.6 g, or about 0.7 g, or about 0.8 g, or about 0.9 g, or about 1.0 g. In some embodiments, the unit dose is about 0.01 g, or about 0.02 g, or about 0.03 g, or about 0.04 g, or about 0.05 g, or about 0.06 g, or about 0.07 g, or about 0.08 g, or about 0.09 g, or about 1.0 g, or about 2 g, or about 3 g, or about 4 g, or about 5 g, or about 6 g, or about 7 g, or about 8 g, or about 9 g. In some embodiments, the disposable applicator is adapted for delivery of the composition to a body cavity. In some embodiments, the disposable applicator is adapted for delivery of the composition to a close to a joint or joint cavity or space. In some embodiments, the disposable applicator is adapted for delivery of the composition to a body cavity. In some embodiments, the disposable applicator is adapted for delivery of the composition to a skin surface. In some embodiments, the disposable applicator is adapted for delivery of the composition to a mucosal surface.

In some embodiments, there is provided a method of treating a respiratory or lung disorder or disease comprising applying to the airways and lungs (e.g., to lung parenchyma or alveoli) of a subject a BETi composition described herein.

In one or more embodiments, there is provided a method of treating a skin disorder comprising applying to the skin of a subject a BETi composition described herein. In one or more embodiments, there is provided a method of treating a mucosal disorder comprising applying to the mucosa of a subject a BETi composition described herein. In one or more embodiments, there is provided a method of treating a body cavity disorder comprising applying to the body cavity/body cavity surface (e.g., lung) of a subject a composition described herein.

In one or more embodiments, the method involves treating or preventing BET protein responsive dermatoses, e.g., a BDI and/or BDII responsive dermatoses. In one or more embodiments, the composition used in the method includes a BDII and/or a BD1 BET inhibitor, such as BETi1.

In one or more embodiments the skin disorder includes PG, PPP, or GPP

In some embodiments, the disorder is PG. In some embodiments, the disorder is PPP. In some embodiments, the disorder is GPP.

In one or more embodiments, the disorder is a skin disorder.

In some embodiments, the disorder is a fibrosis disorder.

In one or more embodiments, the disorder is a mucosal disorder.

In one or more embodiments, the disorder is a body cavity disorder.

In some embodiments, there is provided a method of treating a joint or joint related disease or disorder comprising administering to a subject a composition described herein.

In some embodiments, the joint or related disorder or disease is chosen from arthritis, bursitis, Ehlers-Danlos syndrome, epicondylitis, Felty Syndrome, gouty arthritis, psoriatic arthritis, osteoarthritis, rheumatoid arthritis, Stills disease, tenosynovitis, synovitis, Sjögren's Syndrome, lyme disease, Whipple disease, bone cancer, lupus, and other autoimmune joint disorders.

In some embodiments, the disease or disorder is rheumatoid arthritis.

In one or more embodiments, there is provided a method of treating or preventing a dermatological disorder or a deterioration thereof comprising applying to the skin of a subject topical composition comprising a BET inhibitor and a carrier in which the BET inhibitor is suspended or substantially suspended. In some embodiments, there is provided a method of treating or preventing a dermatological disorder or a deterioration thereof comprising applying to the body cavity/body cavity surface (e.g., lung) of a subject topical composition comprising a BET inhibitor and a carrier in which the BET inhibitor is dissolved or substantially dissolved. In some embodiments, there is provided a method of treating or preventing a disorder or a deterioration thereof comprising administering to a subject a composition comprising a BETi and a carrier in which the BETi is suspended or substantially suspended or partially suspended. In some embodiments, the composition is administered locally, such as to a joint. In some embodiments, the composition is administered inter-articular to a joint.

In one or more embodiments, the composition is applied to the area of the disorder. In some embodiments, the composition is applied to the area surrounding the area of the disorder. In some embodiments, the composition is applied to the area of the disorder and the area surrounding the disorder.

In one or more embodiments the disease or disorder is an inflammatory or autoimmune disorder comprising a psoriasis. In one or more embodiments, the composition comprises a BET inhibitor, e.g., BETi1, and following seven consecutive days of treatment, the mean psoriasis index is reduced significantly. In some embodiments, following seven consecutive days of treatment the mean psoriasis index is less than 5.5. In some embodiments, following seven consecutive days of treatment the mean psoriasis index is about 5.5. In some embodiments, following seven consecutive days of treatment the mean psoriasis index is less than 4.5. In some embodiments, following seven consecutive days of treatment the mean psoriasis index is about 4.5. In some embodiments, following seven consecutive days of treatment the mean psoriasis index is less than 3.5. In some embodiments, following seven consecutive days of treatment the mean psoriasis index is about 3.5. In some embodiments, following seven consecutive days of treatment the mean psoriasis index is less than 3. In some embodiments, following seven consecutive days of treatment the mean psoriasis index is about three. In some embodiments, following treatment the mean psoriasis index is less than 2.5. In some embodiments, following treatment the mean psoriasis is about 2.5. In some embodiments, following treatment the mean psoriasis index is less than 2. In some embodiments, following treatment the mean psoriasis is about 2. In some embodiments, following treatment the mean psoriasis index is less than 1.5. In some embodiments, following treatment the mean psoriasis is about 1.5. In some embodiments, following treatment the mean psoriasis index is less than 1.25. In some embodiments, following treatment the mean psoriasis is about 1.25. In some embodiments, following treatment the mean psoriasis index is less than 1. In some embodiments, following treatment the mean psoriasis is about 1. In some embodiments, following treatment the mean psoriasis index is less than 0.5. In some embodiments, following treatment the mean psoriasis is about 0.25. In some embodiments, following treatment the mean psoriasis index is less than 0.25. In some embodiments, following treatment the mean psoriasis is about 0.25. In some embodiments wherein the carrier is free or substantially free of one or more of water, surfactants, hydrophilic compounds, preservatives, anti-oxidants, scavengers, chelating agents and additional stabilizers, following treatment the index is less than three. In some embodiments, following treatment the index is about 1.25.

In one or more embodiments, the topical composition comprises a BET inhibitor, e.g., BETi1, which binds efficiently to skin receptors at about 0.001% or less than about 0.001%. In some embodiments, the composition comprises a BETi, e.g., BETi1, which can bind to receptors at about from 0.01% or less than about 0.01%. In some embodiments, the composition comprises a BETi, e.g., BETi1, which binds efficiently to receptors at about from 0.04% or less than about 0.04%. In some embodiments, a dose-response is observed for the BETi-compositions, between the range of about 0.01% and about 10%, or of about 0.04% and about 10%, or of about 0.01% and about 5%, or of about 0.01% and about 1% or of about 0.04% and about 5%, or of about 0.04% and about 1% or of about 0.04% and about 0.4% or about 0.1% and about 1% BETi by weight of composition. In one or more embodiments a dose-response is observed for the BETi-compositions, between the range of about 0.001% and about 1%, or of about 0.001% and about 0.1%, or of about 0.01% and about 1%, or of about 0.01% and about 0.1%. In one or more embodiments, the lowest PASI score is achieved wherein the concentration of BETi is about 0.1%. In one or more embodiments, the lowest PASI score is achieved wherein the concentration of BETi is more than about 0.1%. In one or more embodiments, the lowest PASI score is achieved wherein the concentration of BETi is between about 0.1% to about 1%. In some embodiments, following seven-day treatment with a composition comprising BETi at a concentration of 0.1% the mean psoriasis index is 1.25. In some embodiments, following seven-day treatment the mean psoriasis is about 1.25. In one or more embodiments the mean PASI score value for animals treated with placebo is higher than the mean PASI score values for animals treated with BETi formulations or steroid cream. In one or more embodiments treatment with topical BETi does not result in weight loss and skin thinning. In one or more embodiments treatment with topical BETi is well tolerated. In one or more embodiments treatment with topical BETi at concentrations between 0.1% and 1% has comparable efficacy to a steroid while avoiding steroid untoward side effects such as weight loss and skin thinning. In one or more embodiments the PASI decreasing effect of BETi reaches a plateau when the concentration of the BETi is increased to 0.1%. In one or more embodiments the PASI decreasing effect of BETi reaches a plateau when the concentration of the BETi is increased to 0.2%. In one or more embodiments the PASI decreasing effect of BETi reaches a plateau when the concentration of the BETi is increased to 0.3%. In one or more embodiments the PASI decreasing effect of BETi reaches a plateau when the concentration of the BETi is increased to 0.4%. In one or more embodiments the PASI decreasing effect of BETi reaches a plateau when the concentration of the BETi is increased to 0.5%. In one or more embodiments the PASI decreasing effect of BETi reaches a plateau when the concentration of the BETi is increased to 0.6%. In one or more embodiments the PASI decreasing effect of BETi reaches a plateau when the concentration of the BETi is increased to 0.7%. In one or more embodiments the PASI decreasing effect of BETi reaches a plateau when the concentration of the BETi is increased to 0.8%. In one or more embodiments the PASI decreasing effect of BETi reaches a plateau when the concentration of the BETi is increased to 0.9%. In one or more embodiments the PASI decreasing effect of BETi reaches a plateau when the concentration of the BETi is increased to 1%. In one or more embodiments the PASI decreasing effect of BETi does not reach a plateau when the concentration of the BETi is increased to 1.0%. In one or more embodiments treatment with topical 1% BETi results in a decrease in the mean PASI value score. In one or more embodiments treatment with topical 1% BETi results in about the same mean PASI value score as the 0.001% strength. In one or more embodiments treatment with topical 1% BETi results in a lower mean PASI value score than the 0.001% strength. In one or more embodiments treatment with topical 1% BETi results in a higher mean PASI value score than the 0.001% strength.

In some embodiments, the topical composition comprises a BET inhibitor, e.g., BETi1, which binds efficiently to respiratory system tissue receptors at about from 0.001% or less than about 0.001% or from about 0.06 mg/mL or less than about 0.06 mg/mL. In some embodiments a dose-variable response is observed for the BETi-compositions, between the range of about 0.001% and about 1%, or of about 0.001% and about 0.1%, or of about 0.002% and about 0.08%, or of about 0.01% and about 1%, or of about 0.01% and about 0.1% or between the ranges of about 0.01 mg/mL to about 0.9 mg/mL, of about 0.02 mg/mL to about 0.8 mg/mL, of about 0.03 mg/mL to about 0.7 mg/mL, of about 0.04 mg/mL to about 0.6 mg/mL, of about 0.06 mg/mL to about 6 mg/mL, or about 0.06 mg/mL to about 3 mg/mL, or about 0.6 mg/mL to about 6 mg/mL, or about 0.6 mg/mL to about 3 mg/mL.

It should be noted that compositions disclosed herein, e.g., topical compositions, can be applied to the target site as a gel or a semi-solid gel. In certain other embodiments, it can be applied as an ointment, or a liquid, or a foam or a breakable foam.

Application of the claimed compositions can be, for example, hourly, twelve hourly (e.g., twice daily), daily, alternate-day or intermittent, according to the condition of the patient and the type and stage or state of advancement of the disease or disorder. For reasons of compliance, less frequent applications, where possible, are preferable, e.g., daily, or once per two days or three days, or weekly, monthly, or quarterly single applications. Such longer periods may be appropriate where the composition is formulated for extended release. In certain cases, for example where prolonged or long-term treatment is desired or required, an initial dose is provided, followed by a gradual reduction to a lower maintenance dose, which can be increased if further outbreaks occur or according to the condition of the patient. In certain cases, for example where prolonged or long-term treatment is desired or required, in one or more embodiments an initial dose is provided, followed by a further dose after a week or a month, which may be followed by an additional dose, after a month, three month or six months.

In one or more embodiments, the carrier comprises at least one hydrophobic agent. In one or more embodiments, the hydrophobic agent or at least one hydrophobic agent comprises or is selected from the group consisting of an oil, a mineral oil, a hydrocarbon oil, an ester oil, an ester of a dicarboxylic acid, a triglyceride oil, an oil of plant origin, an oil from animal origin, an unsaturated or polyunsaturated oil, a diglyceride, a PPG alkyl ether, an essential oil, a silicone oil, a liquid paraffin, an isoparaffin, a polyalphaolefin, a polyolefin, a polyisobutylene, a synthetic isoalkane, isohexadecane, isododecane, alkyl benzoate, alkyl octanoate, C₁₂-C₁₅ alkyl benzoate, C₁₂-C₁₅ alkyl octanoate, arachidyl behenate, arachidyl propionate, benzyl laurate, benzyl myristate, benzyl palmitate, bis(octyldodecyl stearoyl) dimer dilinoleate, butyl myristate, butyl stearate, cetearyl ethylhexanoate, cetearyl isononanoate, cetyl acetate, cetyl ethylhexanoate, cetyl lactate, cetyl myristate, cetyl octanoate, cetyl palmitate, cetyl ricinoleate, decyl oleate, diethyleneglycol diethylhexanoate, diethyleneglycol dioctanoate, diethyleneglycol diisononanoate, diethyleneglycol diisononanoate, diethylhexanoate, diethylhexyl adipate, diethylhexyl malate, diethylhexyl succinate, diisopropyl adipate, diisopropyl dimerate, diisopropyl sebacate, diisosteary dimer dilinoleate, diisostearyl fumerate, dioctyl malate, dioctyl sebacate, dodecyl oleate, ethylhexyl palmitate, ester derivatives of lanolic acid, ethylhexyl cocoate, ethylhexyl ethylhexanoate, ethylhexyl hydroxystarate, ethylhexyl isononanoate, ethylhexyl palmytate, ethylhexyl pelargonate, ethylhexyl stearate, hexadecyl stearate, hexyl laurate, isoamyl laurate, isocetyl behenate, isocetyl lanolate, isocetyl palmitate, isocetyl stearate, isocetyl salicylate, isocetyl stearate, isocetyl stearoyl stearate, isocetearyl octanoate, isodecyl ethylhexanoate, isodecyl isononanoate, isodecyl oleate, isononyl isononanoate, isodecyl oleate, isohexyl decanoate, isononyl octanoate, isopropyl isostearate, isopropyl lanolate, isopropyl laurate, isopropyl myristate, isopropyl palmitate, isopropyl stearate, isostearyl behenate, isosteary citrate, isostearyl erucate, isostearyl glycolate, isostearyl isononanoate, isostearyl isostearate, isostearyl lactate, isostearyl linoleate, isostearyl linolenate, isostearyl malate, isostearyl neopentanoate, isostearyl palmitate, isosteary salicylate, isosteary tartarate, isotridecyl isononanoate, isotridecyl isononanoate, lauryl lactate, myristyl lactate, myristyl myristate, myristyl neopentanoate, myristyl propionate, octyldodecyl myristate, neopentylglycol dicaprate, octyl dodecanol, octyl stearate, octyl palmitate, octyldodecyl behenate, octyldodecyl hydroxystearate, octyldodecyl myristate, octyldodecyl stearoyl stearate, oleyl erucate, oleyl lactate, oleyl oleate, propyl myristate, propylene glycol myristyl ether acetate, propylene glycol dicaprate, propylene glycol dicaprylate, propylene glycol dicaprylate, maleated soybean oil, stearyl caprate, stearyl heptanoate, stearyl propionate, tocopheryl acetate, tocopheryl linoleate, glyceryl oleate, tridecyl ethylhexanoate, tridecyl isononanoate, triisocetyl citrate, alexandria laurel tree oil, an avocado oil, an apricot stone oil, a barley oil, a borage seed oil, a calendula oil, a canelle nut tree oil, a canola oil, a caprylic/capric a triglyceride castor oil, a coconut oil, a corn oil, a cotton oil, a cottonseed oil, an evening primrose oil, a flaxseed oil, a groundnut oil, a hazelnut oil, glycereth triacetate, glycerol triheptanoate, glyceryl trioctanoate, glyceryl triundecanoate, a hempseed oil, a jojoba oil, a lucerne oil, a maize germ oil, a marrow oil, a millet oil, a neopentylglycol dicaprylate/dicaprate, an olive oil, a palm oil, a passionflower oil, pentaerythrityl tetrastearate, a poppy oil, propylene glycol ricinoleate, a rapeseed oil, a rye oil, a safflower oil, a sesame oil, a shea butter, a soya oil, a soybean oil, a sweet almond oil, a sunflower oil, a sysymbrium oil, a syzigium aromaticum oil, a tea tree oil, a walnut oil, wheat germ glycerides, a wheat germ oil, PPG-2 butyl ether, PPG-4 butyl ether, PPG-5 butyl ether, PPG-9 butyl ether, PPG-12 butyl ether, PPG-14 butyl ether, PPG-15 butyl ether, PPG-15 stearyl ether, PPG-16 butyl ether, PPG-17 butyl ether, PPG-18 butyl ether, PPG-20 butyl ether, PPG-22 butyl ether, PPG-24 butyl ether, PPG-26 butyl ether, PPG-30 butyl ether, PPG-33 butyl ether, PPG-40 butyl ether, PPG-52 butyl ether, PPG-53 butyl ether, PPG-10 cetyl ether, PPG-28 cetyl ether, PPG-30 cetyl ether, PPG-50 cetyl ether, PPG-30 isocetyl ether, PPG-4 lauryl ether, PPG-7 lauryl ether, PPG-2 methyl ether, PPG-3 methyl ether, PPG-3 myristyl ether, PPG-4 myristyl ether, PPG-10 oleyl ether, PPG-20 oleyl ether, PPG-23 oleyl ether, PPG-30 oleyl ether, PPG-37 oleyl ether, PPG-40 butyl ether, PPG-50 oleyl ether, PPG-11 stearyl ether, a herring oil, a cod-liver oil, a salmon oil, a cyclomethicone, a dimethyl polysiloxane, a dimethicone, an epoxy-modified silicone oil, a fatty acid-modified silicone oil, a fluoro group-modified silicone oil, a methylphenylpolysiloxane, phenyl trimethicone, a polyether group-modified silicone oil and mixtures of any two or more thereof. In some embodiments, the hydrophobic agent comprises or is selected from the group consisting of a soybean oil, a coconut oil, a cyclomethicone, a light mineral oil, a heavy mineral oil and mixtures thereof. In one or more embodiments, the solvent is tested individually for compatibility with BETi and is only used if it passes a compatibility test.

In one or more embodiments, the hydrophobic agent is at a concentration of about 75% to about 99.999% by weight. In one or more embodiments, the hydrophobic agent is at a concentration of at least about 40% by weight, at least about 45% by weight, at least about 50% by weight, at least about 55% by weight, at least about 60% by weight, at least about 65% by weight, at least about 70% by weight, at least about 75% by weight, at least about 80% by weight, at least about 85% by weight, at least about 90% by weight at least about 92% by weight, at least about 95% by weight or at least about 99.999% by weight and any ranges between any two figures listed for example from about 55% to about 99%. In some embodiments, the hydrophobic agent is at a concentration of less than about 90% by weight, less than about 80% by weight, less than about 70% by weight, less than about 60% by weight, less than about 50% by weight. In one or more embodiments, the hydrophobic agent is at a concentration of about 70% by weight, or about 72% by weight, or about 74% by weight, or about 76% by weight, or about 78% by weight, or about 80% by weight, or about 82% by weight, or about 84% by weight, or about 86% by weight, or about 88% by weight, or about 90% by weight, or about 92% by weight, or about 94% by weight, or about 96% by weight, or about 98% by weight. In one or more embodiments, the hydrophobic agent is petrolatum.

In some embodiments, the carrier comprises at least one hydrophilic agent.

In some embodiments, the solvent is tested individually for compatibility with BETi and is only used if it passes a compatibility test.

In some embodiments, the at least one hydrophilic agent is at a concentration of about 75% to about 99.999% by weight. In some embodiments, the at least one hydrophilic agent is at a concentration of at least about 40% by weight, at least about 45% by weight, at least about 50% by weight, at least about 55% by weight, at least about 60% by weight, at least about 65% by weight, at least about 70% by weight, at least about 75% by weight, at least about 80% by weight, at least about 85% by weight, at least about 90% by weight at least about 92% by weight, at least about 95% by weight or at least about 99.999% by weight and any ranges between any two figures listed for example from about 55% to about 99%. In some embodiments, the at least one hydrophilic agent is at a concentration of less than about 90% by weight, less than about 80% by weight, less than about 70% by weight, less than about 60% by weight, less than about 50% by weight. In some embodiments, the at least one hydrophilic agent is at a concentration of about 70% by weight, or about 72% by weight, or about 74% by weight, or about 76% by weight, or about 78% by weight, or about 80% by weight, or about 82% by weight, or about 84% by weight, or about 86% by weight, or about 88% by weight, or about 90% by weight, or about 92% by weight, or about 94% by weight, or about 96% by weight, or about 98% by weight.

In one or more embodiments, the emollient comprises or is selected from the group consisting of isostearic acid derivatives, isopropyl palmitate, lanolin oil, diisopropyl dimerate, diisopropyl adipate, dimethyl isosorbide, maleated soybean oil, octyl palmitate, isopropyl isostearate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, hydrogenated coco-glycerides, isononyl isononanoate, isotridecyl isononanoate, myristyl myristate, triisocetyl citrate, octyl dodecanol, octyl hydroxystearate and mixtures thereof. Alternatively, in some embodiments, the emollient comprises or is chosen from a glycol, a polyethylene glycol, a propylene glycol, an alcohol, a short chain alcohol, glycerol, a diol, a triol and a polyol. Other examples of other suitable emollients can also be found in the Cosmetic Bench Reference, pp. 1.19-1.22 (1996), which is incorporated herein by reference for emollients.

In some embodiments, the hydrophilic agent or polar solvent is at a concentration of about 75% to about 99.999% by weight. In some embodiments, the hydrophilic agent or polar solvent is at a concentration of at least about 40% by weight, at least about 45% by weight, at least about 50% by weight, at least about 55% by weight, at least about 60% by weight, at least about 65% by weight, at least about 70% by weight, at least about 75% by weight, at least about 80% by weight, at least about 85% by weight, at least about 90% by weight at least about 92% by weight, at least about 95% by weight or at least about 99.999% by weight and any ranges between any two figures listed for example from about 55% to about 99%. In some embodiments, the hydrophilic agent or polar solvent is at a concentration of less than about 90% by weight, less than about 80% by weight, less than about 70% by weight, less than about 60% by weight, less than about 50% by weight. In some embodiments, the hydrophilic agent or polar solvent is at a concentration of about 70% by weight, or about 72% by weight, or about 74% by weight, or about 76% by weight, or about 78% by weight, or about 80% by weight, or about 82% by weight, or about 84% by weight, or about 86% by weight, or about 88% by weight, or about 90% by weight, or about 92% by weight, or about 94% by weight, or about 96% by weight, or about 98% by weight.

In one or more embodiments, the composition comprises a fatty alcohol and/or a fatty acid. In one or more embodiments, the fatty alcohol and/or fatty acid have a melting point of at least about 40° C.

In one or more embodiments, the fatty alcohol comprises or is selected from the group consisting of decyl alcohol, undecanol, lauryl alcohol, tridecanol, myristyl alcohol, pentadecanol, cetyl alcohol, cis-9-hexadecen-1-ol, heptadecanol, stearyl alcohol, oleyl alcohol, arachidyl alcohol, behenyl alcohol, tetracosanol, hexacosanol, octacosanol, triacontanol, and tetratriacontanol. In one or more embodiments, the fatty acid comprises or is selected from the group consisting of capric acid, dodecanoic acid, tetradecanoic acid, hexadecanoic acid, heptadecanoic acid, octadecanoic acid, arachidic acid, eicosanoic acid, docosanoic acid, tetracosanoic acid, hexacosanoic acid, heptacosanoic acid, octacosanoic acid, triacontanoic acid, dotriacontanoic acid, tritriacontanoic acid, tetratriacontanoic acid, and pentatriacontanoic acid.

In some embodiments, the fatty alcohol or the fatty acid is about 0.1% to about 1% by weight. In some embodiments, the fatty alcohol or the fatty acid is about 1% to about 3% by weight. In one or more embodiments, the fatty alcohol or the fatty acid is about 3% to about 10% by weight. For example, about 3% by weight, or about 4% by weight, or about 5% by weight, or about 6% by weight, or about 7% by weight, or about 8% by weight, or about 9% by weight, or about 10% by weight. For example, about 4.1% by weight, or about 4.4% by weight, or about 4.5% by weight, or about 5% by weight, or about 5.6% by weight, or about 8.6% by weight.

In one or more embodiments, the fatty alcohol is less than about 8% by weight. For example, less than about 7% by weight, or less than about 6% by weight, or less than about 5% by weight, or less than about 4% by weight.

In one or more embodiments, the carbon chain of the fatty alcohol or the fatty acid is substituted with a hydroxyl group.

In one or more embodiments, the fatty acid is 12-hydroxy stearic acid.

In one or more embodiments the composition comprises a modifying agent. In one or more embodiments, the modifying agent is a wax comprising or selected from the group consisting of a plant wax, carnauba wax, candelilla wax, ouricury wax, sugarcane wax, retamo wax, jojoba oil, an animal waxes, beeswax, a petroleum derived wax, a paraffin wax, polyethylene, and derivatives thereof.

In one or more embodiments, the modifying agent is a combination comprising (i) at least one fatty alcohol and at least one fatty acid; or (ii) at least one fatty alcohol and at least one wax; or (iii) at least one fatty acid and at least one wax; or (iv) at least one fatty alcohol, at least one fatty acid, and at least one wax.

In one or more embodiments, the at least one modifying agent comprises or is selected from the group consisting of a fatty alcohol, a fatty acid and a wax, wherein the fatty alcohols and/or fatty acids have at least 12 carbon atoms in their carbon backbone. In certain embodiments the modifying agent is a combination of a fatty alcohol and a fatty acid and/or a wax.

In some embodiments, the fatty alcohol and/or fatty acid and/or wax are solid at ambient temperature. In certain embodiments, the fatty alcohol and/or the fatty acid and/or the wax or the mixture of them have a melting point of more than about 40° C.

In some embodiments, the wax is about 0% to about 0.6% by weight. For example, about 0.1% by weight, or about 0.2% by weight, or about 0.3% by weight, or about 0.4% by weight, or about 0.5% by weight, or about 0.6% by weight. In some embodiments, the wax is less than about 0.1% by weight. In one or more embodiments, the wax is about 0% to about 6% by weight. For example, about 1% by weight, or about 2% by weight, or about 3% by weight, or about 4% by weight, or about 5% by weight, or about 6% by weight. In one or more embodiments, the wax is about 0.2% by weight.

In one or more embodiments, the wax is less than about 4% by weight. For example, less than about 3% by weight, or less than about 2% by weight, or less than about 1% by weight, or less than about 0.5% by weight.

In one or more embodiments, the fatty acid is about 1% to about 10% by weight. For example, about 1% by weight, or about 2% by weight, or about 3% by weight, or about 4% by weight, or about 5% by weight, or about 6% by weight, or about 7% by weight, or about 8% by weight, or about 9% by weight, or about 10% by weight. For example, about 2.4% by weight, or about 2.5% by weight, or about 3% by weight.

In one or more embodiments, the total amount of fatty acid fatty alcohol and wax, if present is about 1% to about 10% by weight. For example, about 1% by weight, or about 2% by weight, or about 3% by weight, or about 4% by weight, or about 5% by weight, or about 6% by weight, or about 7% by weight, or about 8% by weight, or about 9% by weight, or about 10% by weight. For example, about 2.4% by weight, or about 2.5% by weight, or about 3% by weight.

In some embodiments, the composition comprises a mucoadhesive or bioadhesive agent or excipient. Mucoadhesive/bioadhesion has been defined as the attachment of synthetic or biological macromolecules to a biological tissue. Mucoadhesive agents are a class of polymeric biomaterials that exhibit the basic characteristic of a hydrogel, i.e., swell by absorbing water and interacting by means of adhesion with the mucous that covers epithelia. Compositions of the present invention may contain a mucoadhesive macromolecule or polymer in an amount sufficient to confer bioadhesive properties. The bioadhesive macromolecule enhances the delivery of biologically active agents on or through the target surface. The mucoadhesive macromolecule may be selected from acidic synthetic polymers, preferably having an acidic group per four repeating or monomeric subunit moieties, such as poly(acrylic)- and/or poly(methacrylic) acid (e.g., Carbopol®, Carbomer®), poly(methylvinyl ether/maleic anhydride) copolymer, and their mixtures and copolymers; acidic synthetically modified natural polymers, such as carboxymethylcellulose (CMC); neutral synthetically modified natural polymers, such as (hydroxypropyl)methylcellulose; basic amine-bearing polymers such as chitosan; acidic polymers obtainable from natural sources, such as alginic acid, hyaluronic acid, pectin, gum tragacanth, and karaya gum; and neutral synthetic polymers, such as polyvinyl alcohol or their mixtures. An additional group of mucoadhesive polymers includes natural and chemically modified cyclodextrin, especially hydroxypropyl-β-cyclodextrin. Such polymers may be present as free acids, bases, or salts. Many mucoadhesive agents are known in the art to also possess gelling properties.

In one or more embodiments, the polymeric agent contains a film-forming component. The film-forming component may include a water-insoluble alkyl cellulose or hydroxyalkyl cellulose. Exemplary alkyl cellulose or hydroxyalkyl cellulose polymers include ethyl cellulose, propyl cellulose, butyl cellulose, cellulose acetate, hydroxypropyl cellulose, hydroxybutyl cellulose, and ethylhydroxyethyl cellulose, alone or in combination. In addition, a plasticizer or a cross-linking agent may be used to modify the polymer's characteristics. For example, esters such as dibutyl or diethyl phthalate, amides such as diethyldiphenyl urea, vegetable oils, fatty acids and alcohols such as oleic and myristyl acid may be used in combination with the cellulose derivative.

In one or more embodiments, the polymeric agent includes a phase change polymer, which alters the composition behavior from fluid-like prior to administration to solid-like upon contact with the target mucosal surface. Such phase change results from external stimuli, such as changes in temperature or pH and exposure to specific ions (e.g., Ca2+). Non-limiting examples of phase change polymers include poly(N-isopropylamide) and Poloxamer 407®.

In one or more embodiments, the composition comprises a silicone-based polymer. In one or more embodiments, non-limiting examples include dimethicone crosspolymer, polysilicone-11, polymethylsilsesquioxane and mixtures thereof.

In one or more embodiments, the composition comprises a polymer selected from the group including ethylene/propylene/styrene copolymer, butylene/ethylene/styrene copolymer, butylated hydroxyl toluene or similar gelling agents.

In some embodiments, the polymers may be cured ex-vivo forming a solid matrix that incorporates the drug for controlled release to an inflammatory region. Suitable biodegradable polymers may be natural or synthetic. Natural biocompatible biodegradable polymers include, but are not limited to, proteins, such as albumin, collagen, gelatin, synthetic poly(aminoacids), and prolamines; glycosaminoglycans, such as hyaluronic acid and heparin; and polysaccharides, such as alginates, chitosan, starch, and dextrans. Synthetic biocompatible biodegradable materials include, but are not limited to, poly(lactide-co-glycolide) (PLGA), polylactide (PLA), polyglycolide (PG), polyhydroxybutyric acid, poly(trimethylenecarbonate), polycaprolactone (PCL), polyvalerolactone, poly(alpha-hydroxy acids), poly(lactones), poly(amino-acids), poly(anhydrides), polyketals poly(arylates), poly(orthoesters), polyurethanes, polythioesters, poly(orthocarbonates), poly(phosphoesters), poly(ester-co-amide), poly(lactide-co-urethane, polyethylene glycol (PEG), polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA blends and copolymers thereof and any combinations thereof. The biocompatible biodegradable material can include a combination of biocompatible biodegradable materials. For example, the biocompatible biodegradable material can be a triblock, or other multi-block, formation where a combination of biocompatible biodegradable polymers are joined together.

In one or more embodiments, the composition comprises an anti-oxidant. In one or more embodiments, non-limiting examples are: ascorbic acid/ascorbate, ascorbyl palmitate, butylated hydroxyanisole, butylated hydroxytoluene, citric acid/sodium citrate, disodium EDTA, propyl gallate, sodium metabisulfite sodium sulfite, sodium thiosulfate, tartaric acid/sod. Tartrate, tocopherol, tocophersolan and mixtures thereof.

In one or more embodiments, the modifying agent is a wax comprising or selected from the group consisting of a plant wax, carnauba wax, candelilla wax, ouricury wax, sugarcane wax, retamo wax, jojoba oil, an animal waxes, beeswax, a petroleum derived wax, a paraffin wax, polyethylene, and derivatives thereof.

In one or more embodiments, the modifying agent is a combination comprising (i) at least one fatty alcohol and at least one fatty acid; or (ii) at least one fatty alcohol and at least one wax; or (iii) at least one fatty acid and at least one wax; or (iv) at least one fatty alcohol, at least one fatty acid, and at least one wax.

In one or more embodiments, the at least one modifying agent comprises or is selected from the group consisting of a fatty alcohol, a fatty acid and a wax, wherein the fatty alcohols and/or fatty acids have at least 12 carbon atoms in their carbon backbone. In certain embodiments, the modifying agent is a combination of a fatty alcohol and a fatty acid and/or a wax.

In some embodiments, the fatty alcohol and/or fatty acid and/or wax are solid at ambient temperature. In certain embodiments, the fatty alcohol and/or the fatty acid and/or the wax or the mixture of them have a melting point of more than about 40° C.

In one or more embodiments, the wax is about 0% to about 6% by weight. For example, about 1% by weight, or about 2% by weight, or about 3% by weight, or about 4% by weight, or about 5% by weight, or about 6% by weight. In one or more embodiments, the wax is about 0.2% by weight.

In one or more embodiments, the wax is less than about 4% by weight. For example, less than about 3% by weight, or less than about 2% by weight, or less than about 1% by weight, or less than about 0.5% by weight.

In one or more embodiments, the fatty acid is about 1% to about 10% by weight. For example, about 1% by weight, or about 2% by weight, or about 3% by weight, or about 4% by weight, or about 5% by weight, or about 6% by weight, or about 7% by weight, or about 8% by weight, or about 9% by weight, or about 10% by weight. For example, about 2.4% by weight, or about 2.5% by weight, or about 3% by weight.

In one or more embodiments, the total amount of fatty acid fatty alcohol and wax, if present, is about 1% to about 10% by weight. For example, about 1% by weight, or about 2% by weight, or about 3% by weight, or about 4% by weight, or about 5% by weight, or about 6% by weight, or about 7% by weight, or about 8% by weight, or about 9% by weight, or about 10% by weight. For example, about 2.4% by weight, or about 2.5% by weight, or about 3% by weight.

In one or more embodiments, a topical composition comprising a BETi is effective and well tolerated for treatment of wounds in a mammalian subject.

In one or more embodiments, a topical composition comprising a BETi is effective and well tolerated for treatment of PG, GPP or PPP in a mammalian subject.

In some embodiments, a composition comprising a BETi is effective and well tolerated for treatment of a joint or joint related disease or disorder in a subject. In some embodiments, the composition comprising a BETi (e.g., BETi1) comprises 7.5 mg of BETi/subject dose. In some embodiments, the composition comprising a BETi (e.g., BETi1) comprises 75 mg of BETi/subject dose. In some embodiments, the composition comprising a BETi (e.g., BETi1) comprises 150 mg of BETi/subject dose. In some embodiments, the composition comprises carboxymethylcellulose, polysorbate 80 and/or saline. In some embodiments, the control and BETi (e.g., BETi1) dose formulations comprise:
a. Control: Vehicle (0.5% sodium carboxymethylcellulose; 0.1% polysorbate 80 in saline);
b. Low Dose: 0.75% w/w BETi1 suspended in Vehicle;
c. Mid Dose: 7.5% w/w BETi1 suspended in Vehicle; and
d. High Dose: 15% w/w BETi1 suspended in Vehicle.

The viscosity of a composition is an important consideration when formulating topical drug products, such as semi-solid topical drug products. On the one hand, viscosity should be high enough to enable inter alia: (i) proper dispensing of the product on the patient's skin without having a runny liquid, or alternatively, proper dispensing of the product and functioning within the joint or area of the joint, or, alternatively, proper dispensing of the product on the patient's target area without running off, (ii) an adequate feel, i.e., skin feel, to ensure patient compliance, (iii) if active ingredient is suspended, a uniform distribution of the active ingredient for avoidance of aggregates of API crystals. On the other hand, the viscosity of the drug product should be low enough to enable inter alia: (i) proper extrusion of the product from the container (e.g. tube or pump or vial or syringe), (ii) good feel, i.e., skin feel, for improved patient compliance, or alternatively, good feel and ease of use, (iii) uniform distribution of the composition over the target surface or tissue, (iv) an industrially applicable compounding and packaging manufacturing process.

The manufacture and scale-up of a semi-solid drug product comprising low concentration of active ingredient may also be challenging. When a low concentration of active ingredient is suspended, it may be difficult to obtain a uniform distribution of the active in the bulk product, especially on industrial scale. In addition, chemical stability issues may arise when the concentration of the active ingredient is decreased substantially due to the change in the active to excipient ratio. The viscosity of the formulation during manufacturing, in one or more embodiments, should facilitate homogenous mixing of the ingredients and uniform distribution of suspended matter.

In one or more embodiments, the viscosity is measured by an Anton Par Rheometer MCR302, plate/plate 50 mm geometry (“the Anton Par”). In one or more embodiments, shear force can be measured at different shear rates, e.g., 100 sec⁻¹, 10 sec⁻¹, 1 sec⁻¹, or 0.1 sec⁻¹. In one or more embodiments, the viscosity is measured using a DHR3 rheometer from TA instruments.

In one or more other embodiments, the viscosity is measured by a Brookfield viscometer, such as a DV II CP. As will be appreciated by one skilled in the art, viscosity measurements can vary according, amongst other things, to the viscometer used, the shear rate used, the spindle and the container and the volume of composition.

In one or more embodiments, the viscosity increases when the temperature increases.

In the context herein, a petrolatum-based formulation is one where the majority of the formulation comprises petrolatum.

### The Ingredients as Therapeutic Agents

In one or more embodiments, the excipients in the composition can have a therapeutic effect that completes and/or enhances and/or complements the BET inhibitor effect. In some embodiments, the excipient, when applied together with the active agent(s), can have a synergistic effect. In some embodiments, the excipient, when applied together with the active agent(s), can have a modifying or ameliorating effect, e.g., by gene regulation.

In certain embodiments, a hydrophobic agent or carrier can possess therapeutic properties. For example, some oils, e.g., some essential oils can kill microorganisms or impair their growth and can be effective or supportive in the treatment or prevention of conditions that involve microbial infection, such as bacterial, fungal and viral conditions. Additionally, hydrophobic agents can be useful for the treatment of conditions that involve damaged skin, such as psoriasis or atopic dermatitis. The combination of a hydrophobic agent or carrier and a therapeutically effective fatty alcohol or fatty acid may afford a beneficial effect in conditions characterized, for example, by infection and/or inflammation.

Fatty alcohols can also possess therapeutic properties. Long chain saturated and monounsaturated fatty alcohols, e.g., stearyl alcohol, erucyl alcohol, arachidyl alcohol and behenyl alcohol (docosanol), have been reported to possess antiviral, anti-infective, antiproliferative and anti-inflammatory properties (see, e.g., U.S. Pat. No. 4,874,794). Longer chain fatty alcohols, e.g., tetracosanol, hexacosanol, heptacosanol, octacosanol, triacontanol, etc., are also known for their metabolism-modifying properties, and tissue-energizing properties.

In one or more embodiments, the active agent can be a placebo or a cosmetic agent. In one or more embodiments, the composition is suitable for use in the manufacture of a medicament, including a placebo or active agent.

The stability of compositions containing an active agent, e.g., a BETi, can be monitored at about e.g. 5° C., 25° C., 30° C. and 40° C., and satisfactory stability results are obtained.

In one or more embodiments, there is provided a composition in which the composition comprises an additional agent including one or more of a disinfectant, an alpha hydroxyl acid, lactic acid, glycolic acid, a beta-hydroxy acid, a protein, an amino acid, an oligosaccharide, a haptene, an oxidizing agent, an antioxidant, benzoyl chloride, calcium hypochlorite, magnesium hypochlorite, an anti-wrinkle agent, a radical scavenger, talc, carbon, a skin whitening agent, a skin protective agent, a masking agent, a refatting agent, and a lubricating agent.

In one or more embodiments, the concentration of the additional agent is about any of the amounts or between about one or more of any of the aforesaid ranges for the additional active agent.

In one or more embodiments, a composition containing skin irritants, such as surfactants and short chain alcohols, is disadvantageous, and especially where the composition is applied frequently e.g., daily and/or for prolonged periods and/or the potential irritant is present in higher concentrations even if applied less frequently or for shorter periods. In one or more embodiments, a composition is free of skin irritants, such as surfactants and short chain alcohols. In one or more embodiments, a composition is essentially free of skin irritants such as surfactants and short chain alcohols. In one or more embodiments, a composition is substantially free of skin irritants such as surfactants and short chain alcohols.

The nature of a formulation in general terms is determined by the content of the formulation which can be as a liquid, or semi-solid, or a gel or any other flowable substance, and, for some aerosols and foamable compositions, also by the inclusion of propellant, the type of propellant, and the amount of propellant. If no propellant or less than 3% propellant is included, the formulation is a liquid, or semi-solid, or a gel. If the content includes propellant, say about 3% to about 50%, it can, in some embodiments, emerge as a foam. If the content includes more than 50% of propellant say even up to 95%, it can emerge, in some embodiments, as a spray. In one or more embodiments, e.g., where the propellant is separate from the content, the content may be expelled as a mousse, cream, gel, lotion or any other flowable substance. In one or more embodiments, a spray is disadvantageous. In one or more embodiments, the carrier or composition is not a spray. In one or more embodiments, the propellant is less than 55%, or less than 50%, or less than 45%, or less than 40%, or less than 35% or less than 30%, or less than 20% or less than 10% or less than 5% less than 3% or less than 2% or less than 1%. In one or more embodiments, the formulation is not a foam. In some embodiments, the carrier or composition is not a gel. In one or more embodiments, the carrier or composition is not a liquid. In one or more embodiments, the carrier or composition is a semi-solid. In one or more embodiments, the carrier or composition is an ointment. In one or more embodiments, the carrier or composition is a gel.

In one or more embodiments, the carrier or composition is not hydrophilic or substantially not hydrophilic.

In one or more embodiments, the carrier or composition is a hydrophobic carrier. In one or more embodiments, the hydrophobic carrier is free of or substantially free of hydrophilic compounds. In some embodiments, the carrier or composition is hydrophilic or substantially hydrophilic. In some embodiments, the hydrophilic carrier is free of or substantially free of hydrophobic compounds.

In one or more embodiments, the carrier or composition is free or substantially free of at least of one or more of water, hydrophilic solvents, surface-active agents, protic solvents, polar protic solvents, aprotic solvents, polyols, short chain alcohols, propellant, and aldehyde scavengers. In one or more embodiments, the carrier is essentially free of one or more of the aforesaid. In one or more embodiments, the carrier comprises less than about 0.4%, or less than about 0.3%, or less than about 0.2%, or less than about 0.1%, or less than about 0.05% of one or more of the aforesaid. In some embodiments, the carrier or composition is chosen from water, hydrophilic solvents, surface-active agents, protic solvents, polar protic solvents, aprotic solvents, polyols, short chain alcohols, aldehyde scavengers, and mixtures thereof.

In one or more embodiments, the carrier or composition is free, essentially free or substantially free of aldehyde scavengers comprising glycerine and anti-oxidants. In one or more embodiments, the vehicle is free, essentially free or substantially free, of anti-oxidant e.g., comprising one or more of alpha-tocopherol, butyl hydroxy anisol (BHA), butyl hydroxy toluene (BHT) and propyl gallate. In some embodiments, the carrier or composition comprises aldehyde scavengers comprising glycerine and anti-oxidants. In some embodiments, the vehicle comprises anti-oxidant e.g., comprising one or more of alpha-tocopherol, butyl hydroxy anisol (BHA), butyl hydroxy toluene (BHT), and propyl gallate.

In one or more embodiments, the carrier or composition is free, essentially free or substantially free of one or more of water, ethyl acetate, acetonitrile, IPAc, t-Butyl methyl ether, heptane, dichloromethane or anisole. In some embodiments, the carrier or composition is chosen from water, ethyl acetate, acetonitrile, IPAc, t-Butyl methyl ether, heptane, dichloromethane, anisole, and mixtures thereof.

In one or more embodiments, the carrier or composition is free, essentially free, or substantially free of a solvent which can dissolve BETi, wherein said solvent includes one or more of or is selected from the group consisting of water, ethyl acetate, acetonitrile, IPAc, t-Butyl methyl ether, heptane, dichloromethane or anisole. In some embodiments, the carrier or composition is or comprises a solvent which can dissolve BETi, wherein said solvent includes one or more of or is selected from the group consisting of water, ethyl acetate, acetonitrile, IPAc, t-Butyl methyl ether, heptane, dichloromethane, and anisole.

In one or more embodiments, the solvent comprises or is a combination of tetrahydrofuran, 1,4-Dioxane, methanol, polyethylene glycol 400, acetone, methyl ethyl ketone, ethanol or isopropanol and wherein the solvent less than about 80%, or less than about 75%, or less than about 70%, or less than about 65%, or less than about 60%, or less than about 55%, or less than about 50%, or less than about 45%, or less than about 40%, or less than about 35%, or less than about 30%, or less than about 25%, or less than about 20%, or less than about 15%, or less than about 10% or less than about 7.5% or less than about 5% or less than about 2 or less than about 1% of the composition.

In some embodiments, the amount of a BETi applied topically is about 0.0001% to about 0.1% by weight of the composition. In some embodiments, the amount of a BETi applied topically is about 0.0002% to about 0.1% by weight of the composition. In some embodiments, the amount of a BETi applied topically is about 0.0005% to about 0.05% by weight of the composition. In some embodiments, the amount of a BETi applied topically is about 0.001% to about 0.01% by weight of the composition. In some embodiments, the amount of a BETi applied topically is about 0.001% to about 1% by weight of the composition. In some embodiments, the amount of a BETi applied topically is about 0.002% to about 0.1% by weight of the composition. In some embodiments, the amount of a BETi applied topically is about 0.005% to about 0.01% by weight of the composition. In some embodiments, the amount of a BETi applied topically is about 0.001% to about 0.05% by weight of the composition. In some embodiments, the amount of a BETi applied topically is about 0.0001% to about 10% by weight of the composition. In some embodiments, the amount of a BETi applied topically is about 0.001% to about 5% by weight of the composition. In some embodiments, the amount of a BETi applied topically is above about 0.001% by weight of the composition. In some embodiments, the amount of a BETi applied topically is above about 0.005% by weight of the composition. In some embodiments, the amount of a BETi applied topically is above about 0.01% by weight of the composition. In some embodiments, the amount of a BETi applied topically is about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, or about 0.1% by weight of the composition. In some embodiments, the amount of a BETi applied topically is about 0.0015%, about 0.0025%, about 0.0035%, about 0.0045%, about 0.0055%, about 0.0065%, about 0.0075%, about 0.0085%, about 0.0095%, about 0.015%, about 0.025%, about 0.035%, about 0.045%, about 0.055%, about 0.065%, about 0.075%, about 0.085%, about 0.095%, about 0.1%, about 0.11%, 0.12%, 0.13%, 0.14%, 0.15%, 0.16%, 0.17%, 0.18%, 0.19% and 0.2% by weight of the composition. In some embodiments the amount of a BETi administered locally, e.g., into a joint is about 0.01% to about 15% by weight of the composition. In some embodiments the amount of a BETi administered locally is about 0.02% to about 10% by weight of the composition. In some embodiments the amount of a BETi administered locally is about 0.05% to about 5% by weight of the composition. In some embodiments the amount of a BETi administered locally is about 0.1% to about 10% by weight of the composition. In some embodiments the amount of a BETi administered locally is about 0.1% to about 5% by weight of the composition. In some embodiments the amount of a BETi administered locally is about 0.2% to about 2.5% by weight of the composition. In some embodiments the amount of a BETi administered locally is about 0.5% to about 1% by weight of the composition. In some embodiments the amount of a BETi administered locally is about 0.1% to about 5% by weight of the composition. In some embodiments the amount of a BETi administered locally is about 0.1% to about 10% by weight of the composition. In some embodiments the amount of a BETi applied administered locally is about 0.1% to about 5% by weight of the composition. In some embodiments the amount of a BETi administered locally is above about 0.01% by weight of the composition. In some embodiments the amount of a BETi administered locally is above about 0.05% by weight of the composition. In some embodiments the amount of a BETi administered locally is above about 0.01% by weight of the composition. In some embodiments the amount of a BETi administered locally is about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, or about 0.1% by weight of the composition. In some embodiments, the amount of a BETi administered locally is about 0.0015%, about 0.0025%, about 0.0035%, about 0.0045%, about 0.0055%, about 0.0065%, about 0.0075%, about 0.0085%, about 0.0095%, about 0.015%, about 0.025%, about 0.035%, about 0.045%, about 0.055%, about 0.065%, about 0.075%, about 0.085%, about 0.095%, about 0.1%, about 0.11%, 0.12%, 0.13%, 0.14%, 0.15%, 0.16%, 0.17%, 0.18%, 0.19% and 0.2% by weight of the composition.

In some embodiments, the amount of a BETi applied topically is about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, or about 0.1%. In some embodiments, the amount of a BETi applied topically is about 0.1%, about 0.11%, about 0.12%, about 0.13%, about 0.14%, about 0.15%, about 0.16%, about 0.17%, about 0.18%, about 0.19% or about 0.2% by weight of the composition. In some embodiments, the amount of a BETi applied topically is about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1.1%, about 1.2%, about 1.3%, about 1.4%, about 1.5% about 1.6%, about 1.7%, about 1.8%, about 1.9%, about 2%, about 2.25%, about 2.5% or about 3% by weight of the composition. In some embodiments, the amount of a BETi applied topically is about 0.0015%, to about 0.02% or about 0.004% to about 0.01% by weight of the composition. In some embodiments, the amount of a BETi applied topically is between about 0.005%, and about 0.02% by weight of the composition. In some embodiments, the amount of a BETi administered locally is about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, or about 0.1%. In some embodiments, the amount of a BETi administered locally is about 0.1%, about 0.11%, about 0.12%, about 0.13%, about 0.14%, about 0.15%, about 0.16%, about 0.17%, about 0.18%, about 0.19% or about 0.2% by weight of the composition. In some embodiments, the amount of a BETi administered locally is about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1.1%, about 1.2%, about 1.3%, about 1.4%, about 1.5% about 1.6%, about 1.7%, about 1.8%, about 1.9%, about 2%, about 2.25%, about 2.5%, or about 3%, or about 4%, or about 5%, or about 6%, or about 7%, or about 8%, or about 9%, or about 10% by weight of the composition. In some embodiments, the amount of a BETi administered locally is about 0.0015%, to about 0.02% or about 0.004% to about 0.01% by weight of the composition. In some embodiments, the amount of a BETi administered locally is between about 0.1%, and about 10% or about 0.4%, and about 4% by weight of the composition.

In one or more embodiments, for example, the amount of BETi is from about 0.01% to about 1.1% increasing in about 0.1% or about 0.2% increments. In some embodiments, for example, the amount of BETi is from about 0.1% to about 15% or in such other amounts as described herein and increasing in about 0.1% or about 0.2% increments.

In some embodiments, for example, the amount of BETi is from about 0.001% to about 1% increasing in about 0.01% or about 0.02% or about 0.025 increments In some embodiments, for example, the amount of BETi is from about 0.005% to about 1.1% increasing in about 0.05% or about 0.075% or about 0.1% or about 0.2% or about 0.3% increments. In some embodiments, for example, the amount of BETi is from about 0.01 mg/mL to about 10 mg/mL increasing in about 0.001 mg/mL or about 0.002 mg/mL increments.

In some embodiments, a dose-dependent reduction in MMP-9 and soluble E-cadherin secretion is observed across the BETi treatment concentration range. In some embodiments, treatment with BETi 0.1% and 1% results in a statistically significant reductions in MMP-9 secretion when compared to stimulated/untreated control, e.g., at least about a 95% (e.g., about 94.7%) reduction in secreted MMP-9 for the BETi 1% treatment (p<0.0001). In some embodiments, the BETi is BETi1.

In some embodiments, the BETi (e.g., BETi1) is numerically superior to OPZELURA (ruxolitinib 1.5%) cream in reducing MMP-9 secretion.

In some embodiments, treatment with BETi1 0.1% and 1% reduces the release of soluble E-cadherin relative to stimulated/untreated control (1%, p<0.002). In some embodiments, the effect of OPZELURA (ruxolitinib 1.5%) cream on soluble E-cadherin is not statistically different from stimulated/untreated control.

In some embodiments, treatment with BETi1 at all concentrations results in a reduction in melanocytorhagy, e.g.:
Stimulated/untreated control demonstrates a significant loss in melanin content, relative to unstimulated control.
BETi1 substantially prevents the loss of melanin pigment in the basal layers of skin in a dose dependent manner.
Residual melanin levels for BETi1 1% are about 367% of stimulated/untreated control, retaining approximately 75% of melanin relative to unstimulated control.

In some embodiments, treatment with BETi1 downregulates several inflammation genes associated with the pathogenesis of vitiligo. In some embodiments, BETi1 reduces the expression of IL1-α and IL1-β inflammatory cytokine transcripts relative to stimulated/untreated control. In some embodiments, the WNT signaling pathway is downregulated for the stimulated/untreated control. In some embodiments, this pathway is upregulated for BETi1 at the 0.1% and 1% concentrations relative to stimulated/untreated control. In some embodiments, WNT signaling is impaired in lesional and non-lesional skin of patients with vitiligo due to oxidative stress and autoimmunity. Without being bound by theory, non-impaired WNT signaling has been shown to promote improved melanocyte regeneration (see Regazzetti et al, J. Inv. Derm (2015); 135:3105-3114).

In some embodiments, stimulated and vehicle (placebo)-treated RHE demonstrates an upregulation of MMP9, relative to unstimulated control.

In some embodiments, BETi1 reduces the expression of MMP9 in a dose-dependent manner with a maximal effect at the 1% concentration.

In some embodiments, BETi1 1% reduces the secretion of MMP9 by keratinocytes >about 95%, relative to stimulated vehicle control and numerically superior to Ruxolitinib 1.5%.

In some embodiments, stimulated and vehicle (placebo)-treated RHE demonstrate an upregulation of soluble E-cadherin, relative to unstimulated control. In some embodiments, BETi1 affects a dose-dependent reduction in solubilized E-cadherin. In some embodiments, BETi1 0.1% and 1% are superior to topical Ruxolitinib 1.5% (OPZELURA).

In some embodiments, BET inhibition can retard the loss of a melanocyte adhesion molecule and preserve melanocytes localization in the basal layer of skin. In some embodiments, stimulation with a cytokine cocktail (e.g., TNF-α and IFN-γ) reduces the expression of TRP-1 and thus the abundance of melanocytes in the basal layers of the RHE. In some embodiments, this confirms melanocytorhagy. In some embodiments, the BETi1 formulation at 1% is able to reverse/prevent the melanocytorhagy, e.g., by increasing TRP-1 in the basal layers following the treatment.

In some embodiments, stimulated and vehicle (placebo)-treated RHE exhibits a loss in melanin content, relative to unstimulated control. In some embodiments, BETi1 prevents the loss of melanin pigment in the basal layers of skin in a dose dependent manner. In some embodiments, the residual melanin levels for BETi1 1% is about 367% of stimulated control, retaining approximately 75% of melanin relative to unstimulated control. In some embodiments, BETi1 is capable of inhibiting the loss of E-cadherin and retaining melanocytes in the basal layer.

In one or more embodiments BETi1 shows a significant improvement in reducing both melanocyte loss and key inflammatory biomarkers involved in the pathogenesis of vitiligo. In one or more embodiments BETi1 shows upregulaton of the WNT signaling pathway. In one or more embodiments BETi1 is numerically superior to ruxolitinib 1.5% cream in reducing key inflammatory biomarkers involved in the pathogenesis of vitiligo. In one or more embodiments BETi1 shows a dose-dependent reduction in MMP-9 and soluble E-cadherin across the tested concentrations. In one or more embodiments BETi1 at each of the 0.1% and 1% concentrations shows statistically significant reductions in MMP-9 when compared to vehicle, with about a 95% reduction in secreted MMP-9 for the 1% treatment (p<0.0001). In one or more embodiments BETi1 at each of the 0.1% and 1% concentrations shows statistically significant reductions in the release of soluble E-cadherin relative to vehicle. In one or more embodiments BETi1 1% also shows a reduction in the secretion of MMP-9 and soluble E-cadherin relative to topical ruxolitinib cream, 1.5%. In one or more embodiments BETi1 at each of the 0.1% and 1% concentrations shows a substantial reduction in the loss of melanin pigment in the basal layers of skin. In one or more embodiments BETi1 1% shows a much higher retention of melanin pigment relative to stimulated control and approximately 75% of melanin pigment relative to unstimulated control. In one or more embodiments BETi1 0.1% and 1% shows a statistically significant reduction in the expression of inflammatory cytokines IL1-α and IL1-β relative to vehicle (1%, p<0.0005). These cytokines are well recognized as important contributors to the pathogenesis of vitiligo. In one or more embodiments BETi1 shows a significantly upregulated the WNT signaling pathway at the 0.1% and 1% concentrations relative to vehicle, with a 10-fold increase observed at the 1% concentration (p<0.01).

In one or more embodiments BETi1 shows a significant impact on reducing inflammatory biomarkers and melanocyte loss in an ex vivo human skin model of vitiligo. In one or more embodiments BETi1 treatment results in clear improvement in preventing melanocyte loss. In one or more embodiments BETi1 shows upregulation of the WNT signaling pathway, recognized as an important indicator of melanocyte regeneration. In one or more embodiments BETi1 is numerically superior to OPZELURA (ruxolitinib 1.5%) cream in reducing key inflammatory biomarkers involved in the pathogenesis of vitiligo. In one or more embodiments BETi1 substantially prevents the loss of melanin pigment in the basal layers of skin in a dose dependent manner. In one or more embodiments BETi1 1% retains approximately 75% of melanin pigment relative to unstimulated control. In one or more embodiments BETi1 significantly reduces the expression of inflammatory cytokines IL1-α and IL1-3 relative to stimulated control (p<0.0005). These cytokines are well recognized as important contributors to the pathogenesis of vitiligo. In one or more embodiments BETi1 significantly upregulates the WNT signaling pathway at the 0.1% and 1% concentrations relative to stimulated control (10-fold increase, p<0.01). The WNT family of proteins and its signaling pathway is recognized as an important indicator of melanocyte regeneration. In one or more embodiments the PAN BET inhibitors described herein can provide one, two or more or all of the aforesaid effects.

In some embodiments, the BETi are superior to intra-articular steroids as evidenced by comparison of treated and untreated paw thickness and arthritis scoring. In some embodiments, the BETi are superior to dexamethasone. In some embodiments, the BETi is BETi1.

In some embodiments, a marked inhibition of paw thickening for the BETi dose levels is observed. In some embodiments, treatment effect for BETi in treated animals is dose-dependent over the dose range.

Without being bound by any theory, administration of BETi ameliorates joint inflammation in a dose-dependent manner (i.e., the higher the dose the higher the delta paw thickness <BETi dose vs vehicle>).

In some embodiments, BETi1 reduces inflammatory biomarkers (such as cytokines) in a joint or joint related disorder or disease.

In some embodiments, treatment response is evaluated based on an assessment of joint (e.g., foot or paw) thickening or swelling (in millimeters) and arthritis scoring based on a five-point composite severity scale of redness, swelling of the ankles and wrists, and paw thickness. In some embodiments, arthritis scoring ranges from 0 (normal) to 4 (extensive signs and symptoms of arthritis). In some embodiments, BETi1 demonstrates statistically significant improvement in treatment response for both endpoints at 1 mg/kg and 10 mg/kg doses. In some embodiments, BETi1 demonstrates numerically superior results at the 1 and 10 mg/kg doses compared to leading injectable steroid, dexamethasone, in inhibiting inflammation in arthritic joints of mice. In some embodiments, BETi1 demonstrates marked inhibition of paw thickening at the 1 and 10 mg/kg doses. In some embodiments, the 1 and 10 mg/kg doses, the inhibition of paw thickening is statistically significant in the treated paw relative to the untreated rear paw on day 12 (p<0.01). In some embodiments, the 1 and 10 mg/kg doses, mean joint (e.g., foot or paw) thickness at baseline (day 0) was about 2.08 mm, for the 10 mg/kg dose, the average paw thickness at day 12 was about 3.48 mm in the untreated paw versus about 2.17 mm in the treated paw, representing about a 37.6% reduction and for the 1 mg/kg dose, the average paw thickness at day 12 was about 2.98 mm in the untreated paw versus about 2.18 mm in the treated paw, representing a 26.8% reduction.

In some embodiments, the results with these two doses are numerically superior to intra-articular dexamethasone and consistent with the systemic dexamethasone treatment.

In some embodiments, an arthritis score is provided. In some embodiments, limbs treatment with BETi1 at the 1 and 10 mg/kg dose levels have an average arthritis score of 0.57 and 0.67, respectively, or near-normal. In some embodiments, the arthritis score is significantly lower in the treated paw at both doses relative to the non-treated paws on day 12 (p<0.05). In some embodiments, at the 10 mg/kg dose, the average arthritis score was about 0.67 in the treated paw versus about 3.33 in the untreated paw, representing about a 79.9% reduction. In some embodiments, at the 1 mg/kg dose, the average arthritis score was about 0.57 in the treated paw versus about 2.43 in the untreated paw, representing about a 76.5% reduction. In some embodiments, the limbs are treated with intra-articular dexamethasone and receive an average arthritis score of 1.3, which indicates mild symptoms. In some embodiments, the subjects (e.g., animals) have signs of redness and swelling of the ankle/wrist or apparent redness and swelling limited to individual digits.

In some embodiments, improvement in signs and symptoms of joint inflammation in the limbs of the subjects (e.g., animals) treated with BETi1, and no treatment effect is observed in untreated limbs, suggesting a locally acting anti-inflammatory effect of BETi1. In some embodiments, subjects (e.g., animals) treated with systemic dexamethasone experience a treatment effect in all limbs. In some embodiments, subjects (e.g., animals) treated with systemic BETi can experience a treatment effect in all limbs. In some embodiments, the preclinical tolerability is determined based on changes in body weight. In some embodiments, subjects (e.g., animals) treated with systemic dexamethasone experience continued weight loss throughout the study while all other treatment groups experience improved body weights following recovery from the RA stimulant. In some embodiments, there is no further weight loss. In some embodiments, all subjects (e.g., animals) experience weight loss from day 4 to 6, coinciding with stimulating an acute inflammatory response to the lipopolysaccharide injection. In some embodiments, from day 6 through the end of the study all animal body weights improve, other than for those animals treated with systemic dexamethasone, indicative of a lack of tolerance to the systemically delivered glucocorticosteroid. In some embodiments, there is no treatment effect in untreated joint (e.g., foot or paw) in subjects (e.g., animals) that receive BETi1. In some embodiments, in the BETi1 treatment groups there is no improvement in signs and symptoms of joint inflammation in the untreated limbs of the subjects (e.g., animals), suggesting a locally acting anti-inflammatory effect of BETi1.

In some embodiments, there is provided a method for the treatment, amelioration or prophylaxis of an inflammatory and/or an autoimmune disease or disorder or a related disease or disorder thereto, the method comprising administering to a subject in need thereof a therapeutically effective amount of a compound of Formula (I) or a tautomer, stereoisomer or a mixture of stereoisomers, or a pharmaceutically acceptable salt, or hydrate, or deuterated derivative thereof:

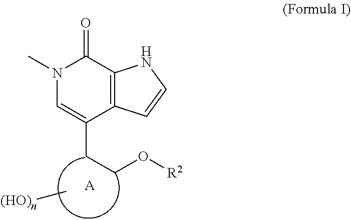

wherein:
ring A is selected from phenyl, N-methyl-2-pyridone, and thiazole;
n is 0 or 1, wherein when A is phenyl, n is 1; when A is N-methyl-2-pyridone, n is 0;
and when A is thiazole, n is 0;
R² is phenyl optionally substituted with 1 to 3 substituents independently selected from hydroxy, halo, C₁-C₆ alkyl, C₃-C₆ cycloalkyl, C₁-C₅ alkyloxy, C₁-C₅ alkylamino, C₁-C₆ fluoroalkyl, C₁-C₅ fluoroalkyloxy, and C₁-C₅ fluoroalkylamino; C₂-C₆ alkyl; and C₃-C₆ cycloalkyl optionally substituted with a substituent selected from C₁-C₆ alkoxy.

The method of any of the preceding embodiments, wherein the disease or disorder is selected from:
a. wounds, wounds resistant to healing, wounds swelling, burns, Pyoderma gangrenosum (PG), Generalized Pustular Psoriasis (GPP), and Palmar Plantar Pustulosis (PPP);
b. a pigmentation or pigmentation related disease or disorder;
c. a joint or joint related disease or disorder;
d. a respiratory or respiratory related disease or disorder; and
e. a fibrosis or fibrosis-associated disease or disorder.

The method of any of the preceding embodiments, wherein the pigmentation or pigmentation related disease or disorder is chosen from vitiligo, chemical leukoderma, tinea versicolor spots, albinism, and pityriasis alba, atrophie blanche, Griscelli syndrome, Halo moles, Hermansky-Pudlak syndrome, Hypomelanosis of Ito, Idiopathic guttate hypomelanosis, Leprosy, Leukoderma, Lichen sclerosus, Lupus erythematosus, Morphoea, Mycosis fungoides, Naevus anaemicus, Naevus depigmentosus, Piebaldism, Pityriasis versicolor, Poliosis, Postinflammatory hypopigmentation, Progressive macular hypopigmentation, Tuberous sclerosis (ashleaf spots), and Waardenburg syndrome.

The method of any of the preceding embodiments, wherein the pigmentation or pigmentation related disease or disorder comprises vitiligo.

The method of any of the preceding embodiments, wherein the joint related disease or disorder is chosen from arthritis, bursitis, Ehlers-Danlos syndrome, epicondylitis, Felty Syndrome, gouty arthritis, psoriatic arthritis, osteoarthritis, rheumatoid arthritis, Still's disease, tenosynovitis, synovitis, Sjögren's Syndrome, lyme disease, Whipple disease, bone cancer, lupus, and other autoimmune joint disorders.

The method of any of the preceding embodiments, wherein the joint or joint related disease or disorder comprises an arthritis.

The method of any of the preceding embodiments, wherein the arthritis comprises rheumatoid arthritis.

The method of any of the preceding embodiments, wherein the fibrosis or fibrosis-associated disorder and respiratory or respiratory related disorder comprises a lung disorder chosen from a pulmonary fibrosis (PF), idiopathic pulmonary fibrosis (IPF), desquamative interstitial pneumonia (DIP), acute interstitial pneumonia (ATP), nonspecific interstitial pneumonia (NSIP), respiratory bronchiolitis-associated interstitial lung disease (RB-ILD), cryptogenic organizing pneumonia (COP), and lymphoid interstitial pneumonia (LIP) or is a secondary lung disorder.

The method of any of the preceding embodiments, wherein the fibrosis or fibrosis-associated disorder and respiratory or respiratory related disorder comprises a pulmonary fibrosis (PF) or idiopathic pulmonary fibrosis (IPF).

The method of any of the preceding embodiments, wherein the PF is chosen from one or more of environmental PF, occupational PF, drug-induced PF, radiation-induced PF, autoimmune lung disease, and idiopathic PF.

The method of any of the preceding embodiments, wherein the fibrosis or fibrosis related disorder comprises scarring and scar formation.

The method of any of the preceding embodiments, wherein ring A is selected from

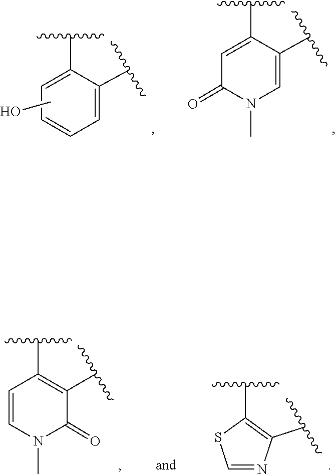

The method of any of the preceding embodiments, wherein ring A is selected from

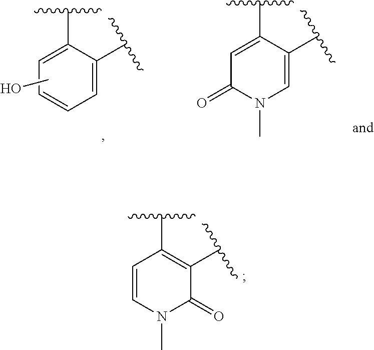

and
R² is phenyl optionally substituted with 1 to 3 substituents independently selected from hydroxy, halo, C₁-C₆ alkyl, C₃-C₆ cycloalkyl, C₁-C₅ alkyloxy, C₁-C₅ alkylamino, C₁-C₆ fluoroalkyl, C₁-C₅ fluoroalkyloxy, and C₁-C₅ fluoroalkylamino.

The method of any of the preceding embodiments, wherein the compound of Formula (I) is a compound of Formula (II) or a tautomer, stereoisomer or a mixture of stereoisomers, or a pharmaceutically acceptable salt, or hydrate, or deuterated derivative thereof:

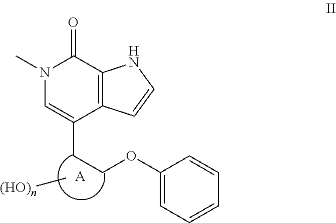

wherein:
ring A is selected from phenyl and N-methyl-2-pyridone; and
n is 0 or 1, wherein when A is phenyl, n is 1; and when A is N-methyl-2-pyridone, n is 0.

The method of any of the preceding embodiments, wherein ring A is selected from

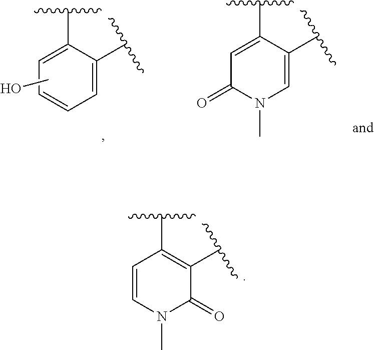

The method of any of the preceding embodiments, wherein the compound of Formula (I) is a compound of Formula (III) or a tautomer, stereoisomer or a mixture of stereoisomers, or a pharmaceutically acceptable salt, or hydrate, or deuterated derivative thereof:

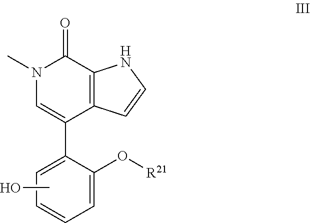

wherein:
R²¹ is selected from (i) phenyl optionally substituted with 1 to 3 substituents independently selected from hydroxy, halo, C₁-C₆ alkyl, C₃-C₆ cycloalkyl, C₁-C₅ alkyloxy, C₁-C₅ alkylamino, C₁-C₆ fluoroalkyl, C₁-C₅ fluoroalkyloxy, and C₁-C₅ fluoroalkylamino; and (ii) C₃-C₆ cycloalkyl optionally substituted with a substituent selected from C₁-C₆ alkoxy.

The method of any of the preceding embodiments, wherein R²¹ is (i) phenyl, (ii) cyclobutyl, or (iii) cyclohexyl optionally substituted with a substituent selected from C₁-C₆ alkoxy.

The method of any of the preceding embodiments, wherein R²¹ is phenyl.

The method of any of the preceding embodiments, wherein the compound is:

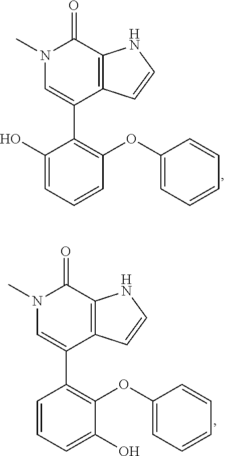

or a tautomer, stereoisomer or a mixture of stereoisomers, or a pharmaceutically acceptable salt, or hydrate, or deuterated derivative thereof.

The method of any of the preceding embodiments, wherein the compound is:

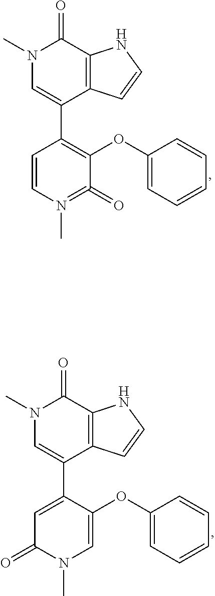

or a tautomer, stereoisomer or a mixture of stereoisomers, or a pharmaceutically acceptable salt, or hydrate, or deuterated derivative thereof.

The method of any of the preceding embodiments, wherein the compound is:

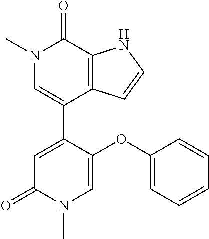 [SMILES: Cn1cc(Oc2ccccc2)c(-c2cn(C)c(=O)c3[nH]ccc23)cc1=O]

or a tautomer, stereoisomer or a mixture of stereoisomers, or a pharmaceutically acceptable salt, or hydrate, or deuterated derivative thereof.

The method of any of the preceding embodiments, wherein the compound is:

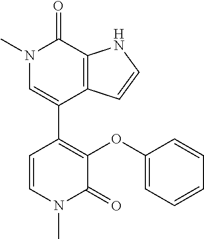 [SMILES: Cn1ccc(-c2cn(C)c(=O)c3[nH]ccc23)c(Oc2ccccc2)c1=O]

or a tautomer, stereoisomer or a mixture of stereoisomers, or a pharmaceutically acceptable salt, or hydrate, or deuterated derivative thereof.

The method of any of the preceding embodiments, wherein the compound is:

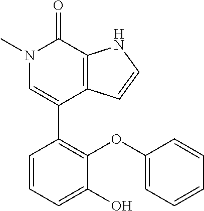 [SMILES: Cn1cc(-c2cccc(O)c2Oc2ccccc2)c2cc[nH]c2c1=O]

or a tautomer, stereoisomer or a mixture of stereoisomers, or a pharmaceutically acceptable salt, or hydrate, or deuterated derivative thereof.

The method of any of the preceding embodiments, wherein the compound is:

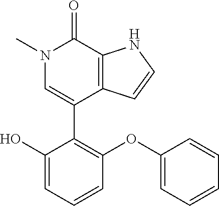 [SMILES: Cn1cc(-c2c(O)cccc2Oc2ccccc2)c2cc[nH]c2c1=O]

or a tautomer, stereoisomer or a mixture of stereoisomers, or a pharmaceutically acceptable salt, or hydrate, or deuterated derivative thereof.

The method of any of the preceding embodiments, wherein the compound is:

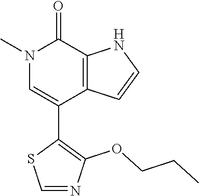 [SMILES: CCCOc1ncsc1-c1cn(C)c(=O)c2[nH]ccc12]

or a tautomer, stereoisomer or a mixture of stereoisomers, or a pharmaceutically acceptable salt, or hydrate, or deuterated derivative thereof.

The method of any of the preceding embodiments, wherein the compound is:

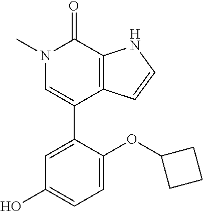 [SMILES: Cn1cc(-c2cc(O)ccc2OC2CCC2)c2cc[nH]c2c1=O]

or a tautomer, stereoisomer or a mixture of stereoisomers, or a pharmaceutically acceptable salt, or hydrate, or deuterated derivative thereof.

The method of any of the preceding embodiments, wherein the compound or a tautomer, stereoisomer or a mixture of stereoisomers, or a pharmaceutically acceptable salt, or hydrate, or deuterated derivative thereof is in the form of a pharmaceutical composition which comprises compound of Formula (I), or a pharmaceutically acceptable salt thereof, and a pharmaceutically acceptable carrier.

The method of any of the preceding embodiments, wherein the compound, tautomer, stereoisomer, pharmaceutically acceptable salt, hydrate, and/or deuterated derivative thereof has activity against one or more BET domains.

The method of any of the preceding embodiments, wherein there is an improvement in at least one symptom of the disorder or disease upon topical application of a therapeutically effective amount of compound, tautomer, stereoisomer, pharmaceutically acceptable salt, hydrate, and/or deuterated derivative thereof.

The method of any of the preceding embodiments, wherein upon administration of a therapeutically effective amount of the compound, tautomer, stereoisomer, pharmaceutically acceptable salt, hydrate, and/or deuterated derivative thereof there is a reduction in one or more cytokines that are elevated in inflammation.

The method of any of the preceding embodiments, wherein the one or more cytokines are chosen from SOX9, POMC, IL6, IL1a, IL1b, TNF, and EDN1 or chosen from IL-1β, IL17, IL6, IL36 and TNFα or chosen from IL-4, IL13, and IL-31 or chosen from IL-1β, IL17, TNF-α, IL8, IL-6 and IL-23.

The method of any of the preceding embodiments, wherein upon administration of a therapeutically effective amount of the compound, tautomer, stereoisomer, pharmaceutically acceptable salt, hydrate, and/or deuterated derivative thereof a therapeutic effect is associated with an upregulation the WNT pathway or signaling including WNT and/or upregulation of RABA3A.

The method of any of the preceding embodiments, wherein upon administration of a therapeutically effective amount of the compound, tautomer, stereoisomer, pharmaceutically acceptable salt, hydrate, and/or deuterated derivative thereof a therapeutic effect is associated with a reduction in MMP9 and/or in soluble E-cadherin.

The method of any of the preceding embodiments, wherein the disorder is a pigmentation disorder and upon administration of a therapeutically effective amount of the compound, tautomer, stereoisomer, pharmaceutically acceptable salt, hydrate, and/or deuterated derivative thereof a therapeutic effect is associated with a reduction in melanocytorhagy.

The method of any of the preceding embodiments, wherein a therapeutic effect is associated with a reduction in hydroxyproline levels and/or Ashcroft score.

The method of any of the preceding embodiments, wherein upon administration of a therapeutically effective amount of the compound, tautomer, stereoisomer, pharmaceutically acceptable salt, hydrate, and/or deuterated derivative thereof a therapeutic effect is associated with a reduction at the site of the disorder in infiltrates of lymphocytes, macrophages and neutrophils.

The method of any of the preceding embodiments, wherein the disorder is a fibrosis.

The method of any of the preceding embodiments, wherein there is a reduction in the number of fibroblasts and formation of collagen fibers.

The method of any of the preceding embodiments, wherein the disorder is a pulmonary fibrosis and the reduction is in the lung interstitium.

The method of any of the preceding embodiments, wherein the disorder is an arthritis and upon administration of a therapeutically effective amount of the compound, tautomer, stereoisomer, pharmaceutically acceptable salt, hydrate, and/or deuterated derivative thereof a therapeutic effect is associated with dose-dependent reduction in inflammation is a reduction in thickness or girth of a joint or limb.

The method of any of the preceding embodiments, wherein there is reduction in arthritic scoring or severity, and wherein the reduction in arthritic scoring or severity is a reduction in:
(a) definite redness and swelling of the ankle/wrist or apparent redness and swelling limited to individual digits, regardless of the number of affected digits;
(b) severe redness and swelling of the ankle/wrist;
(c) redness and swelling of the entire paw including digits; and/or
(d) maximally inflamed limb with involvement of multiple joints.

The method of any of the preceding embodiments, wherein the reduction is dose dependent.

The method of any of the preceding embodiments, wherein the reduction is by >about 50%.

The method of any of the preceding embodiments, wherein the upregulation is by >about 50%.

The method of any of the preceding embodiments, wherein the compound, tautomer, stereoisomer, pharmaceutically acceptable salt, hydrate, and/or deuterated derivative thereof is formulated as a suspension or partial suspension in the composition.

The method of any of the preceding embodiments, wherein the compound, tautomer, stereoisomer, pharmaceutically acceptable salt, hydrate, and/or deuterated derivative thereof is micronized.

The method of any of the preceding embodiments, wherein the compound, tautomer, stereoisomer, pharmaceutically acceptable salt, hydrate, and/or deuterated derivative thereof is in the form of nanoparticles.

The method of any of the preceding embodiments, wherein the compound, tautomer, stereoisomer, pharmaceutically acceptable salt, hydrate, and/or deuterated derivative thereof is solubilized or partially solubilized in the composition.

The method of any of the preceding embodiments, wherein the compound, tautomer, stereoisomer, pharmaceutically acceptable salt, hydrate, and/or deuterated derivative thereof or pharmaceutical composition is administered locally, topically or systemically.

The method of any of the preceding embodiments, wherein the compound, tautomer, stereoisomer, pharmaceutically acceptable salt, hydrate, and/or deuterated derivative thereof or pharmaceutical composition is administered by single injection, sequential injections, or infusion.

The method of any of the preceding embodiments, wherein the administration is into the epidural space, other spinal space, foramenal space, intraarticular space, lesions, periarticular space, perineum space, soft tissues, or at or near the location of inflammation, pain and/or damage.

The method of any of the preceding embodiments, wherein the compound, tautomer, stereoisomer, pharmaceutically acceptable salt, hydrate, and/or deuterated derivative thereof or pharmaceutical composition is administered by inhalation.

The method of any of the preceding embodiments, wherein the composition comprises, one or more ingredients chosen from PLGA microspheres, a carboxymethyl cellulose, a hyaluronic acid and a lubricant or a lubricin.

The method of any of the preceding embodiments, wherein when injected intraarticularly in a therapeutically effective amount the concentration of dissolved compound (e.g. there is no solid and/or suspension present and the compound is in solution) within the synovial fluid at 14 days is >about 3000 fold than that in the plasma and/or between days 7 to 21 ranges from ≥about 5000 fold to ≥about 1000 fold than that in the plasma.

Any discussion herein of documents, acts, materials, devices, articles, or the like is not to be taken as an admission that any or all of these matters form part of the prior art base or were common general knowledge in the field relevant to the present disclosure as it existed before the priority date of each claim of this application.

### EXAMPLES

### Section A:

### Experimental Method A: In-Vivo Psoriasis Animal Model

Thirty-six (36) female Balb/c mice are weighed and randomly divided into 9 groups (n=4) according to body weight on D1. For mice of native group 1 and tolerance groups 8 and 9, no induction of psoriasis is performed between D1 and D13. Psoriasis is induced in groups 2 to 7 by once daily topical application for 13 days (from day 1 to day 13) of 65 mg of Aldara™ cream (5.0% imiquimod) onto the shaved and epilated back of mice. Before the first treatment, a second randomization of mice of the 6 imiquimod-induced psoriasis groups is performed according to the severity of psoriasis (PASI), and also the body weight of mice. Starting on day 7 and until day 13, 100 mg of test formulations (groups 2 to 9) and 65 mg of vehicle (groups 1 and 2) are topically administered once daily for 7 consecutive days onto the shaved back skin of mice. Several measurements are taken during treatment period from day 7 to day 14: psoriasis index (PASI), body weight, use of enrichment and mortality. At the end of treatment period, TEWL is assessed, and blood and skin samples are collected. Histological analysis of skin samples is used for microscopic scoring of psoriasis and the determination of the thickness of parakeratosis and epidermis and blood analysis is used for assessing the level of proinflammatory biomarkers (cytokines assays). The effect of treatment of psoriasis induced mice with BETi formulations at the four dose levels are compared with Dermoval® cream containing 0.05% clobetasol propionate (reference) and vehicle (positive control) and also with healthy mice treated with vehicle (negative control).

PASI scoring takes into account the following 3 parameters: the presence of erythema on the skin, induration of the skin (thickening) and peeling of the skin at the area of the induction of psoriasis.

The presence of erythema on the skin at the area of induction of psoriasis with the following scoring grid:
0=no or no more erythema on the skin,
1=weak erythema on the skin,
2=moderate erythema on the skin,
3=important erythema on the skin.

The presence of induration of the skin at the area of induction of psoriasis with the following scoring grid:
0=no or no more induration of the skin,
1=weak induration of the skin,
2=moderate induration of the skin,
3=important induration of the skin.

The presence of peeling of the skin at the area of induction of psoriasis with the following scoring grid:
0=no or no more dryness/peeling of the skin,
1=weak dryness/peeling of the skin,
2=moderate dryness/peeling of the skin,
3=important dryness/peeling of the skin.

The sum of the mean scores of these 3 parameters result in the mean PASI score.

One of the issues with a steroid is that it causes skin thinning and continues to have an effect on no longer inflamed skin and the model set up and measurements above are not designed to reflect this negative effect. Therefore, a modified PASI (mPASI) is calculated in the same the manner as mean PASI score but excludes the scoring for induration (thickening of the skin).

Photographs of the back of each mouse are taken with a camera starting 7 days after the onset of psoriasis induction, after completing half of the treatment (D10), and then the day of sacrifice (D14).

Mice are weighed three times during the week of D1 to D7, and then daily during the week of D7 to D13, as well as on the day D14 when they are sacrificed. It should be noted that a characteristic body weight loss of mice is observed after the start of the induction of psoriasis, mainly during the first days following it. The body weight loss can reach between 10 to 15%.

The viability and the general behavior of mice of each cage of mice is recorded daily. Mice are autopsied and organs showing abnormalities are checked, collected, fixed and kept in a fixative solution.

The tolerance assessment to treatments with 0.01% and 1.0% BETi is performed daily during the treatment period (D7 to D13) as well as on the day mice are sacrifice (D14), and takes into account the following 5 parameters:

The presence of erythema on the skin with the following scoring grid:
0=no or no more erythema on the skin,
1=weak erythema on the skin,
2=moderate erythema on the skin,
3=important erythema on the skin,
4=severe erythema on the skin.

The presence of induration of the skin with the following scoring grid:
0=no or no more induration of the skin,
1=weak induration of the skin,
2=moderate induration of the skin,
3=important induration of the skin,
4=severe induration of the skin.

The presence of ulceration of the skin with the following scoring grid:
0=no or no more ulceration of the skin,
1=weak ulceration of the skin,
2=moderate ulceration of the skin,
3=important ulceration of the skin,
4=severe ulceration of the skin.

The presence of necrosis of the skin with the following scoring grid:
0=no or no more necrosis of the skin,
1=weak necrosis of the skin,
2=moderate necrosis of the skin,
3=important necrosis of the skin,
4=severe necrosis of the skin.

The presence of peeling of the skin with the following scoring grid:
0=no or no more peeling of the skin,
1=weak peeling of the skin,
2=moderate peeling of the skin,
3=important peeling of the skin,
4=severe peeling of the skin.

Any other abnormal observation is noted in the study notebook. Photographs of the back of mice are taken with a camera in case of abnormal observations.

For the duration of the experiment, dental rolls are used as enrichment and placed in each cage and replaced twice. Between D1 and D7, the number of dental rolls used by animals are determined twice in the morning, and are determined daily during D7 to D14, at the time of PASI or the tolerance assessment. The use of dental rolls is scored from 0=no use of dental rolls to 4=use of the 4 dental rolls. In the case where only half of a dental roll is used, a score of 0.5 is assigned. This allows to calculate a mean score of the use of dental rolls prior to treatment and during treatment period between (D7-D14).

Twenty-four (24) hours after the last treatment and before the sacrifice of mice, on D14, the measurement of the TEWL is performed on all mice, using the Tewameter® TM Hex (Courage+Khazaka electronic GmbH, Koln, Germany) and based on the “open chamber” measurement to evaluate the water barrier function of the skin linked to skin hydration. TEWL value, expressed in g/h/m2.

Mice are placed under gas anesthesia with isoflurane in a room maintained under the conditions similar to the animal cages. Three (3) consecutive measurements are performed on each mouse at the area of induction (or no induction) of psoriasis and treatments, on the upper, middle and lower back.

Immediately after the TEWL measurements on D14, mice are maintained under gas anesthesia and blood is taken from each mouse by intracardiac puncture. Blood samples are placed at +4° C. for 20 to 30 minutes before being centrifuged at 3000 rpm and +4° C. for 20 minutes. Serum is collected, frozen at −20° C. and then stored at −80° C. awaiting serum biomarkers assays (cytokines).

Immediately after the blood sampling and sacrifice of mice, the back skin of all mice is excised, and samples are fixed flat with buffered formalin solution in a Petri dish and stored in flasks containing the same buffered formalin solution before the performing of histological analysis.

After performing the skin sampling, a macroscopic observation of all organs of the abdominal and thoracic cavities is performed on all mice. Any other organ with abnormal shape, size or color is listed.

Assays of the 8 pro-inflammatory cytokines IL-1β, IL-6, IL-8, IL-17, IL-18, IL-23, IL-36 and TNF-α associated with psoriasis and other inflammatory diseases is performed on the isolated and frozen serum samples from blood collected at the end of the experiment on mice of the 7 experimental groups 1 to 7.

The Luminex® technique using magnetic beads technology and ProcartaPlex® immunoassay kit from the Invitrogen division of Thermofisher Scientific is used to perform the assays of the 7 multiplexed cytokines IL-1β, IL-6, IL-8, IL-17, IL-18, IL-23, and TNF-α. Analyte-specific antibodies for the 7 cytokines is pre-coated onto color-coded magnetic microparticles. Microparticles, standards and serum samples (non-diluted as recommended in the instruction manual) is pipetted into wells and the immobilized antibodies bind the analytes of interest. After washing away any unbound substances, a biotinylated antibody cocktail specific to the analytes of interest is added to each well. Following a wash to remove any unbound biotinylated antibody, streptavidin-phycoerythrin conjugate (Streptavidin-PE), which binds to the biotinylated antibody, is added to each well. A final wash removes unbound Streptavidin-PE and the microparticles is resuspended in buffer and read using a Bio-Rad Bio-Plex 200 Analyzer. A magnet in the analyzer captures and holds the superparamagnetic microparticles in a monolayer. Two spectrally distinct Light Emitting Diodes (LED) illuminate the microparticles. One LED identifies the analyte detected and the second LED determines the magnitude of the PE-derived signal, which is in direct proportion to the amount of the analyte bound. Each well is imaged with a Charged Coupled Device (CCD) camera. A representative standard curve from the 7 cytokines is diluted in a serial fourfold steps in specific standard buffer. Each standard, control and serum sample is analyzed in duplicate.

Quantitative sandwich Enzyme-Linked ImmunoSorbent Assay (ELISA) using mouse IL-36 immunoassay kit from the Invitrogen division of Thermofisher Scientific is used to perform the assay. A purified anti-mouse polyclonal antibody against the specific antigen IL-36 pre-coated onto a microplate is used. Standard, control, and serum samples, as recommended in the instruction manual, are pipetted into the wells and any mouse specific antigen present is bound by the immobilized antibody. After washing away any unbound substances, a biotin-conjugated anti-mouse IL-36 antibody is incubated in each well. Then, after washing, streptavidin-Horseradish Peroxidase (HRP) complex is added to the wells, allowing non-covalent biological interaction with biotin. Following several washes to remove any unbound complex, a TetraMethylBenzidine (TMB) substrate solution is added to the wells. The enzyme reaction yields a blue product that turns yellow when the stop solution is added. The intensity of the colour is measured at 450 nm and 570 nm using a microplate reader. The values of 570 nm are subtracted from those of 450 nm and data is in proportion to the amount of mouse IgE bound in the initial step. The serum samples values are then read off the standard curves. A representative standard curve from IL-36 is diluted in a serial two folds step in assay buffer. Each standard, control and serum sample is analyzed in duplicate.

Formalin-fixed skin samples are included and embedded in paraffin using a VIP apparatus. For each paraffin block, cross sections of 5 μm are performed using a microtome on 4 to 6 different zones per skin sample including the area of induction of psoriasis. Slides are placed in a rack, deparaffinized in xylene, rehydrated in 100% ethanol, 95% ethanol, 70% ethanol, 50% ethanol and then in running cold tap water. Slides are then stained with haematoxylin and eosin, dehydrated in 50% ethanol, 70% ethanol, 95% ethanol, 100% ethanol and xylene and mounted using an organic Euckitt mounting medium. The histopathological analysis of skin samples is performed blindly in order to determine microscopically the effects of compositions comprising BETi and clobetasol propionate (Dermoval®) cream on the animal model. The microscopic analysis of the different cutaneous planes, epidermis, dermis and hypodermis, are done on 10 cut planes of the 4 to 6 cross sections performed for each skin sample allowing to define a global microscopic score and also to estimate the thickness of parakeratosis and epidermis. The global microscopic score is the sum of several parameters observed and quantified in the epidermis, dermis and hypodermis, and defining the level of the disorder and the effect of treatment.

The body weight, mean body weight change (MBWC) of mice, the scores of the presence of erythema on the skin, induration of the skin and peeling of the skin at the area of induction of psoriasis, the score of the severity of psoriasis (PASI), the score of the use of enrichment, and the serum levels of the 8 pro-inflammatory cytokines IL-1β, IL-6, IL-8, IL-17, IL-18, IL-23, IL-36 and TNF-α, the microscopic scores of psoriasis, and the thickness of parakeratosis and epidermis are analyzed at the end of the experimentation. An ANalysis Of the VAriance (ANOVA) is performed in non-parametric mode using the Kruskal-Wallis test as a between-group variable comparison followed in case of significance by the Mann-Whitney U-test to compare the BETi treatment groups to negative and positive control groups and clobetasol propionate (Dermoval®) cream treated reference group. Results are expressed as mean±standard error of the mean (SEM). Differences are considered significant at the level of P<0.05. All statistical analyses are carried out using the StatView®5 statistical package (SAS, Institute, Inc., USA).

### Experimental Method B: In-Vivo Wound Healing Animal Model

Incisional wounds (10 mm) are made over the entire thickness of the skin (about 1 mm) on both flanks of nine female Hairless Skh-1 mice divided into 3 groups (n=3) according to body weight on D0. Formulations or hydroalcoholic gel are administered topically once daily starting immediately after the induction of skin wounds on DO until the complete healing of both wounds of each mouse, at the same time in the morning. The effects of treatment on wound healing with the formulation used containing one dose of the novel BETi, the vehicle of this new compound (negative control) and a hydroalcoholic gel preventing wound healing (positive control), is evaluated by the daily scoring of the healing process externally. The scoring is based on the daily measurement of the length and width, swelling of each induced skin wound from DO until complete healing, the determination of the time required for complete healing of each of induced skin wounds, and after complete healing of both wounds of a same mouse, by the scoring of the healing process internally by evaluation of the visibility of the scar, the presence of a tissue mass (collagen+elastin) under the scar and the level of vascularization of the scar.

Mice are weighed 3 times per week from the day of induction of skin wounds (D0) until the complete healing of both wounds of each mouse and before sacrifice. The viability and the well being of mice of each cage is checked and recorded daily.

The scoring takes into account the following 4 parameters: The length of the skin wounds measured with a ruler.

The width of the skin wounds with the following scoring grid:
0=skin wound completely closed,
1=skin wound almost completely closed,
2=skin wound moderately closed,
3=skin wound wide open.

The swelling of the skin wounds with the following scoring grid:
0=skin wound no or no longer swollen,
1=skin wound slightly swollen,
2=skin wound moderately swollen,
3=skin wound highly swollen.

The visibility of the skin wounds with the following scoring grid:
0=skin wound no or no longer visible,
1=skin wound slightly visible,
2=skin wound moderately visible,
3=skin wound highly visible.

The sum of the scores of these 4 parameters gives the Global External Score of Wound Healing (GESWH). The time required for complete healing of each induced skin wound is also determined.

After the complete healing of both skin wounds of each mouse, mice are sacrificed, and the scoring of the healing process internally is performed for each wound and takes into account the following 3 parameters:
visibility of the scar with the following scoring grid:
0=scar highly visible
1=scar moderately visible,
2=scar slightly visible
3=scar non-visible.

The presence of a tissue mass (collagen+elastin) under the scar with the following scoring grid:
0=no tissue mass present,
1=small tissue mass present,
2=moderate tissue mass present,
3=large tissue mass present.

The level of vascularization of the scar with the following scoring grid:
0=important vascularization of the scar,
1=moderate vascularization of the scar,
2=low vascularization of the scar,
3=very little vascularization of the scar.

The sum of the scores of these 3 parameters gives the Global Internal Score of Wound Healing (GISWH). A macroscopic observation of all organs of the abdominal and thoracic cavities is performed on all mice. Any other organ with abnormal shape, size or colour is listed.

The body weight, mean body weight change (MBWC) of mice, the external scores of the length, width, swelling and visibility of skin wounds as well as the Global External Score of Wound Healing (GESWH), the time required for complete healing of skin wounds, and the internal scores of the visibility, the presence of a tissue mass and the vascularization of scars as well as the Global Interna Score of Wound Healing (GISWH) are analyzed at the end of the experimentation. An ANalysis Of the VAriance (ANOVA) is performed in non-parametric mode using the Kruskal-Wallis test as a between-group variable comparison followed in case of significance by the Mann-Whitney U-test to compare the BETi formulation treated group to the vehicle and hydroalcoholic gel control groups. Results are expressed as mean±standard error of the mean (SEM). Differences are considered to be significant at the level of P<0.05. All statistical analyses are carried out using the StatView®5 statistical package (SAS, Institute, Inc., USA).

### Experimental Method C: Human Ex-Vivo Th2 and Th17 Stimulation of Human Skin Explants

Human skin biopsy tissue was stimulated to induce a Th17 immuno-phenotype using a method derived from Feghali-Bostwick et al. Induction of a Th17 phenotype in human skin—a mimic of dermal inflammatory diseases, Methods and Protocols, 2, 45 (2019). The pharmacodynamic assay described therein involves a similar paradigm to elicit a Th2 response and is used to evaluate the therapeutic potential of the active compounds (e.g., BETi1; and

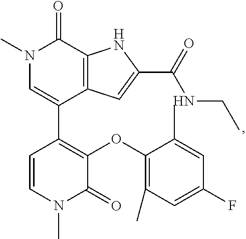 [SMILES: CCNC(=O)c1cc2c(-c3ccn(C)c(=O)c3Oc3c(C)cc(F)cc3C)cn(C)c(=O)c2[nH]1]

hereinafter “BETia”) in topical formulations for dermatological diseases.

Freshly excised healthy human skin tissue from abdominoplasties is defatted, cleaned and sectioned into 7 mm biopsies. The biopsies are placed in Transwell® inserts with the epidermis apical and exposed to air and the dermis submerged in media in the basal chamber. The biopsies are pre-treated overnight at 37° C., 5% CO2 with different compounds and controls added to the media in the basal chamber.

The next day, contents of the basal chamber is replaced with fresh media containing the test compound (listed below) and a stimulation cocktail for either Th2 inflammation (proprietary of service provider cocktail) or Th17 inflammation (mix of antibodies against CD3, CD28, IL-4, IFNγ and recombinant IL-1β, IL-6, IL-21, TGF-β). The biopsies are incubated at 37° C., 5% CO2 for a further 24 hrs.

A list of all the treatments is provided below:
1. Untreated control (for both stimulations)
2. Th2 stimulated control
3. Th17 stimulated control
4. Th2+BETia at 0.01, 0.04, 0.16, 0.63, 2.50, and 10 μM
5. Th2+BETi1 at 0.01, 0.04, 0.16, 0.63, 2.50, and 10 μM
6. Th2+Ruxolitinib at 0.01, 0.04, 0.16, 0.63, 2.50, and 10 μM
7. Th2+betamethasone valerate 210 μM (equivalent to 0.01%)
8. Th17+BETia at 10 μM
9. Th17+BETi1 at 10 μM
10. Th17+Ruxolitinib at 10 μM
11. Th17+betamethasone valerate 210 μM (equivalent to 0.01%)

After harvesting, the biopsies are cut in half, and one half is homogenized and used for RNA extraction by standard methods and inflammatory pathways is assessed by RT-qPCR. The ability of BETi1 or BETia to inhibit the activity of up to six biomarkers per stimulation is reported as percent activity calculated to stimulated control and fold change in gene expression calculated to untreated control with respect to Th2 (IL4, IL13, IL31, CCL17, CCL26, CCL2) and Th17 (CXCL10, IL17α, IL22, IL36γ, CCL20, SerpinB4). Statistical analysis is performed on data sets from all 3 donors combined and compares the percent inhibition of each biomarker by each treatment calculated to its respective vehicle.

qPCR values were calculated using the relative quantification approach outlined by Livak, et al (2001) (Section 9, Reference 2). The Ct values (threshold cycle) defined as the fractional cycle number at which the amount of amplified target reaches a fixed threshold, were normalised to the Ct value of an internal biomarker or ‘housekeeping gene’ (GAPDH). The higher limit of detection was set at a Ct value of 40, and any target having not reached Ct by 40 cycles was set to this value. The delta Ct values (dt(Ct)) refers to the Ct of the gene of interest minus the Ct of the internal biomarker. The ddt(Ct) is the dt(Ct) value of the gene minus the average dt(Ct) value of the untreated samples. The ddt(Ct) is converted to power by the equation; =power(2,dt(Ct)). Fold change was calculated as ratio of the power of the gene of interest divided by the average power of the untreated samples. This normalizes the untreated samplers to a value of 1. For the comparison of multiple skin donors, the stimulated samples were normalized to 100% maximum activity (such to normalize variability between different skin donors). Percent activity was calculated as the percent increase or decrease in stimulation compared to the stimulated samples set at 100% maximum activity. Percent change was calculated as: Inhibition=(100−(avg % activity/stimulated)*100). Statistical analysis was performed using One-way ANOVA with Dunnett's Multiple Comparisons (non parametric).

### Experimental Method D: Formulation Preparation

Petrolatum is heated to approximately 70° C. in an appropriate beaker while being mixed. Petrolatum is fully melted to achieve clear liquid. API needed for preparation of the batch is placed in the beaker with preheated petrolatum and sonicated at approximately 70° C. for 30 minutes. The dispersion is then cooled to ambient temperature while being under overhead mixing.

Formulation of a hydrophobic carrier with multiple excipients can be achieved in a similar manner, where the hydrophobic excipients are heated to approximately 70° C. in an appropriate beaker while being mixed to achieve clear liquid. API needed for preparation of the batch is placed in the beaker with preheated excipients and sonicated or alternatively homogenized at approximately 70° C. for 30 minutes. The dispersion is then cooled to ambient temperature while being under overhead mixing. If a solution of API is desired the same procedure can be applied including one or more solvents in which the API has solubility (see e.g., Tables 39A and 39B). To the extent the excipients selected are liquid at a lower temperature the procedure can be carried out at that lower temperature until the API is completely dissolved.

Formulation of a hydrophilic carrier with API e.g., as a semi-solid carrier formulation in polyethylene glycol can be prepared as follows. The API is dissolved in low molecular weight polyethylene glycol e.g., PEG400 by mixing with overhead stirrer at ambient temperature. High molecular weight polyethylene glycol e.g., PEG3350 is heated to 70° C. while being mixed to generate clear melt. The solution of API in PEG400 is heated to approximately 60° C. and added to molten PEG3350 while being mixed by overhead stirrer and the mixture is cooled to 25° C. while being mixed.

### Experimental Method E: Chemical Stability

For Polarized Light Microscopy evaluation, the samples of the formulations in petrolatum are heated to 55° C. to melt most of the petrolatum crystals and make visible crystals of API. The API crystals are measured and photomicrographs are taken.

The microscopy samples of API are prepared by dispersing some (unmeasured amount) of API powder in a drop of mineral oil. The dispersion is done by a spatula. The concentration of API in mineral oil is unknown.

The stability of prepared formulations was assessed by HPLC testing of samples of two formulations, prepared with API from two different sources. One was exposed to 25° C. for 2.5 months and the other was exposed to 40° C. for 3 months. The area % of the main peak in the samples after temperature exposure was compared to area % of the main peak in initial samples of the same batches and in the corresponding APIs. The samples exposed to stability challenge were also evaluated for presence of new peaks, not observed in API or in initial samples.

### Example 1. In-Vivo Efficacy and Tolerance from a Murine Imiquimod-induced Inflammatory Model

Investigation of the effect of the different topical formulations comprising four concentrations of BETi1 compound (Table 1) on efficacy and tolerance in a Psoriasis animal model was undertaken in accordance with the protocol set out in Experimental Method A. Dorsal depilated mice were topically dosed with imiquimod cream to induce a Th17 inflammation pathology over a 7-day induction phase. A further 7-day treatment phase evaluated four doses of BETI1 (0.001%, 0.01% 0.1% and 1% concentrations) compared to a class 1 super-potent glucocorticosteroid product positive control (Dermoval®-clobetasol propionate 0.05% cream) and induced mice treated with vehicle (positive control). Further, an imiquimod-naive, healthy mice treated with vehicle (negative control group) was also included.

The investigation addressed, amongst other things, the effect of the different BETi1 concentrations (0.001%, 0.01%, 0.1% and 1.0%) on various parameters. Tolerance was tested at two BETi1 concentrations (0.01% and 1%) in healthy mice.

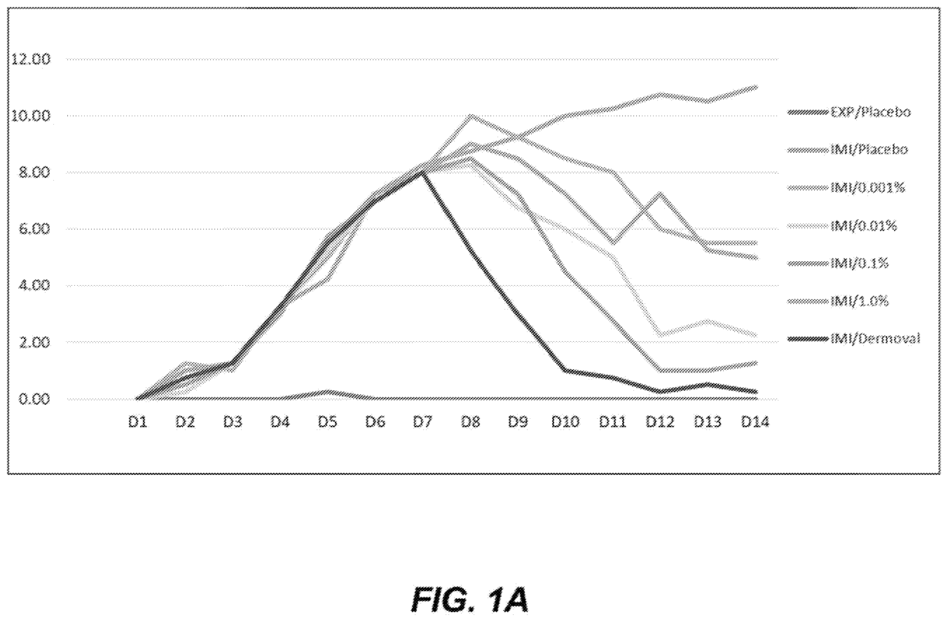

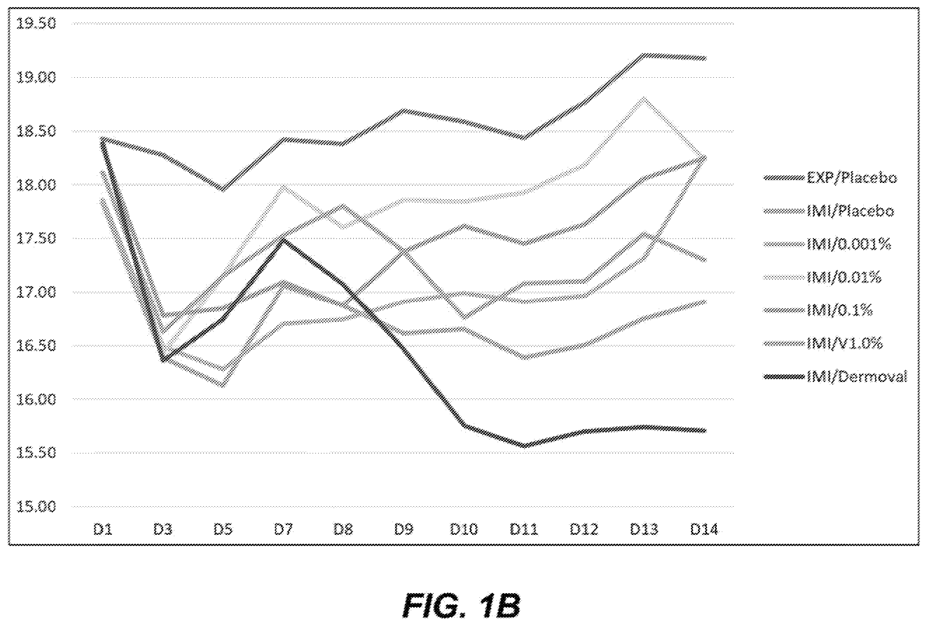

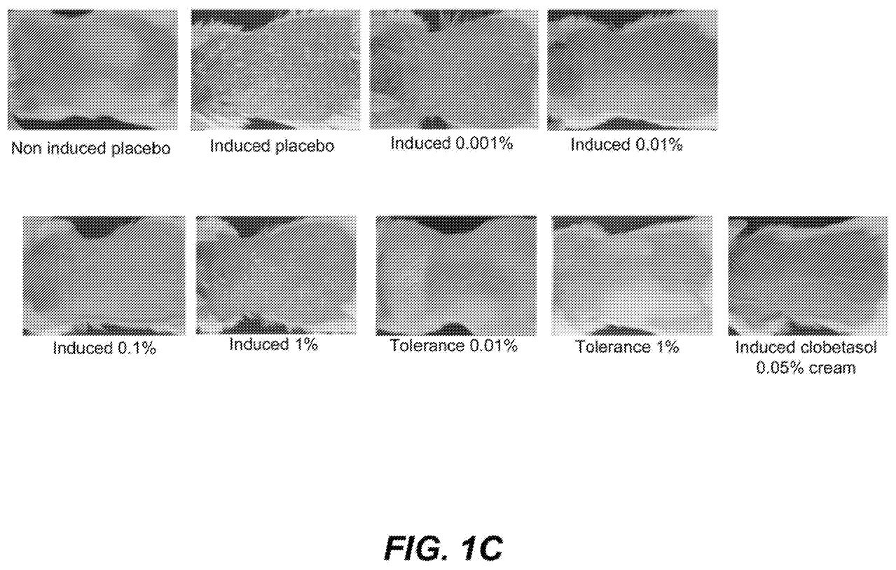

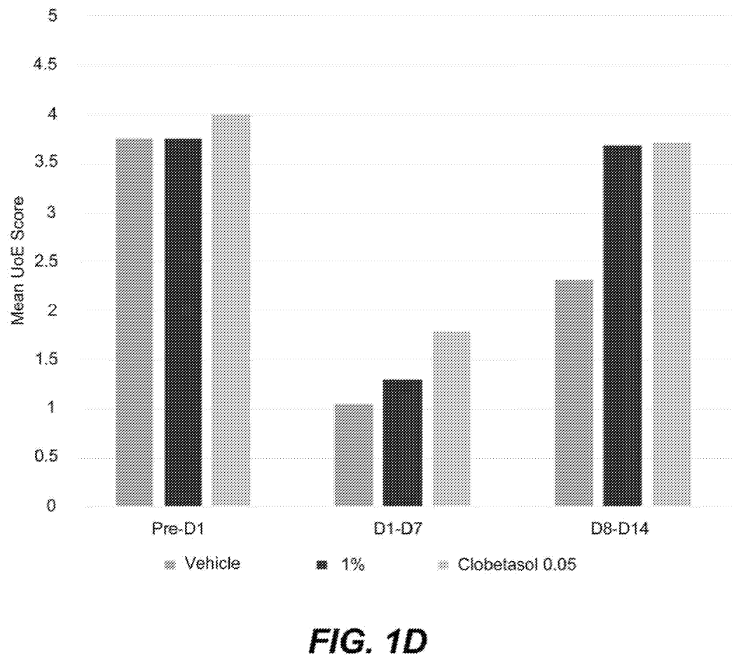

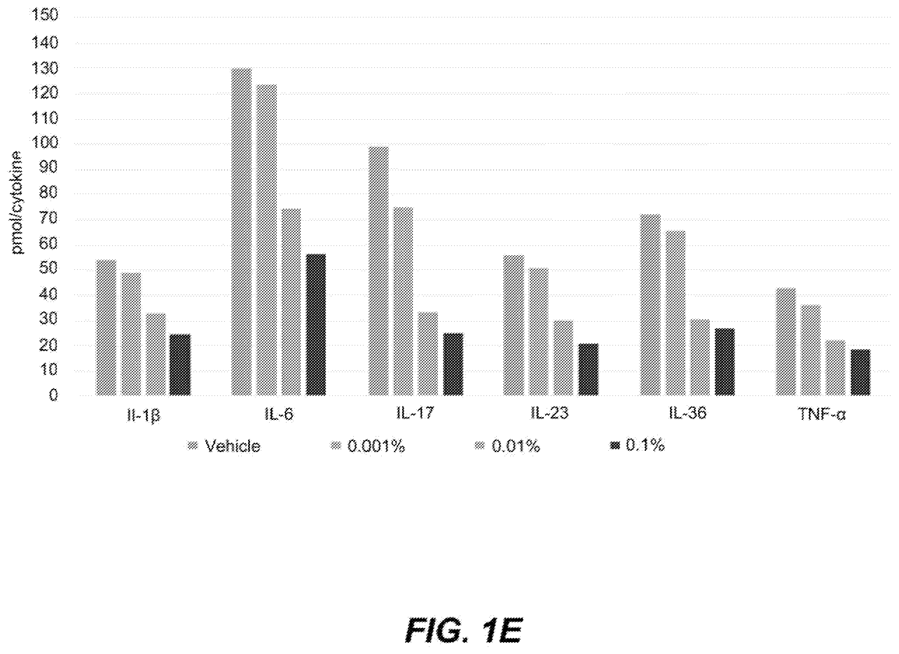

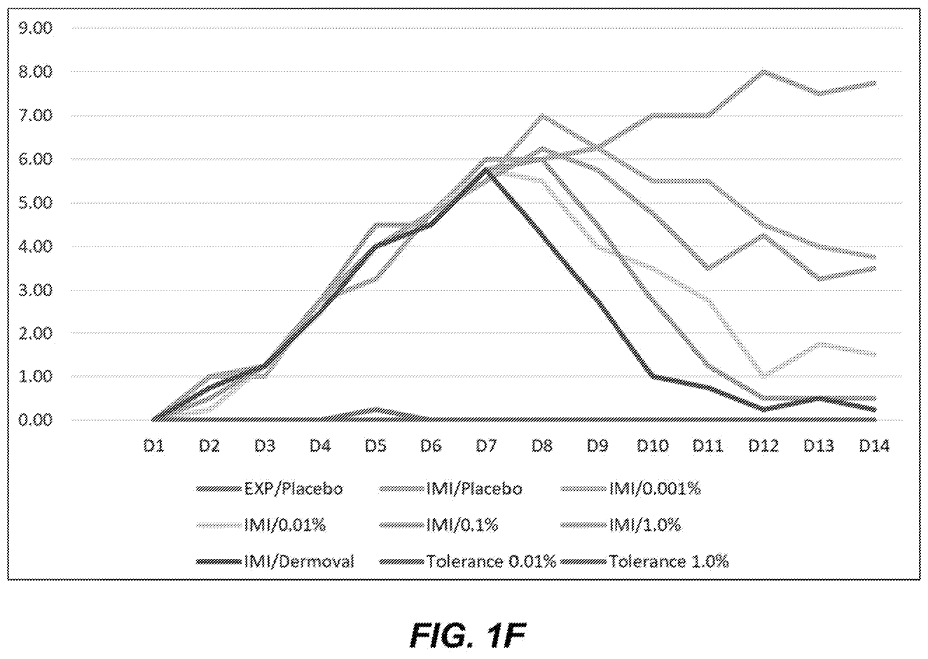

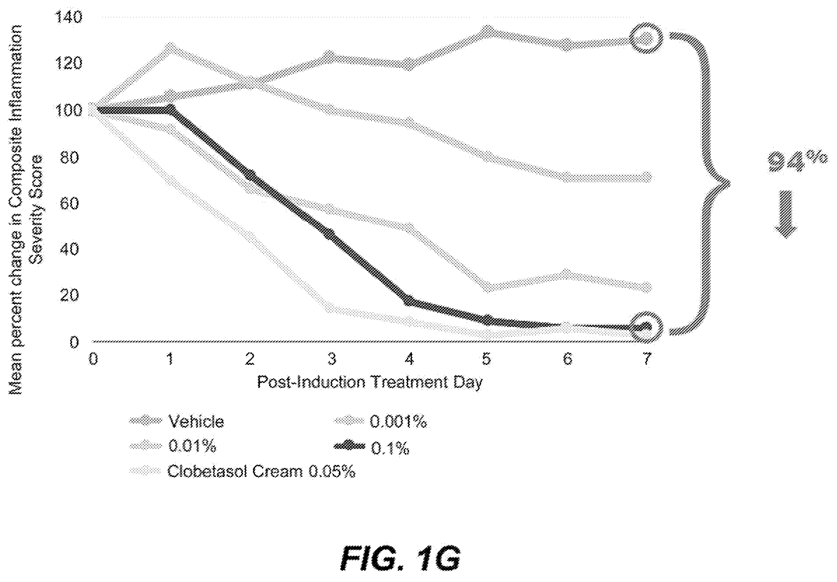

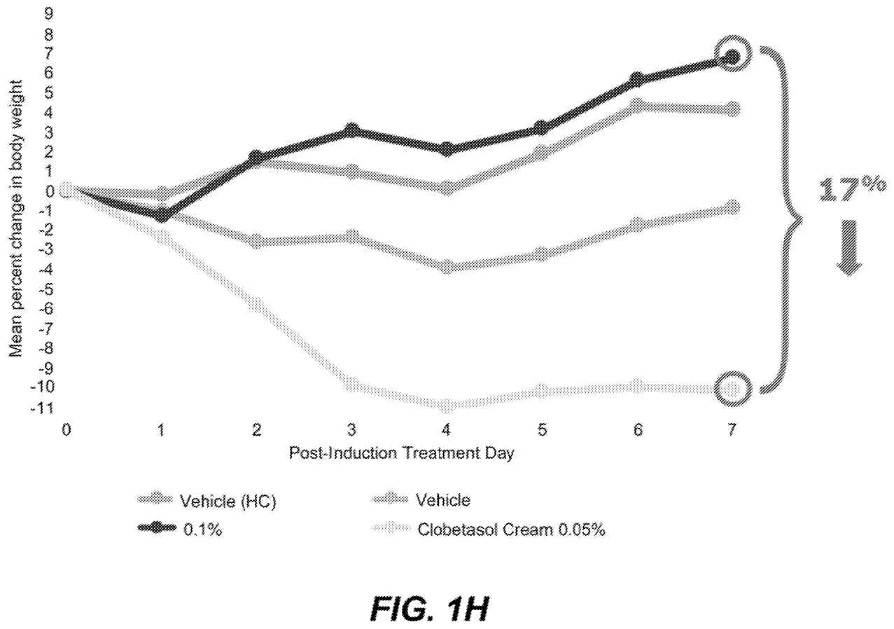

Measurements were taken during treatment period from day 7 to day 14. Mice treated with a BETi1 formulation presented beneficial effects in several of the parameters related to psoriasis. Such parameters include but are not limited to visual parameters and reduction in biomarkers associated with inflammation. A progressive reduction in the mean PASI score at BETi1 concentrations between 0.001% and 0.1% was observed from day 7 to day 14 (Table 1 and FIG. 1A) and as presented pictorially as of day 14 (FIG. 1C). As can be seen from FIGS. 1A and 1C and Table 1, formulations comprising BETi1 were effective in reducing the mean Psoriasis Index (PASI) score values and restoring healthy skin. BETi1 was shown to be highly selective allowing it to bind efficiently even at concentrations as low as 0.001%. A dose-response was observed for the BETi-formulations, within the range of 0.001% and 0.1%, with the highest efficacy (lowest PASI score) achieved at the 0.1% strength. The mean PASI score value for animals treated with vehicle was higher than the mean PASI score values for animals treated with BETi formulations or steroid cream. Although the reduction in mean PASI value for mice treated with 0.1% was slightly less than for mice treated commercial steroid cream clobetasol propionate (Dermoval®), treatment with Dermoval® reduced mean animal body weight compared to BETi treatments, (Table 2A, 2B and FIGS. 1B and 1C). In other words, animals treated with BETi1 0.1% continued to gain body weight in a similar manner to healthy control group treated with vehicle. Clobetasol cream 0.05% group had a 17% reduction in body weight compared to the BETi1 0.1% group at treatment day 7 (i.e. 107% for BETi1 compared to 90% for steroid at Day 14) (FIG. 1H and Table 2C). This indicates that the BETi1 treatments were better tolerated.

All animals treated during the induction phase with 7 days of topically-applied imiquimod experienced a mean reduction in use of cage enrichment compared to pre-induction phase. Vehicle only treatment group had a marginal increase in use of cage enrichment compared to the induction phase. Both BETi 1% and clobetasol 0.05% cream groups had recovered to pre-dose levels (Table 3 and FIG. 1D).

As seen in FIG. 1C no appreciable improvement in clinical signs was seen for animals treated with vehicle. In contrast, substantial resolution of clinical signs was observed in animals treated with BETi1 0.1%. Skin presented with normal physiology with no evidence of striae rubrae or atrophy and there was no evidence suggestive of intolerance. Substantial resolution of clinical signs was also observed in animals treated with a steroid. However, significant evidence of dermal atrophy (clear presence of both rhytides and deep wrinkles) and marked dermal translucency and elastolysis was also observed.

Without being bound by any theory it is postulated that results comparable to a steroid if not better may be obtained with BETi1 concentrations between from about under 0.1% to about 1% (e.g., about 0.07 to about 0.7%) while avoiding steroid untoward side effects such as weight loss and skin thinning.

Surprisingly, the PASI decreasing effect of BETi did not reach a plateau when the concentration of the BETi was increased to 1% but instead a decrease in the mean PASI value score was observed that was slightly better than the score achieved at the 0.001% strength. Without being bound by any theory it is postulated that at doses greater than 0.1% the selectivity decreases and BETi starts binding to other receptors in the skin activating pathways which prevent effective skin healing and which mask the effect shown at lower BETi concentrations.

As mentioned above the steroid was observed to cause thinning of the skin and this can be perceived to mask the scoring observed with the other parameters. Omitting the induration parameter (thickening of the skin) may on one level provide a clearer representation of the treatment potential of BETi1 since no marked weight loss or thinning was noted other than with the steroid. Thus, a modified PASI (mPASI) value was calculated.

The modified PASI (mPASI) or also referred as Composite Inflammation Severity Score is calculated in the same the manner as mean PASI score but excludes the scoring for induration. It is a composite mean score of erythema and peeling severity scored on a 4-point ordinal scale per domain (0=none, 1=mild, 2=moderate and 3=severe for a maximum score of 6).

The BETi1 0.1% had comparable efficacy with respect to reduction in mPASI score to a commercial cream containing 0.05% clobetasol as a reference (Dermoval®) with an earlier onset seen for the steroid (see FIG. 1F and Table 1B). There was a 94% reduction in mPASI score for BETi1 0.1% relative to vehicle control group at Day 7. The mean percentage change is expressed from initiation of treatment phase A dose dependent response was observed over the BETi1 concentration range 0.001% to 0.1%. BETi1 0.1% had comparable efficacy to a clobetasol propionate 0.05% cream (FIG. 1G, Table 1C).

Measurements to evaluate the water barrier function of the skin linked to skin hydration showed a correlation between the PASI score dose-response and the mean Trans-Epidermal Water Loss (TEWL) score for the BETi formulations (Table 4). It was shown that animal induced with the disease and treated with a vehicle exhibited a high TEWL, which is a sign of impaired skin barrier. Animal treated with a BETi formulation showed a reduced TEWL in line with the PASI score. Without being bound by any theory, it may be that treatment with a BETi formulation restored the skin barrier leading to reduced TEWL values.

Measurements to evaluate the level of pro-inflammatory biomarkers (IL-1β, TNF-α, IL-18, IL-6, IL-8, IL-17, IL-23, and IL-36) showed a strong correlation between improvement in clinical severity scores and reduction in many pro-inflammatory biomarkers relevant to Th17-mediated immune diseases. Dose-dependent reduction in biomarker expression was observed with BETi1 0.1% demonstrated as having the greatest effect. IL10 and IL-23 precipitate the polarization of naïve Th0 immune cells to Th17 cells. Th17 cells produce a range of cytokines that drive inflammation in immune diseases. These include IL17, IL6, IL36 and TNFα(Table 5 and FIG. 1E).

As can be seen from FIG. 1C and Table 1B healthy animals treated with either vehicle or BETi 0.01% or 1% did not experience any noticeable local skin reaction to treatment and there was no impact on fur re-growth. Thus, BETi1 is well tolerated even at the highest dose.

In summary, BETi1 exhibited anti-inflammatory effect in preclinical data similar to a super-potent glucocorticosteroid. BETi1 appeared well-tolerated in mice, as seen through animal body weight and skin condition. BETi1 also demonstrates stronger inhibition of key Th17 cytokines in ex vivo data with human skin tissue when directly compared to JAK1/2 inhibitor, ruxolitinib. A dose-dependent improvement in the signs and symptoms of inflammation was observed for BETi1 treatment groups. Treatment with BETi1 0.1% resulted in a 94% reduction in the composite inflammation severity score of erythema and scaling, relative to the vehicle control group at treatment day 7. Animals treated with BETi1 0.1% experienced a reduction in inflammation comparable to those treated with the class 1 super-potent glucocorticosteroid clobetasol propionate 0.05% cream. Corresponding dose-dependent reductions were observed across all treatment groups in Th17-relevant cytokine biomarkers of inflammation, including IL-1, IL-17, IL-6, IL-36 and TNFα for BETi1-treated animals. Treatment with BETi1 at all concentrations appeared well-tolerated based on the following observations: animals treated with BETi1 continued to gain body weight in a similar manner to the healthy control group, whereas animals treated with clobetasol propionate cream 0.05% had a mean body weight loss of approximately 17% compared to the animals treated with BETi1 0.1%; animals treated with BETi1 had no evidence of dermal tolerance issues, consistent with the healthy control group, whereas animals treated with clobetasol propionate 0.05% cream had significant skin atrophy as evidenced by deep wrinkling and rhytides (fine wrinkles), marked dermal translucency and loss of elasticity.

There are some elevated biomarkers involved in psoriasis which are also common in other inflammatory disorders such as Pyoderma gangrenosum (PG), Palmar Plantar Pustulosis (PPP) and Generalized Pustular Psoriasis (GPP). Neutrophils play a key role in PG and IL-1, IL17, TNF-α, IL8, IL-6 and IL-23 are known to be upregulated in PG lesions. Without being bound by any theory, a similar expression pattern of these biomarkers to those of psoriasis animal model would be consistent with the topical formulation comprising BETi being an effective treatment against PG, IL-1, IL-17 and IL-36 are the dominant cytokines increased in PPP. IL-1 and IL-36 are the dominant cytokines increased in GPP. Without being bound by any theory, the topical formulation comprising BETi may also be an effective treatment against PPP and GPP. Moreover, a topically applied BET inhibitor product may positively impact diseases involving multiple, diverse inflammatory cell signaling pathways that are active in rare neutrophilic dermatoses.

As discussed above treatment with BETi formulations presented beneficial effects in several of the parameters related to psoriasis and were well tolerated. Such parameters included but were not limited to visual parameters such as erythema, peeling and induration at the area of induction, behaviour parameters (general wellbeing and use of enrichment), reduction of water loss and reduction in the level biomarkers related to inflammation. Treatment with commercial steroid product did not show good tolerability, as evidenced herein by loss of body weight and skin thinning in psoriasis animal model treated with clobetasol propionate (Dermoval®). The BETi1 compound in a topical composition described herein avoided such undesirable side effects. Thus, the topical compositions herein can provide an effective, safe, well tolerated long term solutions to treat disorders like PG, GPP and PPP and possibly many other inflammatory diseases, unlike steroids which are preferably limited for use for short periods and are not well tolerated and can have undesirable side effects as indicated herein.

TABLE 1A
PASI Mean score values

| | | D 1 | D 2 | D 3 | D 4 | D 5 | D 6 | D 7 | D 8 | D 9 | D 10 | D 11 | D 12 | D 13 | D 14 |
|---|---|---|---|---|---|---|---|---|---|---|---|---|---|---|---|
| PASI | EXP/Vehicle | 0.00 | 0.00 | 0.00 | 0.00 | 0.25 | 0.00 | 0.00 | 0.00 | 0.00 | 0.00 | 0.00 | 0.00 | 0.00 | 0.00 |
| Mean | (negative | | | | | | | | | | | | | | |
| score | control) | | | | | | | | | | | | | | |
| values | IMI/Vehicle | 0.00 | 1.00 | 1.25 | 3.25 | 4.25 | 7.25 | 8.25 | 8.75 | 9.25 | 10.00 | 10.25 | 10.75 | 10.50 | 11.00 |
| | (positive | | | | | | | | | | | | | | |
| | control) | | | | | | | | | | | | | | |
| | IMI/BETi1 | 0.00 | 1.25 | 1.00 | 3.25 | 5.00 | 7.25 | 8.00 | 10.00 | 9.25 | 8.50 | 8.00 | 6.00 | 5.50 | 5.50 |
| | 0.001% | | | | | | | | | | | | | | |
| | IMI/BETi1 | 0.00 | 0.25 | 1.25 | 3.25 | 5.25 | 7.00 | 8.00 | 8.25 | 6.75 | 6.00 | 5.00 | 2.25 | 2.75 | 2.25 |
| | 0.01% | | | | | | | | | | | | | | |
| | IMI/BETI1 | 0.00 | 0.50 | 1.25 | 3.00 | 5.75 | 7.00 | 8.00 | 8.50 | 7.25 | 4.50 | 2.75 | 1.00 | 1.00 | 1.25 |
| | 0.1% | | | | | | | | | | | | | | |
| | IMI/BETI1 | 0.00 | 1.00 | 1.25 | 3.25 | 5.50 | 7.25 | 8.00 | 9.00 | 8.50 | 7.25 | 5.50 | 7.25 | 5.25 | 5.00 |
| | 1.0% | | | | | | | | | | | | | | |
| | IMI/Dermoval | 0.00 | 0.75 | 1.25 | 3.25 | 5.50 | 7.00 | 8.00 | 5.25 | 3.00 | 1.00 | 0.75 | 0.25 | 0.50 | 0.25 |
| | Tolerance | 0.00 | 0.00 | 0.00 | 0.00 | 0.00 | 0.00 | 0.00 | 0.00 | 0.00 | 0.00 | 0.00 | 0.00 | 0.00 | 0.00 |
| | BETi1 0.01% | | | | | | | | | | | | | | |
| | Tolerance | 0.0 | 0.00 | 0.00 | 0.00 | 0.00 | 0.00 | 0.00 | 0.00 | 0.00 | 0.00 | 0.00 | 0.00 | 0.00 | 0.00 |
| | BETi1 1.0% | | | | | | | | | | | | | | |

TABLE 1B
Modified PASI Mean score values

| | | D 1 | D 2 | D 3 | D 4 | D 5 | D 6 | D 7 | D 8 | D 9 | D 10 | D 11 | D 12 | D 13 | D 14 |
|---|---|---|---|---|---|---|---|---|---|---|---|---|---|---|---|
| Modified | EXP/Vehicle | 0.00 | 0.00 | 0.00 | 0.00 | 0.25 | 0.00 | 0.00 | 0.00 | 0.00 | 0.00 | 0.00 | 0.00 | 0.00 | 0.00 |
| PASI | IMI/Vehicle | 0.00 | 0.75 | 1.25 | 2.75 | 3.25 | 4.75 | 6.00 | 6.00 | 6.25 | 7.00 | 7.00 | 8.00 | 7.50 | 7.75 |
| Mean | IMI/BETi1 | 0.00 | 1.00 | 1.00 | 2.75 | 4.00 | 4.75 | 5.50 | 7.00 | 6.25 | 5.50 | 5.50 | 4.50 | 4.00 | 3.75 |
| score | 0.001% | | | | | | | | | | | | | | |
| values | IMI/BETI1 | 0.00 | 0.25 | 1.25 | 2.75 | 4.00 | 4.75 | 5.75 | 5.50 | 4.00 | 3.50 | 2.75 | 1.00 | 1.75 | 1.50 |
| | 0.01% | | | | | | | | | | | | | | |
| | IMI/BETI1 | 0.00 | 0.50 | 1.25 | 2.75 | 4.50 | 4.50 | 5.75 | 6.00 | 4.50 | 2.75 | 1.25 | 0.50 | 0.50 | 0.50 |
| | 0.1% | | | | | | | | | | | | | | |
| | IMI/BETI1 | 0.00 | 1.00 | 1.25 | 2.75 | 4.00 | 4.75 | 5.50 | 6.25 | 5.75 | 4.75 | 3.50 | 4.25 | 3.25 | 3.50 |
| | 1.0% | | | | | | | | | | | | | | |
| | IMI/Dermoval | 0.00 | 0.75 | 1.25 | 2.50 | 4.00 | 4.50 | 5.75 | 4.25 | 2.75 | 1.00 | 0.75 | 0.25 | 0.50 | 0.25 |
| | Tolerance | 0.00 | 0.00 | 0.00 | 0.00 | 0.00 | 0.00 | 0.00 | 0.00 | 0.00 | 0.00 | 0.00 | 0.00 | 0.00 | 0.00 |
| | BETi1 0.01% | | | | | | | | | | | | | | |
| | Tolerance | 0.00 | 0.00 | 0.00 | 0.00 | 0.00 | 0.00 | 0.00 | 0.00 | 0.00 | 0.00 | 0.00 | 0.00 | 0.00 | 0.00 |
| | BETi1 1.0% | | | | | | | | | | | | | | |

TABLE 1C
Change in Modified PASI Mean score values

| | 0 | 1 | 2 | 3 | 4 | 5 | 6 | 7 |
|---|---|---|---|---|---|---|---|---|
| IMI/Vehicle | 4.5 | 4.75 | 5 | 5.5 | 5.375 | 6 | 5.75 | 5.875 |
| IMI/ | 4.25 | 5.375 | 4.75 | 4.25 | 4 | 3.375 | 3 | 3 |
| BETi1 0.001% | | | | | | | | |
| IMI/ | 4.375 | 4 | 2.875 | 2.5 | 2.125 | 1 | 1.25 | 1 |
| BETi1 0.01% | | | | | | | | |
| IMI | 4.375 | 4.375 | 3.125 | 2 | 0.75 | 0.375 | 0.25 | 0.25 |
| BETi1 0.1% | | | | | | | | |
| IMI/Dermoval | 4.5 | 3.125 | 2 | 0.625 | 0.375 | 0.125 | 0.25 | 0.125 |

TABLE 1D
Change in Modified PASI Mean score values

| | 0 | 1 | 2 | 3 | 4 | 5 | 6 | 7 |
|---|---|---|---|---|---|---|---|---|
| Vehicle | 100 | 105.56 | 111.11 | 122.22 | 119.44 | 133.33 | 127.78 | 130.56 |
| BETi1 0.001% | 100 | 126.47 | 111.76 | 100 | 94.12 | 79.41 | 70.59 | 70.59 |
| BETi1 0.01% | 100 | 91.43 | 65.71 | 57.14 | 48.57 | 22.86 | 28.57 | 22.86 |
| BETi1 0.1% | 100 | 100 | 71.43 | 45.72 | 17.142 | 8.57 | 5.71 | 5.71 |
| Dermoval | 100 | 69.44 | 44.44 | 13.89 | 8.33 | 2.78 | 5.56 | 2.78 |

TABLE 2A
Body weight.

| | | D 1 | D 3 | D 5 | D 7 | D 8 | D 9 | D 10 | D 11 | D 12 | D 13 | D 14 |
|---|---|---|---|---|---|---|---|---|---|---|---|---|
| Mean | EXP/Vehicle | 18.43 | 18.28 | 17.96 | 18.42 | 18.38 | 18.69 | 18.59 | 18.44 | 18.77 | 19.21 | 19.18 |
| body | IMI/Vehicle | 17.83 | 16.39 | 16.13 | 17.06 | 16.88 | 16.62 | 16.66 | 16.39 | 16.51 | 16.76 | 16.91 |
| weights | IMI/- BETi1 | 17.86 | 16.49 | 16.28 | 16.70 | 16.75 | 16.91 | 16.99 | 16.91 | 16.96 | 17.31 | 18.26 |
| | 0.001% | | | | | | | | | | | |
| | IMI/BETi1 | 18.38 | 16.43 | 17.15 | 17.98 | 17.61 | 17.86 | 17.85 | 17.93 | 18.18 | 18.80 | 18.24 |
| | 0.01% | | | | | | | | | | | |
| | IMI/BETi1 | 18.35 | 16.78 | 16.85 | 17.10 | 16.88 | 17.38 | 17.62 | 17.45 | 17.63 | 18.06 | 18.25 |
| | 0.1% | | | | | | | | | | | |
| | IMI/- BETi1 | 18.11 | 16.63 | 17.15 | 17.53 | 17.80 | 17.38 | 16.76 | 17.08 | 17.11 | 17.54 | 17.30 |
| | 1.0% | | | | | | | | | | | |
| | IMI/Dermoval | 18.39 | 16.36 | 16.74 | 17.49 | 17.07 | 16.47 | 15.75 | 15.57 | 15.70 | 15.74 | 15.71 |
| | Tolerance | 17.78 | 17.85 | 18.38 | 18.35 | 18.57 | 18.72 | 18.71 | 18.34 | 19.07 | 19.17 | 19.31 |
| | BETi1 0.01% | | | | | | | | | | | |
| | Tolerance | 18.19 | 18.17 | 18.95 | 19.10 | 18.61 | 18.73 | 18.89 | 18.97 | 19.63 | 19.63 | 19.60 |
| | BETi1 1.0% | | | | | | | | | | | |

TABLE 2B
Absolute Body Weight Change from day 1 to day 14

| | Mouse | Mouse | | | | |
|---|---|---|---|---|---|---|
| Cage | cage no. | study no. | Treatment | D 1 | D 14 | D14 − D1 |
| 1 | 1 | 1 | EXP/Vehicle | 16.81 | 17.08 | 0.27 |
| | 2 | 2 | | 18.64 | 19.60 | 0.96 |
| 2 | 1 | 3 | | 19.22 | 19.15 | −0.07 |
| | 2 | 4 | | 19.06 | 20.87 | 1.81 |
| 3 | 1 | 5 | IMI/-Vehicle | 19.66 | 17.50 | −2.16 |
| | 2 | 6 | | 19.16 | 20.45 | 1.29 |
| 4 | 1 | 7 | | 17.78 | 15.77 | −2.01 |
| | 2 | 8 | | 14.72 | 13.93 | −0.79 |
| 5 | 1 | 9 | IMI/ | 19.49 | 17.74 | −1.75 |
| | 2 | 10 | BETi1 -0.001% | 15.61 | 17.19 | 1.58 |
| 6 | 1 | 11 | | 18.15 | 18.37 | 0.22 |
| | 2 | 12 | | 18.19 | 19.74 | 1.55 |
| 7 | 1 | 13 | IMI/ | 18.32 | 17.71 | −0.61 |
| | 2 | 14 | BETi1 -0.01% | 18.19 | 17.69 | −0.50 |
| 8 | 1 | 15 | | 18.60 | 19.65 | 1.05 |
| | 2 | 16 | | 18.41 | 17.89 | −0.52 |
| 9 | 1 | 17 | IMI/ | 17.66 | 16.42 | −1.24 |
| | 2 | 18 | BETi1 0.1% | 19.86 | 19.62 | −0.24 |
| 10 | 1 | 19 | | 18.46 | 18.98 | 0.52 |
| | 2 | 20 | | 17.40 | 17.97 | 0.57 |
| 11 | 1 | 21 | IMI/ | 18.33 | 17.88 | −0.45 |
| | 2 | 22 | BETi1 1.0% | 18.78 | 18.69 | −0.09 |
| 12 | 1 | 23 | | 17.16 | 16.53 | −0.63 |
| | 2 | 24 | | 18.18 | 16.11 | −2.07 |
| 13 | 1 | 25 | IMI/ | 20.65 | 16.55 | −4.10 |
| | 2 | 26 | Dermoval | 16.48 | 13.80 | −2.68 |
| 14 | 1 | 27 | | 17.98 | 15.93 | −2.05 |
| | 2 | 28 | | 18.44 | 16.55 | −1.89 |
| 15 | 1 | 29 | Tolerance | 17.83 | 20.26 | 2.43 |
| | 2 | 30 | BETi1_0.01% | 17.60 | 19.15 | 1.55 |
| 16 | 1 | 31 | | 18.92 | 19.62 | 0.70 |
| | 2 | 32 | | 16.78 | 18.20 | 1.42 |
| 17 | 1 | 33 | Tolerance | 19.18 | 20.00 | 0.82 |
| | 2 | 34 | BETi1 1.0% | 18.40 | 19.58 | 1.18 |
| 18 | 1 | 35 | | 18.49 | 20.57 | 2.08 |
| | 2 | 36 | | 16.70 | 18.23 | 1.53 |

TABLE 2C
Mean percent change in body weight (body weight on each treatment
day compared to first day of treatment on Day 7)

| | D 7 | D 8 | D 9 | D 10 | D 11 | D 12 | D 13 | D 14 |
|---|---|---|---|---|---|---|---|---|
| Weight gm | | | | | | | | |
| EXP/Vehicle | 18.42 | 18.38 | 18.69 | 18.59 | 18.44 | 18.77 | 19.21 | 19.18 |
| IMI/Vehicle | 17.06 | 16.88 | 16.62 | 16.66 | 16.39 | 16.51 | 16.76 | 16.91 |
| IMI/BETi1 0.1% | 17.10 | 16.88 | 17.38 | 17.62 | 17.45 | 17.63 | 18.06 | 18.25 |
| IMI/Dermoval 0.05% | 17.49 | 17.07 | 16.47 | 15.75 | 15.57 | 15.70 | 15.74 | 15.71 |
| % Change from D 0 | | | | | | | | |
| EXP/Vehicle | 100 | 99.78 | 101.47 | 100.89 | 100.08 | 101.88 | 104.27 | 104.09 |
| IMI/Vehicle | 100 | 99 | 97 | 98 | 96 | 97 | 98 | 99 |
| IMI/BETi1 0.1% | 100 | 99 | 102 | 103 | 102 | 103 | 106 | 107 |
| IMI/Dermoval 0.05% | 100 | 98 | 94 | 90 | 89 | 90 | 90 | 90 |

TABLE 3
Use of Enrichment

| | | Before | | | | | | | | | | | | | | D 1- | D 8- |
|---|---|---|---|---|---|---|---|---|---|---|---|---|---|---|---|---|---|
| | | D 1 | D 2 | D 3 | D 4 | D 5 | D 6 | D 7 | D 8 | D 9 | D 10 | D 11 | D 12 | D 13 | D 14 | D 8 | D 14 |
| Mean | EXP/ | 4.00 | 2.25 | 4.00 | 4.00 | 2.00 | 4.00 | 4.00 | 4.00 | 3.75 | 4.00 | 4.00 | 3.50 | 4.00 | 4.00 | 3.46 | 3.88 |
| score | Vehicle | | | | | | | | | | | | | | | | |
| use | IMI/ | 3.75 | 0.00 | 0.25 | 0.75 | 1.00 | 1.25 | 3.00 | 4.00 | 0.25 | 1.00 | 2.50 | 1.50 | 3.00 | 4.00 | 1.46 | 2.04 |
| enrichment | Vehicle | | | | | | | | | | | | | | | | |
| | IMI/BETi | 4.00 | 0.25 | 0.75 | 1.00 | 1.25 | 1.25 | 4.00 | 4.00 | 1.00 | 2.25 | 3.75 | 4.00 | 4.00 | 4.00 | 1.79 | 3.17 |
| | 0.001% | | | | | | | | | | | | | | | | |
| | IMI/BETi | 4.00 | 0.50 | 0.50 | 0.75 | 0.75 | 1.25 | 3.50 | 4.00 | 2.25 | 3.25 | 4.00 | 4.00 | 4.00 | 4.00 | 1.61 | 3.58 |
| | 0.01% | | | | | | | | | | | | | | | | |
| | IMI/-BETi | 4.00 | 0.75 | 1.25 | 1.25 | 0.50 | 0.75 | 3.00 | 3.25 | 2.75 | 3.75 | 4.00 | 4.00 | 4.00 | 4.00 | 1.54 | 3.75 |
| | 0.1% | | | | | | | | | | | | | | | | |
| | IMI/BETi | 3.75 | 0.75 | 1.00 | 1.00 | 1.50 | 1.50 | 2.00 | 2.25 | 2.00 | 3.00 | 3.50 | 2.25 | 2.75 | 4.00 | 2.56 | 3.58 |
| | 1.0% | | | | | | | | | | | | | | | | |
| | IMI/ | 4.00 | 1.75 | 1.75 | 1.75 | 0.50 | 1.50 | 3.50 | 4.00 | 3.25 | 4.00 | 4.00 | 2.75 | 4.00 | 4.00 | 2.56 | 3.58 |
| | Dermoval | | | | | | | | | | | | | | | | |
| | Tolerance | 4.00 | 3.00 | 4.00 | 4.00 | 2.00 | 4.00 | 4.00 | 4.00 | 3.75 | 4.00 | 4.00 | 3.00 | 4.00 | 4.00 | 2.71 | 3.57 |
| | BETi 0.01% | | | | | | | | | | | | | | | | |
| | Tolerance | 4.00 | 3.75 | 4.00 | 4.00 | 2.50 | 4.00 | 4.00 | 4.00 | 3.50 | 4.00 | 4.00 | 4.00 | 4.00 | 4.00 | 3.46 | 3.88 |
| | BETi 1.0% | | | | | | | | | | | | | | | | |

TABLE 4
7TEWL Scores

| | | Upper | Mid | Lower | |
|---|---|---|---|---|---|
| | | back | back | back | Mean |
| Mean | EXP/Vehicle | 8.04 | 8.18 | 8.54 | 8.25 |
| TEWL | IMI/Vehicle | 63.45 | 69.35 | 55.34 | 62.71 |
| score | IMI/BETi10.001% | 37.67 | 41.26 | 42.11 | 40.35 |
| values | IMI/BETi10.01% | 26.87 | 34.53 | 57.25 | 39.55 |
| (g/h/m2) | IMI/BETi10.1% | 18.11 | 24.04 | 39.27 | 27.14 |
| | IMI/1.0% | 44.36 | 40.80 | 30.63 | 38.60 |
| | IMI/Dermoval | 12.30 | 16.61 | 16.75 | 15.22 |
| | Tolerance BETi10.01% | 8.82 | 8.93 | 8.44 | 8.73 |
| | Tolerance BETi11.0% | 11.85 | 9.27 | 8.87 | 10.00 |

TABLE 5
Inflammatory Biomarkers

| | | IL-1β | IL-6 | IL-8 | IL-17 | IL-18 | IL-23 | IL-36 | TNF-α |
|---|---|---|---|---|---|---|---|---|---|
| Mean | EXP/Vehicle | 7.84 | 13.75 | 4.04 | 15.27 | 41.27 | 11.97 | 6.88 | 7.72 |
| score | IMI/Vehicle | 54.29 | 129.99 | 46.15 | 98.79 | 280.15 | 56.15 | 72.35 | 43.04 |
| values | IMI/BETi1 0.001% | 49.00 | 123.25 | 41.82 | 74.77 | 241.77 | 51.02 | 65.63 | 36.20 |
| (pg/mL) | IMI/BETi10.01% | 32.62 | 74.31 | 30.82 | 33.44 | 146.86 | 30.04 | 30.38 | 22.16 |
| | IMI/BETi1 0.1% | 24.31 | 56.29 | 21.40 | 25.08 | 138.90 | 20.66 | 26.91 | 18.27 |
| | IMI/BETi1 1.0% | 39.46 | 96.09 | 23.50 | 54.13 | 202.85 | 33.19 | 52.58 | 25.05 |
| | IMI/Dermoval | 14.59 | 16.13 | 7.82 | 14.68 | 49.82 | 13.67 | 17.60 | 7.20 |

### Example 2. In-Vivo Incisional Skin Wound Model Induced in Female Hairless Mice for Testing Formulations with Active Agent for Wound Healing

To evaluate the effects of the formulation containing the BETi1 on the rate of wound healing, the formulation was topically applied on incisional wounds induced in female Hairless mice in accordance with the protocol set out in Experimental Method B.

The effect of treatment with the formulation containing the BETi1 at the highest concentration 1% was compared to the effect obtained with vehicle (negative control) and hydroalcoholic gels (positive control) which are known to inhibit skin healing.

BETi1 demonstrates improvement in reducing fibrotic tissue mass and overall skin repair outcomes as described below.

As early as treatment day 5, animals treated with BETi1 1% had a statistically significant decrease (improvement) in global external lesion severity score, a comprehensive evaluation of length, width, swelling and visibility of lesions, compared to those treated with hydroalcoholic gel beginning at treatment day 5. 1% BETi1 formulation had no significant effect on body weight gain between treatment groups (see Table 6A and 6B). 1% BETi1 had no significant effect on width or length of a scar compared to hydroalcoholic gel. A significant difference in the length of the wound treated with either vehicle or 1% BETi1 from day 5 and the width of the wound on day 5 and from Day 9 compared to hydroalcoholic gel was observed (Table 7 and Table 8 respectively).

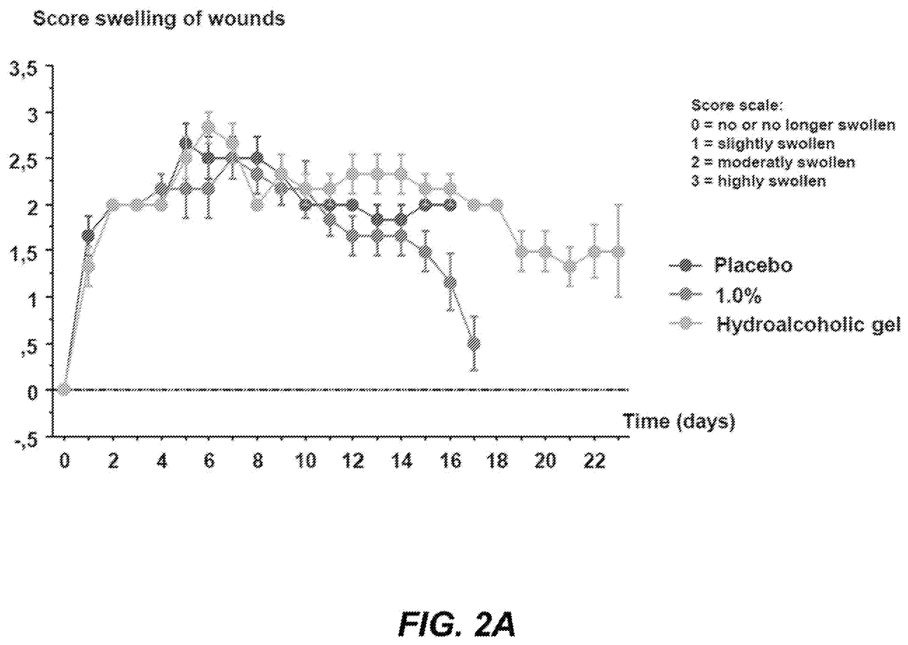

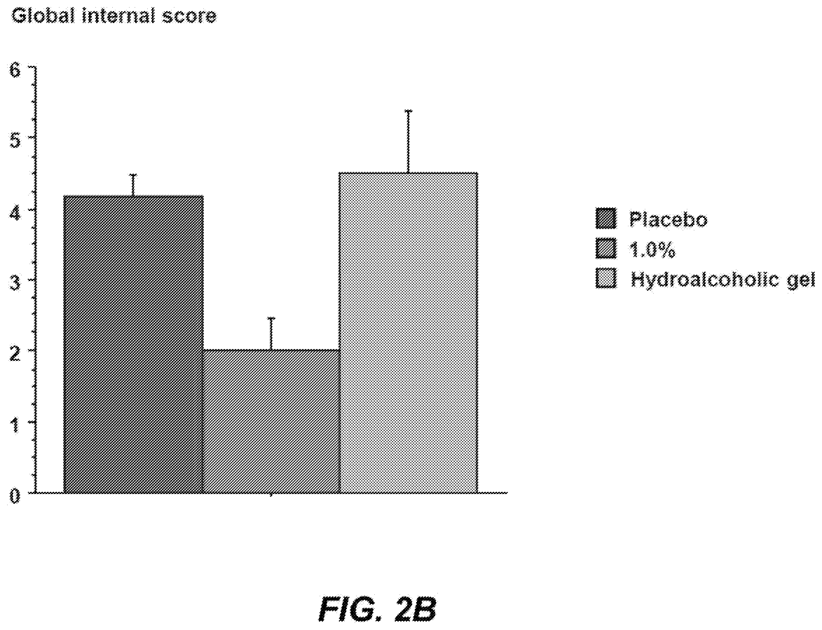

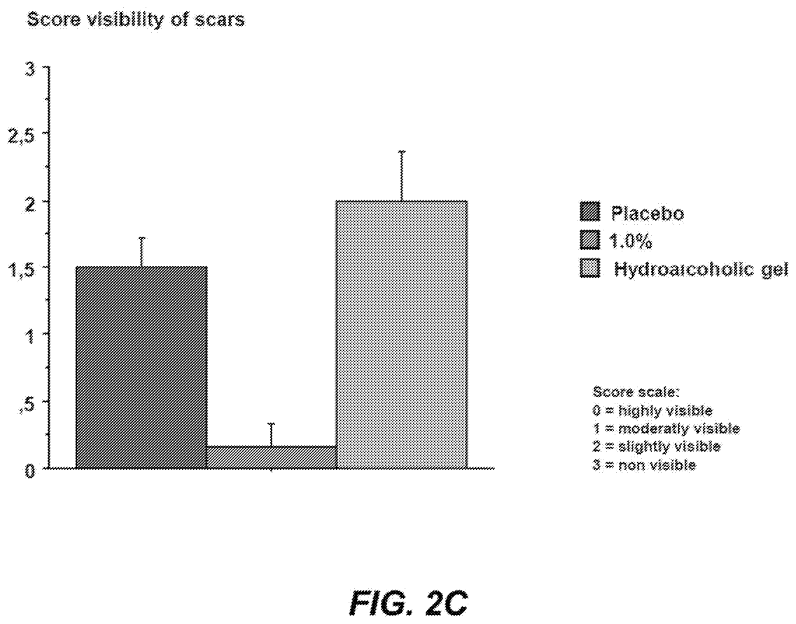

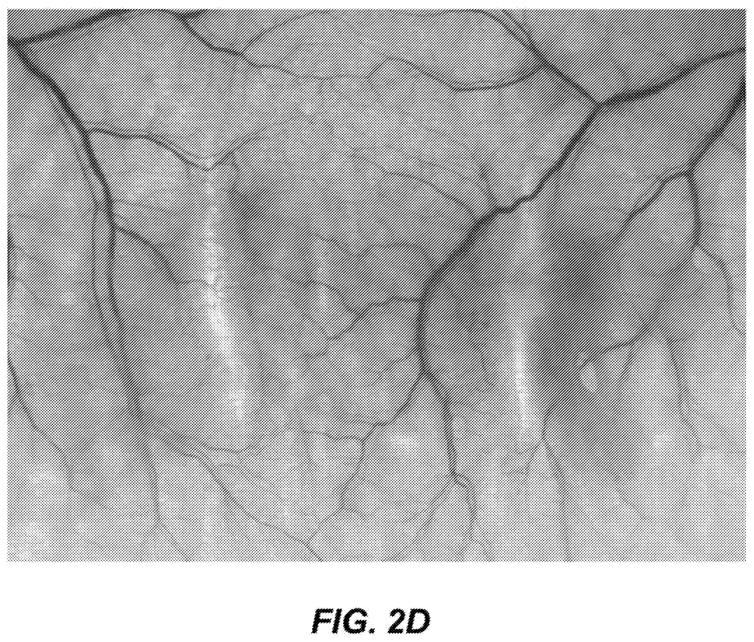

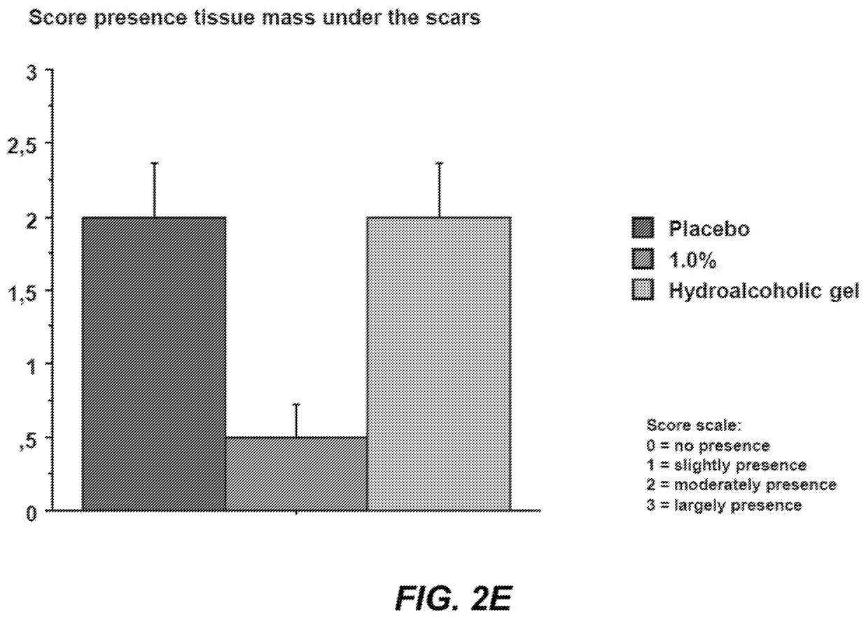

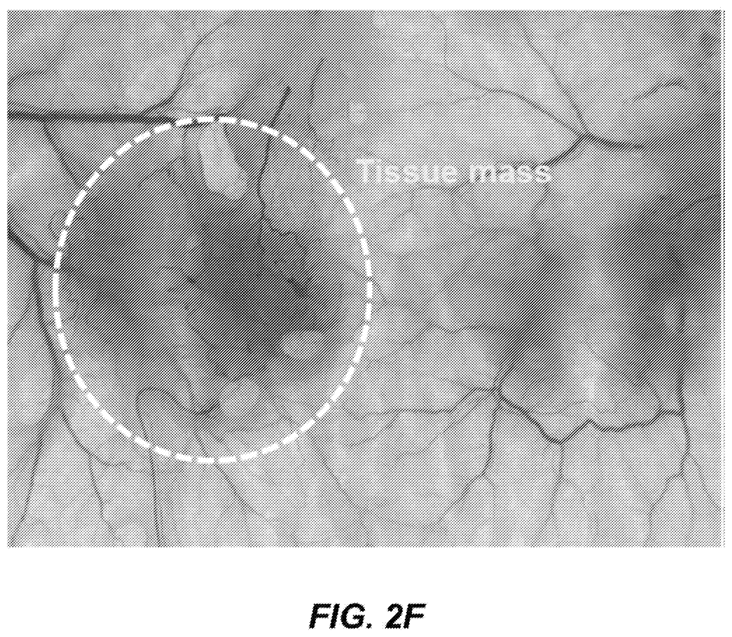

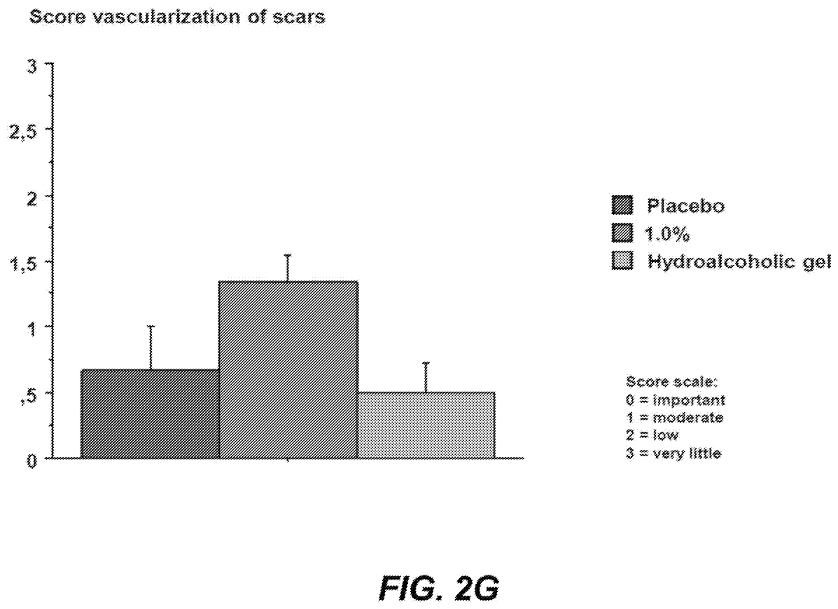

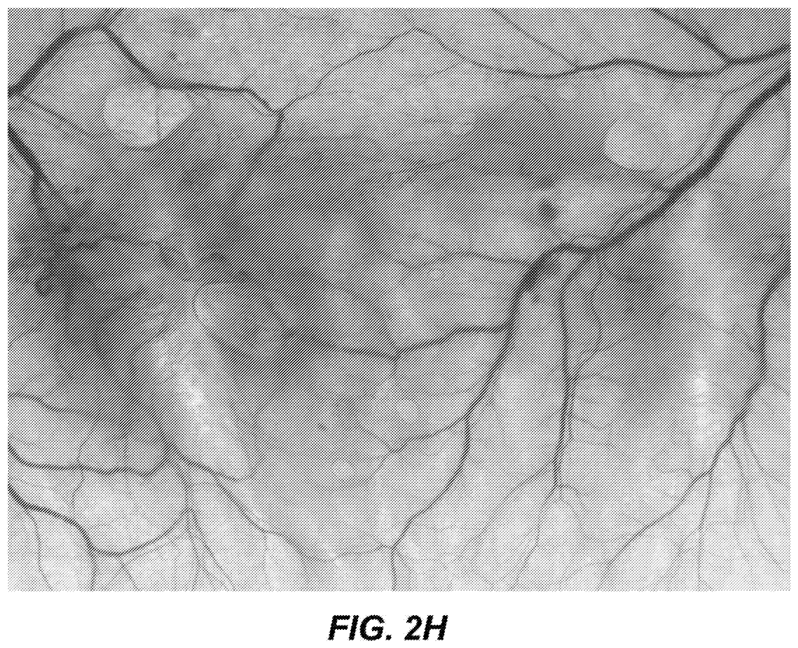

Surprisingly, treatment of skin wounds with the 1% BETi1 formulation, rapidly reduced the swelling at wound edges and said reduction was significantly different on Days 15, 16 and 17 compared to vehicle and hydroalcoholic gel. Reduction of swelling resulted in reduced presence of tissue mass under the scar (Table 9 and FIG. 2A). Without being bound by any theory, reduction in swelling and tissue mass may be attributed to the anti-inflammatory effect of 1% BETi1.

1% BETi1 formulation had no significant effect on visibility of wounds compared to hydroalcoholic gel. But from Day 12 a significant difference was observed in the visibility of the wound treated with either vehicle or 1% BETi1 compared to hydroalcoholic gel (Table 10).

1% BETi1 formulation had no significant effect on the global external score (the sum of the scores of the length, width, swelling and visibility of wounds) of the wound compared to hydroalcoholic gel. However, a significant difference from day 5 (except for D8) in the global external score was observed for wounds treated with either vehicle or 1% BETi1 formulation compared to hydroalcoholic gel (Table 11).

Treatment of skin wounds with 1% BETi1 did not promote or delay the complete healing of skin wounds compared to vehicle, but both vehicle and 1% were significantly better than hydroalcoholic gel (Table 12). Lesions treated with BETi1 1% completely healed (mean time to heal: 15.5 days) approximately 5 days earlier compared to those treated with hydroalcoholic gel (mean time to heal: 21 days).

Surprisingly, 1% BETi1 formulation had significant effect on the global internal score as assessed by macroscopic internal scoring compared to vehicle (P<0.01) and hydroalcoholic gel (P<0.05) (Table 13 and FIG. 2B). The global internal score is the sum of visibility of scars score, presence of tissue mass under the scar and vascularization. Animals treated with BETi1 1% had improved internal scar visibility when compared to either vehicle or hydroalcoholic gel. 1% BETi1 formulation had significant effect on the visibility of scars score as shown in FIG. 2C and Table 13 compared to vehicle and hydroalcoholic gel (P<0.005) and as shown in pictorial representation thereof (FIG. 2D). In other words, animals treated with BETi1 has a significant lower global internal lesion severity score than those treated with vehicle or hydroalcoholic gel. This indicates an improved internal lesion outcome and a positive effect on reducing the formation of fibrotic tissue mass in the lesion bed. Correspondingly, animals treated with BETi1 1% had less tissue mass/fibrosis under the scar compared to either vehicle or hydroalcoholic gel (P<0.05) as shown in FIG. 2E and Table 13 and pictorial representation thereof (FIG. 2F). Animals treated with BETi1 1% had numerically higher degree of wound vascularization when compared to either vehicle or hydroalcoholic gel (not statistically significant) as shown FIG. 2G and Table 13 compared to vehicle and hydroalcoholic gel and pictorial representation thereof (FIG. 2H). By the end of treatment, healed lesions treated with BETi1 1% appeared less visually distinct, more macular in nature with an improved overall aesthetic outcome compared to the other treatments. BETi1 vehicle and BETi1 1% appeared to be well-tolerated both within the lesion sites, based on the absence of irritation, and in general throughout the treatment period.

Overall, excisions treated with BETi 1% had a lower global internal score compared to either vehicle or hydroalcoholic gel indicative of an improved scar outcome. As shown in FIG. 2I generally, scars of mice treated with 1% BETi1 formulation appeared more aesthetic, flatter and less visible which could be due to a faster re-modelling of skin tissue. They exhibited little evidence of residual swelling. In wound treated with vehicle there was still evidence of minor swelling around incision sites. Wounds treated with hydroalcoholic gel moderate swelling was clearly evident at end of treatment. Although healed, residual scabbing still remained, and incision sites were clearly visible.

Without being bound by any theory it is postulated that wound healing properties of the BETi 1 formulation may improve healing of PG wounds without scarring.

TABLE 6A

| Mean body weight | | | | | | | | | | | | |
|---|---|---|---|---|---|---|---|---|---|---|---|---|
| (gram) per day | | D 0 | D 2 | D 4 | D 7 | D 9 | D 11 | D 13 | D 16 | D 18 | D 20 | D 23 |
| Mean | Vehicle | 26.98 | 26.29 | 26.61 | 27.28 | 27.49 | 27.28 | 27.18 | 28.11 | — | — | — |
| body | 1% BETi1 | 26.61 | 25.53 | 25.70 | 26.10 | 26.85 | 27.01 | 26.88 | 27.33 | — | — | — |
| weights | Hydroalcoholic | 27.02 | 25.77 | 26.17 | 26.11 | 26.27 | 26.20 | 27.00 | 26.52 | 26.52 | 26.54 | 24.60 |
| | gel | | | | | | | | | | | |

TABLE 6B

| | Mouse | Mouse | | | | MBWC |
|---|---|---|---|---|---|---|
| Cage | cage no. | study no. | Treatment | D 0 | D 16 | D 16 − D 0 |
| 1 | 1 | 1 | Vehicle | 27.11 | 28.54 | 1.43 |
| | 2 | 2 | | 26.65 | 27.71 | 1.06 |
| | 3 | 3 | | 27.19 | 28.09 | 0.90 |
| 2 | 1 | 4 | BETi11.0% | 28.64 | 28.47 | −0.17 |
| | 2 | 5 | | 26.19 | 28.43 | 2.24 |
| | 3 | 6 | | 25.00 | 25.10 | 0.10 |
| 3 | 1 | 7 | Hydroalcoholic | 28.03 | 26.70 | −1.33 |
| | 2 | 8 | gel | 24.97 | 25.36 | 0.39 |
| | 3 | 9 | | 28.07 | 27.49 | −0.58 |

TABLE 7

| Mean score length | | | | | | | | | | | | |
|---|---|---|---|---|---|---|---|---|---|---|---|---|
| (mm) per day | | D 0 | D 1 | D 2 | D 3 | D 4 | D 5 | D 6 | D 7 | D 8 | D 9 | D 10 |
| Mean | Vehicle | 10.00 | 10.00 | 10.00 | 10.00 | 9.33 | 8.33 | 7.83 | 7.17 | 7.00 | 5.83 | 5.67 |
| score | 1% BETi1 | 10.00 | 10.00 | 10.00 | 10.00 | 9.33 | 7.50 | 6.50 | 5.67 | 5.67 | 5.50 | 4.83 |
| length | Hydro | 10.00 | 10.00 | 10.00 | 10.00 | 9.67 | 8.83 | 8.50 | 7.83 | 7.17 | 7.17 | 7.00 |
| (mm) | Alcoholic | | | | | | | | | | | |
| | gel | | | | | | | | | | | |

| Mean score length | | | | | | | | | | | | | | |
|---|---|---|---|---|---|---|---|---|---|---|---|---|---|---|
| (mm) per day | | D 11 | D 12 | D 13 | D 14 | D 15 | D 16 | D 17 | D 18 | D 19 | D 20 | D 21 | D 22 | D 23 |
| Mean | Vehicle | 4.50 | 3.17 | 2.00 | 1.50 | 0.67 | 0.00 | — | — | — | — | — | — | — |
| score | 1% BETi1 | 3.83 | 2.67 | 1.50 | 1.17 | 0.50 | 0.33 | 0.00 | — | — | — | — | — | — |
| length | Hydro | 6.17 | 5.33 | 4.33 | 3.67 | 3.67 | 3.33 | 2.50 | 1.67 | 0.83 | 0.67 | 0.33 | 0.25 | 0.00 |
| (mm) | Alcoholic | | | | | | | | | | | | | |
| | gel | | | | | | | | | | | | | |

TABLE 8

| Mean score width | | | | | | | | | | | | |
|---|---|---|---|---|---|---|---|---|---|---|---|---|
| (mm) per day | | D 0 | D 1 | D 2 | D 3 | D 4 | D 5 | D 6 | D 7 | D 8 | D 9 | D 10 |
| Mean | Vehicle | 2.00 | 1.83 | 2.50 | 3.00 | 3.00 | 2.83 | 2.83 | 2.83 | 2.50 | 2.17 | 2.17 |
| score | 1% BETi1 | 2.00 | 2.00 | 2.67 | 2.83 | 3.00 | 2.33 | 2.50 | 2.50 | 2.67 | 2.83 | 2.17 |
| width | Hydroalcoholic | 2.00 | 2.00 | 2.67 | 3.00 | 3.00 | 3.00 | 2.83 | 2.83 | 3.00 | 2.83 | 3.00 |
| | gel | | | | | | | | | | | |

| Mean score width | | | | | | | | | | | | | | |
|---|---|---|---|---|---|---|---|---|---|---|---|---|---|---|
| (mm) per day | | D 11 | D 12 | D 13 | D 14 | D 15 | D 16 | D 17 | D 18 | D 19 | D 20 | D 21 | D 22 | D 23 |
| Mean | Vehicle | 1.83 | 1.33 | 1.00 | 1.00 | 0.67 | 0.00 | — | — | — | — | — | — | — |
| score | 1% BETi1 | 2.00 | 1.50 | 0.83 | 0.83 | 0.50 | 0.33 | 0.00 | — | — | — | — | — | — |
| width | Hydroalcoholic | 3.00 | 2.83 | 2.50 | 2.33 | 2.00 | 1.50 | 1.00 | 1.00 | 0.83 | 0.67 | 0.33 | 0.25 | 0.00 |
| | gel | | | | | | | | | | | | | |

TABLE 9

| Mean score swelling | | | | | | | | | | | | |
|---|---|---|---|---|---|---|---|---|---|---|---|---|
| per day | | D 0 | D 1 | D 2 | D 3 | D 4 | D 5 | D 6 | D 7 | D 8 | D 9 | D 10 |
| Mean | Vehicle | 0.00 | 1.67 | 2.00 | 2.00 | 2.00 | 2.67 | 2.50 | 2.50 | 2.50 | 2.33 | 2.00 |
| score | 1% BETi1 | 0.00 | 1.33 | 2.00 | 2.00 | 2.17 | 2.17 | 2.17 | 2.50 | 2.33 | 2.17 | 2.17 |
| swelling | Hydroalcoholic | 0.00 | 1.33 | 2.00 | 2.00 | 2.00 | 2.50 | 2.83 | 2.67 | 2.00 | 2.33 | 2.17 |
| | gel | | | | | | | | | | | |

| Mean score swelling | | | | | | | | | | | | | | |
|---|---|---|---|---|---|---|---|---|---|---|---|---|---|---|
| per day | | D 11 | D 12 | D 13 | D 14 | D 15 | D 16 | D 17 | D 18 | D 19 | D 20 | D 21 | D 22 | D 23 |
| Mean | Vehicle | 2.00 | 2.00 | 1.83 | 1.83 | 2.00 | 2.00 | — | — | — | — | — | — | — |
| score | 1% BETi1 | 1.83 | 1.67 | 1.67 | 1.67 | 1.50 | 1.17 | 0.50 | — | — | — | — | — | — |
| swelling | Hydroalcoholic | 2.17 | 2.33 | 2.33 | 2.33 | 2.17 | 2.17 | 2.00 | 2.00 | 1.50 | 1.50 | 1.33 | 1.50 | 1.50 |
| | gel | | | | | | | | | | | | | |

TABLE 10

| Mean score visibility | | | | | | | | | | | | |
|---|---|---|---|---|---|---|---|---|---|---|---|---|
| (external) per day | | D 0 | D 1 | D 2 | D 3 | D 4 | D 5 | D 6 | D 7 | D 8 | D 9 | D 10 |
| Mean | Vehicle | 3.00 | 3.00 | 3.00 | 3.00 | 3.00 | 3.00 | 3.00 | 3.00 | 3.00 | 3.00 | 2.83 |
| score | 1% BETi1 | 3.00 | 3.00 | 3.00 | 3.00 | 3.00 | 3.00 | 3.00 | 3.00 | 2.83 | 2.83 | 2.83 |
| visibility | Hydroalcoholic | 3.00 | 3.00 | 3.00 | 3.00 | 3.00 | 3.00 | 3.00 | 3.00 | 3.00 | 3.00 | 3.00 |
| (external) | gel | | | | | | | | | | | |

| Mean score visibility | | | | | | | | | | | | | | |
|---|---|---|---|---|---|---|---|---|---|---|---|---|---|---|
| (external) per day | | D 11 | D 12 | D 13 | D 14 | D 15 | D 16 | D 17 | D 18 | D 19 | D 20 | D 21 | D 22 | D 23 |
| Mean | Vehicle | 2.83 | 2.00 | 1.50 | 1.00 | 0.67 | 0.00 | — | — | — | — | — | — | — |
| score | 1% BETi1 | 2.67 | 2.17 | 1.17 | 0.83 | 0.50 | 0.33 | 0.00 | — | — | — | — | — | — |
| visibility | Hydroalcoholic | 3.00 | 3.00 | 3.00 | 3.00 | 2.50 | 2.17 | 1.67 | 1.50 | 0.83 | 0.67 | 0.33 | 0.25 | 0.00 |
| (external) | gel | | | | | | | | | | | | | |

TABLE 11

| Global external score | | | | | | | | | | | | |
|---|---|---|---|---|---|---|---|---|---|---|---|---|
| per day | | D 0 | D 1 | D 2 | D 3 | D 4 | D 5 | D 6 | D 7 | D 8 | D 9 | D 10 |
| Mean | Vehicle | 15.00 | 16.50 | 17.50 | 18.00 | 17.33 | 16.83 | 16.17 | 15.50 | 15.00 | 13.33 | 12.67 |
| global | 1% BETi1 | 15.00 | 16.33 | 17.67 | 18.17 | 17.50 | 15.00 | 14.17 | 13.67 | 13.50 | 13.33 | 12.00 |
| external | Hydroalcoholic | 15.00 | 16.33 | 17.33 | 18.00 | 17.67 | 17.33 | 17.17 | 16.33 | 15.17 | 15.33 | 15.17 |
| score | gel | | | | | | | | | | | |

| Global external | | | | | | | | | | | | | | |
|---|---|---|---|---|---|---|---|---|---|---|---|---|---|---|
| score per day | | D 11 | D 12 | D 13 | D 14 | D 15 | D 16 | D 17 | D 18 | D 19 | D 20 | D 21 | D 22 | D 23 |
| Mean | Vehicle | 11.17 | 8.50 | 6.33 | 5.33 | 4.00 | 2.00 | — | — | — | — | — | — | — |
| global | 1% BETi1 | 10.33 | 8.00 | 5.17 | 4.50 | 3.00 | 2.17 | 0.50 | — | — | — | — | — | — |
| external | Hydroalcoholic | 14.33 | 13.50 | 12.17 | 11.33 | 10.33 | 9.17 | 7.17 | 6.17 | 4.00 | 3.50 | 2.33 | 2.25 | 1.50 |
| score | gel | | | | | | | | | | | | | |

TABLE 12

| | | Day for complete |
|---|---|---|
| Time complete healing | | healing |
| Mean time for complete | Vehicle | 15.67 |
| wound healing | 1% BETi1 | 15.50 |
| | Hydroalcoholic gel | 21.00 |

TABLE 13

| | | Score | Score | | |
|---|---|---|---|---|---|
| | | visi- | presence | Score | Global |
| | | bility | tissue | vascular- | internal |
| Internal scores | | scar | mass | ization | score |
| Mean | Vehicle | 1.50 | 2.00 | 0.67 | 4.17 |
| internal | 1% BETi1 | 0.17 | 0.50 | 1.33 | 2.00 |
| scores | Hydroalcoholic gel | 2.00 | 2.00 | 0.50 | 4.50 |

### Example 3. In Vitro Cytokine Release Data from Th17 and Th2 Stimulated Human Skin Explants

To evaluate the effects of formulations containing two BETi compounds on the level of expression of different cytokine genes involved in inflammatory cell signalling pathways, the formulations were tested using an ex vivo human skin pharmacodynamic (PD) model. The model used stimulated human skin explants which mimic dermal inflammatory diseases in accordance with the protocol set out in Experimental Method C.

The Th2 mediated responses (e.g., acute atopic dermatitis) and Th17 mediated responses (e.g., psoriasis) were assessed for BETi1 and BETia. Betamethasome valerate (steroid) and Ruxolitinib (JAK1/2 inhibitor) were also tested as a positive control. The current study evaluated the ability of each compound applied at different concentrations to inhibit the gene expression of biomarkers involved in the Th2 (IL4, IL13, IL31, CCL17, CCL26, CCL2) and Th17 (CXCL10, IL17α, IL22, IL36γ, CCL20, SerpinB4) inflammatory pathways.

The effect of treatment with the formulation containing the BETi1 and BETia were compared to the effect obtained with untreated induced skin (negative control) and skin treated with a steroid or a JAK inhibitor both known to reduce cytokine levels. Ruxolitinib inhibits dysregulated JAK signaling associated with myelofibrosis. JAK1 and JAK2 recruit signal transducers and activators of transcription (STATs) to cytokine receptors leading to modulation of gene expression.

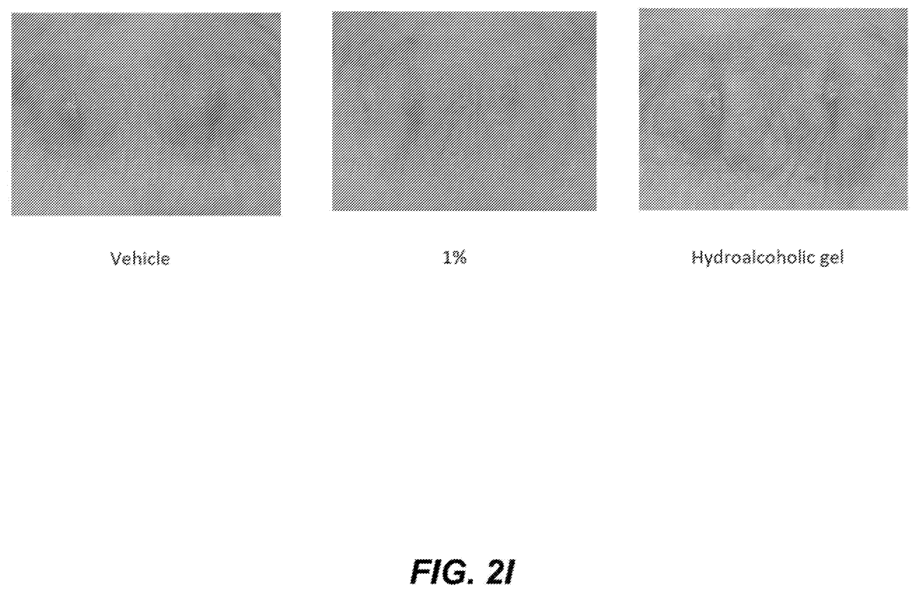

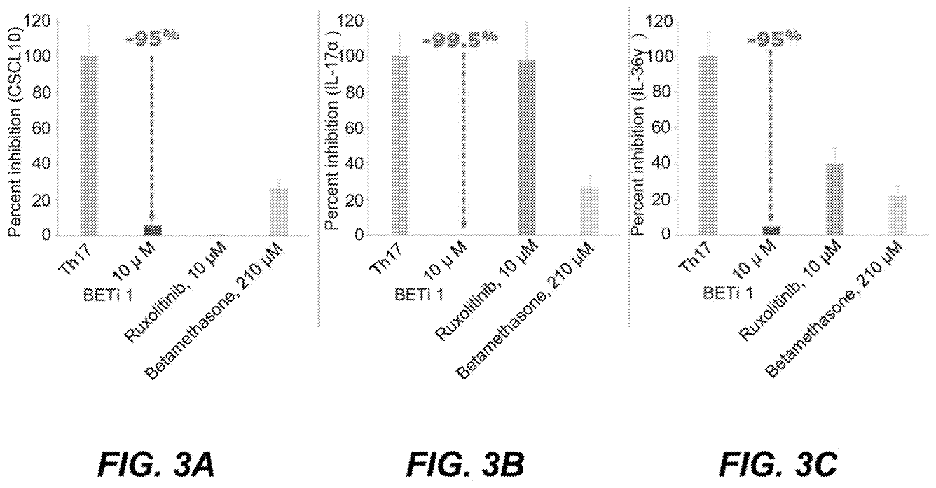

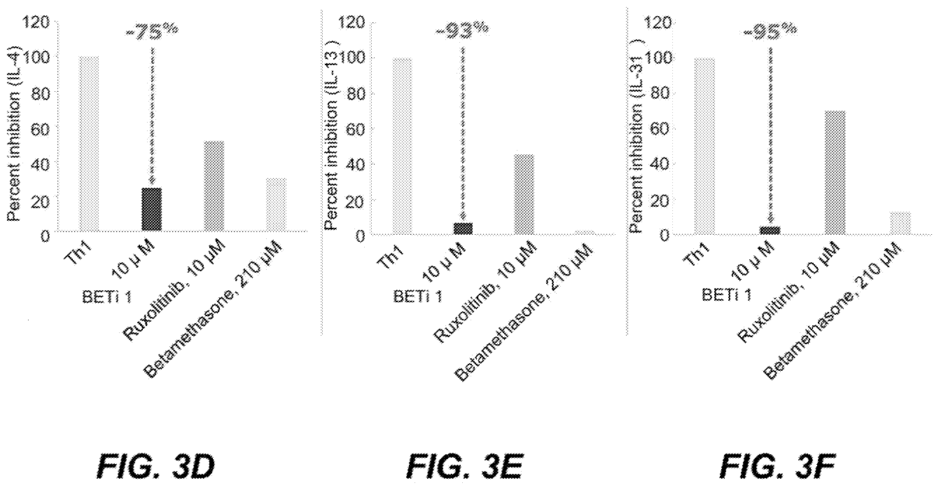

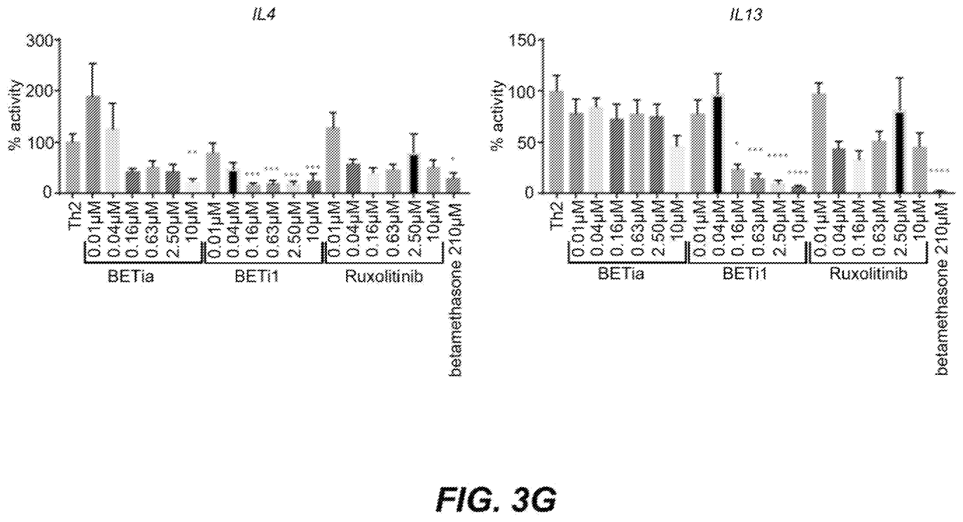

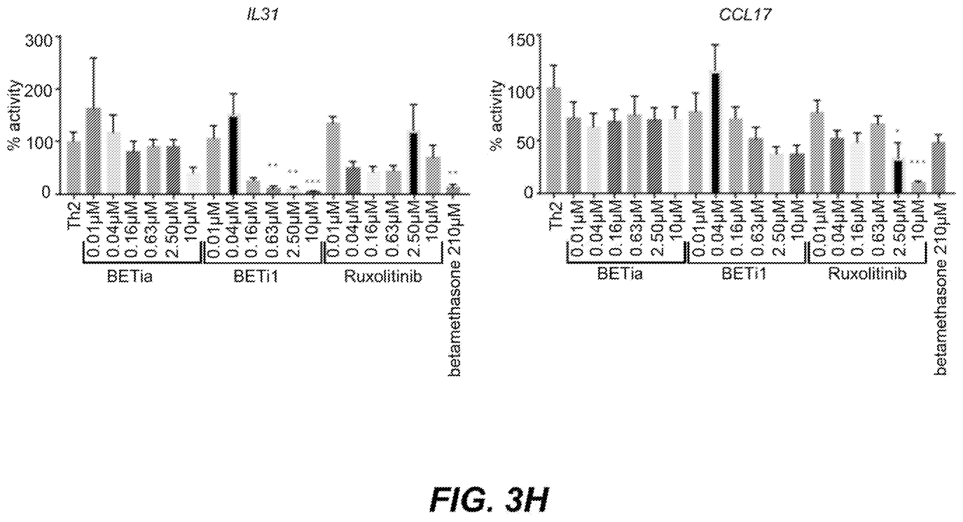

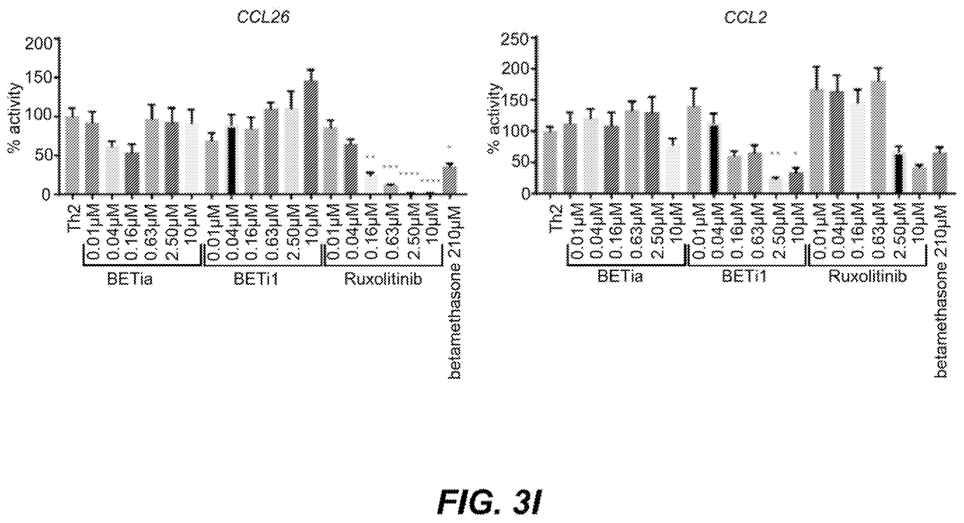

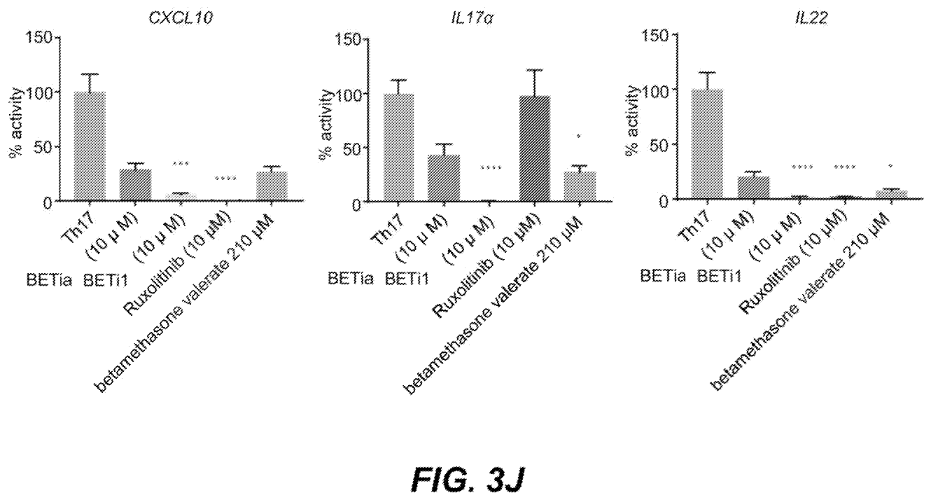

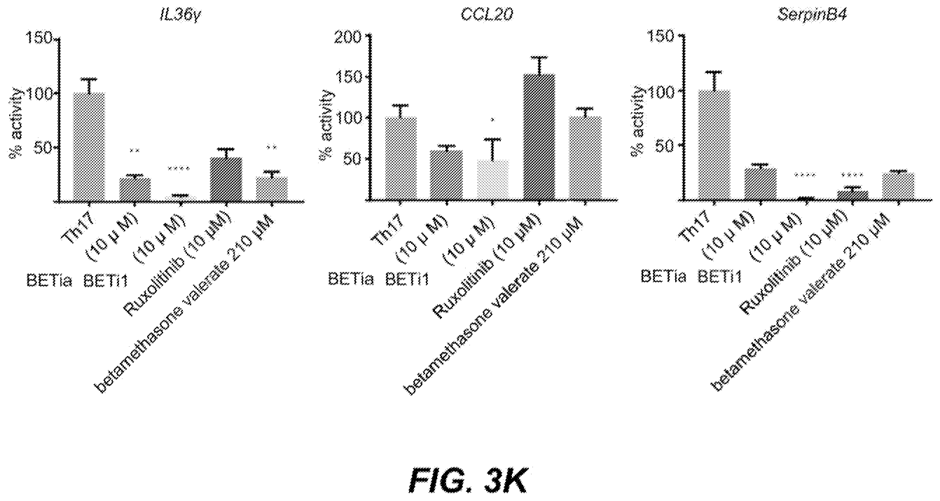

It has been surprisingly discovered that topical administration of BETi1 at a concentration of 10 μM significantly reduced expression of several key pro-inflammatory proteins relevant to Th17 (FIG. 3A-C) or TH2 (FIG. 3D-E)-mediated immune diseases (Table 14-17).

In the study, the release of several key pro-inflammatory cytokines was substantially reduced when Th17-stimulated human skin tissue was treated with BETi1, resulting in greater than 95% inhibition of IL-17, IL-36 and CXC motif chemokine ligand 10 (CXCL10) release relative to vehicle control. BETi1 also demonstrated a superior anti-inflammatory effect on these cytokines when compared to the ruxolitinib, at identical concentrations.

CXCL10, also known as LP-10, is an inflammatory cell chemoattractant secreted in response to interferon gamma. CXCL10 is significantly overexpressed in many immune diseases (>25-fold) verses untreated healthy skin. A 95% inhibition of CXCL10 (CXCL10)) expression, was demonstrated for skin treated with BETi1 as compared to untreated skin and said inhibition was significantly better than a steroid or Ruxolitinib (FIG. 3A).

T-cells are polarized to Th1 and Th17 cells the latter of which drives the production of IL17A which further upregulates the migratory action of pro-inflammatory cells and further inflammatory cell activation. A 99.5% inhibition of interleukin 17-alpha (IL17A) expression was demonstrated for skin treated with BETi1 as compared to untreated skin and said inhibition was significantly better than treatment with a steroid or JAK inhibitor (FIG. 3B).

IL36γ is implicated in upregulating IL-17A signaling-related genes and so able to amplify keratinocyte inflammatory responses by promoting not only their own expression but also that of other cytokines related to Th17 signaling. A 95% inhibition of interleukin IL36γ expression was demonstrated for skin treated with BETi1 as compared to untreated skin and said inhibition was significantly better than treatment with a steroid or JAK inhibitor (FIG. 3C).

IL-4 is secreted by several inflammatory cells and drives differentiation of naïve Th0 cells to Th2 cells in adaptive immunity. Along with IL-13 and their respective receptors, TL-4 is a significant contributor to allergenic response in diseases such as atopic dermatitis, rhinitis and allergenic asthma. A 75% inhibition of interleukin IL-4 expression was demonstrated for skin treated with BETi1 as compared to untreated skin and said inhibition was significantly better than treatment with a steroid or JAK inhibitor (FIG. 3D).

Similar to IL-4, IL-13 is an important contributor to the pathogenesis of allergenic diseases. IL-13 is significantly over-expressed in atopic dermatitis lesions and in airway epithelial cells in asthma patients. A 93% inhibition of interleukin IL-13 expression was demonstrated for skin treated with BETi1 as compared to untreated skin and said inhibition was significantly better than treatment JAK inhibitor and nearly comparable to treatment with a steroid (FIG. 3E).

IL-31 is overexpressed in several Th2-mediated inflammatory diseases. IL-31 has been implicated as having a direct role on pruritus signaling as the IL-31 receptor has been found in dorsal root ganglia that route transmission of sensory information to the spinal cord. A 95% inhibition of interleukin IL-31 expression was demonstrated for skin treated with BETi1 as compared to untreated skin and said inhibition was significantly better than treatment with a JAK inhibitor and nearly comparable to treatment with a steroid (FIG. 3F).

Percent inhibition of each biomarker calculated to the Th2 stimulated control is described in Table 14. Percent activity of IL4, IL13, TL31, CCL17, CCL26, and CCL2 is shown in FIG. 3G, FIG. 3H and FIG. 3I. For Th2-mediated stimulation, BETi1 resulted in the highest percent inhibition of IL4, IL13, IL31, and CCL2 (76-85%) gene expression compared to stimulated and untreated. Dose-dependent inhibition was observed with IL13, IL31, and CCL17. A dose-dependent increase was observed with CCL26 gene expression. Ruxolitinib at 10 μM resulted in the highest percent inhibition of CCL17 (ca. 90%) gene expression and a dose-dependent inhibition of CCL26.

Percent inhibition of each biomarker calculated to the Th17 stimulated control is described in Table 15. Percent activity of CXCL10, IL17α, and IL22 is shown in FIG. 3J and percent activity of IL36γ, CCL20, and serpinB4 is shown in FIG. 3K. With Th17-mediated stimulation, BETi1 resulted in the highest percent inhibition of IL17α, IL36γ, CCL20 and SerpinB4 gene expression compared to stimulated untreated skin. Ruxolitinib at 10 μM resulted in the highest percent inhibition of CXCL10 and IL22 (ca. 99%) gene expression and an increase in CCL20.

Overall, as shown in Table 16 and Table 17, BETi1 outperformed BETia in both the Th2 (e.g., atopic dermatitis) and Th17 (e.g., psoriasis) inflammatory human skin models for inhibition of inflammatory gene expression. Even so, in the light of its other properties including a selectivity for BD II, a lower liver clearance rate, and its impact on some cytokines BETia can in one or more embodiments be a useful candidate as a selective BET BDII inhibitor for systemic (e.g., oral) treatment of disorders.

TABLE 14
Percent inhibition following application of BETi1/BETia, Ruxolitnib, and betamethasone valerate
calculated to Th2 stimulated control (three skin donors; n = 3 per treatment per donor).

| | | | % inhibition | % inhibition | % Inhibition | % Inhibition | % Inhibition | % inhibition |
|---|---|---|---|---|---|---|---|---|
| Treatment | Concentration | | IL4 | IL13 | IL31 | CCL17 | CCL26 | CCL2 |
| BETia | 0.01 | μM | N/I | 22.0 | N/I | 28.2 | 8.3 | N/I |
| | 0.04 | μM | N/I | 15.8 | N/I | 2 [symbol] .8 | 38. [symbol] | N/I |
| | 0.16 | μM | 56.8 | 27.7 | 19.1 | 31.8 | 46.3 | N/I |
| | 0.63 | μM | 4 [symbol] .9 | 22.3 | [symbol] .4 | 25.4 | 2.8 | N/I |
| | 2.50 | μM | 57.5 | 24.9 | [symbol] .1 | 30.0 | 8.0 | N/I |
| | 10 | μM | 76. [symbol] | 53.4 | [symbol] . [symbol] | 2 [symbol] .2 | 8. [symbol] | 21.5 |
| BETi1 | | | 20.4 | 22.4 | N/I | 22.4 | 31.3 | N/I |
| | | | 51.8 | 3.0 | N/I | N/I | 10.9 | N/I |
| | | | 83.4 | 76.3 | 74. [symbol] | 2 [symbol] .0 | 15.2 | 40.0 |
| | | | 80. [symbol] | 84. [symbol] | 8 [symbol] .3 | 48.0 | N/I | 34.9 |
| | | | 82.0 | 90.2 | 8 [symbol] .7 | 62.3 | N/I | 76.5 |
| | | | 74.9 | 93.3 | 95.4 | 62. [symbol] | N/I | [symbol] .1 |
| Ruxolitinib | 0.01 | μM | N/I | 2.3 | N/I | 23.2 | 14.0 | N/I |
| | 0.04 | μM | 41.9 | 5 [symbol] .0 | 4 [symbol] .9 | 47.7 | 35.3 | N/I |
| | 0.16 | μM | [symbol] 0.4 | 66.3 | 57.9 | [symbol] 1.1 | 7 [symbol] .8 | N/I |
| | 0.63 | μM | 53.8 | 48.8 | 55.9 | 34.0 | 88.3 | N/I |
| | 2.50 | μM | 20.5 | 18.5 | N/I | [symbol] .4 | [symbol] 8.4 | 33.0 |
| | 10 | μM | 48.5 | 54. [symbol] | 29.9 | 89.9 | 98.5 | 57. [symbol] |
| Betamethasone | 210 | μM | 69. [symbol] | 97.8 | 87.4 | [symbol] 1.9 | 64.4 | 34. [symbol] |

N/I indicates no inhibition [symbol]
indicates data missing or illegible when filed

TABLE 15
Percent inhibition following application of BETi1/BETia, Ruxolitnib, and betamethasone valerate
calculated to Th17 stimulated control (three skin donors; n = 3 per treatment per donor).

| | | | % inhibition | % inhibition | % inhibition | % inhibition | % inhibition | % inhibition |
|---|---|---|---|---|---|---|---|---|
| Treatment | Concentration | | CXCL10 | IL17α | IL22 | IL36γ | CCL20 | Serpinβ4 |
| BETia | 10 | μM | 71.3 | [symbol] 7.2 | 79.7 | 78.2 | 40.1 | 71.2 |
| BETi1 | | | [symbol] 4.5 | 99.5 | [symbol] 8.3 | 9 [symbol] .3 | [symbol] 1. [symbol] | 98. [symbol] |
| Ruxolitinib | | | 99.8 | 2.8 | 98.5 | 5 [symbol] . [symbol] | N/I | 92.0 |
| Betamethasone | 210 | μM | 73. [symbol] | 73.1 | [symbol] 2. [symbol] | 77.7 | N/I | 7 [symbol] . [symbol] |

N/I indicates no inhibition. [symbol]
indicates data missing or illegible when filed

TABLE 16
Statistical analysis of samples treated with BETi1/BETia, ruxolitinib,
and betamethasone compared to Th2 stimulation using One-way ANOVA
with Dunnett's Multiple Comparisons Test (non-parametric).

| | | | P value | P value | P value | P value | P value | P value |
|---|---|---|---|---|---|---|---|---|
| Treatment | Concentration | | IL4 | IL13 | IL31 | CCL17 | CCL26 | CCL2 |
| BETia | 0.01 | μM | >0.9999 | >0.9999 | [symbol] | >0.9999 | >0.9999 | >0.9999 |
| | 0.04 | μM | >0.9999 | >0.9999 | >0.9999 | >0.9999 | >0.9999 | >0.9999 |
| | 0.15 | μM | 0.5545 | >0.9999 | >0.9999 | >0.9999 | [symbol] | >0.9999 |
| | 0.63 | μM | 0.7726 | >0.9999 | >0.9999 | >0.9999 | >0.9999 | >0.9999 |
| | 2.50 | μM | 0.1383 | >0.9999 | >0.9999 | >0.9999 | >0.9999 | >0.9999 |
| | 10 | μM | 0.0041 | 0.5554 | 0.9230 | >0.9999 | >0.9999 | >0.9999 |
| BETi1 | | | >0.9999 | >0.9999 | >0.9999 | >0.9999 | >0.9999 | >0.9999 |
| | | | [symbol] | [symbol] | >0.9999 | >0.9999 | >0.9999 | >0.9999 |
| | | | 0.0002 | 0.0106 | 0.1232 | >0.9999 | >0.9999 | >0.9999 |
| | | | 0.0007 | [symbol] | 0.0021 | >0.9999 | >0.9999 | >0.9999 |
| | | | 0.0005 | <0.0001 | 0.0022 | [symbol] | >0.9999 | 0.0028 |
| | | | 0.0005 | <0.0001 | 0.0002 | [symbol] | >0.9999 | 0.0214 |
| Ruxolitinib | 0.01 | μM | [symbol] | [symbol] | >0.9999 | >0.9999 | >0.9999 | >0.9999 |
| | 0.04 | μM | >0.9999 | 0.4297 | >0.9999 | >0.9999 | >0.9999 | >0.9999 |
| | 0.16 | μM | 0.1141 | [symbol] | >0.9999 | >0.9999 | 0.0077 | >0.9999 |
| | [symbol] | μM | [symbol] | [symbol] | [symbol] | [symbol] | 0.0008 | >0.9999 |
| | 2.50 | μM | [symbol] | >0.9999 | >0.9999 | 0.0275 | <0.0001 | >0.9999 |
| | 10 | μM | [symbol] | [symbol] | >0.9999 | 0.0002 | <0.0001 | 0.0731 |
| Betamethasone | 210 | μM | 0.0145 | <0.0001 | 0.0038 | >0.9999 | 0.0478 | >0.9999 |

[symbol]

indicates data missing or illegible when filed

TABLE 17
Statistical analysis of samples treated with BETi1/BETia, ruxolitinib, and betamethasone compared
to Th17 stimulation using One-way ANOVA with Dunnett's Multiple Comparisons Test (non-parametric).

| | | | % inhibition | % inhibition | % inhibition | % inhibition | % inhibition | % inhibition |
|---|---|---|---|---|---|---|---|---|
| Treatment | Concentration | | CXCL10 | IL17α | IL22 | IL36γ | CCL20 | Serpinβ4 |
| BETia | 10 | μM | 0.2193 | 0.1092 | 0.2909 | 0.0091 | 0.4571 | 0.1561 |
| BETi1 | | | 0.0005 | <0.0001 | <0.0001 | <0.0001 | 0.0241 | <0.0001 |
| Ruxolitinib | | | <0.0001 | [symbol] | <0.0001 | 0.2479 | 0.5842 | <0.0001 |
| Betamethasone | 210 | μM | 0.1703 | 0.0123 | 0.0123 | 0.0056 | [symbol] | 0.0586 |

[symbol]

indicates data missing or illegible when filed

The solubility of BETi was determined as described in Example 8 below.

### Section B:

### Experimental Method F: Formulation Preparation

Petrolatum is heated to approximately 70° C. in an appropriate beaker while being mixed. Petrolatum is fully melted to achieve clear liquid. API needed for preparation of the batch is placed in the beaker with preheated petrolatum and sonicated at approximately 70° C. for 30 minutes. The dispersion is then cooled to ambient temperature while being under overhead mixing.

Formulation of a hydrophobic carrier with multiple excipients can be achieved in a similar manner, where the hydrophobic excipients are heated to approximately 70° C. in an appropriate beaker while being mixed to achieve clear liquid. API needed for preparation of the batch is placed in the beaker with preheated excipients and sonicated or alternatively homogenized at approximately 70° C. for 30 minutes. The dispersion is then cooled to ambient temperature while being under overhead mixing. If a solution of API is desired the same procedure can be applied including one or more solvents in which the API has solubility (see e.g., Tables 39A and B). To the extent the excipients selected are liquid at a lower temperature the procedure can be carried out at that lower temperature until the API is completely dissolved.

Formulation of a hydrophilic carrier with API e.g., as a semi-solid carrier formulation in polyethylene glycol can be prepared as follows. The API is dissolved in low molecular weight polyethylene glycol e.g., PEG400 by mixing with overhead stirrer at ambient temperature. High molecular weight polyethylene glycol e.g., PEG3350 is heated to 70° C. while being mixed to generate clear melt. The solution of API in PEG400 is heated to approximately 60° C. and added to molten PEG3350 while being mixed by overhead stirrer and the mixture is cooled to 25° C. while being mixed.

### Experimental Method G: Chemical Stability

For Polarized Light Microscopy evaluation, the samples of the formulations in petrolatum are heated to 55° C. to melt most of the petrolatum crystals and make visible crystals of API. The API crystals are measured and photomicrographs are taken.

The microscopy samples of API are prepared by dispersing some (unmeasured amount) of API powder in a drop of mineral oil. The dispersion is done by a spatula. The concentration of API in mineral oil is unknown.

The stability of prepared formulations was assessed by HPLC testing of samples of two formulations, prepared with API from two different sources. One was exposed to 25° C. for 2.5 months and the other was exposed to 40° C. for 3 months. The area % of the main peak in the samples after temperature exposure was compared to area % of the main peak in initial samples of the same batches and in the corresponding APIs. The samples exposed to stability challenge were also evaluated for presence of new peaks, not observed in API or in initial samples.

Experimental Method H: Reconstituted Human Epidermis (RHE) Model Mimicking Vitiligo and Treatment with BETi1 Formulations and Vehicle

To generate the morphological and functional aspects of vitiligo, in vitro reconstituted epidermis with Normal Human Epidermal Keratinocytes (NHEK) and Normal Human Epidermal Melanocytes, darkly pigmented (NHEM-DP) are exposed to a cytokine cocktail (TNF-α and IFN-γ). The cytokine cocktail is applied during 48 hours in the culture medium of epidermis.

NHEK are isolated from the fore skin of 3 Caucasian donors, and NHEM-DP are isolated from 1 donor. The tissues are cultured at the air-liquid interface in Epilife medium (Fisher Scientific, M-EPI-500-A) containing supplements and antibiotics (Gentamycin, Fisher Scientific, 15710-049). They are maintained in a humid atmosphere at 37° C. with 5% CO2.

At day 9 of their reconstitution process, the culture medium is replaced with culture medium depleted in hydrocortisone (-HC). At this same stage of culture (day 9), the test formulations (BETi1 0.001%, 0.01%, 0.1%, 1%, and 1.5% ruxolitinib cream (OPZELURA, a JAK inhibitor) and corresponding vehicle are applied on the top of epidermis (on the stratum corneum) at 3 mg/cm².

After 24 hours of pre-treatment with the test formulations (at day 10), the epidermis is also stimulated with TNF-α and IFN-γ (both applied at 12 ng/ml in culture medium—HC) for additional 24 hours, so that test formulations are applied simultaneously with the cytokines on the stratum corneum of the epidermis during the same 24 hours.

After 24 hours of stimulation (at day 11) the treatments are stopped, and the culture medium is collected for MMP-9 and E-cadherin secretion quantification by ELISA. Three epidermis samples per condition are fixed in 4% formaldehyde solution and embedded in paraffin for further analysis (morphology analysis, melanin and melanocytes localisation/quantification). Three other epidermis samples per condition are put in a specific lysis buffer for total RNA extraction.

Experimental Method I: Morphological Analysis after H/E Staining of Histologic Sections of Paraffin-Embedded Epidermis

The tissue morphology is analyzed through a Hematoxylin/Eosin (H/E) staining. At the end of treatment, the tissues are fixed in 4% formaldehyde solution, dehydrated and embedded in paraffin. Sections of 6 m thick of tissue are generated using a microtome and laid over microscopic slides before staining with H/E used to visualize general tissue morphology. Slides are mounted with specific medium and examined with a Nikon Eclipse (Ni-E) photomicroscope coupled to a numerical camera (DS-Ri2).

Experimental Method J: Quantification of Secretion of MMP-9 and E-Cadherin by ELISA from Collected Culture Medium

At the end of the treatments, culture media is collected (from 3 epidermis per condition), aliquoted and stored at −20° C. until use in ELISA assays. The quantity of MMP-9 secreted by the epidermis at the end of the treatments is analysed using the human MMP-9 Quantikine ELISA kit (Bio-Techne; DMP900). The secretion of E-cadherin in culture media is measured using to human E-cadherin Quantikine ELISA kit (Bio-Techne; DCADE0) dedicated for the measurement of natural human E-cadherin in culture supernates, serum, plasma, urine, and saliva.

The ELISAs are performed following the instructions of the supplier. The quantities of MMP-9 and E-cadherin are determined by comparisons with standard curves performed with recombinant human MMP-9 or E-cadherin provided in the kits.

### Experimental Method K: Melanin Content and Localization Analysis by Fontana Masson Staining of RHE-MEL Sections

Melanin deposition on 6 μm paraffin sections of each epidermis (n=3) are highlighted through staining with Fontana Masson reagents (VWR—VWRK641295, VWRK641311; Klinipath—641215). Slides are mounted with specific medium and examined with a Nikon Eclipse (Ni-E) photomicroscope coupled to a numerical camera (DS-Ri2)

For the melanin quantification, three images of each epidermis are taken and analysed using the NIS-Element AR software (Nikon). For the quantification of melanin, a region of interest (ROI) is first defined and measured in the epidermis. This region includes only the basal layers. Then, two measures are taken by the software in this ROI. The first one represents the labelled surface on the ROI, and thus the area occupied by the melanin in the ROI. The second one corresponds to the global intensity of the staining in the ROI, reflecting a lighter or darker melanin. The relative quantification of melanin in the ROI is then performed by multiplying these two measures. To normalize the results to a given surface, this value was then divided by the surface of the ROI.

Experimental Method L: Melanocyte Localization after TRP1 Immunostaining

TRP-1 (tyrosinase related protein-1) is a melanocyte specific protein and a member of the tyrosinase gene family which also includes tyrosinase. It is very often used as specific marker of melanocytes. To visualize and quantify the melanocytes within the epidermis, sections of epidermis (6 μm thick) prepared from epidermis embedded in paraffin are used for TRP-1 immunostaining.

After deparaffination and re-hydration, antigen unmasking is performed by heating in citrate ph6 buffer. Samples are then saturated with goat serum, in order to avoid unspecific labelling.

Samples are incubated overnight, at 4° C., with a primary antibody raised against human TRP-1 (Abcam; Ab235447). After washing, the samples are incubated (for 30 minutes, at room temperature) with a secondary antibody anti-rabbit coupled to Alexa Fluor 488 dye (Thermo Fisher Scientific; A-11008). Samples are then incubated with DAPI or 4′,6′-diamidino-2-phenylindole (Thermo Fisher Scientific, D1306), a fluorescent molecule able of binding to the adenine and thymine bases of DNA and slides are mounted with specific medium, before taking the images. For each tissue/sample, 1 slide section is used to make 3 images. Images are taken using a Nikon Eclipse (Ni-E) photomicroscope coupled to a numerical camera (DS-Ri2). Quantification of the protein is performed with NIS-Element AR software (Nikon). The detection thresholds are firstly determined, using the most marked slices, in order to avoid saturation of detection. For each image, a region of interest (ROI) is delimited manually (including only the basal layers of epidermis which are supposed to contain the melanocytes) and its surface was measured. The software then measures the marked area within the delimited area, in number of pixels. The mean intensity of the marked surface multiplied by the “marked surface” determines the staining for the given ROI. This value is then normalized for a given surface by dividing the staining value by the surface of the ROI.

### Experimental Method M: Gene Expression Analysis

At the end of the treatments, total RNAs is extracted from 3 epidermis per condition (n=3) using the Qiagen RNeasy kit (Qiagen; 74106). Epidermis is rinsed with cold PBS and lysed in the ad hoc buffer provided in the kit. Extraction is performed according to the supplier's recommendations. The collected RNAs is stored at −80° C.

The RNA concentration is determined by spectrophotometric measurement (QIAxpert, Qiagen) and the RNA quality is analyzed by capillarity electrophoresis (Agilent Bioanalyzer 2100-Agilent RNA 6000 Nano Kit, 5067-1511). The integrity of the total RNA is assessed by visualization of intact ribosomal RNA bands. For the total RNA from higher eukaryotes, the size of the ribosomal bands should be 1.9 kb for the 18S-RNA and 4.7 kb for the 28S-RNA. The intensity of the band corresponding to the 28S RNA must be greater than the intensity of the band corresponding to the 18S-RNA (left profile, in image below). Small and diffuse bands representing low molecular weight RNAs (tRNA and 5S ribosomal RNA) may be present. Degradation of the RNA is apparent as a smearing of ribosomal RNA bands and high molecular weight background as shown below (right profile, in image below).

Reverse transcription is performed with the high-capacity RNA-to-cDNA kit (Applied Biosystems; 438706) from total RNA according to the manufacturer's instructions. The cDNAs are then stored at −20° C. until use in polymerase chain reactions.

The microfluidic TaqMan qPCR arrays (or TaqMan low density arrays) were designed by StratiCELL and manufactured on demand by Applied Biosystems. Among the represented genes, 3 internal controls or reference genes, and 93 genes of interest are studied.

The TaqMan arrays are processed as described by the manufacturer's instructions (Micro Fluidic Card Getting Started Guide, Applied Biosystems). Briefly, cDNAs are mixed with a specific buffer (TaqMan Fast Advanced Master Mix, 4444557, Applied Biosystems) before being injected into the arrays and dispersed into the wells by centrifugation. Arrays are sealed and qPCRs are run using the Quantstudio7 Real-Time PCR System (Applied Biosystems) and its software (QuantStudio real time PCR Software v1.3. software, Applied Biosystems).

Threshold cycles (Ct) are obtained for each gene. Results files are exported from the qPCR device and analyzed using the DataAssist Software (v3.00, Applied Biosystems) designed to perform relative quantification of gene expression using the comparative Ct (ΔΔCt) method, through a combination of statistical analysis.

The data obtained for the conditions treated with the formulations is compared to the untreated condition or to the condition treated with the vehicle. The effects of the cytokine cocktail stimulation are analyzed as well by comparison of the data obtained for the untreated control (unstimulated) to the data obtained for the stimulated untreated control. The Ct values are normalized to the mean Ct of 3 housekeeping genes present on the array. The maximum Ct cut-off value was fixed at 36 cycles meaning that Ct values above 36 are not considered in the analysis.

The solubility of BETi was determined as described in Example 8 below.

### Example 4. In-Vitro Human Skin Model for Vitiligo

Part A Morphological Analysis after H/E Staining of Histologic Sections of Paraffin-Embedded Epidermis

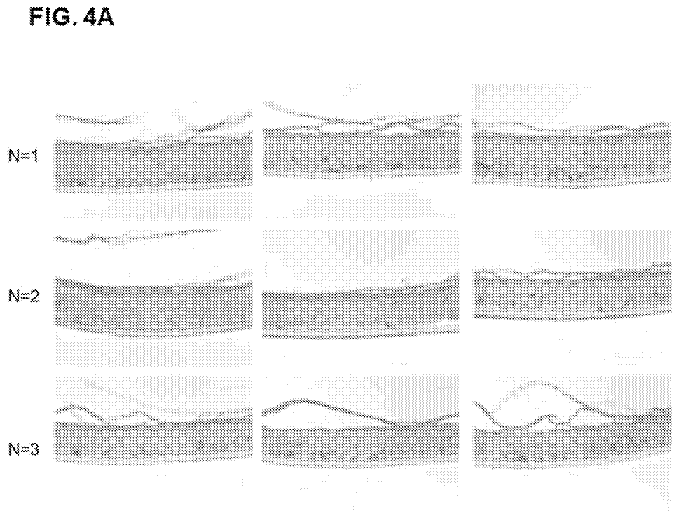

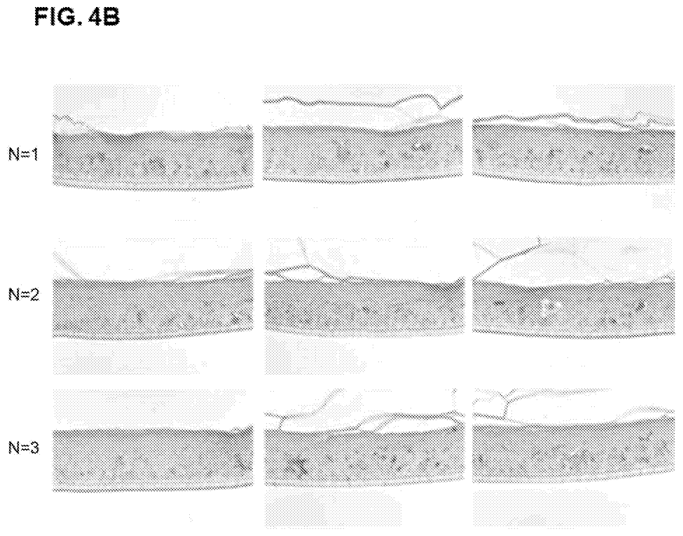

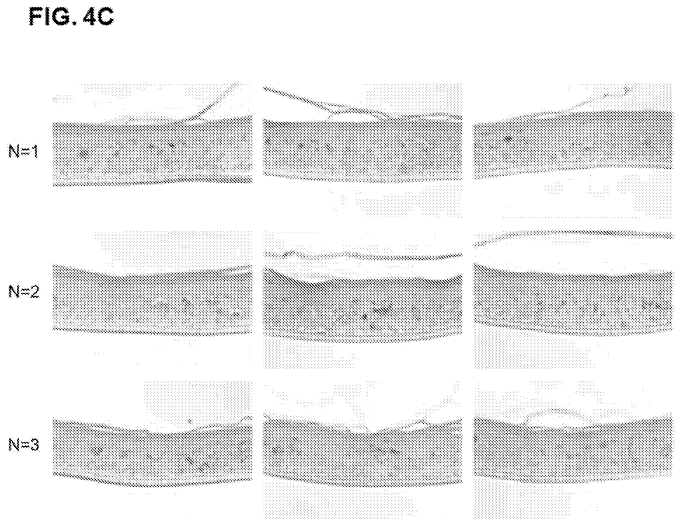

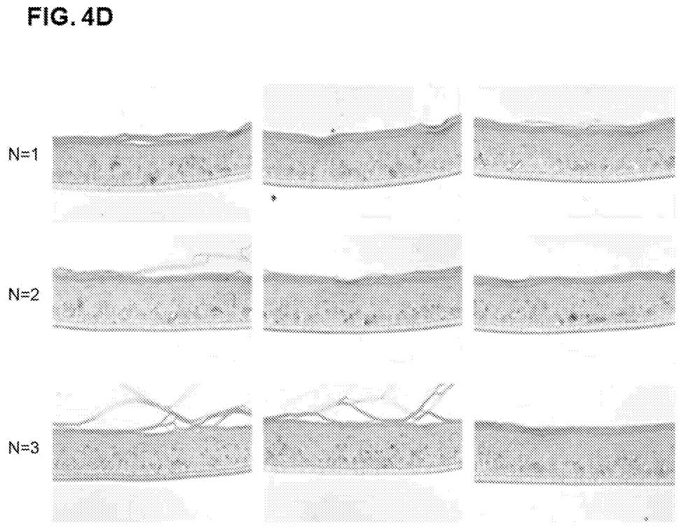

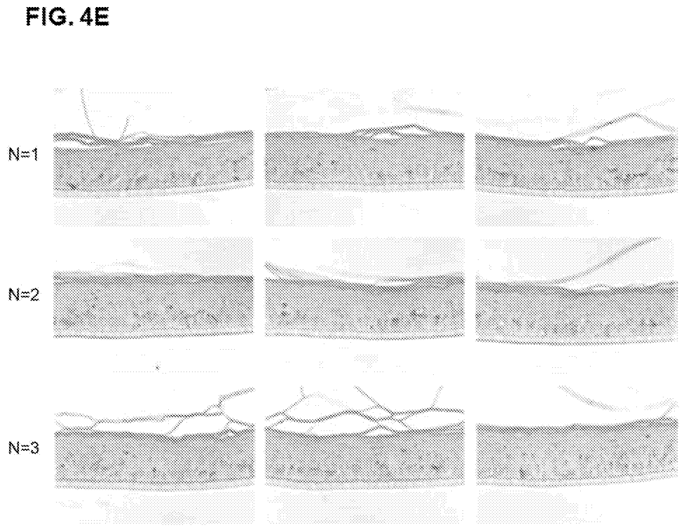

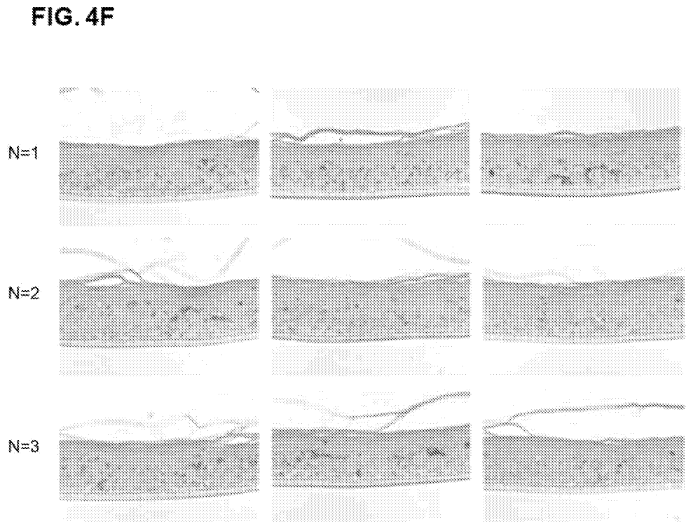

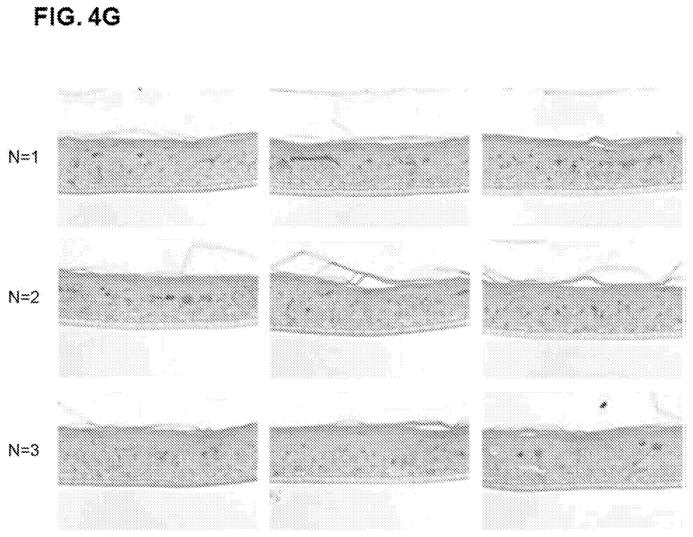

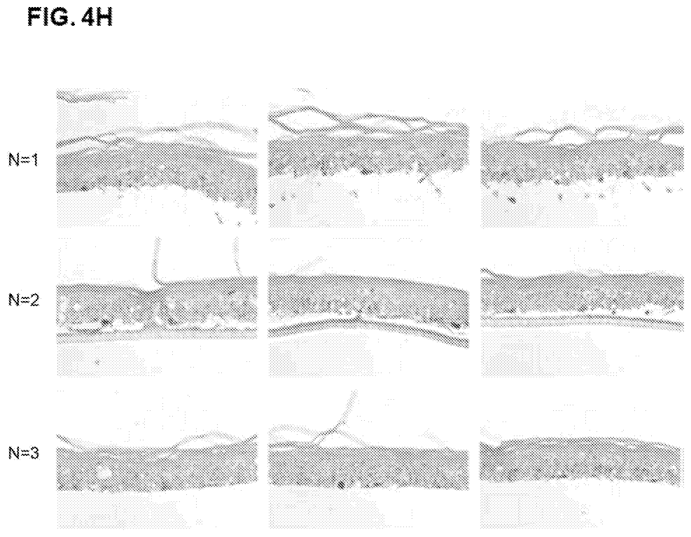

Formulations containing four doses of BETi1, the BETi1 vehicle (placebo) and JAK inhibitor, were evaluated in RHE TNF-α/IFN-γ vitiligo model as described in Experimental section Methods C and D and representative images of H/E stained epidermis sections are shown in FIG. 4A-H. Treatments of the RHEs with each of the formulations were initiated at day 9 of their reconstitution process. From this time, the culture medium was replaced by a culture medium depleted from hydrocortisone. Twenty-four hours later (at day 10) the RHE was stimulated with the cytokine cocktail (TNF-α and IFN- [symbol] ) to induce melanocytorhagy and was pursued for an additional 24 hours in the presence of the formulations (until day 11).

In order to confirm an absence of cytotoxic effect of the formulations and vehicle on the 3D model, a preliminary morphological analysis was conducted after hemalum/eosin staining of sections of paraffin-embedded tissues. Appropriate H/E staining with nuclei stained in blue and cytoplasm, in varying pink shades, and a multi-layered stratified human epidermis-like structure is observed. The epidermis presents a good alignment of the cells in the basal layer evidencing an oriented cell division and thus of a good structure). The melanin is well visible (in brown) and is, as expected, more abundant in the basal layers. It should be noted that the epidermal thickness and melanin abundance were increased at day 14 in comparison to day 9 of the reconstitution process, which is in correlation with the differentiation/maturation status of the epidermis. It was observed that the absence of hydrocortisone was responsible for a small decrease of epidermis thickness. When stimulated with the cytokine cocktail, the tissue thickness returned to its normal level (not shown).

BETi1 formulations at all four concentrations and corresponding vehicle did not induce any morphological alteration (FIGS. 4C-G), whereas the formulation containing Ruxolitinib at 1.5% was shown to be extremely deleterious to the epidermis, inducing a full loss the epidermal structure (FIG. 4H).

Melanin appears more abundant in the basal layers (bottom inner layer) of unstimulated control (FIG. 4A) compared to cytokine cocktail stimulated control (FIG. 4B). It is postulated that the cytokine cocktail induces the detachment of the melanocytes from the basal membrane (melanocytorhagy) but, given the fact that H/E staining is not capable of specifically highlighting the melanocytes (only melanin in a non-quantitative way), this was later confirmed using TRP-1 staining (see PART C). The highest abundance of melanin in the basal layer is observed in RHE treated with a formulation comprising 1% BETi1 (FIG. 4D) and a similar tendency (but to a lesser extent) is observed with RHE treated with 0.1% BETi1 formulation (FIG. 4E). In contrast, the lowest density of melanin in the basal layer is observed with vehicle (FIG. 4C) and stimulated untreated RHE (FIG. 4B).

### Part B: Melanin Content and Localization Analysis by Fontana Masson Staining of RHE-MEL Sections

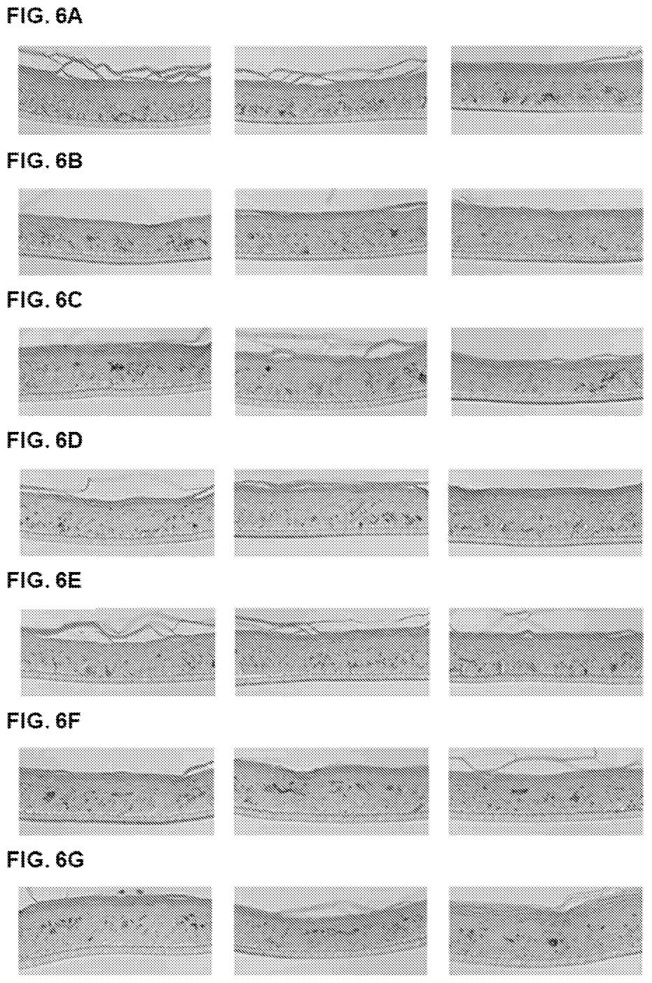

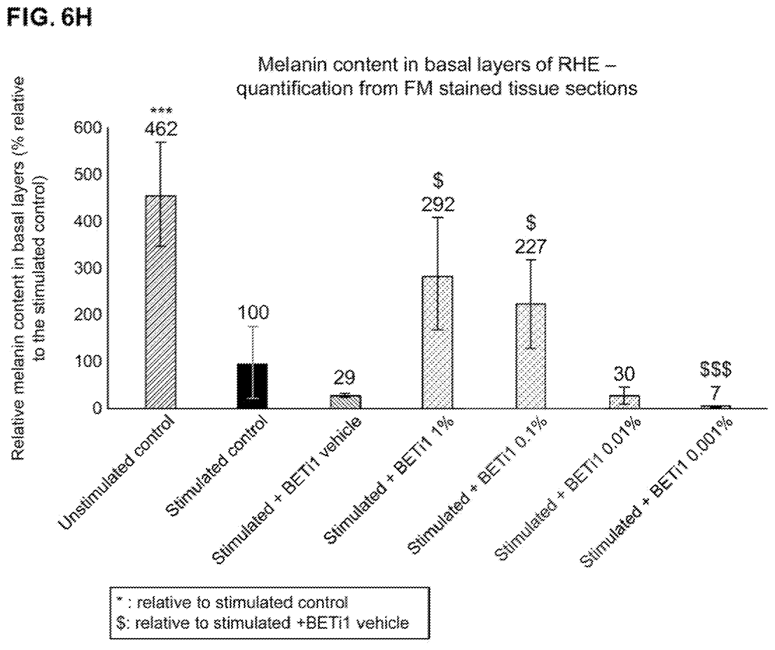

Formulations containing four doses of BETi1 the BETi1 vehicle (placebo), were evaluated in RHE TNF-α/IFN-γ vitiligo model as described Experimental section Method F and representative images of H/E stained epidermis sections are shown in FIG. 6A-G. The images according to this method are consistent with the images with H/E stained epidermis sections and shown in FIG. 4A-H.

Results of the quantification of melanin in basal layers of epidermis stimulated or not with the cytokine cocktail to induce melanocytorhagy and treated with the test items are shown in FIG. 6H. The results are expressed with normalization to the surface of region of interest (ROI) corresponding to the 2-3 basal layers of the RHEs. A statistical analysis (student t-test) was performed to compare the effects of the treatments with the formulations to the vehicle (with $: 0.05>p>0.01; $$: 0.01>p>0.001; and $$$: p<0.001). The same analysis was performed to compare the effects of the vehicle and of the unstimulated control to the stimulated control (with *: 0.05>p>0.01; **: 0.01>p>0.001; and ***: p<0.001). In accordance with PART A, less melanin was found in the basal layers of stimulated RHE in comparison with unstimulated RHE. The formulations containing BETi1 1% and BETi1 0.1% were able to significantly increase the melanin content in the basal layers which may indicate a reduction of the melanocytorhagy process. The vehicle tended to accentuate (in a unsignificant way) this loss of melanin in the basal layers. This tendency was observed as well for TRP-1 staining which was significantly less abundant in the basal layers after the treatment with the vehicle. As for TRP-1 staining, the effects of the ruxolitinib cream at 1% on melanin content could not be assessed given that the sections have detached from the glass slides during the staining process (probably without being bound by any theory may be due to the bad structure of the epidermis).

Part C: Melanocyte Localization after TRP1 Immunostaining

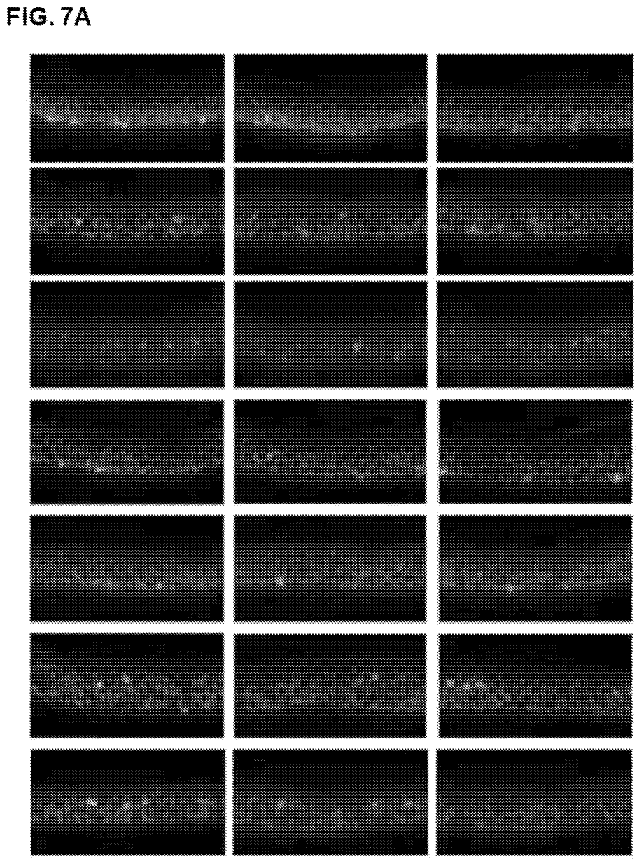

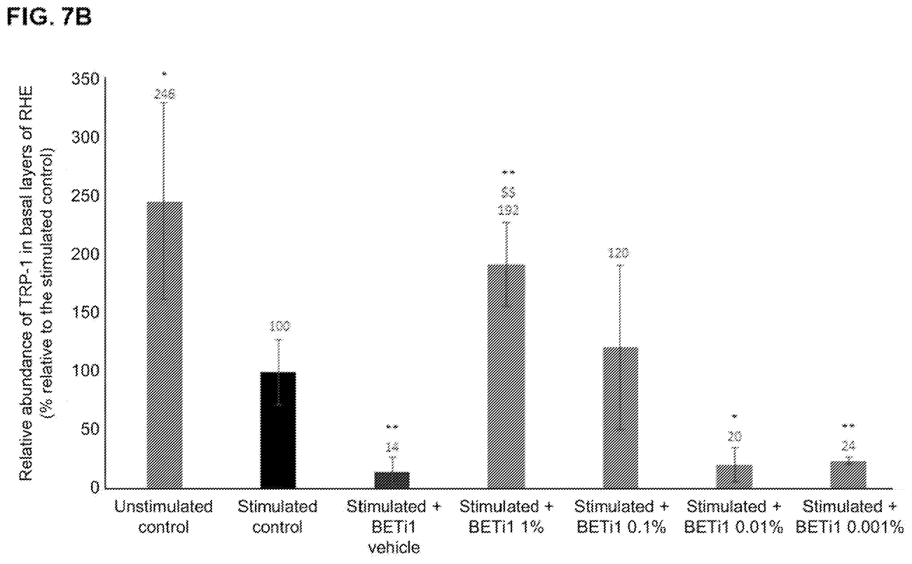

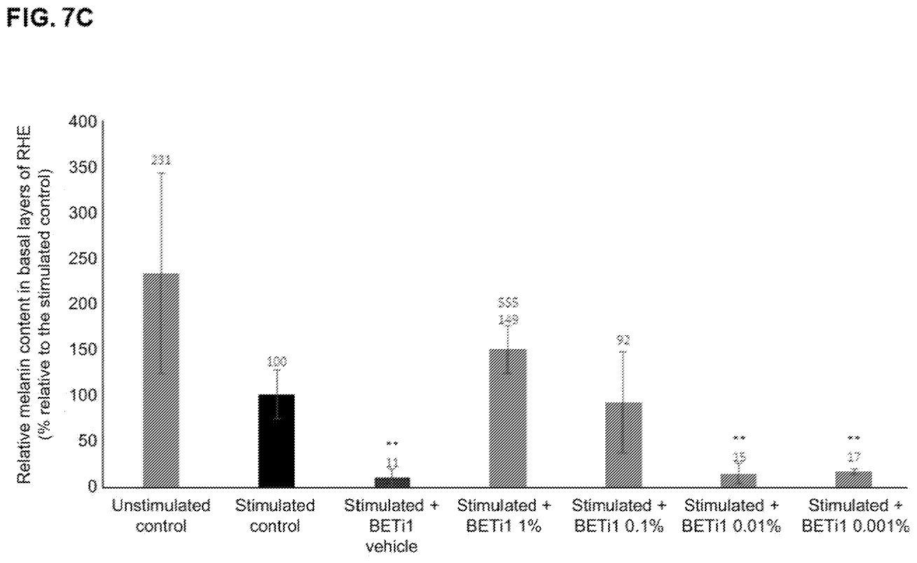

Formulations containing four doses of BETi1 the BETi1 vehicle (placebo), were evaluated in RHE TNF-α/IFN-γ vitiligo model as described in Experimental section Method G and representative images of RHE after TRP-1 staining are shown in FIG. 7A and respective quantification with and without normalization are shown in Table 19 and Table 20 and FIGS. 7B and FIG. 7C respectively. The stimulation with the cytokine cocktail (TNF-α and IFN- [symbol] ) in accordance with Part A and B, reduced the expression of TRP-1 and thus the abundance of melanocytes in the basal layers of the RHE, confirming the melanocytorhagy. In the model, stimulated treated with the vehicle induced a significant decrease of TRP-1 abundance as compared to the stimulated untreated control. Without being bound by any theory the barrier effect provided by petrolatum in the vehicle, which increases skin hydration, has some effect on retention of melanocytes in the basal layer. The images and their quantifications revealed that the BETi1 formulation at 1% is able to reverse/prevent the melanocytorhagy, as TRP-1 is significantly increased in the basal layers following the treatment. A dose response could clearly be observed with the BETi1 formulation, with a tendency to the increase for the dose of 0.1% and with the two last concentrations being not significant. The effects of the ruxolitinib cream at 1% could not be assessed given that the sections have detached from the glass slides during the immunostaining process (probably without being bound by any theory may be due to the bad structure of the epidermis).

Part D: Quantification of MMP-9 and E-Cadherin Secretion by ELISA from the Collected Culture Medium

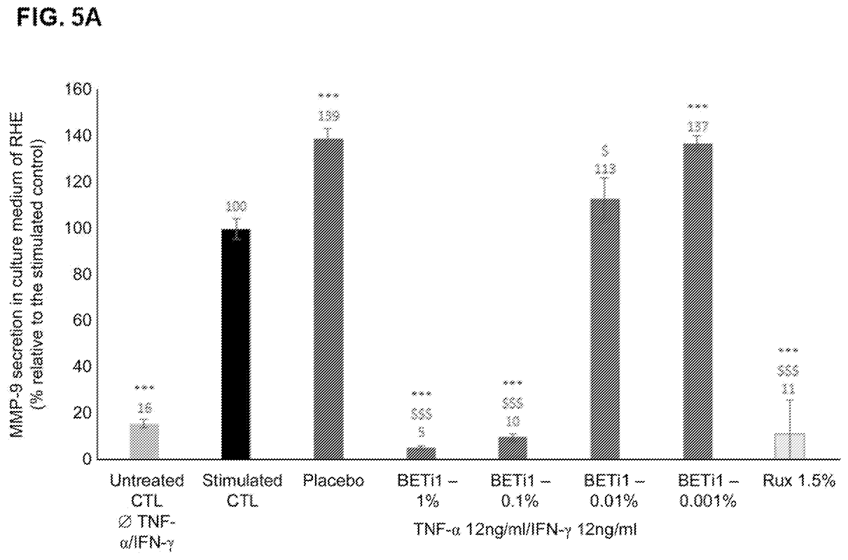

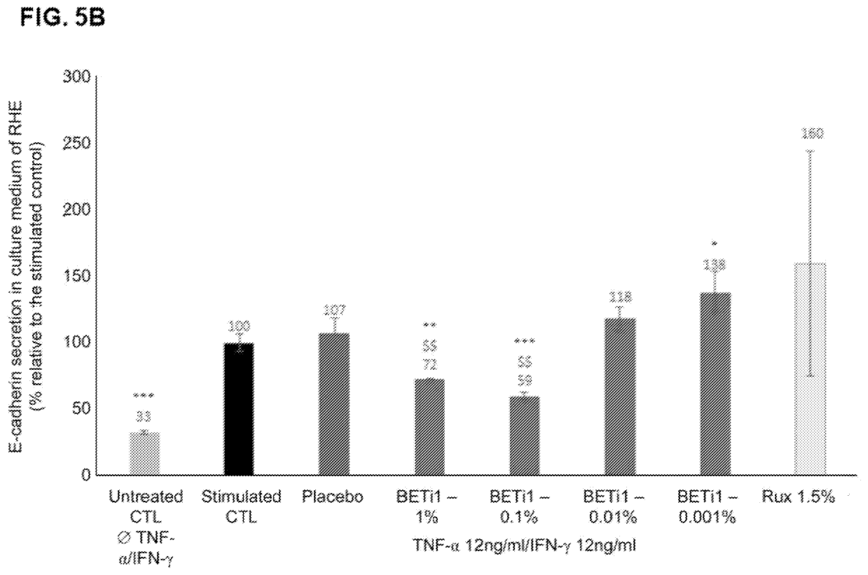

Quantification of secretion of MMP-9 and E-cadherin by ELISA from collected culture medium was conducted in accordance with Method E. The results, shown in FIG. 5A and FIG. 5B are based on Tables 23 and 24 respectively and presented in percentage of release in comparison to the stimulated control. A statistical analysis (student t-test) was performed to compare the effects of the treatments with the formulations to the vehicle (with $: 0.05>p>0.01; $$: 0.01>p>0.001; and $$$: p<0.001). The same analysis was performed to compare the effects of the vehicle and of the unstimulated control to the stimulated control (with *: 0.05>p>0.01; **: 0.01>p>0.001; and ***: p<0.001). The analysis was performed from three replicates per condition (n=3). The mean of the three values and the standard deviation are presented on the histograms in FIG. 5A and FIG. 5B. As shown in Table 21 and FIG. 5A and consistent with the results of Part A, MMP-9 secretion was increased following the stimulation with the cytokines, which validates the assay. Surprisingly, TNF-α/IFN-γ stimulation of RHE and treated with vehicle generated an increase in MMP-9 secretion (expressed as percent of untreated stimulated control) in subjects which was statistically significant compared to stimulated control.

The two highest concentrations of BETi1 formulation (1% and 0.1%) induced a highly significant decrease of MMP-9 secretion, with a return to the basal production level. The greatest inhibition of MMP-9 secretion (to about 5%) was observed with BETi1 10% which was more than that seen for 0.1% BETi1 (to about 10%). This inhibition was greater than that observed with the positive control JAK inhibitor (Ruxolitinib 1.5%) (to about 11%). As mentioned above Ruxolitinib at 1.5% was shown to be extremely deleterious to the epidermis, inducing a loss the epidermal structure. Without being bound to any theory, the observed reduction in MMP-9 secretion for cells treated with the positive control formulation (Ruxolitinib 1.5%) may without being bound by any theory be attributed to increased cell death and not necessarily to reduction of vitiligo symptoms. The decrease in the levels of MMP-9 observed for 0.1% BETi1, 1% BETi1 and positive control was statistically significant compared to placebo and stimulated untreated control. The decrease observed for 0.01% BETi1 was statistically significant compared to placebo. The decrease observed for 0.001% BETi1 was statistically significant compared to stimulated untreated control.

MMP9 plays several important functions within neutrophil action, such as degrading extracellular matrix, activation of IL-1β, cleavage of several chemokines and cleavage of E-cadherin. E-cadherin, as discussed further detail below, is a major protein involved in melanocyte adhesion to the basal layer. Interleukin-10 is expressed at high levels in people with vitiligo.

A dose dependent inhibitory effect of BETi1 on MMP-9 secretion is clearly shown in FIG. 5A, wherein the higher BETi1 dose there is stronger the inhibition of MMP-9 secretion. Without being bound by any theory, it is postulated that treatment with BETi1 results in reduction of MMP-9 secretion which consequentially reduces activation of IL-1β and may result in improve vitiligo symptoms.

Treatment with topical BETi1 may be capable of improved melanocyte adhesiveness to the basal membrane. As shown in Table 22 and FIG. 5B, stimulation with the cytokine cocktail (TNF-α/IFN-γ stimulation) increased secretion of soluble form of E-cadherin into the medium (expressed as percent of untreated stimulated control) compared to unstimulated control. Treatment with BETi1 formulations at 1 and 0.1% was able to significantly reduce secretion of E-cadherin. The greatest decrease of E-Cadherin secretion (59%) was observed with for cells treated with a formulation containing BETi1 0.1% followed by cells treated with 1% BETi1 (to about 118%). The decrease observed for 1% and 0.1% BETi1 was statistically significant compared to placebo and stimulated untreated control. The decrease observed for 0.001% BETi1 was statistically significant compared to stimulated untreated control. These results are in accordance with those observed for MMP-9 secretion which is at least partially responsible for the cleavage of E-cadherin into its soluble form (which is secreted).

Without being bound by any theory, it is postulated that E-cadherin, which mediates the adhesion between melanocytes and keratinocytes in the epidermis, is absent from or discontinuously distributed across melanocyte keratinocyte junction of vitiligo subjects. This absence may be due to high levels of MMP-9 which is at least partially responsible for the cleavage of E-cadherin into its soluble form that is secreted into the skin. Treatment with BETi1 formulations at 1% and 0.1% may result in reduction of MMP-9 secretion which in turn may result in less cleavage of E-cadherin and higher levels of E-cadherin being maintained at the cell junction between keratinocytes and melanocyte. Hence less detachment of melanocytes from the basal layer and thus improvement in vitiligo symptoms.

In other words, in vitiligo IFN-γ and TNF-α stimulate keratinocytes to produce MMP-9. Increased levels of MMP-9 result in E-cadherin cleavage and release of its soluble form and consequently melanocyte detachment. BETi1 inhibition of MMP-9 secretion indirectly prevents melanocyte detachment via retention of E-cadherin and also directly suppresses the IL-1β signaling pathway which may be responsible for the inflammatory response which triggers vitiligo. Therefore, treatment with BETi formulations may result in stabilization of melanocytes in the basal layer of the epidermis by preventing E-cadherin disruption which in turn may be useful to prevent depigmentation occurring in vitiligo and during chronic skin inflammation.

### Part E: Gene Expression Analysis (TaqMan Low Density Array 96 Genes)

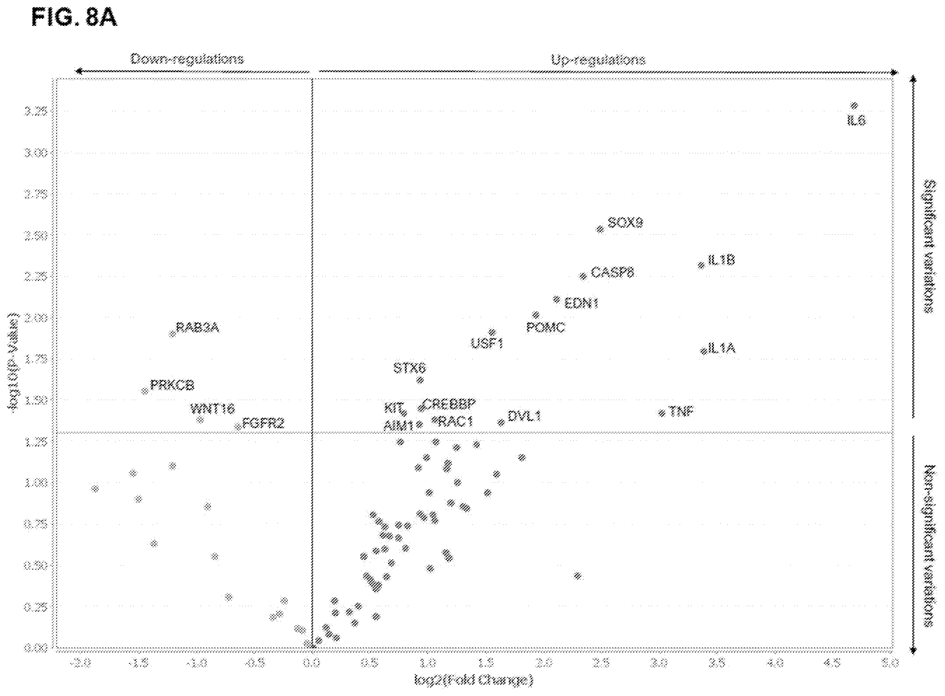

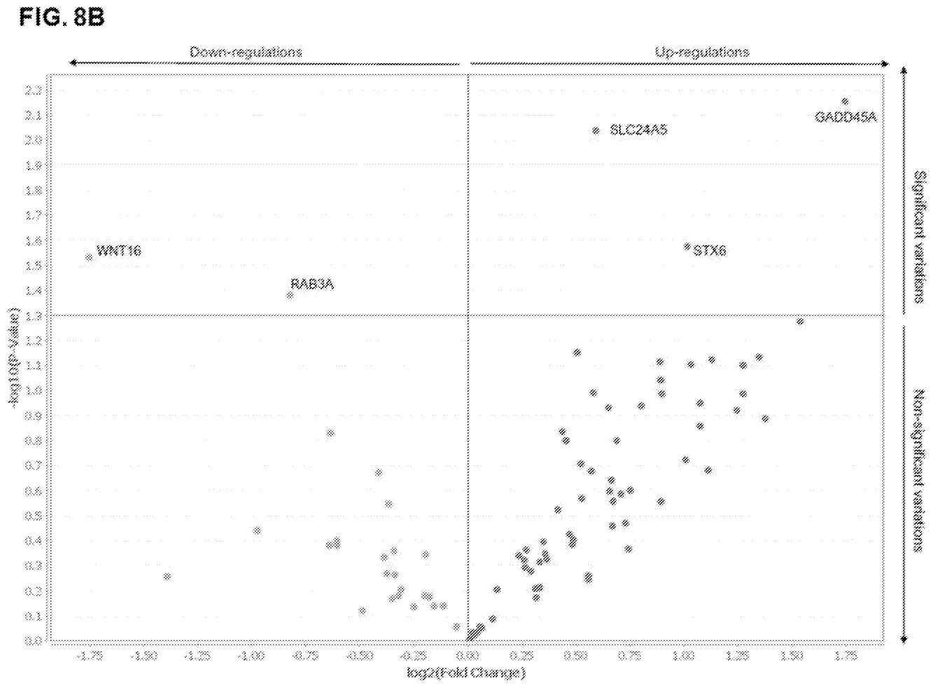

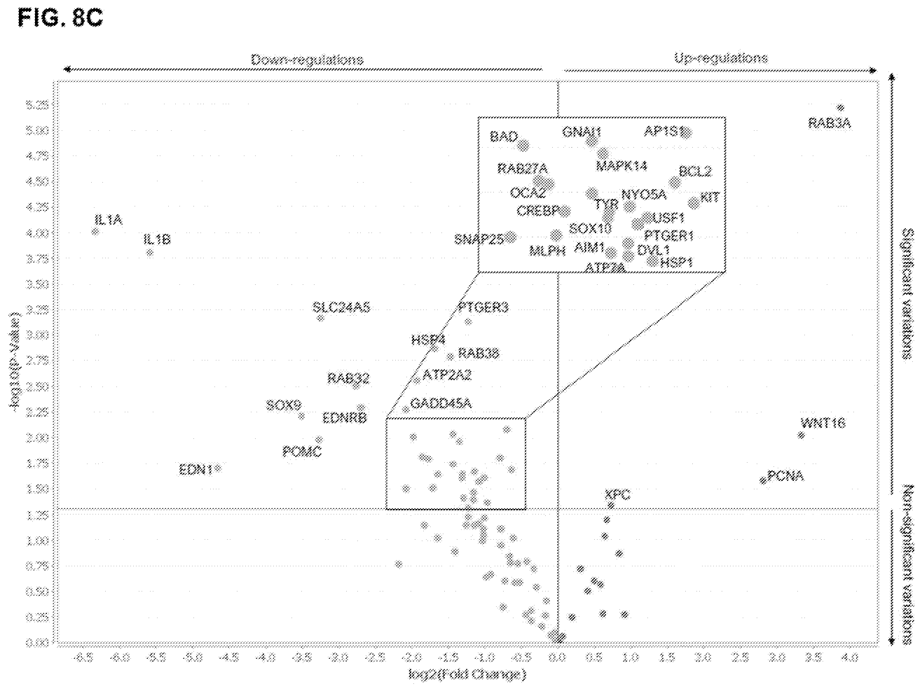

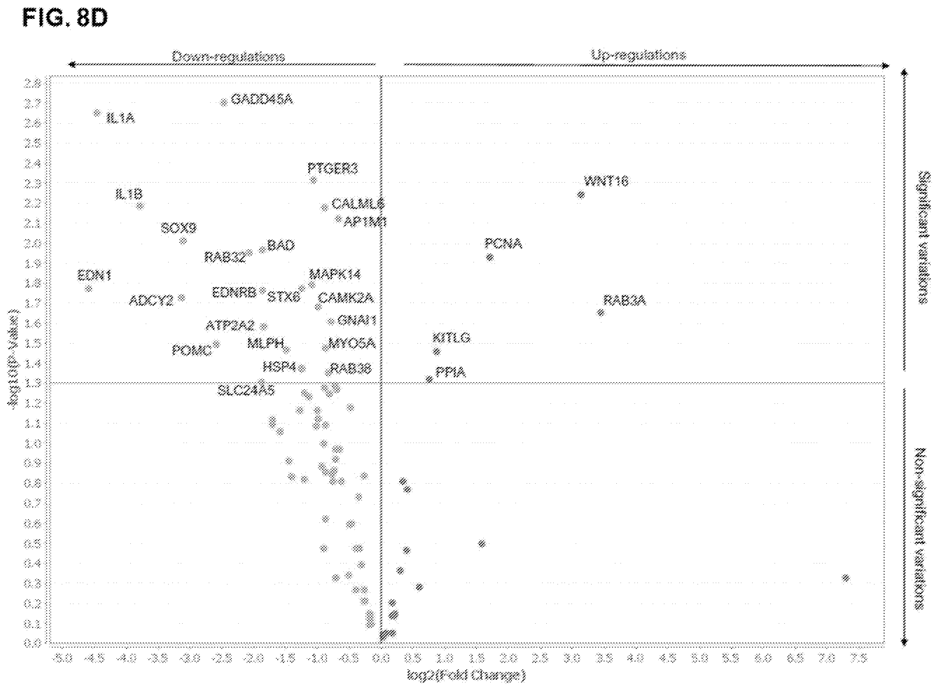

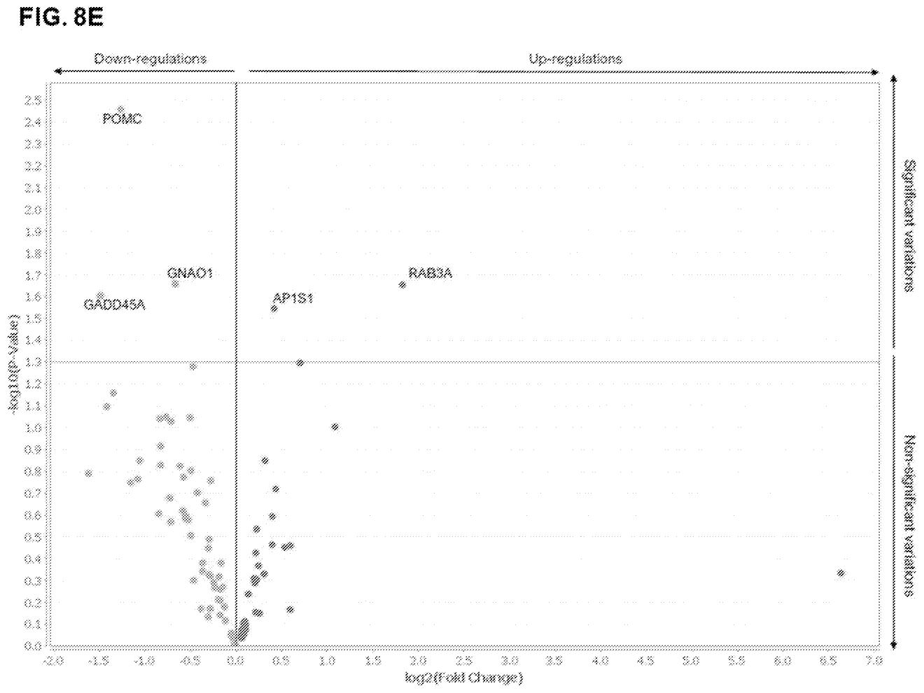

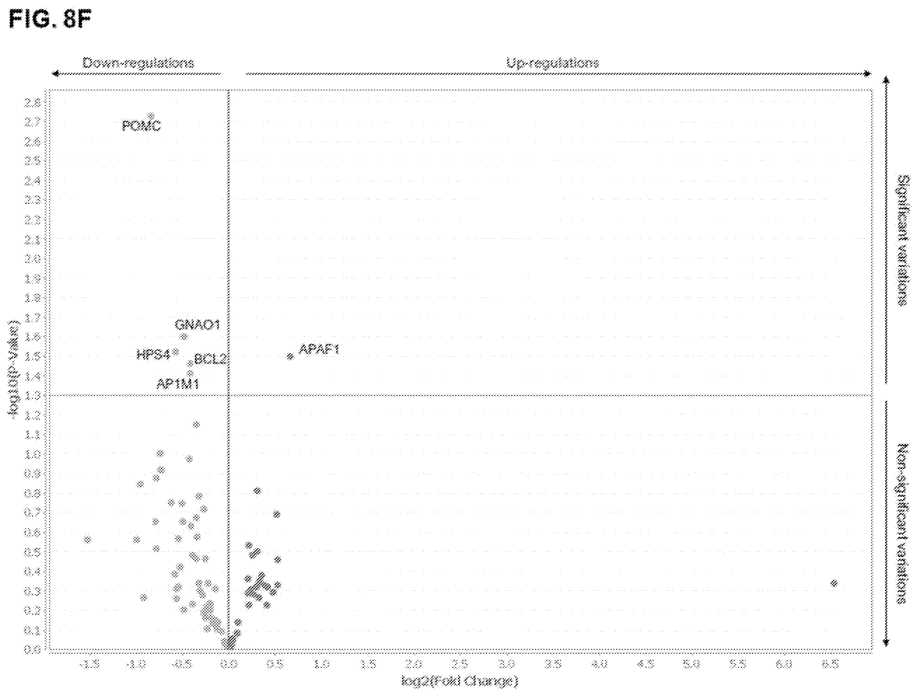

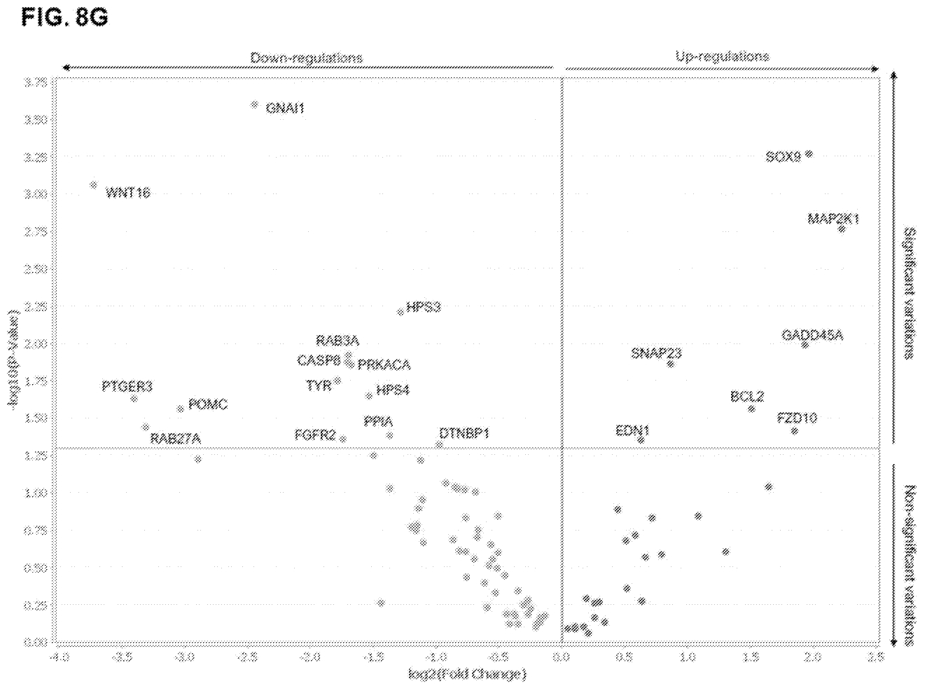

Data relative to gene expression analysis of 92 target genes related to melanogenesis (including melanogenic factors and other factors from melanogenic pathways, markers involved in melanosome biogenesis, maturation and transport and apoptosis/DNA damages related markers, etc.) from RHE stimulated with the cytokine cocktail (TNF-α/IFN-γ) and treated with formulations containing four doses of BETi1 were compared to those treated with BETi1 vehicle, JAK inhibitor, and untreated as described Experimental section Method H. The fold change or relative quantifications (RQ) of the genes expression and statistical significance of such change between (i) stimulated versus unstimulated, (ii) vehicle versus untreated, (iii) each of BETi1 at four dose (1%, 0.1%, 0.01%, 0.001%) vs vehicle, (iv) JAK inhibitor versus untreated stimulated control are shown in Tables 23-29 and the transcriptome volcano plots of genes relevant to vitiligo are shown in FIGS. 8A-G. The mean of three housekeeping genes (18S, POLR2A and RPLP0) was used for data normalization. Housekeeping genes are cellular maintenance genes which regulate basic and ubiquitous cellular functions. In many RT-qPCR reactions, these genes are used as internal control genes. Housekeeping serve as control for expression of genes overexpressed in vitiligo and downregulated by BETi1. Volcano plot was used to highlight significant variations of gene expression. This figure is constructed by plotting the negative logarithm of the p-value on the y-axis, and the logarithm with base 2 of the fold change (log 2RQ) between the test conditions on the x-axis. This results in datapoints with significant p-values appearing at the top of the plot, above the horizontal threshold line fixed at a p-value of 0.05. Genes with fold increase (red dots) or decrease (green dots) lie on both sides of the vertical threshold line. Gene symbols are given for significant expression changes.

The following genes IL6, IL1A, IL1B, TNF, SOX9, CASP8, EDN1, POMC, DVL1, USF1, RAC₁, CREBBP, STX6, AIM1, KIT, were over expressed in Stimulated RHE in descending order, e.g., IL6 the gene with highest overexpression in stimulated RHE. FGFR2, WNT16, RAB3A, and PRKCB were downregulated. As expected, IL1A, IL1B, and TNF involved in inflammation were overexpressed (Table 23 and FIG. 8A). The vehicle caused GADD45A, SLC₂₄A5, and STX6 to be overexpressed compared to stimulated untreated. Interestingly the vehicle has some effect on downregulation of RAB3A and WNT16 (Table 24 and FIG. 8B).

Treatment with BETi1 1% caused RAB3A (14.5685) WNT16 (10.0801) PCNA (7.0134), and XPC (1.6569) to be overexpressed compared to vehicle. In contrast, treatment with BETi1 1% caused many genes to be downregulated with lowest expression observed for IL1B (0.0208), IL1A (0.0124), SOX9 (0.0882), and EDN1 (0.0395) which indicate that BETi treats vitiligo also by reducing cytokine involved in inflammation (Table 25 and FIG. 8C). Treatment with BETi1 0.1% caused RAB3A (10.8195), WNT16 (8.7922), and PCNA (3.2615), to be overexpressed compared to vehicle but to a lesser extent seen for BETi1 1%. Surprisingly, KITLG (1.8415) and PPIA (1.6969) were overexpressed compared to vehicle but this was not the case for 1% BETi. Similarly, treatment with BETi1 0.1% caused inter alia EDN1 (0.0413), IL1B (0.0727), IL1A (0.0454), SOX9 (0.1155) to be downregulated compared to vehicle but to a lesser extent seen for BETi1 1%. (Table 26 and FIG. 8D). This clearly shows a dose effect of BETi1 on upregulation and/or downregulation of genes involved in the inflammatory response as well as detachment of melanocytes from the basal layer.

Surprisingly, treatment with BETi1 at 0.01% had a different effect on gene expression pattern than at 0.1% or 1%. The only genes which were upregulated albeit to a much lesser extent was RAB3A (3.5532) and AP1S1 (1.3312) (Table 27 and FIG. 8E). The gene expression pattern of 0.01% was also very different than with BETi at 0.001%. For BETi1 0.001%, a gene associated with cell death was upregulated APAF1 (1.5797) whereas a few genes which were downregulated with vehicle were also downregulated for BETi1 0.001% e.g., HSP4 (0.6711), and POMC (0.5586). Regardless of their identity, the number of genes that was upregulated or downregulated was greater for the BETi1 at higher doses (1% and 0.1%) compared to BETi1 at lower doses (0.01% and 0.001%) and vehicle (Table 28 and FIG. 8F).

Treatment with JAK inhibitor (ruxolitinib cream at 1.5%) induced overexpression of MAP2K1 (4.6701) and BCL2 (2.8444) associated cell death pathways which corresponds with the morphology of skin section A. RAB3A (0.3077) and WNT16 (0.0757) were downregulated compared to stimulated untreated, whereas and in BETi1 1% treatment they were upregulated (Table 29 and FIG. 8G).

Applicants have discovered the previously unrecognized role of BETi1 at 1% and 0.1% in WNT pathway activation. This activation may result in melanocyte differentiation and proliferation of new melanocytes and hence re-pigmentation of vitiligo skin. These results support further clinical exploration of BETi1 as WNT agonists to repigment vitiligo subjects.

TABLE 18
Quantification of TRP-1 in basal layers of epidermis Results expressed without normalization
to the surface of region of interest (ROI) corresponding to the basal layers of the RHEs

| | | | | | | | | | | | | | Student |
|---|---|---|---|---|---|---|---|---|---|---|---|---|---|
| | | | | | | Marked | | | | | | Student- | t- test |
| | | | | | | area* | | % staining | | | | t test | (p value |
| | | | Mean | Total | Marked | Inten- | Mean | relative | Mean | Mean | | (p value | vs stimu- |
| | | Marked | staining | Area | area * | sity/ | (stimu- | to stimu- | per | per | | vs stimu- | lated + |
| | | area | inten- | ROI | Inten- | Total | lated | lated | RHE | condition | | lated | BETi1 |
| | | [μm²] | sity | [μm²] | sity | area ROI | control) | control | (%) | (%) | SD | control) | vehicle |
| Unstimulated | RHE1 | 70 | 132916 | 4217 | 9354495 | 2219 | | 397 | 377 | 462 | 104 | 0 | 0 |
| control | | 90 | 156196 | 5057 | 13997192 | 2768 | | 496 | | | | | |
| | | 56 | 105175 | 4408 | 5888433 | 1336 | | 239 | | | | | |
| | RHE2 | 74 | 127787 | 4543 | 9509142 | 2093 | | 375 | 430 | | | | |
| | | 79 | 136438 | 4376 | 10744765 | 2455 | | 440 | | | | | |
| | | 78 | 136848 | 4016 | 10634458 | 2648 | | 474 | | | | | |
| | RHE3 | 88 | 148471 | 4259 | 13105238 | 3077 | | 551 | 578 | | | | |
| | | 48 | 86097 | 4454 | 4116814 | 924 | | 166 | | | | | |
| | | 119 | 206129 | 4321 | 24529969 | 5677 | | 1017 | | | | | |
| Stimulated | RHE1 | 16 | 29790 | 3611 | 487841 | 135 | 558 | 24 | 94 | 100 | 77 | 1 | 0 |
| control | | 33 | 54460 | 3873 | 1805621 | 466 | | 83 | | | | | |
| | | 51 | 87678 | 4586 | 4437121 | 968 | | 173 | | | | | |
| | RHE2 | 18 | 33141 | 4249 | 585071 | 138 | | 25 | 180 | | | | |
| | | 36 | 67924 | 4100 | 2423325 | 591 | | 106 | | | | | |
| | | 70 | 125187 | 3835 | 8781117 | 2290 | | 410 | | | | | |
| | RHE3 | 22 | 40804 | 4407 | 898708 | 204 | | 37 | 26 | | | | |
| | | 12 | 21693 | 4538 | 254567 | 56 | | 10 | | | | | |
| | | 20 | 38513 | 4412 | 787398 | 178 | | 32 | | | | | |
| Stimulated + | RHE1 | 24 | 38212 | 5138 | 901994 | 176 | | 31 | 34 | 29 | 4 | 0 | 1 |
| BETi1 | | 32 | 51221 | 4734 | 1620735 | 342 | | 61 | | | | | |
| vehicle | | 11 | 19463 | 4605 | 212692 | 46 | | 8 | | | | | |
| | RHE2 | 27 | 51822 | 5294 | 1420182 | 268 | | 48 | 28 | | | | |
| | | 21 | 38468 | 5715 | 804558 | 141 | | 25 | | | | | |
| | | 13 | 22832 | 5288 | 288665 | 55 | | 10 | | | | | |
| | RHE3 | 7 | 14094 | 4128 | 104267 | 25 | | 5 | 26 | | | | |
| | | 15 | 28455 | 4535 | 425801 | 94 | | 17 | | | | | |
| | | 27 | 48275 | 4220 | 1306756 | 310 | | 55 | | | | | |
| Stimulated + | RHE1 | 51 | 90823 | 4198 | 4629884 | 1103 | | 197 | 233 | 292 | 121 | 0 | 0 |
| BETi1 1% | | 85 | 137848 | 4649 | 11778285 | 2533 | | 454 | | | | | |
| | | 26 | 47496 | 4677 | 1248955 | 267 | | 48 | | | | | |
| | RHE2 | 118 | 206316 | 5630 | 24378711 | 4330 | | 775 | 431 | | | | |
| | | 78 | 133509 | 5165 | 10379390 | 2010 | | 360 | | | | | |
| | | 52 | 87760 | 5129 | 4526836 | 883 | | 158 | | | | | |
| | RHE3 | 61 | 116435 | 5230 | 7102302 | 1358 | | 243 | 210 | | | | |
| | | 45 | 80678 | 4484 | 3662378 | 817 | | 146 | | | | | |
| | | 62 | 119810 | 5541 | 7485369 | 1351 | | 242 | | | | | |
| Stimulated + | RHE1 | 47 | 93707 | 5314 | 4420815 | 832 | | 149 | 123 | 227 | 96 | 0 | 0 |
| BETi1 0.1% | | 33 | 54495 | 4451 | 1782967 | 401 | | 72 | | | | | |
| | | 49 | 84404 | 4949 | 4095451 | 828 | | 148 | | | | | |
| | RHE2 | 47 | 85955 | 4809 | 4026218 | 837 | | 150 | 247 | | | | |
| | | 80 | 135464 | 5759 | 10895911 | 1892 | | 339 | | | | | |
| | | 61 | 114089 | 4946 | 6951443 | 1405 | | 252 | | | | | |
| | RHE3 | 49 | 95523 | 5169 | 4670311 | 904 | | 162 | 312 | | | | |
| | | 79 | 134000 | 5015 | 10615882 | 2117 | | 379 | | | | | |
| | | 80 | 147140 | 5346 | 11835059 | 2214 | | 396 | | | | | |
| Stimulated + | RHE1 | 24 | 44268 | 4679 | 1077704 | 230 | | 41 | 42 | 30 | 17 | 0 | 1 |
| BETi1 0.01% | | 12 | 22228 | 4483 | 263846 | 59 | | 11 | | | | | |
| | | 33 | 58455 | 4797 | 1949883 | 406 | | 73 | | | | | |
| | RHE2 | 6 | 12071 | 4784 | 72245 | 15 | | 3 | 11 | | | | |
| | | 16 | 31040 | 5143 | 511446 | 99 | | 18 | | | | | |
| | | 13 | 23491 | 4723 | 316753 | 67 | | 12 | | | | | |
| | RHE3 | 28 | 45338 | 4793 | 1266880 | 264 | | 47 | 39 | | | | |
| | | 31 | 56437 | 4778 | 1738316 | 364 | | 65 | | | | | |
| | | 8 | 12345 | 5382 | 95057 | 18 | | 3 | | | | | |
| Stimulated + | RHE1 | 13 | 22089 | 5007 | 284484 | 57 | | 10 | 6 | 7 | 1 | 0 | 0 |
| BETi1 0.001% | | 5 | 9372 | 4204 | 45698 | 11 | | 2 | | | | | |
| | | 8 | 16213 | 4167 | 135751 | 33 | | 6 | | | | | |
| | RHE2 | 5 | 9573 | 4908 | 46352 | 9 | | 2 | 8 | | | | |
| | | 13 | 25186 | 5111 | 317595 | 62 | | 11 | | | | | |
| | | 13 | 27072 | 4850 | 343192 | 71 | | 13 | | | | | |
| | RHE3 | 10 | 20448 | 5119 | 210410 | 41 | | 7 | | | | | |
| | | 12 | 25296 | 5501 | 307928 | 56 | | 10 | 6 | | | | |
| | | 5 | 10394 | 5709 | 53477 | 9 | | 2 | | | | | |

TABLE 19
Quantification of TRP-1 in basal layers of epidermis Results expressed without normalization
to the surface of region of interest (ROI) corresponding to the basal layers of the RHEs

| | | | | | | | % | | | | | |
|---|---|---|---|---|---|---|---|---|---|---|---|---|
| | | | | | | | staining | | | | Student- | Student |
| | | | | Total | | | relative | Mean | Mean | | t test (p | t-test (p |
| | | Mean | Marked | Area | Marked | Mean | to | per | per | | value vs | value vs |
| | | staining | area | ROI | area * | (stimulated | stimulated | RHE | condition | | stimulated | stimulated + |
| | | intensity | [μm²] | [μm²] | Intensity | control) | control | (%) | (%) | SD | control) | vehicle |
| Unstimulated | RHE1 | 64.2 | 1668.8 | 7966.7 | 107083.2 | | 386.3 | 343.2 | 246 | 84.1 | 0.046 | 0.009 |
| control | | 62.2 | 1192.5 | 7818.0 | 74208.1 | | 267.7 | | | | | |
| | | 62.8 | 1659.0 | 8132.3 | 104116.1 | | 375.6 | | | | | |
| | RHE2 | 55.2 | 1174.2 | 9707.0 | 64836.3 | | 233.9 | 199.1 | | | | |
| | | 50.7 | 854.5 | 11189.0 | 43348.2 | | 156.4 | | | | | |
| | | 52.8 | 1087.2 | 8855.9 | 57352.2 | | 206.9 | | | | | |
| | RHE3 | 54.6 | 706.1 | 10069.7 | 38584.4 | | 139.2 | 195.9 | | | | |
| | | 65.0 | 968.7 | 10483.8 | 62991.9 | | 227.2 | | | | | |
| | | 57.9 | 1059.6 | 8774.5 | 61330.3 | | 221.2 | | | | | |
| Stimulated | RHE1 | 40.6 | 188.1 | 9637.7 | 7629.1 | 27720.4 | 27.5 | 109.8 | 100 | 27.9 | 1.000 | 0.008 |
| control | | 53.2 | 779.5 | 6981.5 | 41474.9 | | 149.6 | | | | | |
| | | 59.1 | 713.8 | 9100.3 | 42194.4 | | 152.2 | | | | | |
| | RHE2 | 50.1 | 337.5 | 8688.4 | 16912.4 | | 61.0 | 121.7 | | | | |
| | | 61.2 | 762.4 | 8899.1 | 46690.6 | | 168.4 | | | | | |
| | | 55.4 | 679.0 | 8217.8 | 37638.1 | | 135.8 | | | | | |
| | RHE3 | 46.7 | 535.4 | 8064.9 | 24984.7 | | 90.1 | 68.5 | | | | |
| | | 62.6 | 228.5 | 8134.6 | 14313.3 | | 51.6 | | | | | |
| | | 52.1 | 338.6 | 8319.0 | 17645.9 | | 63.7 | | | | | |
| Stimulated + | RHE1 | 28.9 | 2.6 | 9909.9 | 74.7 | | 0.3 | 0.7 | 14 | 12.1 | 0.008 | 1.000 |
| vehicle | | 0.0 | 0.0 | 9585.5 | 0.0 | | 0.0 | | | | | |
| | | 41.0 | 11.5 | 10366.6 | 469.8 | | 1.7 | | | | | |
| | RHE2 | 41.5 | 226.7 | 10207.6 | 9417.4 | | 34.0 | 23.4 | | | | |
| | | 40.7 | 232.6 | 11838.5 | 9469.9 | | 34.2 | | | | | |
| | | 29.1 | 18.9 | 12804.8 | 550.8 | | 2.0 | | | | | |
| | RHE3 | 0.0 | 0.0 | 9824.1 | 0.0 | | 0.0 | 19.2 | | | | |
| | | 27.4 | 0.3 | 11632.9 | 9.2 | | 0.0 | | | | | |
| | | 44.3 | 359.5 | 10272.7 | 15925.7 | | 57.5 | | | | | |
| Stimulated + | RHE1 | 62.9 | 765.2 | 11042.5 | 48120.8 | | 173.6 | 220.8 | 192 | 36.3 | 0.026 | 0.001 |
| BETi1 1% | | 61.4 | 1106.0 | 10274.7 | 67964.3 | | 245.2 | | | | | |
| | | 65.1 | 1037.5 | 11327.5 | 67555.6 | | 243.7 | | | | | |
| | RHE2 | 43.8 | 408.3 | 9926.1 | 17884.2 | | 64.5 | 151.1 | | | | |
| | | 59.5 | 1056.5 | 10609.8 | 62851.0 | | 226.7 | | | | | |
| | | 47.2 | 951.2 | 10334.5 | 44942.6 | | 162.1 | | | | | |
| | RHE3 | 63.8 | 830.9 | 11896.8 | 53000.1 | | 191.2 | 203.4 | | | | |
| | | 57.8 | 1111.0 | 10214.3 | 64259.4 | | 231.8 | | | | | |
| | | 60.5 | 858.5 | 10159.6 | 51930.2 | | 187.3 | | | | | |
| Stimulated + | RHE1 | 51.3 | 855.0 | 11011.1 | 43824.4 | | 158.1 | 201.9 | 120 | 70.6 | 0.666 | 0.062 |
| BETi1 0.1% | | 57.2 | 803.1 | 11199.2 | 45938.4 | | 165.7 | | | | | |
| | | 60.9 | 1284.1 | 10557.5 | 78146.4 | | 281.9 | | | | | |
| | RHE2 | 60.5 | 449.4 | 10737.1 | 27165.0 | | 98.0 | 81.4 | | | | |
| | | 44.0 | 777.1 | 10052.9 | 34208.6 | | 123.4 | | | | | |
| | | 40.0 | 158.5 | 12283.3 | 6345.8 | | 22.9 | | | | | |
| | RHE3 | 55.1 | 363.9 | 11023.3 | 20044.0 | | 72.3 | 77.9 | | | | |
| | | 56.9 | 695.3 | 11573.6 | 39593.8 | | 142.8 | | | | | |
| | | 36.6 | 140.3 | 11200.6 | 5130.2 | | 18.5 | | | | | |
| Stimulated + | RHE1 | 50.7 | 236.2 | 12329.9 | 11983.0 | | 43.2 | 29.3 | 20 | 14.5 | 0.012 | 0.613 |
| BETi1 0.01% | | 61.1 | 198.9 | 10418.3 | 12155.6 | | 43.9 | | | | | |
| | | 32.8 | 6.9 | 10745.5 | 227.4 | | 0.8 | | | | | |
| | RHE2 | 27.5 | 0.1 | 10810.4 | 1.8 | | 0.0 | 28.1 | | | | |
| | | 60.9 | 310.6 | 10475.9 | 18905.6 | | 68.2 | | | | | |
| | | 43.1 | 104.1 | 11909.8 | 4484.0 | | 16.2 | | | | | |
| | RHE3 | 39.7 | 73.9 | 11528.2 | 2936.0 | | 10.6 | 3.7 | | | | |
| | | 29.8 | 1.8 | 10043.2 | 55.0 | | 0.2 | | | | | |
| | | 29.8 | 1.8 | 10043.2 | 55.0 | | 0.2 | | | | | |
| Stimulated + | RHE1 | 44.9 | 220.6 | 9321.0 | 9896.4 | | 35.7 | 21.6 | 24 | 3.0 | 0.009 | 0.270 |
| BETi1 0.001% | | 54.9 | 145.6 | 10966.5 | 7997.9 | | 28.9 | | | | | |
| | | 30.6 | 3.1 | 9257.7 | 93.5 | | 0.3 | | | | | |
| | RHE2 | 28.6 | 3.1 | 10655.4 | 88.3 | | 0.3 | 27.0 | | | | |
| | | 77.7 | 187.8 | 11411.9 | 14590.7 | | 52.6 | | | | | |
| | | 50.8 | 153.2 | 12080.8 | 7778.3 | | 28.1 | | | | | |
| | RHE3 | 50.2 | 132.7 | 12409.9 | 6660.6 | | 24.0 | 22.1 | | | | |
| | | 39.7 | 61.0 | 10824.0 | 2422.8 | | 8.7 | | | | | |
| | | 37.3 | 249.6 | 13752.4 | 9303.8 | | 33.6 | | | | | |

TABLE 20
Quantification of TRP-1 in basal layers of epidermis Results expressed with normalization
to the surface of region of interest (ROI) corresponding to the basal layers of the RHEs

| | | | | | | Marked | | % | | | | Student -t | Student |
|---|---|---|---|---|---|---|---|---|---|---|---|---|---|
| | | | | | | area* | | staining | | | | test (p | t-test |
| | | Mean | | Total | Marked | Inten- | Mean | relative | Mean | Mean | | value vs | (p value |
| | | staining | Marked | Area | area * | sity/ | (stimu- | to stimu- | per | per | | stimu- | vs stimu- |
| | | inten- | area | ROI | Inten- | Total | lated | lated | RHE | condition | | lated | lated + |
| | | sity | [μm²] | [μm²] | sity | area ROI | control) | control | (%) | (%) | SD | control) | vehicle |
| Unstimulated | RHE1 | 64.2 | 1668.8 | 7966.7 | 107083 | 13.4 | | 402 | 356 | 231 | 108 | 0.111 | 0.025 |
| control | | 62.2 | 1192.5 | 7818.0 | 74208 | 9.5 | | 284 | | | | | |
| | | 62.8 | 1659.0 | 8132.3 | 104116 | 12.8 | | 383 | | | | | |
| | RHE2 | 55.2 | 1174.2 | 9707.0 | 64836 | 6.7 | | 200 | 170 | | | | |
| | | 50.7 | 854.5 | 11189.0 | 43348 | 3.9 | | 116 | | | | | |
| | | 52.8 | 1087.2 | 8855.9 | 57352 | 6.5 | | 194 | | | | | |
| | RHE3 | 54.6 | 706.1 | 10069.7 | 38584 | 3.8 | | 114 | 168 | | | | |
| | | 65.0 | 968.7 | 10483.8 | 62992 | 6.0 | | 180 | | | | | |
| | | 57.9 | 1059.6 | 8774.5 | 61330 | 7.0 | | 209 | | | | | |
| Stimulated | RHE1 | 40.6 | 188.1 | 9637.7 | 7629 | 0.8 | 3.3 | 24 | 113 | 100 | 26 | 1.000 | 0.005 |
| control | | 53.2 | 779.5 | 6981.5 | 41475 | 5.9 | | 178 | | | | | |
| | | 59.1 | 713.8 | 9100.3 | 42194 | 4.6 | | 139 | | | | | |
| | RHE2 | 50.1 | 337.5 | 8688.4 | 16912 | 1.9 | | 58 | 117 | | | | |
| | | 61.2 | 762.4 | 8899.1 | 46691 | 5.2 | | 157 | | | | | |
| | | 55.4 | 679.0 | 8217.8 | 37638 | 4.6 | | 137 | | | | | |
| | RHE3 | 46.7 | 535.4 | 8064.9 | 24985 | 3.1 | | 93 | 70 | | | | |
| | | 62.6 | 228.5 | 8134.6 | 14313 | 1.8 | | 53 | | | | | |
| | | 52.1 | 338.6 | 8319.0 | 17646 | 2.1 | | 63 | | | | | |
| Stimulated + | RHE1 | 28.9 | 2.6 | 9909.9 | 75 | 0.0 | | 0 | 1 | 11 | 9 | 0.005 | 1.000 |
| vehicle | | 0.0 | 0.0 | 9585.5 | 0 | 0.0 | | 0 | | | | | |
| | | 41.0 | 11.5 | 10366.6 | 470 | 0.0 | | 1 | | | | | |
| | RHE2 | 41.5 | 226.7 | 10207.6 | 9417 | 0.9 | | 28 | 18 | | | | |
| | | 40.7 | 232.6 | 11838.5 | 9470 | 0.8 | | 24 | | | | | |
| | | 29.1 | 18.9 | 12804.8 | 551 | 0.0 | | 1 | | | | | |
| | RHE3 | 0.0 | 0.0 | 9824.1 | 0 | 0.0 | | 0 | 15 | | | | |
| | | 27.4 | 0.3 | 11632.9 | 9 | 0.0 | | 0 | | | | | |
| | | 44.3 | 359.5 | 10272.7 | 15926 | 1.6 | | 46 | | | | | |
| Stimulated + | RHE1 | 62.9 | 765.2 | 11042.5 | 48121 | 4.4 | | 130 | 169 | 149 | 25 | 0.082 | 0.001 |
| BETi1 1% | | 61.4 | 1106.0 | 10274.7 | 67964 | 6.6 | | 198 | | | | | |
| | | 65.1 | 1037.5 | 11327.5 | 67556 | 6.0 | | 178 | | | | | |
| | RHE2 | 43.8 | 408.3 | 9926.1 | 17884 | 1.8 | | 54 | 120 | | | | |
| | | 59.5 | 1056.5 | 10609.8 | 62851 | 5.9 | | 177 | | | | | |
| | | 47.2 | 951.2 | 10334.5 | 44943 | 4.3 | | 130 | | | | | |
| | RHE3 | 63.8 | 830.9 | 11896.8 | 53000 | 4.5 | | 133 | 158 | | | | |
| | | 57.8 | 1111.0 | 10214.3 | 64259 | 6.3 | | 188 | | | | | |
| | | 60.5 | 858.5 | 10159.6 | 51930 | 5.1 | | 153 | | | | | |
| Stimulated + | RHE1 | 51.3 | 855.0 | 11011.1 | 43824 | 4.0 | | 119 | 154 | 92 | 54 | 0.824 | 0.064 |
| BETi1 0.1% | | 57.2 | 803.1 | 11199.2 | 45938 | 4.1 | | 123 | | | | | |
| | | 60.9 | 1284.1 | 10557.5 | 78146 | 7.4 | | 221 | | | | | |
| | RHE2 | 60.5 | 449.4 | 10737.1 | 27165 | 2.5 | | 76 | 64 | | | | |
| | | 44.0 | 777.1 | 10052.9 | 34209 | 3.4 | | 102 | | | | | |
| | | 40.0 | 158.5 | 12283.3 | 6346 | 0.5 | | 15 | | | | | |
| | RHE3 | 55.1 | 363.9 | 11023.3 | 20044 | 1.8 | | 54 | 57 | | | | |
| | | 56.9 | 695.3 | 11573.6 | 39594 | 3.4 | | 102 | | | | | |
| | | 36.6 | 140.3 | 11200.6 | 5130 | 0.5 | | 14 | | | | | |
| Stimulated + | RHE1 | 50.7 | 236.2 | 12329.9 | 11983 | 1.0 | | 29 | 22 | 15 | 11 | 0.007 | 0.646 |
| BETi1 0.01% | | 61.1 | 198.9 | 10418.3 | 12156 | 1.2 | | 35 | | | | | |
| | | 32.8 | 6.9 | 10745.5 | 227 | 0.0 | | 1 | | | | | |
| | RHE2 | 27.5 | 0.1 | 10810.4 | 2 | 0.0 | | 0 | 22 | | | | |
| | | 60.9 | 310.6 | 10475.9 | 18906 | 1.8 | | 54 | | | | | |
| | | 43.1 | 104.1 | 11909.8 | 4484 | 0.4 | | 11 | | | | | |
| | RHE3 | 39.7 | 73.9 | 11528.2 | 2936 | 0.3 | | 8 | 3 | | | | |
| | | 29.8 | 1.8 | 10043.2 | 55 | 0.0 | | 0 | | | | | |
| | | 29.8 | 1.8 | 10043.2 | 55 | 0.0 | | 0 | | | | | |
| Stimulated + | RHE1 | 44.9 | 220.6 | 9321.0 | 9896 | 1.1 | | 32 | 18 | 17 | 3 | 0.006 | 0.343 |
| BETi1 10.001% | | 54.9 | 145.6 | 10966.5 | 7998 | 0.7 | | 22 | | | | | |
| | | 30.6 | 3.1 | 9257.7 | 94 | 0.0 | | 0 | | | | | |
| | RHE2 | 28.6 | 3.1 | 10655.4 | 88 | 0.0 | | 0 | 19 | | | | |
| | | 77.7 | 187.8 | 11411.9 | 14591 | 1.3 | | 38 | | | | | |
| | | 50.8 | 153.2 | 12080.8 | 7778 | 0.6 | | 19 | | | | | |
| | RHE3 | 50.2 | 132.7 | 12409.9 | 6661 | 0.5 | | 16 | 14 | | | | |
| | | 39.7 | 61.0 | 10824.0 | 2423 | 0.2 | | 7 | | | | | |
| | | 37.3 | 249.6 | 13752.4 | 9304 | 0.7 | | 20 | | | | | |

TABLE 21
MMP-9 secretion (% of stimulated CTL) relative to the quantification of MMP-9 in culture medium
of RHE stimulated or not with TNF-α/IFN-γ and treated with 6 formulations vs 1 placebo for 48 h

| | | | | | Mean | | | | | | | Student t- | |
|---|---|---|---|---|---|---|---|---|---|---|---|---|---|
| | | Conc MMP-9 | | | conc | | MMP-9 (% | | | | | test vs | Student t- |
| | | (ng/ml) | | | MMP-9 | | stimulated ctl) | | | | | Stimulated | test vs |
| Conditions | | N1 | N2 | N3 | (ng/ml) | SD | N1 | N2 | N3 | Mean | SD | CTL | placebo |
| Ø TNF-α/ | Untreated | 3.1 | 2.5 | 2.7 | 2.7 | 0.3 | 17 | 14 | 15 | 16 | 1.7 | 0.00000658 | 1.4264E−06 |
| IFN-γ | CTL | | | | | | | | | | | | |
| TNF-α | Stimulated | 16.7 | 17.6 | 18.3 | 17.6 | 0.8 | 95 | 100 | 104 | 100 | 4.4 | — | 0.00039961 |
| 12 ng/ml/ | CTL | | | | | | | | | | | | |
| IFN-γ | Placebo | 24.4 | 25.2 | 23.7 | 24.4 | 0.8 | 139 | 144 | 135 | 139 | 4.4 | 0.00039961 | — |
| 12 ng/ml | BETi1 - 1% | 0.9 | 1.1 | 0.8 | 0.9 | 0.1 | 5 | 6 | 5 | 5 | 0.8 | 0.00000330 | 8.2113E−07 |
| | BETi1- 0.1% | 1.5 | 2.0 | 1.6 | 1.7 | 0.3 | 9 | 12 | 9 | 10 | 1.5 | 0.00000477 | 1.1214E−06 |
| | BETi1 - 0.01% | 19.9 | 18.2 | 21.4 | 19.8 | 1.6 | 113 | 104 | 122 | 113 | 9.1 | 0.08977988 | 0.01068173 |
| | BETi1 - 0.001% | 24.5 | 24.1 | 23.5 | 24.1 | 0.5 | 140 | 138 | 134 | 137 | 3.0 | 0.00027673 | 0.52415551 |
| | Rux 1.5% | 0.4 | 4.9 | 0.7 | 2.0 | 2.5 | 2 | 28 | 4 | 11 | 14.3 | 0.00050991 | 0.00012142 |

TABLE 22
E-Cadherin secretion (% of treated unstimulated CTL) relative to the quantification of E-Cadherin in culture
medium of RHE stimulated or not with TNF-α/IFN-γ and treated with 6 formulations vs 1 placebo for 48 h

| | | | | | Mean | | | | | | | Student | |
|---|---|---|---|---|---|---|---|---|---|---|---|---|---|
| | | Conc E-Cad | | | conc | | E-Cad (% | | | | | t-test vs | Student |
| | | (ng/ml) | | | E-Cad | | stimulated ctl) | | | | | Stimulated | t-test |
| Conditions | | N1 | N2 | N3 | (ng/ml) | SD | N1 | N2 | N3 | Mean | SD | CTL | vs placebo |
| Ø TNF-α/ | Untreated | 0.7 | 0.8 | 0.7 | 0.8 | 0.0 | 32 | 34 | 32 | 33 | 1.1 | 0.00005776 | 0.000368 |
| IFN-γ | CTL | | | | | | | | | | | | |
| TNF-α | Stimulated | 2.3 | 2.4 | 2.1 | 2.3 | 0.1 | 102 | 105 | 93 | 100 | 6.4 | — | 0.3908565 |
| 12 ng/ml/ | CTL | | | | | | | | | | | | |
| IFN-γ | Placebo | 2.7 | 2.2 | 2.5 | 2.5 | 0.3 | 118 | 95 | 110 | 107 | 11.5 | 0.39085655 | — |
| 12 ng/ml | BETi1 - 1% | 1.6 | 1.7 | 1.7 | 1.7 | 0.0 | 71 | 72 | 74 | 72 | 1.1 | 0.00180438 | 0.006311 |
| | BETi1- 0.1% | 1.4 | 1.3 | 1.4 | 1.4 | 0.1 | 61 | 56 | 62 | 59 | 3.4 | 0.00062760 | 0.0022853 |
| | BETi1 - 0.01% | 2.7 | 2.5 | 2.9 | 2.7 | 0.2 | 117 | 109 | 128 | 118 | 9.3 | 0.05288926 | 0.2890528 |
| | BETi1 - 0.001% | 3.0 | 3.0 | 3.6 | 3.2 | 0.4 | 129 | 129 | 156 | 138 | 15.4 | 0.01678353 | 0.0501819 |
| | Rux 1.5% | 1.8 | 5.7 | 3.6 | 3.7 | 1.9 | 77 | 247 | 155 | 160 | 84.7 | 0.28975076 | 0.3476291 |

TABLE 23
Comparison of Stimulated RHE vs unstimulated RHE (untreated)

| | GENE | | | P- |
|---|---|---|---|---|
| | SYMBOLS | GENE NAMES | FC | VALUES |
| Up- | IL6 | Interleukin 6 | 25.7371 | 0.0005 |
| regulations | IL1A | Interleukin 1 alpha | 10.4477 | 0.016 |
| | IL1B | Interleukin 1 beta | 10.2706 | 0.0048 |
| | TNF | Tumor necrosis | 8.1179 | 0.038 |
| | | factor/TNF | | |
| | | superfamily, member 2 | | |
| | SOX9 | SRY (sex determining | 5.591 | 0.0029 |
| | | region-Y)-box 9 | | |
| | CASP8 | Caspase-8, | 5.0611 | 0.0056 |
| | | apoptosis-related | | |
| | | cysteine peptidase | | |
| | EDN1 | Endothelin 1 | 4.3113 | 0.0078 |
| | POMC | Proopiomelanocortin | 3.8038 | 0.0096 |
| | DVL1 | Dishevelled, | 3.0982 | 0.0433 |
| | | dsh homolog 1 | | |
| | USF1 | Upstream transcription | 2.9372 | 0.0123 |
| | | factor 1 | | |
| | RAC1 | Ras-related C3 botulinum | 2.0889 | 0.0416 |
| | | toxin substrate 1 | | |
| | CREBBP | CREB binding protein | 1.9231 | 0.0356 |
| | STX6 | Syntaxin 6 | 1.9112 | 0.024 |
| | AIM1 | Absent in melanoma 1 | 1.9032 | 0.0444 |
| | KIT | Mast/stem cell | 1.7292 | 0.038 |
| | | growth factor | | |
| | | receptor or Proto- | | |
| | | oncogene c-KIT | | |
| Down- | FGFR2 | Fibroblast growth factor | 0.6391 | 0.0463 |
| regulations | | receptor 2 | | |
| | WNT16 | Wingless-type MMTV | 0.5095 | 0.0416 |
| | | integration site family, | | |
| | | member 16 | | |
| | RAB3A | Ras-related protein | 0.4317 | 0.0126 |
| | | Rab-3A | | |
| | PRKCB | Protein kinase C beta | 0.3666 | 0.0279 |

TABLE 24
Comparison of Stimulated RHE treated with the vehicle
(BETi1 vehicle) vs stimulated RHE (untreated)

| | GENE | | | P- |
|---|---|---|---|---|
| | SYMBOLS | GENE NAMES | FC | VALUES |
| Up | GADD45A | Growth arrest and DNA- | 3.3553 | 0.007 |
| | | damage-inducible, alpha | | |
| | SLC24A5 | Solute carrier family 24, | 1.5036 | 0.0092 |
| | | member 5 | | |
| | STX6 | Syntaxin 6 | 2.0187 | 0.0265 |
| Down | WNT16 | Wingless-type MMTV | 0.2964 | 0.0293 |
| | | integration site family, | | |
| | | member 16 | | |
| | RAB3A | Ras-related protein Rab-3A | 0.5654 | 0.0416 |

TABLE 25
Comparison of Stimulated RHE treated with BETi1 at 1% vs
stimulated RHE treated with the vehicle (BETi1 vehicle)

| | | Stimulated + BETi1 1% vs Stimulated + BETi1 vehicle | | | | |
|---|---|---|---|---|---|---|
| | | | | | Results as | |
| | | | Results | | originally | |
| | | | after correction | | presented** | |
| Gene symbols | | Gene names | FC | P-Values | FC | P-Value |
| Up- | RAB3A | RAB3A, member | 14.5685 | 0 | 14.5685 | 0 |
| regulation | | RAS oncogene family | | | | |
| | WNT16 | Wnt family member | 10.0801 | 0.0095 | 10.0801 | 0.0095 |
| | | 16 | | | | |
| | PCNA | proliferating cell | 7.0134 | 0.0265 | 7.0134 | 0.0265 |
| | | nuclear antigen | | | | |
| | XPC | Xeroderma | 1.6569 | 0.0455 | 1.8367 | 0.039 |
| | | pigmentosum, | | | | |
| | | complementation | | | | |
| | | group C | | | | |
| | KIT | Mast/stem cell growth | 0.6404 | 0.0206 | 1.6569 | 0.0455 |
| | | factor receptor or | | | | |
| | | Proto-oncogene c-KIT | | | | |
| Down- | AP1S1 | Adaptor-related | 0.6116 | 0.0084 | 0.727 | 0.0403 |
| regulations | | protein complex 1, | | | | |
| | | sigma 1 subunit | | | | |
| | BCL2 | B-cell lymphoma 2 | 0.5772 | 0.0157 | 0.6404 | 0.0206 |
| | HPS1 | Hermansky-Pudlak | 0.5127 | 0.0431 | 0.6116 | 0.0084 |
| | | syndrome 1 | | | | |
| | USF1 | Upstream | 0.497 | 0.0245 | 0.5772 | 0.0157 |
| | | transcription factor 1 | | | | |
| | ASIP* | Agouti signaling | 0.4753 | 0.0269 | 0.5127 | 0.0431 |
| | | protein | | | | |
| | CALML6* | Calmodulin-like 6 | 0.4753 | 0.0269 | 0.497 | 0.0245 |
| | CAMK2A* | Calcium/calmodulin- | 0.4753 | 0.0269 | 0.4753 | 0.0269 |
| | | dependent protein | | | | |
| | | kinase II alpha | | | | |
| | GNAO1* | G Protein Subunit | 0.4753 | 0.0269 | 0.4753 | 0.0269 |
| | | Alpha O1 | | | | |
| | PTGER1* | Prostaglandin E | 0.4753 | 0.0269 | 0.4753 | 0.0269 |
| | | receptor 1 | | | | |
| | MYO5A | Myosin VA | 0.4524 | 0.0215 | 0.4753 | 0.0269 |
| | AIM1 | Absent in melanoma 1 | 0.4502 | 0.0343 | 0.4753 | 0.0269 |
| | DVL1 | Dishevelled, dsh | 0.4492 | 0.0405 | 0.4524 | 0.0215 |
| | | homolog 1 | | | | |
| | GNAS | Adenylate cyclase- | 0.4273 | 0.0484 | 0.4502 | 0.0343 |
| | | stimulating G alpha | | | | |
| | | protein | | | | |
| | PTGER3 | Prostaglandin E | 0.4259 | 0.0007 | 0.4492 | 0.0405 |
| | | receptor 3 | | | | |
| | ATP7A | ATPas, Cu++ | 0.4094 | 0.039 | 0.4273 | 0.0484 |
| | | transporting, alpha | | | | |
| | | polypeptide | | | | |
| | TYR | Tyrosinase | 0.405 | 0.0228 | 0.4259 | 0.0007 |
| | SOX10 | SRY-box 10 | 0.4034 | 0.0246 | 0.4094 | 0.039 |
| | MAPK14 | Mitogen-activated | 0.3927 | 0.011 | 0.405 | 0.0228 |
| | | protein kinase 14 | | | | |
| | STX6 | Syntaxin 6 | 0.3683 | 0.0182 | 0.4034 | 0.0246 |
| | GNAI1 | Adenylate cyclase- | 0.3672 | 0.0093 | 0.3927 | 0.011 |
| | | inhibiting G alpha | | | | |
| | | protein | | | | |
| | RAB38 | RAB38, member RAS | 0.3579 | 0.0016 | 0.3683 | 0.0182 |
| | | oncogene family | | | | |
| | CREBBP | CREB binding protein | 0.3195 | 0.0229 | 0.3672 | 0.0093 |
| | HPS4 | Hermansky-Pudlak | 0.3088 | 0.0014 | 0.3579 | 0.0016 |
| | | syndrome 4 | | | | |
| | MLPH | Melanophilin | 0.3039 | 0.031 | 0.3195 | 0.0229 |
| | OCA2 | Oculocutaneous | 0.293 | 0.0161 | 0.3088 | 0.0014 |
| | | albinism II | | | | |
| | RAB27A | RAB27A, member | 0.2763 | 0.0154 | 0.3039 | 0.031 |
| | | RAS oncogene family | | | | |
| | ATP2A2 | ATPase, Ca++ | 0.2632 | 0.0028 | 0.293 | 0.0161 |
| | | transporting, cardiac | | | | |
| | | muscle, slow twitch 2 | | | | |
| | BAD | Bcl2-associated | 0.2547 | 0.0098 | 0.2763 | 0.0154 |
| | | agonist of cell death | | | | |
| | GADD45A | Growth arrest and | 0.2375 | 0.0054 | 0.2632 | 0.0028 |
| | | DNA-damage- | | | | |
| | | inducible, alpha | | | | |
| | SNAP25 | Synaptosomal- | 0.2366 | 0.0316 | 0.2547 | 0.0098 |
| | | associated protein, | | | | |
| | | 25 kDa | | | | |
| | EDNRB | endothelin receptor | 0.1544 | 0.0051 | 0.2375 | 0.0054 |
| | | type B | | | | |
| | RAB32 | RAB32, member RAS | 0.1471 | 0.0032 | 0.2366 | 0.0316 |
| | | oncogene family | | | | |
| | SLC24A5 | solute carrier family | 0.1051 | 0.0007 | 0.1544 | 0.0051 |
| | | 24 member 5 | | | | |
| | POMC | proopiomelanocortin | 0.1039 | 0.0105 | 0.1471 | 0.0032 |
| | SOX9 | SRY-box 9 | 0.0882 | 0.0062 | 0.1051 | 0.0007 |
| | EDN1 | endothelin 1 | 0.0395 | 0.0199 | 0.1039 | 0.0105 |
| | IL1B | Interleukin 1 beta | 0.0208 | 0.0002 | 0.0882 | 0.0062 |
| | IL1A | Interleukin 1 alpha | 0.0124 | 0.0001 | 0.0395 | 0.0199 |

*Results for these particular genes are inconclusive as Ct values for some replicates were above 36.
**Results for certain genes in this table were initially incorrectly presented by the contract research organization (CRO) due to a transcription error. Both the results as originally presented (with a transcription error for some genes) and the corrected results (without transcription error) are presented for sake of completeness.

TABLE 26
Comparison of Stimulated RHE treated with BETi1 at 0.1% vs
stimulated RHE treated with the vehicle (BETi1 vehicle)

| | GENE | | | P- |
|---|---|---|---|---|
| | SYMBOLS | GENE NAMES | FC | VALUES |
| Up- | RAB3A | RAB3A, member RAS | 10.8195 | 0.0222 |
| regulation | | oncogene family | | |
| | WNT16 | Wnt family member 16 | 8.7922 | 0.0057 |
| | PCNA | proliferating cell | 3.2615 | 0.0117 |
| | | nuclear antigen | | |
| | KITLG | Xeroderma pigmentosum, | 1.8415 | 0.0349 |
| | | complementation group C | | |
| | PPIA | Peptidylpropyl | 1.6969 | 0.048 |
| | | isomerase A | | |
| Down | AP1M1 | Adaptor-related protein | 0.6311 | 0.0076 |
| regulation | | complex 1, mu 1 subunit | | |
| | GNAI1 | Adenylate cyclase- | 0.5815 | 0.0248 |
| | | inhibiting G alpha protein | | |
| | RAB38 | RAB38, member RAS | 0.5643 | 0.0445 |
| | | oncogene family | | |
| | MYO5A | Myosin VA | 0.5462 | 0.0337 |
| | CALML6 | Calmodulin-like 6 | 0.5429 | 0.0066 |
| | CAMK2A | Calcium/calmodulin- | 0.5043 | 0.0207 |
| | | dependent protein | | |
| | | kinase II alpha | | |
| | PTGER3 | Prostaglandin E receptor 3 | 0.4799 | 0.0048 |
| | MAPK14 | Mitogen-activated | 0.4724 | 0.0162 |
| | | protein kinase 14 | | |
| | HPS4 | Hermansky-Pudlak | 0.4224 | 0.0426 |
| | | syndrome 4 | | |
| | STX6 | Syntaxin 6 | 0.4214 | 0.0169 |
| | SOX10 | SRY-box 10 | 0.4196 | 0.0424 |
| | MLPH | Melanophilin | 0.355 | 0.0341 |
| | ATP2A2 | ATPase, Ca++ | 0.2769 | 0.0261 |
| | | transporting, | | |
| | | cardiac muscle, | | |
| | | slow twitch 2 | | |
| | EDNRB | Endothelin receptor type B | 0.2737 | 0.0173 |
| | BAD | Bcl2-associated | 0.2735 | 0.0108 |
| | | agonist of cell death | | |
| | SLC24A5 | Solute carrier family | 0.272 | 0.0498 |
| | | 24, member 5 | | |
| | RAB32 | RAB 32, member | 0.2378 | 0.0111 |
| | | RAS oncogene family | | |
| | GADD45A | Growth arrest and DNA- | 0.1803 | 0.002 |
| | | damage-inducible, alpha | | |
| | POMC | Proopiomelanocortin | 0.1665 | 0.0322 |
| | SOX9 | SRY-box 9 | 0.1155 | 0.0098 |
| | ADCY2 | Adenylate cyclase-2 | 0.1137 | 0.0187 |
| | IL1B | Interleukin 1 beta | 0.0727 | 0.0065 |
| | IL1A | Interleukin 1 alpha | 0.0454 | 0.0022 |
| | EDN1 | Endothelin 1 | 0.0413 | 0.0169 |

TABLE 27
Comparison of Stimulated RHE treated with BETi1 at 0.01% vs
stimulated RHE treated with the vehicle (BETi1 vehicle)

| | GENE | | | P- |
|---|---|---|---|---|
| | SYMBOLS | GENE NAMES | FC | VALUES |
| up | RAB3A | RAB3A, member | 3.5532 | 0.0222 |
| | | RAS oncogene family | | |
| | AP1S1 | Adaptor-related protein | 1.3312 | 0.0283 |
| | | complex 1, sigma 1 subunit | | |
| Down- | ASIP | Agouti signaling protein | 0.6303 | 0.0219 |
| regulations | CALML6 | Calmodulin-like 6 | 0.6303 | 0.0219 |
| | CAMK2A | Calcium/calmodulin- | 0.6303 | 0.0219 |
| | | dependent protein | | |
| | | kinase II alpha | | |
| | GNAO1 | G Protein Subunit Alpha O1 | 0.6303 | 0.0219 |
| | POMC | Proopiomelanocortin | 0.4147 | 0.0035 |
| | GADD45A | Growth arrest and DNA- | 0.3574 | 0.0248 |
| | | damage-inducible, alpha | | |

TABLE 28
Comparison of Stimulated RHE treated with BETi1 at 0.001%
vs stimulated RHE treated with the vehicle (BETi1 vehicle)

| | | Stimulated + BETi1 - 0.001% vs Stimulated + BETi1 vehicle | | | | |
|---|---|---|---|---|---|---|
| | | | Results | | Results as originally | |
| | Gene | | after correction | | presented** | |
| | symbols | Gene names | FC | P-Values | FC | P-Values |
| up | APAF1 | Apoptotic peptidase | 1.5797 | 0.0316 | 3.5532 | 0.0222 |
| | | activating factor-1 | | | | |
| | BCL2 | B-cell lymphoma 2 | 0.7462 | 0.0347 | 1.3312 | 0.0283 |
| Down- | AP1M1 | Adaptor-related | 0.7447 | 0.0387 | 0.6303 | 0.0219 |
| regulations | | protein complex 1, | | | | |
| | | mu 1 subunit | | | | |
| | ASIP* | Agouti signaling | 0.7104 | 0.0251 | 0.6303 | 0.0219 |
| | | protein | | | | |
| | CALML6* | Calmodulin-like 6 | 0.7104 | 0.0251 | 00,272.6303 | 0.0219 |
| | GNAO1* | G Protein Subunit | 0.7104 | 0.0251 | 0.6303 | 0.0219 |
| | | Alpha 01 | | | | |
| | HPS4 | Hermansky-Pudlak | 0.6711 | 0.0301 | 0.4147 | 0.0035 |
| | | syndrome 4 | | | | |
| | POMC | Proopiomelanocortin | 0.5586 | 0.0019 | 0.3574 | 0.0248 |

*Results for these particular genes are inconclusive as Ct values for some replicates were above 36.
**Results for genes in this table were initially incorrectly presented due to a transcription error by the CRO. Both the results as originally presented (with a transcription error) and the corrected results (without transcription error) are presented for sake of completeness.

TABLE 29
Comparison of Stimulated RHE treated with Ruxolitinib cream
at 1.5% vs stimulated untreated RHE

| | GENE | | | P- |
|---|---|---|---|---|
| | SYMBOLS | GENE NAMES | FC | VALUES |
| Up- | MAP2K1 | Mitogen-activated protein | 4.6701 | 0.0017 |
| regulations | | kinase kinase 1 | | |
| | SOX9 | SRY-box9 | 3.8981 | 0.0005 |
| | GADD45A | Growth arrest and DNA- | 3.8179 | 0.0102 |
| | | damage-inducible, alpha | | |
| | FZD10 | Frizzled family receptor 10 | 3.6044 | 0.0386 |
| | BCL2 | B-cell lymphoma 2 | 2.8444 | 0.0275 |
| | SNAP23 | Synaptosomal-associated | 1.8164 | 0.0137 |
| | | protein, 23 kDa | | |
| | EDN1 | Endothelin-1 | 1.5455 | 0.0442 |
| Down- | DTNBP1 | Dystrobrevin | 0.5074 | 0.0472 |
| regulations | | binding protein 1 | | |
| | HPS3 | Hermansky-Pudlak | 0.4108 | 0.0062 |
| | | symdrome 3 | | |
| | PPIA | Peptidylpropyl isomerase A | 0.3868 | 0.0413 |
| | HPS4 | Hermansky-Pudlak | 0.3443 | 0.0226 |
| | | syndrome 4 | | |
| | PRKACA | Protein kinase, cAMP- | 0.3136 | 0.0138 |
| | | dependnet, catalytic, alpha | | |
| | RAB3A | Ras-related protein Rab-3A | 0.3077 | 0.012 |
| | CASP8 | Caspase-8, apoptosis- | 0.3067 | 0.0134 |
| | | related cysteine peptidase | | |
| | FGFR2 | Fibroblast-growth | 0.2995 | 0.0436 |
| | | factor receptor 2 | | |
| | TYR | Tyrosinase | 0.2896 | 0.0176 |
| | GNAI1 | Adenylate cyclase- | 0.1838 | 0.0002 |
| | | inhibiting G alpha protein | | |
| | POMC | Bcl2-associated | 0.1218 | 0.0276 |
| | | agonist of cell death | | |
| | RAB27A | RAB27A, Member | 0.1006 | 0.0362 |
| | | RAS Oncogene Family | | |
| | PTGER3 | Prostaglandin E | 0.0945 | 0.0235 |
| | WNT16 | Wnt family member 16 | 0.0757 | 0.0009 |

### DISCUSSION

The above data shows the in vitro analysis of the effects of BETi1 at 4 concentrations on reconstituted human melanized epidermis (RHE-MWL) stimulated with a cytokine cocktail (TNF-α and IFN- [symbol] ) in order to induce melanocytorhagy and feature vitiligo. The effects of the formulation BETi 1 were compared to a placebo (BETi 1—vehicle) used as base for the preparation of the formulations. In parallel, another formulation, Ruxolitinib cream at 1.500 was used as reference. Ruxolitinib is a JAK1/2 inhibitor (JAK-STAT pathway being involved in the signalization cascade of IFN- [symbol] ) already used in clinical assays for the treatment of vitiligo.

The effects of the formulations were assessed on different parameters, firstly on the release of MMNP9 and E-cadherin in culture medium of RHE-MEL which are characteristic of the disease, secondly on the expression of TRP-1 (a specific marker of melanocytes) in the basal layers, and thirdly on melanin abundance in the basal layers and finally on the expression of 92 genes involved in melanogenesis.

It was found that the BETi11 formulation at 1% was able to reverse to a considerable extent the effects of the stimulation with the cytokine cocktail on the different parameters analyzed. The BETi1 formulation at 0.1% was effective as well, but to a lesser extent and not on all the parameters. Table 30 below synthetizes the effects of the formulations on the parameters analyzed in the vitiligo model.

TABLE 30

| | | | E- | TRP-1 | Melanin |
|---|---|---|---|---|---|
| | | MMP9 | cadherin | expression | content in |
| | | release | release | in basal | basal |
| | | from | from | layers of | layers of |
| | | RHE | RHE | RHE | RHE |
| Effects by | Stimulated | [symbol] | [symbol] | [symbol] | [symbol] |
| comparison | control, | | | | |
| with un- | untreated | | | | |
| stimulated | | | | | |
| control | | | | | |
| Effects by | Stimulated + | [symbol] | [symbol] | [symbol] [symbol] ( [symbol] ) | [symbol] |
| comparison | BETi1 − 1% | | | | |
| with | Stimulated + | [symbol] | [symbol] | — | [symbol] |
| Stimulated+ | BETi1 − 0.1% | | | | |
| BETi1 − | Stimulated + | [symbol] | — | — | — |
| vehicle | BETi1 − | | | | |
| | 0.01% | | | | |
| | Stimulated + | — | [symbol] | — | [symbol] [symbol] ( [symbol] ) |
| | BETi1 − | | | | |
| | 0.001% | | | | |
| Effects by | Stimulated + | [symbol] | — | NA | NA |
| comparison | Ruxolitinib | | | | |
| with | cream | | | | |
| stimulated | 1.5% | | | | |
| control | | | | | |

The significant up-regulations are presented as [symbol] and the significant down-regulations as [symbol] , with ( [symbol] or [symbol] ): 0.05 > p > 0.01; ( [symbol] or [symbol] ): 0.01 > p > 0.001; and ( [symbol] or [symbol] ): p < 0.001. NA stands for not applicable.

The BETi1 formulation at 1% (and to a lesser extent at 0.1%) was thus effective to prevent/reduce the melanocytorhagy process as depicted by the location of TRP-1 protein and of melanin in the epidermis (present in the basal layers). The formulation was also able to reduce the release of MMP9 and E-cadherin that occurs during melanocytorhagy. No conclusion could be drawn about the effects of the ruxolitinib cream at 1.5%, given to the morphology alterations that were observed following its application.

Regarding the gene expression analysis, the main genic regulations observed after the stimulation with the cytokine cocktail concerned the induction of inflammatory and stress response genes (e.g., interleukins 1A and 1B, TNF, CASP8). Noteworthy, the TNF-α/IFN-γ cocktail also decreased the expression of WNT16, belonging to the Wnt/β-catenin pathway (one of the main pathways leading to the activation of MITF, a key transcription factor involved in the melanocytes differentiation, growth and survival) known to be altered in vitiligo. Other genes of the melanocyte biology were also impacted with some regulations in favor and others in disfavor of the melanogenesis process. The vehicle used in the study as base for the preparation of the BETi1 formulation did not have major effects on gene expression. Nevertheless, it was noted the induction of GADD45A gene, a marker of DNA damage and the downregulation of WNT16 which might be not beneficial for the melanocytes, given the vitiligo context. It is not known why the vehicle comprising petrolatum had some effect on gene expression but without being bound by any theory it may possibly be related to an occlusive effect on the RHE.

The BETi1 formulations at 1% and 0.1% were able to reverse many gene regulations induced by the cytokine cocktail. It is the case among others for IL1A, IL1B, SOX9, POMC and EDN1 (increased with the cytokine cocktail and decreased with the BETi1 formulation) and for WNT16 and RAB3A (decreased with the cytokine cocktail and increased in presence of the formulation). The various Examples in this in vitro study showed that BETi1 at 1% (and to a lesser extent at 0.1%) is an excellent candidate for the prevention of melanocytorhagy in the treatment of vitiligo.

The various Examples described herein accordingly illustrate that the PAN BETi compounds described herein can be useful and beneficial in the treatment, prevention and amelioration of pigmentation diseases and disorders, particularly those involving a lightening of color and/or appearance of pale or white skin patches.

### Section C:

### Experimental Method N: Preparation of Formulations Used in Method J

### A. Preparation of Anti-Collagen mAb

mAb stock solution (10 mg/ml), supplied by MD Biosciences, Division of Morwell Diagnostics GmbH, is used as provided. Each mouse is injected intravenous (IV) with 400 μl (4 mg) of anti-collagen mAb.

### B. Preparation of LPS

LPS is diluted with PBS to achieve an appropriate concentration of 0.25 mg/ml. Thorough vertexing is required just before its injection.

### C. Preparation of BETi1 Suspensions and Vehicle

TABLE 31

| | Formulation | Formulation | Formulation | Formulation | Vehicle |
|---|---|---|---|---|---|
| | #1 | #2 | #3 | #4 | #5 |
| | % w/w | | | | |
| BETi1 | 0.4 | 0.04 | 0.004 | 0.0004 | 0 |
| Na | 0.5 | 0.5 | 0.5 | 0.5 | 0.5 |
| CMC | | | | | |
| NaCl | 0.9 | 0.9 | 0.9 | 0.9 | 0.9 |
| Water | 98.2 | 98.56 | 98.596 | 98.5996 | 98.6 |

BETi1 is dispersed in 0.5% Sodium Carboxymethyl Cellulose (NaCMC) solution in NaCl saline (0.9% NaCl in water) to receive 4 mg/gram dispersion. A homogenous dispersion is accomplished by sonication at 40 kHz for 30 minutes. The homogeneity including absence of aggregates of particles of the dispersion is confirmed by light microscopy. Lower concentrations are obtained by corresponding dilution of dispersion at 4 mg/gram with NaCMC solution. All dispersions are thermally sterilized.

Animals weighing −20 g are administered with 0.2 ml or 50 μl of dosing solution via IA (Groups 1-5). Formulations 1-5 are prepared as set out below to obtain dose levels (mg/kg) as set out in Table 32A and are dosed on study days 0, 3, 6 and 9 as set out in Table B.
I. 10 mg/kg BETi1 injected via IA to group 2: 4 mg/mL suspension in 0.5% NaCMC and saline (Formulation #1)—to provide approx. 200 μg in 50 μL.
II. 1 mg/kg BETi1 injected via IA to group 3: 0.4 mg/mL suspension in 0.5% NaCMC and saline (Formulation #2)—to provide approx. 20 μg in 50 μL.
III. 0.1 mg/kg BETi1 injected via IA group 4: 0.04 mg/mL suspension in 0.5% NaCMC and saline (Formulation #3)—to provide approx. 2 μg in 50 μL.
IV. 0.01 mg/kg BETi1 injected via IA to group 5: 0.004 mg/mL suspension in 0.5% NaCMC and saline (Formulation #4)—to provide approx. 0.2 μg in 50 μL.
V. vehicle (Formulation #5) injected via IA to group 1: 0.5% NaCMC and saline is a clear solution and can be used without any additional preparation.

Formulations #1 through #3 are suspensions and are re-suspended prior to injection. The concentration of BETi in Formulation #4 is within the approximate solubility of BETi1 in water of about 0.005 mg/gram, and therefore most or all of the compounds may be dissolved in the formulation. Sonication for 10 minutes is used for re-suspension of the samples prior to use until it is visually homogeneous i.e. no residue on the bottom of the vial and no visible aggregates.

Even though the formulations are slightly viscous in order to keep API particles suspended, the sample should be taken from the vial as soon as possible after re-suspension to avoid sedimentation of API. The samples are stored at 5° C. prior to use, then equilibrated with RT and re-suspended.

D. Preparation of Dexamethasone at a Dose Level of 1 mg/Kg, at a Concentration of 0.1 mg/ml, and a Dose Volume of 10 ml/Kg (Groups 6 and 7)

Dexamethasone stock solution at a concentration of 1 mg/ml is prepared by mixing 5 mg of Dexamethasone until fully dissolved in 5 ml of absolute ethanol. Dexamethasone 0.1 mg/ml is prepared by adding 1.8 ml of PBS to 0.2 ml of stock solution and vortexing well until reaching homogeneity.

Animals weighing ˜20 g are administered with 0.2 ml or 50 μl of dosing solution via IP (Group 6) or for volume dosage of IA (Group 7) respectively. Animals are dosed on study days 0-12 (Group 6, IP) or on study days 0, 3, 6 and 9 (Group 7, IA) as set out in Tables A and B.

Experimental Method O: In Vivo Efficacy and Tolerance from a Murine Anti Collagen Antibodies Induced Arthritis (ACAIA) Mimicking Rheumatoid Arthritis and Treated with BETi 1 Formulations

During the acclimation period (at least 5 days), animals are randomly assigned to experimental groups according to body weight. Each dosing group is kept in separate cages to avoid cross-contamination which can occur through the consumption of fecal matter during the study. To generate the morphological and functional aspects of Rheumatoid Arthritis, all mice (7 mice in each of Groups 1-7) are subjected to a tail intravenous (IV) injection of 0.4 ml of anti-collagen mAb (4 mg in 400 μl) on study day 0. LPS is injected intraperitoneally on study day 4 (50 μg LPS in 200 μl of PBS). Anti-collagen mAb is supplied by MD Biosciences, Division of Morwell Diagnostics GmbH and is used as provided.

Balb/c. mice male −7 weeks old at study initiation weighing ˜20 g are administered with 50 μl of dosing solution (vehicle and BETi1 at four concentrations) via intra-articular (IA) administration. The control, dexamethasone, is diluted in ethanol to achieve a concentration of 1 mg/ml and diluted again in phosphate buffered saline (PBS) to achieve the dose concentration of 0.1 mg/ml as set out in Method A. 50 μl of the negative control is administered intraperitoneally (IP) on study days 0-12 (Group 6). 50 μl of the positive control is administered via intra-articular (IA) on study days 0, 3, 6 and 9 (Group 7).

Experimental groups and treatments are listed in Tables 32A and 32B respectively.

TABLE 32A

| Group | | | Dose Level | | Volume | | Disease | Test Items |
|---|---|---|---|---|---|---|---|---|
| Number | Test Material | Route | (mg/kg) | | Dosage | | induction | Regime |
| 1 | BETi 1 Vehicle | IA | N/A | | N/A | | 4 mg mAb in | On study days 0, |
| | (formulation #5) | | | | | | 400 μl | 3, 6 and 9 |
| 2 | BETI 1 | | 10 | mg/kg | 50 | μl | via IV onday 0 | |
| | 4 mg/mL | | | | | | 50 μg LPS in | |
| | Suspension | | | | | | 200 μl PBS | |
| | (formulation | | | | | | vialP on day 4 | |
| | #1) | | | | | | | |
| 3 | BETI 1 | | 1 | mg/kg | | | | |
| | 0.4 mg/mL | | | | | | | |
| | Suspension | | | | | | | |
| | (formulation | | | | | | | |
| | #2) | | | | | | | |
| 4 | BETi1 | | 0.1 | mg/kg | | | | |
| | 0.04 mg/mL | | | | | | | |
| | Suspension | | | | | | | |
| | (formulation | | | | | | | |
| | #3) | | | | | | | |
| 5 | BETi 1 | | 0.01 | mg/kg | | | | |
| | 0.004 mg/mL | | | | | | | |
| | suspension | | | | | | | |
| | (formulation | | | | | | | |
| | #4) | | | | | | | |
| 6 | (formulation #6) | IP | 1 | mg/kg | 5 | ml/kg | | Daily on study |
| | Dexamethasone | | | | | | | from day 0 until |
| | (formulation #7) | | | | | | | day 11 |
| 7 | | IA | | | 50 | μl | | On study days |
| | | | | | | | | 0, 3, 6 and 9 |

TABLE 32B

| Study Day | Task |
|---|---|
| Day 0 | Bodyweight (BW) measurements baseline. |
| | Paw thickness and Arthritis clinical score baseline. |
| | IP dosing of dexamethasone. |
| | Hotplate baseline |
| | TI (Test Items), Vehicle and dexamethasone IA dosing. |
| Day 1 | IP dosing of dexamethasone. |
| | IV mAb injection. |
| Day 2 | IP dosing of dexamethasone. |
| Day 3 | Body weight (BW) measurements. |
| | Paw thickness and Arthritis clinical score. |
| | IP dosing of dexamethasone. |
| | TI, Vehicle and dexamethasone IA dosing. |
| Day 4 | Body weight (BW) measurements. |
| | Paw thickness and Arthritis clinical score. |
| | IP dosing of dexamethasone. |
| | IP LPS injection. |
| Day 5 | Body weight (BW) measurements. |
| | Paw thickness and Arthritis clinical score. |
| | IP dosing of dexamethasone. |
| | Hot plate measurement. |
| Day 6 | Body weight (BW) measurements. |
| | Paw thickness and Arthritis clinical score. |
| | IP dosing of dexamethasone. |
| | TI, Vehicle and dexamethasone.IA dosing. |
| Day 7 | Body weight (BW) measurements. |
| | Paw thickness and Arthritis clinical score. |
| | IP dosing of dexamethasone. |
| Day 8 | Body weight (BW) measurements. |
| | Paw thickness and Arthritis clinical score. |
| | IP dosing of dexamethasone. |
| Day 9 | IP dosing of dexamethasone. |
| | TI, Vehicle and dexamethasone IA dosing. |
| Day 10 | Body weight (BW) measurements. |
| | Paw thickness and Arthritis clinical score. |
| | IP dosing of dexamethasone. |
| Day 11 | IP dosing of dexamethasone. |
| | Hot plate measurement. |
| Day 12 | Body weight (BW) measurements. |
| | Paw thickness and Arthritis clinical score. |
| | Study Termination-paws harvesting. |

Animals are examined for signs of arthritis on study day 0 for all paws and again on study days 3-8, 10 and 12 (front left and right paws, hind left and right paws). The signs of arthritis on study day 0 serve as a baseline for the arthritis clinical score parameter. Arthritis reactions are reported for each paw according to a 0-4 scale in ascending order of severity as shown below.

TABLE 33

| Arthritis Score | Grade |
|---|---|
| No reaction, normal | 0 |
| Mild, but definite redness and swelling of the | 1 |
| ankle/wrist or apparent redness and swelling | |
| limited to individual digits, regardless | |
| of the number of affected digits. | |
| Moderate to severe redness and swelling of the ankle/wrist. | 2 |
| Redness and swelling of the entire paw including digits. | 3 |
| Maximally inflamed limb with involvement of multiple joints. | 4 |

Paw thickness for both hind paws is measured on study day 0 and again on study days 3-8, 10 and 12. The measurements of paw thickness on study day 0 serve as the baseline for the paw volume parameter. The measurements are carried out using a digital caliper.

Throughout the 13-day study general clinical signs and observation are performed and recorded if any unexpected abnormalities are observed. These clinical signs are observed in addition to the ACAIA clinical scoring and assessment.

Individual body weights are determined on study day 0 and shortly before arthritis induction on study day 0. The weights measured on study day 0 are used as baseline measurements. From disease induction the animals are weighed daily on study days 3-8, 10 and 12 (study termination).

On days 0, 5 and 11 animals are placed on hot plate apparatus (53±1° C.) and the time until the first response is observed and recorded.

Animals found in a moribund condition and animals showing severe pain and enduring signs of severe distress (Score of 12 or more. Weight loss of 20%) are humanely euthanized.

At the end of the study, the mice are euthanized using pentobarbital sodium injection. Once euthanized, one hind paw of each animal is harvested and collected into vials containing 4% formalin for histology analysis. Each sample is labeled with the group number and animal number.

The samples are sagittal sections of the right and left murine tarsus. The histology scoring is based on the severity of the lesions, taking into account both the number of infiltrating cells and the extent of cartilage and bone damage. To be classified as severe, a lesion has to show bone and cartilage damage (beyond degeneration for the latter). The scores are 0=within normal limits, 1-minimal, 2-mild, 3-moderate, 4-severe.

Statistics and data evaluation are primarily based on the mean values for body weight, arthritis clinical scoring and paw thickness measurements. Where appropriate, analysis of the data by one-way ANOVA followed by Dunnett's post-test is applied to determine the significance of treatment effects. All data for the histopathological analysis is presented as Mean±SEM. The treatment groups are compared to the Vehicle group (Group 1) or the contralateral hind paw using the Student's T-test. A p-value <0.05 is considered to represent a significant difference.

As described in Example 8 below, the solubility of BETi was determined. The solubility in water was determined to be about 0.005 mg/gram or 0.5 mg/100 ml water (1 ml=1 g) using HPLC.

### Example 5. BETi1 Formulations Compared to Vehicle and Steroid in a Murine Anti Collagen Antibodies Induced Arthritis (ACAIA) Mimicking Rheumatoid Arthritis

Formulations were prepared in accordance with Experimental Method N. Investigation of the effect of different suspensions comprising four concentrations of BETi1 compared to vehicle and steroid controls in ACAIA mice was undertaken in accordance with the protocol set out in detail in Experimental Method O.

Inflammatory arthritis was induced in BALB/C mice using a mixture of four arthritogenic MAbs by iv at Day 0 and was further challenged with an LPS iv injection at Day 4 (N=7/treatment group). Vehicle (group 1) BETi1 treatment groups (groups 2-5) received 50p intra-articular (IA) doses of BETi1 at 0, 0.01, 0.1, 1 or 10 mg/kg on Days 0, 3, 6 and 9. Dexamethasone positive control animals (group 7) received 50 μl of 1 mg/kg IA on Days 0, 3, 6 and 9 and negative control (group 6) received 1 mg/kg IP on each treatment day (Day 0-11).

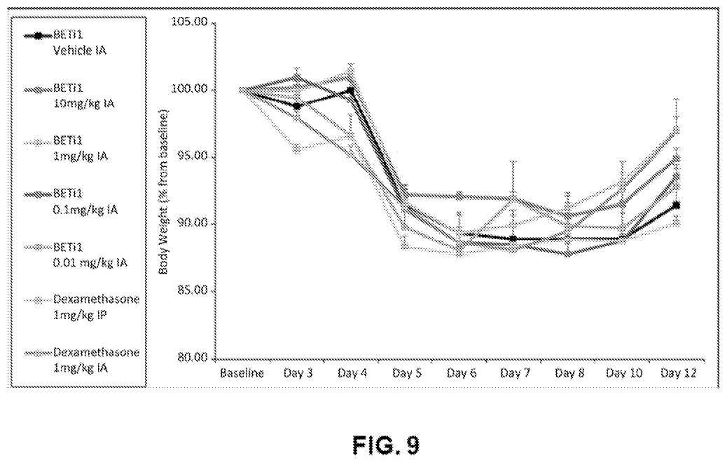

BETi1 demonstrated statistically significant improvement in treatment response for both endpoints at 1 mg/kg and 10 mg/kg doses. BETi1 also demonstrated numerically superior results at 1 mg/kg and 10 mg/kg doses compared to a leading injectable steroid, dexamethasone, in inhibiting inflammation in arthritic joints of mice. A demonstrated improvement in signs and symptoms of joint inflammation were observed in the limbs of animals treated with BETi1, whilst no treatment effect was observed in untreated limbs, suggesting a locally acting anti-inflammatory effect of BETi1. In contrast, animals that received systemic dexamethasone experienced a treatment effect in all limbs. Treatment tolerability was evaluated based on changes in body weight. Animals treated with systemic dexamethasone experienced continued weight loss throughout the study while all other treatment groups experienced improved body weights following recovery from the RA stimulant. As shown in Tables 34A and 34B and FIG. 9 following RA induction from day 4 to 6, all study groups animals had a clear reduction in body weight compared to baseline. Body weight loss plateaued and reversed for all BETi1 treatment groups at study day 5/6, on day 6/10 for animals receiving vehicle and on day 6/7 for animals receiving intra-articular steroid (Group 7). At the end of the study animals treated with intra-articular steroid (IA) and BETi1 gained more weight compared to vehicle. Animals treated with higher doses of BETi1 gained more weight in comparison animals treated with lower BETi1 doses. Weight gain at day 12 for highest BETi dose was similar to that of IA steroid. Weight loss for Group 3 (BETi1 1 mg/kg IA) was statistically significant compared to vehicle on day 12 (p<0.05). At day 12 all animals had lower body weights compared to baseline.

Group 6 (IP) which treated with steroids systemically (negative control) failed to experience meaningful weight gain. Weight loss for Group 6 was statistically significant compared to vehicle on day 3 (p<0.01). This was expected as weight loss is a common side effect of systemic steroids.

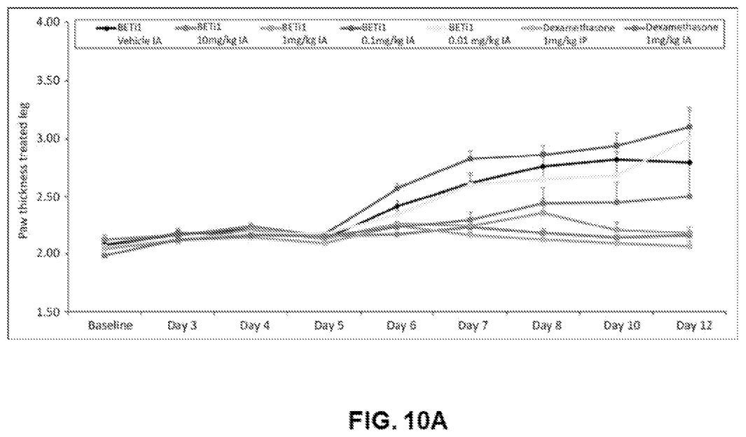

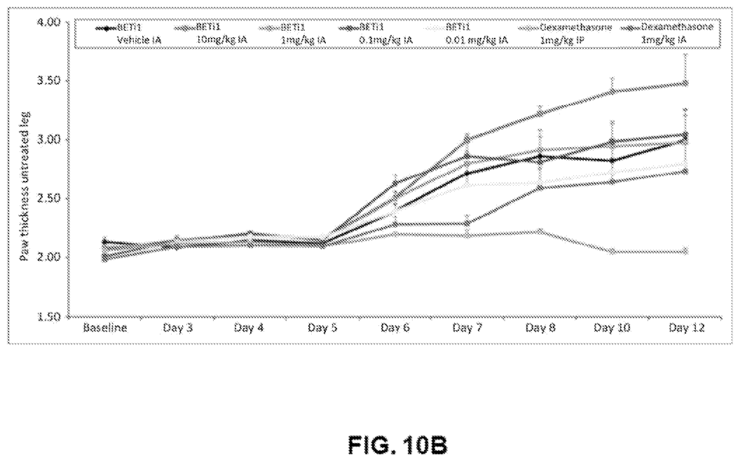

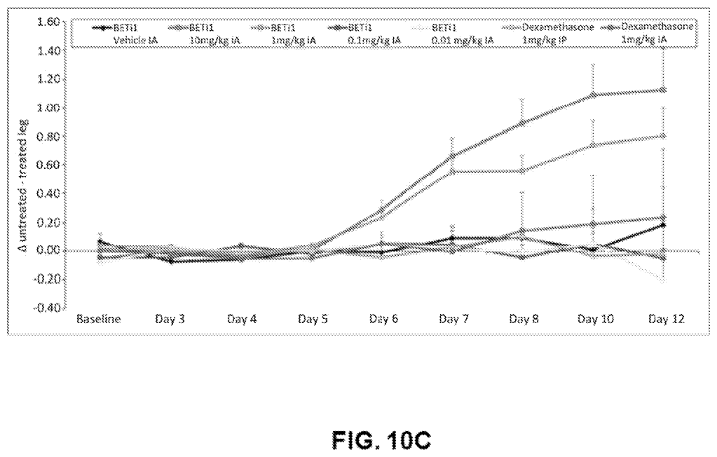

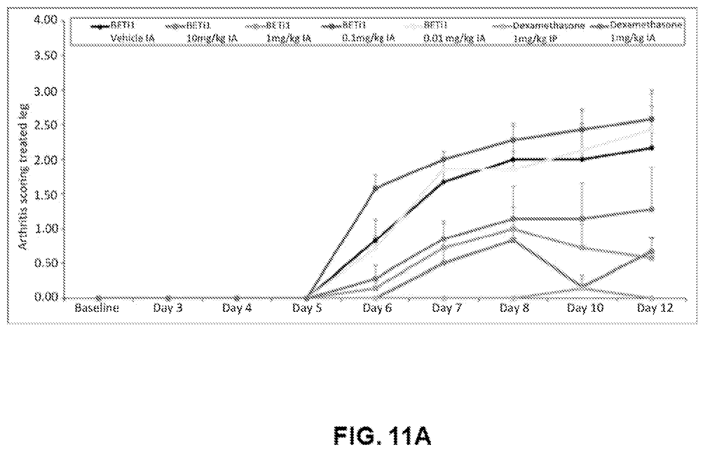

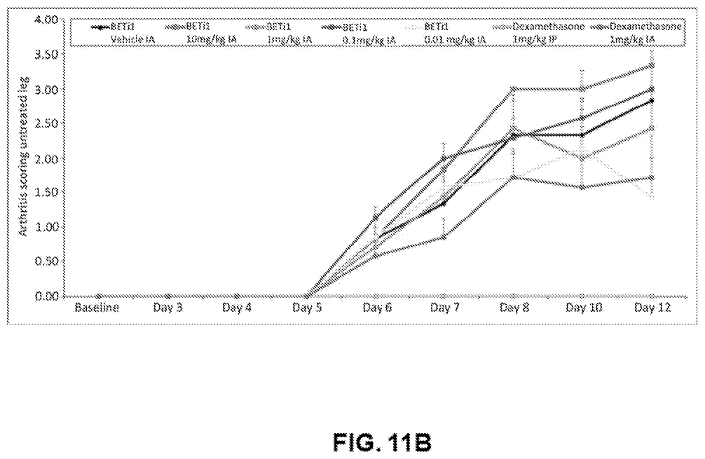

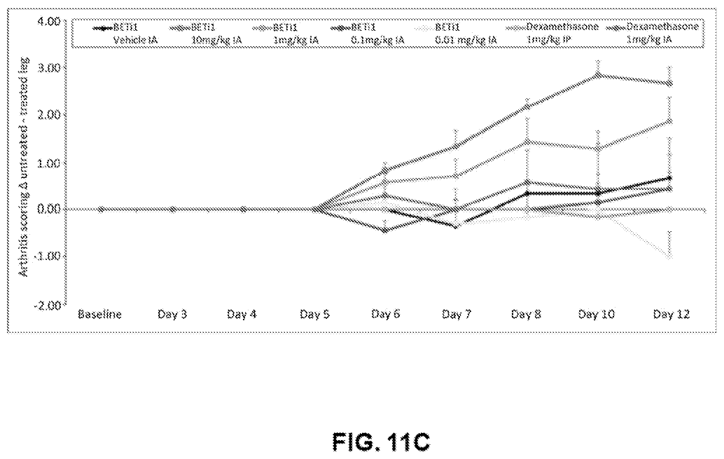

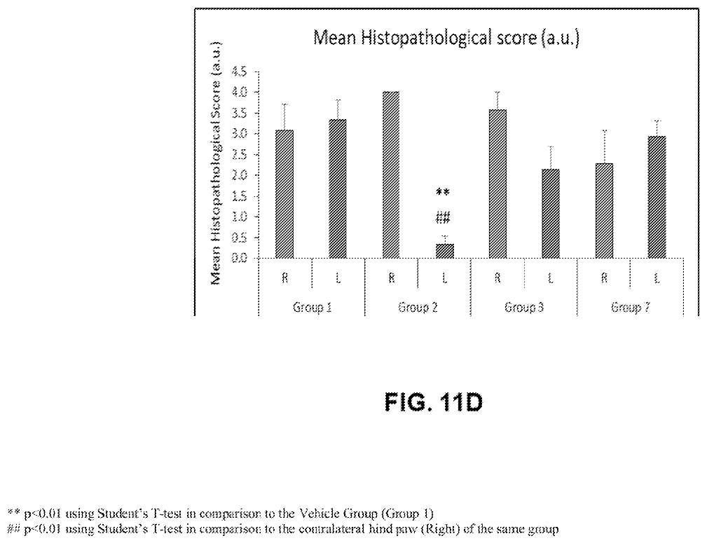

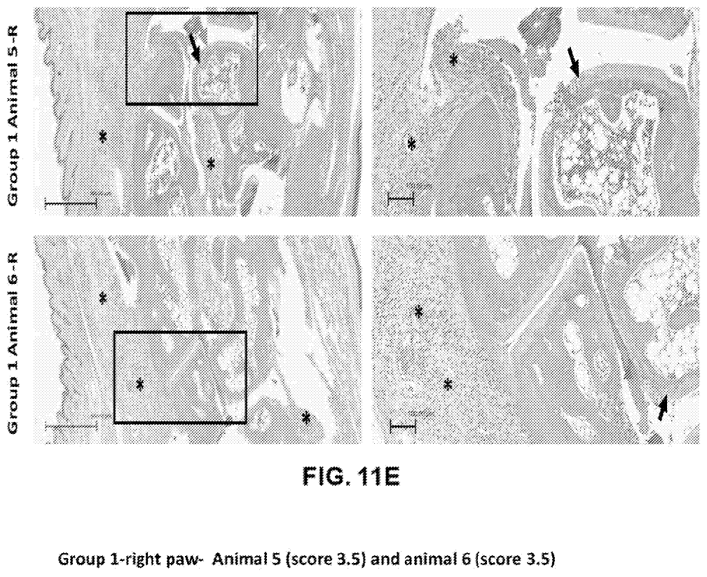

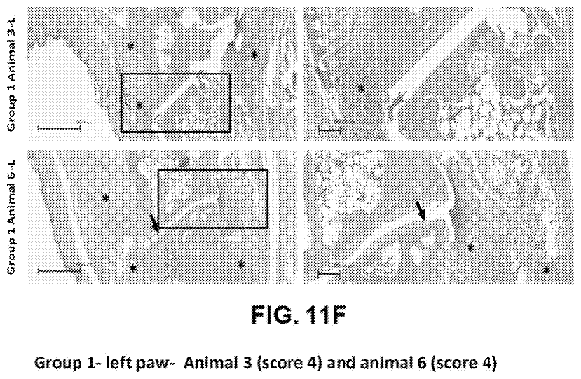

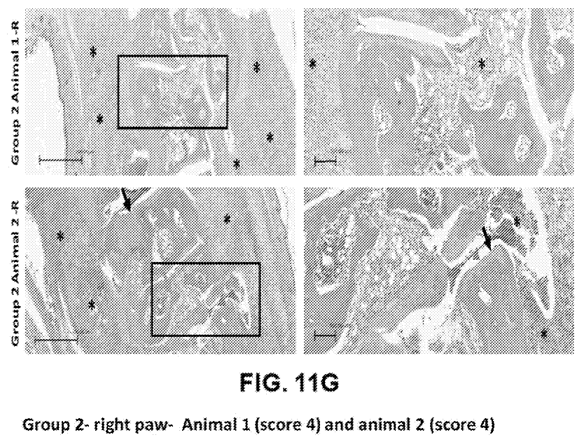

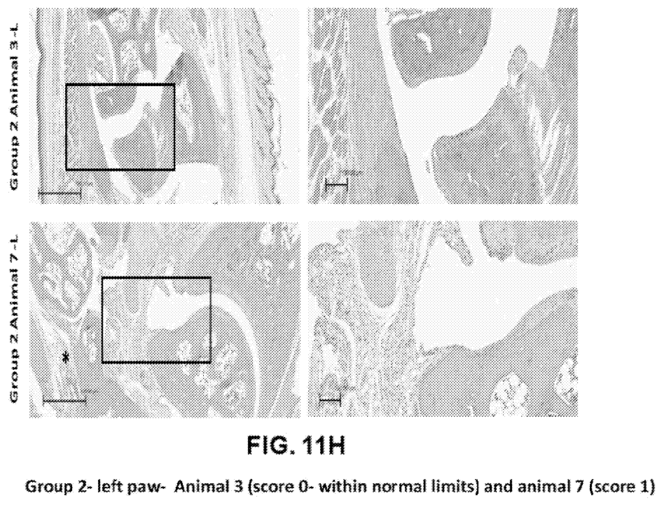

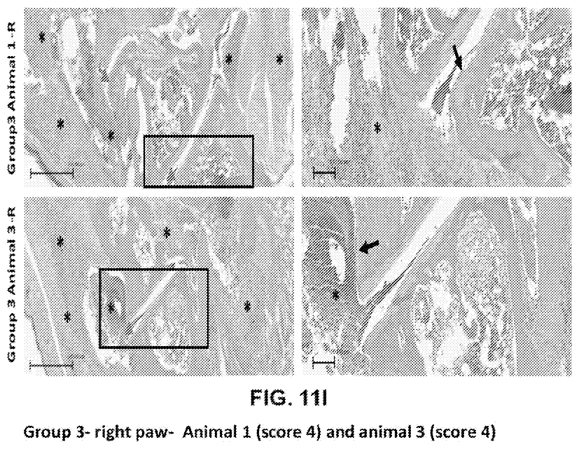

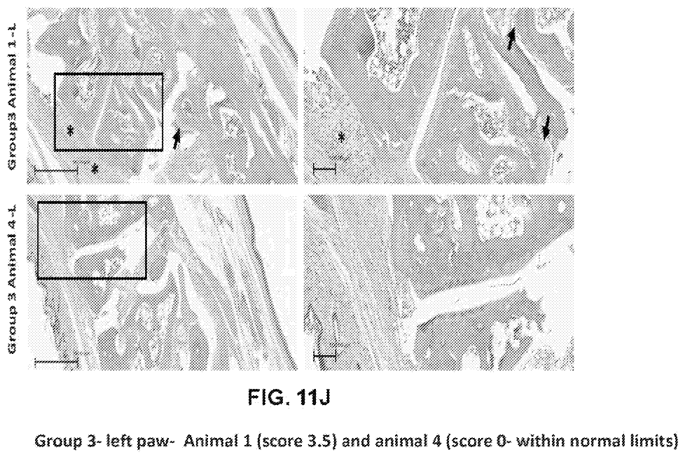

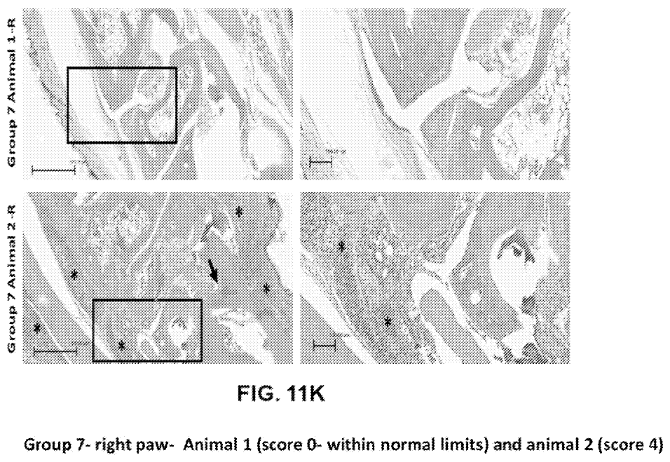

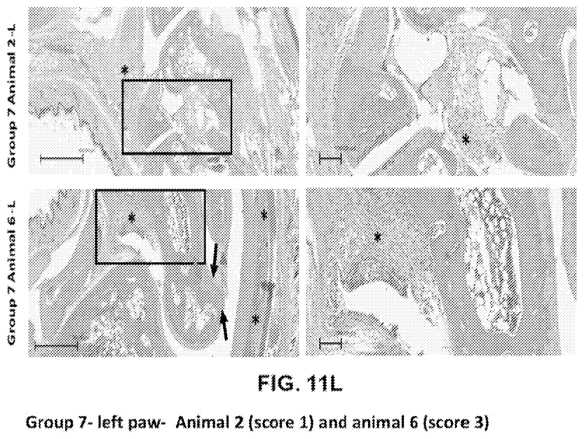

Surprisingly BETi1 at the two highest doses gave an excellent response and was superior to intra-articular steroid as evidenced by comparison of treated and untreated paw thickness (Table 35A-D and FIG. 10C) and arthritis scoring (Table 36A-D and FIG. 11C). BETi1, a pan-BET inhibitor, administered via intra-articular injection resulted in significant inhibition of inflammation. It should be noted that IP administration of the steroid serves as a negative control as it likely prevents the induction of the disease due to its systemic effects.

Paw thickening commenced at study days 5/6. No appreciable impact in prevention of paw thickening for vehicle and the two lowest doses of BETi1 (0.01 & 0.1 mg/kg) was observed, whereas a marked inhibition of paw thickening for the BETi1 1 & 10 mg/kg dose levels was observed. Inhibition was statically significant for 10 mg/kg BETi1 IA (Group 2) compared to vehicle on day 6 (p<0.001) and days 7, 8, 10 (p<0.01). Inhibition was also statically significant for 1 mg/kg BETi1 IA (Group 3) compared to vehicle on days 6, 8, 10 (p<0.05) and day 7 (p<0.01). In addition, inhibition was statically significant for 1 mg/kg IA steroid (Group 7) compared to vehicle on days 6 and 7 (p<0.01). Inhibition was also statically significant for 1 mg/kg IP steroid (Group 6) compared to vehicle on days 6, 7, 8, 10 and 12, however the IP administration of the steroid is probably not a fair comparison since it likely prevents the induction of the disease due to its systemic effects. Surprisingly, both BETi1 1 & 10 mg/kg were superior to dexamethasone IA 1 mg/kg and in line with 1 mg/kg dexamethasone systemic dose. Paw thickness was statically significant for 0.1 mg/kg IA (Group 4) compared to vehicle on day 6 (p<0.05) (Table 35A FIG. 10A). This thickening for this dose was only observed on day 6.

Table 35C and FIG. 10C show the delta between paw thickness of untreated (Table 35B FIG. 10B) versus treated leg where a higher delta indicates a better local treatment of joint inflammation. BETi1 1 & 10 mg/kg treatment groups demonstrated the strongest effect of all treatments for inhibiting paw thickening when compared to untreated paws. Treatment effect for BETi1 treated animals was dose-dependent over the dose range. The delta of inhibition was statically significant higher for 10 mg/kg BETi1 IA (Group 2) compared to vehicle on days 6, 7, 8 and 10 (p<0.001). The delta of inhibition was also statically significant higher for 1 mg/kg BETi1 IA (Group 3) compared to vehicle on days 6 and 7 (p<0.05). Paw thickness was also statically significant for 0.1 mg/kg IA (Group 4) compared to vehicle on day 4 (p<0.05). However, day 4 is prior to full induction of the disease so on the face of it is not meaningful.

Without being bound by any theory, while vehicle-treated, IP steroid mice, and the two low BETi1 doses developed active arthritis as evidenced by low delta of paw thickness at day 12, administration of BETi ameliorated joint inflammation in a dose-dependent manner (i.e. the highest dose 10 mg/kg demonstrated the highest delta paw thickness).

Additional statistical comparisons were made and provided in Table 35D and Table 35E using a T-test (paired, 2 tails, alpha=0.05). As shown in Table 35D treated paws were compared to untreated per group for each day. The statistical comparison shows that the two highest BETi1 doses (at 10 mg/kg and 1 mg/kg) were consistently significant from day 6 through day 12. At both highest doses, the inhibition of paw thickening was statistically significant in the treated paw relative to the untreated rear paw on day 12 (p<0.01). For these BETi1 treatment groups, mean paw thickness at baseline (day 0) was about 2.08 mm. For the 10 mg/kg dose, the average paw thickness at day 12 was about 3.48 mm in the untreated paw versus 2.17 mm in the treated paw, representing a 37.6% reduction. For the 1 mg/kg dose, the average paw thickness at day 12 was about 2.98 mm in the untreated paw versus about 2.18 mm in the treated paw, representing a 26.8% reduction. Results with these two doses were numerically superior to intra-articular dexamethasone and consistent with the systemic dexamethasone treatment.

The lack of significance in Dexamethasone 1 mg/kg IP on days 10 and 12 was likely due to the similar efficacy on both paws (systemic distribution of the steroid).

As shown in Table 35E paw thickness on Day 12 was compared to baseline. It was observed that the two highest BETi1 doses (at 10 mg/kg and 1 mg/kg), as well as Dexamethasone 1 mg/kg IP and IA were not statistically significant (meaning paws at D12 as “healthy” as at DO).

The arthritis score results were consistent with the paw thickness results. Arthritis severity was quantifiable at study days 5/6. No appreciable impact in arthritis severity for vehicle and the two lowest doses of BETi1 (0.01 & 0.1 mg/kg). Marked inhibition of arthritis signs and symptoms was observed for BETi1 1 & 10 mg/kg dose levels. Surprisingly, both BETi1 1 & 10 mg/kg were superior to dexamethasone IA 1 mg/kg. The score was statically significant lower for 10 mg/kg BETi1 IA (Group 2) compared to vehicle on days 6, 7 and 10 (p<0.05). The score was also statically significant lower for 1 mg/kg IP steroid (Group 6) compared to vehicle on days 6, 7, 8, 10 and 12, however the IP administration of the steroid is probably not a fair comparison since it likely prevents the induction of the disease due to its systemic effects. As observed above for paw thickness the score was also statically significant higher for 0.1 mg/kg IA (Group 4) compared to vehicle only on day 6 (p<0.01). (FIG. 11B).

BETi1 1 & 10 mg/kg treatment groups demonstrated the strongest effect of all treatments on arthritis severity scores when compared to untreated limbs. Treatment effect for BETi1 treated animals was dose-dependent over the dose range. The score delta was statically significant higher for 10 mg/kg BETi1 IA (Group 2) compared to vehicle on days 6, 7, 8 and 10 (p<0.05) (FIG. 11C).

Additional statistical comparisons were made and provided in Table 36D and Table 36E using a T-test (paired, 2 tails, alpha=0.05). As shown in Table 36D the clinical score of treated paws were compared to untreated paws per group for each day. The statistical comparison shows that the two highest BETi1 doses (at 10 mg/kg and 1 mg/kg) were consistently significant from day 8 through day 12. The lack of significance in Dexamethasone 1 mg/kg IP was likely due to the similar efficacy on both paws (systemic distribution of the steroid). Limbs treated with BETi1 at the 1 and 10 mg/kg dose levels had an average arthritis score of about 0.57 and about 0.67, respectively, or near normal. The arthritis score was significantly lower in the treated paw at both doses relative to the untreated paws on day 12 (p<0.05). At the 10 mg/kg dose, the average arthritis score was about 0.67 in the treated paw versus about 3.33 in the untreated paw, representing a 79.9% reduction. At the 1 mg/kg dose, the average arthritis score was about 0.57 in the treated paw versus about 2.43 in the untreated paw, representing a 76.5% reduction. Limbs treated with intra-articular dexamethasone received an average arthritis score of 1.3, indicating mild symptoms. These animals had signs of redness and swelling of the ankle/wrist or apparent redness and swelling limited to individual digits.

As shown in Table 36E average clinical score of each group on Day 12 was compared to baseline. It was observed that the BETi1 at 1 mg/kg, as well as Dexamethasone 1 mg/kg IP and IA were not statistically significant (meaning clinical score at D12 was comparable to DO). The data for BETi1 10 mg/kg IA was statistically significant in this case meaning it was not comparable.

Histopathological analysis was conducted in accordance with Method B. Tissues from groups 1, 2, 3 and 7 were processed. Following decalcification, the tissues were cut mid-sagitally and were processed and sectioned for H & E staining. The histopathological analysis included an examination of the number of infiltrating cells and the extent of cartilage and bone damage.

In general, cases classified as severe had inflammation with bone and cartilage damage in the tarsus and metatarso-phalangeal joints (when present). However, consistent standardization was difficult because some cases had very severe inflammatory infiltration with relatively limited bone and cartilage damage whilst others had moderate inflammation with significant bone and cartilage damage. Both these lesion patterns were classified as severe in this study. Additionally, in some cases (6/52) there was unusual band of slightly disorganized hyaline cartilage along the outer aspect of either tibia (1/6), tarsal (4/6) or metatarsal (2/6) bones. The location indicates of periosteal origin. Suggesting subperiosteal cartilage and bone proliferation produced by the lifted periosteum. This is an unusual lesion, which without further information remains unexplained.

In limbs with lesions, the most common score was severe and consisted of chronic-active inflammatory infiltration, varying in the proportion of mononuclear cells and neutrophils, and involving soft tissues, joint capsule, bones (tarsal, metatarsal and phalangeal, according to the sample), joint space and articular surfaces.

In Group 1 in 4/6 animals similar severity grades were found for both hind limbs. All four animals presented severe inflammation, but the severity of the lesions involving the cartilage and bone differed (two were graded severe and two were graded moderate-severe). One animal presented severe lesions on its left hind limb while moderate to severe on the right hind limb. One animal was WNL on its right and had minimal inflammation on the left.

In 3/6 cases treated with vehicle (Group 1L) there were severe lesions with destruction of cartilage and bone. Two cases show severe inflammation with moderate to severe damage to bone and cartilage. One case was classified as minimal with presence of minimal inflammation in the soft tissue. (FIG. 11F) In 2/6 cases of untreated paws in the same group (Group 1R) there were severe lesions with destruction of cartilage and bone. Three cases show severe inflammation with moderate to severe damage to bone and cartilage. One case was within normal limits (WNL) (FIG. 11E).

All the animals in group 2 (10 mg/kg BETi1) showed differences between the right hind limb (severely affected) and the left hind limb (minimally affected or WNL). Three animals (#1, 5 and 7) present subperiosteal cartilage and bone proliferation, all were on the right hind limb.

In 2/6 cases treated with 10 mg/kg BETi1 (group 2L) there was minimal inflammation without damage to cartilage and bone, while 4/6 were WNL (FIG. 11H). Whereas all cases of untreated paws in the same group (group 2R) had severe inflammation and severe lesions with destruction of cartilage and bone. 3/3 cases presented subperiosteal cartilage and bone proliferation on one of the tarsal bones (#1, 5 and 7), one limb presented subperiosteal cartilage and bone proliferation also on the distal tibia (#7). (FIG. 11G)

In group 3 (1 mg/kg BETi1), all the animals showed differences between the treated and untreated hind limbs. 3/7 animals presented severe lesions at the right hind limb level while the left presented moderate to severe lesions. 2/7 animals presented severe lesions at the right hind limb level while the left presented minimal inflammation. One animal presented severe lesions at the right hind limb level while the left was WNL. One animal presented minimal inflammation at the right hind limb level while the left hind limb presented mild to moderate lesions. One animal (#7) presented subperiosteal cartilage and bone proliferation on the left hind limb.

In 3/7 cases treated with 1 mg/kg BETi1 (Group 3L) moderate to severe lesions with destruction of cartilage and bone were found. One case presented mild to moderate lesions with some destruction of cartilage and bone. Two cases presented minimal inflammation without damage to cartilage and bone. One case was WNL. One animal (#7) presented subperiosteal cartilage and bone proliferation on the distal metatarsus (FIG. 11J). Whereas, for 6/7 cases of untreated paws in the same group (Group 3R) there were severe lesions with destruction of cartilage and bone. One case presented a minimal inflammation in the soft tissue, without damage to cartilage and bone (FIG. 11I).

All the animals showed differences between the treated and untreated hind limbs. 2/7 animals presented severe lesions at the right hind limb level while the left presented moderate lesions. One animal presented severe lesions at the right hind limb level while the left presented mild to moderate lesions. One animal presented severe lesions at the right hind limb level while the left presented minimal inflammation. In two animals the right hind limb was WNL while the left presented severe lesions. In one animal the right hind limb is WNL while the left presented moderate lesions. Two animals (#3 and 4) presented subperiosteal cartilage and bone proliferation on the left hind limb.

In 2/7 cases treated with dexamethasone 1 mg/kg IA (Group 7L) there were severe lesions with destruction of cartilage and bone. Three cases showed moderate lesions with destruction of cartilage and bone. One case presented mild to moderate lesions and another only minimal inflammation without damage to cartilage and bone. Two cases present subperiosteal cartilage and bone proliferation, case 3L on one of the tarsal bones, and case 4L on the distal metatarsus (FIG. 11L). Whereas in 4/7 cases of untreated paws in the same group (Group 7R) there were severe lesions with destruction of cartilage and bone. The other three were WNL (FIG. 11K).

The mean histopathological results of the vehicle, two highest doses and steroid controls (groups 1, 2, 3, 7) are shown in Table 36F and FIG. 11D. The treatment groups were compared to the vehicle group (Group 1) or the contralateral hind paw using the Student's T-test. A p-value <0.05 was considered to represent a significant difference.

FIG. 11D shows a histogram of the mean histopathological score (a.u.) of untreated paw (right paw) versus treated paw for vehicle steroid and two highest doses where a higher difference between the paws means better local treatment.

Statistical analysis showed a statistically significant reduction in the mean histopathological score following IA injection of BETi1 at a dose of 10 mg/kg (Group 2, left hind paw), in comparison to the contralateral hind paw (0.33±0.21 vs 4.00±0.00 for left vs. right hind paws, respectively, p<0.01) or to the Vehicle group (0.33±0.21 vs. 3.33±0.48 for left paws of Group 2 vs. left paws of Group 1, respectively, p<0.01). Treatment with BETi1 at a lower dose of 1 mg/kg (Group 3, left hind paw), showed a trend of reduction in the mean histopathological score (2.14±0.55 vs. 3.33±0.48 for lefts paws of Group 3 vs. Group 1, respectively), yet without statistical significance. Local treatment of dexamethasone (IA, Group 7) showed a trend of reduction, however, without statistical significance. Under the conditions of the study, a statistically significant reduction in the mean histopathological score was found following IA injection of BETi1 at a dose of 10 mg/kg (Group 2, left hind paw), in comparison to the contralateral hind paw or to the Vehicle group (Group 1), in an ACAIA mouse model.

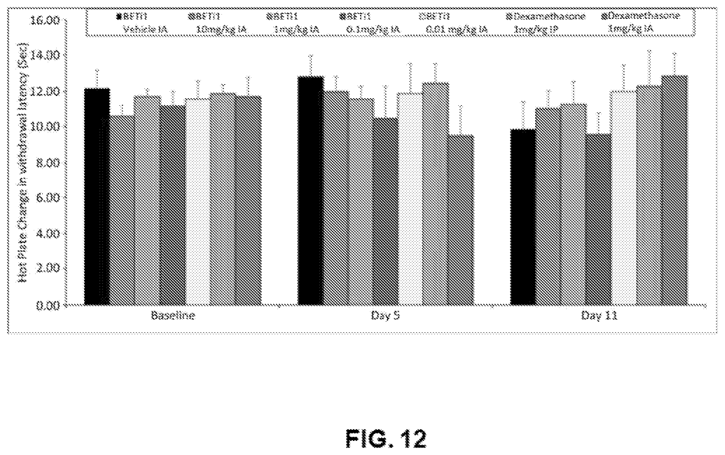

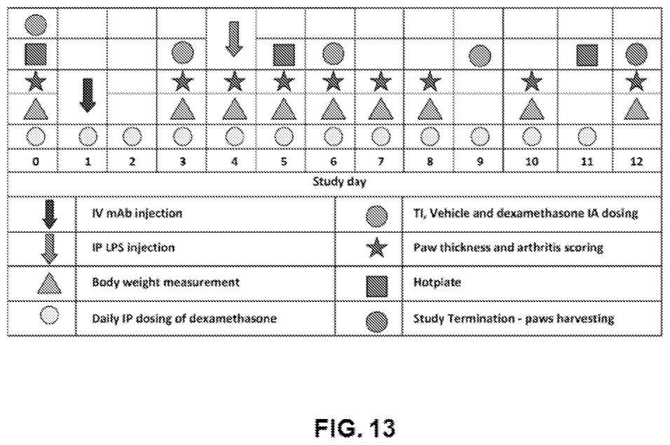

No conclusions could be clearly derived from hotplate change in withdrawal latency between baseline and days 5 and 11 (Table 37 and FIG. 12).

TABLE 34A
Weight per day for different treatments (mg)

| Treatment | | | | | | | | | | |
|---|---|---|---|---|---|---|---|---|---|---|
| Group/ | | | | | | | | | | |
| Weight | | | | | | | | | | |
| Per Day | Ani. # | Baseline | Day 3 | Day 4 | Day 5 | Day 6 | Day 7 | Day 8 | Day 10 | Day 12 |
| BETi1 | 1 | 22.2 | 22.2 | 22.3 | 20.8 | 20 | 19.6 | 19.7 | 19.7 | 20 |
| Vehicle IA | 2 | 21.6 | 21.9 | 22.2 | 20.2 | 19.9 | 19.8 | 20 | 19.7 | 20.4 |
| (Group 1) | 3 | 22.2 | 21 | 21.5 | 19.5 | 19 | 18.8 | 18.8 | 19 | 19.6 |
| | 4 | 21.5 | 20.9 | 21.2 | 20.1 | 20.4 | 20.7 | 20.7 | 20.9 | 21.2 |
| | 5 | 20.2 | 19.8 | 19.9 | 17.9 | 17.4 | 17.6 | 17.3 | 17.4 | 18.5 |
| | 6 | 20.9 | 20.7 | 20.9 | 19.2 | 18.3 | 17.9 | 17.9 | 17.7 | 17.9 |
| | 7 | 18.8 | 19 | 19.4 | | | | | | |
| | Av | 21.06 | 20.79 | 21.06 | 19.62 | 19.17 | 19.07 | 19.07 | 19.07 | 19.60 |
| | Sd | 0.46 | 0.42 | 0.41 | 0.41 | 0.47 | 0.49 | 0.53 | 0.54 | 0.50 |
| BETi1 | 1 | 20.2 | 20.1 | 20.2 | 18.5 | 18.9 | 18.7 | 18.6 | 18.3 | 19.3 |
| 10 mg/kg IA | 2 | 21.6 | 21.5 | 21.4 | 20.1 | 19.8 | 19.4 | 19.2 | 19.2 | 20.3 |
| (Group 2) | 3 | 20.8 | 21.3 | 21.4 | 19.3 | 19.2 | 19.5 | 19.6 | 19.8 | 20.4 |
| | 4 | 21.7 | 22 | 22.4 | | | | | | |
| | 5 | 21.4 | 21.5 | 21.6 | 20.3 | 19.5 | 19.6 | 18.8 | 19.6 | 19.9 |
| | 6 | 22.2 | 21.8 | 21.9 | 19.9 | 20.4 | 20.3 | 20.2 | 20.5 | 21.1 |
| | 7 | 21.3 | 21.3 | 21.7 | 19.4 | 19.5 | 19.6 | 19.1 | 19.3 | 20 |
| | Av | 21.31 | 21.36 | 21.51 | 19.58 | 19.55 | 19.52 | 19.25 | 19.45 | 20.17 |
| | Sd | 0.25 | 0.23 | 0.25 | 0.27 | 0.21 | 0.21 | 0.24 | 0.30 | 0.24 |
| BETi1 | 1 | 20.4 | 20.6 | 20.8 | 19.1 | 18.7 | 18.3 | 18.5 | 18.8 | 19.4 |
| 1 mg/kg IA | 2 | 22.5 | 22.1 | 22.5 | 20.2 | 18.6 | 18.9 | 19.8 | 21.3 | 22.5 |
| (Group 3) | 3 | 20.3 | 20.8 | 21.1 | 19.2 | 18.8 | 18.9 | 18.8 | 19 | 19.9 |
| | 4 | 19.9 | 19.6 | 19.9 | 17.7 | 18 | 17.8 | 17.9 | 18.6 | 19.3 |
| | 5 | 21.5 | 21 | 21.7 | 19.2 | 19.3 | 19.4 | 19.3 | 19.3 | 20.1 |
| | 6 | 19.7 | 19.8 | 19.9 | 17.9 | 17.2 | 17.7 | 18.2 | 18.7 | 19.4 |
| | 7 | 20.6 | 20.9 | 21 | 19.5 | 18.8 | 19.2 | 19.6 | 19.2 | 20.1 |
| | Av | 20.70 | 20.69 | 20.99 | 18.97 | 18.49 | 18.60 | 18.87 | 19.27 | 20.10 |
| | Sd | 0.37 | 0.31 | 0.35 | 0.33 | 0.26 | 0.25 | 0.27 | 0.35 | 0.42 |
| BETi1 | 1 | 20.2 | 20 | 20 | 18.1 | 17.3 | 17.8 | 17.5 | 18.2 | 18.8 |
| 0.1 mg/kg IA | 2 | 21 | 21.2 | 21.3 | 19 | 18.7 | 18.8 | 18.4 | 19 | 19.9 |
| (Group 4) | 3 | 19.7 | 20.3 | 18 | 16.6 | 16.5 | 16.8 | 16.7 | 16.8 | 17.5 |
| | 4 | 21.9 | 22.4 | 21.9 | 20.5 | 19.7 | 19.5 | 19.5 | 19.4 | 20.7 |
| | 5 | 20.3 | 20.6 | 20.4 | 19.4 | 18.5 | 18.2 | 18.4 | 17.7 | 18.7 |
| | 6 | 21.5 | 21.2 | 21.4 | 19.4 | 18.9 | 18.8 | 18.4 | 19.1 | 20.2 |
| | 7 | 18.5 | 18.8 | 19.1 | 17.4 | 17.2 | 16.7 | 16.6 | 16.8 | 18 |
| | Av | 20.44 | 20.64 | 20.30 | 18.63 | 18.11 | 18.09 | 17.93 | 18.14 | 19.11 |
| | Sd | 0.44 | 0.43 | 0.53 | 0.51 | 0.43 | 0.40 | 0.40 | 0.41 | 0.45 |
| BETi1 | 1 | 21 | 20.8 | 21.1 | 19.5 | 19.1 | 19.4 | 19.3 | 19.6 | 19.9 |
| 0.01 mg/kg IA | 2 | 20.9 | 20.6 | 20.8 | 18.7 | 19 | 19.3 | 19.7 | 19.5 | 20 |
| (Group 5) | 3 | 21.5 | 21.6 | 20.9 | 19.1 | 18.6 | 18.9 | 18.9 | 18.3 | 19.1 |
| | 4 | 21.4 | 20.9 | 21.6 | 20.2 | 19.9 | 20 | 20.1 | 19.5 | 20.03 |
| | 5 | 19.4 | 19.3 | 17.4 | 16.6 | 15.6 | 16 | 16 | 16.2 | 16.9 |
| | 6 | 18.7 | 18.8 | 17.8 | 16.8 | 16.5 | 19.9 | 16.9 | 17.4 | 18.2 |
| | 7 | 21 | 21 | 19.6 | 18.4 | 18.1 | 18.5 | 18.5 | 18.6 | 19.3 |
| | Av | 20.56 | 20.43 | 19.89 | 18.47 | 18.11 | 18.86 | 18.49 | 18.44 | 19.06 |
| | Sd | 0.41 | 0.38 | 0.63 | 0.51 | 0.58 | 0.52 | 0.57 | 0.48 | 0.44 |
| Dexamethasone | 1 | 19.9 | 18.9 | 19.2 | 17.6 | 17.6 | 17.8 | 17.9 | 17.8 | 18.3 |
| 1 mg/kg IP | 2 | 20.3 | 19.1 | 19.4 | 17.5 | 17.6 | 17.3 | 17.3 | 17.6 | 17.9 |
| (Group 6) | 3 | 20.3 | 19.3 | 19.7 | 18.6 | 18.2 | 18 | 17.8 | 17.9 | 18.5 |
| | 4 | 21.7 | 20.9 | 20.9 | 18.7 | 18.7 | 19 | 19.8 | 19.8 | 19.7 |
| | 5 | 21.3 | 20.5 | 20.6 | 18.5 | 18.5 | 18.9 | 18.9 | 18.8 | 19.2 |
| | 6 | 19.5 | 18.8 | 19 | 17.5 | 17.5 | 17.6 | 17.8 | 17.4 | 17.4 |
| | 7 | 20.6 | 19.8 | 19.9 | 18.4 | 17.8 | 18.3 | 18.1 | 18.2 | 18.4 |
| | Av | 20.51 | 19.61 | 19.81 | 18.11 | 17.99 | 18.13 | 18.23 | 18.21 | 18.49 |
| | Sd | 0.29 | 0.31 | 0.27 | 0.21 | 0.18 | 0.24 | 0.32 | 0.32 | 0.29 |
| Dexamethasone | 1 | 19.1 | 19.1 | 18.7 | 17.3 | 16.6 | 17 | 17.2 | 17.4 | 17.8 |
| 1 mg/kg IA | 2 | 20.3 | 19.8 | 19.6 | 18.5 | 17.8 | 16.9 | 16.9 | 18.8 | 20.1 |
| (Group 7) | 3 | 18.6 | 17.8 | 17.3 | 16.5 | 16 | 16 | 16.3 | 16.3 | 16.8 |
| | 4 | 19.2 | 19.1 | 17.9 | 19.2 | 19.6 | 20 | 20.1 | 20 | 21.2 |
| | 5 | 20.2 | 19.9 | 19.2 | 18.2 | 17.7 | 17.4 | 18 | 18.2 | 19.1 |
| | 6 | 20.2 | 19.7 | 19.1 | 17.7 | 17.1 | 15.8 | 16.4 | 18.2 | 19.1 |
| | 7 | 20.9 | 20.6 | 20.1 | 19 | 17.9 | 18.7 | 18.9 | 19.4 | 20.1 |
| | Av | 19.79 | 19.48 | 18.84 | 18.06 | 17.53 | 17.40 | 17.69 | 18.33 | 19.17 |
| | Sd | 0.31 | 0.39 | 0.37 | 0.36 | 0.43 | 0.56 | 0.53 | 0.47 | 0.56 |

TABLE 34B
Percent change of weight per day for different treatments

| Treatment | | | | | | | | | | |
|---|---|---|---|---|---|---|---|---|---|---|
| Group/ | | | | | | | | | | |
| Weight | | | | | | | | | | |
| Per Day | Ani. # | Baseline | Day 3 | Day 4 | Day 5 | Day 6 | Day 7 | Day 8 | Day 10 | Day 12 |
| BETi1 | 1 | 100 | 100 | 100.4505 | 93.69369 | 90.09009 | 88.28829 | 88.73874 | 88.73874 | 90.09009 |
| Vehicle IA | 2 | 100 | 101.3889 | 102.7778 | 93.51852 | 92.12963 | 91.66667 | 92.59259 | 91.2037 | 94.44444 |
| (Group 1) | 3 | 100 | 94.59459 | 96.84685 | 87.83784 | 85.58559 | 84.68468 | 84.68468 | 85.58559 | 88.28829 |
| | 4 | 100 | 97.2093 | 98.60465 | 93.48837 | 94.88372 | 96.27907 | 96.27907 | 97.2093 | 98.60465 |
| | 5 | 100 | 98.0198 | 98.51485 | 88.61386 | 86.13861 | 87.12871 | 85.64356 | 86.13861 | 91.58416 |
| | 6 | 100 | 99.04306 | 100 | 91.86603 | 87.55981 | 85.64593 | 85.64593 | 84.689 | 85.64593 |
| | 7 | 100 | 101.0638 | 103.1915 | | | | | | |
| | Av | 100.00 | 98.76 | 100.06 | 91.50 | 89.40 | 88.95 | 88.93 | 88.93 | 91.44 |
| | Sd | 0.00 | 0.90 | 0.88 | 1.08 | 1.49 | 1.77 | 1.89 | 1.92 | 1.88 |
| BETi1 | 1 | 100 | 99.50495 | 100 | 91.58416 | 93.56436 | 92.57426 | 92.07921 | 90.59406 | 95.54455 |
| 10 mg/kg IA | 2 | 100 | 99.53704 | 99.07407 | 93.05556 | 91.66667 | 89.81481 | 88.88889 | 88.88889 | 93.98148 |
| (Group 2) | 3 | 100 | 102.4038 | 102.8846 | 92.78846 | 92.30769 | 93.75 | 94.23077 | 95.19231 | 98.07692 |
| | 4 | 100 | 101.3825 | 103.2258 | | | | | | |
| | 5 | 100 | 100.4673 | 100.9346 | 94.85981 | 91.1215 | 91.58879 | 87.85047 | 91.58879 | 92.99065 |
| | 6 | 100 | 98.1982 | 98.64865 | 89.63964 | 91.89189 | 91.44144 | 90.99099 | 92.34234 | 95.04505 |
| | 7 | 100 | 100 | 101.8779 | 91.07981 | 91.5493 | 92.01878 | 89.67136 | 90.61033 | 93.89671 |
| | Av | 100.00 | 100.21 | 100.95 | 92.17 | 92.02 | 91.86 | 90.62 | 91.54 | 94.92 |
| | Sd | 0.00 | 0.52 | 0.68 | 0.74 | 0.35 | 0.53 | 0.95 | 0.87 | 0.73 |
| BETi1 | 1 | 100 | 100.9804 | 101.9608 | 93.62745 | 91.66667 | 89.70588 | 90.68627 | 92.15686 | 95.09804 |
| 1 mg/kg IA | 2 | 100 | 98.22222 | 100 | 89.77778 | 82.66667 | 84 | 88 | 94.66667 | 100 |
| (Group 3) | 3 | 100 | 102.4631 | 103.9409 | 94.58128 | 92.61084 | 93.10345 | 92.61084 | 93.59606 | 98.02956 |
| | 4 | 100 | 98.49246 | 100 | 88.94472 | 90.45226 | 89.44724 | 89.94975 | 93.46734 | 96.98492 |
| | 5 | 100 | 97.67442 | 100.9302 | 89.30233 | 89.76744 | 90.23256 | 89.76744 | 89.76744 | 93.48837 |
| | 6 | 100 | 100.5076 | 101.0152 | 90.86294 | 87.30964 | 89.84772 | 92.38579 | 94.92386 | 98.47716 |
| | 7 | 100 | 101.4563 | 101.9417 | 94.66019 | 91.26214 | 93.20388 | 95.14563 | 93.20388 | 97.57282 |
| | Av | 100.00 | 99.97 | 101.40 | 91.68 | 89.39 | 89.93 | 91.22 | 93.11 | 97.09* |
| | Sd | 0.00 | 0.69 | 0.52 | 0.96 | 1.29 | 1.16 | 0.89 | 0.66 | 0.82 |
| BETi1 | 1 | 100 | 99.0099 | 99.0099 | 89.60396 | 85.64356 | 88.11881 | 86.63366 | 90.09901 | 93.06931 |
| 0.1 mg/kg IA | 2 | 100 | 100.9524 | 101.4286 | 90.47619 | 89.04762 | 89.52381 | 87.61905 | 90.47619 | 94.7619 |
| (Group 4) | 3 | 100 | 103.0457 | 91.37056 | 84.26396 | 83.75635 | 85.27919 | 84.77157 | 85.27919 | 88.83249 |
| | 4 | 100 | 102.2831 | 100 | 93.60731 | 89.95434 | 89.0411 | 89.0411 | 88.58447 | 94.52055 |
| | 5 | 100 | 101.4778 | 100.4926 | 95.5665 | 91.133 | 89.65517 | 90.64039 | 87.19212 | 92.11823 |
| | 6 | 100 | 98.60465 | 99.53488 | 90.23256 | 87.90698 | 87.44186 | 85.5814 | 88.83721 | 93.95349 |
| | 7 | 100 | 101.6216 | 103.2432 | 94.05405 | 92.97297 | 90.27027 | 89.72973 | 90.81081 | 97.2973 |
| | Av | 100.00 | 101.00 | 99.30 | 91.11 | 88.63 | 88.48 | 87.72 | 88.75 | 93.51 |
| | Sd | 0.00 | 0.62 | 1.42 | 1.42 | 1.20 | 0.64 | 0.83 | 0.75 | 0.99 |
| BETi1 | 1 | 100 | 99.04762 | 100.4762 | 92.85714 | 90.95238 | 92.38095 | 91.90476 | 93.33333 | 94.7619 |
| 0.01 mg/kg IA | 2 | 100 | 98.56459 | 99.52153 | 89.47368 | 90.90909 | 92.3445 | 94.25837 | 93.30144 | 95.69378 |
| (Group 5) | 3 | 100 | 100.4651 | 97.2093 | 88.83721 | 86.51163 | 87.90698 | 87.90698 | 85.11628 | 88.83721 |
| | 4 | 100 | 97.66355 | 100.9346 | 94.39252 | 92.99065 | 93.45794 | 93.92523 | 91.1215 | 93.59813 |
| | 5 | 100 | 99.48454 | 89.69072 | 85.56701 | 80.41237 | 82.47423 | 82.47423 | 83.50515 | 87.1134 |
| | 6 | 100 | 100.5348 | 95.18717 | 89.83957 | 88.23529 | 106.4171 | 90.37433 | 93.04813 | 97.3262 |
| | 7 | 100 | 100 | 93.33333 | 87.61905 | 86.19048 | 88.09524 | 88.09524 | 88.57143 | 91.90476 |
| | Av | 100.00 | 99.39 | 96.62 | 89.80 | 88.03 | 91.87 | 89.85 | 89.71 | 92.75 |
| | Sd | 0.00 | 0.40 | 1.57 | 1.14 | 1.58 | 2.82 | 1.56 | 1.54 | 1.40 |
| Dexamethasone | 1 | 100 | 94.97487 | 96.48241 | 88.44221 | 88.44221 | 89.44724 | 89.94975 | 89.44724 | 91.9598 |
| 1 mg/kg IP | 2 | 100 | 94.08867 | 95.5665 | 86.2069 | 86.69951 | 85.22167 | 85.22167 | 86.69951 | 88.17734 |
| (Group 6) | 3 | 100 | 95.07389 | 97.04433 | 91.62562 | 89.65517 | 88.66995 | 87.68473 | 88.17734 | 91.133* |
| | 4 | 100 | 96.31336 | 96.31336 | 86.17512 | 86.17512 | 87.5576 | 91.24424 | 91.24424 | 90.78341 |
| | 5 | 100 | 96.24413 | 96.71362 | 86.85446 | 86.85446 | 88.73239 | 88.73239 | 88.26291 | 90.14085 |
| | 6 | 100 | 96.41026 | 97.4359 | 89.74359 | 89.74359 | 90.25641 | 91.28205 | 89.23077 | 89.23077 |
| | 7 | 100 | 96.1165 | 96.60194 | 89.32039 | 86.40777 | 88.83495 | 87.86408 | 88.34951 | 89.32039 |
| | Av | 100.00 | 95.60** | 96.59 | 88.34 | 87.71 | 88.39 | 88.85 | 88.77 | 90.11 |
| | Sd | 0.00 | 0.34 | 0.22 | 0.77 | 0.58 | 0.61 | 0.82 | 0.53 | 0.49 |
| Dexamethasone | 1 | 100 | 100 | 97.90576 | 90.57592 | 86.91099 | 89.00524 | 90.05236 | 91.09948 | 93.19372 |
| 1 mg/kg IA | 2 | 100 | 97.53695 | 96.55172 | 91.133 | 87.68473 | 83.25123 | 83.25123 | 92.61084 | 99.01478 |
| (Group 7) | 3 | 100 | 95.69892 | 93.01075 | 88.70968 | 86.02151 | 86.02151 | 87.63441 | 87.63441 | 90.32258 |
| | 4 | 100 | 99.47917 | 93.22917 | 100 | 102.0833 | 104.1667 | 104.6875 | 104.1667 | 110.4167 |
| | 5 | 100 | 98.51485 | 95.0495 | 90.09901 | 87.62376 | 86.13861 | 89.10891 | 90.09901 | 94.55446 |
| | 6 | 100 | 97.52475 | 94.55446 | 87.62376 | 84.65347 | 78.21782 | 81.18812 | 90.09901 | 94.55446 |
| | 7 | 100 | 98.56459 | 96.17225 | 90.90909 | 85.64593 | 89.47368 | 90.43062 | 92.82297 | 96.17225 |
| | Av | 100.00 | 98.19 | 95.21 | 91.29 | 88.66 | 88.04 | 89.48 | 92.65 | 96.89 |
| | Sd | 0.00 | 0.54 | 0.68 | 1.53 | 2.27 | 3.05 | 2.86 | 2.03 | 2.47 |

*p < 0.05 vs. Vehicle (Group 1) using one-way ANOVA, followed by Dunnett's test.
**p < 0.01 vs. Vehicle (Group 1) using one-way ANOVA, followed by Dunnett's test.
***p < 0.001 vs. Vehicle (Group 1) using one-way ANOVA, followed by Dunnett's test.
****p < 0.0001 vs. Vehicle (Group 1) using one-way ANOVA, followed by Dunnett's test.

TABLE 35A
Paw thickness (mm) of treated leg per treatment group (Avg, S.D)

| Treatment | | | | | | | | | |
|---|---|---|---|---|---|---|---|---|---|
| Group/ | | | | | | | | | |
| Average paw | | | | | | | | | |
| thickness | | | | | | | | | |
| per day | Avg. | Avg. | Avg. | Avg | Avg. | Avg. | Avg. | Avg. | Avg. |
| (treated) | Baseline | Day 3 | Day 4 | Day 5 | Day 6 | Day 7 | Day 8 | Day 10 | Day 12 |
| BETi1 | 2.08 | 2.17 | 2.21 | 2.13 | 2.41 | 2.61 | 2.76 | 2.81 | 2.79 |
| Vehicle IA | | | | | | | | | |
| (Group 1) | | | | | | | | | |
| BETi1 | 2.12 | 2.16 | 2.24 | 2.16 | 2.17*** | 2.23** | 2.18** | 2.14** | 2.17 |
| 10 mg/kg IA | | | | | | | | | |
| (Group 2) | | | | | | | | | |
| BETi1 | 2.05 | 2.11 | 2.22 | 2.13 | 2.27* | 2.25** | 2.36* | 2.21* | 2.18 |
| 1 mg/kg IA | | | | | | | | | |
| (Group 3) | | | | | | | | | |
| BETi1 | 2.06 | 2.18 | 2.17 | 2.17 | 2.57* | 2.82 | 2.86 | 2.94 | 3.10 |
| 0.1 mg/kg IA | | | | | | | | | |
| (Group 4) | | | | | | | | | |
| BETi1 | 2.06 | 2.12 | 2.18 | 2.18 | 2.34 | 2.60 | 2.64 | 2.68 | 3.01 |
| 0.01 mg/kg IA | | | | | | | | | |
| (Group 5) | | | | | | | | | |
| Dexamethasone | 2.05 | 2.12 | 2.14 | 2.09 | 2.25** | 2.16**** | 2.12 | 2.09** | 2.07 |
| 1 mg/kg IP | | | | | | | | | |
| (Group 6) | | | | | | | | | |
| Dexamethasone | 1.98 | 2.13 | 2.16 | 2.15 | 2.24** | 2.30** | 2.44 | 2.45 | 2.50 |
| 1 mg/kg IA | | | | | | | | | |
| (Group 7) | | | | | | | | | |

| Treatment | | | | | | | | | |
|---|---|---|---|---|---|---|---|---|---|
| Group/ | | | | | | | | | |
| S.D. paw | | | | | | | | | |
| thickness | | | | | | | | | |
| per day | S.D. | S.D. | S.D. | S.D. | S.D. | S.D. | S.D. | S.D. | S.D. |
| (treated) | Baseline | Day 3 | Day 4 | Day 5 | Day 6 | Day 7 | Day 8 | Day 10 | Day 12 |
| BETi1 | 0.05 | 0.04 | 0.03 | 0.03 | 0.05 | 0.09 | 0.13 | 0.15 | 0.21 |
| Vehicle IA | | | | | | | | | |
| (Group 1) | | | | | | | | | |
| BETi1 | 0.04 | 0.04 | 0.03 | 0.02 | 0.02* | 0.05 | 0.04 | 0.01 | 0.03 |
| 10 mg/kg IA | | | | | | | | | |
| (Group 2) | | | | | | | | | |
| BETi1 | 0.03 | 0.01 | 0.03 | 0.02 | 0.02 | 0.04 | 0.12 | 0.07 | 0.05 |
| 1 mg/kg IA | | | | | | | | | |
| (Group 3) | | | | | | | | | |
| BETi1 | 0.03 | 0.04 | 0.03 | 0.02 | 0.04 | 0.07 | 0.08 | 0.11 | 0.13 |
| 0.1 mg/kg IA | | | | | | | | | |
| (Group 4) | | | | | | | | | |
| BETi1 | 0.03 | 0.02 | 0.02 | 0.02 | 0.04 | 0.08 | 0.11 | 0.21 | 0.26 |
| 0.01 mg/kg IA | | | | | | | | | |
| (Group 5) | | | | | | | | | |
| Dexamethasone | 0.04 | 0.02 | 0.03 | 0.03 | 0.02 | 0.02 | 0.04 | 0.04 | 0.04 |
| 1 mg/kg IP | | | | | | | | | |
| (Group 6) | | | | | | | | | |
| Dexamethasone | 0.02 | 0.02 | 0.02 | 0.02 | 0.04 | 0.06 | 0.13 | 0.17 | 0.29 |
| 1 mg/kg IA | | | | | | | | | |
| (Group 7) | | | | | | | | | |

*p < 0.05 vs. Vehicle (Group 1) using one-way ANOVA, followed by Dunnett's test.
**p < 0.01 vs. Vehicle (Group 1) using one-way ANOVA, followed by Dunnett's test.
***p < 0.001 vs. Vehicle (Group 1) using one-way ANOVA, followed by Dunnett's test.
****p < 0.0001 vs. Vehicle (Group 1) using one-way ANOVA, followed by Dunnett's test.

TABLE 35B
Paw thickness (mm) of untreated leg per treatment group (Avg., S.D)

| Treatment | | | | | | | | | |
|---|---|---|---|---|---|---|---|---|---|
| Group/Avg | | | | | | | | | |
| paw | | | | | | | | | |
| thickness | | | | | | | | | |
| per day | Avg. | Avg. | Avg. | Avg. | Avg. | Avg. | Avg. | Avg. | Avg. |
| (untreated) | Baseline | Day 3 | Day 4 | Day 5 | Day 6 | Day 7 | Day 8 | Day 10 | Day 12 |
| BETi1 | 2.14 | 2.09 | 2.15 | 2.13 | 2.40 | 2.72 | 2.87 | 2.83 | 3.00 |
| Vehicle IA | | | | | | | | | |
| (Group 1) | | | | | | | | | |
| BETi1 | 2.07 | 2.15 | 2.20 | 2.17 | 2.51 | 3.00 | 3.22 | 3.41 | 3.48 |
| 10 mg/kg IA | | | | | | | | | |
| (Group 2) | | | | | | | | | |
| BETi1 | 2.09 | 2.13 | 2.19 | 2.17 | 2.50 | 2.80 | 2.92 | 2.95 | 2.98 |
| 1 mg/kg IA | | | | | | | | | |
| (Group 3) | | | | | | | | | |
| BETi1 | 2.01 | 2.14 | 2.21 | 2.15 | 2.63 | 2.87 | 2.81 | 2.99 | 3.05 |
| 0.1 mg/kg IA | | | | | | | | | |
| (Group 4) | | | | | | | | | |
| BETi1 | 1.98 | 2.14 | 2.18 | 2.17 | 2.39 | 2.62 | 2.63 | 2.73 | 2.80 |
| 0.01 mg/kg IA | | | | | | | | | |
| (Group 5) | | | | | | | | | |
| Dexamethasone | 2.06 | 2.12 | 2.14 | 2.10 | 2.20 | 2.19 | 2.22 | 2.05 | 2.05 |
| 1 mg/kg IP | | | | | | | | | |
| (Group 6) | | | | | | | | | |
| Dexamethasone | 1.98 | 2.11 | 2.11 | 2.10 | 2.29 | 2.30 | 2.59 | 2.64 | 2.74 |
| 1 mg/kg IA | | | | | | | | | |
| (Group 7) | | | | | | | | | |
| Treatment | | | | | | | | | |
| Group/ | | | | | | | | | |
| S.D. paw | | | | | | | | | |
| thickness | | | | | | | | | |
| per day | S.D. | S.D. | S.D. | S.D. | S.D. | S.D. | S.D. | S.D. | S.D. |
| (untreated) | Baseline | Day 3 | Day 4 | Day 5 | Day 6 | Day 7 | Day 8 | Day 10 | Day 12 |
| BETi1 | 0.04 | 0.03 | 0.04 | 0.03 | 0.05 | 0.13 | 0.16 | 0.18 | 0.25 |
| Vehicle IA | | | | | | | | | |
| (Group 1) | | | | | | | | | |
| BETi1 | 0.03 | 0.04 | 0.01 | 0.02 | 0.04 | 0.05 | 0.07 | 0.12 | 0.24 |
| 10 mg/kg IA | | | | | | | | | |
| (Group 2) | | | | | | | | | |
| BETi1 | 0.05 | 0.02 | 0.02 | 0.01 | 0.07 | 0.10 | 0.17 | 0.21 | 0.23 |
| 1 mg/kg IA | | | | | | | | | |
| (Group 3) | | | | | | | | | |
| BETi1 | 0.04 | 0.03 | 0.02 | 0.02 | 0.08 | 0.16 | 0.12 | 0.15 | 0.21 |
| 0.1 mg/kg IA | | | | | | | | | |
| (Group 4) | | | | | | | | | |
| BETi1 | 0.03 | 0.02 | 0.02 | 0.02 | 0.06 | 0.11 | 0.14 | 0.26 | 0.24 |
| 0.01 mg/kg IA | | | | | | | | | |
| (Group 5) | | | | | | | | | |
| Dexamethasone | 0.04 | 0.02 | 0.03 | 0.02 | 0.01 | 0.05 | 0.03 | 0.03 | 0.03 |
| 1 mg/kg IP | | | | | | | | | |
| (Group 6) | | | | | | | | | |
| Dexamethasone | 0.03 | 0.02 | 0.02 | 0.01 | 0.06 | 0.07 | 0.17 | 0.21 | 0.25 |
| 1 mg/kg IA | | | | | | | | | |
| (Group 7) | | | | | | | | | |

TABLE 35C
Paw thickness (mm) of A clinical score untreated -treated (Avg., S.D)

| Treatment | | | | | | | | | |
|---|---|---|---|---|---|---|---|---|---|
| Group/Avg | | | | | | | | | |
| paw thickness | Avg. | Avg. | Avg. | Avg. | Avg. | Avg. | Avg. | Avg. | Avg. |
| per day | Delta | Delta | Delta | Delta | Delta | Delta | Delta | Delta | Delta |
| (Delta) | Baseline | Day 3 | Day 4 | Day 5 | Day 6 | Day 7 | Day 8 | Day 10 | Day 12 |
| BETi1 | 0.07 | −0.08 | −0.06 | 0.00 | −0.02 | 0.11 | 0.11 | 0.01 | 0.22 |
| Vehicle IA | | | | | | | | | |
| (Group 1) | | | | | | | | | |
| BETi1 | −0.05 | −0.01 | −0.04 | 0.01 | 0.34*** | 0.77*** | 1.04*** | 1.27 | 1.32 |
| 10 mg/kg IA | | | | | | | | | |
| (Group 2) | | | | | | | | | |
| BETi1 | 0.04 | 0.02 | −0.03 | 0.04 | 0.23* | 0.56* | 0.56 | 0.74 | 0.80 |
| 1 mg/kg IA | | | | | | | | | |
| (Group 3) | | | | | | | | | |
| BETi1 | −0.04 | −0.05 | 0.04* | −0.02 | 0.06 | 0.05 | −0.05 | 0.05 | −0.05 |
| 0.1 mg/kg IA | | | | | | | | | |
| (Group 4) | | | | | | | | | |
| BETi1 | −0.08 | 0.02 | 0.00 | 0.00 | 0.05 | 0.02 | −0.01 | 0.05 | −0.20 |
| 0.01 mg/kg IA | | | | | | | | | |
| (Group 5) | | | | | | | | | |
| Dexamethasone | 0.02 | 0.00 | −0.01 | 0.01 | −0.05 | 0.03 | 0.10 | −0.04 | −0.02 |
| 1 mg/kg IP | | | | | | | | | |
| (Group 6) | | | | | | | | | |
| Dexamethasone | 0.00 | −0.02 | −0.06 | −0.05 | 0.05 | −0.01 | 0.14 | 0.19 | 0.24 |
| 1 mg/kg IA | | | | | | | | | |
| (Group 7) | | | | | | | | | |

| Treatment | | | | | | | | | |
|---|---|---|---|---|---|---|---|---|---|
| Group/ | | | | | | | | | |
| S.D. paw | | | | | | | | | |
| thickness | | | | | | | | | |
| per day | S.D. | S.D. | S.D. | S.D. | S.D. | S.D. | S.D. | S.D. | S.D. |
| (Delta) | Baseline | Day 3 | Day 4 | Day 5 | Day 6 | Day 7 | Day 8 | Day 10 | Day 12 |
| BETi1 | 0.06 | 0.05 | 0.02 | 0.02 | 0.04 | 0.10 | 0.07 | 0.18 | 0.30 |
| Vehicle IA | | | | | | | | | |
| (Group 1) | | | | | | | | | |
| BETi1 | 0.04 | 0.06 | 0.02 | 0.02 | 0.05 | 0.07 | 0.09 | 0.13 | 0.26 |
| 10 mg/kg IA | | | | | | | | | |
| (Group 2) | | | | | | | | | |
| BETi1 | 0.04 | 0.03 | 0.02 | 0.01 | 0.06 | 0.09 | 0.11 | 0.17 | 0.19 |
| 1 mg/kg IA | | | | | | | | | |
| (Group 3) | | | | | | | | | |
| BETi1 | 0.06 | 0.06 | 0.02 | 0.02 | 0.07 | 0.12 | 0.09 | 0.08 | 0.26 |
| 0.1 mg/kg IA | | | | | | | | | |
| (Group 4) | | | | | | | | | |
| BETi1 | 0.05 | 0.03 | 0.02 | 0.02 | 0.06 | 0.12 | 0.16 | 0.24 | 0.21 |
| 0.01 mg/kg IA | | | | | | | | | |
| (Group 5) | | | | | | | | | |
| Dexamethasone | 0.05 | 0.02 | 0.03 | 0.01 | 0.02 | 0.04 | 0.04 | 0.03 | 0.03 |
| 1 mg/kg IP | | | | | | | | | |
| (Group 6) | | | | | | | | | |
| Dexamethasone | 0.03 | 0.02 | 0.01 | 0.02 | 0.05 | 0.11 | 0.27 | 0.34 | 0.47 |
| 1 mg/kg IA | | | | | | | | | |
| (Group 7) | | | | | | | | | |

*p < 0.05 vs. Vehicle (Group 1) using one-way ANOVA, followed by Dunnett's test.
***p < 0.001 vs. Vehicle (Group 1) using one-way ANOVA, followed by Dunnett's test.

TABLE 35D
Paw Thickness (mm)Treated Paw (T) vs. Untreated (U) Paw per group for each day

| | Paw Thickness (mm) per day T vs. U | | | | | | | |
|---|---|---|---|---|---|---|---|---|
| | D 3 | D 4 | D 5 | D 6 | D 7 | D 8 | D 10 | D 12 |
| treatment group | U vs T | U vs T | U vs T | U vs T | U vs T | U vs T | U vs T | U vs T |
| Vehicle IA | 0.192 | 0.031* | 0.860 | 0.748 | 0.337 | 0.190 | 0.943 | 0.502 |
| BETi1 10 mg/kg IA | 0.903 | 0.083 | 0.633 | 0.001* | 0.000* | 0.000* | 0.000* | 0.004* |
| BETi1 1 mg/kg IA | 0.440 | 0.195 | 0.013* | 0.011* | 0.001* | 0.002* | 0.004* | 0.006* |
| BETi1 0.1 mg/kg IA | 0.423 | 0.135 | 0.394 | 0.461 | 0.721 | 0.639 | 0.554 | 0.847 |
| BETi1 0.01 mg/kg IA | 0.656 | 1.000 | 0.867 | 0.461 | 0.886 | 0.959 | 0.844 | 0.375 |
| Dex 1 mg/kg IP | 0.828 | 0.844 | 0.532 | 0.116 | 0.511 | 0.036* | 0.284 | 0.583 |
| Dex 1 mg/kg IA | 0.289 | 0.031* | 0.054 | 0.240 | 0.959 | 0.613 | 0.602 | 0.633 |

(p < 0.05 is marked with*)

TABLE 35E
Paw Thickness (mm) D12 compared to D0 per treatment group for the
treated paw (p < 0.05 is marked with*)

| | Paw Thickness (mm) |
|---|---|
| Treatment group | D12 vs. D0 |
| Vehicle IA | 0.010* |
| BETi1 10 mg/kg IA | 0.553 |
| BETi1 1 mg/kg IA | 0.053 |
| BETi1 0.1 mg/kg IA | 0.000* |
| BETi1 0.01 mg/kg IA | 0.011* |
| Dex 1 mg/kg IP | 0.749 |
| Dex 1 mg/kg IA | 0.124 |

TABLE 36A
Clinical score (points) per day of treated leg per treatment group and day (Avg, S.D)

| Treatment group/ | | | | | | | | | |
|---|---|---|---|---|---|---|---|---|---|
| Average clinical score | Avg. | Avg. | Avg. | Avg. | Avg. | Avg. | Avg. | Avg. | Avg. |
| per day (treated) | Baseline | Day 3 | Day 4 | Day 5 | Day 6 | Day 7 | Day 8 | Day 10 | Day 12 |
| BETI1 | 0.00 | 0.00 | 0.00 | 0.00 | 0.83 | 1.67 | 2.00 | 2.00 | 2.17 |
| Vehicle IA (Group 1) | | | | | | | | | |
| BETI1 | 0.00 | 0.00 | 0.00 | 0.00 | 0.00* | 0.50* | 0.83 | 0.17* | 0.67 |
| 10 mg/kg IA (Group 2) | | | | | | | | | |
| BETI1 | 0.00 | 0.00 | 0.00 | 0.00 | 0.14 | 0.71 | 1.00 | 0.71 | 0.57 |
| 1 mg/kg IA (Group 3) | | | | | | | | | |
| BETI1 | 0.00 | 0.00 | 0.00 | 0.00 | 1.57** | 2.00 | 2.29 | 2.43 | 2.57 |
| 0.1 mg/kg IA (Group 4) | | | | | | | | | |
| BETI1 | 0.00 | 0.00 | 0.00 | 0.00 | 0.71 | 1.86 | 1.86 | 2.14 | 2.43 |
| 0.01 mg/kg IA (Group 5) | | | | | | | | | |
| Dexamethasone | 0.00 | 0.00 | 0.00 | 0.00 | 0.00* | 0.00** | 0.00** | 0.14* | 0.00* |
| 1 mg/kg IP (Group 6) | | | | | | | | | |
| Dexamethasone | 0.00 | 0.00 | 0.00 | 0.00 | 0.29 | 0.86 | 1.14 | 1.14 | 1.29 |
| 1 mg/kg IA (Group 7) | | | | | | | | | |
| Treatment Group/ | | | | | | | | | |
| S.D. paw thickness | S.D. | S.D. | S.D. | S.D. | S.D. | S.D. | S.D. | S.D. | S.D. |
| per day (treated) | Baseline | Day 3 | Day 4 | Day 5 | Day 6 | Day 7 | Day 8 | Day 10 | Day 12 |
| BETI1 | 0.00 | 0.00 | 0.00 | 0.00 | 0.31 | 0.21 | 0.52 | 0.52 | 0.60 |
| Vehicle IA (Group 1) | | | | | | | | | |
| BETI1 | 0.00 | 0.00 | 0.00 | 0.00 | 0.00 | 0.34 | 0.17 | 0.17 | 0.21 |
| 10 mg/kg IA (Group 2) | | | | | | | | | |
| BETI1 | 0.00 | 0.00 | 0.00 | 0.00 | 0.14 | 0.36 | 0.31 | 0.36 | 0.30 |
| 1 mg/kg IA (Group 3) | | | | | | | | | |
| BETI1 | 0.00 | 0.00 | 0.00 | 0.00 | 0.20 | 0.00 | 0.18 | 0.30 | 0.43 |
| 0.1 mg/kg IA (Group 4) | | | | | | | | | |
| BETI1 | 0.00 | 0.00 | 0.00 | 0.00 | 0.18 | 0.26 | 0.26 | 0.55 | 0.53 |
| 0.01 mg/kg IA (Group 5) | | | | | | | | | |
| Dexamethasone | 0.00 | 0.00 | 0.00 | 0.00 | 0.00 | 0.00 | 0.00 | 0.14 | 0.00 |
| 1 mg/kg IP (Group 6) | | | | | | | | | |
| Dexamethasone | 0.00 | 0.00 | 0.00 | 0.00 | 0.18 | 0.26 | 0.46 | 0.51 | 0.61 |
| 1 mg/kg IA (Group 7) | | | | | | | | | |

*p < 0.05 vs. Vehicle (Group 1) using one-way ANOVA, followed by Dunnett's test.
**p < 0.01 vs. Vehicle (Group 1) using one-way ANOVA, followed by Dunnett's test.

TABLE 36B
Clinical score (points) per day of untreated leg per treatment group and day (Avg., S.D)

| Treatment Group/ | | | | | | | | | |
|---|---|---|---|---|---|---|---|---|---|
| Average clinical score | Avg. | Avg. | Avg. | Avg. | Avg. | Avg. | Avg. | Avg. | Avg. |
| per day (untreated) | Baseline | Day 3 | Day 4 | Day 5 | Day 6 | Day 7 | Day 8 | Day 10 | Day 12 |
| BETI1 | 0.00 | 0.00 | 0.00 | 0.00 | 0.83 | 1.33 | 2.33 | 2.33 | 2.83 |
| Vehicle IA (Group 1) | | | | | | | | | |
| BETI1 | 0.00 | 0.00 | 0.00 | 0.00 | 0.83 | 1.83 | 3.00 | 3.00 | 3.33 |
| 10 mg/kg IA (Group 2) | | | | | | | | | |
| BETI1 | 0.00 | 0.00 | 0.00 | 0.00 | 0.71 | 1.43 | 2.43 | 2.00 | 2.43 |
| 1 mg/kg IA (Group 3) | | | | | | | | | |
| BETI1 | 0.00 | 0.00 | 0.00 | 0.00 | 1.14 | 2.00 | 2.29 | 2.57 | 3.00 |
| 0.1 mg/kg IA (Group 4) | | | | | | | | | |
| BETI1 | 0.00 | 0.00 | 0.00 | 0.00 | 0.86 | 1.57 | 1.71 | 2.14 | 1.43 |
| 0.01 mg/kg IA (Group 5) | | | | | | | | | |
| Dexamethasone | 0.00 | 0.00 | 0.00 | 0.00 | 0.00 | 0.00 | 0.00 | 0.00 | 0.00 |
| 1 mg/kg IP (Group 6) | | | | | | | | | |
| Dexamethasone | 0.00 | 0.00 | 0.00 | 0.00 | 0.57 | 0.86 | 1.71 | 1.57 | 1.71 |
| 1 mg/kg IA (Group 7) | | | | | | | | | |
| Treatment Group/ | | | | | | | | | |
| S.D. clinical per | S.D. | S.D. | S.D. | S.D. | S.D. | S.D. | S.D. | S.D. | S.D. |
| day (untreated) | Baseline | Day 3 | Day 4 | Day 5 | Day 6 | Day 7 | Day 8 | Day 10 | Day 12 |
| BETI1 | 0.00 | 0.00 | 0.00 | 0.00 | 0.31 | 0.33 | 0.49 | 0.49 | 0.60 |
| Vehicle IA (Group 1) | | | | | | | | | |
| BETI1 | 0.00 | 0.00 | 0.00 | 0.00 | 0.17 | 0.17 | 0.00 | 0.26 | 0.21 |
| 10 mg/kg IA (Group 2) | | | | | | | | | |
| BETI1 | 0.00 | 0.00 | 0.00 | 0.00 | 0.18 | 0.30 | 0.48 | 0.44 | 0.57 |
| 1 mg/kg IA (Group 3) | | | | | | | | | |
| BETi1 | 0.00 | 0.00 | 0.00 | 0.00 | 0.14 | 0.22 | 0.29 | 0.30 | 0.38 |
| 0.1 mg/kg IA (Group 4) | | | | | | | | | |
| BETI1 | 0.00 | 0.00 | 0.00 | 0.00 | 0.14 | 0.30 | 0.36 | 0.55 | 0.57 |
| 0.01 mg/kg IA (Group 5) | | | | | | | | | |
| Dexamethasone | 0.00 | 0.00 | 0.00 | 0.00 | 0.00 | 0.00 | 0.00 | 0.00 | 0.00 |
| 1 mg/kg IP (Group 6) | | | | | | | | | |
| Dexamethasone | 0.00 | 0.00 | 0.00 | 0.00 | 0.20 | 0.26 | 0.42 | 0.57 | 0.61 |
| 1 mg/kg IA (Group 7) | | | | | | | | | |

TABLE 36C
Δ clinical score (points) untreated -treated per treatment and day(Avg., S.D)

| Treatment | Avg. | Avg. | Avg. | Avg. | Avg. | Avg. | Avg. | Avg. | Avg. |
|---|---|---|---|---|---|---|---|---|---|
| Group/Average clinical | Delta | Delta | Delta | Delta | Delta | Delta | Delta | Delta | Delta |
| score per day (Delta) | Baseline | Day 3 | Day 4 | Day 5 | Day 6 | Day 7 | Day 8 | Day 10 | Day 12 |
| BETi1 | 0.00 | 0.00 | 0.00 | 0.00 | 0.00 | −0.33 | 0.33 | 0.33 | 0.67 |
| Vehicle IA (Group 1) | | | | | | | | | |
| BETi1 | 0.00 | 0.00 | 0.00 | 0.00 | 0.83* | 1.33* | 2.17* | 2.83* | 2.67 |
| 10 mg/kg IA (Group 2) | | | | | | | | | |
| BETi1 | 0.00 | 0.00 | 0.00 | 0.00 | 0.57 | 0.71 | 1.43 | 1.29 | 1.86 |
| 1 mg/kg IA (Group 3) | | | | | | | | | |
| BETi1 | 0.00 | 0.00 | 0.00 | 0.00 | −0.43 | 0.00 | 0.00 | 0.14 | 0.43 |
| 0.1 mg/kg IA (Group 4) | | | | | | | | | |
| BETi1 | 0.00 | 0.00 | 0.00 | 0.00 | 0.14 | −0.29 | −0.14 | 0.00 | −1.00 |
| 0.01 mg/kg IA (Group 5) | | | | | | | | | |
| Dexamethasone | 0.00 | 0.00 | 0.00 | 0.00 | 0.00 | 0.00 | 0.00 | −0.14 | 0.00 |
| 1 mg/kg IP (Group 6) | | | | | | | | | |
| Dexamethasone | 0.00 | 0.00 | 0.00 | 0.00 | 0.29 | 0.00 | 0.57 | 0.43 | 0.43 |
| 1 mg/kg IA (Group 7) | | | | | | | | | |
| Treatment Group/ | | | | | | | | | |
| S.D. clinical score | S.D. | S.D. | S.D. | S.D. | S.D. | S.D. | S.D. | S.D. | S.D. |
| per day (Delta) | Baseline | Day 3 | Day 4 | Day 5 | Day 6 | Day 7 | Day 8 | Day 10 | Day 12 |
| BETi1 | 0.00 | 0.00 | 0.00 | 0.00 | 0.00 | 0.33 | 0.21 | 0.21 | 0.49 |
| Vehicle IA (Group 1) | | | | | | | | | |
| BETi1 | 0.00 | 0.00 | 0.00 | 0.00 | 0.17 | 0.33 | 0.17 | 0.31 | 0.33 |
| 10 mg/kg IA (Group 2) | | | | | | | | | |
| BETi1 | 0.00 | 0.00 | 0.00 | 0.00 | 0.20 | 0.36 | 0.48 | 0.36 | 0.51 |
| 1 mg/kg IA (Group 3) | | | | | | | | | |
| BETi1 | 0.00 | 0.00 | 0.00 | 0.00 | 0.20 | 0.22 | 0.31 | 0.14 | 0.37 |
| 0.1 mg/kg IA (Group 4) | | | | | | | | | |
| BETi1 | 0.00 | 0.00 | 0.00 | 0.00 | 0.14 | 0.47 | 0.46 | 0.76 | 0.53 |
| 0.01 mg/kg IA (Group 5) | | | | | | | | | |
| Dexamethasone | 0.00 | 0.00 | 0.00 | 0.00 | 0.00 | 0.00 | 0.00 | 0.14 | 0.00 |
| 1 mg/kg IP (Group 6) | | | | | | | | | |
| Dexamethasone | 0.00 | 0.00 | 0.00 | 0.00 | 0.18 | 0.44 | 0.69 | 0.97 | 1.09 |
| 1 mg/kg IA (Group 7) | | | | | | | | | |

*p < 0.05 vs. Vehicle (Group 1) using one-way ANOVA, followed by Dunnett's test.

TABLE 36D
Clinical Score (points)Treated (T) Paw vs. Untreated
(U) Paw per treatment group for each day

| | Clinical score (points) per day T vs. U | | | | | | | |
|---|---|---|---|---|---|---|---|---|
| | D 3 | D 4 | D 5 | D 6 | D 7 | D 8 | D 10 | D 12 |
| Treatment Group | U vs T | U vs T | U vs T | U vs T | Uvs T | U vs T | U vs T | U vs T |
| Vehicle IA | 1.000 | 1.000 | 1.000 | 1.000 | 0.363 | 0.175 | 0.175 | 0.235 |
| BETi1 10 mg/kg IA | 1.000 | 1.000 | 1.000 | 0.004* | 0.010* | 0.000* | 0.000* | 0.000* |
| BETi1 1 mg/kg IA | 1.000 | 1.000 | 1.000 | 0.030* | 0.094 | 0.025* | 0.012* | 0.011* |
| BETi1 0.1 mg/kg IA | 1.000 | 1.000 | 1.000 | 0.078 | 1.000 | 1.000 | 0.356 | 0.289 |
| BETi1 0.01 mg/kg IA | 1.000 | 1.000 | 1.000 | 0.356 | 0.569 | 0.766 | 1.000 | 0.111 |
| Dex 1 mg/kg IP | 1.000 | 1.000 | 1.000 | 1.000 | 1.000 | 1.000 | 0.356 | 1.000 |
| Dex 1 mg/kg IA | 1.000 | 1.000 | 1.000 | 0.172 | 1.000 | 0.436 | 0.675 | 0.707 |

(p < 0.05 is marked with*)

TABLE 36E
Clinical score D12 compared to D0 per treatment group
for the treated paw (p < 0.05 is marked with*)

| | Avg. Clinical Score |
|---|---|
| Treatment Group | (points) D12 vs. D0 |
| Vehicle IA | 0.015* |
| BETi1 10 mg/kg IA | 0.025* |
| BETi1 1 mg/kg IA | 0.103 |
| BETi1 0.1 mg/kg IA | 0.001* |
| BETi1 0.01 mg/kg IA | 0.004* |
| Dex 1 mg/kg IP | 1.000 |
| Dex 1 mg/kg IA | 0.078 |

TABLE 36F
Histology

| | | Animal | Soft | Joint | | | |
|---|---|---|---|---|---|---|---|
| Group # | Paw | # | tissue | space | Cartilage | Bones | Score |
| Vehicle IA | R | 1 | 3.5 | 4 | 3 | 3 | 3.5 |
| (Group 1) | | 2 | 4 | 4 | 3.5 | 3.5 | 4 |
| | | 3 | 4 | 4 | 4 | 3.5 | 4 |
| | | 4 | 0 | 0 | 0 | 0 | 0 |
| | | 5 | 3.5 | 4 | 3.5 | 3.5 | 3.5 |
| | | 6 | 3.5 | 4 | 3.5 | 3.5 | 3.5 |
| | MEAN | | | | | | 3.08 |
| | S.D | | | | | | 0.62 |
| | L | 1 | 3.5 | 4 | 3.5 | 3.5 | 3.5 |
| | | 2 | 4 | 4 | 4 | 3.5 | 4 |
| | | 3 | 4 | 4 | 4 | 4 | 4 |
| | | 4 | 1.5 | 0 | 0 | 0 | 1 |
| | | 5 | 3.5 | 4 | 3.5 | 3.5 | 3.5 |
| | | 6 | 4 | 4 | 4 | 4 | 4 |
| | MEAN | | | | | | 3.33 |
| | S.D | | | | | | 0.48 |
| BETi1 | R | 1 | 4 | 4 | 4 | 4 | 4 |
| 10 mg/kg IA | | 2 | 4 | 4 | 4 | 4 | 4 |
| (Group 2) | | 3 | 3.5 | 4 | 4 | 4 | 4 |
| | | 5 | 3.5 | 4 | 4 | 4 | 4 |
| | | 6 | 4 | 4 | 4 | 4 | 4 |
| | | 7 | 4 | 4 | 4 | 4 | 4 |
| | MEAN | | | | | | 4.00 |
| | S.D | | | | | | 0.00 |
| | L | 1 | 0 | 0 | 0 | 0 | 0 |
| | | 2 | 0 | 0 | 0 | 0 | 0 |
| | | 3 | 0 | 0 | 0 | 0 | 0 |
| | | 5 | 0 | 0 | 0 | 0 | 0 |
| | | 6 | 1 | 0 | 0 | 0 | 1 |
| | | 7 | 1 | 0 | 0 | 0 | 1 |
| | MEAN | | | | | | 0.33**## |
| | S.D | | | | | | 0.21 |
| BETi1 | R | 1 | 4 | 4 | 4 | 4 | 4 |
| 1 mg/kg IA | | 2 | 1 | 0 | 0 | 0 | 1 |
| (Group 3) | | 3 | 4 | 4 | 4 | 4 | 4 |
| | | 4 | 3.5 | 4 | 4 | 4 | 4 |
| | | 5 | 4 | 4 | 4 | 3.5 | 4 |
| | | 6 | 3 | 4 | 4 | 4 | 4 |
| | | 7 | 3.5 | 4 | 4 | 4 | 4 |
| | MEAN | | | | | | 3.57 |
| | S.D | | | | | | 0.43 |
| | L | 1 | 3 | 4 | 3.5 | 3.5 | 3.5 |
| | | 2 | 2.5 | 4 | 2 | 2 | 2.5 |
| | | 3 | 1 | 0 | 0 | 0 | 1 |
| | | 4 | 1 | 0 | 0 | 0 | 1 |
| | | 5 | 3 | 4 | 3 | 3 | 3.5 |
| | | 6 | 0 | 0 | 0 | 0 | 0 |
| | | 7 | 2.5 | 4 | 4 | 4 | 3.5 |
| | MEAN | | | | | | 2.14 |
| | S.D | | | | | | 0.55 |
| Dexamethasone | R | 1 | 0 | 0 | 0 | 0 | 0 |
| 1 mg/kg IA | | 2 | 4 | 4 | 4 | 4 | 4 |
| (Group 7) | | 3 | 0 | 0 | 0 | 0 | 0 |
| | | 4 | 0 | 0 | 0 | 0 | 0 |
| | | 5 | 4 | 4 | 4 | 4 | 4 |
| | | 6 | 4 | 4 | 4 | 4 | 4 |
| | | 7 | 4 | 4 | 4 | 4 | 4 |
| | MEAN | | | | | | 2.29 |
| | S.D | | | | | | 0.81 |
| | L | 1 | 4 | 4 | 4 | 4 | 4 |
| | | 2 | 1.5 | 0 | 0 | 0 | 1 |
| | | 3 | 4 | 4 | 4 | 4 | 4 |
| | | 4 | 2 | 4 | 3 | 3 | 3 |
| | | 5 | 2 | 4 | 3 | 3 | 3 |
| | | 6 | 3 | 4 | 2.5 | 2.5 | 3 |
| | | 7 | 2.5 | 4 | 2.5 | 3 | 2.5 |
| | | MEAN | | | | | 2.93 |
| | | S.D | | | | | 0.38 |

Abbreviations: L—left, R—right, WNL—within normal limits
Scoring: 0 = within normal limits, 1—minimal, 2—mild, 3—moderate, 4—severe
The table indicates by + or − which tissues were affected in each sample.
**p < 0.01 using Student's T-test in comparison to the Vehicle Group (Group 1)
##p < 0.01 using Student's T-test in comparison to the contralateral hind paw (Right) of the same group

TABLE 37
Hot Plate Change in withdrawal latency (Sec)

| | Baseline | Baseline | Day 5 | Day 5 | Day 11 | Day 11 |
|---|---|---|---|---|---|---|
| AVG | (Avg.) | (S.D) | (Avg.) | (S.D) | (Avg.) | (S.D) |
| Vehicle IA | 12.14 | 1.03 | 12.83 | 1.14 | 9.83 | 1.56 |
| BETi1 10 mg/kg IA | 10.57 | 0.61 | 12.00 | 0.82 | 11.00 | 1.03 |
| BETi1 1 mg/kg IA | 11.71 | 0.42 | 11.57 | 0.72 | 11.29 | 1.27 |
| BETi1 0.1 mg/kg IA | 11.14 | 0.86 | 10.43 | 1.85 | 9.57 | 1.17 |
| BETi1 0.01 mg/kg IA | 11.57 | 1.00 | 11.86 | 1.70 | 12.00 | 1.43 |
| Dex 1 mg/kg IP | 11.86 | 0.51 | 12.43 | 1.09 | 12.29 | 1.95 |
| Dex 1 mg/kg IA | 11.71 | 1.08 | 10.14 | 1.55 | 12.86 | 1.20 |

### SUMMARY

In a rheumatoid arthritis model, BETi11, a pan-BET inhibitor, administered via intra-articular injection resulted in significant inhibition of inflammation in a validated animal model of RA. (Nandakumar K S, Holmdahl R. Collagen antibody induced arthritis. Methods Mol Med. 2007; 136:215-23.) BETi1 demonstrated statistically significant improvement in treatment response for both endpoints at 1 mg/kg and 10 mg/kg doses. BETi1 also demonstrated numerically superior results at 1 mg/kg and 10 mg/kg doses compared to leading injectable steroid, dexamethasone, in inhibiting inflammation in arthritic joints of mice. The data supports the potential to develop BETi1 as a locally-administered intra-articular joint treatment for an autoimmune joint disease.

In this validated preclinical model for rheumatoid arthritis, inflammatory arthritis was induced in BALB/c mice by systemically injecting a mixture of four arthritogenic monoclonal antibodies against collagen II at day 1. In addition, the mice received a lipopolysaccharide injection systemically at day 4 to stimulate an acute systemic inflammatory response. Each treatment group (n=7 per group) was injected with either (i) an intra-articular dose of BETi1 vehicle, (ii) an intra-articular dose of BETi1, (iii) an intra-articular dose of dexamethasone (1 mg/kg) or (iv) a systemic dose of dexamethasone (1 mg/kg, via intraperitoneal injection). The intra-articular doses were administered on days 0, 3, 6 and 9 while the dexamethasone systemic injections were given daily beginning at day 0 through 11. For the BETi1 treatment groups, four doses of BETi1 were evaluated (at concentrations ranging from 0.01 to 10 mg/kg). Each animal treated with the intra-articular injections received the injection in the ankle of one rear paw. The untreated rear paw was assessed to evaluate any potential anti-inflammatory systemic effect. Treatment response was evaluated based on an assessment of paw thickening or swelling (in millimeters) and arthritis scoring based on a five-point composite severity scale of redness, swelling of the ankles and wrists, and paw thickness. Scoring in this model ranges from 0 (normal) to 4 (extensive signs and symptoms of arthritis).

### Results

Paw Thickening: BETi1 demonstrated marked inhibition of paw thickening at the 1 and 10 mg/kg doses. At both doses, the inhibition of paw thickening was statistically significant in the treated paw relative to the untreated rear paw on day 12 (p<0.01).

For these BETi1 treatment groups, mean paw thickness at baseline (day 0) was 2.08 mm.

For the 10 mg/kg dose, the average paw thickness at day 12 was 3.48 mm in the untreated paw versus 2.17 mm in the treated paw, representing a 37.6% reduction.

For the 1 mg/kg dose, the average paw thickness at day 12 was 2.98 mm in the untreated paw versus 2.18 mm in the treated paw, representing a 26.8% reduction.

Results with these two doses were numerically superior to intra-articular dexamethasone and consistent with the systemic dexamethasone treatment.

Arthritis Score: Limbs treated with BETi1 at the 1 and 10 mg/kg dose levels had an average arthritis score of 0.57 and 0.67, respectively, or near normal. The arthritis score was significantly lower in the treated paw at both doses relative to the untreated paws on day 12 (p<0.05).

At the 10 mg/kg dose, the average arthritis score was 0.67 in the treated paw versus 3.33 in the untreated paw, representing a 79.9% reduction.

At the 1 mg/kg dose, the average arthritis score was 0.57 in the treated paw versus 2.43 in the untreated paw, representing a 76.5% reduction.

Limbs treated with intra-articular dexamethasone received an average arthritis score of 1.3, indicating mild symptoms. These animals had signs of redness and swelling of the ankle/wrist or apparent redness and swelling limited to individual digits.

Under the conditions of the study, a statistically significant reduction in the mean histopathological score was found following IA injection of BETi1 at a dose of 10 mg/kg (Group 2, left hind paw), in comparison to the contralateral hind paw or to the Vehicle group (Group 1), in an ACAIA mouse model.

### Locally Acting Anti-Inflammatory Effect:

Demonstrated improvement in signs and symptoms of joint inflammation were observed in the limbs of animals treated with BETi1, and no treatment effect was observed in untreated limbs, suggesting a locally acting anti-inflammatory effect of BETi1. In contrast, animals that received systemic dexamethasone experienced a treatment effect in all limbs.

Treatment tolerability was evaluated based on changes in body weight. Animals treated with systemic dexamethasone experienced continued weight loss throughout the study while all other treatment groups experienced improved body weights following recovery from the RA stimulant.

### Example 6. Homogeneity of BETi1 Dispersions

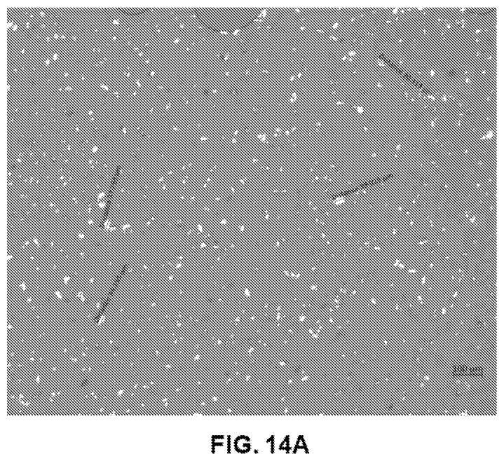

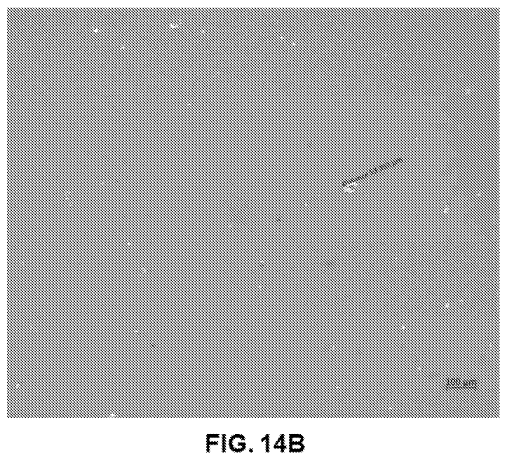

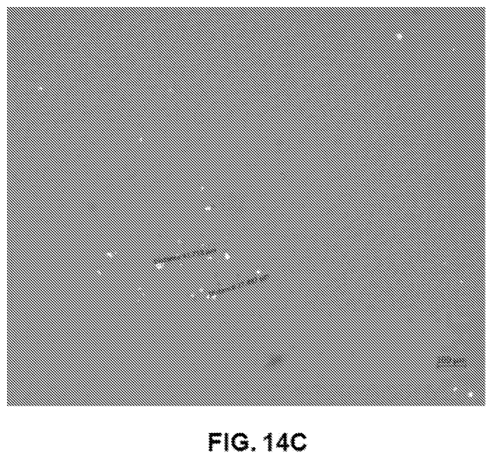

BETi suspensions at 4 mg/g (0.4%), at 0.4 mg/g (0.04%) and at 0.04 mg/g (0.004%) (Table 38) were prepared in NaCMC solutions as set out in Experimental Method N. The homogeneity (including absence of aggregates of solid particles) of the dispersion was analyzed and confirmed by light microscopy as shown in FIG. 14A-C. There were no visible aggregates of API particles for all four concentrations indicating that BETi1 was well dispersed. The number of crystals was significantly less for the 0.4 mg/g sample compared to the higher doses and some small crystals were observed. In the sample at 0.04 mg/g mainly larger crystals were observed, since the smaller ones were probably dissolved.

Based on solubility results shown in Example 8, it was estimated that in the sample of 0.04 mg/g (0.004%) approximately 90% of API is in undissolved form. For higher concentrations the undissolved material constitutes even higher fraction. For the sample at 0.004 mg/g (0.0004%) the API is dissolved or essentially or substantially dissolved.

TABLE 38

| | Formulation | Formulation | Formulation | Formulation | Vehicle |
|---|---|---|---|---|---|
| | #1 | #2 | #3 | #4 | #5 |
| | % w/w | | | | |
| BETI1 | 0.4 | 0.04 | 0.004 | 0.0004 | 0 |
| Na | 0.5 | 0.5 | 0.5 | 0.5 | 0.5 |
| CMC | | | | | |
| NaCl | 0.9 | 0.9 | 0.9 | 0.9 | 0.9 |
| Water | 98.2 | 98.56 | 98.596 | 98.5996 | 98.6 |

Solubility of BETi in water was determined to be about 0.005 mg/gram or 0.5 mg/100 ml H₂O (1 ml=1 g) using HPLC. The presence of low concentrations of saline and CMC in formulations #1-4 in Table 38 is not expected to have a significant effect on BETi1 solubility. Based on this the 0.0004 g/100 g formulation (i.e., 0.4 mg/100 ml) is dissolved or essentially or substantially dissolved in the saline and CMC formulation. About 12% of the 0.004 g/100 g (i.e., 4 mg/100 ml) formulation is dissolved. About 1.2% of the 0.04 g/100 g (i.e., 40 mg/100 ml) formulation is dissolved. About 0.12% of the 0.4 g/100 g (i.e., 400 mg/100 ml) formulation is dissolved. Without being bound by any theory BETi at 4 mg/100 ml, 40 mg/100 ml and 400 mg/100 ml may provide a reservoir effect as the solid dissolves over time.

### Example 7. BETi Doses Suitable for Intra-Articularly Administration in Humans

BETi dispersions for immediate and sustained release at different concentrations shown in Table 39A-B and Table 40A-B respectively are prepared. Tables 39A and 39B Prophetic Formulations for immediate release

TABLE 39A

| | For- | For- | For- | For- | For- |
|---|---|---|---|---|---|
| | mulation | mulation | mulation | mulation | mulation |
| | #8 | #9 | #10 | #11 | #12 |
| | % w/w | | | | |
| BETi1 | 1.0 | 1.0 | 1.0 | 0.1 | 10 |
| Na CMC | 0.5 | — | — | — | — |
| Methylcellulose | — | 0.5 | — | — | — |
| NaCl | 0.9 | 0.9 | 0.9 | 0.9 | 0.9 |
| Hyaluronic acid | 5.0 | — | — | — | — |
| Crosslinked | | 5.0 | 5.0 | 5.0 | 5.0 |
| Hyaluronic acid | | | | | |
| Benzyl alcohol | — | 1.0 | — | — | — |
| Water | 92.6 | 91.6 | 93.1 | 94.0 | 84.1 |

It should be noted that benzyl alcohol is an optional ingredient and can be replaced with other known in the art preservatives or avoided by sterilization.

TABLE 39B

| | Formulation | Formulation | Formulation | Formulation | Formulation | Formulation |
|---|---|---|---|---|---|---|
| | #13 | # 14 | # 15 | # 16 | # 17 | # 18 |
| | % w/w | % w/w | % w/w | % w/w | % w/w | % w/w |
| BETi1 | 0.5 | 0.5 | 0.5 | 0.5 | 0.5 | 0.5 |
| Na CMC | 0.5 | 0.5 | 0.5 | 0.5 | 0.5 | 0.5 |
| NaCl | 0.9 | 0.9 | 0.9 | 0.9 | 0.9 | 0.9 |
| Hyaluronic | 0.5 | 1.5 | 2.5 | 3.5 | — | — |
| acid | | | | | | |
| Carbopol | — | — | — | — | 1.5 | — |
| 934 | | | | | | |
| Xanthan | — | — | — | — | — | 1.5 |
| gum | | | | | | |
| Water | 97.6 | 96.6 | 95.6 | 94.6 | 96.6 | 96.6 |

TABLE 40A

| | For- | For- | For- | For- | For- |
|---|---|---|---|---|---|
| | mulation | mulation | mulation | mulation | mulation |
| | #19 | #20 | #21 | #22 | #23 |
| | % w/w | | | | |
| PLGA 75:25 | 5.0 | — | 2.5 | | 50 |
| microspheres | | | | | |
| containing 25% | | | | | |
| w/w BETi1 | | | | | |
| PLGA 50:50 | — | 5.0 | 2.5 | 0.5 | |
| microspheres | | | | | |
| containing 25% | | | | | |
| w/w BETi1 | | | | | |
| Na CMC | 0.5 | — | — | — | — |
| Methylcellulose | — | 0.5 | — | — | — |
| NaCl | 0.9 | 0.9 | 0.9 | 0.9 | 0.9 |
| Hyaluronic acid | 5.0 | — | — | — | — |
| Crosslinked | | 5.0 | 5.0 | 5.0 | 5.0 |
| Hyaluronic acid | | | | | |
| Benzyl alcohol | — | 1.0 | — | — | — |
| Water | 93.6 | 87.6 | 89.1 | 93.6 | 44.1 |

TABLE 40B

| | Formulation | Formulation | Formulation | Formulation | Formulation | Formulation |
|---|---|---|---|---|---|---|
| | #24 | # 25 | # 26 | # XX | # XX | # XX |
| | % w/w | % w/w | % w/w | % w/w | % w/w | % w/w |
| PLGA 75:25 | 5.0 | 5.0 | 5.0 | 5.0 | 5.0 | 5.0 |
| microspheres | | | | | | |
| containing | | | | | | |
| 25% w/w | | | | | | |
| BETi1 | | | | | | |
| Na CMC | 0.5 | 0.5 | 0.5 | 0.5 | 0.5 | 0.5 |
| NaCl | 0.9 | 0.9 | 0.9 | 0.9 | 0.9 | 0.9 |
| Hyaluronic | 0.5 | 1.5 | 2.5 | 3.5 | — | — |
| acid | | | | | | |
| Carbopol | — | — | — | — | 1.5 | — |
| 934 | | | | | | |
| Xanthan | — | — | — | — | — | 1.5 |
| gum | | | | | | |
| Water | 97.6 | 96.6 | 95.6 | 94.6 | 96.6 | 96.6 |

In one or more embodiments one or more of the following ingredients are suitable for use in a formulation to treat or prevent a joint disorder or disease and are compatible for use e.g., by injection into the joint e.g., intra articular, intra synovial, intra spinal and intra bursal, namely acetic acid, anhydrous trisodium citrate, benzalkonium chloride, benzyl alcohol, carboxymethylcellulose sodium, citric acid monohydrate, creatinine, dextrose, dibasic potassium phosphate, edetate disodium, hyaluronate sodium, hydrochloric acid, methylcellulose, methylparaben, miripirium chloride, monobasic potassium phosphate, phenol, phenylethyl alcohol, phosphoric acid, polyethylene glycol 3350, polyethylene glycol 4000, polyglactin, polysorbate 80, propylparaben, sodium acetate, sodium bisulfite, sodium chloride, sodium hydroxide, sodium metabisulfite, sodium phosphate, sodium phosphate, dibasic, anhydrous, sodium phosphate, dibasic, heptahydrate, sodium phosphate, monobasic, sodium phosphate, monobasic, monohydrate, sodium sulfite, sorbitol, and trisodium citrate dihydrate. In one or more embodiments one or more of ingredients or excipients suitable for use in a formulation to treat a joint disorder by injection as described herein may be added to the above formulations (Tables 39A and 39B and Tables 40A and 40B). In one or more embodiments one or more of these ingredients may be used to substitute for an ingredient in the above formulations, for example substituting a preservative for a different preservative or a salt for a different salt, or a polymeric agent for a different polymeric agent as would be appreciated by one skilled in the art. In some embodiments one or more of the above ingredients may be used in an immediate release formulation and in some embodiments one or more of the above ingredients may be used in a sustained release formulation as would be appreciated by one skilled in the art.

### Section D:

### Experimental Method P: Formulation Preparation

To make diluent: at ambient temperature, 100.00 g of the hydroxypropyl-β-cyclodextrin (2HIP-3-CD) in 900.0 mL of saline (0.9% w/v) solution is dissolved by mixing (magnetic stirrer or overhead mixer) for 5 minutes (mins) followed by sonication for 2 mins until 2HP-βCD—saline solution is clear and free of undissolved solids.

To make formulations #1-3: at ambient temperature, the indicated amount of BETi1 is transferred to a mixing vessel or container and 10% w/v of the diluent is added thereto and mixed (magnetic stirrer or overhead mixer) for 60 mins and then sonicated for 5 mins to disperse any aggregates. Thereafter the solutions are mixed for a further 30 minutes to ensure solution is clear and free of any undissolved solids resulting in formulations #1-3 respectively.

TABLE P

| | Formulation | Formulation | Formulation | Vehicle |
|---|---|---|---|---|
| | #1 | #2 | #3 | #4 |
| | % w/w | | | |
| BETI1 | 0.3 | 0.06 | 0.006 | 0 |
| Diluent | 10 | 10 | 10 | 10 |
| (2 hydroxypropyl-β- | | | | |
| cyclodextrin solution) | | | | |
| NaCl | 0.9 | 0.9 | 0.9 | 0.9 |
| Water | 88.8 | 89.04 | 89.094 | 89.1 |

### Experimental Method Q: Determination of Solubility of BETi1 in Solutions of Hydroxypropyl-β-Cyclodextrin in Saline

Saline is prepared by dissolving 0.9% of NaCl in water. 10% w/w or 20% w/w of hydroxypropyl-β-cyclodextrin (2HP-β-CD) is added to saline, sonicated for 1 minute, and mixed using magnetic stirrer until complete dissolution of 2HP-β-CD and a clear solution is obtained. Sufficient BETi1 is added to each 2HP-β-CD solution such that not all BETi1 is completely dissolved. The mixture is stirred with a magnetic stirrer at ambient conditions overnight (for at least 16 hours). The saturated samples, still containing solid phase, are filtered through a 0.45 μm filter and the filtrate is analyzed by HPLC.

Experimental Method R: Bleomycin Induced PF Mice Treated with BETi1 Formulations and Vehicle

Bleomycin, a non-heme iron protein, which acts inter alia by induction of DNA strand breaks. Bleomycin has been shown to induce lung injury and fibrosis in a wide variety of experimental animals including mice, rats, hamsters, rabbits, guinea pigs, dogs, and primates over a range of doses induced via intraperitoneal (ip), intravenous (iv), subcutaneous (sc), intratracheal (it), or intranasal (in) delivery. It has been suggested that bleomycin induces sensitivity to oxygen toxicity and recent studies support the role of the proinflammatory cytokines IL-18 and IL-1beta in the mechanism of bleomycin-induced lung injury.

The Bleomycin induced pulmonary fibrosis in a murine model is used to assess anti-fibrotic and/or anti-inflammatory properties of BETi1 formulations at three concentrations. Fifty C₅₇BL/6 male mice aged 6 to 7 weeks and weighing between 25-30 grams are randomized into five treatment groups (10 mice each): (a) bleomycin induced mice treated with BETi1 at three concentrations (0.06 mg/mL, 0.6 mg/mL and 3 mg/mL); (b) bleomycin induced mice treated with vehicle (control for active) and (c) uninduced mice treated with vehicle (sham) (control for disease).

To generate the morphological and functional aspects of PF, 50 uL bleomycin sulfate in saline per mouse is intranasally dosed on Day 0 (other than for group 1 sham receiving saline). The bleomycin IU conversion to enzyme units (U) is approximately 2 U/mg. For delivery of 4 U/kg in 50 μL, a solution of 2 U/ml (for 25 g mouse) is prepared in 0.9% NaCl.

5 ml of BETi1 solutions at 3 doses, or vehicle and bleomycin concomitantly are delivered once daily for 21 days with a nebulizer based aerosol delivery system (eFlow® rapid Nebuliser System) by inhalation for a period which allows the solutions to be nebulized and up to 2 hours per day. A sham group receives vehicle only. Body weights are recorded on days 1, 7, 14, and 21. Mortality and clinical observations are recorded daily.

On day 21, mice are euthanized by cervical dislocation and the whole lung is dissected and weighed. The chest is wet with alcohol and large scissors are used to open the ribcage. The lungs are removed with fine scissors and forceps, rinsed with PBS and patted dry on gauze.

The left lobe of lung tissue is collected, and snap frozen for hydroxyproline levels analysis. Hydroxyproline (4-hydroxyproline, Hyp) is a common nonproteinogenic amino acid. Hydroxyproline in tissue hydrolysates is an indirect measure of the amount of collagen present. Hydroxyproline content of lung tissue is measured by colorimetric assay.

The rest of lobes of lung tissue (including the superior lobe, middle-lobe, post-caval lobe and inferior lobe) are fixed for histopathology scoring. Seven mice from each group are randomly submitted for histopathology analysis (Hematoxylin and eosin (H & E) and Masson's Trichrome (MT)). The lungs are trimmed as needed and processed overnight in a Citadel 1000 automated processor by Shandon Scientific. Post-processing, lungs are embedded in paraffin blocks and sectioned in the following fashion: several slides are generated with lung sections in the first 1 mm of tissue, middle portions of the tissue, and finally near the latter third of the tissue in order to glean histological alterations throughout the lung. Sections are cut at 5 m thickness with a rotary microtome. Several slides per lung (at each depth) are stained with a H & E stain (reagents from Fisher Scientific) and MT Stain (Sigma kit HT15).

Pathological alterations are evaluated by a pathologist in blind manner (both dosage and treatment programs are unknown to the pathologist). The subacute interstitial inflammation and Agonal Hemorrhage are evaluated mainly based on the H & E staining. Fibrotic modifications are assessed morphologically and semi-quantitatively graded according to the scale defined by Ashcroft and modified by Hübner. Three sections for each lung sample are stained with MT and scored on a scale of 0-8. The final score is expressed as a mean of individual scores observed across all microscopic fields. In order to quantify the distribution of pulmonary fibrosis, the Ashcroft scores are graded in 3 general classes of increasing values: ranging from 0-3 (mild), 4 (moderate) and ≥5 (severe).

### Example 8. BETi Solubility

To characterize solubility of BETi1 in water, approximately 20 mg of BETi1 was placed into a glass vial with 10 milliliters of water and stirred with magnetic stirrer for 5 hours at ambient temperature. The resulting sample, still containing solid BETi1, was filtered and analyzed by HPLC for assay of BETi1 in solution. Solubility of BETi in water was determined to be about 0.005 mg/gram or 0.5 mg/100 mL water (1 ml=1 g) using HPLC (Table 41A).

The solubility of BETi1 was also determined in solutions of 2HP-β-CD in saline by saturation followed by HPLC analysis as set out in Method B. 10% HP-b-CD in saline can fully dissolve any amount of BETi1 below 4.3 mg/mL at room temperature (Table 41A).

To characterize the solubility of BETi in other solvents, about 5 mg of BETi1 was weighed into a 2 ml glass vial. 20 μL aliquots of each of the solvent listed in Table 41B was added to dissolve the drug substance at 25° C. Vortex and sonication were applied to assist dissolution. Approximate solubility was determined by visual observation and reported in

TABLE 41A
Approximate solubility of BETi1 at 25° C.

| Solvents | Solubility (mg/mL) |
|---|---|
| Water | 0.005 |
| 2HP-β-CD - Saline (20%:80%) | 8.99 |
| 2HP-β-CD - Saline (10%:90%) | 4.34 |

TABLE 41B
Approximate solubility of BETi1 at 25° C.

| Solvents | Solubility (mg/mL) |
|---|---|
| Methanol | 21 |
| Ethanol | 12 |
| Isopropanol | 6 |
| Acetone | 15 |
| Methyl ethyl ketone | 12 |
| Ethyl acetate | <5 |
| Acetonitrile | <5 |
| IPAc | <5 |
| Tetrahydrofuran | 104 |
| t-Butyl methyl ether | <5 |
| 1,4-Dioxane | 104 |
| Heptane | <5 |
| Dichloromethane | <5 |
| Anisole | <5 |

The solubility of BETi1 was also determined in polyethylene glycol 400, which is an oral and dermal compatible solvent, and found to be approximately 20 mg/ml. Solvents may be chosen to achieve a suspension or solutions and/or different levels of solubility. For example, solvents may be chosen to achieve a composition where all, or essentially, or substantially all the compound is in suspension. For example, solvents may be chosen to achieve about 0.1% solubility, where only about 0.1% of the compound is dissolved and the remainder is in suspension. For example, solvents may be chosen to achieve about 1% solubility, where only about 1% of the compound is dissolved and the remainder is in suspension. For example, solvents may be chosen to achieve about 10% solubility, where about 10% of the compound is dissolved and the remainder is in suspension. For example, solvents may be chosen to achieve about 99% solubility, where only about 99% of the compound is dissolved and the remainder is in suspension. For example, solvents may be chosen to achieve dissolution where all or essentially, or substantially all of the compound is dissolved. Solvents with a solubility of about 20 mg/mL or more, for example, can be chosen to solubilize higher concentrations of BETi compounds, such as 2%.

The presence of some soluble BETi compound in the formulation may in some embodiments help delivery and therapeutic effect. In some embodiments, without being bound by any theory as some of the solubilized BETi binds to BD domains or is metabolized a gradient is provided by which suspended BETi is driven into solution.

### Example 9. BETi1 Solutions

BETi solutions at 3 mg/mL (0.3%), at 0.6 mg/mL (0.06%) and at 0.06 mg/mL (0.006%) (Table 42) were prepared in 10% 2HP-β-CD in 0.9% saline as set out in Experimental Method P.

TABLE 42

| | Formulation | Formulation | Formulation | Vehicle |
|---|---|---|---|---|
| | #1 | #2 | #3 | #4 |
| | % w/w | | | |
| BETi1 | 0.3 | 0.06 | 0.006 | 0 |
| Diluent | 10 | 10 | 10 | 10 |
| (2 hydroxypropyl- | | | | |
| β-cyclodextrin) | | | | |
| NaCl | 0.9 | 0.9 | 0.9 | 0.9 |
| Water | 88.8 | 89.04 | 89.094 | 89.1 |

### Example 10. Bleomycin Induced IPF in Mice and Treatment with BETi1 Formulations

Investigation of the anti-fibrotic properties of BETi1 composition against TGF-β mediated fibrosis was undertaken in a bleomycin induced murine model mimicking IPF in accordance with the protocol set out in Experimental Method R.

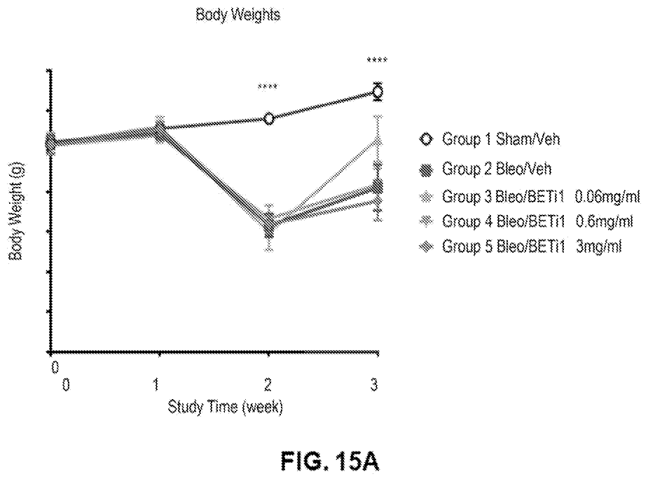

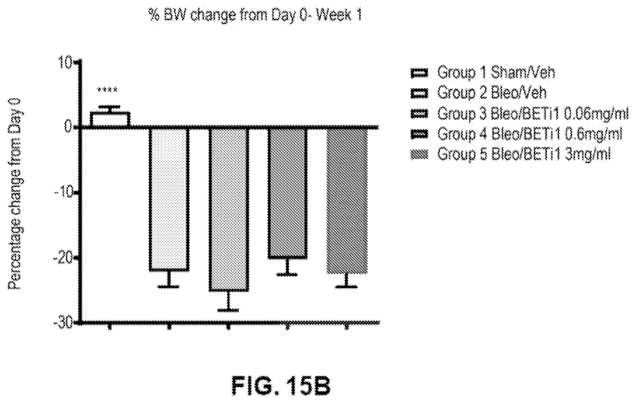

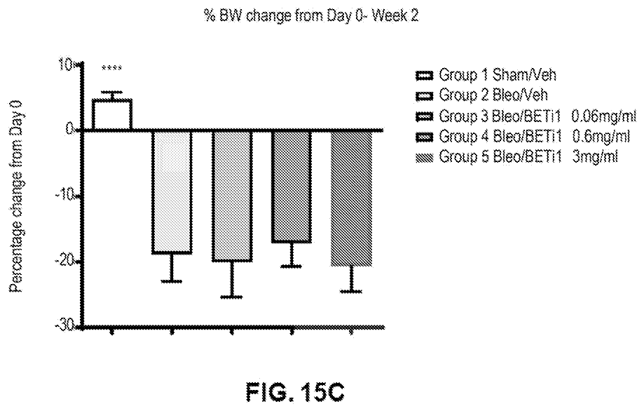

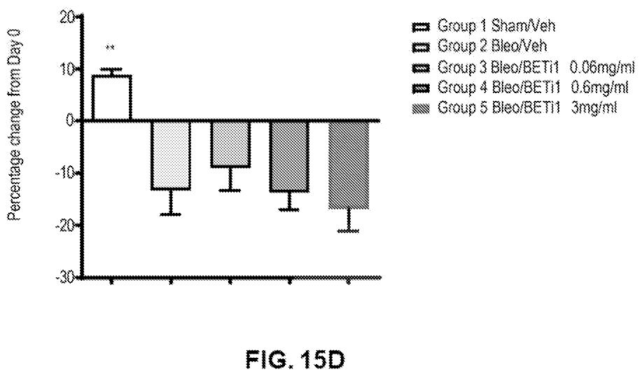

Uninduced animals receiving vehicle only (group 1 sham/veh) continue to gain weight normally throughout the study. The mean body weight for group 1 was statistically higher for week 2 and 3 compared to vehicle (group 2). Animals receiving Bleomycin and vehicle (group 2) or BETi (groups 3-5) lost weight from week 1 onwards. After one week and up to the end of the second week, animals receiving bleomycin and vehicle or BETi1 had a drop in body weight but started regaining weight from week 2. Whereas uninduced animals receiving vehicle (group 1 sham/veh) continued to gain weight normally throughout the study (Tables 43A-G and FIGS. 15A-D).

An inverse dose response for BETi1 treated animals (groups 3-5) (lower dose gains most weight verses higher doses) was observed (FIG. 15A). Without being bound to any theory, the inverse dose response for BETi1 treated animals may indicate that the lower doses are better tolerated. The percent change in body weight for sham/vehicle (group 1) was statistically significant higher compared to vehicle (group 2) throughout the study (FIG. 15B-D)

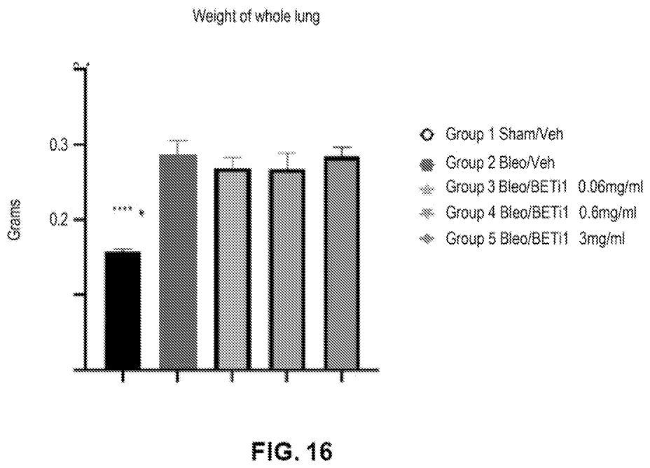

Animals receiving bleomycin and vehicle demonstrated a statistically significant increase in whole lung weight in comparison to uninduced animals receiving vehicle (approximately twice the weight of sham/vehicle). Treatment with BETi1 at 0.06 mg/mL and 0.6 mg/mL doses demonstrated a slight decrease in lung weight in comparison to animals receiving bleomycin and vehicle (Tables 44A-B. and FIG. 16).

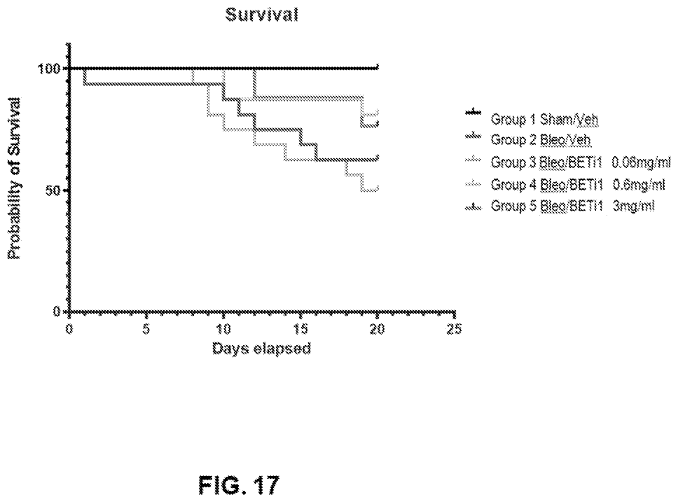

Animals receiving bleomycin and vehicle (62.5% probability of survival) or bleomycin and 0.06 mg/mL BETi1 (50% probability of survival) demonstrated higher mortality relative to the higher doses of BETi1 (0.6 mg/mL, 81.3% and 3 mg/mL 76.5% probability of survival respectively) (Table 45. and FIG. 17). Probability of survival is determined as the percent of animals in each group that survived until day 20. It should be noted that the survival endpoint is affected by significant variability and bias has an inherent limitation that even if treatment is being effective the bleomycin induced damage may have already been fatal to a substantial proportion of the animals as the study progresses. For example, in the bleomycin induced vehicle control animals a fatality occurred after a day. After about 10 days and thereafter the two highest BETi groups showed better survival rates whilst the lower concentration had a survival probability not dissimilar to the bleomycin induced vehicle treated control. On the other hand, subjects with PF would not have to contend with the effects of bleomycin and may therefore cope better with the disorder in the absence of bleomycin.

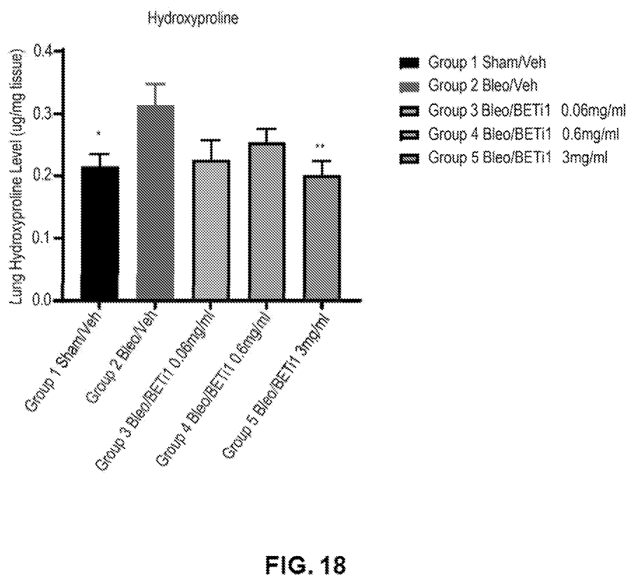

Animals receiving bleomycin and vehicle (group 2) demonstrated a statistically significant increase in hydroxyproline levels in comparison to uninduced animals receiving vehicle (group 1). Whereas treatment with BETi1 at 0.06 mg/mL (group 3), 0.6 mg/mL (group 4) and 3 mg/mL (group 5) demonstrated a decrease in hydroxyproline levels in comparison to animals receiving bleomycin and vehicle (group 2) (Tables 46A-B. and FIG. 18). Reduction in hydroxyproline levels in animals treated with BETi1 at 3 mg/mL (group 5) was statistically significant compared to treatment with vehicle (group 2).

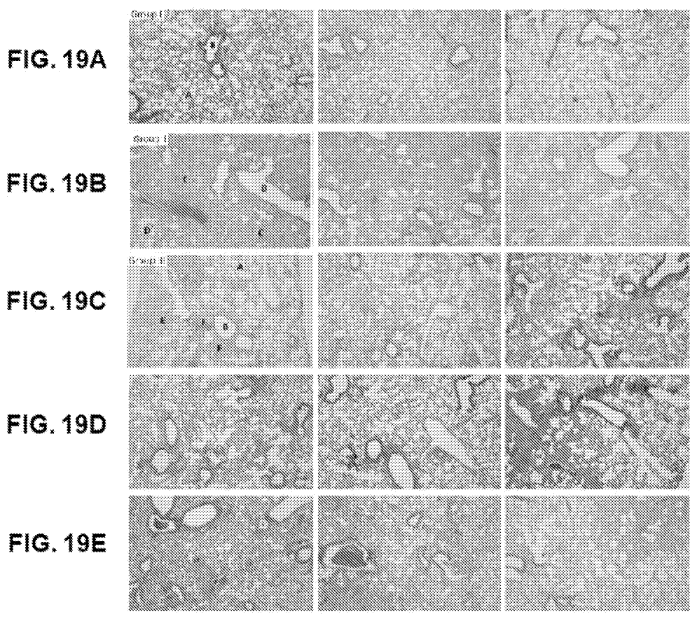

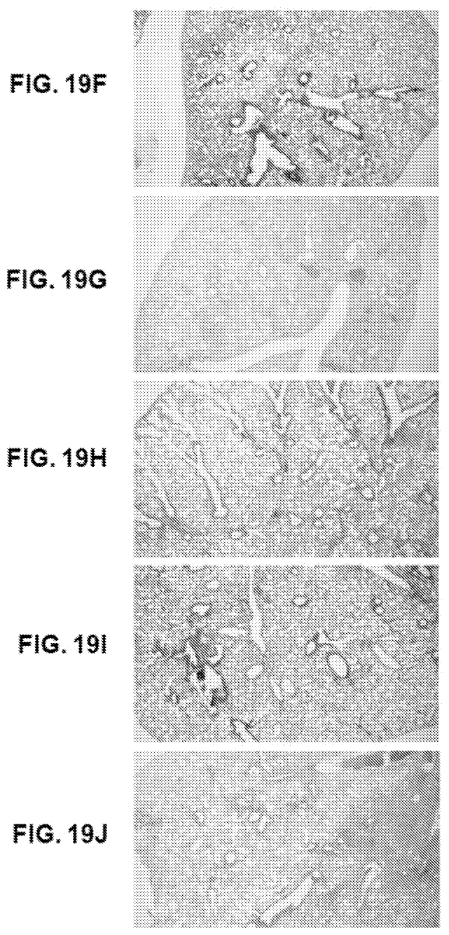

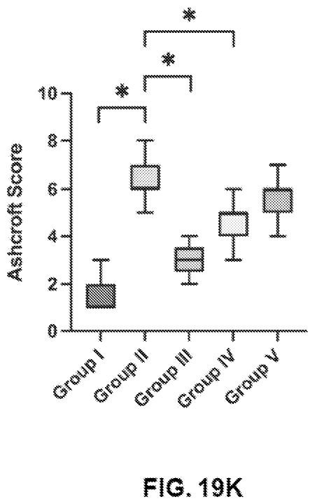

Three sections for each lung sample were stained with H & E and Masson's Trichrome and scored on a Hübner-modified Ashcroft scale of 0-8. Three pictorial representations for each of groups 1-5 are shown in FIGS. 19A-E respectively (100×) and one representation for each of groups 1-5 is shown in FIGS. 19F-J (40×). Histology provides a picture or snapshot of the absence or presence of fibrosis. As shown in FIG. 19A (left image) healthy animals sham control (group 1) demonstrated normal lung parenchyma with open airways and airspaces (area A) and small conducting airways (area B). In contrast animals receiving bleomycin and vehicle (group 2) demonstrated fibrotic lesions with abundant cellularity (area C), sporadic airways (area D) and large conducting airways (area B) (FIG. 19B (left image)). Treatment with BETi1 at 0.06 mg/mL (group 3) demonstrated significant areas of normal lung parenchyma with open airways and airspaces (area A) and normal conducting airways (area B). In addition, less severe localized fibrosis in parenchyma (area E) and less severe localized fibrosis in airway was observed in group 3 (FIG. 19C (left image)). Other groups with higher concentrations of BETi also appeared to lessen bleomycin induced fibrosis but to a lesser degree.

As shown in FIG. 19G (bleomycin and vehicle-group 2), fibrosis was not observed throughout the lung tissue. Without being bound by any theory, this may be due to a limitation of the study, wherein fixation was conducted on non-inflated lung tissue. Generally, lung tests that involve morphology are best shown following inflation-fixation. During such a technique, fixative is delivered through the tracheal canula approximately 25 cm above the mouse so that gravimetrically, inflation of fixative results in an inter-breath appearance (lung is not collapsed or artificially too expanded).

Histology scoring according to the Hübner-modified Ashcroft is shown in FIG. 19K. The final score was expressed as a mean of individual scores observed across all microscopic fields. Mean scores per group were as follows Group 1 (uninduced animals receiving vehicle): 1.52, Group 2 (animals receiving bleomycin and vehicle): 6.36, Group 3 (animals receiving bleomycin and BETi1 at 0.06 mg/mL): 3.00, Group 4 (animals receiving bleomycin and BETi1 at 0.6 mg/mL): 4.64, and Group 5 (animals receiving bleomycin and BETi1 at 3 mg/mL): 5.52. Significant differences were observed when the following groups were compared: Groups 1 vs. 2, Groups 2 vs. 3, and Groups 2 vs. 4 (*P≤0.05). Group 2 vs. Group 5 trended toward significance but was not significantly different at P≤0.05). Group 3 (0.06 mg/mL) had the greatest inhibitory effect on fibrotic tissue formation in the lung The groups that received 0.06 mg/mL and 0.6 mg/mL showed a reduction in fibrosis in the subjects of over about 50% and nearly about 30% respectively within the 21 day study. This may potentially translate over time into a significant delaying or retardation of the disease.

Compared to sham/vehicle control (Group 1), bleomycin induced a significant degree of lung fibrosis (group 2) observed in both H & E and Masson's Trichrome stained sections. Group 3 appears to inhibit bleomycin-induced fibrosis better than Group 4 or Group 5. It appears that significant lessening of fibrosis was observed with Groups receiving lower doses of BETi1, but further testing is warranted to support an inversed dose effect of BETi1 (lower doses having a greater inhibitory effect on fibrotic tissue than higher dose).

Histology is regarded as the primary data indicating a reduction or amelioration of fibrosis followed by or coupled with the hydroxyproline data and other data like body weight is informational. Overall, and taken together with hydroxyproline results, histopathological analysis by H & E (Fibrosis score) and Masson's trichrome staining (Ashcroft score) revealed that increased fibrosis as evidenced by collapse of alveolar spaces (airways and airspaces) due to excess deposition of collagen fibers (evidenced by Hydroxyproline) and fibrotic lesions were observed in Group 2 (animals receiving bleomycin and vehicle), whereas treatment with BETi1 ameliorated the bleomycin induced abnormal changes in the lungs. Treatment with inhaled BETi1 formulations attenuated the degree of bleomycin-induced fibrosis in lung tissues compared to vehicle. The histology observations and scores indicate that the BETi compounds can offer a useful method of treating, retarding or ameliorating PF in humans and a significantly increased life span for affected subjects.

TABLE 43A
% change in Body Weight Week 1 Compared to Baseline

| | Group 1 | Group 2 | Group 3 | Group 4 | Group 5 |
|---|---|---|---|---|---|
| Animal | Sham/ | Bleo/ | Bleo/BETi1 | Bleo/BETi1 | Bleo/BETi1 |
| # | Veh | Veh | 0.06 mg/mL | 0.6 mg/mL | 3 mg/mL |
| 1 | 4.76 | −27.91 | −18.53 | −10.36 | −10.16 |
| 2 | 3.83 | −28.99 | −13.95 | −31.41 | −15.64 |
| 3 | 4.10 | −27.68 | −12.29 | −8.41 | −21.86 |
| 4 | 4.61 | −12.96 | −37.04 | −10.00 | −26.87 |
| 5 | 2.48 | −24.87 | −20.00 | −30.77 | −23.22 |
| 6 | −3.18 | −19.74 | −39.32 | −22.17 | −36.71 |
| 7 | 3.59 | −17.31 | −11.86 | −31.64 | −15.25 |
| 8 | 1.87 | −18.65 | −37.37 | −20.93 | −13.81 |
| 9 | 0.00 | −33.65 | −3.83 | −27.01 | −12.78 |
| 10 | 1.91 | −20.31 | −26.78 | −24.89 | −17.30 |
| 11 | | −7.01 | −31.75 | −31.19 | −34.80 |
| 12 | | −10.10 | −37.30 | −5.26 | −28.32 |
| 13 | | −22.87 | −38.92 | −14.75 | −27.55 |
| 14 | | −37.37 | −35.61 | −23.22 | −24.87 |
| 15 | | −35.27 | −25.13 | −25.78 | −34.38 |
| 16 | | −9.01 | −14.61 | −4.72 | −26.99 |
| 17 | | | | | −11.63 |
| mean | 2.40 | −23.21 | −22.10 | −21.76 | −19.36 |
| s.d. | 2.33 | 9.12 | 11.42 | 9.47 | 8.29 |

TABLE 43B
% change in Body Weight week 2 Compared to Baseline

| | Group 1 | Group 2 | Group 3 | Group 4 | Group 5 |
|---|---|---|---|---|---|
| Animal | Sham/ | Bleo/ | Bleo/BETi1 | Bleo/BETi1 | Bleo/BETi1 |
| # | Veh | Veh | 0.06 mg/mL | 0.6 mg/mL | 3 mg/mL |
| 1 | 3.81 | −41.86 | −19.83 | −0.90 | −1.07 |
| 2 | 5.74 | −39.61 | −3.26 | −39.27 | −7.11 |
| 3 | 7.69 | | −7.63 | −2.34 | −20.47 |
| 4 | 9.68 | −5.09 | −39.35 | −8.00 | −11.45 |
| 5 | 6.44 | −10.66 | −15.35 | −33.52 | −36.97 |
| 6 | 0.91 | −16.74 | −47.44 | −16.04 | −40.10 |
| 7 | 8.52 | −7.69 | −1.27 | | −20.18 |
| 8 | −1.40 | −22.28 | | −27.44 | 1.43 |
| 9 | 3.29 | −35.10 | −1.28 | −32.70 | −7.05 |
| 10 | 2.87 | −5.73 | −37.16 | −21.78 | −22.16 |
| 11 | | −4.21 | | | −38.24 |
| 12 | | | | 0.96 | −34.07 |
| 13 | | −37.23 | | −18.89 | |
| 14 | | | | −16.11 | −20.81 |
| 15 | | −11.16 | −39.04 | −23.11 | |
| 16 | | −7.66 | −8.22 | −0.47 | −42.92 |
| 17 | | | | | −8.37 |
| mean | 4.75 | −18.85 | −19.98 | −17.12 | −20.64 |
| s.d. | 3.32 | 13.95 | 16.73 | 12.93 | 14.39 |

TABLE 43C
% change in Body Weight week 3 Compared to Baseline

| | Group 1 | Group 2 | Group 3 | Group 4 | Group 5 |
|---|---|---|---|---|---|
| Animal | Sham/ | Bleo/ | Bleo/BETi1 | Bleo/BETi1 | Bleo/BETi1 |
| # | Veh | Veh | 0.06 mg/mL | 0.6 mg/mL | 3 mg/mL |
| 1 | 8.10 | −2.78 | −14.22 | −9.91 | 2.67 |
| 2 | 9.57 | −0.51 | 0.93 | | −4.27 |
| 3 | 9.74 | −12.45 | −2.54 | −0.47 | −18.14 |
| 4 | 15.21 | −6.25 | −37.04 | −7.50 | −5.73 |
| 5 | 7.92 | −32.12 | −9.77 | −34.62 | −37.44 |
| 6 | 5.45 | −39.42 | | −7.55 | |
| 7 | 14.35 | 2.60 | −0.85 | | −21.52 |
| 8 | 2.34 | −0.93 | | −27.91 | 1.90 |
| 9 | 7.51 | | −2.13 | −34.12 | −7.05 |
| 10 | 7.66 | −38.83 | | −12.89 | −24.32 |
| 11 | | | | | −46.08 |
| 12 | | −9.38 | | 2.39 | −37.17 |
| 13 | | −4.50 | −5.02 | −6.45 | |
| 14 | | | | −14.69 | −17.26 |
| 15 | | | | −19.56 | |
| 16 | | | | −3.30 | |
| 17 | | | | | −3.26 |
| Mean | 8.78 | −13.14 | −8.83 | −13.58 | −16.74 |
| S.D | 3.61 | 15.11 | 11.64 | 11.69 | 15.36 |

TABLE 43D
% Change Body Weight One Way ANOVA Week 1 multiple comparisons

| Dunnett's multiple | Mean | 95.00% | Below | | Adjusted | | |
|---|---|---|---|---|---|---|---|
| comparisons test | Diff. | CI of diff. | threshold? | Summary | P Value | B¹ | |
| Group 2 Bleo/Veh vs. | −24.50 | −33.91 | Yes | **** | <0.0001 | A | Group 1 |
| Group 1 Sham/Veh | | to −15.10 | | | | | Sham/Veh |
| Group 2 Bleo/Veh vs. | 3.162 | −5.088 | No | ns | 0.7462 | C | Group 3 |
| Group 3 Bleo/BETI1 | | to 11.41 | | | | | Bleo/BETi1 |
| 0.06 mg/mL | | | | | | | 0.06 mg/mL |
| Group 2 Bleo/Veh vs. | −1.950 | −10.20 | No | ns | 0.9395 | D | Group 4 |
| Group 4 Bleo/BETI1 | | to 6.300 | | | | | Bleo/BETi1 |
| 0.6 mg/mL | | | | | | | 0.6 mg/mL |
| Group 2 Bleo/Veh vs. | 0.3723 | −7.755 | No | ns | 0.9998 | E | Group 5 |
| Group 5 Bleo/BETI1 | | to 8.500 | | | | | Bleo/BETi1 |
| 3 mg/mL | | | | | | | 3 mg/mL |

| | | | Mean | SE of | | | | |
|---|---|---|---|---|---|---|---|---|
| Test details | Mean 1 | Mean 2 | Diff. | diff. | n1 | n2 | q | DF |
| Group 2 Bleo/Veh vs. | −22.11 | 2.396 | −24.50 | 3.755 | 16 | 10 | 6.525 | 70 |
| Group 1 Sham/Veh | | | | | | | | |
| Group 2 Bleo/Veh vs. | −22.11 | −25.27 | 3.162 | 3.293 | 16 | 16 | 0.9601 | 70 |
| Group 3 Bleo/BETi1 | | | | | | | | |
| 0.06 mg/mL | | | | | | | | |
| Group 2 Bleo/Veh vs. | −22.11 | −20.16 | −1.950 | 3.293 | 16 | 16 | 0.5920 | 70 |
| Group 4 Bleo/BETi1 | | | | | | | | |
| 0.6 mg/mL | | | | | | | | |
| Group 2 Bleo/Veh vs. | −22.11 | −22.48 | 0.3723 | 3.245 | 16 | 17 | 0.1147 | 70 |
| Group 5 Bleo/BETi1 | | | | | | | | |
| 3 mg/mL | | | | | | | | |

¹Each group is compared to group B (group 2 vehicle).

TABLE 43E
% Change Body Weight One Way ANOVA Week 2 multiple comparisons

| Dunnett's multiple | Mean | 95.00% | Below | | Adjusted | | |
|---|---|---|---|---|---|---|---|
| comparisons test | Diff. | CI of diff. | threshold? | Summary | P Value | B² | |
| Group 2 Bleo/Veh vs. | −23.60 | −38.28 | Yes | *** | 0.0006 | A | Group 1 |
| Group 1 Sham/Veh | | to −8.928 | | | | | Sham/Veh |
| Group 2 Bleo/Veh vs. | 1.135 | −13.16 | No | ns | 0.9988 | C | Group 3 |
| Group 3 Bleo/BETi1 | | to 15.43 | | | | | Bleo/BETi1 |
| 0.06 mg/mL | | | | | | | 0.06 mg/mL |
| Group 2 Bleo/Veh vs. | −1.732 | −15.17 | No | ns | 0.9929 | D | Group 4 |
| Group 4 Bleo/BETi1 | | to 11.70 | | | | | Bleo/BETi1 |
| 0.6 mg/mL | | | | | | | 0.6 mg/mL |
| Group 2 Bleo/Veh vs. | 1.788 | −11.43 | No | ns | 0.9914 | E | Group 5 |
| Group 5 Bleo/BETi1 | | to 15.01 | | | | | Bleo/BETi1 |
| 3 mg/mL | | | | | | | 3 mg/mL |

| | | | Mean | SE of | | | | |
|---|---|---|---|---|---|---|---|---|
| Test details | Mean 1 | Mean 2 | Diff. | diff. | n1 | n2 | q | DF |
| Group 2 Bleo/Veh vs. | −18.85 | 4.754 | −23.60 | 5.836 | 13 | 10 | 4.044 | 58 |
| Group 1 Sham/Veh | | | | | | | | |
| Group 2 Bleo/Veh vs. | −18.85 | −19.98 | 1.135 | 5.685 | 13 | 11 | 0.1997 | 58 |
| Group 3 Bleo/BETi1 | | | | | | | | |
| 0.06 mg/ml | | | | | | | | |
| Group 2 Bleo/Veh vs. | −18.85 | −17.12 | −1.732 | 5.344 | 13 | 14 | 0.3242 | 58 |
| Group 4 Bleo/BETi1 | | | | | | | | |
| 0.6 mg/mL | | | | | | | | |
| Group 2 Bleo/Veh vs. | −18.85 | −20.64 | 1.788 | 5.258 | 13 | 15 | 0.3400 | 58 |
| Group 5 Bleo/BETi1 | | | | | | | | |
| 3 mg/mL | | | | | | | | |

²Each group is compared to group B (group 2 vehicle).

TABLE 43F
% Change Body Weight One Way ANOVA Week 3 multiple comparisons

| Dunnett's multiple | Mean | 95.00% | Below | | Adjusted | | |
|---|---|---|---|---|---|---|---|
| comparisons test | Diff. | CI of diff. | threshold? | Summary | P Value | B³ | |
| Group 2 Bleo/Veh vs. | −21.93 | −36.36 | Yes | ** | 0.0013 | A | Group 1 |
| Group 1 Sham/Veh | | to −7.492 | | | | | Sham/Veh |
| Group 2 Bleo/Veh vs. | −4.313 | −19.66 | No | ns | 0.8884 | C | Group 3 |
| Group 3 Bleo/BETi1 | | to 11.04 | | | | | Bleo/BETi1 |
| 0.06 mg/mL | | | | | | | 0.06 mg/mL |
| Group 2 Bleo/Veh vs. | 0.4395 | −13.10 | No | ns | 0.9999 | D | Group 4 |
| Group 4 Bleo/BETi1 | | to 13.97 | | | | | Bleo/BETi1 |
| 0.6 mg/mL | | | | | | | 0.6 mg/mL |
| Group 2 Bleo/Veh vs. | 3.600 | −9.935 | No | ns | 0.9057 | E | Group 5 |
| Group 5 Bleo/BETi1 | | to 17.13 | | | | | Bleo/BETi1 |
| 3 mg/mL | | | | | | | 3 mg/mL |

| | | | Mean | SE of | | | | |
|---|---|---|---|---|---|---|---|---|
| Test details | Mean 1 | Mean 2 | Diff. | diff. | n1 | n2 | q | DF |
| Group 2 Bleo/Veh vs. | −13.14 | 8.784 | −21.93 | 5.720 | 11 | 10 | 3.833 | 50 |
| Group 1 Sham/Veh | | | | | | | | |
| Group 2 Bleo/Veh vs. | −13.14 | −8.830 | −4.313 | 6.083 | 11 | 8 | 0.7090 | 50 |
| Group 3 Bleo/BETi1 | | | | | | | | |
| 0.06 mg/mL | | | | | | | | |
| Group 2 Bleo/Veh vs. | −13.14 | −13.58 | 0.4395 | 5.363 | 11 | 13 | 0.08194 | 50 |
| Group 4 Bleo/BETi1 | | | | | | | | |
| 0.6 mg/mL | | | | | | | | |
| Group 2 Bleo/Veh vs. | −13.14 | −16.74 | 3.600 | 5.363 | 11 | 13 | 0.6712 | 50 |
| Group 5 Bleo/BETi1 | | | | | | | | |
| 3 mg/mL | | | | | | | | |

³Each group is compared to group B (group 2 vehicle).

TABLE 43G
% Change Body Weight Two Way ANOVA multiple comparisons

| Dunnett's multiple | Predicted (LS) | 95.00% | Below | | Adjusted |
|---|---|---|---|---|---|
| comparisons test | mean diff. | CI of diff. | threshold? | Summary | P Value |
| Baseline | | | | | |
| Group 2 Bleo/Veh vs. Group 1 | 0.09875 | −2.092 to | No | ns | 0.9999 |
| Sham/Veh | | 2.290 | | | |
| Group 2 Bleo/Veh vs. Group 3 | 0.08750 | −1.834 to | No | ns | 0.9998 |
| Bleo/BETi1 0.06 mg/mL | | 2.009 | | | |
| Group 2 Bleo/Veh vs. Group 4 | 0.1750 | −1.746 to | No | ns | 0.9983 |
| Bleo/BETi1 0.6 mg/mL | | 2.096 | | | |
| Group 2 Bleo/Veh vs. Group 5 | 0.1629 | −1.730 to | No | ns | 0.9986 |
| Bleo/BETi1 3 mg/mL | | 2.056 | | | |
| Week 1 | | | | | |
| Group 2 Bleo/Veh vs. Group 1 | −0.2012 | −2.392 to | No | ns | 0.9982 |
| Sham/Veh | | 1.990 | | | |
| Group 2 Bleo/Veh vs. Group 3 | −0.2812 | −2.203 to | No | ns | 0.9898 |
| Bleo/BETi1 0.06 mg/mL | | 1.640 | | | |
| Group 2 Bleo/Veh vs. Group 4 | 0.1375 | −1.784 to | No | ns | 0.9991 |
| Bleo/BETi1 0.6 mg/mL | | 2.059 | | | |
| Group 2 Bleo/Veh vs. Group 5 | −0.2048 | −2.098 to | No | ns | 0.9968 |
| Bleo/BETi1 3 mg/mL | | 1.688 | | | |
| Week 2 | | | | | |
| Group 2 Bleo/Veh vs. Group 1 | −5.314 | −7.505 to | Yes | **** | <0.0001 |
| Sham/Veh | | −3.123 | | | |
| Group 2 Bleo/Veh vs. Group 3 | 0.3375 | −1.584 to | No | ns | 0.9800 |
| Bleo/BETi1 0.06 mg/mL | | 2.259 | | | |
| Group 2 Bleo/Veh vs. Group 4 | −0.3438 | −2.265 to | No | ns | 0.9786 |
| Bleo/BETi1 0.6 mg/mL | | 1.578 | | | |
| Group 2 Bleo/Veh vs. Group 5 | −0.05257 | −1.946 to | No | ns | >0.9999 |
| Bleo/BETi1 3 mg/mL | | 1.840 | | | |
| Week 3 | | | | | |
| Group 2 Bleo/Veh vs. Group 1 | −4.789 | −7.159 to | Yes | **** | <0.0001 |
| Sham/Veh | | −2.419 | | | |
| Group 2 Bleo/Veh vs. Group 3 | −2.409 | −4.930 to | No | ns | 0.0657 |
| Bleo/BETi1 0.06 mg/mL | | 0.1116 | | | |
| Group 2 Bleo/Veh vs. Group 4 | −0.1629 | −2.385 to | No | ns | 0.9991 |
| Bleo/BETi1 0.6 mg/mL | | 2.059 | | | |
| Group 2 Bleo/Veh vs. Group 5 | 0.6601 | −1.562 to | No | ns | 0.8787 |
| Bleo/BETi1 3 mg/mL | | 2.882 | | | |

TABLE 44A
Weight of Whole lung (grams)

| | Group 1 | Group 2 | Group 3 | Group 4 | Group 5 |
|---|---|---|---|---|---|
| Animal | Sham/ | Bleo/ | Bleo/BETi1 | Bleo/BETi1 | Bleo/BETi1 |
| # | Veh | Veh | 0.06 mg/mL | 0.6 mg/mL | 3 mg/mL |
| 1 | 0.16 | 0.28 | 0.30 | 0.20 | 0.21 |
| 2 | 0.14 | 0.29 | 0.19 | | 0.22 |
| 3 | 0.15 | 0.32 | 0.27 | 0.21 | 0.33 |
| 4 | 0.16 | 0.37 | 0.32 | 0.13 | 0.31 |
| 5 | 0.17 | 0.36 | 0.28 | 0.31 | 0.33 |
| 6 | 0.16 | 0.35 | | 0.29 | 0.30 |
| 7 | 0.17 | 0.23 | 0.25 | | 0.23 |
| 8 | 0.16 | 0.21 | 0.27 | 0.32 | 0.30 |
| 9 | 0.15 | | 0.27 | 0.37 | 0.27 |
| 10 | 0.13 | 0.32 | | 0.27 | 0.29 |
| 11 | | 0.20 | | | 0.34 |
| 12 | | 0.22 | | 0.20 | 0.31 |
| 13 | | | | 0.33 | |
| 14 | | | | 0.34 | |
| 15 | | | | 0.34 | 0.28 |
| 16 | | | | 0.18 | |
| Mean | 0.16 | 0.29 | 0.27 | 0.27 | 0.28 |
| s.d. | 0.01 | 0.06 | 0.04 | 0.07 | 0.04 |

TABLE 44B
Weight of Whole lung One Way ANOVA multiple comparisons

| Dunnett's multiple | Mean | 95.00% | Below | | Adjusted | | |
|---|---|---|---|---|---|---|---|
| comparisons test | Diff. | CI of diff. | threshold | Summary | P Value | B⁴ | |
| Group 2 Bleo/Veh vs. | 0.1296 | 0.07031 | Yes | **** | <0.0001 | A | Group 1 |
| Group 1 Sham/Veh | | to 0.1889 | | | | | Sham/Veh |
| Group 2 Bleo/Veh vs. | 0.01712 | −0.04591 | No | ns | 0.8993 | C | Group 3 |
| Group 3 Bleo/BETi1 | | to 0.08016 | | | | | Bleo/BETi1 |
| 0.06 mg/mL | | | | | | | 0.06 mg/mL |
| Group 2 Bleo/Veh vs. | 0.01849 | −0.03708 | No | ns | 0.8174 | D | Group 4 |
| Group 4 Bleo/BETi1 | | to 0.07407 | | | | | Bleo/BETi1 |
| 0.6 mg/mL | | | | | | | 0.6 mg/mL |
| Group 2 Bleo/Veh vs. | 0.001901 | −0.05367 | No | ns | 0.9999 | E | Group 5 |
| Group 5 Bleo/BETi1 | | to 0.05748 | | | | | Bleo/BETi1 |
| 3 mg/mL | | | | | | | 3 mg/mL |

| | | | Mean | SE of | | | | |
|---|---|---|---|---|---|---|---|---|
| Test details | Mean 1 | Mean 2 | Diff. | diff. | n1 | n2 | q | DF |
| Group 2 Bleo/Veh vs. | 0.2866 | 0.1570 | 0.1296 | 0.02349 | 11 | 10 | 5.517 | 50 |
| Group 1 Sham/Veh | | | | | | | | |
| Group 2 Bleo/Veh vs. | 0.2866 | 0.2695 | 0.01712 | 0.02498 | 11 | 8 | 0.6855 | 50 |
| Group 3 Bleo/BETi1 | | | | | | | | |
| 0.06 mg/mL | | | | | | | | |
| Group 2 Bleo/Veh vs. | 0.2866 | 0.2681 | 0.01849 | 0.02202 | 11 | 13 | 0.8398 | 50 |
| Group 4 Bleo/BETi1 | | | | | | | | |
| 0.6 mg/mL | | | | | | | | |
| Group 2 Bleo/Veh vs. | 0.2866 | 0.2847 | 0.001901 | 0.02202 | 11 | 13 | 0.08634 | 50 |
| Group 5 Bleo/BETi1 | | | | | | | | |
| 3 mg/mL | | | | | | | | |

⁴Each group is compared to group B (group 2 vehicle).

TABLE 45
Probability of Survival (%)

| | | Group2 | | | |
|---|---|---|---|---|---|
| | Group1 | Bleo- | Group 3 | Group 4 | Group 5 |
| Day | Sham/ | mycin/ | Bleo/BETi1 | Bleo/BETi1 | Bleo/BETi1 |
| Elapsed | Vehicle | Vehicle | 0.06 mg/mL | 0.6 mg/mL | 3 mg/mL |
| 0 | 100 | 100 | 100 | 100 | 100 |
| 1 | | 93.75 | | | |
| 8 | | | 93.75 | | |
| 9 | | | 81.25 | | |
| 10 | | 87.5 | 75 | 87.5 | |
| 11 | | 81.25 | | | |
| 12 | | 75 | 68.75 | | 88.24 |
| 14 | | | 62.5 | | |
| 15 | | 68.75 | | | |
| 16 | | 62.5 | | | |
| 18 | | | 56.25 | | |
| 19 | | | 50 | 81.3% | 76.5% |
| 20 | 100% | 62.5% | 50% | 81.3% | 76.5% |

TABLE 46A
Hydroxyproline Level of Lung (μg/mg tissue)

| | Group 1 | Group 2 | Group 3 | Group 4 | Group 5 |
|---|---|---|---|---|---|
| Animal | Sham/ | Bleo/ | Bleo/BETi1 | Bleo/BETi1 | Bleo/BETi1 |
| # | Veh | Veh | 0.06 mg/mL | 0.6 mg/mL | 3 mg/mL |
| 1 | 0.2256 | 0.1880 | 0.1322 | 0.3121 | 0.3024 |
| 2 | 0.1001 | 0.2996 | 0.1196 | 0.2982 | 0.1349 |
| 3 | 0.1308 | 0.1224 | 0.1977 | 0.3414 | 0.1029 |
| 4 | 0.1642 | 0.2633 | 0.1154 | 0.1377 | 0.1935 |
| 5 | 0.2256 | 0.3024 | 0.2842 | 0.3400 | 0.0596 |
| 6 | 0.2493 | 0.4056 | 0.3149 | 0.2954 | 0.2200 |
| 7 | 0.2605 | 0.4795 | 0.2968 | 0.1503 | 0.2800 |
| 8 | 0.2242 | 0.4921 | 0.3372 | 0.2745 | 0.2731 |
| 9 | 0.2884 | 0.3358 | | 0.3191 | 0.1308 |
| 10 | 0.2814 | 0.2940 | | 0.2242 | 0.2466 |
| 11 | | 0.2717 | | 0.2703 | 0.1112 |
| 12 | | | | 0.1029 | 0.3107 |
| 13 | | | | 0.2298 | 0.2382 |
| Mean | 0.2150 | 0.3140 | 0.2248 | 0.2535 | 0.2003 |
| s.d. | 0.0602 | 0.1069 | 0.0879 | 0.0763 | 0.0807 |

TABLE 46B
Hydroxyproline Level of Lung One Way ANOVA multiple comparisons

| Dunnett's multiple | Mean | 95.00% | Below | | Adjusted | | |
|---|---|---|---|---|---|---|---|
| comparisons test | Diff. | CI of diff. | threshold? | Summary | P Value | B⁵ | |
| Group 2 Bleo/Veh vs. | 0.09900 | 0.002379 | Yes | * | 0.0431 | A | Group 1 |
| Group 1 Sham/Veh | | to 0.1956 | | | | | Sham/Veh |
| Group 2 Bleo/Veh vs. | 0.08927 | −0.01348 | No | ns | 0.1055 | C | Group 3 |
| Group 3 Bleo/BETi1 | | to 0.1920 | | | | | Bleo/BETi1 |
| 0.06 mg/mL | | | | | | | 0.06 mg/mL |
| Group 2 Bleo/Veh vs. | 0.06050 | −0.03010 | No | ns | 0.2808 | D | Group 4 |
| Group 4 Bleo/BETi1 | | to 0.1511 | | | | | Bleo/BETi1 |
| 0.6 mg/mL | | | | | | | 0.6 mg/mL |
| Group 2 Bleo/Veh vs. | 0.1137 | 0.02313 | Yes | ** | 0.0095 | E | Group 5 |
| Group 5 Bleo/BETi1 | | to 0.2043 | | | | | Bleo/BETi1 |
| 3 mg/mL | | | | | | | 3 mg/mL |

| | | | Mean | SE of | | | | |
|---|---|---|---|---|---|---|---|---|
| Test details | Mean 1 | Mean 2 | Diff. | diff. | n1 | n2 | Q | DF |
| Group 2 Bleo/Veh vs. | 0.3140 | 0.2150 | 0.09900 | 0.03829 | 11 | 10 | 2.586 | 50 |
| Group 1 Sham/Veh | | | | | | | | |
| Group 2 Bleo/Veh vs. | 0.3140 | 0.2248 | 0.08927 | 0.04072 | 11 | 8 | 2.192 | 50 |
| Group 3 Bleo/BETi1 | | | | | | | | |
| 0.06 mg/mL | | | | | | | | |
| Group 2 Bleo/Veh vs. | 0.3140 | 0.2535 | 0.06050 | 0.03590 | 11 | 13 | 1.685 | 50 |
| Group 4 Bleo/BETi1 | | | | | | | | |
| 0.6 mg/mL | | | | | | | | |
| Group 2 Bleo/Veh vs. | 0.3140 | 0.2003 | 0.1137 | 0.03590 | 11 | 13 | 3.168 | 50 |
| Group 5 Bleo/BETi1 | | | | | | | | |
| 3 mg/mL | | | | | | | | |

⁵Each group is compared to group B (group 2 vehicle).

TABLE 47
Histopathology Scoring According
to the Hubner-modified Ashcroft

| | Group 1 | Group 2 | Group 3 | Group 4 | Group 5 |
|---|---|---|---|---|---|
| | Sham/ | Bleo/ | Bleo/BETi1 | Bleo/BETi1 | Bleo/BETi1 |
| | vehicle | vehicle | 0.06 mg/mL | 0.6 mg/mL | 3 mg/mL |
| Mean | 1.52 | 6.36 | 3.00 | 4.64 | 5.52 |
| Histology | | | | | |
| Score | | | | | |
| S.D | 0.66 | 0.88 | 0.72 | 0.82 | 0.78 |

### Section E:

### Toxicity and Toxicokinetics Study of Beti1 in Dogs

The purpose of this study was to determine the maximum tolerated dose and potential toxicity of BETi1 when administered by intra-articular injection to Beagle dogs and to determine local and systemic toxicokinetic characteristics associated with treatment of Beagle dogs with BETi1 formulations.

Experimental Method 5: Treatment of Beagle Dogs with BETi1 Formulations

16 Beagle dogs (8 males, 8 females) were assigned to four treatment groups to determine toxicity of BETi1 formulations following intra-articular injection. The dogs were observed for twenty eight days following injection.

The experimental groups are presented below:
Group 1: Control; 0 mg of BETi1/animal dose
Group 2: Low Dose; 7.5 mg of BETi1/animal dose
Group 3: Mid Dose; 75 mg of BETi1/animal dose
Group 4: High Dose; 150 mg of BETi1/animal dose

The control and BETi1 dose formulations were comprised as follows: Control: Vehicle (0.5% sodium carboxymethylcellulose; 0.1% polysorbate 80 in saline)
Low Dose: 0.75% w/w BETi1 suspended in Vehicle
Mid Dose: 7.5% w/w BETi1 suspended in Vehicle
High Dose: 15% w/w BETi1 suspended in Vehicle

Study dogs were administered control treatment or treatment with a BETi1 formulation via intra-articular injection only to their right knee joints. Dosage volume was 1 mL/knee.

### Sample Collection and Preparation

Blood samples were collected from all study dogs at 0 (pre-dose), 1, 2, 4, 8, and 24 hours post-dose. Blood samples were additionally collection from all study dogs at 168 h, 336 h, 504 h, and 672 h post-dose. Approximately 0.5 mL of blood was collected from animals via a cephalic or saphenous vein. Blood was collected into appropriately labeled tubes containing K₂EDTA as the anticoagulant. Plasma was obtained within two hours of collection by centrifugation at 3200×g and 4° C. for ten minutes. Plasma was then transferred into labeled clear polypropylene tubes and frozen in the upright position over dry ice and stored in a freezer set to maintain <−60° C. until shipped on dry ice for analysis.

Synovial fluid samples were collected from all study dogs at 72 h, 168 h, 336 h, 504 h, and 672 h post-dose. Approximately 80 μL of synovial fluid was collected from animals from the right knee joint and placed into appropriately labeled tubes onto wet ice. Synovia was then transferred into labeled clear polypropylene tubes and frozen in the upright position over dry ice and stored in a freezer set to maintain <−60° C. until shipped on dry ice for analysis.

### Sample Analysis

Quantification of BETi1 concentrations in plasma and synovial fluid was performed using a qualified liquid chromatographic triple quadrupole mass spectrometric (LC-MS/MS) method. Synovial fluids were centrifuged before analysis.

### Toxicokinetic Data Analysis

Toxicokinetic (TK) analysis of BETi1 plasma concentration-time and synovial fluid concentration-time data was performed. TK parameter values including maximum plasma/synovial fluid concentrations (Cₘₐₓ), time to reach maximum concentrations (Tₘₐₓ), and area under the plasma/synovial fluid concentrations vs. time curve (AUC) from time zero to 672-hour (AUC₀₋₆₇₂ₕ) was determined. Values that were below the lower limit of quantification (BLQ) were set to zero in the calculation of mean concentrations. When more than half of individual values at a single time point were BLQ, mean values were reported as BLQ.

Several features were measured in treated dogs, including viability, clinical observation, body weight, food consumption, clinical pathology (hematology, coagulation, serum chemistry, urinalysis), toxicokinetics, and pathology.

### Histopathology

For all animals from the study, representative samples of organs and all gross lesions and masses from all groups were removed from fixative, trimmed, processed, embedded in paraffin, sectioned, stained with hematoxylin and eosin, and examined microscopically. Two slides for each right kneejoint were prepared, one for E staining and another for Safranin 0 staining, and microscopic examination was conducted for the right knee joint. A modified Semi-Quantitative Mankin scoring system was used to in evaluation of right knee joint histopathology, as shown in Table 48 below:

TABLE 48
Semi-Quantitative Mankin scoring

| Category | Score |
|---|---|
| Structure | |
| Normal | 0 |
| Surface irregularities | 1 |
| Pannus and surface irregularities | 2 |
| Clefts to transitional zone | 3 |
| Clefts to tidemark | 4 |
| Clefts to subchondral bone | 5 |
| Complete disorganization | 6 |
| Cells | |
| Normal | 0 |
| Diffuse hypercellularity | 1 |
| Cloning | 2 |
| Hypocellularity (necrosis) | 3 |
| Safranin O staining | |
| Normal | 0 |
| Slight reduction, tangential zone | 1 |
| Moderate reduction (to tidemark) | 2 |
| Severe reduction (to subchondral bone in at least 1 area) | 3 |
| No dye observed | 4 |
| Tidemark Integrity | |
| Intact | 0 |
| Crossed by blood vessels | 1 |
| Total | 0-14 |
| Intact | 0 |

### Example 11—Toxicokinetics Following Intra-Articular Injection

### Results

TABLE 48A
Synovial concentrations of BETi1 following intra-articular injection
of Beagle dogs with formulations comprising 7.5 mg, 75 mg, or 150
mg of BETi1 at 72 h, 168 h, 336 h, 504 h, and 672 post-treatment

| | | Days | | | | |
|---|---|---|---|---|---|---|
| | | 3 | 7 | 14 | 21 | 28 |
| | | Hours | | | | |
| | | 72 | 168 | 336 | 504 | 672 |
| 7.5 | mg | 125.625 | 1.58 | 0 | 0 | 0.685 |
| 75 | mg | 27050 | 33700 | 6805 | 937 | 316 |
| 150 | mg | 47600 | 36000 | 18070 | 7765 | 4375 |

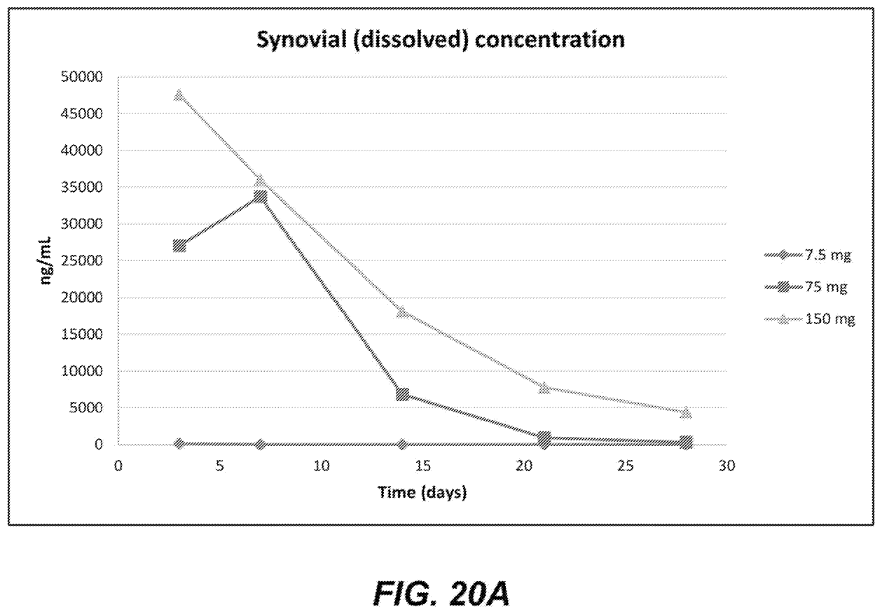

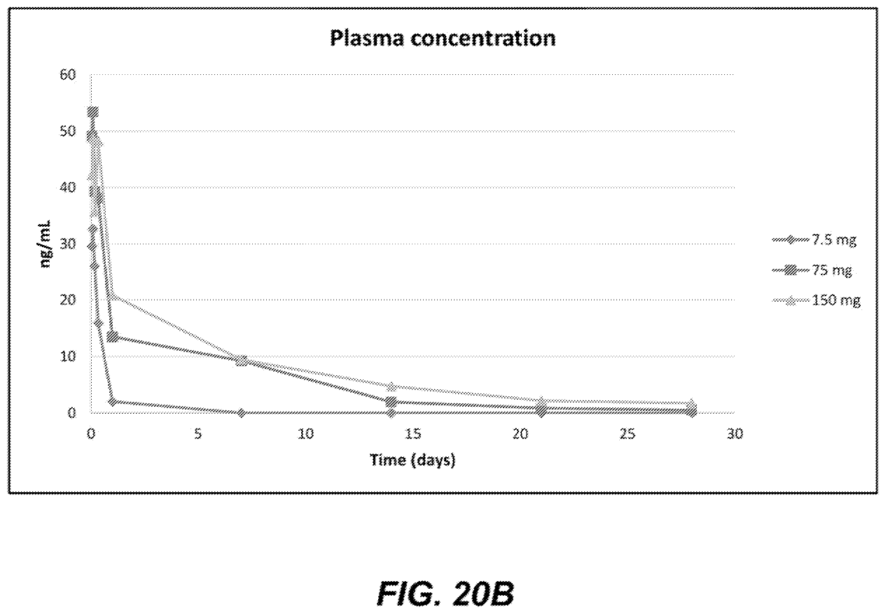

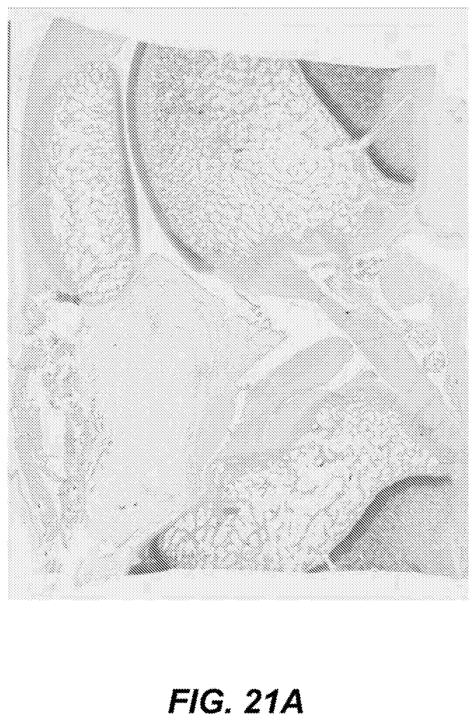

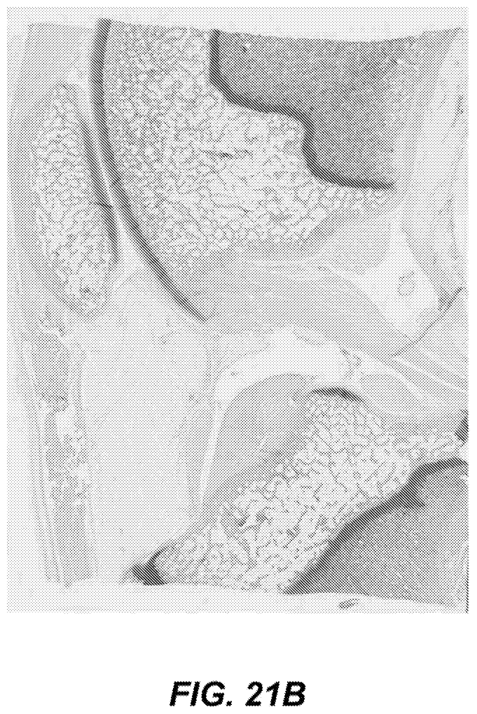

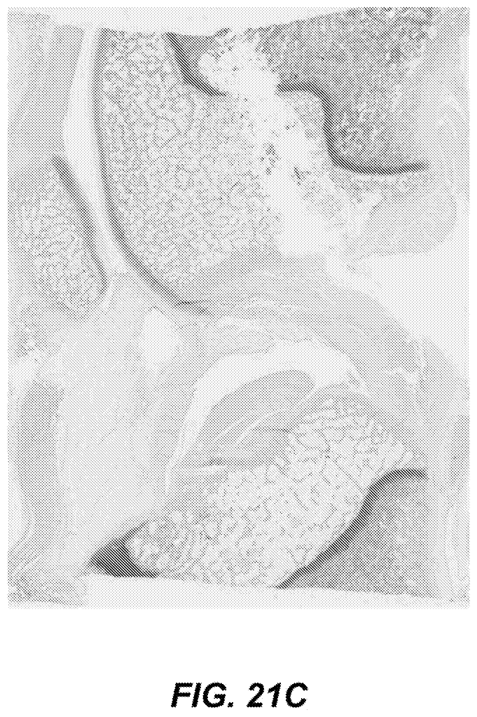

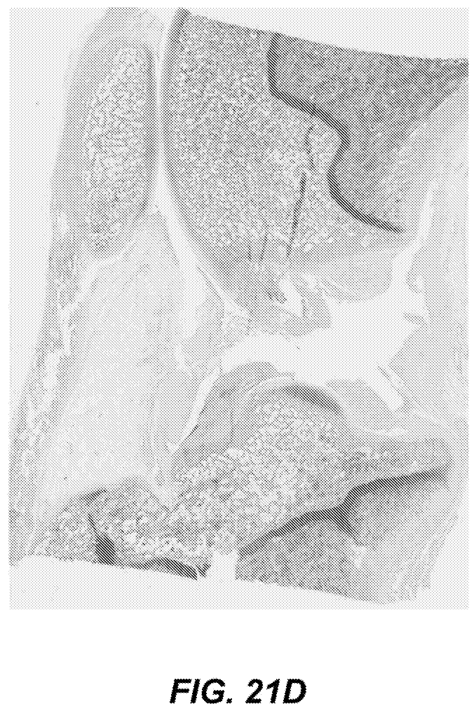

Synovial concentrations of BETi1 correlating to the findings in Table 48A are depicted graphically in FIG. 20A. Calculated Tₘₐₓ was about 5 days and Cₘₐₓ reached the BETi1 water solubility limit (50 μg/mL) for the highest dose. For the two highest doses, meaningful synovial levels were still detectable at four weeks post-treatment (1 μM and 13 μM, respectively, for the 75 mg and 150 mg doses).

TABLE 48B
Plasma concentrations of BETi1 following intra-articular injection of Beagle
dogs with formulations comprising 7.5 mg, 75 mg, or 150 mg of BETi1 at
1 h, 2 h, 4 h, 8 h, 24 h, 168 h, 336 h, 504 h, and 672 post-treatment

| | | Days | | | | | | | | |
|---|---|---|---|---|---|---|---|---|---|---|
| | | 0.04 | 0.08 | 0.16 | 0.33 | 1 | 7 | 14 | 21 | 28 |
| | | Hours | | | | | | | | |
| | | 1 | 2 | 4 | 8 | 24 | 168 | 336 | 504 | 672 |
| 7.5 | mg | 29.6 | 32.6 | 26.05 | 15.95 | 2.025 | 0 | 0 | 0 | 0 |
| 75 | mg | 49.1 | 53.4 | 39.35 | 38.15 | 13.5 | 9.22 | 1.95 | 0.835 | 0.505 |
| 150 | mg | 42.25 | 48.75 | 35.75 | 48.25 | 20.85 | 9.43 | 4.72 | 2.195 | 1.72 |

Plasma concentrations of BETi1 correlating to the findings in Table 48B are depicted graphically in FIG. 20B. Compared to synovial BETi1 levels, plasma Cₘₐₓ was ˜1000× lower, AUC was ˜2000× lower, and BETi1 levels were up to about 5000× lower. Calculated Tₘₐₓ was about 5 days and Cₘₐₓ reached only 50 ng/mL. For the two highest doses, low plasma levels were still detectable at four weeks post-treatment (2 nM and 5 nM, respectively, for the 75 mg and 150 mg doses).

TABLE 49A
Plasma Cₘₐₓ, Tₘₐₓ, and AUC values in male and female Beagle
dogs following treatment with 7.5 mg, 75 mg, or 150 mg BETi1
Plasma

| Dose | Study | | Cₘₐₓ | Tₘₐₓ | AUC_{0-672 h} |
|---|---|---|---|---|---|
| (mg/animal) | Day | Sex | (ng/mL) | (h) | (h*ng/mL) |
| 7.5 | 1 | Male | 32.1 | 4.0 | 390 |
| | | Female | 37.6 | 1.5 | 472 |
| 75 | 1 | Male | 50.2 | 2.0 | 3070 |
| | | Female | 56.6 | 2.0 | 3760 |
| 150 | 1 | Male | 48.8 | 2.0 | 3810 |
| | | Female | 52.9 | 5.0 | 5990 |

TABLE 49B
Synovia Cₘₐₓ, Tₘₐₓ, and AUC values in male and female Beagle
dogs following treatment with 7.5 mg, 75 mg, or 150 mg BETi1
Synovia

| Dose | Study | | Cₘₐₓ | Tₘₐₓ | AUC_{0-672 h} |
|---|---|---|---|---|---|
| (mg/animal) | Day | Sex | (ng/mL) | (h) | (h*ng/mL) |
| 7.5* | 1 | Male | 244 | 120.0 | 25400 |
| 75 | 1 | Male | 47600 | 120.0 | 7890000 |
| | | Female | 43700 | 120.0 | 6090000 |
| 150 | 1 | Male | 63200 | 120.0 | 12500000 |
| | | Female | 46100 | 204.0 | 11300000 |

*Since most of the values at different timepoint were BLQ, the TK parameters were not calculated for female at 7.5 mg/animal.

### Histology

No test article-related mortality occurred. There were no test article-related changes in organ weights and gross examinations.

Test articles were overall well-tolerated, only some minimal or mild, focal/multifocal/locally extensive necrosis occurred in the synovium.

All the study animals including the concurrent control animals had a total Mankin score of zero except for one animal at 75 mg dose which only had a two points score in the 14-points scale.

### Summary

This dog study demonstrated that BETi1 is present in the joint in substantial amounts at the two higher tested concentrations (75 mg and 150 mg doses) and decreases over time. Of note, even at week 4, micromolar synovial levels are still present, and which may have a therapeutic effect. The data also showed that there is a very high synovial to plasma ratio, indicating that systemic exposure and effects should be very low even at the two highest doses tested. Further, the plasma levels indicate a rapid clearance with a reduction being seen after 4 hours. Overall, the study showed that the drug is well tolerated.

1. A method for the treatment, amelioration, or prophylaxis of an inflammatory and/or an autoimmune disease or disorder or a disease or disorder related thereto, the method comprising administering to a subject in need thereof a therapeutically effective amount of a compound of Formula (I) or a tautomer, stereoisomer or a mixture of stereoisomers, or a pharmaceutically acceptable salt, or hydrate, or deuterated derivative thereof: 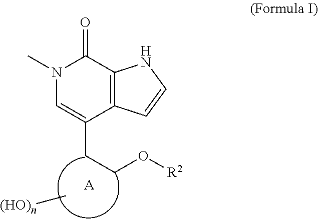
wherein:
ring A is selected from phenyl, N-methyl-2-pyridone, and thiazole;
n is 0 or 1, wherein when A is phenyl, n is 1; when A is N-methyl-2-pyridone, n is 0; and when A is thiazole, n is 0;
R² is phenyl optionally substituted with 1 to 3 substituents independently selected from hydroxy, halo, C₁-C₆ alkyl, C₃-C₆ cycloalkyl, C₁-C₅ alkyloxy, C₁-C₅ alkylamino, C₁-C₆ fluoroalkyl, C₁-C₅ fluoroalkyloxy, and C₁-C₅ fluoroalkylamino; C₂-C₆ alkyl; and C₃-C₆ cycloalkyl optionally substituted with a substituent selected from C₁-C₆ alkoxy.

2. The method of claim 1, wherein the disease or disorder is selected from:
a. wounds, wounds resistant to healing, wounds swelling, burns, psoriasis, Pyoderma gangrenosum (PG), Generalized Pustular Psoriasis (GPP), and Palmar Plantar Pustulosis (PPP);
b. a pigmentation or pigmentation related disease or disorder;
c. a joint or joint related disease or disorder;
d. a respiratory or respiratory related disease or disorder; and
e. a fibrosis or fibrosis-associated disease or disorder.

3. The method of claim 2, wherein the pigmentation or pigmentation related disease or disorder comprises vitiligo, chemical leukoderma, tinea versicolor spots, albinism, and pityriasis alba, atrophie blanche, Griscelli syndrome, Halo moles, Hermansky-Pudlak syndrome, Hypomelanosis of Ito, Idiopathic guttate hypomelanosis, Leprosy, Leukoderma, Lichen sclerosus, Lupus erythematosus, Morphoea, Mycosis fungoides, Naevus anaemicus, Naevus depigmentosus, Piebaldism, Pityriasis versicolor, Poliosis, Postinflammatory hypopigmentation, Progressive macular hypopigmentation, Tuberous sclerosis (ashleaf spots), and Waardenburg syndrome.

4. (canceled)

5. The method of claim 2, wherein the joint related disease or disorder comprises arthritis, bursitis, Ehlers-Danlos syndrome, epicondylitis, Felty Syndrome, gouty arthritis, psoriatic arthritis, osteoarthritis, rheumatoid arthritis, Still's disease, tenosynovitis, synovitis, Sjögren's Syndrome, lyme disease, Whipple disease, bone cancer, lupus, and other autoimmune joint disorders.

6-7. (canceled)

8. The method of claim 2, wherein the fibrosis or fibrosis-associated disorder and respiratory or respiratory related disorder comprises a lung disorder chosen from a pulmonary fibrosis (PF), idiopathic pulmonary fibrosis (IPF), desquamative interstitial pneumonia (DIP), acute interstitial pneumonia (AIP), nonspecific interstitial pneumonia (NSIP), respiratory bronchiolitis-associated interstitial lung disease (RB-ILD), cryptogenic organizing pneumonia (COP), and lymphoid interstitial pneumonia (LIP) or is a secondary lung disorder.

9-10. (canceled)

11. The method of claim 2, wherein the fibrosis or fibrosis related disorder comprises scarring and scar formation.

12. The method of claim 1, wherein
ring A is selected from

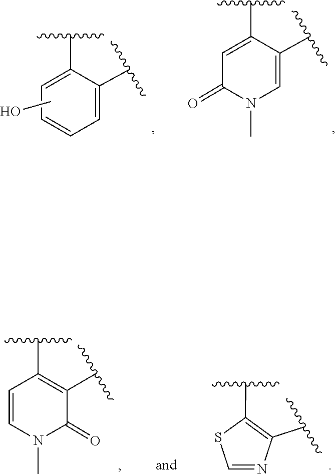

13-26. (canceled)

27. The method of claim 1, wherein the compound,
tautomer, stereoisomer, pharmaceutically acceptable salt, hydrate, and/or deuterated derivative thereof has activity against one or more BET domains and/or
wherein there is an improvement in at least one symptom of the disorder or disease upon topical application of a therapeutically effective amount of compound, tautomer, stereoisomer, pharmaceutically acceptable salt, hydrate, and/or deuterated derivative thereof.

28. (canceled)

29. The method of claim 1, wherein upon administration of a therapeutically effective amount of the compound, tautomer, stereoisomer, pharmaceutically acceptable salt, hydrate, and/or deuterated derivative thereof there is a reduction in one or more cytokines that are elevated in inflammation.

30. The method of claim 29, wherein the one or more cytokines are chosen from SOX9, POMC, IL-6, IL-1α, IL-1β, TNF-α, and EDN1, or chosen from IL-1β, IL-6, IL-8, IL-17, IL-18, IL-23, IL-36 and TNF-α, or chosen from IL-1β, IL-17, IL-6, IL-36 and TNF-α, or chosen from IL-1β, IL-17, TNF-α, IL-8, IL-6 and IL-23.

31. The method of claim 3, to wherein upon administration of a therapeutically effective amount of the compound, tautomer, stereoisomer, pharmaceutically acceptable salt, hydrate, and/or deuterated derivative thereof a therapeutic effect is associated with one or more of the following: a) an upregulation of the WNT pathway or signaling including WNT and/or upregulation of RABA3A, b) a reduction in MMP9 and/or soluble E-cadherin, and c) a reduction in melanocytorhagy.

32-34. (canceled)

35. The method of claim 1, wherein
upon administration of a therapeutically effective amount of the compound, tautomer, stereoisomer, pharmaceutically acceptable salt, hydrate, and/or deuterated derivative thereof a therapeutic effect is associated with a reduction at the site of the disorder in infiltrates of lymphocytes, macrophages, and neutrophils.

36-38. (canceled)

39. The method of claim 1, wherein the disorder is an arthritis and upon administration of a therapeutically effective amount of the compound, tautomer, stereoisomer, pharmaceutically acceptable salt, hydrate, and/or deuterated derivative thereof a therapeutic effect is associated with dose-dependent reduction in inflammation is a reduction in thickness or girth of a joint or limb.

40. The method of claim 39, wherein there is reduction in arthritic scoring or severity, and
wherein the reduction in arthritic scoring or severity is a reduction in:
(a) definite redness and swelling of the ankle/wrist or apparent redness and swelling limited to individual digits, regardless of the number of affected digits;
(b) severe redness and swelling of the ankle/wrist;
(c) redness and swelling of the entire paw including digits; and/or
(d) maximally inflamed limb with involvement of multiple joints.

41. The method of claim 29, wherein the reduction is dose dependent.

42. The method of claim 29, wherein the reduction is by greater than about 50%.

43. The method of claim 31, wherein the upregulation is by greater than about 50%.

44. The method of claim 1, wherein the compound or a tautomer, stereoisomer or a mixture of stereoisomers thereof, or a pharmaceutically acceptable salt, or hydrate, or deuterated derivative thereof is in the form of a pharmaceutical composition which comprises a compound of Formula (I), or a tautomer, stereoisomer or a mixture of stereoisomers, or a pharmaceutically acceptable salt or hydrate, or deuterated derivative thereof, and a pharmaceutically acceptable carrier.

45. The method of claim 44, wherein the compound, tautomer, stereoisomer, pharmaceutically acceptable salt, hydrate, and/or deuterated derivative thereof is formulated as a suspension or partial suspension in the composition.

46. The method of claim 45, wherein the compound, tautomer, stereoisomer, pharmaceutically acceptable salt, hydrate, and/or deuterated derivative thereof is micronized and/or comprises nanoparticles.

47. (canceled)

48. The method of claim 44, wherein the compound, tautomer, stereoisomer, pharmaceutically acceptable salt, hydrate, and/or deuterated derivative thereof is solubilized or partially solubilized in the composition.

49. The method according to claim 44, wherein the compound, tautomer, stereoisomer, pharmaceutically acceptable salt, hydrate, and/or deuterated derivative thereof or pharmaceutical composition is administered by one or more of the following routes of administration: (a) locally, topically or systemically, (b) single injection, sequential injections, or infusion, and (c) inhalation, intrapulmonary, or nasally.

50. (canceled)

51. The method according to claim 49, wherein the administration is into the epidural space, other spinal space, foramenal space, intraarticular space, lesions, periarticular space, perineum space, soft tissues, or at or near the location of inflammation, pain and/or damage.

52. (canceled)

53. The method according to claim 1, wherein the composition comprises, one or more ingredients chosen from PLGA microspheres, a carboxymethyl cellulose, a hyaluronic acid and a lubricant or a lubricin.

54. The method of claim 49, wherein when injected intraarticularly in a therapeutically effective amount the concentration of dissolved compound within the synovial fluid at 14 days is greater than about 3000 fold than that in the plasma and/or between days 7 to 21 ranges from greater than or equal to about 5000 fold to equal to or less than about 1000 fold than that in the plasma.

55. The method of claim 1, wherein the disease or disorder is a wound and, upon administration of a therapeutically effective amount of the compound, tautomer, stereoisomer, pharmaceutically acceptable salt, hydrate, and/or deuterated derivative thereof, a therapeutic effect is associated with one or more of the following: a) improved scar outcome, b) improved lesion outcome, c) reduced fibrotic tissue in lesion, d) less tissue mass under a scar, e) reduced visibility of scar, f) less distinct scar, and g) an improved aesthetic outcome.
